(19) United States
(12) Patent Application Publication
(10) Pub. No.: US 2025/0043281 A1
(43) Pub. Date: Feb. 6, 2025

(54) TREATMENT OF FGG RELATED DISEASES AND DISORDERS

(71) Applicant: Empirico Inc., San Diego, CA (US)

(72) Inventors: Omri GOTTESMAN, San Diego, CA (US); Shannon BRUSE, San Diego, CA (US); Brian CAJES, San Diego, CA (US); David LEWIS, Madison, WI (US); David ROZEMA, Cross Plains, WI (US); John VEKICH, San Diego, CA (US)

(21) Appl. No.: 18/714,537

(22) PCT Filed: Dec. 5, 2022
(86) PCT No.: PCT/US22/80933

§ 371 Date: May 29, 2024

Related U.S. Application Data
(60) Provisional application No. 63/286,393, filed on Dec. 6, 2021.

(51) Int. Cl. C12N 15/113; A61P 25/24; A61P 25/28

(52) U.S. Cl. CPC C12N 15/113; A61P 25/24; A61P 25/28; C12N 2310/11; C12N 2310/14; C12N 2310/315; C12N 2310/321; C12N 2310/322; C12N 2310/3231; C12N 2310/351

(57) ABSTRACT

Disclosed herein are compositions comprising an oligonucleotide that targets FGG. The oligonucleotide may include a small interfering RNA (siRNA) or an antisense oligonucleotide (ASO). Also provided herein are methods of treating a mental disorder, or a condition associated with an FGG mutation. The method may include providing an oligonucleotide to a subject that targets FGG.

### CROSS-REFERENCE

This application claims the benefit of U.S. Provisional No. 63/286,393, filed Dec. 6, 2021, which application is incorporated herein by reference.

### INCORPORATION BY REFERENCE OF SEQUENCE LISTING

The present application is being filed along with a Sequence Listing in electronic format. The Sequence Listing is provided as a file entitled 54462-736601.xml, created Dec. 3, 2022, which is 5155 kilobytes in size. The information in the electronic format of the Sequence Listing is incorporated by reference in its entirety.

### BACKGROUND

Psychiatric and neurological diseases are widely abundant, and may affect a wide variety of people. Improved therapeutics are needed for treating these disorders.

### SUMMARY

Disclosed herein, in some embodiments, are compositions comprising an oligonucleotide that targets fibrinogen gamma gene (FGG). Disclosed herein, in some embodiments, are compositions comprising an oligonucleotide that targets FGG and when administered to a subject in an effective amount improves a mental disorder measurement of a mental disorder. In some embodiments, the mental disorder comprises a psychiatric disorder. In some embodiments, the psychiatric disorder comprises a depressive disorder (e.g., major depressive disorder, persistent depressive disorder, treatment resistant depression and signs or symptoms of depression), post-traumatic stress disorder, mood disorder, anxiety disorder, eating disorder, substance-use disorder, bipolar disorder, personality disorder, schizophrenia, or schizoaffective disorder. In some embodiments, the mental disorder measurement is chosen from the group consisting of a Montgomery-Asberg Depression Rating Scale (MADRS) score, a Hamilton Depression Rating Scale-17 score, anxiety signs and symptoms, eating disorder signs and symptoms, substance-use disorder signs and symptoms, post-traumatic stress disorder signs and symptoms, bipolar disorder signs and symptoms, schizophrenia signs and symptoms, and psychosis signs and symptoms. In some embodiments, the mental disorder comprises a neurological disorder. In some embodiments, the neurological disorder comprises Alzheimer's disease, dementia, delirium, cognitive decline, vascular dementia, headache (e.g., migraine), chronic pain (e.g., fibromyalgia), chronic fatigue syndrome (e.g. myalgic encephalomyelitis), or motor neuron disease (e.g., amyotrophic lateral sclerosis). In some embodiments, the mental disorder measurement is chosen from the group consisting of cognitive function, CNS amyloid plaques, CNS tau accumulation, CSF beta-amyloid 42, CSF tau, CSF phospho-tau, Lewy bodies, CSF alpha-synuclein, headache symptoms or signs, migraine symptoms or signs, chronic pain symptoms or signs, fibromyalgia symptoms or signs, chronic fatigue syndrome (e.g. myalgic encephalomyelitis) symptoms or signs, and motor neuron disease (e.g., amyotrophic lateral sclerosis) symptoms or signs. In some embodiments, the oligonucleotide comprises a modified internucleoside linkage. In some embodiments, the modified internucleoside linkage comprises alkylphosphonate, phosphorothioate, methylphosphonate, phosphorodithioate, alkylphosphonothioate, phosphoramidate, carbamate, carbonate, phosphate triester, acetamidate, or carboxymethyl ester, or a combination thereof. In some embodiments, the modified internucleoside linkage comprises one or more phosphorothioate linkages. In some embodiments, the oligonucleotide comprises 1, 2, 3, 4, 5, 6, 7, 8, 9, 10, 11, 12, 13, 14, 15, 16, 17, 18, 19, or 20 modified internucleoside linkages. In some embodiments, the oligonucleotide comprises a modified nucleoside. In some embodiments, the modified nucleoside comprises a locked nucleic acid (LNA), hexitol nucleic acid (HLA), cyclohexene nucleic acid (CeNA), 2′-methoxyethyl, 2′-O-alkyl, 2′-O-allyl, 2′-O-allyl, 2′-fluoro, or 2′-deoxy, or a combination thereof. In some embodiments, the modified nucleoside comprises a LNA. In some embodiments, the modified nucleoside comprises a 2′,4′ constrained ethyl nucleic acid. In some embodiments, the modified nucleoside comprises a 2′-O-methyl nucleoside, 2′-deoxyfluoro nucleoside, 2′-O—N-methylacetamido (2′-O-NMA) nucleoside, a 2′-O-dimethylaminoethoxyethyl (2′-O-DMAEOE) nucleoside, 2′-O-aminopropyl (2′-O-AP) nucleoside, or 2′-ara-F, or a combination thereof. In some embodiments, the modified nucleoside comprises one or more 2′fluoro modified nucleosides. In some embodiments, the modified nucleoside comprises a 2′ O-alkyl modified nucleoside. In some embodiments, the oligonucleotide comprises 1, 2, 3, 4, 5, 6, 7, 8, 9, 10, 11, 12, 13, 14, 15, 16, 17, 18, 19, 20, or 21 modified nucleosides. In some embodiments, the oligonucleotide comprises a lipid attached at a 3′ or 5′ terminus of the oligonucleotide. In some embodiments, the lipid comprises cholesterol, myristoyl, palmitoyl, stearoyl, lithocholoyl, docosanoyl, docosahexaenoyl, myristyl, palmityl stearyl, or α-tocopherol, or a combination thereof. In some embodiments, the oligonucleotide comprises a sugar moiety attached at a 3′ or 5′ terminus of the oligonucleotide. In some embodiments, the sugar comprises N-acetylgalactosamine (GalNAc), N-acetylglucosamine (GlcNAc), or mannose. The sugar moiety may comprise ETL17. In some embodiments, the oligonucleotide comprises a small interfering RNA (siRNA) comprising a sense strand and an antisense strand. In some embodiments, the sense strand is 12-30 nucleosides in length. In some embodiments, the antisense strand is 12-30 nucleosides in length. Disclosed herein, in some embodiments, are compositions comprising an oligonucleotide that inhibits the expression of FGG, wherein the oligonucleotide comprises an siRNA comprising a sense strand and an antisense strand, each strand is independently about 12-30 nucleosides in length, and at least one of the sense strand and the antisense strand comprises a nucleoside sequence comprising about 12-30 contiguous nucleosides of SEQ ID NO: 3621. In some embodiments, any one of the following is true with regard to the sense strand: all purines comprise 2′ fluoro modified purines, and all pyrimidines comprise a mixture of 2′ fluoro and 2′-O-methyl modified pyrimidines; all purines comprise 2′-O-methyl modified purines, and all pyrimidines comprise a mixture of 2′ fluoro and 2′-O-methyl modified pyrimidines; all purines comprise 2′ fluoro modified purines, and all pyrimidines comprise 2′-O-methyl modified pyrimidines; all pyrimidines comprise 2′ fluoro modified pyrimidines, and all purines comprise a mixture of 2′ fluoro and 2′-O-methyl modified purines; all pyrimidines comprise 2′-O-methyl modified pyrimidines, and all purines comprise a mixture of 2′ fluoro and 2′-O-methyl modified purines; or all pyrimidines comprise 2′ fluoro modified pyrimidines, and all purines comprise 2′-O-methyl modified purines. In some embodiments, the sense strand comprises any one of modification patterns 1S, 2S, 3S, 4S, 5S, 6S, 7S, 8S, 9S, 10S, 11S, 12S, 13S, 14S, 15S, 156S, 17S, 18S, 19S, 20S, 21S, 22S, 23S, 24S, 25S, 28S, or 29S. In some embodiments, any one of the following is true with regard to the antisense strand: all purines comprise 2′ fluoro modified purines, and all pyrimidines comprise a mixture of 2′ fluoro and 2′-O-methyl modified pyrimidines; all purines comprise 2′-O-methyl modified purines, and all pyrimidines comprise a mixture of 2′ fluoro and 2′-O-methyl modified pyrimidines; all purines comprise 2′-O-methyl modified purines, and all pyrimidines comprise 2′ fluoro modified pyrimidines; all pyrimidines comprise 2′ fluoro modified pyrimidines, and all purines comprise a mixture of 2′ fluoro and 2′-O-methyl modified purines; all pyrimidines comprise 2′-O-methyl modified pyrimidines, and all purines comprise a mixture of 2′ fluoro and 2′-O-methyl modified purines; or all pyrimidines comprise 2′-O-methyl modified pyrimidines, and all purines comprise 2′ fluoro modified purines. In some embodiments, the antisense strand comprises any one of modification patterns 1AS, 2AS, 3AS, 4AS, 5AS, 6AS, 7AS, 8AS, 9AS, 10AS, 11AS, 12AS, 13AS, 14AS, 15AS, 16AS, 17AS, 18AS, 19AS, 20AS, or 21AS. In some embodiments, the sense strand comprises the nucleic acid sequence of any one of SEQ ID NOs: 1-1742 or 3713-3748, or a sequence thereof having 1 or 2 substitutions, additions, or deletions; and the antisense strand comprises the nucleic acid sequence of any one of SEQ ID NOs: 1743-3484 or 3749-3784, or a sequence thereof having 1 or 2 substitutions, additions, or deletions. In some embodiments, the sense strand comprises the nucleic acid sequence of any one of SEQ ID NOs: 1-1742 or 3713-3748, and the antisense strand comprises the nucleic acid sequence of any one of SEQ ID NOs: 1743-3484 or 3749-3784. In some embodiments, the sense strand comprises the nucleic acid sequence of any one of SEQ ID NOs: 3723, 3724, 3726, or 3747, or a sequence thereof having 1 or 2 substitutions, additions, or deletions; and the antisense strand comprises the nucleic acid sequence of any one of SEQ ID NOs: 3759, 3760, 3762, or 3783, or a sequence thereof having 1 or 2 substitutions, additions, or deletions. In some embodiments, the sense strand comprises the nucleic acid sequence of any one of SEQ ID NOs: 3723, 3724, 3726, or 3747, and the antisense strand comprises the nucleic acid sequence of any one of SEQ ID NOs: 3759, 3760, 3762, or 3783. In some embodiments, the sense strand comprises the nucleic acid sequence of any one of SEQ ID NOs: 352, 1003, 1011, or 1278, or a sequence thereof having 1 or 2 substitutions, additions, or deletions; and the antisense strand comprises the nucleic acid sequence of any one of SEQ ID NOs: 2094, 2745, 2753, or 3020, or a sequence thereof having 1 or 2 substitutions, additions, or deletions. In some embodiments, the sense strand comprises the nucleic acid sequence of any one of SEQ ID NOs: 352, 1003, 1011, or 1278, and the antisense strand comprises the nucleic acid sequence of any one of SEQ ID NOs: 2094, 2745, 2753, or 3020. In some embodiments, the sense strand comprises the nucleic acid sequence of any one of SEQ ID NOs: 3591-3594, or a sequence thereof having 1 or 2 substitutions, additions, or deletions; and the antisense strand comprises the nucleic acid sequence of any one of SEQ ID NOs: 3595-3598, or a sequence thereof having 1 or 2 substitutions, additions, or deletions. In some embodiments, the sense strand comprises the nucleic acid sequence of any one of SEQ ID NOs: 3591-3594, and the antisense strand comprises the nucleic acid sequence of any one of SEQ ID NOs: 3595-3598. In some embodiments, the oligonucleotide comprises an antisense oligonucleotide (ASO). In some embodiments, the ASO is 12-30 nucleosides in length. Disclosed herein, in some embodiments, are compositions comprising an oligonucleotide that inhibits the expression of FGG, wherein the oligonucleotide comprises an ASO about 12-30 nucleosides in length and a nucleoside sequence complementary to about 12-30 contiguous nucleosides of SEQ ID NO: 3621. Some embodiments include a pharmaceutically acceptable carrier. Disclosed herein, in some embodiments, are methods of treating a subject having a psychiatric disorder or a neurological disorder, comprising administering an effective amount of the composition to the subject. In some embodiments, the psychiatric disorder comprises a depressive disorder (e.g., major depressive disorder, persistent depressive disorder, treatment resistant depression and signs or symptoms of depression), post-traumatic stress disorder, mood disorder, anxiety disorders, eating disorder, substance-use disorder, bipolar disorder, personality disorder, schizophrenia, or a schizoaffective disorder. In some embodiments, the neurological disorder comprises Alzheimer's disease, dementia, delirium, cognitive decline, vascular dementia, headache (e.g., migraine), chronic pain (e.g., fibromyalgia), chronic fatigue syndrome (e.g. myalgic encephalomyelitis), and motor neuron disease (e.g., amyotrophic lateral sclerosis).

### BRIEF DESCRIPTION OF THE DRAWINGS

FIG. 1 is an example of a GalNAc ligand.

FIG. 2 is an example of a GalNAc ligand.

### DETAILED DESCRIPTION

Large-scale human genetic data can improve the success rate of pharmaceutical discovery and development. A Genome Wide Association Study (GWAS) may detect associations between genetic variants and traits in a population sample. A GWAS may enable better understanding of the biology of disease, and provide applicable treatments. A GWAS can utilize genotyping and/or sequencing data, and often involves an evaluation of millions of genetic variants that are relatively evenly distributed across the genome. The most common GWAS design is the case-control study, which involves comparing variant frequencies in cases versus controls. If a variant has a significantly different frequency in cases versus controls, that variant is said to be associated with disease. Association statistics that may be used in a GWAS are p-values, as a measure of statistical significance; odds ratios (OR), as a measure of effect size; or beta coefficients (beta), as a measure of effect size. Researchers often assume an additive genetic model and calculate an allelic odds ratio, which is the increased (or decreased) risk of disease conferred by each additional copy of an allele (compared to carrying no copies of that allele). An additional concept in design and interpretation of GWAS is that of linkage disequilibrium, which is the non-random association of alleles. The presence of linkage disequilibrium can obfuscate which variant is “causal.”

Functional annotation of variants and/or wet lab experimentation can identify the causal genetic variant identified via GWAS, and in many cases may lead to the identification of disease-causing genes. In particular, understanding the functional effect of a causal genetic variant (for example, loss of protein function, gain of protein function, increase in gene expression, or decrease in gene expression) may allow that variant to be used as a proxy for therapeutic modulation of the target gene, or to gain insight into potential therapeutic efficacy and safety of a therapeutic that modulates that target.

Identification of such gene-disease associations has provided insights into disease biology and may be used to identify novel therapeutic targets for the pharmaceutical industry. In order to translate the therapeutic insights derived from human genetics, disease biology in patients may be exogenously ‘programmed’ into replicating the observation from human genetics. There are several potential options for therapeutic modalities that may be brought to bear in translating therapeutic targets identified via human genetics into novel medicines. These may include well established therapeutic modalities such as small molecules and monoclonal antibodies, maturing modalities such as oligonucleotides, and emerging modalities such as gene therapy and gene editing. The choice of therapeutic modality can depend on several factors including the location of a target (for example, intracellular, extracellular, or secreted), a relevant tissue (for example, liver, brain, or neural tissue) and a relevant indication.

The fibrinogen gamma chain gene, also known as fibrinogen gamma gene (FGG), is located on chromosome 4, and encodes fibrinogen gamma chain (also referred to as FGG protein). The FGG protein may be a gamma component of fibrinogen. FGG protein may include 453 amino acids and have a mass of about 51.5 kDa. An example of a FGG amino acid sequence, and further description of FGG is included at uniprot.org under accession no. P02679 (last modified Sep. 29, 2021).

Here it is shown that genetic variants causing inactivation of FGG resulted in protective associations for psychiatric and neurological phenotypes. Therefore, inhibition of FGG may serve as a therapeutic for treatment of psychiatric diseases and disorders such as depressive disorder (e.g., major depressive disorder, persistent depressive disorder, treatment resistant depression, or signs and symptoms of depression), post-traumatic stress disorder (PTSD), mood disorders, anxiety disorders, eating disorders, substance-use disorders, bipolar disorder, personality disorders, schizophrenia and schizoaffective disorders, and neurological diseases and disorders such as Alzheimer's disease, dementia, delirium, cognitive decline, vascular dementia, headache, migraine, chronic pain, fibromyalgia, chronic fatigue syndrome (e.g. myalgic encephalomyelitis (ME)), or motor neuron disease (e.g., amyotrophic lateral sclerosis).

Disclosed herein are compositions comprising an oligonucleotide that targets FGG. Where inhibition or targeting of FGG is disclosed, it is contemplated that some embodiments may include inhibiting or targeting a FGG protein or FGG RNA. For example, by inhibiting or targeting an RNA (e.g. mRNA) encoded by the FGG gene using an oligonucleotide described herein, the FGG protein may be inhibited or targeted as a result of there being less production of the FGG protein by translation of the FGG RNA; or a FGG protein may be targeted or inhibited by an oligonucleotide that binds or interacts with a FGG RNA and reduces production of the FGG protein from the FGG RNA. Thus, targeting FGG may refer to binding a FGG RNA and reducing FGG RNA or protein levels. The oligonucleotide may include a small interfering RNA (siRNA) or an antisense oligonucleotide (ASO).

Also provided herein are methods of treating a mental disorder, such as a psychiatric disorder or neurological disorder or disease by providing or administering an oligonucleotide that targets FGG to a subject in need thereof. Administration of the oligonucleotide to a subject may improve psychiatric related traits, such as Montgomery-Asberg Depression Rating Scale (MADRS) (e.g. scale ranges from 0 to 60 with a higher score indicating worsening symptoms of depression), Hamilton Depression Rating Scale-17 (e.g. scale ranges from 0 to 52 with a higher score indicating worsening symptoms of depression), anxiety symptoms and signs, eating disorder symptoms and signs, substance-use disorder symptoms and signs, post-traumatic stress disorder symptoms and signs, bipolar disorder symptoms and signs, schizophrenia symptoms and signs, or psychosis symptoms and signs. Additionally, administration of the oligonucleotide to a subject may improve neurological related traits, such as Cognitive function, CNS amyloid plaques (e.g., accumulation), CNS tau accumulation, CSF beta-amyloid 42 (e.g., accumulation), CSF tau (e.g., accumulation), CSF phospho-tau (e.g., accumulation), Lewy bodies (e.g., accumulation), CSF alpha-synuclein (e.g., accumulation), headache symptoms and signs, migraine symptoms and signs, chronic pain symptoms and signs, fibromyalgia symptoms and signs, chronic fatigue syndrome (ME) symptoms and signs, or motor neuron disease (e.g. ALS) symptoms or signs.

### I. Compositions

Disclosed herein, in some embodiments, are compositions comprising an oligonucleotide. In some embodiments, the composition comprises an oligonucleotide that targets FGG. In some embodiments, the composition consists of an oligonucleotide that targets FGG. In some embodiments, the oligonucleotide reduces FGG mRNA expression in the subject. In some embodiments, the oligonucleotide reduces FGG protein expression in the subject. The oligonucleotide may include a small interfering RNA (siRNA) described herein. The oligonucleotide may include an antisense oligonucleotide (ASO) described herein. In some embodiments, a composition described herein is used in a method of treating a disorder in a subject in need thereof. Some embodiments relate to a composition comprising an oligonucleotide for use in a method of treating a disorder as described herein. Some embodiments relate to use of a composition comprising an oligonucleotide, in a method of treating a disorder (e.g., psychiatric or neurological) as described herein.

Some embodiments include a composition comprising an oligonucleotide that targets FGG and when administered to a subject in an effective amount decreases FGG mRNA or protein levels in a cell (e.g. hepatocyte or neuron), fluid (e.g., blood, serum, plasma, or cerebrospinal fluid (CSF)), tissue (e.g. brain or liver tissue), or organ (e.g., the brain or liver).

In some embodiments, the composition comprises an oligonucleotide that targets FGG and when administered to a subject in an effective amount decreases FGG mRNA levels in a cell or tissue. In some embodiments, the cell is a liver cell (e.g., hepatocyte). In some embodiments, the cell is a neuron. In some embodiments, the tissue is liver tissue. In some embodiments, the tissue is neural tissue. In some embodiments, the neural tissue is CNS tissue. In some embodiments, the neural tissue is brain tissue (e.g., neuronal, glia, or endothelial tissue). In some embodiments, the fluid is CSF. In some embodiments, the FGG mRNA levels are decreased by about 2.5% or more, about 5% or more, or about 7.5% or more, as compared to prior to administration. In some embodiments, the FGG mRNA levels are decreased by about 10% or more, as compared to prior to administration. In some embodiments, the FGG mRNA levels are decreased by about 20% or more, about 30% or more, about 40% or more, about 50% or more, about 60% or more, about 70% or more, about 80% or more, about 90% or more, or about 100%, as compared to prior to administration. In some embodiments, the FGG mRNA levels are decreased by no more than about 2.5%, no more than about 5%, or no more than about 7.5%, as compared to prior to administration. In some embodiments, the FGG mRNA levels are decreased by no more than about 10%, as compared to prior to administration. In some embodiments, the FGG mRNA levels are decreased by no more than about 20%, no more than about 30%, no more than about 40%, no more than about 50%, no more than about 60%, no more than about 70%, no more than about 80%, or no more than about 90%, as compared to prior to administration. In some embodiments, the FGG mRNA levels are decreased by 2.5%, 5%, 7.5%, 10%, 15%, 20%, 25%, 30%, 35%, 40%, 45%, 50%, 55%, 60%, 65%, 70%, 75%, 80%, 85%, 90%, 95%, or 100%, or by a range defined by any of the two aforementioned percentages.

In some embodiments, the composition comprises an oligonucleotide that targets FGG and when administered to a subject in an effective amount decreases FGG protein levels in a cell, fluid (e.g., CSF) or tissue. In some embodiments, the cell is a hepatocyte. In some embodiments, the cell is a neural cell (e.g., CNS cell (e.g., brain cell)). In some embodiments, the cell is a neuronal cell. In some embodiments, the cell is a glial cell. In some embodiments, the cell is an endothelial cell. In some embodiments, the tissue is liver tissue. In some embodiments, the tissue is neural (e.g. CNS (e.g., brain)) tissue. In some embodiments, the fluid is CSF. In some embodiments, the FGG protein levels are decreased by about 2.5% or more, about 5% or more, or about 7.5% or more, as compared to prior to administration. In some embodiments, the FGG protein levels are decreased by about 10% or more, as compared to prior to administration. In some embodiments, the FGG protein levels are decreased by about 20% or more, about 30% or more, about 40% or more, about 50% or more, about 60% or more, about 70% or more, about 80% or more, about 90% or more, or about 100%, as compared to prior to administration. In some embodiments, the FGG protein levels are decreased by no more than about 2.5%, no more than about 5%, or no more than about 7.5%, as compared to prior to administration. In some embodiments, the FGG protein levels are decreased by no more than about 10%, as compared to prior to administration. In some embodiments, the FGG protein levels are decreased by no more than about 20%, no more than about 30%, no more than about 40%, no more than about 50%, no more than about 60%, no more than about 70%, no more than about 80%, or no more than about 90%, as compared to prior to administration. In some embodiments, the FGG protein levels are decreased by 2.5%, 5%, 7.5%, 10%, 15%, 20%, 25%, 30%, 35%, 40%, 45%, 50%, 55%, 60%, 65%, 70%, 75%, 80%, 85%, 90%, 95%, or 100%, or by a range defined by any of the two aforementioned percentages.

In some embodiments, the composition comprises an oligonucleotide that targets FGG and when administered to a subject in an effective amount diminishes a mental disorder or disease phenotype, such as a psychiatric disorder or neurological disorder phenotype. A disorder may include a disease. The psychiatric disease or disorder may include depressive disorder (e.g., major depressive disorder, persistent depressive disorder, treatment resistant depression, or signs and symptoms of depression), post-traumatic stress disorder, mood disorders, anxiety disorders, eating disorders, substance-use disorders, bipolar disorder, personality disorders, schizophrenia and schizoaffective disorders. The neurological disease or disorder may include such as Alzheimer's disease, dementia, delirium, cognitive decline, vascular dementia, headache, migraine, chronic pain, fibromyalgia, chronic fatigue syndrome (e.g. myalgic encephalomyelitis (ME)), or motor neuron disease (e.g., amyotrophic lateral sclerosis). For psychiatric/neurological indications, fibrinogen may be lowered enough to have a therapeutic effect on mental disorders but without significantly affecting coagulation parameters such as PT or aPTT.

In some embodiments, the composition comprises an oligonucleotide that targets FGG and when administered to a subject in an effective amount decreases a psychiatric disease phenotype. The psychiatric disease phenotype may include a Montgomery-Asberg Depression Rating Scale (MADRS) score. The psychiatric disease phenotype may include a Hamilton Depression Rating Scale score. The psychiatric disease phenotype may include a sign or symptom of anxiety. The psychiatric disease phenotype may include a sign or symptom of an eating disorder. The psychiatric disease phenotype may include a sign or symptom of a substance-use disorder. The psychiatric disease phenotype may include a sign or symptom of post-traumatic stress disorder. The psychiatric disease phenotype may include a sign or symptom of bipolar disorder. The psychiatric disease phenotype may include a sign or symptom of schizophrenia. The psychiatric disease phenotype may include a sign or symptom of psychosis. In some embodiments, the psychiatric disease phenotype is decreased by about 2.5% or more, about 5% or more, or about 7.5% or more, as compared to prior to administration. In some embodiments, the psychiatric disease phenotype is decreased by about 10% or more, as compared to prior to administration. In some embodiments, the psychiatric disease phenotype is decreased by about 20% or more, about 30% or more, about 40% or more, about 50% or more, about 60% or more, about 70% or more, about 80% or more, about 90% or more, or about 100%, as compared to prior to administration. In some embodiments, the psychiatric disease phenotype is decreased by no more than about 2.5%, no more than about 5%, or no more than about 7.5%, as compared to prior to administration. In some embodiments, the psychiatric disease phenotype is decreased by no more than about 10%, as compared to prior to administration. In some embodiments, the psychiatric disease phenotype is decreased by no more than about 20%, no more than about 30%, no more than about 40%, no more than about 50%, no more than about 60%, no more than about 70%, no more than about 80%, or no more than about 90%, as compared to prior to administration. In some embodiments, the psychiatric disease phenotype is decreased by 2.5%, 5%, 7.5%, 10%, 15%, 20%, 25%, 30%, 35%, 40%, 45%, 50%, 55%, 60%, 65%, 70%, 75%, 80%, 85%, 90%, 95%, or 100%, or by a range defined by any of the two aforementioned percentages.

In some embodiments, the composition comprises an oligonucleotide that targets FGG and when administered to a subject in an effective amount decreases a neurological disease phenotype. The neurological disease phenotype may include cognitive dysfunction. The neurological disease phenotype may include central nervous system (CNS) amyloid plaques. The neurological disease phenotype may include CNS tau accumulation. The neurological disease phenotype may include cerebrospinal fluid (CSF) beta-amyloid 42. The neurological disease phenotype may include CSF tau. The neurological disease phenotype may include CSF phospho-tau. The neurological disease phenotype may include Lewy bodies. The neurological disease phenotype may include CSF alpha-synuclein. The neurological disease phenotype may include headache symptoms or signs. The neurological disease phenotype may include migraine symptoms or signs. The neurological disease phenotype may include chronic pain symptoms or signs. The neurological disease phenotype may include fibromyalgia symptoms or signs. The neurological disease phenotype may include chronic fatigue syndrome (e.g. myalgic encephalomyelitis) symptoms or signs. The neurological disease phenotype may include motor neuron disease (e.g., amyotrophic lateral sclerosis) symptoms or signs. In some embodiments, the neurological disease phenotype is decreased by about 2.5% or more, about 5% or more, or about 7.5% or more, as compared to prior to administration. In some embodiments, the neurological disease phenotype is decreased by about 10% or more, as compared to prior to administration. In some embodiments, the neurological disease phenotype is decreased by about 20% or more, about 30% or more, about 40% or more, about 50% or more, about 60% or more, about 70% or more, about 80% or more, about 90% or more, or about 100%, as compared to prior to administration. In some embodiments, the neurological disease phenotype is decreased by no more than about 2.5%, no more than about 5%, or no more than about 7.5%, as compared to prior to administration. In some embodiments, the neurological disease phenotype is decreased by no more than about 10%, as compared to prior to administration. In some embodiments, the neurological disease phenotype is decreased by no more than about 20%, no more than about 30%, no more than about 40%, no more than about 50%, no more than about 60%, no more than about 70%, no more than about 80%, or no more than about 90%, as compared to prior to administration. In some embodiments, the neurological disease phenotype is decreased by 2.5%, 5%, 7.5%, 10%, 15%, 20%, 25%, 30%, 35%, 40%, 45%, 50%, 55%, 60%, 65%, 70%, 75%, 80%, 85%, 90%, 95%, or 100%, or by a range defined by any of the two aforementioned percentages.

The composition may treat a clotting or coagulation disorder. The composition may treat thrombophilia. The composition may affect clotting or a clotting time. In some embodiments, the composition comprises an oligonucleotide that decreases Fibrinogen. In some cases, a FGG siRNA composition may be useful as an anticoagulant, such as for treatment or prophylaxis of a coagulation or clotting disorders (e.g. venous thromboembolism, atrial fibrillation), given that significant FGG knockdown may lead to a prolonged clotting time (e.g. PT, INR or aPTT). For coagulation or clotting disorders, it is useful to lower Fibrinogen significantly enough to prolong clotting times to clinically meaningful levels for these indications. Provided herein are data that show FGG siRNA administration may result in FGG knockdown. FGG knockdown may result in decreased circulating fibrinogen. Decreased circulating fibrinogen may result in increased PT, INR and aPTT. As such, the compounds may be useful for reducing clotting. Some aspects relate to a composition comprising an oligonucleotide that targets FGG and when administered to a subject in an effective amount decreases fibrinogen.

In some embodiments, the prothrombin time (PT), International Normalized Ration (INR) and activated partial thromboplastin time (aPTT) levels are unchanged as compared to administration. In some embodiments, PT, INR or aPTT increases by no more than about 10%, as compared to prior to administration. In some embodiments, PT, INR or aPTT increase by no more than about 20%, no more than about 40%, no more than about 80%, no more than about 160%, no more than about 200%, no more than about 300%, no more than about 400%, or no more than about 600%, as compared to prior to administration. In some embodiments, the PT, INR or aPTT increases by 5%, 10%, 20%, 40%, 80%, 100%, 200%, 400% or 600%, or by a range defined by any of the two aforementioned percentages.

A. siRNAs

In some embodiments, the composition comprises an oligonucleotide that targets FGG, wherein the oligonucleotide comprises a small interfering RNA (siRNA). In some embodiments, the composition comprises an oligonucleotide that targets FGG, wherein the oligonucleotide comprises a small interfering RNA (siRNA) comprising a sense strand and an antisense strand.

In some embodiments, the composition comprises an oligonucleotide that inhibits the expression of FGG, wherein the oligonucleotide comprises an siRNA comprising a sense strand and an antisense strand, wherein the sense strand is 12-30 nucleosides in length. In some embodiments, the composition comprises a sense strange that is 10, 11, 12, 13, 14, 15, 16, 17, 18, 19, 20, 21, 22, 23, 24, 25, 26, 27, 28, 29, or 30 nucleosides in length, or a range defined by any of the two aforementioned numbers. The sense strand may be 14-30 nucleosides in length. In some embodiments, the composition comprises an antisense strand is 12-30 nucleosides in length. In some embodiments, the composition comprises an antisense strand that is 10, 11, 12, 13, 14, 15, 16, 17, 18, 19, 20, 21, 22, 23, 24, 25, 26, 27, 28, 29, or 30 nucleosides in length, or a range defined by any of the two aforementioned numbers. The antisense strand may be 14-30 nucleosides in length.

In some embodiments, the composition comprises an oligonucleotide that inhibits the expression of FGG, wherein the oligonucleotide comprises an siRNA comprising a sense strand and an antisense strand, each strand is independently about 12-30 nucleosides in length, and at least one of the sense strand and the antisense strand comprises a nucleoside sequence comprising about 12-30 contiguous nucleosides of a full-length human FGG mRNA sequence such as SEQ ID NO: 3621. In SEQ ID NO: 3621, thymine (T) may be replaced with Uracil (U). In some embodiments, at least one of the sense strand and the antisense strand comprise a nucleoside sequence comprising at least about 10, 11, 12, 13, 14, 15, 16, 17, 18, 19, 20, 21, 22, 23, 24, 25, 26, 27, 28, 29, 30, or more contiguous nucleosides of one of SEQ ID NO: 3621.

In some embodiments, the composition comprises an oligonucleotide that inhibits the expression of FGG, wherein the oligonucleotide comprises an siRNA comprising a sense strand and an antisense strand, wherein the sense strand and the antisense strand form a double-stranded RNA duplex. In some embodiments, the first base pair of the double-stranded RNA duplex is an AU base pair.

In some embodiments, the sense strand further comprises a 3′ overhang. In some embodiments, the 3′ overhang comprises 1, 2, 3, 4, 5, 6, 7, 8, 9, or 10 nucleosides, or a range of nucleotides defined by any two of the aforementioned numbers. In some embodiments, the 3′ overhang comprises 1, 2, or more nucleosides. In some embodiments, the 3′ overhang comprises 2 nucleosides. In some embodiments, the sense strand further comprises a 5′ overhang. In some embodiments, the 5′ overhang comprises 1, 2, 3, 4, 5, 6, 7, 8, 9, or 10 nucleosides, or a range of nucleotides defined by any two of the aforementioned numbers. In some embodiments, the 5′ overhang comprises 1, 2, or more nucleosides. In some embodiments, the 5′ overhang comprises 2 nucleosides.

In some embodiments, the antisense strand further comprises a 3′ overhang. In some embodiments, the 3′ overhang comprises 1, 2, 3, 4, 5, 6, 7, 8, 9, or 10 nucleosides, or a range of nucleotides defined by any two of the aforementioned numbers. In some embodiments, the 3′ overhang comprises 1, 2, or more nucleosides. In some embodiments, the 3′ overhang comprises 2 nucleosides. In some embodiments, the antisense strand further comprises a 5′ overhang. In some embodiments, the 5′ overhang comprises 1, 2, 3, 4, 5, 6, 7, 8, 9, or 10 nucleosides, or a range of nucleotides defined by any two of the aforementioned numbers. In some embodiments, the 5′ overhang comprises 1, 2, or more nucleosides. In some embodiments, the 5′ overhang comprises 2 nucleosides.

In some embodiments, the composition comprises an oligonucleotide that inhibits the expression of FGG, wherein the oligonucleotide comprises an siRNA comprising a sense strand and an antisense strand, wherein the siRNA binds with a 19mer in a human FGG mRNA. In some embodiments, the siRNA binds with a 12mer, a 13mer, a 14mer, a 15mer, a 16mer, a 17mer, a 18mer, a 19mer, a 20mer, a 21mer, a 22mer, a 23mer, a 24mer, or a 25mer in a human FGG mRNA.

In some embodiments, the composition comprises an oligonucleotide that inhibits the expression of FGG, wherein the oligonucleotide comprises an siRNA comprising a sense strand and an antisense strand, wherein the siRNA binds with a 17mer in a non-human primate FGG mRNA. In some embodiments, the siRNA binds with a 12mer, a 13mer, a 14mer, a 15mer, a 16mer, a 17mer, a 18mer, a 19mer, a 20mer, a 21mer, a 22mer, a 23mer, a 24mer, or a 25mer in a non-human primate FGG mRNA.

In some embodiments, the composition comprises an oligonucleotide that inhibits the expression of FGG, wherein the oligonucleotide comprises an siRNA comprising a sense strand and an antisense strand, wherein the siRNA binds with a human FGG mRNA and less than or equal to 20 human off-targets, with no more than 2 mismatches in the antisense strand. In some embodiments, the siRNA binds with a human FGG mRNA and less than or equal to 10 human off-targets, with no more than 2 mismatches in the antisense strand. In some embodiments, the siRNA binds with a human FGG mRNA and less than or equal to 30 human off-targets, with no more than 2 mismatches in the antisense strand. In some embodiments, the siRNA binds with a human FGG mRNA and less than or equal to 40 human off-targets, with no more than 2 mismatches in the antisense strand. In some embodiments, the siRNA binds with a human FGG mRNA and less than or equal to 50 human off-targets, with no more than 2 mismatches in the antisense strand. In some embodiments, the siRNA binds with a human FGG mRNA and less than or equal to 10 human off-targets, with no more than 3 mismatches in the antisense strand. In some embodiments, the siRNA binds with a human FGG mRNA and less than or equal to 20 human off-targets, with no more than 3 mismatches in the antisense strand. In some embodiments, the siRNA binds with a human FGG mRNA and less than or equal to 30 human off-targets, with no more than 3 mismatches in the antisense strand. In some embodiments, the siRNA binds with a human FGG mRNA and less than or equal to 40 human off-targets, with no more than 3 mismatches in the antisense strand. In some embodiments, the siRNA binds with a human FGG mRNA and less than or equal to 50 human off-targets, with no more than 3 mismatches in the antisense strand.

In some embodiments, the composition comprises an oligonucleotide that inhibits the expression of FGG, wherein the oligonucleotide comprises an siRNA comprising a sense strand and an antisense strand, siRNA binds with a human FGG mRNA target site that does not harbor an SNP, with a minor allele frequency (MAF) greater or equal to 1% (pos. 2-18). In some embodiments, the MAF is greater or equal to about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20%.

In some embodiments, the composition comprises an oligonucleotide that inhibits the expression of FGG, wherein the oligonucleotide comprises an siRNA comprising a sense strand and an antisense strand, wherein the sense strand comprises a nucleoside sequence comprising or consisting of the sequence of any one of SEQ ID NOs: 1-1742, or a nucleic acid sequence thereof having 1 or 2 nucleoside substitutions, additions, or deletions. In some embodiments, the sense strand comprises a nucleoside sequence comprising or consisting of the sequence of any one of SEQ ID NOs: 1-1742, or a nucleic acid sequence thereof having 3 or 4 nucleoside substitutions, additions, or deletions. In some embodiments, the sense strand further comprises a 3′ overhang. In some embodiments, the 3′ overhang comprises 1, 2, 3, 4, 5, 6, 7, 8, 9, or 10 nucleosides, or a range of nucleotides defined by any two of the aforementioned numbers. In some embodiments, the 3′ overhang comprises 1, 2, or more nucleosides. In some embodiments, the 3′ overhang comprises 2 nucleosides. In some embodiments, the sense strand further comprises a 5′ overhang. In some embodiments, the 5′ overhang comprises 1, 2, 3, 4, 5, 6, 7, 8, 9, or 10 nucleosides, or a range of nucleotides defined by any two of the aforementioned numbers. In some embodiments, the 5′ overhang comprises 1, 2, or more nucleosides. In some embodiments, the 5′ overhang comprises 2 nucleosides. In some embodiments, the sense strand comprises a nucleoside sequence comprising or consisting of the sequence of any one of SEQ ID NOs: 1-1742, or a nucleic acid sequence thereof having 1 or 2 nucleoside additions at the 3′ end. In some embodiments, the composition comprises an oligonucleotide that inhibits the expression of FGG, wherein the oligonucleotide comprises an siRNA comprising a sense strand and an antisense strand, wherein the sense strand comprises a nucleoside sequence comprising or consisting of the sequence of any one of SEQ ID NOs: 1-1742. In any of SEQ ID NOs: 1-1742, thymine (T) may be replaced with Uracil (U).

In some embodiments, the composition comprises an oligonucleotide that inhibits the expression of FGG, wherein the oligonucleotide comprises an siRNA comprising a sense strand and an antisense strand, wherein the antisense strand comprises a nucleoside sequence comprising or consisting of the sequence of any one of SEQ ID NOs: 1743-3484, or a nucleic acid sequence thereof having 1 or 2 nucleoside substitutions, additions, or deletions. In some embodiments, the antisense strand sequence comprises a nucleoside sequence comprising or consisting of the sequence of any one of SEQ ID NOs: 1743-3484 or a nucleic acid sequence thereof having 3 or 4 nucleoside substitutions, additions, or deletions. In some embodiments, the antisense strand further comprises a 3′ overhang. In some embodiments, the 3′ overhang comprises 1, 2, 3, 4, 5, 6, 7, 8, 9, or 10 nucleosides, or a range of nucleotides defined by any two of the aforementioned numbers. In some embodiments, the 3′ overhang comprises 1, 2, or more nucleosides. In some embodiments, the 3′ overhang comprises 2 nucleosides. In some embodiments, the antisense strand further comprises a 5′ overhang. In some embodiments, the 5′ overhang comprises 1, 2, 3, 4, 5, 6, 7, 8, 9, or 10 nucleosides, or a range of nucleotides defined by any two of the aforementioned numbers. In some embodiments, the 5′ overhang comprises 1, 2, or more nucleosides. In some embodiments, the 5′ overhang comprises 2 nucleosides. In some embodiments, the antisense strand comprises a nucleoside sequence comprising or consisting of the sequence of any one of SEQ ID NOs: 1743-3484, or a nucleic acid sequence thereof having 1 or 2 nucleoside additions at the 3′ end. In some embodiments, the composition comprises an oligonucleotide that inhibits the expression of FGG, wherein the oligonucleotide comprises an siRNA comprising a sense strand and an antisense strand, wherein the antisense strand comprises a nucleoside sequence comprising or consisting of the sequence of any one of SEQ ID NOs: 1743-3484. In any of SEQ ID NOs: 1743-3484, thymine (T) may be replaced with Uracil (U).

In some embodiments, the composition comprises an oligonucleotide that inhibits the expression of FGG, wherein the oligonucleotide comprises an siRNA comprising a sense strand and an antisense strand, wherein the sense strand comprises a nucleoside sequence comprising or consisting of the sequence of any one of SEQ ID NOs: 3713-3748, or a nucleic acid sequence thereof having 1 or 2 nucleoside substitutions, additions, or deletions. In some embodiments, the sense strand comprises a nucleoside sequence comprising or consisting of the sequence of any one of SEQ ID NOs: 3713-3748, or a nucleic acid sequence thereof having 3 or 4 nucleoside substitutions, additions, or deletions. In some embodiments, the sense strand further comprises a 3′ overhang. In some embodiments, the 3′ overhang comprises 1, 2, 3, 4, 5, 6, 7, 8, 9, or 10 nucleosides, or a range of nucleotides defined by any two of the aforementioned numbers. In some embodiments, the 3′ overhang comprises 1, 2, or more nucleosides. In some embodiments, the 3′ overhang comprises 2 nucleosides. In some embodiments, the sense strand further comprises a 5′ overhang. In some embodiments, the 5′ overhang comprises 1, 2, 3, 4, 5, 6, 7, 8, 9, or 10 nucleosides, or a range of nucleotides defined by any two of the aforementioned numbers. In some embodiments, the 5′ overhang comprises 1, 2, or more nucleosides. In some embodiments, the 5′ overhang comprises 2 nucleosides. In some embodiments, the sense strand comprises a nucleoside sequence comprising or consisting of the sequence of any one of SEQ ID NOs: 3713-3748, or a nucleic acid sequence thereof having 1 or 2 nucleoside additions at the 3′ end. In some embodiments, the composition comprises an oligonucleotide that inhibits the expression of FGG, wherein the oligonucleotide comprises an siRNA comprising a sense strand and an antisense strand, wherein the sense strand comprises a nucleoside sequence comprising or consisting of the sequence of any one of SEQ ID NOs: 3713-3748. In any of SEQ ID NOs: 3713-3748, thymine (T) may be replaced with Uracil (U).

In some embodiments, the composition comprises an oligonucleotide that inhibits the expression of FGG, wherein the oligonucleotide comprises an siRNA comprising a sense strand and an antisense strand, wherein the antisense strand comprises a nucleoside sequence comprising or consisting of the sequence of any one of SEQ ID NOs: 3749-3784, or a nucleic acid sequence thereof having 1 or 2 nucleoside substitutions, additions, or deletions. In some embodiments, the antisense strand sequence comprises a nucleoside sequence comprising or consisting of the sequence of any one of SEQ ID NOs: 3749-3784 or a nucleic acid sequence thereof having 3 or 4 nucleoside substitutions, additions, or deletions. In some embodiments, the antisense strand further comprises a 3′ overhang. In some embodiments, the 3′ overhang comprises 1, 2, 3, 4, 5, 6, 7, 8, 9, or 10 nucleosides, or a range of nucleotides defined by any two of the aforementioned numbers. In some embodiments, the 3′ overhang comprises 1, 2, or more nucleosides. In some embodiments, the 3′ overhang comprises 2 nucleosides. In some embodiments, the antisense strand further comprises a 5′ overhang. In some embodiments, the 5′ overhang comprises 1, 2, 3, 4, 5, 6, 7, 8, 9, or 10 nucleosides, or a range of nucleotides defined by any two of the aforementioned numbers. In some embodiments, the 5′ overhang comprises 1, 2, or more nucleosides. In some embodiments, the 5′ overhang comprises 2 nucleosides. In some embodiments, the antisense strand comprises a nucleoside sequence comprising or consisting of the sequence of any one of SEQ ID NOs: 3749-3784, or a nucleic acid sequence thereof having 1 or 2 nucleoside additions at the 3′ end. In some embodiments, the composition comprises an oligonucleotide that inhibits the expression of FGG, wherein the oligonucleotide comprises an siRNA comprising a sense strand and an antisense strand, wherein the antisense strand comprises a nucleoside sequence comprising or consisting of the sequence of any one of SEQ ID NOs: 3749-3784. In any of SEQ ID NOs: 3749-3784, thymine (T) may be replaced with Uracil (U).

In some embodiments, the sense and/or antisense strand comprises a nucleoside sequence at least 70% identical, at least 75% identical, at least 80% identical, at least 85% identical, at least 90% identical, or at least 95% identical, to a sense and/or antisense strand sequence in any of Tables 3-7. In some embodiments, the siRNA comprises the sense strand and/or the antisense strand sequence of an siRNA in any Tables 3-7, or a nucleic acid sequence thereof having 3 or 4 nucleoside substitutions, additions, or deletions. In some embodiments, the siRNA comprises the sense strand and/or the antisense strand sequence of an siRNA in any Tables 3-7, or a nucleic acid sequence thereof having 1 or 2 nucleoside substitutions, additions, or deletions. In some embodiments, the siRNA comprises the sense strand and/or the antisense strand sequence of an siRNA in any Tables 3-7. In some embodiments, the siRNA is cross-reactive with a non-human primate (NHP) FGG mRNA. The siRNA may include one or more internucleoside linkages and/or one or more nucleoside modifications.

In some embodiments, the siRNA comprises the sense strand and/or the antisense strand sequence of an siRNA of subset A, or a nucleic acid sequence thereof having 3 or 4 nucleoside substitutions, additions, or deletions. In some embodiments, the siRNA comprises the sense strand and/or the antisense strand sequence of an siRNA of subset A, or a nucleic acid sequence thereof having 1 or 2 nucleoside substitutions, additions, or deletions. In some embodiments, the siRNA comprises the sense strand and/or the antisense strand sequence of an siRNA of subset A. The siRNA may include one or more internucleoside linkages and/or one or more nucleoside modifications.

In some embodiments, the siRNA comprises the sense strand and/or the antisense strand sequence of an siRNA of subset B, or a nucleic acid sequence thereof having 3 or 4 nucleoside substitutions, additions, or deletions. In some embodiments, the siRNA comprises the sense strand and/or the antisense strand sequence of an siRNA of subset B, or a nucleic acid sequence thereof having 1 or 2 nucleoside substitutions, additions, or deletions. In some embodiments, the siRNA comprises the sense strand and/or the antisense strand sequence of an siRNA of subset B. The siRNA may include one or more internucleoside linkages and/or one or more nucleoside modifications.

In some embodiments, the siRNA comprises the sense strand and/or the antisense strand sequence of an siRNA of subset C, or a nucleic acid sequence thereof having 3 or 4 nucleoside substitutions, additions, or deletions. In some embodiments, the siRNA comprises the sense strand and/or the antisense strand sequence of an siRNA of subset C, or a nucleic acid sequence thereof having 1 or 2 nucleoside substitutions, additions, or deletions. In some embodiments, the siRNA comprises the sense strand and/or the antisense strand sequence of an siRNA of subset C. The siRNA may include one or more internucleoside linkages and/or one or more nucleoside modifications.

In some embodiments, the siRNA comprises the sense strand and/or the antisense strand sequence of an siRNA of subset D, or a nucleic acid sequence thereof having 3 or 4 nucleoside substitutions, additions, or deletions. In some embodiments, the siRNA comprises the sense strand and/or the antisense strand sequence of an siRNA of subset D, or a nucleic acid sequence thereof having 1 or 2 nucleoside substitutions, additions, or deletions. In some embodiments, the siRNA comprises the sense strand and/or the antisense strand sequence of an siRNA of subset D. The siRNA may include one or more internucleoside linkages and/or one or more nucleoside modifications.

In some embodiments, the siRNA comprises the sense strand and/or the antisense strand sequence of an siRNA of subset E, or a nucleic acid sequence thereof having 3 or 4 nucleoside substitutions, additions, or deletions. In some embodiments, the siRNA comprises the sense strand and/or the antisense strand sequence of an siRNA of subset E, or a nucleic acid sequence thereof having 1 or 2 nucleoside substitutions, additions, or deletions. In some embodiments, the siRNA comprises the sense strand and/or the antisense strand sequence of an siRNA of subset E. The siRNA may include one or more internucleoside linkages and/or one or more nucleoside modifications.

In some embodiments, the siRNA comprises the sense strand and/or the antisense strand sequence of an siRNA of subset G, or a nucleic acid sequence thereof having 3 or 4 nucleoside substitutions, additions, or deletions. In some embodiments, the siRNA comprises the sense strand and/or the antisense strand sequence of an siRNA of subset G, or a nucleic acid sequence thereof having 1 or 2 nucleoside substitutions, additions, or deletions. In some embodiments, the siRNA comprises the sense strand and/or the antisense strand sequence of an siRNA of subset G. The siRNA may include one or more internucleoside linkages and/or one or more nucleoside modifications.

In some embodiments, the sense strand comprises a nucleoside sequence at least 70% identical, at least 75% identical, at least 80% identical, at least 85% identical, at least 90% identical, or at least 95% identical, to any one of SEQ ID NOs: 352, 1003, 1011, or 1278. In some embodiments, the sense strand comprises the nucleoside sequence of any one of SEQ ID NOS: 352, 1003, 1011, or 1278, or a sense strand sequence thereof having 1 or 2 nucleoside substitutions, additions, or deletions. In some embodiments, the sense strand comprises the nucleoside sequence of any one of SEQ ID NOS: 352, 1003, 1011, or 1278, and 3 or 4 nucleoside substitutions, additions, or deletions. In some embodiments, the sense strand comprises the nucleoside sequence of any one of SEQ ID NOS: 352, 1003, 1011, or 1278. The sense strand may include any internucleoside linkages or nucleoside modifications described herein. The sense strand may include an overhang (e.g. 2 bases on a 5 or 3′ end when paired with an antisense strand). The sense strand may include a GalNAc moiety connected at one of the ends (e.g. 5′ end).

In some embodiments, the sense strand comprises a nucleoside sequence at least 85% identical to SEQ ID NO: 352. In some embodiments, the sense strand comprises the nucleoside sequence of SEQ ID NO: 352, or a sense strand sequence thereof having 1 or 2 nucleoside substitutions, additions, or deletions. In some embodiments, the sense strand comprises the nucleoside sequence of SEQ ID NO: 352, and 3 or 4 nucleoside substitutions, additions, or deletions. In some embodiments, the sense strand comprises the nucleoside sequence of SEQ ID NO: 352. The sense strand may include any internucleoside linkages or nucleoside modifications described herein. The sense strand may include an overhang (e.g. 2 bases on a 5 or 3′ end when paired with an antisense strand). The sense strand may include a GalNAc moiety connected at one of the ends (e.g. 5′ end).

In some embodiments, the sense strand comprises a nucleoside sequence at least 85% identical to SEQ ID NO: 1003. In some embodiments, the sense strand comprises the nucleoside sequence of SEQ ID NO: 1003, or a sense strand sequence thereof having 1 or 2 nucleoside substitutions, additions, or deletions. In some embodiments, the sense strand comprises the nucleoside sequence of SEQ ID NO: 1003, and 3 or 4 nucleoside substitutions, additions, or deletions. In some embodiments, the sense strand comprises the nucleoside sequence of SEQ ID NO: 1003. The sense strand may include any internucleoside linkages or nucleoside modifications described herein. The sense strand may include an overhang (e.g. 2 bases on a 5 or 3′ end when paired with an antisense strand). The sense strand may include a GalNAc moiety connected at one of the ends (e.g. 5′ end).

In some embodiments, the sense strand comprises a nucleoside sequence at least 85% identical to SEQ ID NO: 1011. In some embodiments, the sense strand comprises the nucleoside sequence of SEQ ID NO: 1011, or a sense strand sequence thereof having 1 or 2 nucleoside substitutions, additions, or deletions. In some embodiments, the sense strand comprises the nucleoside sequence of SEQ ID NO: 1011, and 3 or 4 nucleoside substitutions, additions, or deletions. In some embodiments, the sense strand comprises the nucleoside sequence of SEQ ID NO: 1011. The sense strand may include any internucleoside linkages or nucleoside modifications described herein. The sense strand may include an overhang (e.g. 2 bases on a 5 or 3′ end when paired with an antisense strand). The sense strand may include a GalNAc moiety connected at one of the ends (e.g. 5′ end).

In some embodiments, the sense strand comprises a nucleoside sequence at least 85% identical to SEQ ID NO: 1278. In some embodiments, the sense strand comprises the nucleoside sequence of SEQ ID NO: 1278, or a sense strand sequence thereof having 1 or 2 nucleoside substitutions, additions, or deletions. In some embodiments, the sense strand comprises the nucleoside sequence of SEQ ID NO: 1278, and 3 or 4 nucleoside substitutions, additions, or deletions. In some embodiments, the sense strand comprises the nucleoside sequence of SEQ ID NO: 1278. The sense strand may include any internucleoside linkages or nucleoside modifications described herein. The sense strand may include an overhang (e.g. 2 bases on a 5 or 3′ end when paired with an antisense strand). The sense strand may include a GalNAc moiety connected at one of the ends (e.g. 5′ end).

In some embodiments, the antisense strand comprises a nucleoside sequence at least 70% identical, at least 75% identical, at least 80% identical, at least 85% identical, at least 90% identical, or at least 95% identical, to any one of SEQ ID NOs: 2094, 2745, 2753, or 3020. In some embodiments, the antisense strand comprises the nucleoside sequence of any one of SEQ ID NOS: 2094, 2745, 2753, or 3020, or an antisense strand sequence thereof having 1 or 2 nucleoside substitutions, additions, or deletions. In some embodiments, the antisense strand comprises the nucleoside sequence of any one of SEQ ID NOS: 2094, 2745, 2753, or 3020, and 3 or 4 nucleoside substitutions, additions, or deletions. In some embodiments, the antisense strand comprises the nucleoside sequence of any one of SEQ ID NOS: 2094, 2745, 2753, or 3020. The antisense strand may include any internucleoside linkages or nucleoside modifications described herein. The antisense strand may include an overhang (e.g. 2 bases on a 5 or 3′ end when paired with a sense strand). The antisense strand may include a GalNAc moiety connected at one of the ends (e.g. 5′ end or 3′ end).

In some embodiments, the antisense strand comprises a nucleoside sequence at least 85% identical to SEQ ID NO: 2094. In some embodiments, the antisense strand comprises the nucleoside sequence of SEQ ID NO: 2094, or an antisense strand sequence thereof having 1 or 2 nucleoside substitutions, additions, or deletions. In some embodiments, the antisense strand comprises the nucleoside sequence of SEQ ID NO: 2094, and 3 or 4 nucleoside substitutions, additions, or deletions. In some embodiments, the antisense strand comprises the nucleoside sequence of SEQ ID NO: 2094. The antisense strand may include any internucleoside linkages or nucleoside modifications described herein. The antisense strand may include an overhang (e.g. 2 bases on a 5 or 3′ end when paired with a sense strand). The sense strand may include a GalNAc moiety connected at one of the ends.

In some embodiments, the antisense strand comprises a nucleoside sequence at least 85% identical to SEQ ID NO: 2745. In some embodiments, the antisense strand comprises the nucleoside sequence of SEQ ID NO: 2745, or an antisense strand sequence thereof having 1 or 2 nucleoside substitutions, additions, or deletions. In some embodiments, the antisense strand comprises the nucleoside sequence of SEQ ID NO: 2745, and 3 or 4 nucleoside substitutions, additions, or deletions. In some embodiments, the antisense strand comprises the nucleoside sequence of SEQ ID NO: 2745. The antisense strand may include any internucleoside linkages or nucleoside modifications described herein. The antisense strand may include an overhang (e.g. 2 bases on a 5 or 3′ end when paired with a sense strand). The sense strand may include a GalNAc moiety connected at one of the ends.

In some embodiments, the antisense strand comprises a nucleoside sequence at least 85% identical to SEQ ID NO: 2753. In some embodiments, the antisense strand comprises the nucleoside sequence of SEQ ID NO: 2753, or an antisense strand sequence thereof having 1 or 2 nucleoside substitutions, additions, or deletions. In some embodiments, the antisense strand comprises the nucleoside sequence of SEQ ID NO: 2753, and 3 or 4 nucleoside substitutions, additions, or deletions. In some embodiments, the antisense strand comprises the nucleoside sequence of SEQ ID NO: 2753. The antisense strand may include any internucleoside linkages or nucleoside modifications described herein. The antisense strand may include an overhang (e.g. 2 bases on a 5 or 3′ end when paired with a sense strand). The sense strand may include a GalNAc moiety connected at one of the ends.

In some embodiments, the antisense strand comprises a nucleoside sequence at least 85% identical to SEQ ID NO: 3020. In some embodiments, the antisense strand comprises the nucleoside sequence of SEQ ID NO: 3020, or an antisense strand sequence thereof having 1 or 2 nucleoside substitutions, additions, or deletions. In some embodiments, the antisense strand comprises the nucleoside sequence of SEQ ID NO: 3020, and 3 or 4 nucleoside substitutions, additions, or deletions. In some embodiments, the antisense strand comprises the nucleoside sequence of SEQ ID NO: 3020. The antisense strand may include any internucleoside linkages or nucleoside modifications described herein. The antisense strand may include an overhang (e.g. 2 bases on a 5 or 3′ end when paired with a sense strand). The sense strand may include a GalNAc moiety connected at one of the ends.

In some embodiments, the sense strand comprises a nucleoside sequence at least 70% identical, at least 75% identical, at least 80% identical, at least 85% identical, at least 90% identical, or at least 95% identical, to any one of SEQ ID NOs: 3723, 3724, 3726, or 3747. In some embodiments, the sense strand comprises the nucleoside sequence of any one of SEQ ID NOS: 3723, 3724, 3726, or 3747, or a sense strand sequence thereof having 1 or 2 nucleoside substitutions, additions, or deletions. In some embodiments, the sense strand comprises the nucleoside sequence of any one of SEQ ID NOS: 3723, 3724, 3726, or 3747, and 3 or 4 nucleoside substitutions, additions, or deletions. In some embodiments, the sense strand comprises the nucleoside sequence of any one of SEQ ID NOS: 3723, 3724, 3726, or 3747. The sense strand may include any internucleoside linkages or nucleoside modifications described herein. The sense strand may include an overhang (e.g. 2 bases on a 5 or 3′ end when paired with an antisense strand). The sense strand may include a GalNAc moiety connected at one of the ends (e.g. 5′ end).

In some embodiments, the sense strand comprises a nucleoside sequence at least 85% identical to SEQ ID NO: 3723. In some embodiments, the sense strand comprises the nucleoside sequence of SEQ ID NO: 3723, or a sense strand sequence thereof having 1 or 2 nucleoside substitutions, additions, or deletions. In some embodiments, the sense strand comprises the nucleoside sequence of SEQ ID NO: 3723, and 3 or 4 nucleoside substitutions, additions, or deletions. In some embodiments, the sense strand comprises the nucleoside sequence of SEQ ID NO: 3723. The sense strand may include any internucleoside linkages or nucleoside modifications described herein. The sense strand may include an overhang (e.g. 2 bases on a 5 or 3′ end when paired with an antisense strand). The sense strand may include a GalNAc moiety connected at one of the ends (e.g. 5′ end).

In some embodiments, the sense strand comprises a nucleoside sequence at least 85% identical to SEQ ID NO: 3724. In some embodiments, the sense strand comprises the nucleoside sequence of SEQ ID NO: 3724, or a sense strand sequence thereof having 1 or 2 nucleoside substitutions, additions, or deletions. In some embodiments, the sense strand comprises the nucleoside sequence of SEQ ID NO: 3724, and 3 or 4 nucleoside substitutions, additions, or deletions. In some embodiments, the sense strand comprises the nucleoside sequence of SEQ ID NO: 3724. The sense strand may include any internucleoside linkages or nucleoside modifications described herein. The sense strand may include an overhang (e.g. 2 bases on a 5 or 3′ end when paired with an antisense strand). The sense strand may include a GalNAc moiety connected at one of the ends (e.g. 5′ end).

In some embodiments, the sense strand comprises a nucleoside sequence at least 85% identical to SEQ ID NO: 3726. In some embodiments, the sense strand comprises the nucleoside sequence of SEQ ID NO: 3726, or a sense strand sequence thereof having 1 or 2 nucleoside substitutions, additions, or deletions. In some embodiments, the sense strand comprises the nucleoside sequence of SEQ ID NO: 3726, and 3 or 4 nucleoside substitutions, additions, or deletions. In some embodiments, the sense strand comprises the nucleoside sequence of SEQ ID NO: 3726. The sense strand may include any internucleoside linkages or nucleoside modifications described herein. The sense strand may include an overhang (e.g. 2 bases on a 5 or 3′ end when paired with an antisense strand). The sense strand may include a GalNAc moiety connected at one of the ends (e.g. 5′ end).

In some embodiments, the sense strand comprises a nucleoside sequence at least 85% identical to SEQ ID NO: 3747. In some embodiments, the sense strand comprises the nucleoside sequence of SEQ ID NO: 3747, or a sense strand sequence thereof having 1 or 2 nucleoside substitutions, additions, or deletions. In some embodiments, the sense strand comprises the nucleoside sequence of SEQ ID NO: 3747, and 3 or 4 nucleoside substitutions, additions, or deletions. In some embodiments, the sense strand comprises the nucleoside sequence of SEQ ID NO: 3747. The sense strand may include any internucleoside linkages or nucleoside modifications described herein. The sense strand may include an overhang (e.g. 2 bases on a 5 or 3′ end when paired with an antisense strand). The sense strand may include a GalNAc moiety connected at one of the ends (e.g. 5′ end).

In some embodiments, the antisense strand comprises a nucleoside sequence at least 70% identical, at least 75% identical, at least 80% identical, at least 85% identical, at least 90% identical, or at least 95% identical, to any one of SEQ ID NOs: 3759, 3760, 3762, or 3783. In some embodiments, the antisense strand comprises the nucleoside sequence of any one of SEQ ID NOS: 3759, 3760, 3762, or 3783, or an antisense strand sequence thereof having 1 or 2 nucleoside substitutions, additions, or deletions. In some embodiments, the antisense strand comprises the nucleoside sequence of any one of SEQ ID NOS: 3759, 3760, 3762, or 3783, and 3 or 4 nucleoside substitutions, additions, or deletions. In some embodiments, the antisense strand comprises the nucleoside sequence of any one of SEQ ID NOS: 3759, 3760, 3762, or 3783. The antisense strand may include any internucleoside linkages or nucleoside modifications described herein. The antisense strand may include an overhang (e.g. 2 bases on a 5 or 3′ end when paired with a sense strand). The antisense strand may include a GalNAc moiety connected at one of the ends (e.g. 5′ end or 3′ end).

In some embodiments, the antisense strand comprises a nucleoside sequence at least 85% identical to SEQ ID NO: 3759. In some embodiments, the antisense strand comprises the nucleoside sequence of SEQ ID NO: 3759, or an antisense strand sequence thereof having 1 or 2 nucleoside substitutions, additions, or deletions. In some embodiments, the antisense strand comprises the nucleoside sequence of SEQ ID NO: 3759, and 3 or 4 nucleoside substitutions, additions, or deletions. In some embodiments, the antisense strand comprises the nucleoside sequence of SEQ ID NO: 3759. The antisense strand may include any internucleoside linkages or nucleoside modifications described herein. The antisense strand may include an overhang (e.g. 2 bases on a 5 or 3′ end when paired with a sense strand). The sense strand may include a GalNAc moiety connected at one of the ends.

In some embodiments, the antisense strand comprises a nucleoside sequence at least 85% identical to SEQ ID NO: 3760. In some embodiments, the antisense strand comprises the nucleoside sequence of SEQ ID NO: 3760, or an antisense strand sequence thereof having 1 or 2 nucleoside substitutions, additions, or deletions. In some embodiments, the antisense strand comprises the nucleoside sequence of SEQ ID NO: 3760, and 3 or 4 nucleoside substitutions, additions, or deletions. In some embodiments, the antisense strand comprises the nucleoside sequence of SEQ ID NO: 3760. The antisense strand may include any internucleoside linkages or nucleoside modifications described herein. The antisense strand may include an overhang (e.g. 2 bases on a 5 or 3′ end when paired with a sense strand). The sense strand may include a GalNAc moiety connected at one of the ends.

In some embodiments, the antisense strand comprises a nucleoside sequence at least 85% identical to SEQ ID NO: 3762. In some embodiments, the antisense strand comprises the nucleoside sequence of SEQ ID NO: 3762, or an antisense strand sequence thereof having 1 or 2 nucleoside substitutions, additions, or deletions. In some embodiments, the antisense strand comprises the nucleoside sequence of SEQ ID NO: 3762, and 3 or 4 nucleoside substitutions, additions, or deletions. In some embodiments, the antisense strand comprises the nucleoside sequence of SEQ ID NO: 3762. The antisense strand may include any internucleoside linkages or nucleoside modifications described herein. The antisense strand may include an overhang (e.g. 2 bases on a 5 or 3′ end when paired with a sense strand). The sense strand may include a GalNAc moiety connected at one of the ends.

In some embodiments, the antisense strand comprises a nucleoside sequence at least 85% identical to SEQ ID NO: 3783. In some embodiments, the antisense strand comprises the nucleoside sequence of SEQ ID NO: 3783, or an antisense strand sequence thereof having 1 or 2 nucleoside substitutions, additions, or deletions. In some embodiments, the antisense strand comprises the nucleoside sequence of SEQ ID NO: 3783, and 3 or 4 nucleoside substitutions, additions, or deletions. In some embodiments, the antisense strand comprises the nucleoside sequence of SEQ ID NO: 3783. The antisense strand may include any internucleoside linkages or nucleoside modifications described herein. The antisense strand may include an overhang (e.g. 2 bases on a 5 or 3′ end when paired with a sense strand). The sense strand may include a GalNAc moiety connected at one of the ends.

### B. ASOs

In some embodiments, the composition comprises an oligonucleotide that inhibits the expression of FGG, wherein the oligonucleotide comprises an antisense oligonucleotide (ASO). In some embodiments, the ASO is 12-30 nucleosides in length. In some embodiments, the ASO is 14-30 nucleosides in length. In some embodiments, the ASO is at least about 10, 11, 12, 13, 14, 15, 16, 17, 18, 19, 20, 21, 22, 23, 24, 25, 26, 27, 28, 29, or 30 nucleosides in length, or a range defined by any of the two aforementioned numbers. In some embodiments, the ASO is 15-25 nucleosides in length. In some embodiments, the ASO is 20 nucleosides in length.

In some embodiments, the composition comprises an oligonucleotide that inhibits the expression of FGG, wherein the oligonucleotide comprises an ASO about 12-30 nucleosides in length and comprising a nucleoside sequence complementary to about 12-30 contiguous nucleosides of a full-length human FGG mRNA sequence such as SEQ ID NO: 3621; wherein (i) the oligonucleotide comprises a modification comprising a modified nucleoside and/or a modified internucleoside linkage, and/or (ii) the composition comprises a pharmaceutically acceptable carrier. In some embodiments, the ASO comprise a nucleoside sequence complementary to at least about 10, 11, 12, 13, 14, 15, 16, 17, 18, 19, 20, 21, 22, 23, 24, 25, 26, 27, 28, 29, 30, or more contiguous nucleosides of one of SEQ ID NO: 3621.

### C. Modification Patterns

In some embodiments, the composition comprises an oligonucleotide that inhibits the expression of FGG, wherein the oligonucleotide comprises a modification comprising a modified nucleoside and/or a modified internucleoside linkage, and/or (ii) the composition comprises a pharmaceutically acceptable carrier. In some embodiments, the oligonucleotide comprises a modification comprising a modified nucleoside and/or a modified internucleoside linkage. In some embodiments, the oligonucleotide comprises a modified internucleoside linkage. In some embodiments, the modified internucleoside linkage comprises alkylphosphonate, phosphorothioate, methylphosphonate, phosphorodithioate, alkylphosphonothioate, phosphoramidate, carbamate, carbonate, phosphate triester, acetamidate, or carboxymethyl ester, or a combination thereof. In some embodiments, the modified internucleoside linkage comprises one or more phosphorothioate linkages. A phosphorothioate may include a nonbridging oxygen atom in a phosphate backbone of the oligonucleotide that is replaced by sulfur. Modified internucleoside linkages may be included in siRNAs or ASOs. Benefits of the modified internucleoside linkage may include decreased toxicity or improved pharmacokinetics.

In some embodiments, the composition comprises an oligonucleotide that inhibits the expression of FGG, wherein the oligonucleotide comprises a modified internucleoside linkage, wherein the oligonucleotide comprises 1, 2, 3, 4, 5, 6, 7, 8, 9, 10, 11, 12, 13, 14, 15, 16, 17, 18, 19, or 20 modified internucleoside linkages, or a range of modified internucleoside linkages defined by any two of the aforementioned numbers. In some embodiments, the oligonucleotide comprises no more than 18 modified internucleoside linkages. In some embodiments, the oligonucleotide comprises no more than 20 modified internucleoside linkages. In some embodiments, the oligonucleotide comprises 2 or more modified internucleoside linkages, 3 or more modified internucleoside linkages, 4 or more modified internucleoside linkages, 5 or more modified internucleoside linkages, 6 or more modified internucleoside linkages, 7 or more modified internucleoside linkages, 8 or more modified internucleoside linkages, 9 or more modified internucleoside linkages, 10 or more modified internucleoside linkages, 11 or more modified internucleoside linkages, 12 or more modified internucleoside linkages, 13 or more modified internucleoside linkages, 14 or more modified internucleoside linkages, 15 or more modified internucleoside linkages, 16 or more modified internucleoside linkages, 17 or more modified internucleoside linkages, 18 or more modified internucleoside linkages, 19 or more modified internucleoside linkages, or 20 or more modified internucleoside linkages.

In some embodiments, the composition comprises an oligonucleotide that inhibits the expression of FGG, wherein the oligonucleotide comprises the modified nucleoside. In some embodiments, the modified nucleoside comprises a locked nucleic acid (LNA), hexitol nucleic acid (HLA), cyclohexene nucleic acid (CeNA), 2′-methoxyethyl, 2′-O-alkyl, 2-O-allyl, 2′-fluoro, or 2′-deoxy, or a combination thereof. In some embodiments, the modified nucleoside comprises a LNA. In some embodiments, the modified nucleoside comprises a 2′,4′ constrained ethyl nucleic acid. In some embodiments, the modified nucleoside comprises HLA. In some embodiments, the modified nucleoside comprises CeNA. In some embodiments, the modified nucleoside comprises a 2′-methoxyethyl group. In some embodiments, the modified nucleoside comprises a 2′-O-alkyl group. In some embodiments, the modified nucleoside comprises a 2′-O-allyl group. In some embodiments, the modified nucleoside comprises a 2′-fluoro group. In some embodiments, the modified nucleoside comprises a 2′-deoxy group. In some embodiments, the modified nucleoside comprises a 2′-O-methyl nucleoside, 2′-deoxyfluoro nucleoside, 2′-O—N-methylacetamido (2′-O-NMA) nucleoside, a 2′-O-dimethylaminoethoxyethyl (2′-O-DMAEOE) nucleoside, 2′-O-aminopropyl (2′-O-AP) nucleoside, or 2′-ara-F, or a combination thereof. In some embodiments, the modified nucleoside comprises a 2′-O-methyl nucleoside. In some embodiments, the modified nucleoside comprises a 2′-deoxyfluoro nucleoside. In some embodiments, the modified nucleoside comprises a 2′-O-NMA nucleoside. In some embodiments, the modified nucleoside comprises a 2′-O-DMAEOE nucleoside. In some embodiments, the modified nucleoside comprises a 2′-O-aminopropyl (2′-O-AP) nucleoside. In some embodiments, the modified nucleoside comprises 2′-ara-F. In some embodiments, the modified nucleoside comprises one or more 2′fluoro modified nucleosides. In some embodiments, the modified nucleoside comprises a 2′ O-alkyl modified nucleoside. Benefits of the modified nucleoside may include decreased toxicity or improved pharmacokinetics.

In some embodiments, the oligonucleotide comprises 1, 2, 3, 4, 5, 6, 7, 8, 9, 10, 11, 12, 13, 14, 15, 16, 17, 18, 19, 20, or 21 modified nucleosides, or a range of nucleosides defined by any two of the aforementioned numbers. In some embodiments, the oligonucleotide comprises no more than 19 modified nucleosides. In some embodiments, the oligonucleotide comprises no more than 21 modified nucleosides. In some embodiments, the oligonucleotide comprises 2 or more modified nucleosides, 3 or more modified nucleosides, 4 or more modified nucleosides, 5 or more modified nucleosides, 6 or more modified nucleosides, 7 or more modified nucleosides, 8 or more modified nucleosides, 9 or more modified nucleosides, 10 or more modified nucleosides, 11 or more modified nucleosides, 12 or more modified nucleosides, 13 or more modified nucleosides, 14 or more modified nucleosides, 15 or more modified nucleosides, 16 or more modified nucleosides, 17 or more modified nucleosides, 18 or more modified nucleosides, 19 or more modified nucleosides, 20 or more modified nucleosides, or 21 or more modified nucleosides.

In some embodiments, the composition comprises an oligonucleotide that inhibits the expression of FGG, wherein the oligonucleotide comprises a moiety attached at a 3′ or 5′ terminus of the oligonucleotide. Examples of moieties include a hydrophobic moiety or a sugar moiety, or a combination thereof. In some embodiments, the oligonucleotide is an siRNA having a sense strand, and the moiety is attached to a 5′ end of the sense strand. In some embodiments, the oligonucleotide is an siRNA having a sense strand, and the moiety is attached to a 3′ end of the sense strand. In some embodiments, the oligonucleotide is an siRNA having an antisense strand, and the moiety is attached to a 5′ end of the antisense strand. In some embodiments, the oligonucleotide is an siRNA having an antisense strand, and the moiety is attached to a 3′ end of the antisense strand. In some embodiments, the oligonucleotide is an ASO, and the moiety is attached to a 5′ end of the ASO. In some embodiments, the oligonucleotide is an ASO, and the moiety is attached to a 3′ end of the ASO.

In some embodiments, the composition comprises an oligonucleotide that inhibits the expression of FGG, wherein the oligonucleotide comprises a hydrophobic moiety. The hydrophobic moiety may be attached at a 3′ or 5′ terminus of the oligonucleotide. The hydrophobic moiety may include a lipid such as a fatty acid. The hydrophobic moiety may include a hydrocarbon. The hydrocarbon may be linear. The hydrocarbon may be non-linear. The hydrophobic moiety may include a lipid moiety or a cholesterol moiety, or a combination thereof.

In some embodiments, the composition comprises an oligonucleotide that inhibits the expression of FGG, wherein the oligonucleotide comprises a lipid attached at a 3′ or 5′ terminus of the oligonucleotide. In some embodiments, the lipid comprises cholesterol, myristoyl, palmitoyl, stearoyl, lithocholoyl, docosanoyl, docosahexaenoyl, myristyl, palmityl stearyl, or α-tocopherol, or a combination thereof.

In some embodiments, the composition comprises an oligonucleotide that inhibits the expression of FGG, wherein the oligonucleotide comprises a sugar moiety. The sugar moiety may include an N-acetyl galactose moiety (e.g. a N-acetylgalactosamine (GalNAc) moiety), an N-acetyl glucose moiety (e.g. an N-acetylglucosamine (GlcNAc) moiety), a fucose moiety, or a mannose moiety. The sugar moiety may include 1, 2, 3, or more sugar molecules. The sugar moiety may be attached at a 3′ or 5′ terminus of the oligonucleotide. The sugar moiety may include an N-acetyl galactose moiety. The sugar moiety may include an N-acetylgalactosamine (GalNAc) moiety. The sugar moiety may include an N-acetyl glucose moiety. The sugar moiety may include N-acetylglucosamine (GlcNAc) moiety. The sugar moiety may include a fucose moiety. The sugar moiety may include a mannose moiety. N-acetyl glucose, GlcNAc, fucose, or mannose may be useful for targeting macrophages since they may target or bind a mannose receptor such as CD206.

In some embodiments, the composition comprises an oligonucleotide that inhibits the expression of FGG, wherein the oligonucleotide comprises an N-acetylgalactosamine (GalNAc) moiety. GalNAc may be useful for hepatocyte targeting, neural (e.g., CNS (e.g., brain), or CSF targeting. The GalNAc moiety may include 1, 2, 3, or more GalNAc molecules. The GalNAc moiety may be attached at a 3′ or 5′ terminus of the oligonucleotide.

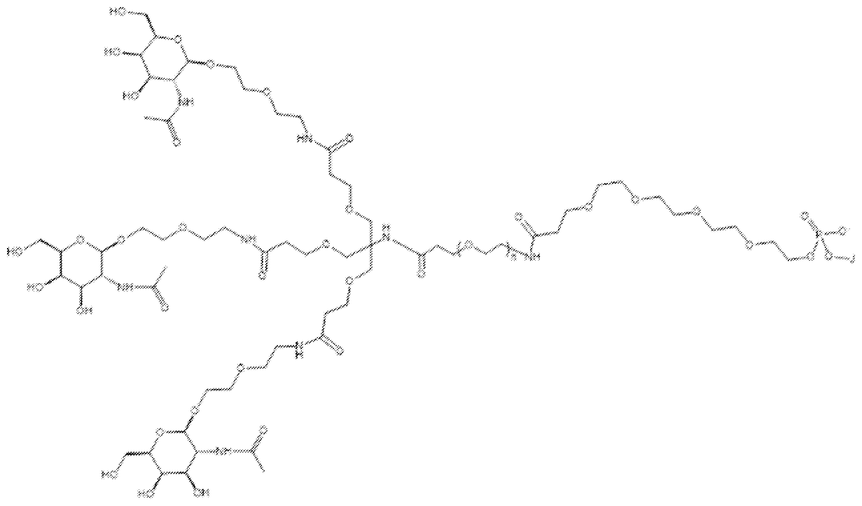

Non-limiting examples of GalNAc ligands are shown in FIG. 1 and FIG. 2. In some embodiments, the oligonucleotide is conjugated to the GalNAc ligand in FIG. 1. In the GalNAc ligand shown in FIG. 1, J indicates a point of attachment to an oligonucleotide. In some embodiments, J is at a 5′ end of the oligonucleotide. In some embodiments, J is at a 3′ end of the oligonucleotide. In the GalNAc ligand shown in FIG. 1, n may be any number. For example, n may be 1-10. In some embodiments, n is 1, 2, 3, 4, 5, 6, 7, 8, 9, or 10, or a range defined by any two of the aforementioned integers. In some embodiments, n is 2. In embodiments in which n is 2 and the oligonucleotide is connected at J, the GalNAc moiety may be referred to as “GalNAc #1” or “GalNAc1.”

In some embodiments, the oligonucleotide is conjugated to the GalNAc ligand in FIG. 2. The wavy line in FIG. 1 indicates a point of attachment to an oligonucleotide. In some embodiments, the wavy line is at a 5′ end of the oligonucleotide. In some embodiments, the wavy line is at a 3′ end of the oligonucleotide. In embodiments in which the oligonucleotide is connected at the wavy line, the GalNAc moiety may be referred to as “GalNAc #23” or “GalNAc23.”

The oligonucleotide may include purines. Examples of purines include adenine (A) or guanine (G), or modified versions thereof. The oligonucleotide may include pyrimidines. Examples of pyrimidines include cytosine (C), thymine (T), or uracil (U), or modified versions thereof.

In some embodiments, purines of the oligonucleotide comprise 2′ fluoro modified purines. In some embodiments, purines of the oligonucleotide comprise 2′-O-methyl modified purines. In some embodiments, purines of the oligonucleotide comprise a mixture of 2′ fluoro and 2′-O-methyl modified purines. In some embodiments, all purines of the oligonucleotide comprise 2′ fluoro modified purines. In some embodiments, all purines of the oligonucleotide comprise 2′-O-methyl modified purines. In some embodiments, all purines of the oligonucleotide comprise a mixture of 2′ fluoro and 2′-O-methyl modified purines. 2′-O-methyl may include 2′ O-methyl. Where 2′-O-methyl modifications are described, it is contemplated that a 2′-methyl modification may be included, and vice versa.

In some embodiments, pyrimidines of the oligonucleotide comprise 2′ fluoro modified pyrimidines. In some embodiments, pyrimidines of the oligonucleotide comprise 2′-O-methyl modified pyrimidines. In some embodiments, pyrimidines of the oligonucleotide comprise a mixture of 2′ fluoro and 2′-O-methyl modified pyrimidines. In some embodiments, all pyrimidines of the oligonucleotide comprise 2′ fluoro modified pyrimidines. In some embodiments, all pyrimidines of the oligonucleotide comprise 2′-O-methyl modified pyrimidines. In some embodiments, all pyrimidines of the oligonucleotide comprise a mixture of 2′ fluoro and 2′-O-methyl modified pyrimidines.

In some embodiments, purines of the oligonucleotide comprise 2′ fluoro modified purines, and pyrimidines of the oligonucleotide comprise a mixture of 2′ fluoro and 2′-O-methyl modified pyrimidines. In some embodiments, purines of the oligonucleotide comprise 2′-O-methyl modified purines, and pyrimidines of the oligonucleotide comprise a mixture of 2′ fluoro and 2′-O-methyl modified pyrimidines. In some embodiments, purines of the oligonucleotide comprise 2′ fluoro modified purines, and pyrimidines of the oligonucleotide comprise 2′-O-methyl modified pyrimidines. In some embodiments, purines of the oligonucleotide comprise 2′-O-methyl modified purines, and pyrimidines of the oligonucleotide comprise 2′ fluoro modified pyrimidines. In some embodiments, pyrimidines of the oligonucleotide comprise 2′ fluoro modified pyrimidines, and purines of the oligonucleotide comprise a mixture of 2′ fluoro and 2′-O-methyl modified purines. In some embodiments, pyrimidines of the oligonucleotide comprise 2′-O-methyl modified pyrimidines, and purines of the oligonucleotide comprise a mixture of 2′ fluoro and 2′-O-methyl modified purines. In some embodiments, pyrimidines of the oligonucleotide comprise 2′ fluoro modified pyrimidines, and purines of the oligonucleotide comprise 2′-O-methyl modified purines. In some embodiments, pyrimidines of the oligonucleotide comprise 2′-O-methyl modified pyrimidines, and purines of the oligonucleotide comprise 2′ fluoro modified purines.

In some embodiments, all purines of the oligonucleotide comprise 2′ fluoro modified purines, and all pyrimidines of the oligonucleotide comprise a mixture of 2′ fluoro and 2′-O-methyl modified pyrimidines. In some embodiments, all purines of the oligonucleotide comprise 2′-O-methyl modified purines, and all pyrimidines of the oligonucleotide comprise a mixture of 2′ fluoro and 2′-O-methyl modified pyrimidines. In some embodiments, all purines of the oligonucleotide comprise 2′ fluoro modified purines, and all pyrimidines of the oligonucleotide comprise 2′-O-methyl modified pyrimidines. In some embodiments, all purines of the oligonucleotide comprise 2′-O-methyl modified purines, and all pyrimidines of the oligonucleotide comprise 2′ fluoro modified pyrimidines. In some embodiments, all pyrimidines of the oligonucleotide comprise 2′ fluoro modified pyrimidines, and all purines of the oligonucleotide comprise a mixture of 2′ fluoro and 2′-O-methyl modified purines. In some embodiments, all pyrimidines of the oligonucleotide comprise 2′-O-methyl modified pyrimidines, and all purines of the oligonucleotide comprise a mixture of 2′ fluoro and 2′-O-methyl modified purines. In some embodiments, all pyrimidines of the oligonucleotide comprise 2′ fluoro modified pyrimidines, and all purines of the oligonucleotide comprise 2′-O-methyl modified purines. In some embodiments, all pyrimidines of the oligonucleotide comprise 2′-O-methyl modified pyrimidines, and all purines of the oligonucleotide comprise 2′ fluoro modified purines.

In some cases, the oligonucleotide comprises a particular modification pattern. In some embodiments, position 9 counting from the 5′ end of the of a strand of the oligonucleotide may have a 2′F modification. In some embodiments, when position 9 of a strand of the oligonucleotide is a pyrimidine, then all purines in a strand of the oligonucleotide have a 2′OMe modification. In some embodiments, when position 9 is the only pyrimidine between positions 5 and 11 of the sense stand, then position 9 is the only position with a 2′F modification in a strand of the oligonucleotide. In some embodiments, when position 9 and only one other base between positions 5 and 11 of a strand of the oligonucleotide are pyrimidines, then both of these pyrimidines are the only two positions with a 2′F modification in a strand of the oligonucleotide. In some embodiments, when position 9 and only two other bases between positions 5 and 11 of a strand of the oligonucleotide are pyrimidines, and those two other pyrimidines are in adjacent positions so that there would be not three 2′F modifications in a row, then any combination of 2′F modifications can be made that give three 2′F modifications in total. In some embodiments, when there are more than 2 pyrimidines between positions 5 and 11 of a strand of the oligonucleotide, then all combinations of pyrimidines having the 2′F modification are allowed that have three to five 2′F modifications in total, provided that a strand of the oligonucleotide does not have three 2′F modifications in a row. In some cases, a strand of the oligonucleotide of any of the siRNAs comprises a modification pattern which conforms to any or all of these a strand of the oligonucleotide rules.

In some embodiments, when position 9 of a strand of the oligonucleotide is a purine, then all purines in a strand of the oligonucleotide have a 2′OMe modification. In some embodiments, when position 9 is the only purine between positions 5 and 11 of the sense stand, then position 9 is the only position with a 2′F modification in a strand of the oligonucleotide. In some embodiments, when position 9 and only one other base between positions 5 and 11 of a strand of the oligonucleotide are purines, then both of these purines are the only two positions with a 2′F modification in a strand of the oligonucleotide. In some embodiments, when position 9 and only two other bases between positions 5 and 11 of a strand of the oligonucleotide are purines, and those two other purines are in adjacent positions so that there would be not three 2′F modifications in a row, then any combination of 2′F modifications can be made that give three 2′F modifications in total. In some embodiments, when there are more than 2 purines between positions 5 and 11 of a strand of the oligonucleotide, then all combinations of purines having the 2′F modification are allowed that have three to five 2′F modifications in total, provided that a strand of the oligonucleotide does not have three 2′F modifications in a row. In some cases, a strand of the oligonucleotide of any of the siRNAs comprises a modification pattern which conforms to any or all of these a strand of the oligonucleotide rules.

In some cases, position 9 of a strand of the oligonucleotide can be a 2′deoxy. In these cases, 2′F and 2′OMe modifications may occur at the other positions of a strand of the oligonucleotide. In some cases, a strand of the oligonucleotide of any of the siRNAs comprises a modification pattern which conforms to these a strand of the oligonucleotide rules.

In some embodiments, position nine of the sense strand comprises a 2′ fluoro-modified pyrimidine. In some embodiments, all purines of the sense strand comprise 2′-O-methyl modified purines. In some embodiments, 1, 2, 3, 4, or 5 pyrimidines between positions 5 and 11 comprise a 2′flouro-modified pyrimidine, provided there are not three 2′ fluoro-modified pyrimidines in a row. In some embodiments, the odd-numbered positions of the antisense strand comprise 2′-O-methyl modified nucleotides. In some embodiments, the even-numbered positions of the antisense strand comprise 2′flouro-modified nucleotides and unmodified deoxyribonucleotide. In some embodiments, the even-numbered positions of the antisense strand comprise 2′flouro-modified nucleotides, 2′-O-methyl modified nucleotides and unmodified deoxyribonucleotide. In some embodiments, position nine of the sense strand comprises a 2′ fluoro-modified pyrimidine; all purines of the sense strand comprises 2′-O-methyl modified purines; 1, 2, 3, 4, or 5 pyrimidines between positions 5 and 11 comprise a 2′flouro-modified pyrimidine, provided there are not three 2′ fluoro-modified pyrimidines in a row; the odd-numbered positions of the antisense strand comprise 2′-O-methyl modified nucleotides; and the even-numbered positions of the antisense strand comprise 2′flouro-modified nucleotides and unmodified deoxyribonucleotides.

In some embodiments, position nine of the sense strand comprises a 2′ fluoro-modified purine. In some embodiments, all pyrimidines of the sense strand comprise 2′-O-methyl modified purines. In some embodiments, 1, 2, 3, 4, or 5 purines between positions 5 and 11 comprise a 2′flouro-modified purine, provided there are not three 2′ fluoro-modified purine in a row. In some embodiments, the odd-numbered positions of the antisense strand comprise 2′-O-methyl modified nucleotides. In some embodiments, the even-numbered positions of the antisense strand comprise 2′flouro-modified nucleotides and unmodified deoxyribonucleotide. In some embodiments, the even-numbered positions of the antisense strand comprise 2′flouro-modified nucleotides, 2′-O-methyl modified nucleotides and unmodified deoxyribonucleotide. In some embodiments, position nine of the sense strand comprises a 2′ fluoro-modified purine; all pyrimidine of the sense strand comprises 2′-O-methyl modified pyrimidines; 1, 2, 3, 4, or 5 purines between positions 5 and 11 comprise a 2′flouro-modified purines, provided there are not three 2′ fluoro-modified purines in a row; the odd-numbered positions of the antisense strand comprise 2′-O-methyl modified nucleotides; and the even-numbered positions of the antisense strand comprise 2′flouro-modified nucleotides and unmodified deoxyribonucleotides. In some embodiments, there are not three 2′ fluoro-modified purines in a row. In some embodiments, there are not three 2′ fluoro-modified pyrimidines in a row.

In some embodiments, position nine of the sense strand comprises an unmodified deoxyribonucleotide. In some embodiments, positions 5, 7, and 8 of the sense strand comprise 2′fluoro-modifed nucleotides. In some embodiments, all pyrimidines in positions 10 to 21 of the sense strand comprise 2′-O-methyl modified pyrimidines and all purines in positions 10 to 21 of the comprise 2′-O-methyl modified purines or 2′fluoro-modified purines. In some embodiments, the odd-numbered positions of the antisense strand comprise 2′-O-methyl modified nucleotides. In some embodiments, the even-numbered positions of the antisense strand comprise 2′flouro-modified nucleotides and unmodified deoxyribonucleotides. In some embodiments, the even-numbered positions of the antisense strand comprise 2′flouro-modified nucleotides, 2′-O-methyl modified nucleotides and unmodified deoxyribonucleotides. In some embodiments, position nine of the sense strand comprises an unmodified deoxyribonucleotide; positions 5, 7, and 8 of the sense strand comprise 2′fluoro-modifed nucleotides; all pyrimidines in positions 10 to 21 of the sense strand comprise 2′-O-methyl modified pyrimidines and all purines in positions 10 to 21 of the comprise 2′-O-methyl modified purines or 2′fluoro-modified purines; the odd-numbered positions of the antisense strand comprise 2′-O-methyl modified nucleotides; and the even-numbered positions of the antisense strand comprise 2′flouro-modified nucleotides and unmodified deoxyribonucleotides.

In some embodiments, position nine of the sense strand comprises an unmodified deoxyribonucleotide. In some embodiments, positions 5, 7, and 8 of the sense strand comprise 2′fluoro-modified nucleotides. In some embodiments, all purines in positions 10 to 21 of the sense strand comprise 2′-O-methyl modified purines and all pyrimidines in positions 10 to 21 of the comprise 2′-O-methyl modified pyrimidines or 2′fluoro-modified pyrimidines. In some embodiments, the odd-numbered positions of the antisense strand comprise 2′-O-methyl modified nucleotides. In some embodiments, the even-numbered positions of the antisense strand comprise 2′flouro-modified nucleotides and unmodified deoxyribonucleotides. In some embodiments, the even-numbered positions of the antisense strand comprise 2′flouro-modified nucleotides, 2′-O-methyl modified nucleotides and unmodified deoxyribonucleotides. In some embodiments, position nine of the sense strand comprises an unmodified deoxyribonucleotide; positions 5, 7, and 8 of the sense strand comprise 2′fluoro-modified nucleotides; all purines in positions 10 to 21 of the sense strand comprise 2′-O-methyl modified purines and all pyrimidines in positions 10 to 21 of the comprise 2′-O-methyl modified pyrimidines or 2′fluoro-modified pyrimidines; the odd-numbered positions of the antisense strand comprise 2′-O-methyl modified nucleotides; and the even-numbered positions of the antisense strand comprise 2′flouro-modified nucleotides and unmodified deoxyribonucleotide.

In some embodiments, the moiety includes a negatively charged group attached at a 5′ end of the oligonucleotide. This may be referred to as a 5′-end group. In some embodiments, the negatively charged group is attached at a 5′ end of an antisense strand of an siRNA disclosed herein. The 5′-end group may be or include a 5′-end phosphorothioate, 5′-end phosphorodithioate, 5′-end vinylphosphonate (5′-VP), 5′-end methylphosphonate, 5′-end cyclopropyl phosphonate, or a 5′-deoxy-5′-C-malonyl. The 5′-end group may comprise 5′-VP. In some embodiments, the 5′-VP comprises a trans-vinylphosphate or cis-vinylphosphate. The 5′-end group may include an extra 5′ phosphate. A combination of 5′-end groups may be used.

In some embodiments, the oligonucleotide includes a negatively charged group. The negatively charged group may aid in cell or tissue penetration. The negatively charged group may be attached at a 5′ or 3′ end (e.g. a 5′ end) of the oligonucleotide. This may be referred to as an end group. The end group may be or include a phosphorothioate, phosphorodithioate, vinylphosphonate, methylphosphonate, cyclopropyl phosphonate, or a deoxy-C-malonyl. The end group may include an extra 5′ phosphate such as an extra 5′ phosphate. A combination of end groups may be used.

In some embodiments, the oligonucleotide includes a phosphate mimic. In some embodiments, the phosphate mimic comprises vinyl phosphonate. In some embodiments, the vinyl phosphonate comprises a trans-vinylphosphate. In some embodiments, the vinyl phosphonate comprises a cis-vinylphosphate. An example of a nucleotide that includes a vinyl phosphonate is shown below.

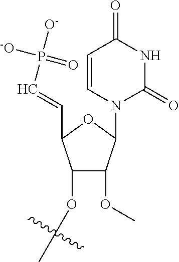

5′ vinylphosphonate 2′ O Methyl Uridine

In some embodiments, the vinyl phosphonate increases the stability of the oligonucleotide. In some embodiments, the vinyl phosphonate increases the accumulation of the oligonucleotide in tissues. In some embodiments, the vinyl phosphonate protects the oligonucleotide from an exonuclease or a phosphatase. In some embodiments, the vinyl phosphonate improves the binding affinity of the oligonucleotide with the siRNA processing machinery.

In some embodiments, the oligonucleotide includes 1 vinyl phosphonate. In some embodiments, the oligonucleotide includes 2 vinyl phosphonates. In some embodiments, the oligonucleotide includes 3 vinyl phosphonates. In some embodiments, the oligonucleotide includes 4 vinyl phosphonates. In some embodiments, the antisense strand of the oligonucleotide comprises a vinyl phosphonate at the 5′ end. In some embodiments, the antisense strand of the oligonucleotide comprises a vinyl phosphonate at the 3′ end. In some embodiments, the sense strand of the oligonucleotide comprises a vinyl phosphonate at the 5′ end. In some embodiments, the sense strand of the oligonucleotide comprises a vinyl phosphonate at the 3′ end.

### 1. Hydrophobic Moieties

In some embodiments, the composition comprises an oligonucleotide that inhibits the expression of FGG, wherein the oligonucleotide comprises a hydrophobic moiety. The hydrophobic moiety may be attached at a 3′ or 5′ terminus of the oligonucleotide. The hydrophobic moiety may include a lipid such as a fatty acid. The hydrophobic moiety may include a hydrocarbon. The hydrocarbon may be linear. The hydrocarbon may be non-linear. The hydrophobic moiety may include a lipid moiety or a cholesterol moiety, or a combination thereof.

In some embodiments, the composition comprises an oligonucleotide that inhibits the expression of FGG, wherein the oligonucleotide comprises a lipid attached at a 3′ or 5′ terminus of the oligonucleotide. In some embodiments, the lipid comprises cholesterol, myristoyl, palmitoyl, stearoyl, lithocholoyl, docosanoyl, docosahexaenoyl, myristyl, palmityl, stearyl, or α-tocopherol, or a combination thereof.

In some embodiments, the composition comprises an oligonucleotide that inhibits the expression of FGG, wherein the oligonucleotide comprises a hydrophobic ligand or moiety. In some embodiments, the hydrophobic ligand or moiety comprises cholesterol. In some embodiments, the hydrophobic ligand or moiety comprises a cholesterol derivative. In some embodiments, the hydrophobic ligand or moiety is attached at a 3′ terminus of the oligonucleotide. In some embodiments, the hydrophobic ligand or moiety s attached at a 5′ terminus of the oligonucleotide. In some embodiments, the composition comprises a sense strand, and the hydrophobic ligand or moiety is attached to the sense strand (e.g. attached to a 5′ end of the sense strand, or attached to a 3′ end of the sense strand). In some embodiments, the composition comprises an antisense strand, and the hydrophobic ligand or moiety is attached to the antisense strand (e.g. attached to a 5′ end of the antisense strand, or attached to a 3′ end of the antisense strand). In some embodiments, the composition comprises a hydrophobic ligand or moiety attached at a 3′ or 5′ terminus of the oligonucleotide.

In some embodiments, a hydrophobic moiety is attached to the oligonucleotide (e.g. a sense strand and/or an antisense strand of a siRNA). In some embodiments, a hydrophobic moiety is attached at a 3′ terminus of the oligonucleotide. In some embodiments, a hydrophobic moiety is attached at a 5′ terminus of the oligonucleotide. In some embodiments, the hydrophobic moiety comprises cholesterol. In some embodiments, the hydrophobic moiety includes a cyclohexanyl.

In some embodiments, the composition comprises an oligonucleotide that inhibits the expression of FGG, wherein the oligonucleotide comprises a lipid attached at a 3′ or 5′ terminus of the oligonucleotide. In some embodiments, a lipid is attached at a 3′ terminus of the oligonucleotide. In some embodiments, a lipid is attached at a 5′ terminus of the oligonucleotide. In some embodiments, the lipid comprises cholesterol, myristoyl, palmitoyl, stearoyl, lithocholoyl, docosanoyl, docosahexaenoyl, myristyl, palmityl, stearyl, or α-tocopherol, or a combination thereof. In some embodiments, the lipid comprises stearyl, lithocholyl, docosanyl, docosahexaenyl, or myristyl. In some embodiments, the lipid comprises cholesterol. In some embodiments, the lipid includes a sterol such as cholesterol. In some embodiments, the lipid comprises stearyl, t-butylphenol, n-butylphenol, octylphenol, dodecylphenol, phenyl n-dodecyl, octadecylbenzamide, hexadecylbenzamide, or octadecylcyclohexyl. In some embodiments, the lipid comprises phenyl para C12.

In some embodiments, the oligonucleotide comprises any aspect of the following structure:

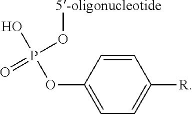

In some embodiments, the oligonucleotide comprises any aspect of the following structure:

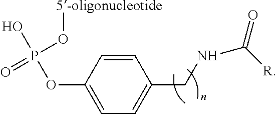

In some embodiments, the oligonucleotide comprises any aspect of the following structure:

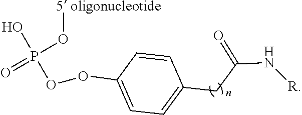

In some embodiments, the oligonucleotide comprises any aspect of the following structure: The aspect included in the oligonucleotide may include the entire structure, or may include the lipid moiety, of any of the structures shown. In some embodiments, n is 1-3. In some embodiments, n is 1. In some embodiments, n is 2. In some embodiments, n is 3. In some embodiments, R is an alkyl group. In some embodiments, the alkyl group contains 1, 2, 3, 4, 5, 6, 7, 8, 9, 10, 11, 12, 13, 14, 15, 16, 17, 18, 19, or 20 carbons. In some embodiments, the alkyl group contains 4, 5, 6, 7, 8, 9, 10, 11, 12, 13, 14, 15, 16, 17, or 18 carbons, or a range defined by any two of the aforementioned numbers of carbons. In some embodiments, the alkyl group contains 4-18 carbons. In some embodiments, the lipid moiety comprises an alcohol or ether.

In some embodiments, the lipid includes a fatty acid. In some embodiments, the lipid comprises a lipid depicted in Table 1. The example lipid moieties in Table 1 are shown attached at a 5′ end of an oligonucleotide, in which the 5′ terminal phosphate of the oligonucleotide is shown with the lipid moiety. In some embodiments, a lipid moiety in Table 1 may be attached at a different point of attachment than shown. For example, the point of attachment of any of the lipid moieties in the table may be at a 3′ oligonucleotide end. In some embodiments, the lipid is used for targeting the oligonucleotide to a non-hepatic cell or tissue.

TABLE 1
Hydrophobic moiety examples

| Hydrophobic | | |
|---|---|---|
| Moiety | Hydrophobic | |
| Description | Moiety Name | Example Conjugation |
| stearyl | ETL3 | 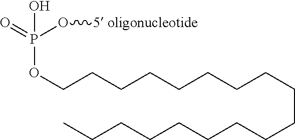 |
| t-butylphenyl | ETL7 | 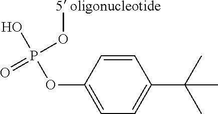 |
| n-butylphenyl | ETL8 | 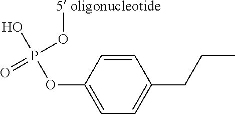 |
| octylphenyl | ETL9 | 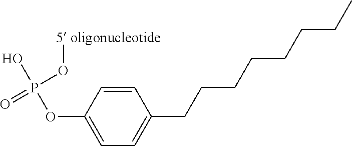 |
| dodecylphenyl | ETL10 | 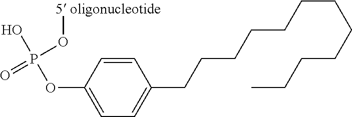 |
| phenyl n- dodecyl | ETL12 | 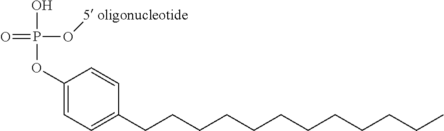 |
| octadecyl- benzamide | ETL13 | 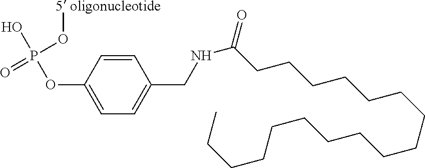 |
| hexadecyl- benzamide | ETL15 | 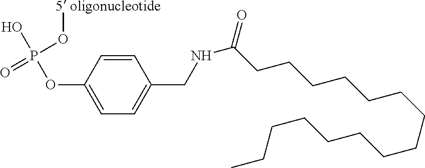 |
| octadecyl- cyclohexyl | ETL16 | 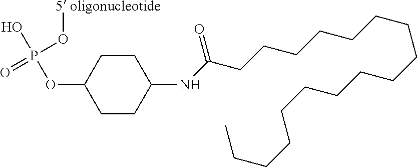 |
| myristamido methylphenyl | ETL18 | 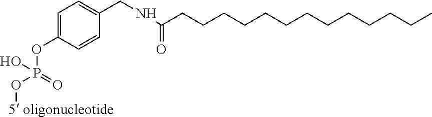 |
| lauramido methylphenyl | ETL 19 | 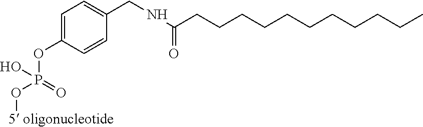 |
| phenethyl- palmityl | ETL20 | 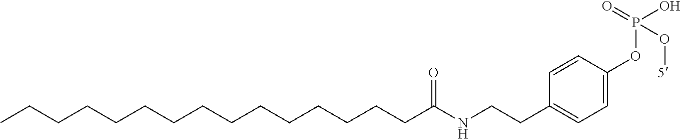 |

In some embodiments, the lipid or lipid moiety includes 16 to 18 carbons. In some embodiments, the lipid includes 16 carbons. In some embodiments, the lipid includes 17 carbons. In some embodiments, the lipid includes 18 carbons. In some embodiments, the lipid moiety includes 16 carbons. In some embodiments, the lipid moiety includes 17 carbons. In some embodiments, the lipid moiety includes 18 carbons.

The hydrophobic moiety may include a linker that comprises a carbocycle. The carbocycle may be six-membered. Some examples of a carbocycle include phenyl or cyclohexyl. The linker may include a phenyl. The linker may include a cyclohexyl. The lipid may be attached to the carbocycle, which may in turn be attached at a phosphate (e.g. 5′ or 3′ phosphate) of the oligonucleotide. In some embodiments, the lipid or hydrocarbon, and the end of the sense are connected to the phenyl or cyclohexyl linker in the 1,4; 1,3; or 1,2 substitution pattern (e.g. the para, meta, or ortho phenyl configuration). In some embodiments, the lipid or hydrocarbon, and the end of the sense are connected to the phenyl or cyclohexyl linker in the 1,4 substitution pattern (e.g. the para phenyl configuration). The lipid may be attached to the carbocycle in the 1,4 substitution pattern relative to the oligonucleotide. The lipid may be attached to the carbocycle in the 1,3 substitution pattern relative to the oligonucleotide. The lipid may be attached to the carbocycle in the 1,2 substitution pattern relative to the oligonucleotide. The lipid may be attached to the carbocycle in the ortho orientation relative to the oligonucleotide. The lipid may be attached to the carbocycle in the para orientation relative to the oligonucleotide. The lipid may be attached to the carbocycle in the meta orientation relative to the oligonucleotide.

The lipid moiety may comprise or consist of the following structure

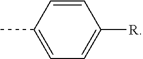

In some embodiments, the lipid moiety comprises or consists of the following structure:

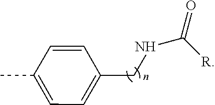

In some embodiments, the lipid moiety comprises the following structure:

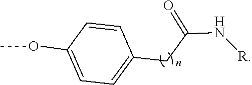

In some embodiments, the lipid moiety comprises or consist of the following structure:

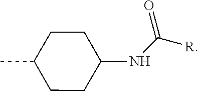

In some embodiments, the dotted line indicates a covalent connection. The covalent connection may between an end of the sense or antisense strand. For example, the connection may be to the 5′ end of the sense strand. In some embodiments, n is 0-3. In some embodiments, n is 1-3. In some embodiments, n is 0. In some embodiments, n is 1. In some embodiments, n is 2. In some embodiments, n is 3. In some embodiments, n is 4. In some embodiments, n is 5. In some embodiments, n is 1, 2, 3, 4, 5, 6, 7, 8, 9, or 10. In some embodiments, R is an alkyl group. In some embodiments, the alkyl group contains 1, 2, 3, 4, 5, 6, 7, 8, 9, 10, 11, 12, 13, 14, 15, 16, 17, 18, 19, or 20 carbons. In some embodiments, the alkyl group contains 4, 5, 6, 7, 8, 9, 10, 11, 12, 13, 14, 15, 16, 17, or 18 carbons, or a range defined by any two of the aforementioned numbers of carbons. In some embodiments, R comprises or consists of an alkyl group containing 4-18 carbons.

The lipid moiety may be attached at a 5′ end of the oligonucleotide. The 5′ end may have one phosphate linking the lipid moiety to a 5′ carbon of a sugar of the oligonucleotide. The 5′ end may have two phosphates linking the lipid moiety to a 5′ carbon of a sugar of the oligonucleotide. The 5′ end may have three phosphates linking the lipid moiety to a 5′ carbon of a sugar of the oligonucleotide. The 5′ end may have one phosphate connected to the 5′ carbon of a sugar of the oligonucleotide, where the one phosphate is connected to the lipid moiety. The 5′ end may have two phosphates connected to the 5′ carbon of a sugar of the oligonucleotide, where the one of the two phosphates is connected to the lipid moiety. The 5′ end may have three phosphates connected to the 5′ carbon of a sugar of the oligonucleotide, where the one of the three phosphates is connected to the lipid moiety. The sugar may include a ribose. The sugar may include a deoxyribose. The sugar may be modified a such as a 2′ modified sugar (e.g. a 2′ O-methyl or 2′ fluoro ribose). A phosphate of the 5′ end may include a modification such as a sulfur in place of an oxygen. Two phosphates of the 5′ end may include a modification such as a sulfur in place of an oxygen. Three phosphates of the 5′ end may include a modification such as a sulfur in place of an oxygen.

In some embodiments, the oligonucleotide includes 1 lipid moiety. In some embodiments, the oligonucleotide includes 2 lipid moieties. In some embodiments, the oligonucleotide includes 3 lipid moieties. In some embodiments, the oligonucleotide includes 4 lipid moieties.

Some embodiments relate to a method of making an oligonucleotide comprising a hydrophobic conjugate. A strategy for making hydrophobic conjugates may include use of a phosphoramidite reagent based upon a 6-membered ring alcohol such as a phenol or cyclohexanol. The phosphoramidite may be reacted to a nucleotide to connect the nucleotide to the hydrophobic moiety, and thereby produce the hydrophobic conjugate. Some examples of phosphoramidite reagents that may be used to produce a hydrophobic conjugate are provided as follows:

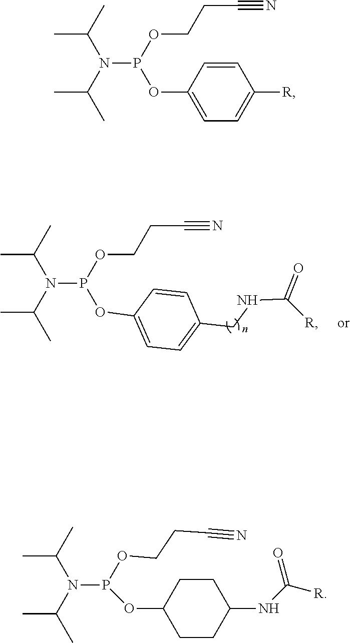

In some embodiments, n is 1-3. In some embodiments, n is 1. In some embodiments, n is 2. In some embodiments, n is 3. In some embodiments, R is an alkyl group. In some embodiments, the alkyl group contains 1, 2, 3, 4, 5, 6, 7, 8, 9, 10, 11, 12, 13, 14, 15, 16, 17, 18, 19, or 20 carbons. In some embodiments, the alkyl group contains 4, 5, 6, 7, 8, 9, 10, 11, 12, 13, 14, 15, 16, 17, or 18 carbons, or a range defined by any two of the aforementioned numbers of carbons. In some embodiments, R comprises or consists of an alkyl group containing 4-18 carbons. Any one of the phosphoramidite reagents may be reacted to a 5′ end of an oligonucleotide to produce an oligonucleotide comprising a hydrophobic moiety. In some embodiments, the phosphoramidite reagents is reacted to a 5′ end of a sense strand of an siRNA. The sense strand may then be hybridized to an antisense strand to form a duplex. The hybridization may be performed by incubating the sense and antisense strands in solution at a given temperature. The temperature may be gradually reduced. The temperature may comprise or include a temperature comprising an annealing temperature for the sense and antisense strands. The temperature may be below or include a temperature below the annealing temperature for the sense and antisense strands. The temperature may be below a melting temperature of the sense and antisense strands.

The lipid may be attached to the oligonucleotide by a linker. The linker may include a polyethyleneglycol (e.g. tetraethyleneglycol).

The modifications described herein may be useful for delivery to a cell or tissue, for example, extrahepatic delivery or targeting of an oligonucleotide composition. The modifications described herein may be useful for targeting an oligonucleotide composition to a cell or tissue.

### 2. Sugar Moieties

In some embodiments, the composition comprises an oligonucleotide that inhibits the expression of FGG, wherein the oligonucleotide comprises a sugar moiety. The sugar moiety may include an N-acetyl galactose moiety (e.g. an N-acetylgalactosamine (GalNAc) moiety), an N-acetyl glucose moiety (e.g. an N-acetylglucosaminc (GlcNAc) moiety), a fucose moiety, or a mannose moiety. The sugar moiety may include 1, 2, 3, or more sugar molecules. The sugar moiety may be attached at a 3′ or 5′ terminus of the oligonucleotide. The sugar moiety may include an N-acetyl galactose moiety. The sugar moiety may include an N-acetylgalactosamine (GalNAc) moiety. The sugar moiety may include an N-acetyl glucose moiety. The sugar moiety may include N-acetylglucosamine (GlcNAc) moiety. The sugar moiety may include a fucose moiety. The sugar moiety may include a mannose moiety. N-acetyl glucose, GlcNAc, fucose, or mannose may be useful for targeting macrophages when they target or bind a mannose receptor such as CD206. The sugar moiety may be useful for binding or targeting an asialoglycoprotein receptor such as an asialoglycoprotein receptor of a hepatocyte. The GalNAc moiety may bind to an asialoglycoprotein receptor. The GalNAc moiety may target a hepatocyte.

In some embodiments, the composition comprises an oligonucleotide that inhibits the expression of FGG, wherein the oligonucleotide comprises an N-acetylgalactosamine (GalNAc) moiety. GalNAc may be useful for hepatocyte targeting. The GalNAc moiety may include a bivalent or trivalent branched linker. The oligo may be attached to 1, 2 or 3 GalNAcs through a bivalent or trivalent branched linker. The GalNAc moiety may include 1, 2, 3, or more GalNAc molecules. The GalNAc moiety may be attached at a 3′ or 5′ terminus of the oligonucleotide.

In some embodiments, the composition comprises an oligonucleotide that inhibits the expression of FGG, wherein the oligonucleotide comprises an N-acetylgalactosamine (GalNAc) ligand for hepatocyte targeting. In some embodiments, the composition comprises GalNAc. In some embodiments, the composition comprises a GalNAc derivative. In some embodiments, the GalNAc ligand is attached at a 3′ terminus of the oligonucleotide. In some embodiments, the GalNAc ligand is attached at a 5′ terminus of the oligonucleotide. In some embodiments, the composition comprises a sense strand, and the GalNAc ligand is attached to the sense strand (e.g. attached to a 5′ end of the sense strand, or attached to a 3′ end of the sense strand). In some embodiments, the composition comprises an antisense strand, and the GalNAc ligand is attached to the antisense strand (e.g. attached to a 5′ end of the antisense strand, or attached to a 3′ end of the antisense strand). In some embodiments, the composition comprises a GalNAc ligand attached at a 3′ or 5′ terminus of the oligonucleotide.

Disclosed herein, in some embodiments, are compositions comprising an oligonucleotide that inhibits the expression of FGG, wherein the oligonucleotide comprises a GalNAc moiety. The GalNAc moiety may be included in any formula, structure, or GalNAc moiety shown below. In some embodiments, described herein is a compound (e.g. oligonucleotide) represented by Formula (I) or (II):

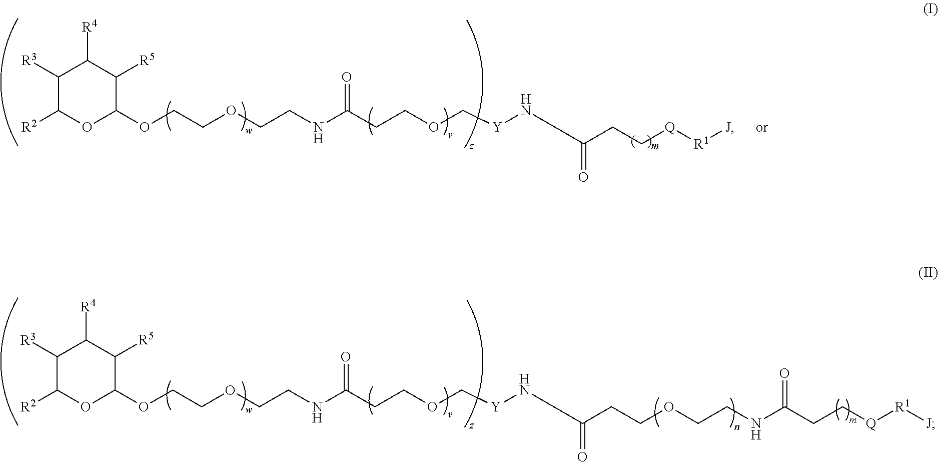

or a salt thereof, wherein
J is an oligonucleotide;
each w is independently selected from any value from 1 to 20;
each v is independently selected from any value from 1 to 20;
n is selected from any value from 1 to 20;
m is selected from any value from 1 to 20;
z is selected from any value from 1 to 3, wherein
   if z is 3, Y is C
   if z is 2, Y is CR⁶, or
   if z is 1, Y is C(R⁶)₂;
Q is selected from:
   C₃₋₁₀ carbocycle optionally substituted with one or more substituents independently selected from halogen, —CN, —NO₂, —OR⁷, —SR⁷, —N(R⁷)₂, —C(O)R⁷, —C(O)N(R⁷)₂, —N(R⁷)C(O)R⁷, —N(R⁷)C(O)N(R⁷)₂, —OC(O)N(R⁷)₂, —N(R⁷)C(O)OR⁷, —C(O)OR⁷, —OC(O)R⁷, —S(O)R⁷, and C₁₋₆ alkyl, wherein the C₁₋₆ alkyl, is optionally substituted with one or more substituents independently selected from halogen, —CN, —OH, —SH, —NO₂, and —NH₂;
R¹ is a linker selected from:
   —O—, —S—, —N(R⁷)—, —C(O)—, —C(O)N(R⁷)—, —N(R⁷)C(O)—, —N(R⁷)C(O)N(R⁷)—, —OC(O)N(R⁷)—, —N(R⁷)C(O)O—, —C(O)O—, —OC(O)—, —S(O)—, —S(O)₂—, —OS(O)₂—, —OP(O)(OR⁷)O—, —SP(O)(OR⁷)O—, —OP(S)(OR⁷)O—, —OP(O)(SR⁷)O—, —OP(O)(OR⁷)S—, —OP(O)(O⁻)O—, —SP(O)(O⁻)O—, —OP(S)(O⁻)O—, —OP(O)(S⁻)O—, —OP(O)(O⁻)S—, —OP(O)(OR⁷)NR⁷—, —OP(O)(N(R⁷)₂)NR⁷—, —OP(OR⁷)O—, —OP(N(R⁷)₂)O—, —OP(OR⁷)N(R⁷)—, and —OPN(R⁷)₂NR⁷—;
each R² is independently selected from:
   C₁₋₆ alkyl optionally substituted with one or more substituents independently selected from halogen, —OR⁷, —SR⁷, —N(R⁷)₂, —C(O)R⁷, —C(O)N(R⁷)₂, —N(R⁷)C(O)R⁷, —N(R⁷)C(O)N(R⁷)₂, —OC(O)N(R⁷)₂, —N(R⁷)C(O)OR⁷, —C(O)OR⁷, —OC(O)R⁷, and —S(O)R⁷;
R³ and R⁴ are each independently selected from:
   —OR⁷, —SR⁷, —N(R⁷)₂, —C(O)R⁷, —C(O)N(R⁷)₂, —N(R⁷)C(O)R⁷, —N(R⁷)C(O)N(R⁷)₂, —OC(O)N(R⁷)₂, —N(R⁷)C(O)OR⁷, —C(O)OR⁷, —OC(O)R⁷, and —S(O)R⁷;
each R⁵ is independently selected from:
   —OC(O)R⁷, —OC(O)N(R⁷)₂, —N(R⁷)C(O)R⁷, —N(R⁷)C(O)N(R⁷)₂, —N(R⁷)C(O)OR⁷, —C(O)R⁷, —C(O)OR⁷, and —C(O)N(R⁷)₂;
each R⁶ is independently selected from:
   hydrogen;
   halogen, —CN, —NO₂, —OR⁷, —SR⁷, —N(R⁷)₂, —C(O)R⁷, —C(O)N(R⁷)₂, —N(R⁷)C(O)R⁷, —N(R⁷)C(O)N(R⁷)₂, —OC(O)N(R⁷)₂, —N(R⁷)C(O)OR⁷, —C(O)OR⁷, —OC(O)R⁷, and —S(O)R⁷; and
   C₁₋₆ alkyl optionally substituted with one or more substituents independently selected from halogen, —CN, —NO₂, —OR⁷, —SR⁷, —N(R⁷)₂, —C(O)R⁷, —C(O)N(R⁷)₂, —N(R⁷)C(O)R⁷, —N(R⁷)C(O)N(R⁷)₂, —OC(O)N(R⁷)₂, —N(R⁷)C(O)OR⁷, —C(O)OR⁷, —OC(O)R⁷, and —S(O)R⁷;
each R⁷ is independently selected from:
   hydrogen;
   C₁₋₆ alkyl, C₂₋₆ alkenyl, and C₂₋₆ alkynyl, each of which is optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₃₋₁₀ carbocycle, and 3- to 10-membered heterocycle; and
   C₃₋₁₀ carbocycle, and 3- to 10-membered heterocycle, each of which is optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, 3- to 10-membered heterocycle, and C₁₋₆haloalkyl.

In some embodiments, each w is independently selected from any value from 1 to 10. In some embodiments, each w is independently selected from any value from 1 to 5. In some embodiments, each w is 1. In some embodiments, each v is independently selected from any value from 1 to 10. In some embodiments, each v is independently selected from any value from 1 to 5. In some embodiments, each v is 1. In some embodiments, n is selected from any value from 1 to 10. In some embodiments, n is selected from any value from 1 to 5. In some embodiments, n is 2. In some embodiments, m is selected from any value from 1 to 10. In some embodiments, m is selected from any value from 1 to 5. In some embodiments, m is selected from 1 and 2. In some embodiments, z is 3 and Y is C. In some embodiments, Q is selected from C₅₋₆ carbocycle optionally substituted with one or more substituents independently selected from halogen, —CN, —NO₂, —OR⁷, —SR⁷, —N(R⁷)₂, —C(O)R⁷, —C(O)N(R⁷)₂, —N(R⁷)C(O)R⁷, —N(R⁷)C(O)N(R⁷)₂, —OC(O)N(R⁷)₂, —N(R⁷)C(O)OR⁷, —C(O)OR⁷, —OC(O)R⁷, and —S(O)R⁷. In some embodiments, Q is selected from C₅₋₆ carbocycle optionally substituted with one or more substituents independently selected from halogen, —CN, —OH, —SH, —NO₂, and —NH₂. In some embodiments, Q is selected from phenyl and cyclohexyl, each of which is optionally substituted with one or more substituents independently selected from halogen, —CN, —OH, —SH, —NO₂, and —NH₂. In some embodiments, Q is selected from phenyl. In some embodiments, Q is selected from cyclohexyl. In some embodiments, R¹ is selected from —OP(O)(OR⁷)O—, —SP(O)(OR⁷)O—, —OP(S)(OR⁷)O—, —OP(O)(SR⁷)O—, —OP(O)(OR⁷)S—, —OP(O)(O⁻)O—, —SP(O)(O⁻)O—, —OP(S)(O⁻)O—, —OP(O)(S⁻)O—, —OP(O)(O⁻)S—, —OP(O)(OR⁷)NR⁷—, —OP(O)(N(R⁷)₂)NR⁷—, —OP(OR⁷)O—, —OP(N(R⁷)₂)O—, —OP(OR⁷)N(R⁷)—, and —OPN(R⁷)₂, NR⁷. In some embodiments, R¹ is selected from —OP(O)(OR⁷)O—, —SP(O)(OR⁷)O—, —OP(S)(OR⁷)O—, —OP(O)(SR⁷)O—, —OP(O)(OR⁷)S—, —OP(O)(O⁻)O—, —SP(O)(O⁻)O—, —OP(S)(O⁻)O—, —OP(O)(S⁻)O—, —OP(O)(O⁻)S—, and —OP(OR⁷)O—. In some embodiments, R¹ is selected from —OP(O)(OR⁷)O—, —OP(S)(OR⁷)O—, —OP(O)(O⁻)O—, —OP(S)(O⁻)O—, —OP(O)(S⁻)O—, and —OP(OR⁷)O—. In some embodiments, R¹ is selected from —OP(O)(OR⁷)O— and —OP(OR⁷)O—. In some embodiments, R² is selected from C₁₋₃ alkyl substituted with one or more substituents independently selected from halogen, —OR⁷, —OC(O)R⁷, —SR⁷, —N(R⁷)₂, —C(O)R⁷, and —S(O)R⁷. In some embodiments, R² is selected from C₁₋₃ alkyl substituted with one or more substituents independently selected from —OR⁷, —OC(O)R⁷, —SR⁷, and —N(R⁷)₂. In some embodiments, R² is selected from C₁₋₃ alkyl substituted with one or more substituents independently selected from —OR⁷ and —OC(O)R⁷. In some embodiments, R³ is selected from halogen, —OR⁷, —SR⁷, —N(R⁷)₂, —C(O)R⁷, —OC(O)R⁷, and —S(O)R⁷. In some embodiments, R³ is selected from —OR⁷, —SR⁷, —OC(O)R⁷, and —N(R⁷)₂. In some embodiments, R³ is selected from —OR⁷— and —OC(O)R⁷. In some embodiments, R⁴ is selected from halogen, —OR⁷, —SR⁷, —N(R⁷)₂, —C(O)R⁷, —OC(O)R⁷, and —S(O)R⁷. In some embodiments, R⁴ is selected from —OR⁷, —SR⁷, —OC(O)R⁷, and —N(R⁷)₂. In some embodiments, R⁴ is selected from —OR⁷— and —OC(O)R⁷. In some embodiments, R⁵ is selected from —OC(O)R⁷, —OC(O)N(R⁷)₂, —N(R⁷)C(O)R⁷, —N(R⁷)C(O)N(R⁷)₂, and —N(R⁷)C(O)OR⁷. In some embodiments, R⁵ is selected from —OC(O)R⁷ and —N(R⁷)C(O)R⁷. In some embodiments, each R⁷ is independently selected from: hydrogen; and C₁₋₆ alkyl optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₃₋₁₀ carbocycle, or 3- to 10-membered heterocycle. In some embodiments, each R⁷ is independently selected from C₁₋₆ alkyl optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, and —NH(C₁₋₆ alkyl). In some embodiments, each R⁷ is independently selected from C₁₋₆ alkyl optionally substituted with one or more substituents independently selected from halogen, —CN, —OH, and —SH. In some embodiments, w is 1; v is 1; n is 2; m is 1 or 2; z is 3 and Y is C; Q is phenyl or cyclohexyl, each of which is optionally substituted with one or more substituents independently selected from halogen, —CN, —OH, —SH, —NO₂, —NH₂, and C₁₋₃ alkyl; R¹ is selected from —OP(O)(OR⁷)O—, —OP(S)(OR⁷)O—, —OP(O)(O⁻)O—, —OP(S)(O⁻)O—, —OP(O)(S⁻)O—, and —OP(OR⁷)O—; R² is C₁ alkyl substituted with —OH or —OC(O)CH₃;
R³ is —OH or —OC(O)CH₃; R⁴ is —OH or —OC(O)CH₃; and R⁵ is —NH(O)CH₃. In some embodiments, the compound comprises:

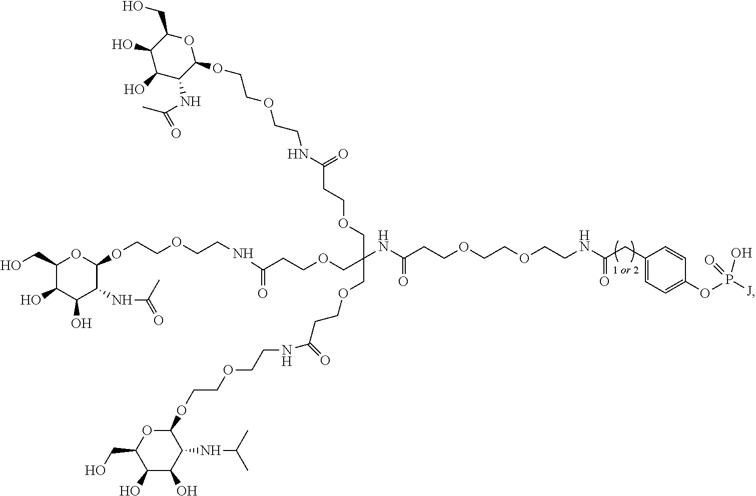

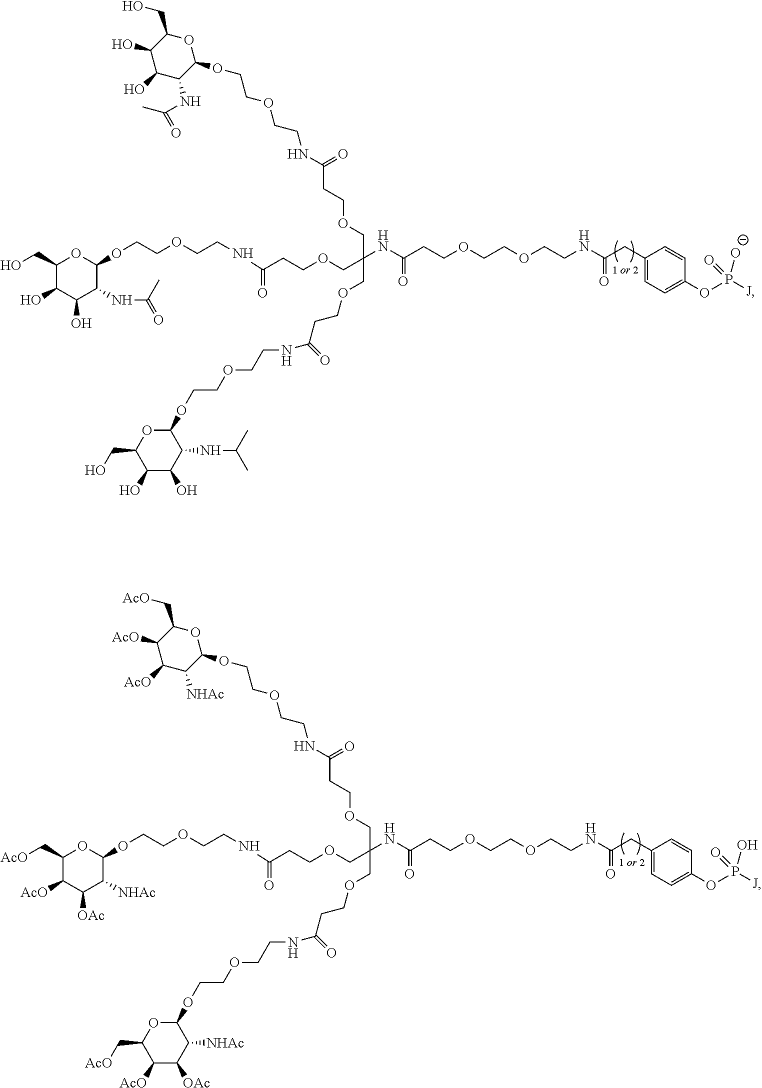

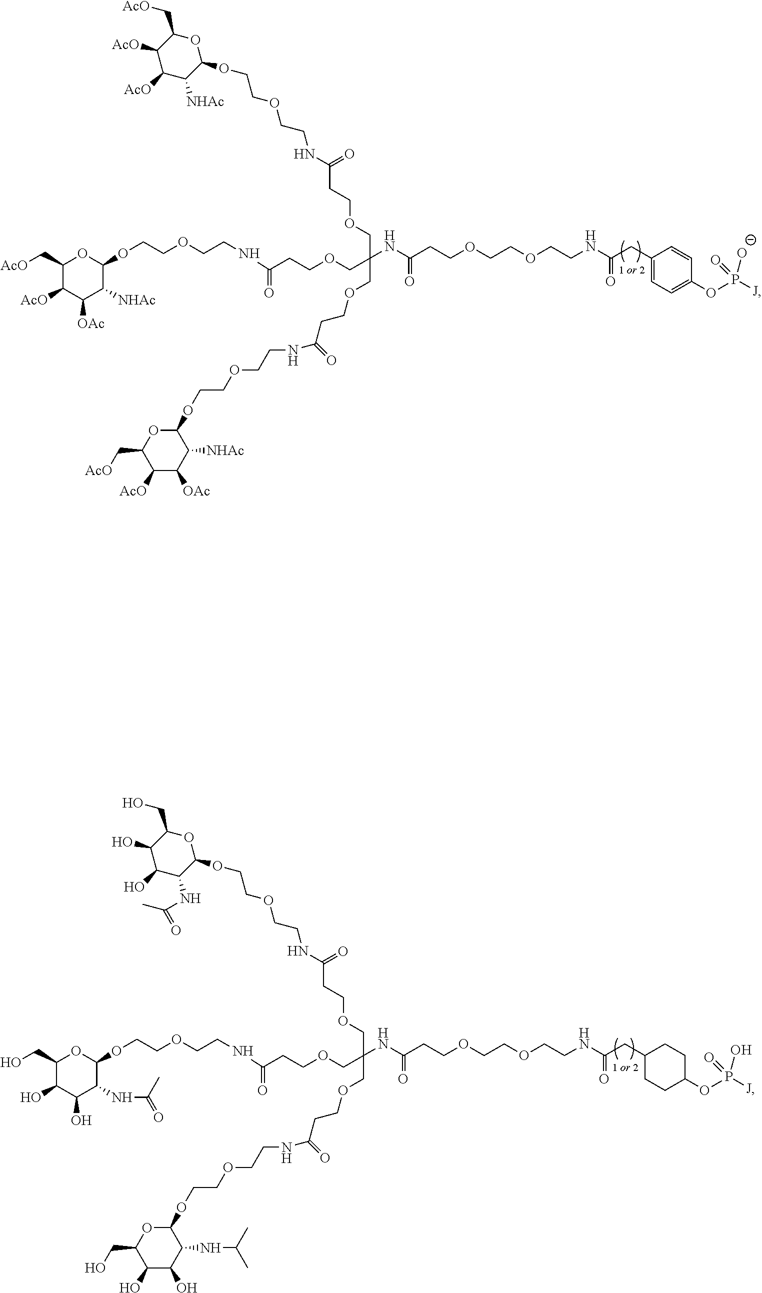

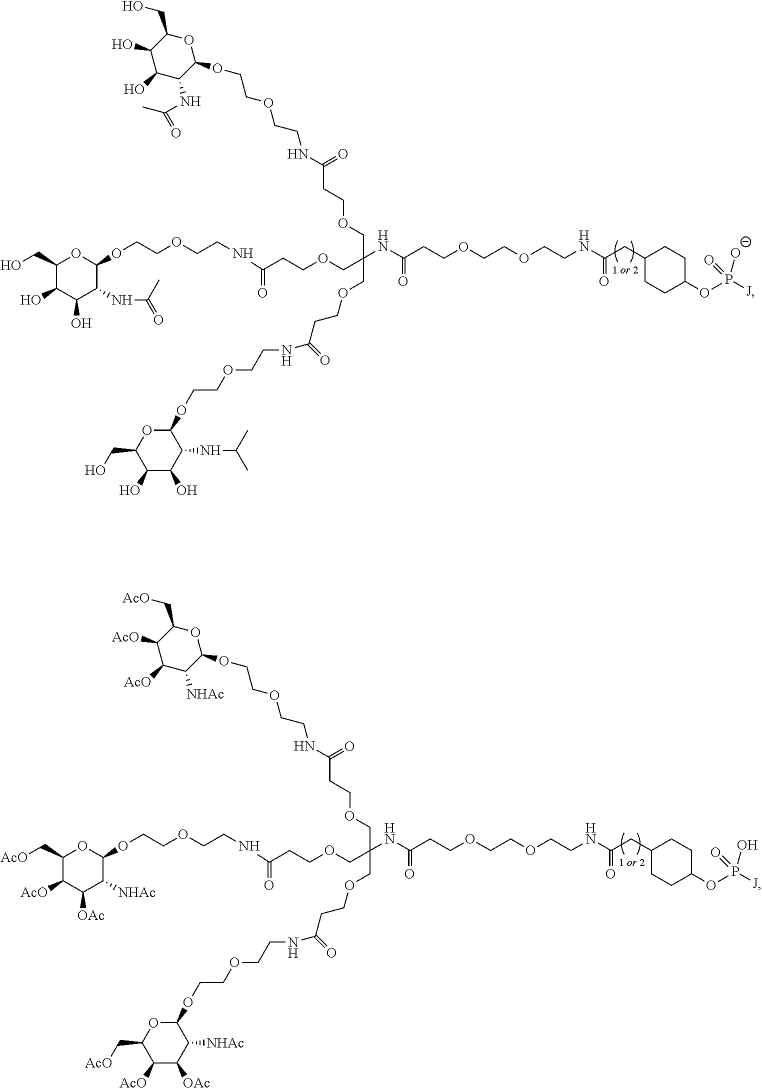

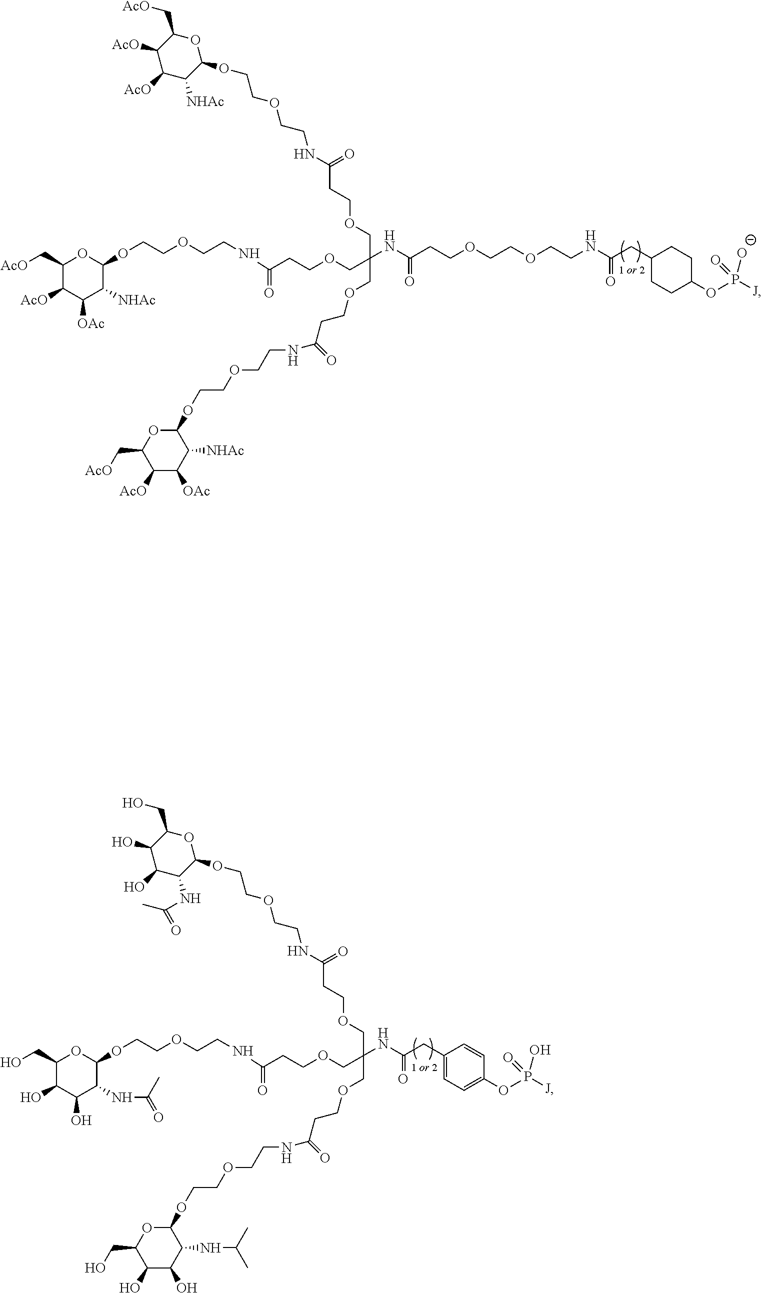

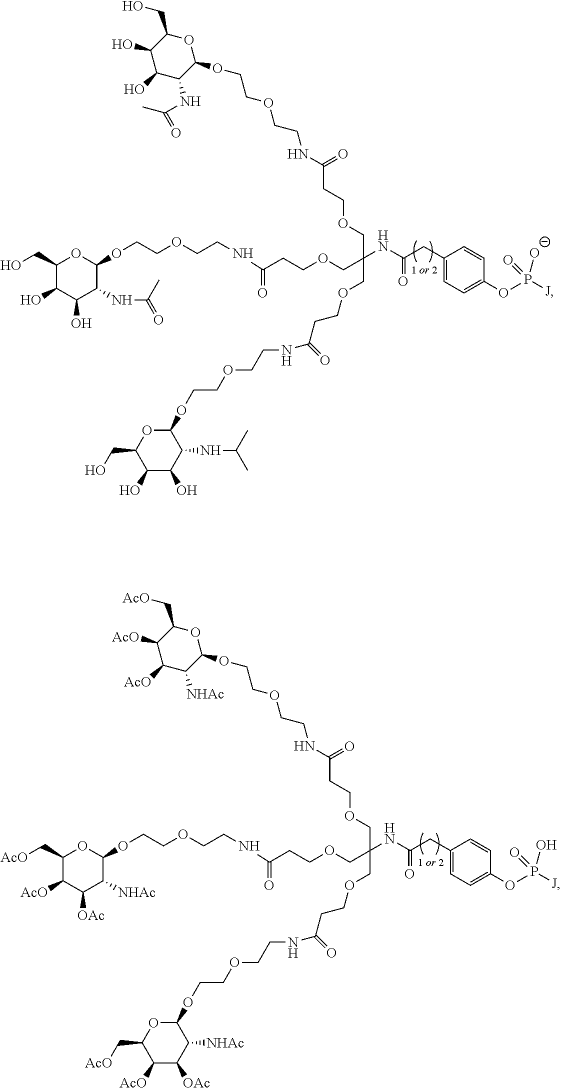

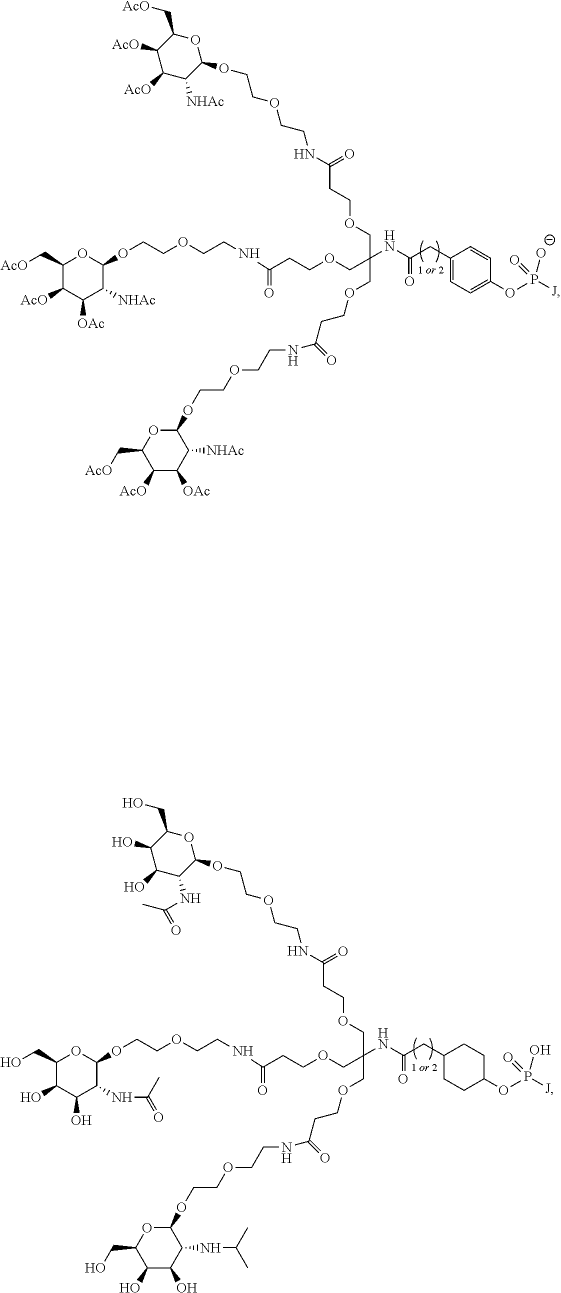

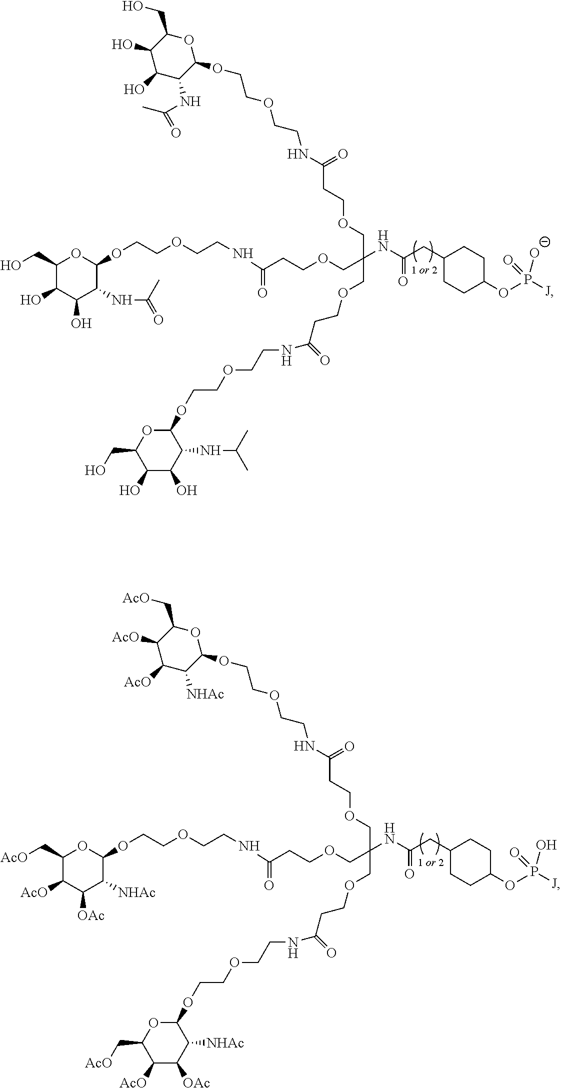

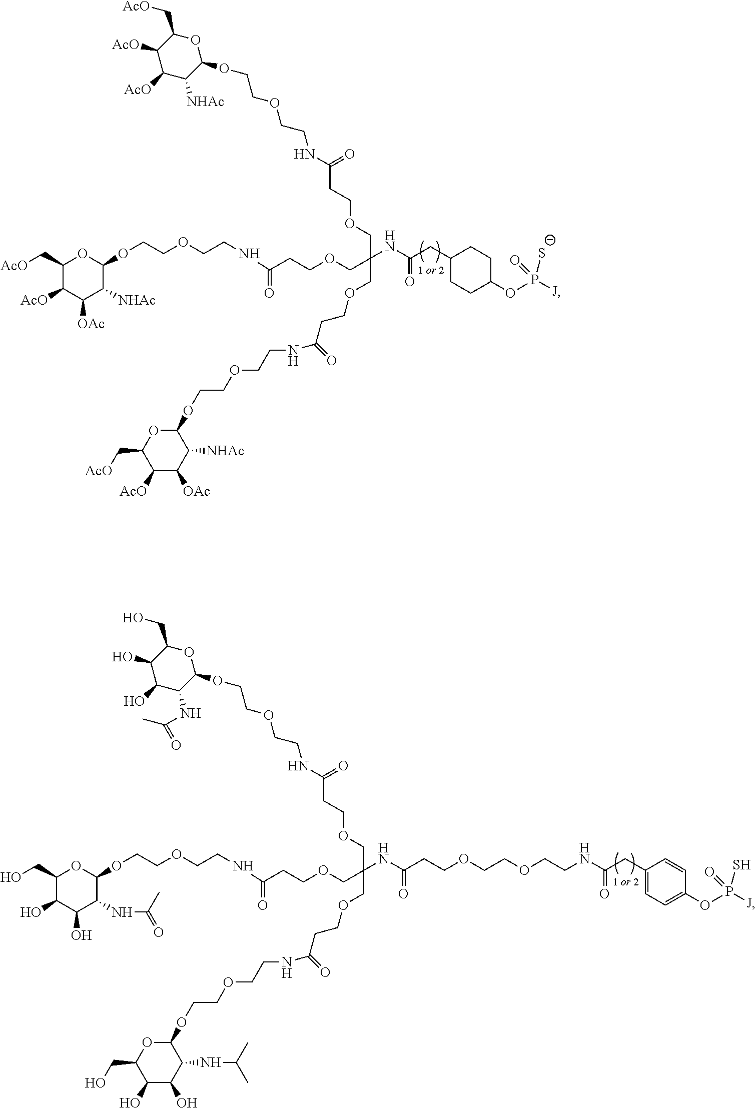

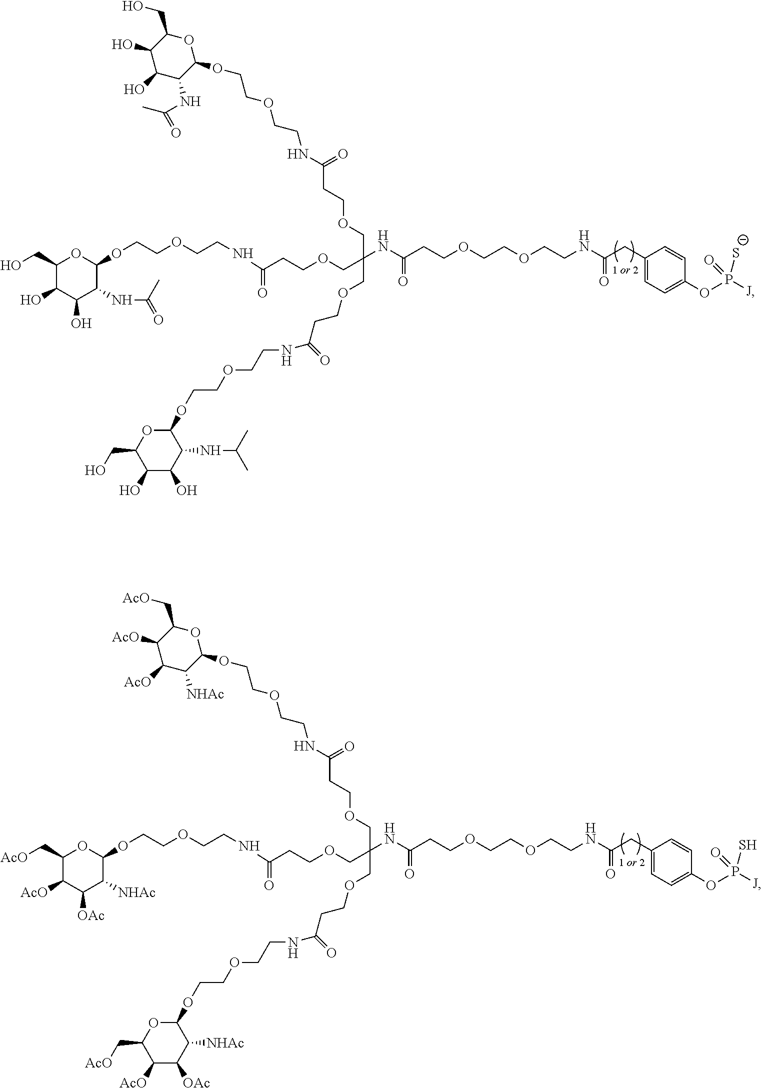

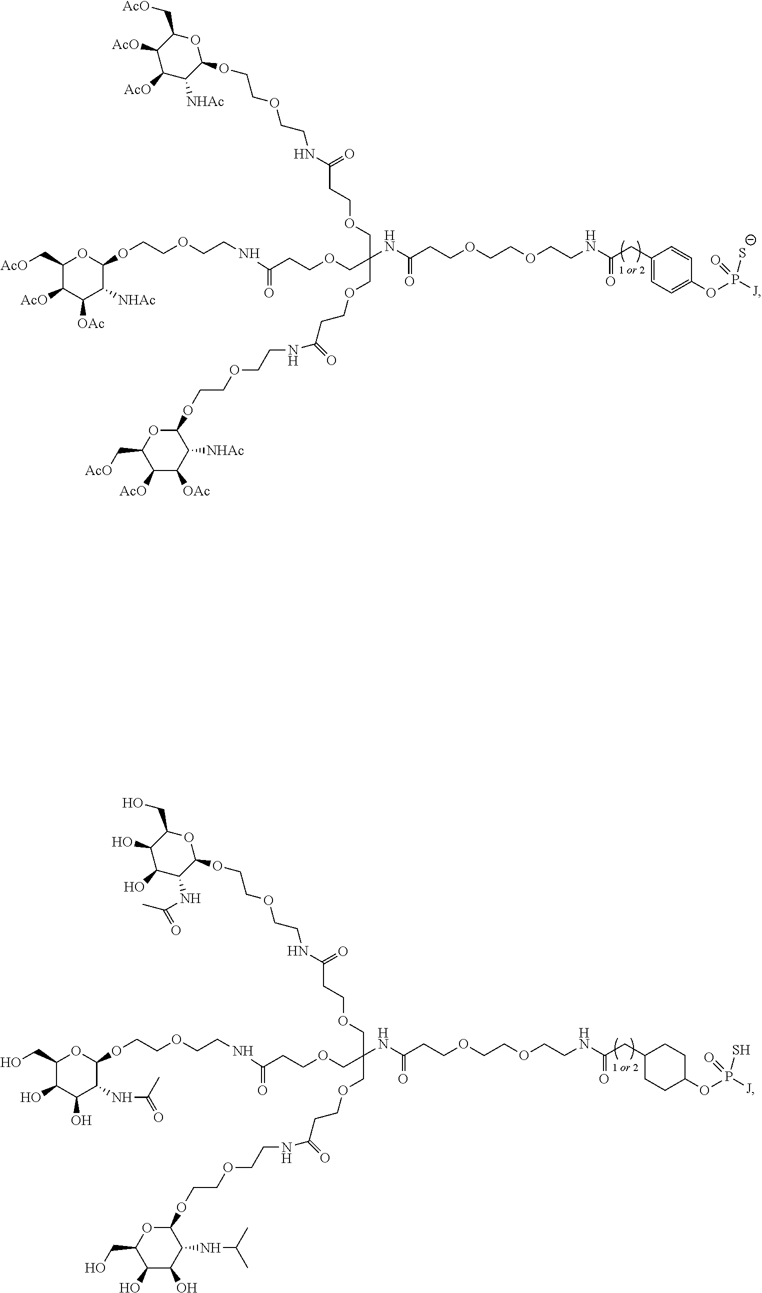

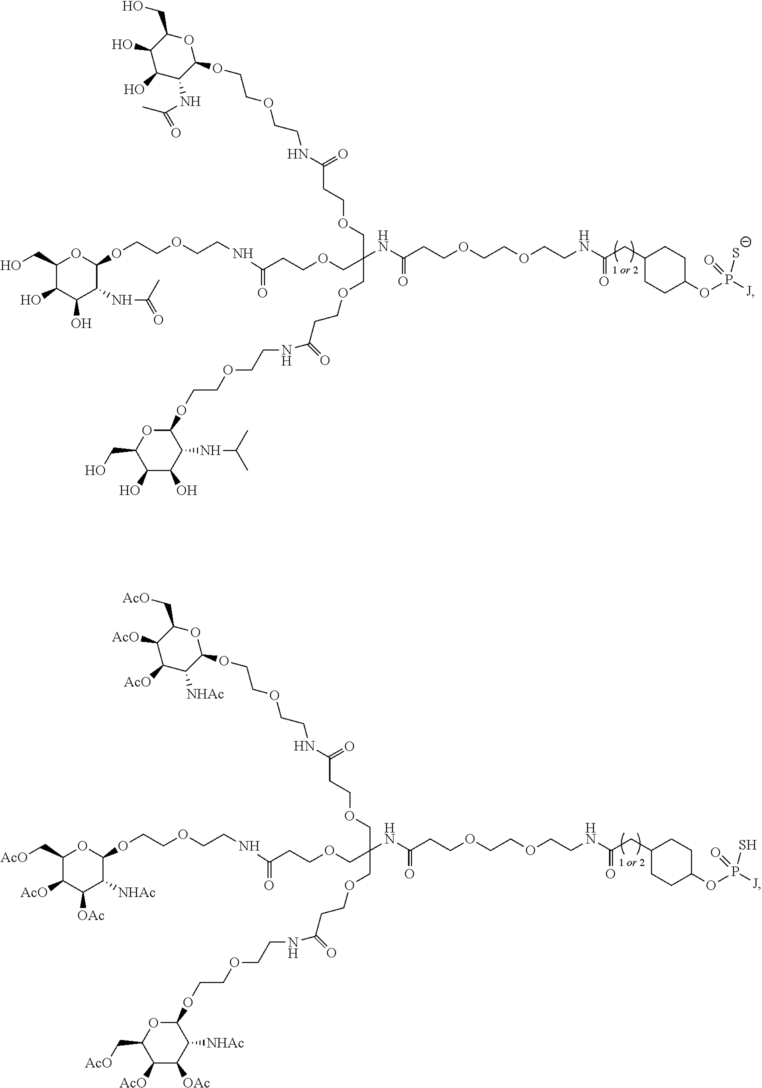

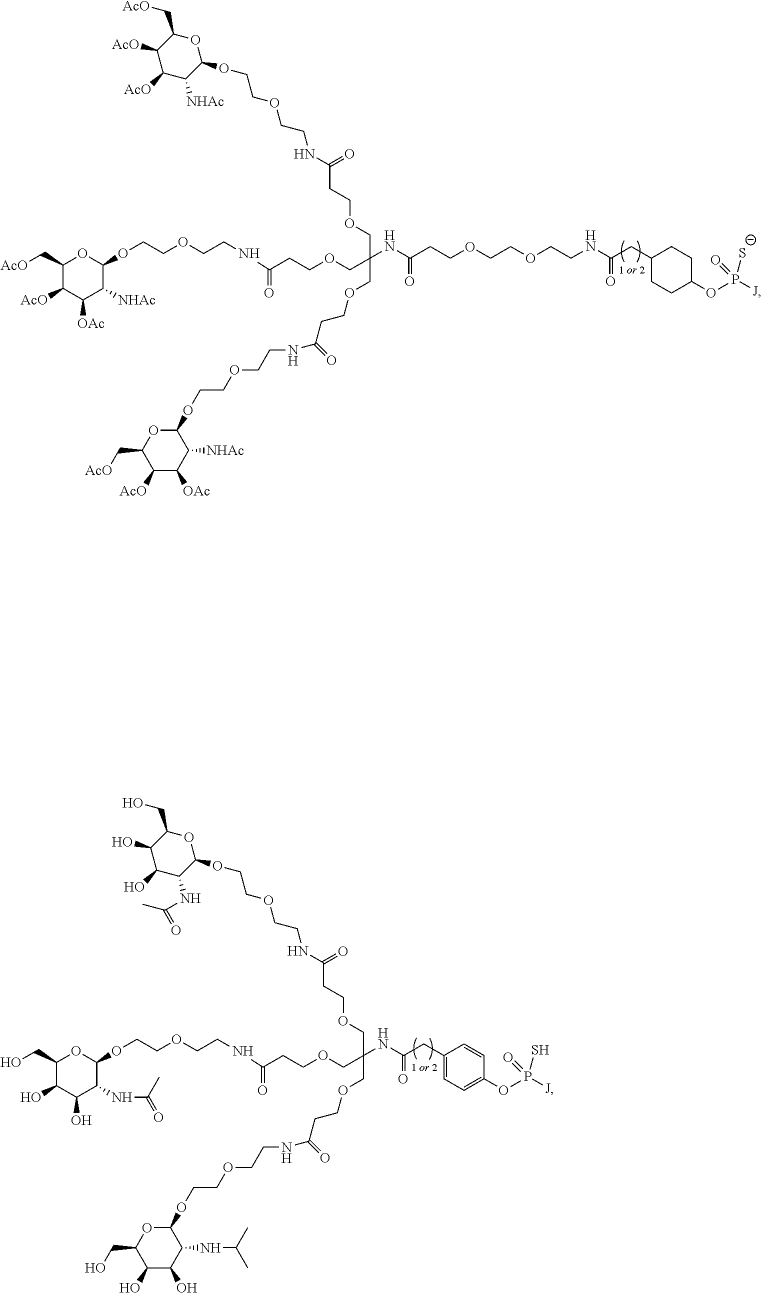

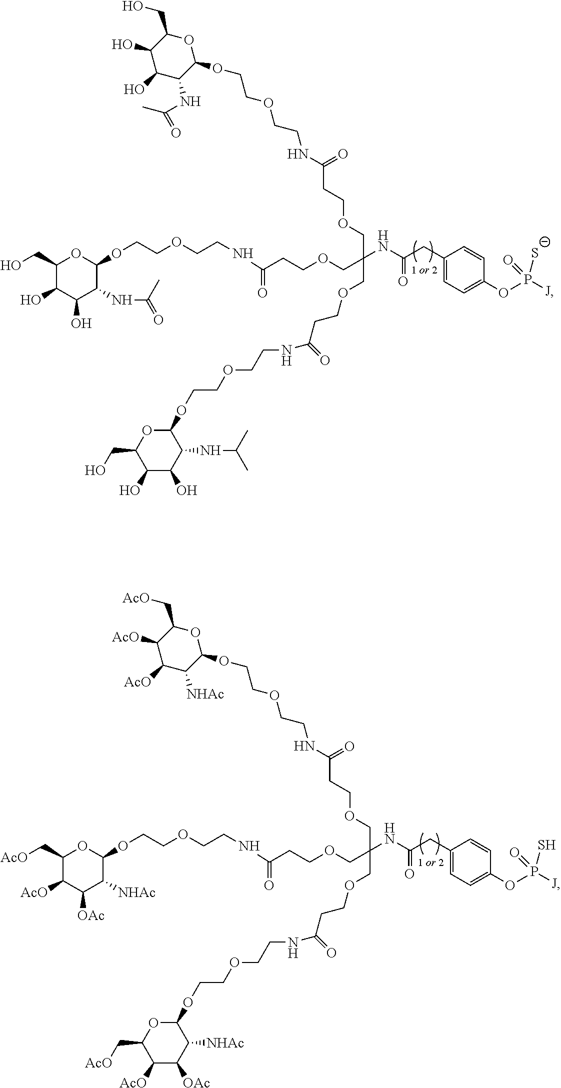

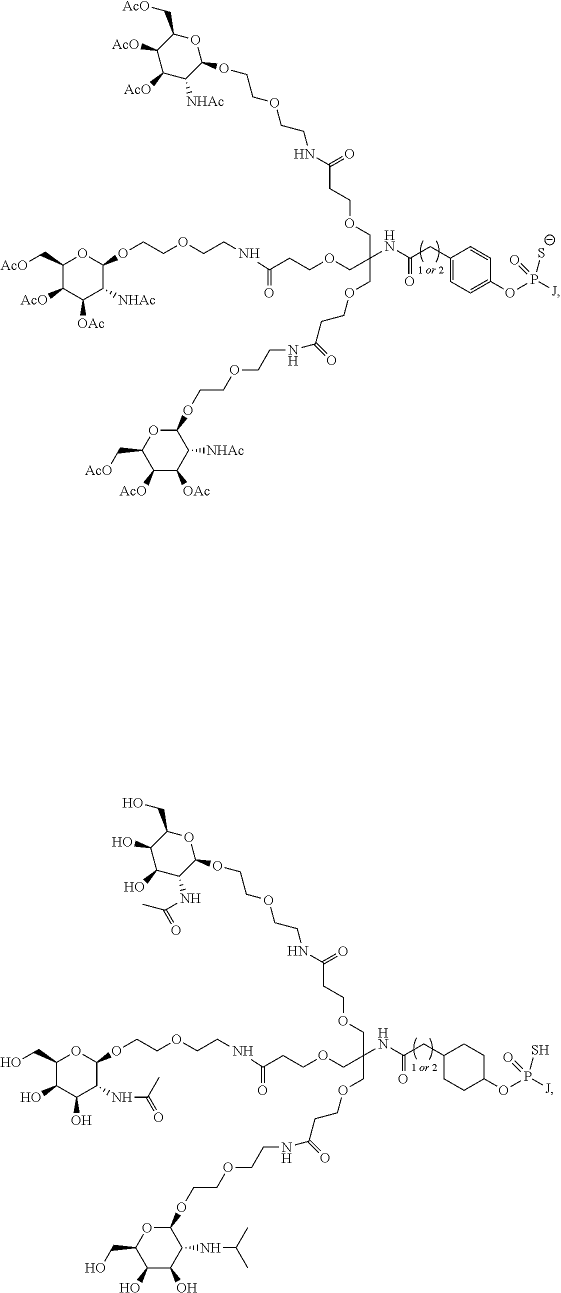

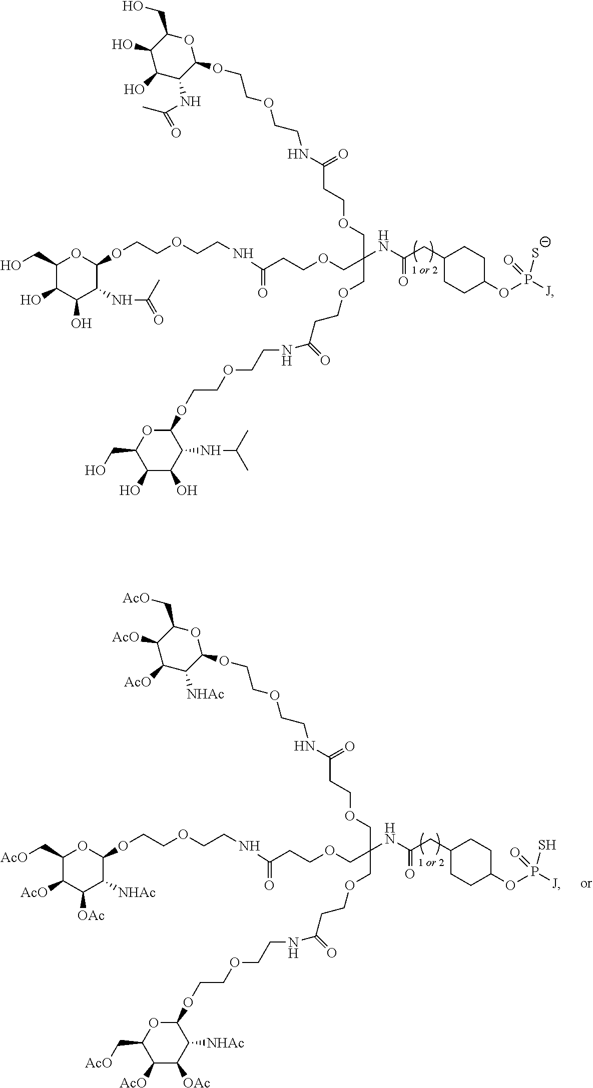

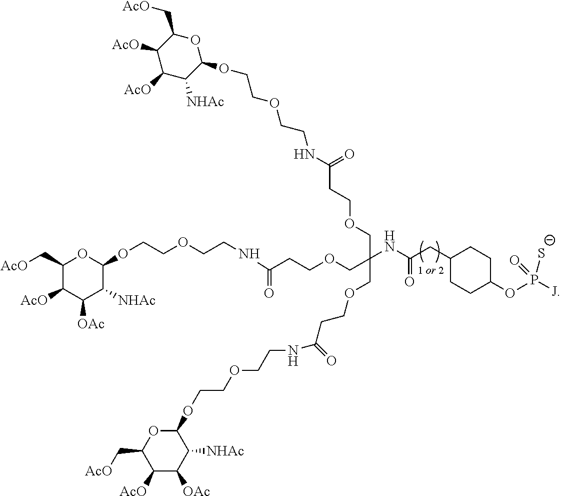

In some embodiments, the oligonucleotide (J) is attached at a 5′ end or a 3′ end of the oligonucleotide. In some embodiments, the oligonucleotide comprises DNA. In some embodiments, the oligonucleotide comprises RNA. In some embodiments, the oligonucleotide comprises one or more modified internucleoside linkages. In some embodiments, the one or more modified internucleoside linkages comprise alkylphosphonate, phosphorothioate, methylphosphonate, phosphorodithioate, alkylphosphonothioate, phosphoramidate, carbamate, carbonate, phosphate triester, acetamidate, or carboxymethyl ester, or a combination thereof. In some embodiments, the oligonucleotide comprises 1, 2, 3, 4, 5, 6, 7, 8, 9, 10, 11, 12, 13, 14, 15, 16, 17, 18, 19, or 20 modified internucleoside linkages. In some embodiments, the compound binds to an asialoglycoprotein receptor. In some embodiments, the compound targets a hepatocyte.

Some embodiments include the following, where J is the oligonucleotide:

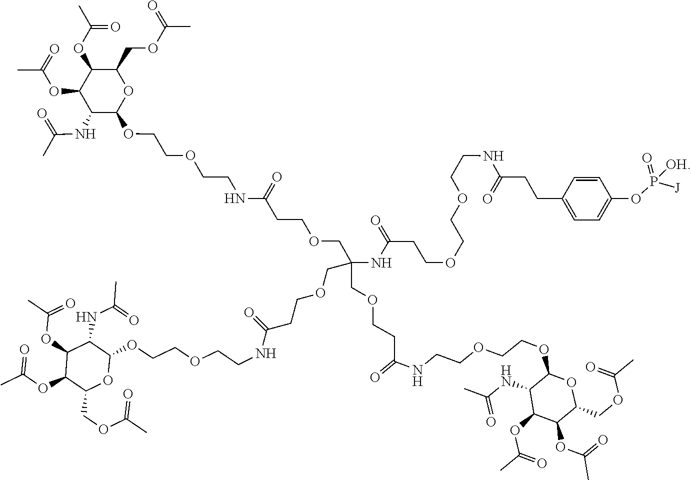

J may include one or more additional phosphates, or one or more phosphorothioates linking to the oligonucleotide. J may include one or more additional phosphates linking to the oligonucleotide. J may include one or more phosphorothioates linking to the oligonucleotide.

Some embodiments include the following, where J is the oligonucleotide:

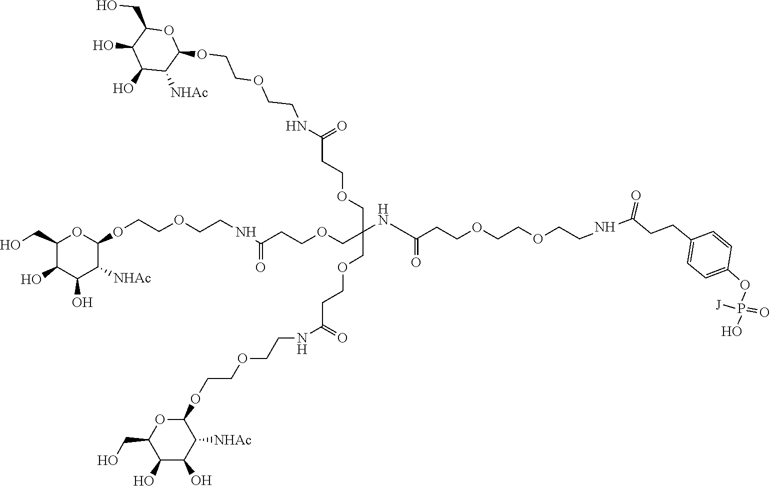

J may include one or more additional phosphates, or one or more phosphorothioates linking to the oligonucleotide. J may include one or more additional phosphates linking to the oligonucleotide. J may include one or more phosphorothioates linking to the oligonucleotide.

Some embodiments include the following, where J is the oligonucleotide:

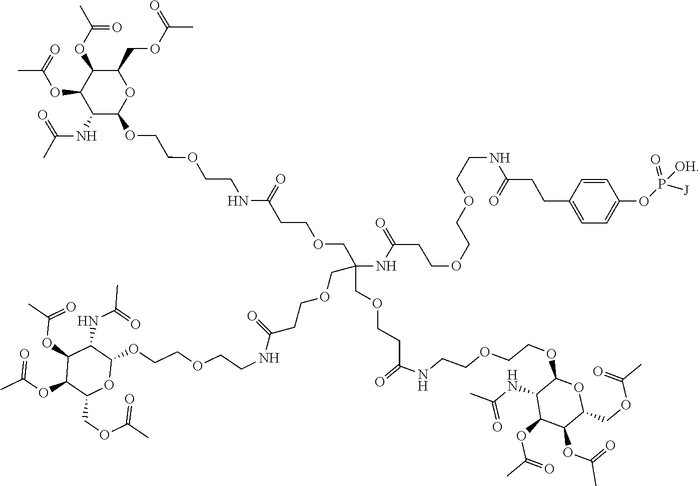

J may include one or more phosphates or phosphorothioates linking to the oligonucleotide. J may include one or more phosphates linking to the oligonucleotide. J may include a phosphate linking to the oligonucleotide. J may include one or more phosphorothioates linking to the oligonucleotide. J may include a phosphorothioate linking to the oligonucleotide.

Some embodiments include the following, where J is the oligonucleotide:

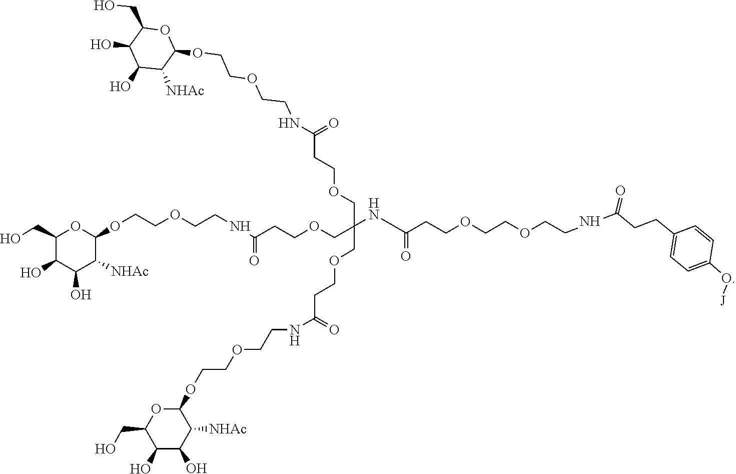

The structure in this compound attached to the oligonucleotide (J) may be referred to as “ETL17,” and is an example of a GalNAc moiety. J may include one or more phosphates or phosphorothioates linking to the oligonucleotide. J may include one or more phosphates linking to the oligonucleotide. J may include a phosphate linking to the oligonucleotide. J may include one or more phosphorothioates linking to the oligonucleotide. J may include a phosphorothioate linking to the oligonucleotide.

Some embodiments include the following, where the phosphate or “5′” indicates a connection to the oligonucleotide:

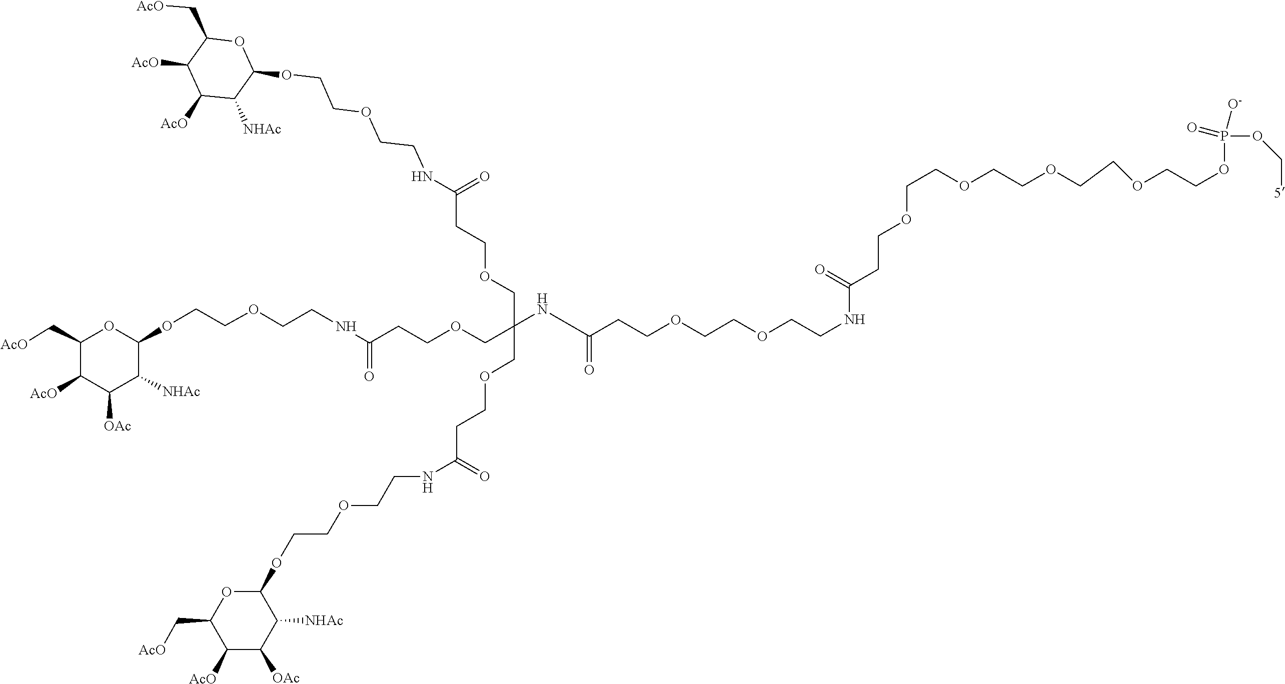

Some embodiments include the following, where the phosphate or “5′” indicates a connection to the oligonucleotide:

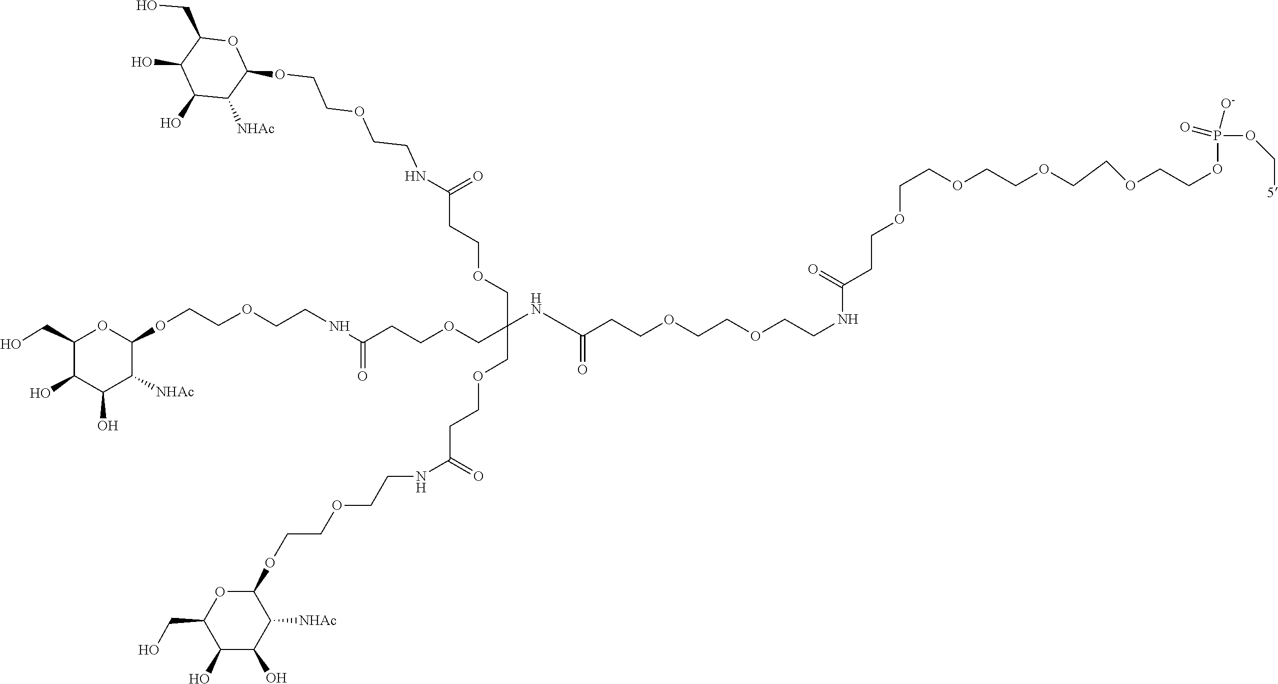

Some embodiments include the following, where J is the oligonucleotide:

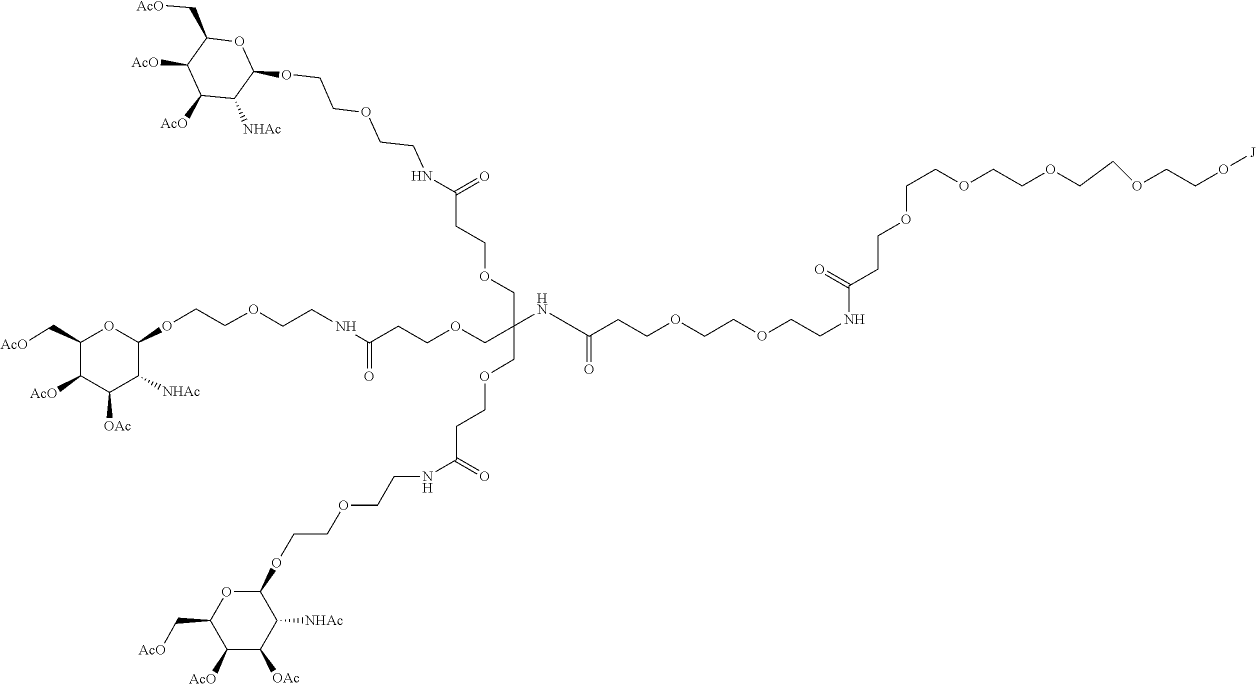

include one or more phosphates or phosphorothioates linking to the oligonucleotide. J may include one or more phosphates linking to the oligonucleotide. J may include a phosphate linking to the oligonucleotide. J may include one or more phosphorothioates linking to the oligonucleotide. J may include a phosphorothioate linking to the oligonucleotide.

Some embodiments include the following, where J is the oligonucleotide:

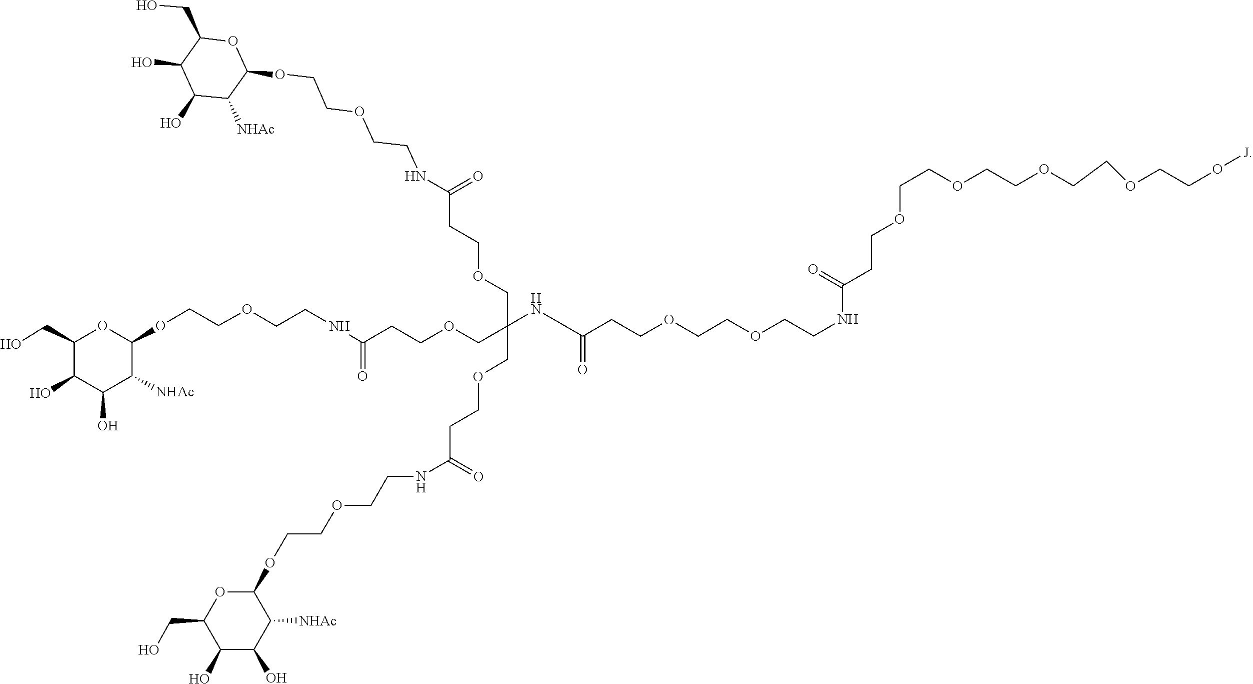

The structure in this compound attached to the oligonucleotide (J) may be referred to as “ETL1,” and is an example of a GalNAc moiety. J may include one or more phosphates or phosphorothioates linking to the oligonucleotide. J may include one or more phosphates linking to the oligonucleotide. J may include a phosphate linking to the oligonucleotide. J may include one or more phosphorothioates linking to the oligonucleotide. J may include a phosphorothioate linking to the oligonucleotide.
3. siRNA Modification Patterns

In some embodiments, the composition comprises an oligonucleotide that inhibits the expression of FGG, wherein the oligonucleotide comprises an siRNA comprising a sense strand and an antisense strand, wherein the sense strand comprises modification pattern 1S: 5′-NfsnsNfnNfnNfNfNfnNfnNfnNfnNfnNfsnsn-3′ (SEQ ID NO: 3622), wherein “Nf” is a 2′ fluoro-modified nucleoside, “n” is a 2′ O-methyl modified nucleoside, and “s” is a phosphorothioate linkage. In some embodiments, the sense strand comprises modification pattern 2S: 5′-nsnsnnNfnNfNfNfnnnnnnnnnnsnsn-3′ (SEQ ID NO: 3623), wherein “Nf” is a 2′ fluoro-modified nucleoside, “n” is a 2′ O-methyl modified nucleoside, and “s” is a phosphorothioate linkage. In some embodiments, the sense strand comprises modification pattern 3S: 5′-nsnsnnNfnNfnNfnnnnnnnnnnsnsn-3′ (SEQ ID NO: 3624), wherein “Nf” is a 2′ fluoro-modified nucleoside, “n” is a 2′ O-methyl modified nucleoside, and “s” is a phosphorothioate linkage. In some embodiments, the sense strand comprises modification pattern 4S: 5′-NfsnsNfnNfnNfNfNfnNfnNfnNfnNfnNfsnsnN-moiety-3′ (SEQ ID NO: 3625), wherein “Nf” is a 2′ fluoro-modified nucleoside, “n” is a 2′ O-methyl modified nucleoside, “s” is a phosphorothioate linkage, and N comprises one or more nucleosides. In some embodiments, the sense strand comprises modification pattern 5S: 5′-nsnsnnNfnNfNfNfnnnnnnnnnnsnsnN-moiety-3′ (SEQ ID NO: 3626), wherein “Nf” is a 2′ fluoro-modified nucleoside, “n” is a 2′ O-methyl modified nucleoside, “s” is a phosphorothioate linkage, and N comprises one or more nucleosides. In some embodiments, the moiety in modification pattern 4S or 5S is a lipid moiety. In some embodiments, the moiety in modification pattern 4S or 5S is a sugar moiety. In some embodiments, the sense strand comprises modification pattern 6S: 5′-NfsnsNfnNfnNfnNfnNfnNfnNfnNfnNfsnsn-3′ (SEQ ID NO: 3627), wherein “Nf” is a 2′ fluoro-modified nucleoside, “n” is a 2′ O-methyl modified nucleoside, and “s” is a phosphorothioate linkage. In some embodiments, the sense strand comprises modification pattern 7S: 5′-nsnsnnNfNfNfNfNfnnnnnnnnnnsnsn-3′ (SEQ ID NO: 3628), wherein “Nf” is a 2′ fluoro-modified nucleoside, “n” is a 2′ O-methyl modified nucleoside, and “s” is a phosphorothioate linkage. In some embodiments, the sense strand comprises modification pattern 8S: 5′-nsnsnnnNfNfNfNfnnnnnnnnnnsnsn-3′ (SEQ ID NO: 3629), wherein “Nf” is a 2′ fluoro-modified nucleoside, “n” is a 2′ O-methyl modified nucleoside, and “s” is a phosphorothioate linkage. In some embodiments, the sense strand comprises modification pattern 9S: 5′-nsnsnnnnNfNfNfNfnnnnnnnnnsnsn-3′ (SEQ ID NO: 3630), wherein “Nf” is a 2′ fluoro-modified nucleoside, “n” is a 2′ O-methyl modified nucleoside, and “s” is a phosphorothioate linkage. In some embodiments, the sense strand comprises modification pattern 10S: 5′-nsnsnnNfNfnNfNfnnnnnnnnnnsnsn-3′ (SEQ ID NO: 3785), wherein “Nf” is a 2′ fluoro-modified nucleoside, “n” is a 2′ O-methyl modified nucleoside, and “s” is a phosphorothioate linkage.

In some embodiments, the sense strand comprises modification pattern 11S: 5′-nsnsnnNfnnnNfnnnnnnnnnnsnsn-3′ (SEQ ID NO: 3786), wherein “Nf” is a 2′ fluoro-modified nucleoside, “n” is a 2′ O-methyl modified nucleoside, and “s” is a phosphorothioate linkage.

In some embodiments, the sense strand comprises modification pattern 12S: 5′-snnnnNfNfnNfNfnnnnNfnnNfnnsnsn-3′ (SEQ ID NO: 3787), wherein “Nf” is a 2′ fluoro-modified nucleoside, “n” is a 2′ O-methyl modified nucleoside, and “s” is a phosphorothioate linkage.

In some embodiments, the sense strand comprises modification pattern 13S: 5′-snnnnNfNfnNfdNnNfNfnnNfnnnnsnsn-3′ (SEQ ID NO: 3788), wherein “Nf” is a 2′ fluoro-modified nucleoside, “n” is a 2′ O-methyl modified nucleoside, and “s” is a phosphorothioate linkage.

In some embodiments, the sense strand comprises modification pattern 14S: 5′-snnNfNfnnnnNfnnnnNfnNfNfnnsnsn-3′ (SEQ ID NO: 3789), wherein “Nf” is a 2′ fluoro-modified nucleoside, “n” is a 2′ O-methyl modified nucleoside, and “s” is a phosphorothioate linkage.

In some embodiments, the sense strand comprises modification pattern 15S: 5′-snnNfnNfnNfNfdNnNfNfnnNfnnnnsnsn-3′ (SEQ ID NO: 3790), wherein “Nf” is a 2′ fluoro-modified nucleoside, “n” is a 2′ O-methyl modified nucleoside, and “s” is a phosphorothioate linkage. In some embodiments, the sense strand comprises modification pattern 16S: 5′-snnnnNfnNfNfNfNfnnnnnnnnnsnsn-3′ (SEQ ID NO: 3791), wherein “Nf” is a 2′ fluoro-modified nucleoside, “n” is a 2′ O-methyl modified nucleoside, and “s” is a phosphorothioate linkage. In some embodiments, the sense strand comprises modification pattern 17S: 5′-snnnnnNfNfNfNfnnnnnnnnnnsnsn-3′ (SEQ ID NO: 3792), wherein “Nf” is a 2′ fluoro-modified nucleoside, “n” is a 2′ O-methyl modified nucleoside, and “s” is a phosphorothioate linkage. In some embodiments, the sense strand comprises modification pattern 18S: 5′-snnnnNfNfnNfNfnnnnnnnnnnsnsn-3′ (SEQ ID NO: 3793), wherein “Nf” is a 2′ fluoro-modified nucleoside, “n” is a 2′ O-methyl modified nucleoside, and “s” is a phosphorothioate linkage. In some embodiments, the sense strand comprises modification pattern 19S: 5′-snnnnNfnnnNfnnnnnnnnnnsnsn-3′ (SEQ ID NO: 3794), wherein “Nf” is a 2′ fluoro-modified nucleoside, “n” is a 2′ O-methyl modified nucleoside, and “s” is a phosphorothioate linkage. In some embodiments, the sense strand comprises modification pattern 20S: 5′-snnnnnNfNfNfNfnNfnnnnnnnnsnsn-3′ (SEQ ID NO: 3795), wherein “Nf” is a 2′ fluoro-modified nucleoside, “n” is a 2′ O-methyl modified nucleoside, and “s” is a phosphorothioate linkage. In some embodiments, the sense strand comprises modification pattern 21S: 5′-snnnnnnNfNfNfNfNfnnnnnnnnsnsn-3′ (SEQ ID NO: 3796), wherein “Nf” is a 2′ fluoro-modified nucleoside, “n” is a 2′ O-methyl modified nucleoside, and “s” is a phosphorothioate linkage. In some embodiments, the sense strand comprises modification pattern 22S: 5′-snnnnNfNfnNfNfnNfnnnnnnnnsnsn-3′ (SEQ ID NO: 3797), wherein “Nf” is a 2′ fluoro-modified nucleoside, “n” is a 2′ O-methyl modified nucleoside, and “s” is a phosphorothioate linkage. In some embodiments, the sense strand comprises modification pattern 23S: 5′-snnnnNfnNfNfdTnnnnnnnnnnsnsn-3′ (SEQ ID NO: 3798), wherein “dT” is deoxythymidine, “Nf” is a 2′ fluoro-modified nucleoside, “n” is a 2′ O-methyl modified nucleoside, and “s” is a phosphorothioate linkage. In some embodiments, the sense strand comprises modification pattern 24S: 5′-snnnnNfNfnnNfNfnnnnnnnnnsnsn-3′ (SEQ ID NO: 3799), wherein “Nf” is a 2′ fluoro-modified nucleoside, “n” is a 2′ O-methyl modified nucleoside, and “s” is a phosphorothioate linkage. In some embodiments, the sense strand comprises modification pattern 25S: 5′-snnnnnNfNfnNfnnnnnnnnnnsnsn-3′ (SEQ ID NO: 3800), wherein “Nf” is a 2′ fluoro-modified nucleoside, “n” is a 2′ O-methyl modified nucleoside, and “s” is a phosphorothioate linkage. In some embodiments, the sense strand comprises modification pattern 26S: 5′-snnnnnnNfnNfNfnnnnnnnnnsnsn-3′ (SEQ ID NO: 3801), wherein “Nf” is a 2′ fluoro-modified nucleoside, “n” is a 2′ O-methyl modified nucleoside, and “s” is a phosphorothioate linkage. In some embodiments, the sense strand comprises modification pattern 27S: 5′-snnnnnnnNfNfnNfnnnnnnnnsnsn-3′ (SEQ ID NO: 3802), wherein “Nf” is a 2′ fluoro-modified nucleoside, “n” is a 2′ O-methyl modified nucleoside, and “s” is a phosphorothioate linkage. In some embodiments, the sense strand comprises modification pattern 28S: 5′-snnnnnnnnNfnNfnNfnmnnnnsnsn-3′ (SEQ ID NO: 3803), wherein “Nf” is a 2′ fluoro-modified nucleoside, “n” is a 2′ O-methyl modified nucleoside, and “s” is a phosphorothioate linkage. In some embodiments, the sense strand comprises modification pattern 29S: 5′-snnnnnnnNfNfNfNfnnnnnnnnsnsn-3′ (SEQ ID NO: 3804), wherein “Nf” is a 2′ fluoro-modified nucleoside, “n” is a 2′ O-methyl modified nucleoside, and “s” is a phosphorothioate linkage.

In some embodiments, the composition comprises an oligonucleotide that inhibits the expression of FGG, wherein the oligonucleotide comprises an siRNA comprising a sense strand and an antisense strand, wherein the antisense strand comprises modification pattern 1AS: 5′-nsNfsnNfnNfnNfnNfnnnNfnNfnNfnsnsn-3′ (SEQ ID NO: 3631), wherein “Nf” is a 2′ fluoro-modified nucleoside, “n” is a 2′ O-methyl modified nucleoside, and “s” is a phosphorothioate linkage. In some embodiments, the antisense strand comprises modification pattern 2AS: 5′-nsNfsnnnNfnNfNfnnnnNfnNfnnnsnsn-3′ (SEQ ID NO: 3632), wherein “Nf” is a 2′ fluoro-modified nucleoside, “n” is a 2′ O-methyl modified nucleoside, and “s” is a phosphorothioate linkage. In some embodiments, the antisense strand comprises modification pattern 3AS: 5′-nsNfsnnnNfnnnnnnnNfnNfnnnsnsn-3′ (SEQ ID NO: 3633), wherein “Nf” is a 2′ fluoro-modified nucleoside, “n” is a 2′ O-methyl modified nucleoside, and “s” is a phosphorothioate linkage. In some embodiments, the antisense strand comprises modification pattern 4AS: 5′-nsNfsnNfnNfnnnnnnnNfnNfnnnsnsn-3′ (SEQ ID NO: 3634), wherein “Nf” is a 2′ fluoro-modified nucleoside, “n” is a 2′ O-methyl modified nucleoside, and “s” is a phosphorothioate linkage. In some embodiments, the antisense strand comprises modification pattern 5AS: 5′-nsNfsnnnnnnnnnnnNfnNfnnnsnsn-3′ (SEQ ID NO: 3635), wherein “Nf” is a 2′ fluoro-modified nucleoside, “n” is a 2′ O-methyl modified nucleoside, and “s” is a phosphorothioate linkage. In some embodiments, the antisense strand comprises modification pattern 6AS: 5′-nsNfsnnnNfnnNfnnnnNfnNfnnnsnsn-3′ (SEQ ID NO: 3636), wherein “Nf” is a 2′ fluoro-modified nucleoside, “n” is a 2′ O-methyl modified nucleoside, and “s” is a phosphorothioate linkage. In some embodiments, the antisense strand comprises modification pattern 7AS: 5′-nsNfsnNfnNfnNfnNfnNfnNfnNfnNfnsnsn-3′ (SEQ ID NO: 3637), wherein “Nf” is a 2′ fluoro-modified nucleoside, “n” is a 2′ O-methyl modified nucleoside, and “s” is a phosphorothioate linkage. In some embodiments, the antisense strand comprises modification pattern 8AS: 5′-nsNfsnnnnnnnnnnnNfnnnnnsnsn-3′ (SEQ ID NO: 3638), wherein “Nf” is a 2′ fluoro-modified nucleoside, “n” is a 2′ O-methyl modified nucleoside, and “s” is a phosphorothioate linkage. In some embodiments, the antisense strand comprises modification pattern 9AS: 5′-nNfnNfnNfnNfnNfnNfnNfnNfnNfnsnsn-3′ (SEQ ID NO: 3639), wherein “Nf” is a 2′ fluoro-modified nucleoside, “n” is a 2′ O-methyl modified nucleoside, and “s” is a phosphorothioate linkage. In some embodiments, the antisense strand comprises modification pattern 10AS: 5′-nsNfsnNfnnnNfnNfnNfnNfnNfnNfnsnsn-3′ (SEQ ID NO: 3805), wherein “Nf” is a 2′ fluoro-modified nucleoside, “n” is a 2′ O-methyl modified nucleoside, and “s” is a phosphorothioate linkage. In some embodiments, the antisense strand comprises modification pattern 11AS: 5′-nsNfsnNfnnNfnnNfnNfnNfnNfnNfnsnsn-3′ (SEQ ID NO: 3806), wherein “Nf” is a 2′ fluoro-modified nucleoside, “n” is a 2′ O-methyl modified nucleoside, and “s” is a phosphorothioate linkage. In some embodiments, the antisense strand comprises modification pattern 12AS: 5′-nsNfsndTndNnNfnNfndNnNfndNnNfnsnsn-3′ (SEQ ID NO: 3807), wherein “Nf” is a 2′ fluoro-modified nucleoside, “dT” is deoxythymidine, “n” is a 2′ O-methyl modified nucleoside, and “s” is a phosphorothioate linkage. In some embodiments, the antisense strand comprises modification pattern 13AS: 5′-nsNfsndTndNnNfnNfndNndTndNndTnsnsn-3′ (SEQ ID NO: 3808), wherein “Nf” is a 2′ fluoro-modified nucleoside, “dT” is deoxythymidine, “n” is a 2′ O-methyl modified nucleoside, and “s” is a phosphorothioate linkage. In some embodiments, the antisense strand comprises modification pattern 14AS: 5′-nsNfsnnnNfnnnNfnNfnNfnNfnNfnsnsn-3′ (SEQ ID NO: 3809), wherein “Nf” is a 2′ fluoro-modified nucleoside, “n” is a 2′ O-methyl modified nucleoside, and “s” is a phosphorothioate linkage. In some embodiments, the antisense strand comprises modification pattern 15AS: 5′-dTsNfsnnnNfnNfnNfnNfnNfnNfnNfnsnsn-3′ (SEQ ID NO: 3810), wherein “Nf” is a 2′ fluoro-modified nucleoside, “dT” is deoxythymidine, “n” is a 2′ O-methyl modified nucleoside, and “s” is a phosphorothioate linkage. In some embodiments, the antisense strand comprises modification pattern 16As: 5′-NfsNfsnnnNfnNfnNfnNfnNfnNfnNfnsnsn-3′ (SEQ ID NO: 3811), wherein “Nf” is a 2′ fluoro-modified nucleoside, “n” is a 2′ O-methyl modified nucleoside, and “s” is a phosphorothioate linkage. In some embodiments, the antisense strand comprises modification pattern 17AS: 5′-nsNfsnnnNfnNfnNfnNfnNfnNfnNfnsnsn-3′ (SEQ ID NO: 3812), wherein “Nf” is a 2′ fluoro-modified nucleoside, “n” is a 2′ O-methyl modified nucleoside, and “s” is a phosphorothioate linkage. In some embodiments, the antisense strand comprises modification pattern 18AS: 5′-nsNfsnNfnNfnnnNfnNfnNfnNfnNfnsnsn-3′ (SEQ ID NO: 3813), wherein “Nf” is a 2′ fluoro-modified nucleoside, “n” is a 2′ O-methyl modified nucleoside, and “s” is a phosphorothioate linkage. In some embodiments, the antisense strand comprises modification pattern 19AS: 5′-nsNfsnNfnnNfNfnNfnNfnNfnNfnNfnsnsn-3′ (SEQ ID NO: 3814), wherein “Nf” is a 2′ fluoro-modified nucleoside, “n” is a 2′ O-methyl modified nucleoside, and “s” is a phosphorothioate linkage. In some embodiments, the antisense strand comprises modification pattern 20AS: 5′-nsNfsnNfnnNfnnnnNfnNfnNfnNfnsnsn-3′ (SEQ ID NO: 3815), wherein “Nf” is a 2′ fluoro-modified nucleoside, “n” is a 2′ O-methyl modified nucleoside, and “s” is a phosphorothioate linkage. In some embodiments, the antisense strand comprises modification pattern 21AS: 5′-nsNfsnnnNfnNfnnnNfnNfnNfnNfnsnsn-3′ (SEQ ID NO: 3816), wherein “Nf” is a 2′ fluoro-modified nucleoside, “n” is a 2′ O-methyl modified nucleoside, and “s” is a phosphorothioate linkage.

In some embodiments, the composition comprises an oligonucleotide that inhibits the expression of FGG, wherein the oligonucleotide comprises an siRNA comprising a sense strand and an antisense strand, wherein the sense strand comprises pattern 1S and the antisense strand comprises pattern 1AS, 2AS, 3AS, 4AS, 5AS, 6AS, 7AS, 8AS, 9AS, 10AS, 11AS, 12AS, 13AS, 14AS, 15AS, 16AS, 17AS, 18AS, 19AS, 20AS, or 21AS. In some embodiments, the sense strand comprises pattern 2S and the antisense strand comprises pattern 1AS, 2AS, 3AS, 4AS, 5AS, 6AS, 7AS, 8AS, 9AS, 10AS, 11AS, 12AS, 13AS, 14AS, 15AS, 16AS, 17AS, 18AS, 19AS, 20AS, or 21AS. In some embodiments, the sense strand comprises pattern 3S and the antisense strand comprises pattern 1AS, 2AS, 3AS, 4AS, 5AS, 6AS, 7AS, 8AS, 9AS, 10AS, 11AS, 12AS, 13AS, 14AS, 15AS, 16AS, 17AS, 18AS, 19AS, 20AS, or 21AS. In some embodiments, the sense strand comprises pattern 4S and the antisense strand comprises pattern 1AS, 2AS, 3AS, 4AS, 5AS, 6AS, 7AS, 8AS, 9AS, 10AS, 11AS, 12AS, 13AS, 14AS, 15AS, 16AS, 17AS, 18AS, 19AS, 20AS, or 21AS. In some embodiments, the sense strand comprises pattern 5S and the antisense strand comprises pattern 1AS, 2AS, 3AS, 4AS, 5AS, 6AS, 7AS, 8AS, 9AS, 10AS, 11AS, 12AS, 13AS, 14AS, 15AS, 16AS, 17AS, 18AS, 19AS, 20AS, or 21AS. In some embodiments, the sense strand comprises pattern 6S and the antisense strand comprises pattern 1AS, 2AS, 3AS, 4AS, 5AS, 6AS, 7AS, 8AS, 9AS, 10AS, 11AS, 12AS, 13AS, 14AS, 15AS, 16AS, 17AS, 18AS, 19AS, 20AS, or 21AS. In some embodiments, the sense strand comprises pattern 7S and the antisense strand comprises pattern 1AS, 2AS, 3AS, 4AS, 5AS, 6AS, 7AS, 8AS, 9AS, 10AS, 11AS, 12AS, 13AS, 14AS, 15AS, 16AS, 17AS, 18AS, 19AS, 20AS, or 21AS. In some embodiments, the sense strand comprises pattern 8S and the antisense strand comprises pattern 1AS, 2AS, 3AS, 4AS, 5AS, 6AS, 7AS, 8AS, 9AS, 10AS, 11AS, 12AS, 13AS, 14AS, 15AS, 16AS, 17AS, 18AS, 19AS, 20AS, or 21AS. In some embodiments, the sense strand comprises pattern 9S and the antisense strand comprises pattern 1AS, 2AS, 3AS, 4AS, 5AS, 6AS, 7AS, 8AS, 9AS, 10AS, 11AS, 12AS, 13AS, 14AS, 15AS, 16AS, 17AS, 18AS, 19AS, 20AS, or 21AS. In some embodiments, the sense strand comprises pattern 10S and the antisense strand comprises pattern 1AS, 2AS, 3AS, 4AS, 5AS, 6AS, 7AS, 8AS, 10AS, 11AS, 12AS, 13AS, 14AS, 15AS, 16AS, 17AS, 18AS, 19AS, 20AS, or 21AS. In some embodiments, the sense strand comprises pattern 11S and the antisense strand comprises pattern 1AS, 2AS, 3AS, 4AS, 5AS, 6AS, 7AS, 8AS, 10AS, 11AS, 12AS, 13AS, 14AS, 15AS, 16AS, 17AS, 18AS, 19AS, 20AS, or 21AS. In some embodiments, the sense strand comprises pattern 12S and the antisense strand comprises pattern 1AS, 2AS, 3AS, 4AS, 5AS, 6AS, 7AS, 8AS, 10AS, 11AS, 12AS, 13AS, 14AS, 15AS, 16AS, 17AS, 18AS, 19AS, 20AS, or 21AS. In some embodiments, the sense strand comprises pattern 13S and the antisense strand comprises pattern 1AS, 2AS, 3AS, 4AS, 5AS, 6AS, 7AS, 8AS, 10AS, 11AS, 12AS, 13AS, 14AS, 15AS, 16AS, 17AS, 18AS, 19AS, 20AS, or 21AS. In some embodiments, the sense strand comprises pattern 14S and the antisense strand comprises pattern 1AS, 2AS, 3AS, 4AS, 5AS, 6AS, 7AS, 8AS, 10AS, 11AS, 12AS, 13AS, 14AS, 15AS, 16AS, 17AS, 18AS, 19AS, 20AS, or 21AS. In some embodiments, the sense strand comprises pattern 15S and the antisense strand comprises pattern 1AS, 2AS, 3AS, 4AS, 5AS, 6AS, 7AS, 8AS, 10AS, 11AS, 12AS, 13AS, 14AS, 15AS, 16AS, 17AS, 18AS, 19AS, 20AS, or 21AS. In some embodiments, the sense strand comprises pattern 16S and the antisense strand comprises pattern 1AS, 2AS, 3AS, 4AS, 5AS, 6AS, 7AS, 8AS, 10AS, 11AS, 12AS, 13AS, 14AS, 15AS, 16AS, 17AS, 18AS, 19AS, 20AS, or 21AS. In some embodiments, the sense strand comprises pattern 17S and the antisense strand comprises pattern 1AS, 2AS, 3AS, 4AS, 5AS, 6AS, 7AS, 8AS, 10AS, 11AS, 12AS, 13AS, 14AS, 15AS, 16AS, 17AS, 18AS, 19AS, 20AS, or 21AS. In some embodiments, the sense strand comprises pattern 18S and the antisense strand comprises pattern 1AS, 2AS, 3AS, 4AS, 5AS, 6AS, 7AS, 8AS, 10AS, 11AS, 12AS, 13AS, 14AS, 15AS, 16AS, 17AS, 18AS, 19AS, 20AS, or 21AS. In some embodiments, the sense strand comprises pattern 19S and the antisense strand comprises pattern 1AS, 2AS, 3AS, 4AS, 5AS, 6AS, 7AS, 8AS, 10AS, 11AS, 12AS, 13AS, 14AS, 15AS, 16AS, 17AS, 18AS, 19AS, 20AS, or 21AS. In some embodiments, the sense strand comprises pattern 20S and the antisense strand comprises pattern 1AS, 2AS, 3AS, 4AS, 5AS, 6AS, 7AS, 8AS, 10AS, 11AS, 12AS, 13AS, 14AS, 15AS, 16AS, 17AS, 18AS, 19AS, 20AS, or 21AS. In some embodiments, the sense strand comprises pattern 21S and the antisense strand comprises pattern 1AS, 2AS, 3AS, 4AS, 5AS, 6AS, 7AS, 8AS, 10AS, 11AS, 12AS, 13AS, 14AS, 15AS, 16AS, 17AS, 18AS, 19AS, 20AS, or 21AS. In some embodiments, the sense strand comprises pattern 22S and the antisense strand comprises pattern 1AS, 2AS, 3AS, 4AS, 5AS, 6AS, 7AS, 8AS, 10AS, 11AS, 12AS, 13AS, 14AS, 15AS, 16AS, 17AS, 18AS, 19AS, 20AS, or 21AS. In some embodiments, the sense strand comprises pattern 23S and the antisense strand comprises pattern 1AS, 2AS, 3AS, 4AS, 5AS, 6AS, 7AS, 8AS, 10AS, 11AS, 12AS, 13AS, 14AS, 15AS, 16AS, 17AS, 18AS, 19AS, 20AS, or 21AS. In some embodiments, the sense strand comprises pattern 24S and the antisense strand comprises pattern 1AS, 2AS, 3AS, 4AS, 5AS, 6AS, 7AS, 8AS, 10AS, 11AS, 12AS, 13AS, 14AS, 15AS, 16AS, 17AS, 18AS, 19AS, 20AS, or 21AS. In some embodiments, the sense strand comprises pattern 25S and the antisense strand comprises pattern 1AS, 2AS, 3AS, 4AS, 5AS, 6AS, 7AS, 8AS, 10AS, 11AS, 12AS, 13AS, 14AS, 15AS, 16AS, 17AS, 18AS, 19AS, 20AS, or 21AS. In some embodiments, the sense strand comprises pattern 26S and the antisense strand comprises pattern 1AS, 2AS, 3AS, 4AS, 5AS, 6AS, 7AS, 8AS, 10AS, 11AS, 12AS, 13AS, 14AS, 15AS, 16AS, 17AS, 18AS, 19AS, 20AS, or 21AS. In some embodiments, the sense strand comprises pattern 27S and the antisense strand comprises pattern 1AS, 2AS, 3AS, 4AS, 5AS, 6AS, 7AS, 8AS, 10AS, 11AS, 12AS, 13AS, 14AS, 15AS, 16AS, 17AS, 18AS, 19AS, 20AS, or 21AS. In some embodiments, the sense strand comprises pattern 28S and the antisense strand comprises pattern 1AS, 2AS, 3AS, 4AS, 5AS, 6AS, 7AS, 8AS, 10AS, 11AS, 12AS, 13AS, 14AS, 15AS, 16AS, 17AS, 18AS, 19AS, 20AS, or 21AS. In some embodiments, the sense strand comprises pattern 29S and the antisense strand comprises pattern 1AS, 2AS, 3AS, 4AS, 5AS, 6AS, 7AS, 8AS, 10AS, 11AS, 12AS, 13AS, 14AS, 15AS, 16AS, 17AS, 18AS, 19AS, 20AS, or 21AS.

In some embodiments, the sense strand comprises pattern 1S, 2S, 3S, 4S, 5S, 6S, 7S, 8S, 9S, 10S, 11S, 12S, 13S, 14S, 15S, 156S, 17S, 18S, 19S, 20S, 21S, 22S, 23S, 24S, 25S, 28S, or 29S and the antisense strand comprises pattern 1AS. In some embodiments, the sense strand comprises pattern 1S, 2S, 3S, 4S, 5S, 6S, 7S, 8S, 9S, 10S, 11S, 12S, 13S, 14S, 15S, 156S, 17S, 18S, 19S, 20S, 21S, 22S, 23S, 24S, 25S, 28S, or 29S and the antisense strand comprises pattern 2AS. In some embodiments, the sense strand comprises pattern 1S, 2S, 3S, 4S, 5S, 6S, 7S, 8S, 9S, 10S, 11S, 12S, 13S, 14S, 15S, 156S, 17S, 18S, 19S, 20S, 21S, 22S, 23S, 24S, 25S, 28S, or 29S and the antisense strand comprises pattern 3AS. In some embodiments, the sense strand comprises pattern 1S, 2S, 3S, 4S, 5S, 6S, 7S, 8S, 9S, 10S, 11S, 12S, 13S, 14S, 15S, 156S, 17S, 18S, 19S, 20S, 21S, 22S, 23S, 24S, 25S, 28S, or 29S and the antisense strand comprises pattern 4AS. In some embodiments, the sense strand comprises pattern 1S, 2S, 3S, 4S, 5S, 6S, 7S, 8S, 9S, 10S, 11S, 125, 135, 145, 155, 1565, 175, 185, 19S, 20S, 21S, 22S, 23S, 24S, 25S, 28S, or 29S and the antisense strand comprises pattern 5AS. In some embodiments, the sense strand comprises pattern 1S, 2S, 3S, 4S, 5S, 6S, 7S, 8S, 9S, 10S, 11S, 12S, 135, 14S, 15S, 156S, 17S, 18S, 19S, 20S, 21S, 22S, 23S, 24S, 25S, 28S, or 29S and the antisense strand comprises pattern 6AS. In some embodiments, the sense strand comprises pattern 1S, 2S, 3S, 4S, 5S, 6S, 7S, 8S, 9S, 10S, 11S, 12S, 13S, 14S, 15S, 156S, 17S, 18S, 19S, 20S, 21S, 22S, 23S, 24S, 25S, 28S, or 29S and the antisense strand comprises pattern 7AS. In some embodiments, the sense strand comprises pattern 1S, 2S, 3S, 4S, 5S, 6S, 7S, 8S, 9S, 10S, 11S, 12S, 13S, 14S, 15S, 156S, 17S, 18S, 19S, 20S, 21S, 22S, 23S, 24S, 25S, 28S, or 29S and the antisense strand comprises pattern 8AS. In some embodiments, the sense strand comprises pattern 1S, 2S, 3S, 4S, 5S, 6S, 7S, 8S, 9S, 10S, 11S, 12S, 13S, 14S, 15S, 156S, 17S, 18S, 19S, 20S, 21S, 22S, 23S, 24S, 25S, 28S, or 29S and the antisense strand comprises pattern 9AS. In some embodiments, the sense strand comprises pattern 1S, 2S, 3S, 4S, 5S, 6S, 7S, 8S, 9S, 10S, 11S, 12S, 13S, 14S, 15S, 156S, 17S, 18S, 19S, 20S, 21S, 22S, 23S, 24S, 25S, 28S, or 29S and the antisense strand comprises pattern 10AS. In some embodiments, the sense strand comprises pattern 1S, 2S, 3S, 4S, 5S, 6S, 7S, 8S, 9S, 10S, 11S, 12S, 13S, 14S, 15S, 156S, 17S, 18S, 19S, 20S, 21S, 22S, 23S, 24S, 25S, 28S, or 29S and the antisense strand comprises pattern 11AS. In some embodiments, the sense strand comprises pattern 1S, 2S, 3S, 4S, 5S, 6S, 7S, 8S, 9S, 10S, 11S, 12S, 13S, 14S, 155, 156S, 17S, 18S, 19S, 20S, 21S, 22S, 23S, 24S, 25S, 28S, or 29S and the antisense strand comprises pattern 12AS. In some embodiments, the sense strand comprises pattern 1S, 2S, 3S, 4S, 5S, 6S, 7S, 8S, 9S, 10S, 11S, 12S, 13S, 14S, 15S, 156S, 17S, 18S, 19S, 20S, 21S, 22S, 23S, 24S, 25S, 28S, or 29S and the antisense strand comprises pattern 13AS. In some embodiments, the sense strand comprises pattern 1S, 2S, 3S, 4S, 5S, 6S, 7S, 8S, 9S, 10S, 11S, 12S, 13S, 14S, 15S, 156S, 17S, 18S, 19S, 20S, 21S, 22S, 23S, 24S, 25S, 28S, or 29S and the antisense strand comprises pattern 14AS. In some embodiments, the sense strand comprises pattern 1S, 2S, 3S, 4S, 5S, 6S, 7S, 8S, 9S, 10S, 11S, 12S, 13S, 14S, 15S, 156S, 17S, 18S, 19S, 20S, 21S, 22S, 23S, 24S, 25S, 28S, or 29S and the antisense strand comprises pattern 15AS. In some embodiments, the sense strand comprises pattern 1S, 2S, 3S, 4S, 5S, 6S, 7S, 8S, 9S, 10S, 11S, 12S, 13S, 14S, 15S, 156S, 17S, 18S, 19S, 20S, 21S, 22S, 23S, 24S, 25S, 28S, or 29S and the antisense strand comprises pattern 16AS. In some embodiments, the sense strand comprises pattern 1S, 2S, 3S, 4S, 5S, 6S, 7S, 8S, 9S, 10S, 11S, 12S, 13S, 14S, 15S, 156S, 17S, 18S, 19S, 20S, 21S, 22S, 23S, 24S, 25S, 28S, or 29S and the antisense strand comprises pattern 17AS. In some embodiments, the sense strand comprises pattern 1S, 2S, 3S, 4S, 5S, 6S, 7S, 8S, 9S, 10S, 11S, 12S, 13S, 14S, 15S, 156S, 17S, 18S, 19S, 20S, 21S, 22S, 23S, 24S, 25S, 28S, or 29S and the antisense strand comprises pattern 18AS. In some embodiments, the sense strand comprises pattern 1S, 2S, 3S, 4S, 5S, 6S, 7S, 8S, 9S, 10S, 11S, 12S, 13S, 14S, 155, 156S, 17S, 18S, 19S, 20S, 21S, 22S, 23S, 24S, 255, 28S, or 29S and the antisense strand comprises pattern 19AS. In some embodiments, the sense strand comprises pattern 1S, 2S, 3S, 4S, 5S, 6S, 7S, 8S, 9S, 10S, 11S, 12S, 13S, 14S, 15S, 156S, 17S, 18S, 19S, 20S, 21S, 22S, 23S, 24S, 25S, 28S, or 29S and the antisense strand comprises pattern 209AS. In some embodiments, the sense strand comprises pattern 1S, 2S, 3S, 4S, 5S, 6S, 7S, 8S, 9S, 10S, 11S, 12S, 13S, 14S, 15S, 156S, 17S, 18S, 19S, 20S, 21S, 22S, 23S, 24S, 25S, 28S, or 29S and the antisense strand comprises pattern 21AS.

In some embodiments, the sense strand comprises modification pattern 1AS, 2AS, 3AS, 4AS, 5AS, 6AS, 7AS, 8AS, 9AS, 10AS, 11AS, 12AS, 13AS, 14AS, 15AS, 16AS, 17AS, 18AS, 19AS, 20AS, or 21AS. In some embodiments, the antisense strand comprises modification pattern 1S, 2S, 3S, 4S, 5S, 6S, 7S, 8S, 9S, 10S, 11S, 12S, 13S, 14S, 15S, 156S, 17S, 18S, 19S, 20S, 21S, 22S, 23S, 24S, 25S, 28S, or 29S. In some embodiments, the sense strand or the antisense strand comprises modification pattern ASO1.

In some embodiments, purines of the sense strand comprise 2′ fluoro modified purines. In some embodiments, purines of the sense strand comprise 2′-O-methyl modified purines. In some embodiments, purines of the sense strand comprise a mixture of 2′ fluoro and 2′-O-methyl modified purines. In some embodiments, all purines of the sense strand comprise 2′ fluoro modified purines. In some embodiments, all purines of the sense strand comprise 2′-O-methyl modified purines. In some embodiments, all purines of the sense strand comprise a mixture of 2′ fluoro and 2′-O-methyl modified purines.

In some embodiments, pyrimidines of the sense strand comprise 2′ fluoro modified pyrimidines. In some embodiments, pyrimidines of the sense strand comprise 2′-O-methyl modified pyrimidines. In some embodiments, pyrimidines of the sense strand comprise a mixture of 2′ fluoro and 2′-O-methyl modified pyrimidines. In some embodiments, all pyrimidines of the sense strand comprise 2′ fluoro modified pyrimidines. In some embodiments, all pyrimidines of the sense strand comprise 2′-O-methyl modified pyrimidines. In some embodiments, all pyrimidines of the sense strand comprise a mixture of 2′ fluoro and 2′-O-methyl modified pyrimidines.

In some embodiments, purines of the sense strand comprise 2′ fluoro modified purines, and pyrimidines of the sense strand comprise a mixture of 2′ fluoro and 2′-O-methyl modified pyrimidines. In some embodiments, purines of the sense strand comprise 2′-O-methyl modified purines, and pyrimidines of the sense strand comprise a mixture of 2′ fluoro and 2′-O-methyl modified pyrimidines. In some embodiments, purines of the sense strand comprise 2′ fluoro modified purines, and pyrimidines of the sense strand comprise 2′-O-methyl modified pyrimidines. In some embodiments, purines of the sense strand comprise 2′-O-methyl modified purines, and pyrimidines of the sense strand comprise 2′ fluoro modified pyrimidines. In some embodiments, pyrimidines of the sense strand comprise 2′ fluoro modified pyrimidines, and purines of the sense strand comprise a mixture of 2′ fluoro and 2′-O-methyl modified purines. In some embodiments, pyrimidines of the sense strand comprise 2′-O-methyl modified pyrimidines, and purines of the sense strand comprise a mixture of 2′ fluoro and 2′-O-methyl modified purines. In some embodiments, pyrimidines of the sense strand comprise 2′ fluoro modified pyrimidines, and purines of the sense strand comprise 2′-O-methyl modified purines. In some embodiments, pyrimidines of the sense strand comprise 2′-O-methyl modified pyrimidines, and purines of the sense strand comprise 2′ fluoro modified purines.

In some embodiments, all purines of the sense strand comprise 2′ fluoro modified purines, and all pyrimidines of the sense strand comprise a mixture of 2′ fluoro and 2′-O-methyl modified pyrimidines. In some embodiments, all purines of the sense strand comprise 2′-O-methyl modified purines, and all pyrimidines of the sense strand comprise a mixture of 2′ fluoro and 2′-O-methyl modified pyrimidines. In some embodiments, all purines of the sense strand comprise 2′ fluoro modified purines, and all pyrimidines of the sense strand comprise 2′-O-methyl modified pyrimidines. In some embodiments, all purines of the sense strand comprise 2′-O-methyl modified purines, and all pyrimidines of the sense strand comprise 2′ fluoro modified pyrimidines. In some embodiments, all pyrimidines of the sense strand comprise 2′ fluoro modified pyrimidines, and all purines of the sense strand comprise a mixture of 2′ fluoro and 2′-O-methyl modified purines. In some embodiments, all pyrimidines of the sense strand comprise 2′-O-methyl modified pyrimidines, and all purines of the sense strand comprise a mixture of 2′ fluoro and 2′-O-methyl modified purines. In some embodiments, all pyrimidines of the sense strand comprise 2′ fluoro modified pyrimidines, and all purines of the sense strand comprise 2′-O-methyl modified purines. In some embodiments, all pyrimidines of the sense strand comprise 2′-O-methyl modified pyrimidines, and all purines of the sense strand comprise 2′ fluoro modified purines.

In some embodiments, purines of the antisense strand comprise 2′ fluoro modified purines. In some embodiments, purines of the antisense strand comprise 2′-O-methyl modified purines. In some embodiments, purines of the antisense strand comprise a mixture of 2′ fluoro and 2′-O-methyl modified purines. In some embodiments, all purines of the antisense strand comprise 2′ fluoro modified purines. In some embodiments, all purines of the antisense strand comprise 2′-O-methyl modified purines. In some embodiments, all purines of the antisense strand comprise a mixture of 2′ fluoro and 2′-O-methyl modified purines.

In some embodiments, pyrimidines of the antisense strand comprise 2′ fluoro modified pyrimidines. In some embodiments, pyrimidines of the antisense strand comprise 2′-O-methyl modified pyrimidines. In some embodiments, pyrimidines of the antisense strand comprise a mixture of 2′ fluoro and 2′-O-methyl modified pyrimidines. In some embodiments, all pyrimidines of the antisense strand comprise 2′ fluoro modified pyrimidines. In some embodiments, all pyrimidines of the antisense strand comprise 2′-O-methyl modified pyrimidines. In some embodiments, all pyrimidines of the antisense strand comprise a mixture of 2′ fluoro and 2′-O-methyl modified pyrimidines.

In some embodiments, purines of the antisense strand comprise 2′ fluoro modified purines, and pyrimidines of the antisense strand comprise a mixture of 2′ fluoro and 2′-O-methyl modified pyrimidines. In some embodiments, purines of the antisense strand comprise 2′-O-methyl modified purines, and pyrimidines of the antisense strand comprise a mixture of 2′ fluoro and 2′-O-methyl modified pyrimidines. In some embodiments, purines of the antisense strand comprise 2′ fluoro modified purines, and pyrimidines of the antisense strand comprise 2′-O-methyl modified pyrimidines. In some embodiments, purines of the antisense strand comprise 2′-O-methyl modified purines, and pyrimidines of the antisense strand comprise 2′ fluoro modified pyrimidines. In some embodiments, pyrimidines of the antisense strand comprise 2′ fluoro modified pyrimidines, and purines of the antisense strand comprise a mixture of 2′ fluoro and 2′-O-methyl modified purines. In some embodiments, pyrimidines of the antisense strand comprise 2′-O-methyl modified pyrimidines, and purines of the antisense strand comprise a mixture of 2′ fluoro and 2′-O-methyl modified purines. In some embodiments, pyrimidines of the antisense strand comprise 2′ fluoro modified pyrimidines, and purines of the antisense strand comprise 2′-O-methyl modified purines. In some embodiments, pyrimidines of the antisense strand comprise 2′-O-methyl modified pyrimidines, and purines of the antisense strand comprise 2′ fluoro modified purines.

In some embodiments, all purines of the antisense strand comprise 2′ fluoro modified purines, and all pyrimidines of the antisense strand comprise a mixture of 2′ fluoro and 2′-O-methyl modified pyrimidines. In some embodiments, all purines of the antisense strand comprise 2′-O-methyl modified purines, and all pyrimidines of the antisense strand comprise a mixture of 2′ fluoro and 2′-O-methyl modified pyrimidines. In some embodiments, all purines of the antisense strand comprise 2′ fluoro modified purines, and all pyrimidines of the antisense strand comprise 2′-O-methyl modified pyrimidines. In some embodiments, all purines of the antisense strand comprise 2′-O-methyl modified purines, and all pyrimidines of the antisense strand comprise 2′ fluoro modified pyrimidines. In some embodiments, all pyrimidines of the antisense strand comprise 2′ fluoro modified pyrimidines, and all purines of the antisense strand comprise a mixture of 2′ fluoro and 2′-O-methyl modified purines. In some embodiments, all pyrimidines of the antisense strand comprise 2′-O-methyl modified pyrimidines, and all purines of the antisense strand comprise a mixture of 2′ fluoro and 2′-O-methyl modified purines. In some embodiments, all pyrimidines of the antisense strand comprise 2′ fluoro modified pyrimidines, and all purines of the antisense strand comprise 2′-O-methyl modified purines. In some embodiments, all pyrimidines of the antisense strand comprise 2′-O-methyl modified pyrimidines, and all purines of the antisense strand comprise 2′ fluoro modified purines.

Disclosed herein, in some embodiments, are modified oligonucleotides. The modified oligonucleotide may be an siRNA that includes modifications to the ribose rings, and phosphate linkages. The modifications may be in particular patterns that maximize cell delivery, stability, and efficiency. The siRNA may also include a vinyl phosphonate and a hydrophobic group. These modifications may aid in delivery to a cell or tissue within a subject. The modified oligonucleotide may be used in a method such as a treatment method or a method of reducing gene expression.

In some embodiments, the oligonucleotide comprises a duplex consisting of 21 nucleotide single strands with base pairing between 19 of the base pairs. In some embodiments, the duplex comprises single-stranded 2 nucleotide overhangs are at the 3′ ends of each strand. One strand (antisense strand) is complementary to a FGG mRNA. Each end of the antisense strand has one to two phosphorothioate bonds. The 5′ end has an optional phosphate mimic such as a vinyl phosphonate. In some embodiments, the oligonucleotide is used to knock down a FGG mRNA or a target protein. In some embodiments, the sense strand has the same sequence as the FGG mRNA. In some embodiments, there are 1-2 phosphorothioates at the 3′ end. In some embodiments, there are 1 or no phosphorothioates at the 5′ end. In some embodiments, there is a hydrophobic conjugate of 12 to 25 carbons attached at the 5′ end via a phosphodiester bond.

In some cases, the sense strand of any of the siRNAs comprises siRNA with a particular modification pattern. In some embodiments of the modification pattern, position 9 counting from the 5′ end of the sense strand may have a 2′F modification. In some embodiments, when position 9 of the sense strand is a pyrimidine, then all purines in the sense strand have a 2′OMe modification. In some embodiments, when position 9 is the only pyrimidine between positions 5 and 11 of the sense stand, then position 9 is the only position with a 2′F modification in the sense strand. In some embodiments, when position 9 and only one other base between positions 5 and 11 of the sense strand are pyrimidines, then both of these pyrimidines are the only two positions with a 2′F modification in the sense strand. In some embodiments, when position 9 and only two other bases between positions 5 and 11 of the sense strand are pyrimidines, and those two other pyrimidines are in adjacent positions so that there would be not three 2′F modifications in a row, then any combination of 2′F modifications can be made that give three 2′F modifications in total. In some embodiments, when there are more than 2 pyrimidines between positions 5 and 11 of the sense strand, then all combinations of pyrimidines having the 2′F modification are allowed that have three to five 2′F modifications in total, provided that the sense strand does not have three 2′F modifications in a row. In some cases, the sense strand of any of the siRNAs comprises a modification pattern which conforms to any or all of these sense strand rules.

In some embodiments, when position 9 of the sense strand is a purine, then all purines in the sense strand have a 2′OMe modification. In some embodiments, when position 9 is the only purine between positions 5 and 11 of the sense stand, then position 9 is the only position with a 2′F modification in the sense strand. In some embodiments, when position 9 and only one other base between positions 5 and 11 of the sense strand are purines, then both of these purines are the only two positions with a 2′F modification in the sense strand. In some embodiments, when position 9 and only two other bases between positions 5 and 11 of the sense strand are purines, and those two other purines are in adjacent positions so that there would be not three 2′F modifications in a row, then any combination of 2′F modifications can be made that give three 2′F modifications in total. In some embodiments, when there are more than 2 purines between positions 5 and 11 of the sense strand, then all combinations of purines having the 2′F modification are allowed that have three to five 2′F modifications in total, provided that the sense strand does not have three 2′F modifications in a row. In some cases, the sense strand of any of the siRNAs comprises a modification pattern which conforms to any or all of these sense strand rules.

In some cases, position 9 of the sense strand can be a 2′deoxy. In these cases, 2′F and 2′OMe modifications may occur at the other positions of the sense strand. In some cases, the sense strand of any of the siRNAs comprises a modification pattern which conforms to these sense strand rules.

In some cases, the sense strand of any of the siRNAs comprises a modification pattern which conforms to these sense strand rules.

Disclosed herein, in some embodiments are compositions comprising an oligonucleotide that targets FGG and when administered to a cell decreases expression of FGG, wherein the oligonucleotide comprises a small interfering RNA (siRNA) comprising a sense strand and an antisense strand, wherein the sense strand comprises a sense strand sequence described herein in which at least one internucleoside linkage is modified and at least one nucleoside is modified, or an sense strand sequence comprising 1 or 2 nucleoside substitutions, additions, or deletions of the oligonucleotide sequence in which at least one internucleoside linkage is modified and at least one nucleoside is modified, and wherein the antisense strand comprises an antisense strand sequence described herein in which at least one internucleoside linkage is modified and at least one nucleoside is modified, or an oligonucleotide sequence comprising 1 or 2 nucleoside substitutions, additions, or deletions of the antisense strand sequence in which at least one internucleoside linkage is modified and at least one nucleoside is modified. Some embodiments relate to methods that include administering the composition to a subject.

In some embodiments, the sense and/or antisense strand comprises a nucleoside sequence at least 70% identical, at least 75% identical, at least 80% identical, at least 85% identical, at least 90% identical, or at least 95% identical, to a sense and/or antisense strand sequence in any of Tables 8-15, 18A, 22A, 26A, 31A, 33A, 37A, 42A, 66A or 81. In some embodiments, the siRNA comprises the sense strand and/or the antisense strand sequence of an siRNA in any of Tables 8-15, 18A, 22A, 26A, 31A, 33A, 37A, 42A, 66A or 81 or a nucleic acid sequence thereof having 3 or 4 nucleoside substitutions, additions, or deletions. In some embodiments, the siRNA comprises the sense strand and/or the antisense strand sequence of an siRNA in any of Tables 8-15, 18A, 22A, 26A, 31A, 33A, 37A, 42A, 66A or 81 or a nucleic acid sequence thereof having 1 or 2 nucleoside substitutions, additions, or deletions. In some embodiments, the siRNA comprises the sense strand and/or the antisense strand sequence of an siRNA in any of Tables 8-15, 18A, 22A, 26A, 31A, 33A, 37A, 42A, 66A or 81. The siRNA may include the same internucleoside linkage modifications or nucleoside modifications as those in any of Tables 8-15, 18A, 22A, 26A, 31A, 33A, 37A, 42A, 66A or 81. The siRNA may include any different internucleoside linkage modifications or nucleoside modifications different from those in any of Tables 8-15, 18A, 22A, 26A, 31A, 33A, 37A, 42A, 66A or 81. The siRNA may include some unmodified internucleoside linkages or nucleosides.

In some embodiments, the sense and/or antisense strand comprises a nucleoside sequence at least 70% identical, at least 75% identical, at least 80% identical, at least 85% identical, at least 90% identical, or at least 95% identical, to a sense and/or antisense strand sequence in Table 8A. In some embodiments, the siRNA comprises the sense strand and/or the antisense strand sequence of an siRNA in Table 8A or a nucleic acid sequence thereof having 3 or 4 nucleoside substitutions, additions, or deletions. In some embodiments, the siRNA comprises the sense strand and/or the antisense strand sequence of an siRNA in Table 8A or a nucleic acid sequence thereof having 1 or 2 nucleoside substitutions, additions, or deletions. In some embodiments, the siRNA comprises the sense strand and/or the antisense strand sequence of an siRNA in Table 8A. The siRNA may include the same internucleoside linkage modifications or nucleoside modifications as those in Table 8A. The siRNA may include any different internucleoside linkage modifications or nucleoside modifications different from those in Table 8A. The siRNA may include some unmodified internucleoside linkages or nucleosides.

In some embodiments, the sense and/or antisense strand comprises a nucleoside sequence at least 70% identical, at least 75% identical, at least 80% identical, at least 85% identical, at least 90% identical, or at least 95% identical, to a sense and/or antisense strand sequence in Table 8B. In some embodiments, the siRNA comprises the sense strand and/or the antisense strand sequence of an siRNA in Table 8B or a nucleic acid sequence thereof having 3 or 4 nucleoside substitutions, additions, or deletions. In some embodiments, the siRNA comprises the sense strand and/or the antisense strand sequence of an siRNA in Table 8B or a nucleic acid sequence thereof having 1 or 2 nucleoside substitutions, additions, or deletions. In some embodiments, the siRNA comprises the sense strand and/or the antisense strand sequence of an siRNA in Table 8B. The siRNA may include the same internucleoside linkage modifications or nucleoside modifications as those in Table 8B. The siRNA may include any different internucleoside linkage modifications or nucleoside modifications different from those in Table 8B. The siRNA may include some unmodified internucleoside linkages or nucleosides.

In some embodiments, the sense and/or antisense strand comprises a nucleoside sequence at least 70% identical, at least 75% identical, at least 80% identical, at least 85% identical, at least 90% identical, or at least 95% identical, to a sense and/or antisense strand sequence in Table 81. In some embodiments, the siRNA comprises the sense strand and/or the antisense strand sequence of an siRNA in Table 81 or a nucleic acid sequence thereof having 3 or 4 nucleoside substitutions, additions, or deletions. In some embodiments, the siRNA comprises the sense strand and/or the antisense strand sequence of an siRNA in Table 81 or a nucleic acid sequence thereof having 1 or 2 nucleoside substitutions, additions, or deletions. In some embodiments, the siRNA comprises the sense strand and/or the antisense strand sequence of an siRNA in Table 81. The siRNA may include the same internucleoside linkage modifications or nucleoside modifications as those in Table 81. The siRNA may include any different internucleoside linkage modifications or nucleoside modifications different from those in Table 81. The siRNA may include some unmodified internucleoside linkages or nucleosides.

In some embodiments, the sense strand comprises a nucleoside sequence at least 70% identical, at least 75% identical, at least 80% identical, at least 85% identical, at least 90% identical, or at least 95% identical, to any one of SEQ ID NOs: 3591-3594. In some embodiments, the sense strand comprises the nucleoside sequence of any one of SEQ ID NOS: 3591-3594, or a sense strand sequence thereof having 1 or 2 nucleoside substitutions, additions, or deletions. In some embodiments, the sense strand comprises the nucleoside sequence of any one of SEQ ID NOS: 3591-3594, and 3 or 4 nucleoside substitutions, additions, or deletions. In some embodiments, the sense strand comprises the nucleoside sequence of any one of SEQ ID NOS: 3591-3594. The sense strand may include any different internucleoside linkage modifications or nucleoside modifications different from those in SEQ ID NOS: 3591-3594. The sense strand may include some unmodified internucleoside linkages or nucleosides. The sense strand may include GalNAc1 or another GalNAc moiety.

In some embodiments, the sense strand comprises a nucleoside sequence at least 85% identical to SEQ ID NO: 3591. In some embodiments, the sense strand comprises the nucleoside sequence of SEQ ID NO: 3591, or a sense strand sequence thereof having 1 or 2 nucleoside substitutions, additions, or deletions. In some embodiments, the sense strand comprises the nucleoside sequence of SEQ ID NO: 3591, and 3 or 4 nucleoside substitutions, additions, or deletions. In some embodiments, the sense strand comprises the nucleoside sequence of SEQ ID NO: 3591. The sense strand may include any different internucleoside linkage modifications or nucleoside modifications different from those in SEQ ID NO: 3591. The sense strand may include some unmodified internucleoside linkages or nucleosides. The sense strand may include GalNAc1 or another GalNAc moiety.

In some embodiments, the sense strand comprises a nucleoside sequence at least 85% identical to SEQ ID NO: 3592. In some embodiments, the sense strand comprises the nucleoside sequence of SEQ ID NO: 3592, or a sense strand sequence thereof having 1 or 2 nucleoside substitutions, additions, or deletions. In some embodiments, the sense strand comprises the nucleoside sequence of SEQ ID NO: 3592, and 3 or 4 nucleoside substitutions, additions, or deletions. In some embodiments, the sense strand comprises the nucleoside sequence of SEQ ID NO: 3592. The sense strand may include any different internucleoside linkage modifications or nucleoside modifications different from those in SEQ ID NO: 3592. The sense strand may include some unmodified internucleoside linkages or nucleosides. The sense strand may include GalNAc1 or another GalNAc moiety.

In some embodiments, the sense strand comprises a nucleoside sequence at least 85% identical to SEQ ID NO: 3593. In some embodiments, the sense strand comprises the nucleoside sequence of SEQ ID NO: 3593, or a sense strand sequence thereof having 1 or 2 nucleoside substitutions, additions, or deletions. In some embodiments, the sense strand comprises the nucleoside sequence of SEQ ID NO: 3593, and 3 or 4 nucleoside substitutions, additions, or deletions. In some embodiments, the sense strand comprises the nucleoside sequence of SEQ ID NO: 3593. The sense strand may include any different internucleoside linkage modifications or nucleoside modifications different from those in SEQ ID NO: 3593. The sense strand may include some unmodified internucleoside linkages or nucleosides. The sense strand may include GalNAc1 or another GalNAc moiety.

In some embodiments, the sense strand comprises a nucleoside sequence at least 85% identical to SEQ ID NO: 3594. In some embodiments, the sense strand comprises the nucleoside sequence of SEQ ID NO: 3594, or a sense strand sequence thereof having 1 or 2 nucleoside substitutions, additions, or deletions. In some embodiments, the sense strand comprises the nucleoside sequence of SEQ ID NO: 3594, and 3 or 4 nucleoside substitutions, additions, or deletions. In some embodiments, the sense strand comprises the nucleoside sequence of SEQ ID NO: 3594. The sense strand may include any different internucleoside linkage modifications or nucleoside modifications different from those in SEQ ID NO: 3594. The sense strand may include some unmodified internucleoside linkages or nucleosides. The sense strand may include GalNAc1 or another GalNAc moiety.

In some embodiments, the sense strand comprises a nucleoside sequence at least 70% identical, at least 75% identical, at least 80% identical, at least 85% identical, at least 90% identical, or at least 95% identical, to any one of SEQ ID NOs: 3641-3676. In some embodiments, the sense strand comprises the nucleoside sequence of any one of SEQ ID NOS: 3641-3676, or a sense strand sequence thereof having 1 or 2 nucleoside substitutions, additions, or deletions. In some embodiments, the sense strand comprises the nucleoside sequence of any one of SEQ ID NOS: 3641-3676, and 3 or 4 nucleoside substitutions, additions, or deletions. In some embodiments, the sense strand comprises the nucleoside sequence of any one of SEQ ID NOS: 3641-3676. The sense strand may include any different internucleoside linkage modifications or nucleoside modifications different from those in SEQ ID NOS: 3641-3676. The sense strand may include some unmodified internucleoside linkages or nucleosides. The sense strand may include GalNAc1 or another GalNAc moiety.

In some embodiments, the sense strand comprises a nucleoside sequence at least 85% identical to SEQ ID NO: 3651. In some embodiments, the sense strand comprises the nucleoside sequence of SEQ ID NO: 3651, or a sense strand sequence thereof having 1 or 2 nucleoside substitutions, additions, or deletions. In some embodiments, the sense strand comprises the nucleoside sequence of SEQ ID NO: 3651, and 3 or 4 nucleoside substitutions, additions, or deletions. In some embodiments, the sense strand comprises the nucleoside sequence of SEQ ID NO: 3651. The sense strand may include any different internucleoside linkage modifications or nucleoside modifications different from those in SEQ ID NO: 3651. The sense strand may include some unmodified internucleoside linkages or nucleosides. The sense strand may include GalNAc1 or another GalNAc moiety.

In some embodiments, the sense strand comprises a nucleoside sequence at least 85% identical to SEQ ID NO: 3652. In some embodiments, the sense strand comprises the nucleoside sequence of SEQ ID NO: 3652, or a sense strand sequence thereof having 1 or 2 nucleoside substitutions, additions, or deletions. In some embodiments, the sense strand comprises the nucleoside sequence of SEQ ID NO: 3652, and 3 or 4 nucleoside substitutions, additions, or deletions. In some embodiments, the sense strand comprises the nucleoside sequence of SEQ ID NO: 3652. The sense strand may include any different internucleoside linkage modifications or nucleoside modifications different from those in SEQ ID NO: 3652. The sense strand may include some unmodified internucleoside linkages or nucleosides. The sense strand may include GalNAc1 or another GalNAc moiety.

In some embodiments, the sense strand comprises a nucleoside sequence at least 85% identical to SEQ ID NO: 3654. In some embodiments, the sense strand comprises the nucleoside sequence of SEQ ID NO: 3654, or a sense strand sequence thereof having 1 or 2 nucleoside substitutions, additions, or deletions. In some embodiments, the sense strand comprises the nucleoside sequence of SEQ ID NO: 3654, and 3 or 4 nucleoside substitutions, additions, or deletions. In some embodiments, the sense strand comprises the nucleoside sequence of SEQ ID NO: 3654. The sense strand may include any different internucleoside linkage modifications or nucleoside modifications different from those in SEQ ID NO: 3594. The sense strand may include some unmodified internucleoside linkages or nucleosides. The sense strand may include GalNAc1 or another GalNAc moiety.

In some embodiments, the sense strand comprises a nucleoside sequence at least 85% identical to SEQ ID NO: 3675. In some embodiments, the sense strand comprises the nucleoside sequence of SEQ ID NO: 3675, or a sense strand sequence thereof having 1 or 2 nucleoside substitutions, additions, or deletions. In some embodiments, the sense strand comprises the nucleoside sequence of SEQ ID NO: 3675, and 3 or 4 nucleoside substitutions, additions, or deletions. In some embodiments, the sense strand comprises the nucleoside sequence of SEQ ID NO: 3675. The sense strand may include any different internucleoside linkage modifications or nucleoside modifications different from those in SEQ ID NO: 3675. The sense strand may include some unmodified internucleoside linkages or nucleosides. The sense strand may include GalNAc1 or another GalNAc moiety.

In some embodiments, the antisense strand comprises a nucleoside sequence at least 70% identical, at least 75% identical, at least 80% identical, at least 85% identical, at least 90% identical, or at least 95% identical, to any one of SEQ ID NOs: 3595-3598. In some embodiments, the antisense strand comprises the nucleoside sequence of any one of SEQ ID NOS: 3595-3598, or an antisense strand sequence thereof having 1 or 2 nucleoside substitutions, additions, or deletions. In some embodiments, the antisense strand comprises the nucleoside sequence of any one of SEQ ID NOS: 3595-3598, and 3 or 4 nucleoside substitutions, additions, or deletions. In some embodiments, the antisense strand comprises the nucleoside sequence of any one of SEQ ID NOS: 3595-3598. The antisense strand may include any different internucleoside linkage modifications or nucleoside modifications different from those in SEQ ID NOS: 3595-3598. The antisense strand may include some unmodified internucleoside linkages or nucleosides. The antisense strand may include a GalNAc moiety.

In some embodiments, the antisense strand comprises a nucleoside sequence at least 85% identical to SEQ ID NO: 3595. In some embodiments, the antisense strand comprises the nucleoside sequence of SEQ ID NO: 3595, or an antisense strand sequence thereof having 1 or 2 nucleoside substitutions, additions, or deletions. In some embodiments, the antisense strand comprises the nucleoside sequence of SEQ ID NO: 3595, and 3 or 4 nucleoside substitutions, additions, or deletions. In some embodiments, the antisense strand comprises the nucleoside sequence of SEQ ID NO: 3595. The antisense strand may include any different internucleoside linkage modifications or nucleoside modifications different from those in SEQ ID NO: 3595. The antisense strand may include some unmodified internucleoside linkages or nucleosides. The antisense strand may include a GalNAc moiety.

In some embodiments, the antisense strand comprises a nucleoside sequence at least 85% identical to SEQ ID NO: 3596. In some embodiments, the antisense strand comprises the nucleoside sequence of SEQ ID NO: 3596, or an antisense strand sequence thereof having 1 or 2 nucleoside substitutions, additions, or deletions. In some embodiments, the antisense strand comprises the nucleoside sequence of SEQ ID NO: 3596, and 3 or 4 nucleoside substitutions, additions, or deletions. In some embodiments, the antisense strand comprises the nucleoside sequence of SEQ ID NO: 3596. The antisense strand may include any different internucleoside linkage modifications or nucleoside modifications different from those in SEQ ID NO: 3596. The antisense strand may include some unmodified internucleoside linkages or nucleosides. The antisense strand may include a GalNAc moiety.

In some embodiments, the antisense strand comprises a nucleoside sequence at least 85% identical to SEQ ID NO: 3597. In some embodiments, the antisense strand comprises the nucleoside sequence of SEQ ID NO: 3597, or an antisense strand sequence thereof having 1 or 2 nucleoside substitutions, additions, or deletions. In some embodiments, the antisense strand comprises the nucleoside sequence of SEQ ID NO: 3597, and 3 or 4 nucleoside substitutions, additions, or deletions. In some embodiments, the antisense strand comprises the nucleoside sequence of SEQ ID NO: 3597. The antisense strand may include any different internucleoside linkage modifications or nucleoside modifications different from those in SEQ ID NO: 3597. The antisense strand may include some unmodified internucleoside linkages or nucleosides. The antisense strand may include a GalNAc moiety.

In some embodiments, the antisense strand comprises a nucleoside sequence at least 85% identical to SEQ ID NO: 3598. In some embodiments, the antisense strand comprises the nucleoside sequence of SEQ ID NO: 3598, or an antisense strand sequence thereof having 1 or 2 nucleoside substitutions, additions, or deletions. In some embodiments, the antisense strand comprises the nucleoside sequence of SEQ ID NO: 3598, and 3 or 4 nucleoside substitutions, additions, or deletions. In some embodiments, the antisense strand comprises the nucleoside sequence of SEQ ID NO: 3598. The antisense strand may include any different internucleoside linkage modifications or nucleoside modifications different from those in SEQ ID NO: 3598. The antisense strand may include some unmodified internucleoside linkages or nucleosides. The antisense strand may include a GalNAc moiety.

In some embodiments, the antisense strand comprises a nucleoside sequence at least 70% identical, at least 75% identical, at least 80% identical, at least 85% identical, at least 90% identical, or at least 95% identical, to any one of SEQ ID NOs: 3677-3712. In some embodiments, the antisense strand comprises the nucleoside sequence of any one of SEQ ID NOS: 3677-3712, or an antisense strand sequence thereof having 1 or 2 nucleoside substitutions, additions, or deletions. In some embodiments, the antisense strand comprises the nucleoside sequence of any one of SEQ ID NOS: 3677-3712, and 3 or 4 nucleoside substitutions, additions, or deletions. In some embodiments, the antisense strand comprises the nucleoside sequence of any one of SEQ ID NOS: 3677-3712. The antisense strand may include any different internucleoside linkage modifications or nucleoside modifications different from those in SEQ ID NOS: 3677-3712. The antisense strand may include some unmodified internucleoside linkages or nucleosides. The antisense strand may include a GalNAc moiety.

In some embodiments, the antisense strand comprises a nucleoside sequence at least 85% identical to SEQ ID NO: 3687. In some embodiments, the antisense strand comprises the nucleoside sequence of SEQ ID NO: 3687, or an antisense strand sequence thereof having 1 or 2 nucleoside substitutions, additions, or deletions. In some embodiments, the antisense strand comprises the nucleoside sequence of SEQ ID NO: 3687, and 3 or 4 nucleoside substitutions, additions, or deletions. In some embodiments, the antisense strand comprises the nucleoside sequence of SEQ ID NO: 3687. The antisense strand may include any different internucleoside linkage modifications or nucleoside modifications different from those in SEQ ID NO: 3687. The antisense strand may include some unmodified internucleoside linkages or nucleosides. The antisense strand may include a GalNAc moiety.

In some embodiments, the antisense strand comprises a nucleoside sequence at least 85% identical to SEQ ID NO: 3688. In some embodiments, the antisense strand comprises the nucleoside sequence of SEQ ID NO: 3688, or an antisense strand sequence thereof having 1 or 2 nucleoside substitutions, additions, or deletions. In some embodiments, the antisense strand comprises the nucleoside sequence of SEQ ID NO: 3688, and 3 or 4 nucleoside substitutions, additions, or deletions. In some embodiments, the antisense strand comprises the nucleoside sequence of SEQ ID NO: 3688. The antisense strand may include any different internucleoside linkage modifications or nucleoside modifications different from those in SEQ ID NO: 3688. The antisense strand may include some unmodified internucleoside linkages or nucleosides. The antisense strand may include a GalNAc moiety.

In some embodiments, the antisense strand comprises a nucleoside sequence at least 85% identical to SEQ ID NO: 3690. In some embodiments, the antisense strand comprises the nucleoside sequence of SEQ ID NO: 3690, or an antisense strand sequence thereof having 1 or 2 nucleoside substitutions, additions, or deletions. In some embodiments, the antisense strand comprises the nucleoside sequence of SEQ ID NO: 3690, and 3 or 4 nucleoside substitutions, additions, or deletions. In some embodiments, the antisense strand comprises the nucleoside sequence of SEQ ID NO: 3690. The antisense strand may include any different internucleoside linkage modifications or nucleoside modifications different from those in SEQ ID NO: 3690. The antisense strand may include some unmodified internucleoside linkages or nucleosides. The antisense strand may include a GalNAc moiety.

In some embodiments, the antisense strand comprises a nucleoside sequence at least 85% identical to SEQ ID NO: 3747. In some embodiments, the antisense strand comprises the nucleoside sequence of SEQ ID NO: 3747, or an antisense strand sequence thereof having 1 or 2 nucleoside substitutions, additions, or deletions. In some embodiments, the antisense strand comprises the nucleoside sequence of SEQ ID NO: 3747, and 3 or 4 nucleoside substitutions, additions, or deletions. In some embodiments, the antisense strand comprises the nucleoside sequence of SEQ ID NO: 3747. The antisense strand may include any different internucleoside linkage modifications or nucleoside modifications different from those in SEQ ID NO: 3747. The antisense strand may include some unmodified internucleoside linkages or nucleosides. The antisense strand may include a GalNAc moiety.

### 4. ASO Modification Patterns

In some embodiments, the composition comprises an oligonucleotide that inhibits the expression of FGG, wherein the oligonucleotide comprises an antisense oligonucleotide (ASO). In some embodiments, the ASO comprises modification pattern ASO1: 5′-nsnsnsnsnsdNsdNsdNsdNsdNsdNsdNsdNsdNsdNsnsnsnsnsn-3′ (SEQ ID NO: 3640), wherein “dN” is any deoxynucleotide, “n” is a 2′O-methyl or 2′O-methoxyethyl-modified nucleoside, and “s” is a phosphorothioate linkage. In some embodiments, the ASO comprises modification pattern 1S, 2S, 3S, 4S, 5S, 6S, 7S, 8S, 9S, 10S, 11S, 12S, 13S, 14S, 15S, 156S, 17S, 18S, 19S, 20S, 21S, 22S, 23S, 24S, 25S, 28S, 29S, 1AS, 2AS, 3AS, 4AS, 5AS, 6AS, 7AS, 8AS, 9AS, 10AS, 11AS, 12AS, 13AS, 14AS, 15AS, 16AS, 17AS, 18AS, 19AS, 20AS, or 21AS.

### D. Formulations

In some embodiments, the composition is a pharmaceutical composition. In some embodiments, the composition is sterile. In some embodiments, the composition further comprises a pharmaceutically acceptable carrier.

In some embodiments, the pharmaceutically acceptable carrier comprises water. In some embodiments, the pharmaceutically acceptable carrier comprises a buffer. In some embodiments, the pharmaceutically acceptable carrier comprises a saline solution. In some embodiments, the pharmaceutically acceptable carrier comprises water, a buffer, or a saline solution. In some embodiments, the composition comprises a liposome. In some embodiments, the pharmaceutically acceptable carrier comprises liposomes, lipids, nanoparticles, proteins, protein-antibody complexes, peptides, cellulose, nanogel, or a combination thereof.

### II. Methods and Uses

Disclosed herein, in some embodiments, are methods of administering a composition described herein to a subject. Some embodiments relate to use a composition described herein, such as administering the composition to a subject.

Some embodiments relate to a method of treating a disease or disorder (e.g., mental disorder (e.g., psychiatric disorder or neurological disorder)) in a subject in need thereof. Some embodiments relate to use of a composition described herein in the method of treatment. Some embodiments include administering a composition described herein to a subject with the disorder. In some embodiments, the administration treats the disorder in the subject. In some embodiments, the composition treats the disorder in the subject.

In some embodiments, the treatment comprises prevention, inhibition, or reversion of the disorder (e.g., mental disorder (e.g., psychiatric disorder or neurological disorder)) in the subject. Some embodiments relate to use of a composition described herein in the method of preventing, inhibiting, or reversing the disorder. Some embodiments relate to a method of preventing, inhibiting, or reversing a disorder in a subject in need thereof. Some embodiments include administering a composition described herein to a subject with the disorder. In some embodiments, the administration prevents, inhibits, or reverses the disorder in the subject. In some embodiments, the composition prevents, inhibits, or reverses the disorder in the subject.

Some embodiments relate to a method of preventing a disorder (e.g., mental disorder (e.g., psychiatric disorder or neurological disorder)) in a subject in need thereof. Some embodiments relate to use of a composition described herein in the method of preventing the disorder. Some embodiments include administering a composition described herein to a subject with the disorder. In some embodiments, the administration prevents the disorder in the subject. In some embodiments, the composition prevents the disorder in the subject.

Some embodiments relate to a method of inhibiting a disorder (e.g., mental disorder (e.g., psychiatric disorder or neurological disorder)) in a subject in need thereof. Some embodiments relate to use of a composition described herein in the method of inhibiting the disorder. Some embodiments include administering a composition described herein to a subject with the disorder. In some embodiments, the administration inhibits the disorder in the subject. In some embodiments, the composition inhibits the disorder in the subject.

Some embodiments relate to a method of reversing a disorder (e.g., mental disorder (e.g., psychiatric disorder or neurological disorder)) in a subject in need thereof. Some embodiments relate to use of a composition described herein in the method of reversing the disorder. Some embodiments include administering a composition described herein to a subject with the disorder. In some embodiments, the administration reverses the disorder in the subject. In some embodiments, the composition reverses the disorder in the subject.

In some embodiments, the administration is systemic. In some embodiments, the administration is intravenous. In some embodiments, the administration is by injection.

### A. Disorders

Some embodiments of the methods described herein include treating a disorder in a subject in need thereof. A disorder can include a disease. In some embodiments, the disorder is a mental disorder. In some embodiments, the mental disorder is a psychiatric disorder or neurological disorder. The psychiatric disorder or neurological disorder may comprise a hepatic disorder, a brain disorder, a CNS disorder, a CSF disorder, or a combination thereof.

In some embodiments, the disorder comprises a psychiatric disorder. Non-limiting examples of psychiatric disorders include depressive disorders, such as major depressive disorder, persistent depressive disorder, treatment resistant depression and signs or symptoms of depression. Further non-limiting examples of psychiatric disorders include post-traumatic stress disorder, mood disorders, anxiety disorders (e.g., generalized anxiety disorder, obsessive-compulsive disorder, panic disorder, social phobia, etc.), eating disorders, substance-use disorders (e.g., alcohol use disorders, prescription medicines use disorders, illegal drug use disorders, psychoactive substance-use disorders, etc.) bipolar disorder, personality disorders, schizophrenia and schizoaffective disorders.

In some embodiments, the disorder is a depressive disorder. Examples of depressive disorders include major depressive disorder, persistent depressive disorder, or treatment resistant depression. In some embodiments, the depressive disorder comprises or consists of major depressive disorder. In some embodiments, the depressive disorder comprises or consists of persistent depressive disorder. In some embodiments, the depressive disorder comprises or consists of treatment resistant depression. In some embodiments, the depressive disorder is treatment resistant depression. Treatment resistant depression may include depression that does not respond (e.g., within an acceptable period of time) to first, second, or third line treatments. In some embodiments, the disorder includes a sign or symptom of depression. Exemplary signs or symptoms of depression may include a persistent feeling of sadness or loss of interest, apathy, feelings of hopelessness and sadness, anxiety, agitation, and restlessness. Exemplary signs or symptoms of depression may be any sign or symptom of depression within the Diagnostic and Statistical Manual of Mental Disorders (DSM-5), which is hereby incorporated by reference.

In some embodiments, the disorder comprises post-traumatic stress disorder (PTSD). Exemplary signs or symptoms of PTSD include recurrent, unwanted distressing memories of the traumatic event, flashbacks, upsetting dreams or nightmares about the traumatic event, negative thoughts about yourself, other people or the world, memory problems, difficulty experiencing positive emotions, or feeling emotionally numb. Exemplary signs or symptoms of PTSD may be any sign or symptom of PTSD within the DSM-5.

In some embodiments, the disorder comprises mood disorders. An exemplary mood disorder includes dysthymia. In some embodiments, the disorder comprises anxiety disorders. Exemplary anxiety disorders include generalized anxiety disorder (GAD), obsessive-compulsive disorder (OCD), panic disorder, social phobias, and social anxiety disorder. Signs or symptoms of anxiety disorders include a feeling of restlessness, being easily fatigued, having difficulty concentration, and being irritable. Exemplary signs or symptoms of anxiety disorders (e.g., GAD, OCT, etc.) may be any sign or symptom of anxiety disorders within the DSM-5.

In some embodiments, the disorder comprises eating disorders. Exemplary eating disorders include anorexia nervosa, bulimia nervosa, and binge-eating disorder. Exemplary signs and symptoms of eating disorders include extremely restricted eating, emaciation, intense fear of gaining weight, brittle nails and hair, eating unusually large amounts of food in a specific amount of time, such as a 2-hour period, eating even when you're full or not hungry, and eating until you're uncomfortably full. Exemplary signs or symptoms of eating disorders may be any sign or symptom of eating disorders within the DSM-5.

In some embodiments, the disorder comprises substance-use disorders. Exemplary substance-use disorders include alcohol-use disorder, prescription drug use disorder, illegal drug use disorder, solvent abuse, and “legal high” abuse. Exemplary signs and symptoms of substance-use disorders include intense urges for the substance that block out other thoughts, needing more of the substance to get the same effect over time, and failure in attempts to stop using the substance. Exemplary signs or symptoms of substance-use disorders may be any sign or symptom of substance-use disorders within the DSM-5.

In some embodiments, the disorder comprises bipolar disorder. In some embodiments, the bipolar disorder comprises bipolar I disorder. In some embodiments, the bipolar disorder comprises bipolar II disorder. In some embodiments, the bipolar disorder comprises cyclothymic bipolar disorder. In some embodiments, the bipolar disorder comprises mixed feature bipolar disorder. Exemplary signs and symptoms of bipolar disorder include experiencing a manic episode and experiencing a major depressive episode. Exemplary signs or symptoms of bipolar disorder may be any sign or symptom of bipolar disorder within the DSM-5.

In some embodiments, the disorder comprises a personality disorder. Exemplary personality disorders include borderline personality disorder, antisocial personality disorder, histrionic personality disorder, narcissistic personality disorder, avoidant personality disorder, and schizoid personality disorder. Exemplary signs and symptoms of personality disorders include impulsive and risky behavior, unstable or fragile self-image, up and down moods, and suicidal behavior or threats of self-injury. Exemplary signs or symptoms of personality disorder may be any sign or symptom of personality disorder within the DSM-5.

In some embodiments, the disorder comprises schizophrenia. Exemplary schizophrenia signs and symptoms include delusions, hallucinations, disorganized thinking, and loss of interest or motivation in life. In some embodiments, the signs and symptoms comprise positive symptoms (e.g., hallucinations or delusions). In some embodiments, the signs and symptoms comprise negative symptoms (e.g., lack of interest or emotionally flat). Exemplary signs or symptoms of schizophrenia may be any sign or symptom of schizophrenia within the DSM-5.

In some embodiments, the disorder comprises schizoaffective disorders. Exemplary schizoaffective disorders include the bipolar type schizoaffective disorder and depressive type schizoaffective disorder. Exemplary signs and symptoms of schizoaffective disorders include delusions, hallucinations, impaired communication, and bizarre or unusual behavior. Exemplary signs or symptoms of schizoaffective disorders may be any sign or symptom of schizoaffective disorders within the DSM-5.

In some embodiments, the disorder comprises a neurological disorder. Non-limiting examples of neurological disorders include Alzheimer's disease, dementia, cognitive decline, vascular dementia. Further non-limiting examples of neurological disorders include headache, migraine (e.g., with aura and/or without aura), chronic pain, fibromyalgia, chronic fatigue syndrome (e.g. myalgic encephalomyelitis), motor neuron disease (e.g., Amyotrophic Lateral Sclerosis (ALS)).

In some embodiments, the disorder comprises dementia. In some embodiments, dementia comprises vascular dementia. In some embodiments, dementia comprises lewy body dementia. In some embodiments, dementia comprises frontotemporal dementia. In some embodiments, dementia comprises Alzheimer's disease. In some embodiments, dementia comprises mixed dementia. Exemplary signs and symptoms of dementia include memory loss, difficulty communicating, difficulty with visual and spatial abilities, difficulty reasoning or problem-solving, difficulty with coordination and motor functions, and confusion and disorientation.

In some embodiments, Alzheimer's disease comprises early-onset Alzheimer's disease. Early-onset Alzheimer's disease may occur in subjects under the age of 65 years old. In some embodiments, Alzheimer's disease comprises late-onset Alzheimer's disease. In some embodiments, Alzheimer's disease comprises common Alzheimer's disease. In some embodiments, Alzheimer's disease comprises genetic Alzheimer's disease. Exemplary signs and symptoms of Alzheimer's disease include increased memory loss and confusion, inability to learn new things, difficulty with language, difficulty organizing thoughts and thinking logically, shortened attention span, and problems coping with new situations.

In some embodiments, the disorder comprises delirium. Exemplary forms of delirium include hyperactive delirium, hypoactive delirium, and mixed delirium. Exemplary signs and symptoms of delirium include agitation, disorientation, delusional thoughts, hallucinations, poor memory, difficulty speaking and trouble understanding speech.

In some embodiments, the disorder comprises cognitive decline. Exemplary forms of cognitive decline include mild cognitive impairment, dementia, primary progressive aphasia, corticobasal degeneration, primary progressive aphasia, and progressive supranuclear palsy. Exemplary signs and symptoms of cognitive decline include forgetfulness, feelings of being overwhelmed, difficulty understanding directions or instructions, inability to organize tasks, and an increased impulsiveness.

In some embodiments, the disorder comprises a headache. In some embodiments, the headache comprises a migraine (e.g., with aura or without aura). Headaches may include sinus headaches, tension headache, migraine, and cluster headache. Exemplary signs and symptoms of headaches include pain (e.g., deep and constant) in the cheekbones, forehead, bridge of the nose, the cranium, or the back of the neck, aura, photophobia, phonophobia, and emesis.

In some embodiments, the disorder comprises chronic pain. In some embodiments, chronic pain comprises fibromyalgia. Exemplary signs and symptoms of fibromyalgia include muscular pain, fatigues, depression, anxiety, sleeplessness, headache, and difficulty concentrating. Exemplary chronic pain disorders include postsurgical pain, post-trauma pain, low back pain, cancer pain, arthritis pain, muscular pain, and neuropathic pain (e.g., diabetic neuropathy).

In some embodiments, the disorder comprises chronic fatigue syndrome (also referred to as myalgic encephalomyelitis). Exemplary signs and symptoms of chronic fatigue syndrome include extreme fatigue that lasts for extended periods of time (e.g., for at least six months) that cannot be fully explained by an underlying medical condition, fatigue that worsens with physical or mental activity, pain (e.g., joint or muscular), malaise, forgetfulness, anxiety, and depression.

In some embodiments, the disorder comprises a motor neuron disease. In some embodiments, the motor neuron disease is amyotrophic lateral sclerosis (ALS). Exemplary forms of motor neuron diseases include progressive bulbar palsy (PBP), progressive muscular atrophy (PMA), ALS, and primary lateral sclerosis (PLS). Exemplary signs and symptoms of motor neuron diseases (e.g., ALS) include motor control difficulties (e.g., difficulty walking or completing normal daily activities), muscular weakness, slurred speech, difficulty swallowing, muscle cramps and twitching (e.g., in the arms, shoulders, or tongue), and inappropriate crying, laughing or yawning,

In some embodiments, the disorder comprises a coagulation or clotting disorder. In some embodiments, the coagulation or clotting disorder comprises Hemophilia, Von Willebrand disease or clotting factor deficiencies. In some embodiments, the coagulation or clotting disorder comprises a thrombophilia. In some embodiments, the thrombophilia comprises an inherited thrombophilia. In some embodiments, the thrombophilia comprises an acquired thrombophilia. In some embodiments, the coagulation or clotting disorder comprises a hypercoagulable state. In some embodiments, the hypercoagulable state comprises cancer. In some embodiments, the hypercoagulable state comprises atrial fibrillation. In some embodiments, the hypercoagulable states comprises a post surgical period or immobility. In some embodiments, the coagulation or clotting disorder comprises arterial thrombosis or thromboembolism. In some embodiments, the coagulation or clotting disorder comprises venous thrombosis or thromboembolism. In some embodiments, the venous thromboembolism comprises deep venous thrombosis. In some embodiments, the venous thromboembolism comprises pulmonary embolism. In some embodiments, the venous thromboembolism comprises thrombophlebitis.

In some cases, the disorder may be diagnosed with the use of a questionnaire or a scoring system. In some cases, the disorder is diagnosed according to DSM-5 criteria. In some cases, the disorder is diagnosed by a healthcare professional (e.g., physician or the like).

In some embodiments, the disorder comprises one or more disorders (e.g., any of the disorders disclosed herein). In some embodiments, the disorder comprises two disorders. In some embodiments, the disorder comprises three disorders. In some embodiments, the disorder comprises four disorders. In some embodiments, the disorder comprises five disorders.

### B. Subjects

Some embodiments of the methods described herein include treatment of a subject. Non-limiting examples of subjects include vertebrates, animals, mammals, dogs, cats, cattle, rodents, mice, rats, primates, monkeys, and humans. In some embodiments, the subject is a vertebrate. In some embodiments, the subject is an animal. In some embodiments, the subject is a mammal. In some embodiments, the subject is a dog. In some embodiments, the subject is a cat. In some embodiments, the subject is a cattle. In some embodiments, the subject is a mouse. In some embodiments, the subject is a rat. In some embodiments, the subject is a primate. In some embodiments, the subject is a monkey. In some embodiments, the subject is an animal, a mammal, a dog, a cat, cattle, a rodent, a mouse, a rat, a primate, or a monkey. In some embodiments, the subject is a human.

In some embodiments, the subject is male. In some embodiments, the subject is female. In some embodiments, the subject is an adult (e.g. at least 18 years old). In some embodiments, the subject is 45 years old or greater. In some embodiments, the subject is 50 years old or greater. In some embodiments, the subject is 55 years old or greater. In some embodiments, the subject is 60 years old or greater. In some embodiments, the subject is 65 years old or greater. In some embodiments, the subject is 70 years old or greater. In some embodiments, the subject is 75 years old or greater. In some embodiments, the subject is 80 years old or greater. In some embodiments, the subject is 85 years old or greater.

In some embodiments, the subject has a body mass index (BMI) of 25, 26, 27, 28, 29, 30, 31, 32, 33, 34, 35, 36, 37, 38, 39, 40, 41, 42, 43, 44, 45, 46, 47, 48, 49, 50, or more, or a range defined by any two of the aforementioned integers. In some embodiments, the subject is overweight. In some embodiments, the subject has a BMI of 25 or more. In some embodiments, the subject has a BMI of 25-29. In some embodiments, the subject is obese. In some embodiments, the subject has a BMI of 30 or more. In some embodiments, the subject has a BMI of 30-39. In some embodiments, the subject has a BMI of 40-50. In some embodiments, the subject has a BMI of 25-50.

In some embodiments, the subject has a personal history with the disorder. In some embodiments, the subject has a familial history with the disorder. In some embodiments, the subject is at high risk of contracting the disorder.

In some embodiments, the subject has a coagulation or clotting disorder. In some embodiments, the coagulation or clotting disorder comprises Hemophilia, Von Willebrand disease or clotting factor deficiencies. In some embodiments, the subject has a thrombophilia. In some embodiments, the thrombophilia comprises an inherited thrombophilia. In some embodiments, the thrombophilia comprises an acquired thrombophilia. In some embodiments, the subject has a hypercoagulable state. In some embodiments, the hypercoagulable state comprises cancer. In some embodiments, the hypercoagulable state comprises atrial fibrillation. In some embodiments, the hypercoagulable states comprises a post surgical period or immobility. In some embodiments, the subject has arterial thrombosis or thromboembolism. In some embodiments, the subject has venous thrombosis or thromboembolism. In some embodiments, the venous thromboembolism comprises deep venous thrombosis. In some embodiments, the venous thromboembolism comprises pulmonary embolism. In some embodiments, the venous thromboembolism comprises thrombophlebitis.

### C. Baseline Measurements

Some embodiments of the methods described herein include obtaining a baseline measurement from a subject. In some embodiments, the baseline measurement is a mental disorder (e.g., psychiatric or neurological disorder) baseline measurement. For example, in some embodiments, a baseline measurement is obtained from the subject prior to treating the subject. Non-limiting examples of baseline measurements include a baseline measurement of Montgomery-Asberg Depression Rating Scale (MADRS); a baseline Hamilton Depression Rating Scale-17 (e.g., scale ranges from 0 to 52 with a higher score indicating worsening symptoms of depression); baseline anxiety symptoms and/or signs, baseline eating disorder symptoms and/or signs, baseline substance-use disorder symptoms and/or signs, baseline post-traumatic stress disorder symptoms and/or signs, baseline bipolar disorder symptoms and/or signs, baseline schizophrenia symptoms and/or signs, and baseline psychosis symptoms and/or signs. In some embodiments, the baseline measurement includes an aspect of any of Tables 1A-1C and 2A-2B. The baseline measurement may include a baseline fibrinogen measurement, a baseline FGG mRNA measurement, or a baseline FGG protein measurement. The baseline measurement may include a baseline clotting measurement, a baseline prothrombin time (PT) measurement, a baseline Intemational Normalized Ratio (INR) measurement, or a baseline activated partial thromboplastin time (aPTT) measurement.

In some embodiments, the baseline measurement is obtained directly from the subject. In some embodiments, the baseline measurement is obtained by observation of the subject. In some embodiments, the baseline measurement is obtained by questioning the subject. In some embodiments, the baseline measurement is obtained by the subject filling out a questionnaire.

In some embodiments, the baseline measurement is a baseline Montgomery-Asberg Depression Rating Scale (MADRS) score. The MADRS scale may range from 0 to 60 with a higher score indicating worsening symptoms of depression. The MADRS generally includes a ten-item diagnostic questionnaire which psychiatrists use to measure the severity of depressive episodes in patients with mood disorders. It was designed as an adjunct to the HDRS to be, in some cases, more sensitive to changes brought on by antidepressants or other forms of treatment. A higher MADRS score indicates more severe depression than a lower score. The overall score ranges from 0 to 60. Example cutoff points are as follows:
0 to 6—normal/absent symptoms
7 to 19—mild depression
20 to 34—moderate depression, and
>34—severe depression.

In some embodiments, the baseline MADRS score comprises a numerical value such as a number of points. In some embodiments, the numerical value is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 41, 42, 43, 44, 45, 46, 47, 48, 49, 50, 51, 52, 53, 54, 55 56, 57, 58, 59, or 60, or a range defined by any two of the aforementioned numerical values. In some embodiments, the numerical value is 1-5. In some embodiments, the numerical value is 6-10. In some embodiments, the numerical value is 11-15. In some embodiments, the numerical value is 16-20. In some embodiments, the numerical value is 21-25. In some embodiments, the numerical value is 26-30. In some embodiments, the numerical value is 31-35. In some embodiments, the numerical value is 36-40. In some embodiments, the numerical value is 41-45. In some embodiments, the numerical value is 46-50. In some embodiments, the numerical value is 51-55. In some embodiments, the numerical value is 56-60. In some embodiments, the numerical value is 1-60. In some embodiments, the baseline MADRS score comprises a baseline subscore such as a baseline apparent sadness score, a baseline reported sadness score, a baseline inner tension score, a baseline reduced sleep score, a baseline reduced appetite score, a baseline concentration difficulties score, a baseline lassitude score, a baseline inability to feel score, a baseline pessimistic thoughts score, or a baseline suicidal thoughts score. Each baseline subscore may comprise a numerical value of 0, 1, 2, 3, 4, 5, or 6, or a range of such numerical values. In some embodiments, the baseline MADRS score comprises a numerical value at or above a threshold numerical value that is indicative of a depressive disorder. For example, the subject may be depressed prior to treatment and have a baseline MADRS score of 7-60. The subject may have mild depression prior to treatment and have a baseline MADRS score of 7-19. The subject may have moderate depression prior to treatment and have a baseline MADRS score of 20-34. The subject may have severe depression prior to treatment and have a baseline MADRS score over 34. In some embodiments, one or more of the baseline subscores comprise a numerical value at or above a threshold numerical value that is indicative of the depressive disorder.

In some embodiments, the baseline measurement comprises a baseline Hamilton Depression Rating Scale (HDRS) score. The HRSD typically includes a multiple item questionnaire used to provide an indication of depression, and as a guide to evaluate recovery. The questionnaire is usually designed for adults and is used to rate the severity of their depression by probing mood, feelings of guilt, suicide ideation, insomnia, agitation or retardation, anxiety, weight loss, and somatic symptoms. The subject is usually rated by a clinician on 17 to 29 items (depending on version) scored either on a 3-point or 5-point Likert-type scale. In some cases, the HDRS includes 17 items (HDRS₁₇). Other variations may be used, such as those that include more than 17 items. For example, up to 29 items may be used in some cases (HDRS₂₉). For the 17-item version, a score of 0-7 is typically considered to be normal while a score of 20 or higher may indicate moderate or severe depression.

In some embodiments, the baseline HDRS score comprises a numerical value such as a number of points. In some embodiments, the numerical value is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 41, 42, 43, 44, 45, 46, 47, 48, 49, or 50, or a range defined by any two of the aforementioned numerical values. In some embodiments, the numerical value is 1-5. In some embodiments, the numerical value is 6-10. In some embodiments, the numerical value is 11-15. In some embodiments, the numerical value is 16-20. In some embodiments, the numerical value is 21-25. In some embodiments, the numerical value is 26-30. In some embodiments, the numerical value is 31-35. In some embodiments, the numerical value is 36-40. In some embodiments, the numerical value is 41-45. In some embodiments, the numerical value is 46-50. In some embodiments, the numerical value is 51 or 52. In some embodiments, the numerical value is 1-50. In some embodiments, the numerical value is 1-52. In some embodiments, the baseline HDRS score comprises a baseline subscore such as a baseline depressed mood score, a baseline feelings of guilt score, a baseline suicide score, a baseline insomnia early in the night score, a baseline insomnia in the middle of the night score, a baseline insomnia in early hours of the morning score, a baseline work and activities score, a baseline retardation score, a baseline agitation score, a baseline anxiety psychic score, a baseline anxiety somatic score, a baseline somatic symptoms of gastrointestinal score, a baseline general somatic score, a baseline genital symptoms score, a baseline hypochondriasis score, a baseline loss of weight score, or a baseline insight score. Baseline subscores may comprise a numerical value of 0, 1, or 2, or a range of such numerical values. Baseline subscores may comprise a numerical value of 0, 1, 2, 3, or 4, or a range of such numerical values. In some embodiments, the baseline HDRS score comprises a numerical value at or above a threshold numerical value that is indicative of a depressive disorder. For example, a HDRS score of 20 or higher may be indicative of moderate to severe depression. In some cases, the subject is depressed prior to treatment and has an HDRS score above 19. In some cases, the subject is at least mildly depressed prior to treatment and has an HDRS score above 7. In some embodiments, the baseline subscore comprises a numerical value at or above a threshold numerical value that is indicative of the depressive disorder.

In some embodiments, the baseline measurement is a baseline anxiety measurement. The baseline anxiety measurement may include a baseline assessment of a sign or symptom of anxiety (e.g., a baseline anxiety sign or symptom). Examples of signs or symptoms of anxiety include stress (e.g. stress that's out of proportion to the impact of an event), worry (for example, inability to set aside a worry), or restlessness. In some cases, the symptom of anxiety includes one or more behavioral symptoms such as hypervigilance, irritability, or restlessness. In some cases, the symptom of anxiety includes one or more cognitive symptoms such as lack of concentration, racing thoughts, or unwanted thoughts. In some cases, the symptom of anxiety includes one or more whole body symptoms such as fatigue or sweating. In some cases, the symptoms of anxiety include any of excessive worry, fear, feeling of impending doom, insomnia, nausea, palpitations, or trembling. In some embodiments, the symptom includes one or more panic attacks. The baseline assessment may include an amount, frequency, duration, or intensity of the anxiety or symptoms of anxiety. The baseline assessment may include an amount of time since experiencing the anxiety or symptoms. The baseline assessment may include a frequency of experiencing the anxiety or symptoms.

In some embodiments, the baseline measurement is a baseline eating disorder measurement. In some embodiments, the baseline measurement is a baseline eating disorder sign or symptom. Examples of eating disorders include anorexia, bulimia, binge eating disorder, pica, rumination, or avoidant eating disorder. In some embodiments, the eating disorder includes anorexia nervosa. In some embodiments, the eating disorder includes bulimia. In some embodiments, the eating disorder includes binge eating. In some embodiments, the eating disorder includes pica. The baseline eating disorder measurement may include a baseline assessment of a sign or symptom of eating disorder (e.g., a baseline eating disorder sign or symptom). Some examples of symptoms of an eating disorder comprising anorexia nervosa include being considerably underweight compared with people of similar age and height, very restricted eating patterns, an intense fear of gaining weight or persistent behaviors to avoid gaining weight despite being underweight, a relentless pursuit of thinness and unwillingness to maintain a healthy weight, a heavy influence of body weight or perceived body shape on self-esteem, a distorted body image, or denial of being seriously underweight. The baseline assessment may include an amount, frequency, duration, or intensity of the engaging in the eating disorder or experiencing symptoms of the eating disorder. The baseline assessment may include an amount of time since engaging in the eating disorder or experiencing symptoms of the eating disorder. The baseline assessment may include a frequency of engaging in the eating disorder or experiencing symptoms of the eating disorder.

In some embodiments, the baseline measurement is a baseline substance-use measurement. In some embodiments, the baseline substance-use measurement includes a baseline determination of a level of addiction to an addictive substance. Examples of addictive substances include alcohol, antianxiety drugs, sedative drugs, caffeine, cannabis (e.g. including marijuana or synthetic cannabinoids), hallucinogens (e.g. LSD, phencyclidine, or psilocybin), inhalants (e.g. paint thinner or some glues), opioids (e.g. fentanyl, morphine, or oxycodone), stimulants (e.g. amphetamines or cocaine), tobacco, or anabolic steroids. The baseline determination of a level of addiction to an addictive substance may include a questionnaire or assessment. The baseline determination of a level of addiction to an addictive substance may include an amount of time since ingesting the addictive substance. The baseline determination of a level of addiction to an addictive substance may include a frequency of ingesting the addictive substance. The baseline assessment may include an amount, frequency, duration, or intensity of the engaging in the substance-use disorder or experiencing symptoms of the substance-use disorder. The baseline assessment may include an amount of time since engaging in the substance-use disorder or experiencing symptoms of the substance-use disorder. The baseline assessment may include a frequency of engaging in the substance-use disorder or experiencing symptoms of the substance-use disorder. The baseline assessment may include signs or symptoms of the substance-use disorder. Exemplary signs and symptoms may include feelings of regularly (e.g., daily) substance-use, intense urges for the substance, needing more of the substance to obtain a previously obtained effect, and continuing to use the substance although use of the substance is known to cause problems in normal life activities.

In some embodiments, the baseline measurement comprises a baseline post-traumatic stress disorder (PTSD) measurement. In some embodiments, the baseline PTSD measurement includes a baseline determination of the level of severity of PTSD. The baseline assessment of a sign or symptom of PTSD may include the number of signs or symptoms of PTSD. The baseline determination of the level of severity of PTSD may include the time since last experiencing a PTSD flashback (e.g., reliving the traumatic event as if it were happening again), nightmare, or severe anxiety. The baseline assessment may include a frequency in PTSD related flashbacks, nightmares, or severe anxiety episodes. The baseline assessment may include a severity of a sign or symptom of PTSD. The baseline assessment may include a frequency of a sign or symptom of PTSD. Exemplary signs and symptoms of PTSD may include intrusive memories (e.g., recurrent, unwanted distressing memories of atraumatic event, severe emotional distress or physical reactions to something that reminiscent of the traumatic event, attempts to avoid thinking or talking about the traumatic event, avoiding places, activities or people reminiscent of the traumatic event, thoughts of hopelessness, memory problems, difficulty maintaining close relationships, and feeling a lack of interest in activities that were once enjoyed.

In some embodiments, the baseline measurement comprises a baseline bipolar disorder measurement. In some embodiments, the baseline bipolar disorder measurement is a sign or symptom of bipolar disorder. The baseline assessment of a sign or symptom of bipolar disorder may include a frequency of a sign or symptom of bipolar disorder. The baseline assessment of a sign or symptom of bipolar disorder may include a severity of a sign or symptom of bipolar disorder. The baseline assessment of a sign or symptom of bipolar disorder may include the number of signs or symptoms of bipolar disorder. Exemplary signs and symptoms of bipolar disorder include any of the bipolar signs and symptoms disclosed herein, including, manic episodes (e.g., experiencing feelings of increased activity, energy, or agitation, an exaggerated sense of well-being and self-confidence, a decreased need for sleep, racing thoughts, distractibility, and a decreased ability to control impulses), and major depressive episodes (e.g., experiencing a depressed mood, marked loss of interest of feelings of pleasure, fatigue or loss of energy, feelings of guilt or worthlessness, and a decreased ability to think or concentrate).

In some embodiments, the baseline measurement comprises a baseline schizophrenia measurement. In some embodiments, the baseline schizophrenia measurement is a sign or symptom of schizophrenia. The baseline assessment of a sign or symptom of schizophrenia may include a frequency of a sign or symptom of schizophrenia. The baseline assessment of a sign or symptom of schizophrenia may include a severity of a sign or symptom of schizophrenia. The baseline assessment of a sign or symptom of schizophrenia may include the number of signs or symptoms of schizophrenia. Exemplary signs and symptoms of schizophrenia may include delusions, hallucinations, disorganized thoughts and speech, disorganized or abnormal motor behavior, and negative symptoms (e.g., social withdrawal, anhedonia, avolition, decreased sense of purpose, lack of interest in activities, flat affect, lack of eye contact, and physical inactivity.

In some embodiments, the baseline measurement comprises a baseline psychosis measurement. In some embodiments, the baseline psychosis measurement is a baseline sign or symptom of psychosis (e.g., baseline psychosis sign or symptom). The baseline assessment of a sign or symptom of psychosis may include a frequency of a sign or symptom of psychosis. The baseline assessment of a sign or symptom of psychosis may include a severity of a sign or symptom of psychosis. The baseline assessment of a sign or symptom of psychosis may include the number of signs or symptoms of psychosis. Exemplary signs and symptoms of psychosis may include difficulty concentrating, depressed mood, anxiety, excessive suspiciousness, delusions, and hallucinations.

In some embodiments, the baseline measurement comprises a baseline measurement of a neurological disorder. Non-limiting examples of baseline measurements of neurological disorders include a baseline measurement of cognitive function, a baseline measurement of CNS amyloid plaque(s) (e.g., accumulation), a baseline measurement of CNS tau accumulation, a baseline measurement of CSF beta-amyloid 42 (e.g., accumulation), a baseline measurement of CSF tau (e.g., accumulation), a baseline measurement of CSF phospho-tau (e.g., accumulation), a baseline measurement of Lewy bodies (e.g., accumulation), or a baseline measurement of CSF alpha-synuclein (e.g., accumulation). Further non-limiting examples of baseline measurements include a baseline measurement of headache signs and/or symptoms, a baseline measurement of migraine symptoms and/or signs, a baseline measurement of chronic pain symptoms and/or signs, a baseline measurement of fibromyalgia symptoms and/or signs, a baseline measurement of chronic fatigue syndrome (ME) symptoms and/or signs, and a baseline measurement of motor neuron disease (e.g., ALS) symptoms and/or signs.

In some embodiments, the baseline measurement comprises a baseline CNS amyloid plaque accumulation measurement. Exemplary CNS amyloid plaque accumulation measurements may include a total amount of amyloid plaque in the CNS (e.g., the brain) and the concentration of amyloid plaque in an area of the CNS (e.g., the brain). CNS amyloid plaque accumulation may be measured in any appropriate manner, including, but not limited to a measurement of CNS amyloid plaque through the use of immunoprecipitation mass spectrometry, blood tests, cerebrospinal fluid tests, and a measurement of CNS amyloid plaque through the use of imaging (e.g., amyloid PET scan(s)).

In some embodiments, the baseline measurement comprises a baseline tau (e.g., CNS or CSF) accumulation. Exemplary tau (e.g., CNS or CSF) accumulation measurements may include a total amount of tau accumulation in the CNS (e.g., the brain or CSF), and the concentration of tau in an area of the CNS (e.g., the brain or CSF). Tau (e.g., CNS or CSF) accumulation may be measured in any appropriate manner, including, but not limited to imaging (e.g., tau PET scans), blood tests, and cerebrospinal fluid tests.

In some embodiments, the baseline measurement comprises a baseline CSF beta-amyloid 42 accumulation. Exemplary CSF beta-amyloid 42 accumulation measurements may include a total amount of beta-amyloid 42 accumulation in the CNS (e.g., the CSF), and the concentration of beta-amyloid 4 in an area of the CNS (e.g., CSF). CSF beta-amyloid 4 accumulation may be measured in any appropriate manner, including, but not limited to imaging (e.g., PET scans), blood tests, and cerebrospinal fluid tests.

In some embodiments, the baseline measurement comprises a baseline CSF beta-amyloid 42 accumulation. Exemplary CSF beta-amyloid 42 accumulation measurements may include a total amount of beta-amyloid 42 accumulation in the CNS (e.g., the CSF), and the concentration of beta-amyloid 4 in an area of the CNS (e.g., CSF). CSF beta-amyloid 4 accumulation may be measured in any appropriate manner, including, but not limited to imaging (e.g., PET scans), blood tests, and cerebrospinal fluid tests.

In some embodiments, the baseline measurement comprises a baseline (e.g., CSF) phospho-tau accumulation. Exemplary (e.g., CSF) phospho-tau (e.g., 181) accumulation measurements may include a total amount of phospho-tau accumulation in the CNS (e.g., the CSF or the brain), and the concentration of (e.g., CSF) phospho-tau in an area of the CNS (e.g., CSF or the brain). Phospho-tau (e.g., CSF phosphor-tau) accumulation may be measured in any appropriate manner, including, but not limited to imaging (e.g., PET scans), blood tests, and cerebrospinal fluid tests.

In some embodiments, the baseline measurement comprises a baseline Lewy body accumulation. Exemplary Lewy body accumulation measurements may include a total amount of Lewy body accumulation in the CNS (e.g., the brain), and the concentration of Lewy body in an area of the CNS (e.g., brain). Lewy body accumulation may be measured in any appropriate manner, including, but not limited to imaging (e.g., PET scan, MRI, CT scan, fluorodeoxyglucose PET scan, or single-photon emission computerized tomography (SPECT)), blood tests, and cerebrospinal fluid tests.

In some embodiments, the baseline measurement comprises a baseline (e.g., CSF) alpha-synuclein accumulation. Exemplary (e.g., CSF) alpha-synuclein accumulation measurements may include a total amount of alpha-synuclein accumulation in the CNS (e.g., the CSF or the brain), and the concentration of (e.g., CSF) alpha-synuclein in an area of the CNS (e.g., CSF or the brain). Alpha-synuclein (e.g., CSF alpha-synuclein) accumulation may be measured in any appropriate manner, including, but not limited to imaging (e.g., PET scans), blood tests, cerebrospinal fluid tests, and biopsy tests (e.g., Syn-One test).

In some embodiments, the baseline measurement is a baseline cognitive function measurement. The baseline cognitive function measurement may be obtained directly from the subject. For example, the subject may be administered a test. The test may include a cognitive test such as the Montreal Cognitive Assessment (MoCA), Mini-Mental State Exam (MMSE), or Mini-Cog. The test may include assessment of basic cognitive functions such as memory, language, executive frontal lobe function, apraxia, visuospatial ability, behavior, mood, orientation, or attention. The baseline cognitive function measurement may include a score. The baseline cognitive function measurement may be indicative of mild cognitive impairment, or of severe cognitive impairment. The baseline cognitive function measurement may be indicative of a neurological disorder.

In some embodiments, the baseline measurement is a baseline amyloid plaque measurement. The baseline amyloid plaque measurement may include a central nervous system (CNS) amyloid plaque measurement. In some embodiments, the baseline amyloid plaque measurement includes a baseline concentration or amount. The baseline amyloid plaque measurement may be performed using an imaging device. The imaging device may include a positron emission tomography (PET) device. The baseline amyloid plaque measurement may be performed on a biopsy. The baseline amyloid plaque measurement may be performed using a spinal tap (for example, when the baseline amyloid plaque measurement includes a baseline cerebrospinal fluid (CSF) amyloid plaque measurement). In some embodiments, the baseline amyloid plaque measurement is obtained by an assay such as an immunoassay. The baseline beta amyloid plaque measurement may be indicative of a neurodegenerative disease such as Alzheimer's disease.

In some embodiments, the baseline measurement is a baseline beta-amyloid 42 measurement. The baseline beta-amyloid 42 measurement may include a cerebrospinal fluid (CSF) beta-amyloid 42 measurement. In some embodiments, the baseline beta-amyloid 42 measurement includes a baseline concentration or amount. The baseline beta-amyloid 42 measurement may be performed on a biopsy. The baseline beta-amyloid 42 measurement may be performed using a spinal tap (for example, when the baseline beta-amyloid 42 measurement includes a baseline CSF beta-amyloid 42 measurement). In some embodiments, the baseline beta-amyloid 42 measurement is obtained by an assay such as an immunoassay. The baseline beta-amyloid 42 measurement may be indicative of a neurodegenerative disease such as Alzheimer's disease.

In some embodiments, the baseline measurement is a baseline tau measurement. In some embodiments, the baseline tau measurement includes a baseline concentration or amount. The baseline tau measurement may be performed on a biopsy. In some embodiments, the baseline tau measurement is obtained by an assay such as an immunoassay. The baseline beta tau measurement may be indicative of a neurodegenerative disease such as Alzheimer's disease or Parkinson's disease.

In some embodiments, the baseline tau measurement is a baseline central nervous system (CNS) tau measurement. The baseline tau measurement may include a baseline total tau measurement. The baseline tau measurement may include a baseline unphosphorylated tau measurement. The baseline tau measurement may include a baseline phosphorylated tau (phospho-tau) measurement. In some embodiments, the baseline tau measurement is a baseline tau accumulation measurement. In some embodiments, the baseline tau measurement is a baseline CNS tau accumulation measurement. The baseline CNS tau accumulation measurement may be indicative of a neurodegenerative disease such as Alzheimer's disease or Parkinson's disease.

The baseline tau measurement may include a cerebrospinal fluid (CSF) tau measurement. The baseline CSF tau measurement may be performed after use of a spinal tap. The baseline CSF tau measurement may be indicative of a neurodegenerative disease such as Alzheimer's disease or Parkinson's disease.

The baseline CSF tau measurement may include a baseline CSF phospho-tau measurement. The baseline CSF phospho-tau measurement may include an amount of phospho-tau in relation to total tau or unphosphorylated tau. For example, the baseline CSF phospho-tau measurement may include a phospho-tau/tau ratio. The baseline CSF phospho-tau measurement may be indicative of a neurodegenerative disease such as Alzheimer's disease or Parkinson's disease.

In some embodiments, the baseline measurement is a baseline Lewy body measurement. The baseline Lewy body measurement may include a central nervous system (CNS) Lewy body measurement. In some embodiments, the baseline Lewy body measurement includes a baseline concentration or amount. The baseline Lewy body measurement may be performed using an imaging device. The imaging device may include a positron emission tomography (PET) device. The baseline beta Lewy body measurement may be indicative of dementia.

In some embodiments, the baseline measurement is a baseline alpha-synuclein measurement. The baseline alpha-synuclein measurement may include a cerebrospinal fluid (CSF) alpha-synuclein measurement. In some embodiments, the baseline alpha-synuclein measurement includes a baseline concentration or amount. The baseline alpha-synuclein measurement may be performed on a biopsy. The baseline alpha-synuclein measurement may be performed using a spinal tap (for example, when the baseline alpha-synuclein measurement includes a baseline CSF alpha-synuclein measurement). In some embodiments, the baseline alpha-synuclein measurement is obtained by an assay such as an immunoassay. The baseline alpha-synuclein measurement may be indicative of a neurodegenerative disease such as Parkinson's disease. The baseline alpha-synuclein measurement may be indicative of dementia.

In some embodiments, the baseline measurement is a baseline headache measurement. some embodiments, the baseline headache measurement is a baseline headache sign or symptom measurement. In some embodiments, the baseline headache measurement is a baseline migraine (e.g., with aura or without aura) measurement. In some embodiments, the baseline headache measurement is a frequency of a headache sign or symptom measurement. In some embodiments, the baseline headache measurement is a severity of a headache sign or symptom measurement. In some embodiments, the baseline headache measurement is a number of headache signs or symptoms. Exemplary signs and symptoms of headaches include pain (e.g., deep and constant) in the cheekbones, forehead, bridge of the nose, the cranium, or the back of the neck, aura, photophobia, phonophobia, and emesis.

In some embodiments, the baseline measurement is a baseline chronic pain measurement. In some embodiments, baseline chronic pain measurement is a baseline fibromyalgia measurement. In some embodiments, the baseline chronic pain measurement is a baseline chronic pain sign or symptom measurement. In some embodiments, the baseline chronic pain measurement is a frequency of a chronic pain sign or symptom measurement. In some embodiments, the baseline chronic pain measurement is a severity of a chronic pain sign or symptom measurement. In some embodiments, the baseline chronic pain measurement is a number of chronic pain signs or symptoms. Exemplary signs and symptoms of fibromyalgia include muscular pain, fatigues, depression, anxiety, sleeplessness, headache, and difficulty concentrating. Exemplary chronic pain disorders include postsurgical pain, post-trauma pain, low back pain, cancer pain, arthritis pain, muscular pain, and neuropathic pain (e.g., diabetic neuropathy).

In some embodiments, the baseline measurement is a baseline chronic fatigue syndrome (also referred to as myalgic encephalomyelitis) measurement. In some embodiments, the baseline chronic fatigue syndrome measurement is a baseline chronic fatigue syndrome sign or symptom measurement. In some embodiments, the baseline chronic fatigue syndrome measurement is a frequency of a headache sign or symptom measurement. In some embodiments, the baseline chronic fatigue syndrome measurement is a severity of a chronic fatigue syndrome sign or symptom measurement. In some embodiments, the baseline chronic fatigue syndrome n measurement is a number of chronic fatigue syndrome signs or symptoms. Exemplary signs and symptoms of chronic fatigue syndrome include extreme fatigue that lasts for extended periods of time (e.g., for at least six months) that cannot be fully explained by an underlying medical condition, fatigue that worsens with physical or mental activity, pain (e.g., joint or muscular), malaise, forgetfulness, anxiety, and depression.

In some embodiments, the baseline measurement is a baseline motor neuron disease measurement. In some embodiments, the baseline motor neuron disease measurement is an amyotrophic lateral sclerosis (ALS) measurement. In some embodiments, the baseline motor neuron disease measurement is a baseline motor neuron disease sign or symptom measurement. In some embodiments, the baseline motor neuron disease measurement is a frequency of a motor neuron disease sign or symptom measurement. In some embodiments, the baseline motor neuron disease measurement is a severity of a motor neuron disease sign or symptom measurement. In some embodiments, the baseline motor neuron disease measurement is a number of motor neuron disease signs or symptoms. Exemplary forms of motor neuron diseases include progressive bulbar palsy (PBP), progressive muscular atrophy (PMA), ALS, and primary lateral sclerosis (PLS). Exemplary signs and symptoms of motor neuron diseases include motor control difficulties (e.g., difficulty walking or completing normal daily activities), muscular weakness, slurred speech, difficulty swallowing, and muscle cramps and twitching (e.g., in the arms, shoulders, or tongue).

In some embodiments, the baseline measurement is a baseline level of fibrinogen. In some embodiments, the baseline measurement is a baseline level of circulating fibrinogen.

In some embodiments, the baseline measurement is a baseline clotting or coagulation measurement. In some embodiments, the baseline measurement is a baseline clotting time measurement. In some embodiments, the baseline measurement is a baseline prothrombin time (PT). In some embodiments, the baseline measurement is a baseline International Normalized Ratio (INR). In some embodiments, the baseline measurement is a baseline activated partial thromboplastin time (aPTT).

In some cases, the disorder (e.g., baseline measurement) may be diagnosed or measured with the use of a questionnaire or a scoring system. In some cases, the disorder is diagnosed according to DSM-5 criteria. In some cases, the disorder is diagnosed by a healthcare professional (e.g., physician or the like).

Baseline measurements may include a baseline FGG protein measurement, or a baseline FGG mRNA measurement.

Baseline measurements may include any one or more of the baseline measurements disclosed herein.

In some embodiments, the baseline measurement is obtained directly from the subject. In some embodiments, the baseline measurement is obtained by observation, for example by observation of the subject or of the subject's tissue. In some embodiments, the baseline measurement is obtained noninvasively using an imaging device. In some embodiments, the baseline measurement is obtained invasively using an imaging device.

In some embodiments, the baseline measurement is obtained in a sample from the subject. In some embodiments, the baseline measurement is obtained in one or more histological tissue sections. In some embodiments, the baseline measurement is obtained by performing an assay such as an immunoassay, a colorimetric assay, or a fluorescence assay, on the sample obtained from the subject. In some embodiments, the baseline measurement is obtained by an immunoassay, a colorimetric assay, a fluorescence assay, or a chromatography (e.g. HPLC) assay. In some embodiments, the baseline measurement is obtained by PCR.

In some embodiments, the baseline measurement is a baseline FGG protein measurement. In some embodiments, the baseline FGG protein measurement comprises a baseline FGG protein level. In some embodiments, the baseline FGG protein level is indicated as a mass or percentage of FGG protein per sample weight. In some embodiments, the baseline FGG protein level is indicated as a mass or percentage of FGG protein per sample volume. In some embodiments, the baseline FGG protein level is indicated as a mass or percentage of FGG protein per total protein within the sample. In some embodiments, the baseline FGG protein measurement is a baseline tissue FGG protein measurement. In some embodiments, the baseline FGG protein measurement is obtained by an assay such as an immunoassay, a colorimetric assay, or a fluorescence assay. In some embodiments, the baseline FGG protein level is measured in the whole body. In some embodiments, the baseline FGG protein level is measured in the brain. In some embodiments, the baseline FGG protein level is measured in the liver. In some embodiments, the baseline FGG protein level is measured in the blood.

In some embodiments, the baseline measurement is a baseline FGG mRNA measurement. In some embodiments, the baseline FGG mRNA measurement comprises a baseline FGG mRNA level. In some embodiments, the baseline FGG mRNA level is measured in the liver. In some embodiments, the baseline FGG mRNA level is indicated as an amount or percentage of FGG mRNA per sample weight. In some embodiments, the baseline FGG mRNA level is indicated as an amount or percentage of FGG mRNA per sample volume. In some embodiments, the baseline FGG mRNA level is indicated as an amount or percentage of FGG mRNA per total mRNA within the sample. In some embodiments, the baseline FGG mRNA level is indicated as an amount or percentage of FGG mRNA per total nucleic acids within the sample. In some embodiments, the baseline FGG mRNA level is indicated relative to another mRNA level, such as an mRNA level of a housekeeping gene, within the sample. In some embodiments, the baseline FGG mRNA measurement is a baseline tissue FGG mRNA measurement. In some embodiments, the baseline FGG mRNA measurement is obtained by an assay such as a polymerase chain reaction (PCR) assay. In some embodiments, the PCR comprises quantitative PCR (qPCR). In some embodiments, the PCR comprises reverse transcription of the FGG mRNA.

Some embodiments of the methods described herein include obtaining a sample from a subject. In some embodiments, the baseline measurement is obtained in a sample obtained from the subject. In some embodiments, the sample is obtained from the subject prior to administration or treatment of the subject with a composition described herein. In some embodiments, a baseline measurement is obtained in a sample obtained from the subject prior to administering the composition to the subject. In some embodiments, the sample is obtained from the subject in a fasted state. In some embodiments, the sample is obtained from the subject after an overnight fasting period. In some embodiments, the sample is obtained from the subject in a fed state.

In some embodiments, the sample comprises a fluid. In some embodiments, the sample is a fluid sample. In some embodiments, the sample is a blood, plasma, or serum sample. In some embodiments, the sample comprises blood. In some embodiments, the sample is a blood sample. In some embodiments, the sample is a whole-blood sample. In some embodiments, the blood is fractionated or centrifuged. In some embodiments, the sample comprises plasma. In some embodiments, the sample is a plasma sample. A blood sample may be a plasma sample. In some embodiments, the sample comprises serum. In some embodiments, the sample is a serum sample. A blood sample may be a serum sample. In some embodiments, the sample is a CSF sample. In some embodiments the sample includes a CSF sample. In some embodiments, the sample is a CNS sample. In some embodiments the sample includes a CNS sample.

In some embodiments, the sample comprises a tissue. In some embodiments, the sample is a tissue sample. In some embodiments, the tissue comprises liver or brain tissue. For example, the baseline FGG mRNA measurement, or the baseline FGG protein measurement, may be obtained in a brain or liver sample obtained from the patient. In some embodiments, the tissue comprises neural tissue. In some embodiments, the tissue comprises neuronal tissue. In some embodiments, the tissue comprises neurons. In some embodiments, the tissue comprises glial cells. In some embodiments, the tissue comprises epithelial cells. In some embodiments, the tissue comprises liver tissue. The liver may include hepatocytes. In some embodiments, the tissue comprises brain tissue. In some embodiments, the sample comprises CSF fluid.

In some embodiments, the sample includes cells. In some embodiments, the sample comprises a cell. In some embodiments, the cell comprises a liver cell (e.g., hepatocyte), or a brain cell. In some embodiments, the cell is a liver cell. In some embodiments, the liver cell is a hepatocyte. In some embodiments, the cell is a brain cell. In some embodiments, the cell is a neuron. In some embodiments, the cell is a glial cell. In some embodiments, the cell is an epithelial cell. In some embodiments, the cell is a vasculature cell.

### D. Effects

In some embodiments, the composition or administration of the composition affects a measurement such as mental disorder (e.g., psychiatric disorder or neurological disorder) measurement. In some embodiments, the composition or administration of the composition affects a measurement such as psychiatric measurement (e.g., a Montgomery-Asberg Depression Rating Scale (MADRS) score, a Hamilton Depression Rating Scale (HDRS) score, anxiety signs or symptoms, eating disorder signs or symptoms, substance-use disorder signs or symptoms, post-traumatic stress disorder (PTSD) signs or symptoms, bipolar disorder signs or symptoms, schizophrenia signs or symptoms, or psychosis signs or symptoms). In some embodiments, the composition or administration of the composition affects a measurement, such as psychiatric measurement, relative to the baseline measurement. In some embodiments, administration of the composition affects a measurement of an aspect in any of Tables 1A-1C and 2A-2B. The measurement may include a fibrinogen measurement, a FGG mRNA measurement, or a FGG protein measurement. The measurement may include a clotting measurement, a prothrombin time (PT) measurement, an International Normalized Ratio (INR) measurement, or a activated partial thromboplastin time (aPTT) measurement.

In some embodiments, the composition or administration of the composition affects a measurement such as neurological measurement (e.g., decreased cognitive function, CNS amyloid plaques (e.g., accumulation), CNS tau accumulation, CSF beta-amyloid 42 (e.g., accumulation), CSF tau (e.g., accumulation), CSF phospho-tau (e.g., accumulation), Lewy bodies (e.g., accumulation), CSF alpha-synuclein (e.g., accumulation), headache signs or symptoms, migraine signs or symptoms, chronic pain signs or symptoms, fibromyalgia signs or symptoms, chronic fatigue (ME) signs or symptoms, motor neuron disease signs or symptoms, or ALS signs or symptoms). In some embodiments, the composition or administration of the composition affects a measurement, such as neurological measurement, relative to the baseline measurement.

In some embodiments, the measurement indicates that the disorder has been treated. In some embodiments, the measurement indicates that the severity of the disorder has decreased. In some embodiments, the measurement indicates that the severity of a sign or symptom of the disorder has decreased. In some embodiments, the measurement indicates that the frequency of a sign or symptom of the disorder has decreased.

Some embodiments of the methods described herein include obtaining the measurement from a subject. For example, the measurement may be obtained from the subject after treating the subject. In some embodiments, the measurement is obtained in a second sample (such as a fluid or tissue sample described herein) obtained from the subject after the composition is administered to the subject. In some embodiments, the measurement is an indication that the disorder has been treated.

In some embodiments, the measurement is obtained directly from the subject. In some embodiments, the measurement is obtained noninvasively using an imaging device. In some embodiments, the measurement is obtained in a second sample from the subject. In some embodiments, the measurement is obtained in one or more histological tissue sections. In some embodiments, the measurement is obtained by performing an assay on the second sample obtained from the subject. In some embodiments, the measurement is obtained by an assay, such as an assay described herein. In some embodiments, the assay is an immunoassay, a colorimetric assay, a fluorescence assay, a chromatography (e.g. HPLC) assay, or a PCR assay. In some embodiments, the measurement is obtained by an assay such as an immunoassay, a colorimetric assay, a fluorescence assay, or a chromatography (e.g. HPLC) assay. In some embodiments, the measurement is obtained by PCR. In some embodiments, the measurement is obtained by histology. In some embodiments, the measurement is obtained by observation. In some embodiments, additional measurements are made, such as in a 3rd sample, a 4th sample, or a fifth sample.

In some embodiments, the measurement is obtained within 1 hour, within 2 hours, within 3 hours, within 4 hours, within 5 hours, within 6 hours, within 12 hours, within 18 hours, or within 24 hours after the administration of the composition. In some embodiments, the measurement is obtained within 1 day, within 2 days, within 3 days, within 4 days, within 5 days, within 6 days, or within 7 days after the administration of the composition. In some embodiments, the measurement is obtained within 1 week, within 2 weeks, within 3 weeks, within 1 month, within 2 months, within 3 months, within 6 months, within 1 year, within 2 years, within 3 years, within 4 years, or within 5 years after the administration of the composition. In some embodiments, the measurement is obtained after 1 hour, after 2 hours, after 3 hours, after 4 hours, after 5 hours, after 6 hours, after 12 hours, after 18 hours, or after 24 hours after the administration of the composition. In some embodiments, the measurement is obtained after 1 day, after 2 days, after 3 days, after 4 days, after 5 days, after 6 days, or after 7 days after the administration of the composition. In some embodiments, the measurement is obtained after 1 week, after 2 weeks, after 3 weeks, after 1 month, after 2 months, after 3 months, after 6 months, after 1 year, after 2 years, after 3 years, after 4 years, or after 5 years, following the administration of the composition.

In some embodiments, the composition reduces the measurement relative to the baseline measurement. For example, an adverse phenotype of a psychiatric or neurological disorder may be reduced upon administration of the composition. In some embodiments, the reduction is measured in a second sample obtained from the subject after administering the composition to the subject. In some embodiments, the reduction is measured directly in the subject after administering the composition to the subject. In some embodiments, the measurement is decreased by about 2.5% or more, about 5% or more, or about 7.5% or more, relative to the baseline measurement. In some embodiments, the measurement is decreased by about 10% or more, relative to the baseline measurement. In some embodiments, the measurement is decreased by about 20% or more, about 30% or more, about 40% or more, about 50% or more, about 60% or more, about 70% or more, about 80% or more, about 90% or more, relative to the baseline measurement. In some embodiments, the measurement is decreased by no more than about 2.5%, no more than about 5%, or no more than about 7.5%, relative to the baseline measurement. In some embodiments, the measurement is decreased by no more than about 10%, relative to the baseline measurement. In some embodiments, the measurement is decreased by no more than about 20%, no more than about 30%, no more than about 40%, no more than about 50%, no more than about 60%, no more than about 70%, no more than about 80%, no more than about 90%, or no more than about 100% relative to the baseline measurement. In some embodiments, the measurement is decreased by 2.5%, 5%, 7.5%, 10%, 20%, 30%, 40%, 50%, 60%, 70%, 80%, 90%, or 100%, or by a range defined by any of the two aforementioned percentages.

In some embodiments, the composition increases the measurement relative to the baseline measurement. For example, a protective psychiatric or neurological phenotype may be increased upon administration of the composition. In some embodiments, the increase is measured in a second sample obtained from the subject after administering the composition to the subject. In some embodiments, the increase is measured directly in the subject after administering the composition to the subject. In some embodiments, the measurement is increased by about 2.5% or more, about 5% or more, or about 7.5% or more, relative to the baseline measurement. In some embodiments, the measurement is increased by about 10% or more, relative to the baseline measurement. In some embodiments, the measurement is increased by about 20% or more, about 30% or more, about 40% or more, about 50% or more, about 60% or more, about 70% or more, about 80% or more, about 90% or more, relative to the baseline measurement. In some embodiments, the measurement is increased by about 100% or more, increased by about 250% or more, increased by about 500% or more, increased by about 750% or more, or increased by about 1000% or more, relative to the baseline measurement. In some embodiments, the measurement is increased by no more than about 2.5%, no more than about 5%, or no more than about 7.5%, relative to the baseline measurement. In some embodiments, the measurement is increased by no more than about 10%, relative to the baseline measurement. In some embodiments, the measurement is increased by no more than about 20%, no more than about 30%, no more than about 40%, no more than about 50%, no more than about 60%, no more than about 70%, no more than about 80%, no more than about 90%, or no more than about 100% relative to the baseline measurement. In some embodiments, the measurement is increased by no more than about 100%, increased by no more than about 250%, increased by no more than about 500%, increased by no more than about 750%, or increased by no more than about 1000%, relative to the baseline measurement. In some embodiments, the measurement is increased by 2.5%, 5%, 7.5%, 10%, 20%, 30%, 40%, 50%, 60%, 70%, 80%, 90%, 100%, 250%, 500%, 750%, or 1000%, or by a range defined by any of the two aforementioned percentages.

In some embodiments, the measurement is a Montgomery-Asberg Depression Rating Scale (MADRS) score. In some embodiments, the MADRS score comprises a numerical value such as a number of points. In some embodiments, the numerical value is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 41, 42, 43, 44, 45, 46, 47, 48, 49, 50, 51, 52, 53, 54, 55 56, 57, 58, 59, or 60, or a range defined by any two of the aforementioned numerical values. In some embodiments, the numerical value is 0. In some embodiments, the numerical value is 1-5. In some embodiments, the numerical value is 6-10. In some embodiments, the numerical value is 11-15. In some embodiments, the numerical value is 16-20. In some embodiments, the numerical value is 21-25. In some embodiments, the numerical value is 26-30. In some embodiments, the numerical value is 31-35. In some embodiments, the numerical value is 36-40. In some embodiments, the numerical value is 41-45. In some embodiments, the numerical value is 46-50. In some embodiments, the numerical value is 51-55. In some embodiments, the numerical value is 56-60. In some embodiments, the numerical value is 0-60. In some embodiments, the MADRS score comprises a subscore such as a apparent sadness score, a reported sadness score, a inner tension score, a reduced sleep score, a reduced appetite score, a concentration difficulties score, a lassitude score, a inability to feel score, a pessimistic thoughts score, or a suicidal thoughts score. Each subscore may comprise a numerical value of 0, 1, 2, 3, 4, 5, or 6, or a range of such numerical values. In some embodiments, the MADRS score comprises a numerical value below a threshold numerical value that is indicative of a depressive disorder. In some embodiments, the subscore comprises a numerical value below a threshold numerical value that is indicative of a depressive disorder.

In some embodiments, the composition reduces the MADRS score relative to the baseline MADRS score. In some embodiments, the reduced MADRS score by observing and/or questioning the subject after administering the composition to the subject. In some embodiments, the MADRS score is decreased by about 2.5% or more, about 5% or more, or about 7.5% or more, relative to the baseline MADRS score. In some embodiments, the MADRS score is decreased by about 10% or more, relative to the baseline MADRS score. In some embodiments, the MADRS score is decreased by about 20% or more, about 30% or more, about 40% or more, about 50% or more, about 60% or more, about 70% or more, about 80% or more, about 90% or more, relative to the baseline MADRS score. In some embodiments, the MADRS score is decreased by no more than about 2.5%, no more than about 5%, or no more than about 7.5%, relative to the baseline MADRS score. In some embodiments, the MADRS score is decreased by no more than about 10%, relative to the baseline MADRS score. In some embodiments, the MADRS score is decreased by no more than about 20%, no more than about 30%, no more than about 40%, no more than about 50%, no more than about 60%, no more than about 70%, no more than about 80%, no more than about 90%, or no more than about 100% relative to the baseline MADRS score. In some embodiments, the MADRS score is decreased by 2.5%, 5%, 7.5%, 10%, 20%, 30%, 40%, 50%, 60%, 70%, 80%, 90%, or 100%, or by a range defined by any of the two aforementioned percentages. In some embodiments, the MADRS score is decreased by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 4041, 42, 43, 44, 45, 46, 47, 48, 49, 50, 51, 52, 53, 54, 55 56, 57, 58, 59, or 60 points, relative to the baseline MADRS score, or by a range of points defined by any two of the aforementioned numbers of points relative to the baseline MADRS score. In some embodiments, the MADRS score is decreased by 1-5 points. In some embodiments, the MADRS score is decreased by 6-10 points. In some embodiments, the MADRS score is decreased by 11-15 points. In some embodiments, the MADRS score is decreased by 16-20 points. In some embodiments, the MADRS score is decreased by 21-25 points. In some embodiments, the MADRS score is decreased by 26-30 points. In some embodiments, the MADRS score is decreased by 31-35 points. In some embodiments, the MADRS score is decreased by 36-40 points. In some embodiments, the MADRS score is decreased by 41-45 points. In some embodiments, the MADRS score is decreased by 46-50 points. In some embodiments, the MADRS score is decreased by 51-55 points. In some embodiments, the MADRS score is decreased by 56-60 points.

In some embodiments, following treatment with the oligonucleotide, the MADRS score of the subject is decreased such that the MADRS score of the subject changes from severe depression to mild or moderate depression, or to normal non-depressed symptomology. For example, the MADRS score of the subject may be below 35 following treatment. In some embodiments, the MADRS score changes from moderate depression to mild depression, or to normal non-depressed symptomology. For example, the MADRS score of the subject may be below 20 following treatment. In some embodiments, the MADRS score changes from mild depression to normal non-depressed symptomology. For example, the MADRS score of the subject may be below 7 following treatment.

In some embodiments, the measurement is a Hamilton Depression Rating Scale (HDRS) score. In some embodiments, the HDRS score comprises a numerical value such as a number of points. In some embodiments, the numerical value is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 41, 42, 43, 44, 45, 46, 47, 48, 49, or 50, or a range defined by any two of the aforementioned numerical values. In some embodiments, the numerical value is 0. In some embodiments, the numerical value is 1-5. In some embodiments, the numerical value is 6-10. In some embodiments, the numerical value is 11-15. In some embodiments, the numerical value is 16-20. In some embodiments, the numerical value is 21-25. In some embodiments, the numerical value is 26-30. In some embodiments, the numerical value is 31-35. In some embodiments, the numerical value is 36-40. In some embodiments, the numerical value is 41-45. In some embodiments, the numerical value is 46-50. In some embodiments, the numerical value is 0-50. In some embodiments, the HDRS score comprises a subscore such as a depressed mood score, a feelings of guilt score, a suicide score, a insomnia early in the night score, a insomnia in the middle of the night score, a insomnia in early hours of the morning score, a work and activities score, a retardation score, a agitation score, an anxiety psychic score, an anxiety somatic score, a somatic symptoms of gastrointestinal score, a general somatic score, a genital symptoms score, a hypochondriasis score, a loss of weight score, or a insight score. Subscores may comprise a numerical value of 0, 1, or 2, or a range of such numerical values. Subscores may comprise a numerical value of 0, 1, 2, 3, or 4, or a range of such numerical values. In some embodiments, the HDRS score comprises a numerical value below a threshold numerical value that is indicative of a depressive disorder. For example, a score of below 20 may indicate a lack of moderate or severe depression. In some embodiments, the subscore comprises a numerical value below a threshold numerical value that is indicative of the depressive disorder.

In some embodiments, the composition reduces the HDRS score relative to the baseline HDRS score. In some embodiments, the reduced HDRS score by observing and/or questioning the subject after administering the composition to the subject. In some embodiments, the HDRS score is decreased by about 2.5% or more, about 5% or more, or about 7.5% or more, relative to the baseline HDRS score. In some embodiments, the HDRS score is decreased by about 10% or more, relative to the baseline HDRS score. In some embodiments, the HDRS score is decreased by about 20% or more, about 30% or more, about 40% or more, about 50% or more, about 60% or more, about 70% or more, about 80% or more, about 90% or more, relative to the baseline HDRS score. In some embodiments, the HDRS score is decreased by no more than about 2.5%, no more than about 5%, or no more than about 7.5%, relative to the baseline HDRS score. In some embodiments, the HDRS score is decreased by no more than about 10%, relative to the baseline HDRS score. In some embodiments, the HDRS score is decreased by no more than about 20%, no more than about 30%, no more than about 40%, no more than about 50%, no more than about 60%, no more than about 70%, no more than about 80%, no more than about 90%, or no more than about 100% relative to the baseline HDRS score. In some embodiments, the HDRS score is decreased by 2.5%, 5%, 7.5%, 10%, 20%, 30%, 40%, 50%, 60%, 70%, 80%, 90%, or 100%, or by a range defined by any of the two aforementioned percentages. In some embodiments, the HDRS score is decreased by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 41, 42, 43, 44, 45, 46, 47, 48, 49, 50, 51, 52, 53, 54, 55 56, 57, 58, 59, or 60 points, relative to the baseline HDRS score, or by a range of points defined by any two of the aforementioned numbers of points relative to the baseline HDRS score. In some embodiments, the HDRS score is decreased by 1-5 points. In some embodiments, the HDRS score is decreased by 6-10 points. In some embodiments, the HDRS score is decreased by 11-15 points. In some embodiments, the HDRS score is decreased by 16-20 points. In some embodiments, the HDRS score is decreased by 21-25 points. In some embodiments, the HDRS score is decreased by 26-30 points. In some embodiments, the HDRS score is decreased by 31-35 points. In some embodiments, the HDRS score is decreased by 36-40 points. In some embodiments, the HDRS score is decreased by 41-45 points. In some embodiments, the HDRS score is decreased by 46-50 points. In some embodiments, the HDRS score is decreased by 51-55 points. In some embodiments, the HDRS score is decreased by 56-60 points.

In some embodiments, following treatment with the oligonucleotide, the HDRS score of the subject is decreased such that the HDRS score of the subject changes from severe depression to mild or moderate depression, or to normal non-depressed symptomology. In some embodiments, the HDRS score changes from moderate depression to mild depression, or to normal non-depressed symptomology. For example, the HDRS score of the subject may be below 20 following treatment. In some embodiments, the HDRS score changes from mild depression to normal non-depressed symptomology. For example, the HDRS score of the subject may be below 8 following treatment.

In some embodiments, the measurement is an anxiety measurement. The anxiety measurement may include an assessment of a symptom of anxiety. In some embodiments, the symptom of anxiety includes stress, worry, or restlessness. In some cases, the symptom of anxiety includes one or more behavioral symptoms such as hypervigilance, irritability, or restlessness. In some cases, the symptom of anxiety includes one or more cognitive symptoms such as lack of concentration, racing thoughts, or unwanted thoughts. In some cases, the symptom of anxiety includes one or more whole body symptoms such as fatigue or sweating. In some cases, the symptoms of anxiety include any of excessive worry, fear, feeling of impending doom, insomnia, nausea, palpitations, or trembling. In some embodiments, the symptom includes one or more panic attacks. The anxiety measurement may include a questionnaire or assessment. The assessment may include an amount, frequency, duration, or intensity of the anxiety or symptoms of anxiety. The anxiety measurement may include an amount of time since feeling anxious or since feeling symptoms of anxiety. The anxiety measurement may include a frequency of feeling anxious or feeling symptoms of anxiety. In some embodiments, the composition reduces the anxiety measurement relative to the baseline anxiety measurement. For example, the composition may reduce the anxiety measurement by 2.5%, 5%, 7.5%, 10%, 20%, 30%, 40%, 50%, 60%, 70%, 80%, 90%, or 100%, or by a range defined by any of the two aforementioned percentages.

In some embodiments, the measurement is an eating disorder measurement. Examples of eating disorders include anorexia, bulimia, binge eating disorder, pica, rumination, or avoidant eating disorder. In some embodiments, the eating disorder includes anorexia nervosa. In some embodiments, the eating disorder includes bulimia. In some embodiments, the eating disorder includes binge eating. In some embodiments, the eating disorder includes pica. The eating disorder measurement may include an assessment of a symptom of eating disorder. Some examples of symptoms of an eating disorder comprising anorexia nervosa include being considerably underweight compared with people of similar age and height, very restricted eating patterns, an intense fear of gaining weight or persistent behaviors to avoid gaining weight despite being underweight, a relentless pursuit of thinness and unwillingness to maintain a healthy weight, a heavy influence of body weight or perceived body shape on self-esteem, a distorted body image, or denial of being seriously underweight. The eating disorder measurement may include a questionnaire or assessment. The assessment may include an amount, frequency, duration, or intensity of the eating disorder or symptoms. The eating disorder measurement may include an amount of time since engaging in the eating disorder (e.g. binding, purging, or starving). The eating disorder measurement may include a frequency of engaging in the eating disorder. In some embodiments, the composition reduces the eating disorder measurement relative to the baseline eating disorder measurement. For example, the composition may reduce the eating disorder measurement by 2.5%, 5%, 7.5%, 10%, 20%, 30%, 40%, 50%, 60%, 70%, 80%, 90%, or 100%, or by a range defined by any of the two aforementioned percentages.

In some embodiments, the measurement is a substance-use measurement. In some embodiments, the substance-use measurement includes a determination of a level of addiction to an addictive substance. Examples of addictive substances include alcohol, antianxiety drugs, sedative drugs, caffeine, cannabis (e.g. including marijuana or synthetic cannabinoids), hallucinogens (e.g. LSD, phencyclidine, or psilocybin), inhalants (e.g. paint thinner or some glues), opioids (e.g. fentanyl, morphine, or oxycodone), stimulants (e.g. amphetamines or cocaine), tobacco, or anabolic steroids. The substance abuse measurement may include an amount of time since engaging in the substance-use disorder or experiencing symptoms of the substance-use disorder. The substance abuse measurement may include a frequency of engaging in the substance-use disorder or experiencing symptoms of the substance-use disorder. The determination of a level of addiction to an addictive substance may include a questionnaire or assessment. The substance abuse measurement or the assessment may include an amount, frequency, duration, or intensity of the substance-use disorder or symptoms. The determination of a level of addiction to an addictive substance may include an amount of time since ingesting the addictive substance. The determination of a level of addiction to an addictive substance may include a frequency of ingesting the addictive substance. In some embodiments, the composition reduces the substance abuse measurement relative to the baseline substance abuse measurement. For example, the composition may reduce the e substance abuse measurement by 2.5%, 5%, 7.5%, 10%, 20%, 30%, 40%, 50%, 60%, 70%, 80%, 90%, or 100%, or by a range defined by any of the two aforementioned percentages.

In some embodiments, the measurement is a PTSD measurement. In some embodiments, the PTSD measurement includes a determination of the level of severity of PTSD. The assessment of a sign or symptom of PTSD may include the number of signs or symptoms of PTSD. The determination of the level of severity of PTSD may include the time since last experiencing a PTSD flashback (e.g., reliving the traumatic event as if it were happening again), nightmare, or severe anxiety. The assessment may include a frequency in PTSD related flashbacks, nightmares, or severe anxiety episodes. The assessment may include a severity of a sign or symptom of PTSD. The assessment may include a frequency of a sign or symptom of PTSD. Exemplary signs and symptoms of PTSD may include intrusive memories (e.g., recurrent, unwanted distressing memories of a traumatic event, severe emotional distress or physical reactions to something that reminiscent of the traumatic event, attempts to avoid thinking or talking about the traumatic event, avoiding places, activities or people reminiscent of the traumatic event, thoughts of hopelessness, memory problems, difficulty maintaining close relationships, and feeling a lack of interest in activities that were once enjoyed. In some embodiments, the composition reduces the PTSD measurement relative to the baseline substance abuse measurement. For example, the composition may reduce the PTSD measurement by 2.5%, 5%, 7.5%, 10%, 20%, 30%, 40%, 50%, 60%, 70%, 80%, 90%, or 100%, or by a range defined by any of the two aforementioned percentages.

In some embodiments, the measurement is a bipolar disorder measurement. In some embodiments, the bipolar disorder measurement is a sign or symptom of bipolar disorder. The assessment of a sign or symptom of bipolar disorder may include a frequency of a sign or symptom of bipolar disorder. The assessment of a sign or symptom of bipolar disorder may include a severity of a sign or symptom of bipolar disorder. The assessment of a sign or symptom of bipolar disorder may include the number of signs or symptoms of bipolar disorder. Exemplary signs and symptoms of bipolar disorder include any of the bipolar signs and symptoms disclosed herein, including, manic episodes (e.g., experiencing feelings of increased activity, energy, or agitation, an exaggerated sense of well-being and self-confidence, a decreased need for sleep, racing thoughts, distractibility, and a decreased ability to control impulses), and major depressive episodes (e.g., experiencing a depressed mood, marked loss of interest of feelings of pleasure, fatigue or loss of energy, feelings of guilt or worthlessness, and a decreased ability to think or concentrate). In some embodiments, the composition reduces the bipolar disorder measurement relative to the baseline substance abuse measurement. For example, the composition may reduce the bipolar disorder measurement by 2.5%, 5%, 7.5%, 10%, 20%, 30%, 40%, 50%, 60%, 70%, 80%, 90%, or 100%, or by a range defined by any of the two aforementioned percentages.

In some embodiments, the measurement comprises a schizophrenia measurement. In some embodiments, the schizophrenia measurement is a sign or symptom of schizophrenia. The assessment of a sign or symptom of schizophrenia may include a frequency of a sign or symptom of schizophrenia. The assessment of a sign or symptom of schizophrenia may include a severity of a sign or symptom of schizophrenia. The assessment of a sign or symptom of schizophrenia may include the number of signs or symptoms of schizophrenia. Exemplary signs and symptoms of schizophrenia may include delusions, hallucinations, disorganized thoughts and speech, disorganized or abnormal motor behavior, and negative symptoms (e.g., social withdrawal, anhedonia, avolition, decreased sense of purpose, lack of interest in activities, flat affect, lack of eye contact, and physical inactivity. In some embodiments, the composition reduces the schizophrenia measurement relative to the baseline substance abuse measurement. For example, the composition may reduce the schizophrenia measurement by 2.5%, 5%, 7.5%, 10%, 20%, 30%, 40%, 50%, 60%, 70%, 80%, 90%, or 100%, or by a range defined by any of the two aforementioned percentages.

In some embodiments, the measurement comprises a psychosis measurement. In some embodiments, the psychosis measurement is a sign or symptom of psychosis. The assessment of a sign or symptom of psychosis may include a frequency of a sign or symptom of psychosis. The assessment of a sign or symptom of psychosis may include a severity of a sign or symptom of psychosis. The assessment of a sign or symptom of psychosis may include the number of signs or symptoms of psychosis. Exemplary signs and symptoms of psychosis may include difficulty concentrating, depressed mood, anxiety, excessive suspiciousness, delusions, and hallucinations. In some embodiments, the composition reduces the schizophrenia measurement relative to the baseline psychosis measurement. For example, the composition may reduce the psychosis measurement by 2.5%, 5%, 7.5%, 10%, 20%, 30%, 40%, 50%, 60%, 70%, 80%, 90%, or 100%, or by a range defined by any of the two aforementioned percentages.

In some embodiments, the measurement comprises a measurement of a neurological disorder. Non-limiting examples of measurements of neurological disorders include a measurement of cognitive function, a measurement of CNS amyloid plaque(s) (e.g., accumulation), a measurement of CNS tau accumulation, a measurement of CSF beta-amyloid 42 (e.g., accumulation), a measurement of CSF tau (e.g., accumulation), a measurement of CSF phospho-tau (e.g., accumulation), a measurement of Lewy bodies (e.g., accumulation), or a measurement of CSF alpha-synuclein (e.g., accumulation). Further non-limiting examples of measurements include a measurement of headache signs and/or symptoms, a measurement of migraine symptoms and/or signs, a measurement of chronic pain symptoms and/or signs, a measurement of fibromyalgia symptoms and/or signs, a measurement of chronic fatigue syndrome (ME) symptoms and/or signs, and a measurement of motor neuron disease (e.g., ALS) symptoms and/or signs. In some embodiments, the composition reduces the neurological disorder measurement relative to the baseline neurological disorder measurement. For example, the composition may reduce the neurological disorder measurement by 2.5%, 5%, 7.5%, 10%, 20%, 30%, 40%, 50%, 60%, 70%, 80%, 90%, or 100%, or by a range defined by any of the two aforementioned percentages.

In some embodiments, the measurement is a cognitive function measurement. The cognitive function measurement may be obtained directly from the subject. For example, the subject may be administered a test. The test may include a cognitive test such as the Montreal Cognitive Assessment (MoCA), Mini-Mental State Exam (MMSE), or Mini-Cog. The test may include assessment of basic cognitive functions such as memory, language, executive frontal lobe function, apraxia, visuospatial ability, behavior, mood, orientation, or attention. The cognitive function measurement may include a score. The cognitive function measurement may be indicative of a lack of cognitive impairment. In some embodiments, the cognitive function measurement is indicative of mild cognitive impairment, and the baseline cognitive function measurement is indicative of severe cognitive impairment. The cognitive function measurement may be indicative of a neurological disorder.

In some embodiments, the composition increases the cognitive function measurement relative to the baseline cognitive function measurement. In some embodiments, the increase is measured directly in the subject after administering the composition to the subject. In some embodiments, the cognitive function measurement is increased by about 2.5% or more, about 5% or more, or about 7.5% or more, relative to the baseline cognitive function measurement. In some embodiments, the cognitive function measurement is increased by about 10% or more, relative to the baseline cognitive function measurement. In some embodiments, the cognitive function measurement is increased by about 20% or more, about 30% or more, about 40% or more, about 50% or more, about 60% or more, about 70% or more, about 80% or more, about 90% or more, relative to the baseline cognitive function measurement. In some embodiments, the cognitive function measurement is increased by about 100% or more, increased by about 250% or more, increased by about 500% or more, increased by about 750% or more, or increased by about 1000% or more, relative to the baseline cognitive function measurement. In some embodiments, the cognitive function measurement is increased by no more than about 2.5%, no more than about 5%, or no more than about 7.5%, relative to the baseline cognitive function measurement. In some embodiments, the cognitive function measurement is increased by no more than about 10%, relative to the baseline cognitive function measurement. In some embodiments, the cognitive function measurement is increased by no more than about 20%, no more than about 30%, no more than about 40%, no more than about 50%, no more than about 60%, no more than about 70%, no more than about 80%, no more than about 90%, or no more than about 100% relative to the baseline cognitive function measurement. In some embodiments, the cognitive function measurement is increased by no more than about 100%, increased by no more than about 250%, increased by no more than about 500%, increased by no more than about⁷50%, or increased by no more than about 1000%, relative to the baseline cognitive function measurement. In some embodiments, the cognitive function measurement is increased by 2.5%, 5%, 7.5%, 10%, 20%, 30%, 40%, 50%, 60%, 70%, 80%, 90%, 100%, 250%, 500%, 750%, or 1000%, or by a range defined by any of the two aforementioned percentages.

In some embodiments, the measurement is an amyloid plaque measurement. The amyloid plaque measurement may include a central nervous system (CNS) amyloid plaque measurement. In some embodiments, the amyloid plaque measurement includes a concentration or amount. The amyloid plaque measurement may be performed using an imaging device. The imaging device may include a positron emission tomography (PET) device. The amyloid plaque measurement may be performed on a biopsy. The amyloid plaque measurement may be performed using a spinal tap (for example, when the amyloid plaque measurement includes a cerebrospinal fluid (CSF) amyloid plaque measurement). In some embodiments, the amyloid plaque measurement is obtained by an assay such as an immunoassay. The beta amyloid plaque measurement may be indicative of a treatment effect of the oligonucleotide on a neurodegenerative disease such as Alzheimer's disease.

In some embodiments, the composition reduces the amyloid plaque measurement relative to the baseline amyloid plaque measurement. In some embodiments, the reduction is measured in a second sample obtained from the subject after administering the composition to the subject. In some embodiments, the reduction is measured directly in the subject after administering the composition to the subject. In some embodiments, the amyloid plaque measurement is decreased by about 2.5% or more, about 5% or more, or about 7.5% or more, relative to the baseline amyloid plaque measurement. In some embodiments, the amyloid plaque measurement is decreased by about 10% or more, relative to the baseline amyloid plaque measurement. In some embodiments, the amyloid plaque measurement is decreased by about 20% or more, about 30% or more, about 40% or more, about 50% or more, about 60% or more, about 70% or more, about 80% or more, about 90% or more, relative to the baseline amyloid plaque measurement. In some embodiments, the amyloid plaque measurement is decreased by no more than about 2.5%, no more than about 5%, or no more than about 7.5%, relative to the baseline amyloid plaque measurement. In some embodiments, the amyloid plaque measurement is decreased by no more than about 10%, relative to the baseline amyloid plaque measurement. In some embodiments, the amyloid plaque measurement is decreased by no more than about 20%, no more than about 30%, no more than about 40%, no more than about 50%, no more than about 60%, no more than about 70%, no more than about 80%, no more than about 90%, or no more than about 100% relative to the baseline amyloid plaque measurement. In some embodiments, the amyloid plaque measurement is decreased by 2.5%, 5%, 7.5%, 10%, 20%, 30%, 40%, 50%, 60%, 70%, 80%, 90%, or 100%, or by a range defined by any of the two aforementioned percentages.

In some embodiments, the measurement is a tau measurement. In some embodiments, the tau measurement includes a concentration or amount. The tau measurement may be performed on a biopsy. In some embodiments, the tau measurement is obtained by an assay such as an immunoassay. The beta tau measurement may be indicative of a treatment effect of the oligonucleotide on a neurodegenerative disease such as Alzheimer's disease or Parkinson's disease.

In some embodiments, the tau measurement is a central nervous system (CNS) tau measurement. The tau measurement may include a total tau measurement. The tau measurement may include a unphosphorylated tau measurement. The tau measurement may include a phosphorylated tau (phospho-tau) measurement. In some embodiments, the tau measurement is a tau accumulation measurement. In some embodiments, the tau measurement is a CNS tau accumulation measurement. The CNS tau accumulation measurement may be indicative of a treatment effect of the oligonucleotide on a neurodegenerative disease such as Alzheimer's disease or Parkinson's disease.

In some embodiments, the composition reduces the CNS tau accumulation measurement relative to the baseline CNS tau accumulation measurement. In some embodiments, the reduction is measured in a second sample obtained from the subject after administering the composition to the subject. In some embodiments, the CNS tau accumulation measurement is decreased by about 2.5% or more, about 5% or more, or about 7.5% or more, relative to the baseline CNS tau accumulation measurement. In some embodiments, the CNS tau accumulation measurement is decreased by about 10% or more, relative to the baseline CNS tau accumulation measurement. In some embodiments, the CNS tau accumulation measurement is decreased by about 20% or more, about 30% or more, about 40% or more, about 50% or more, about 60% or more, about 70% or more, about 80% or more, about 90% or more, relative to the baseline CNS tau accumulation measurement. In some embodiments, the CNS tau accumulation measurement is decreased by no more than about 2.5%, no more than about 5%, or no more than about 7.5%, relative to the baseline CNS tau accumulation measurement. In some embodiments, the CNS tau accumulation measurement is decreased by no more than about 10%, relative to the baseline CNS tau accumulation measurement. In some embodiments, the CNS tau accumulation measurement is decreased by no more than about 20%, no more than about 30%, no more than about 40%, no more than about 50%, no more than about 60%, no more than about 70%, no more than about 80%, no more than about 90%, or no more than about 100% relative to the baseline CNS tau accumulation measurement. In some embodiments, the CNS tau accumulation measurement is decreased by 2.5%, 5%, 7.5%, 10%, 20%, 30%, 40%, 50%, 60%, 70%, 80%, 90%, or 100%, or by a range defined by any of the two aforementioned percentages.

The tau measurement may include a cerebrospinal fluid (CSF) tau measurement. The CSF tau measurement may be performed after use of a spinal tap. The CSF tau measurement may be indicative of a treatment effect of the oligonucleotide on a neurodegenerative disease such as Alzheimer's disease or Parkinson's disease.

In some embodiments, the composition reduces the CSF tau measurement relative to the baseline CSF tau measurement. In some embodiments, the reduction is measured in a second sample obtained from the subject after administering the composition to the subject. In some embodiments, the reduction is measured in a second CSF sample obtained from the subject after administering the composition to the subject. In some embodiments, the CSF tau measurement is decreased by about 2.5% or more, about 5% or more, or about 7.5% or more, relative to the baseline CSF tau measurement. In some embodiments, the CSF tau measurement is decreased by about 10% or more, relative to the baseline CSF tau measurement. In some embodiments, the CSF tau measurement is decreased by about 20% or more, about 30% or more, about 40% or more, about 50% or more, about 60% or more, about 70% or more, about 80% or more, about 90% or more, relative to the baseline CSF tau measurement. In some embodiments, the CSF tau measurement is decreased by no more than about 2.5%, no more than about 5%, or no more than about 7.5%, relative to the baseline CSF tau measurement. In some embodiments, the CSF tau measurement is decreased by no more than about 10%, relative to the baseline CSF tau measurement. In some embodiments, the CSF tau measurement is decreased by no more than about 20%, no more than about 30%, no more than about 40%, no more than about 50%, no more than about 60%, no more than about 70%, no more than about 80%, no more than about 90%, or no more than about 100% relative to the baseline CSF tau measurement. In some embodiments, the CSF tau measurement is decreased by 2.5%, 5%, 7.5%, 10%, 20%, 30%, 40%, 50%, 60%, 70%, 80%, 90%, or 100%, or by a range defined by any of the two aforementioned percentages.

The CSF tau measurement may include a CSF phospho-tau measurement. The CSF phospho-tau measurement may include an amount of phospho-tau in relation to total tau or unphosphorylated tau. For example, the CSF phospho-tau measurement may include a phospho-tau/tau ratio. The CSF phospho-tau measurement may be indicative of a treatment effect of the oligonucleotide on a neurodegenerative disease such as Alzheimer's disease or Parkinson's disease.

In some embodiments, the composition reduces the CSF phospho-tau measurement relative to the baseline CSF phospho-tau measurement. In some embodiments, the reduction is measured in a second sample obtained from the subject after administering the composition to the subject. In some embodiments, the reduction is measured in a second CSF sample obtained from the subject after administering the composition to the subject. In some embodiments, the CSF phospho-tau measurement is decreased by about 2.5% or more, about 5% or more, or about 7.5% or more, relative to the baseline CSF phospho-tau measurement. In some embodiments, the CSF phospho-tau measurement is decreased by about 10% or more, relative to the baseline CSF phospho-tau measurement. In some embodiments, the CSF phospho-tau measurement is decreased by about 20% or more, about 30% or more, about 40% or more, about 50% or more, about 60% or more, about 70% or more, about 80% or more, about 90% or more, relative to the baseline CSF phospho-tau measurement. In some embodiments, the CSF phospho-tau measurement is decreased by no more than about 2.5%, no more than about 5%, or no more than about 7.5%, relative to the baseline CSF phospho-tau measurement. In some embodiments, the CSF phospho-tau measurement is decreased by no more than about 10%, relative to the baseline CSF phospho-tau measurement. In some embodiments, the CSF phospho-tau measurement is decreased by no more than about 20%, no more than about 30%, no more than about 40%, no more than about 50%, no more than about 60%, no more than about 70%, no more than about 80%, no more than about 90%, or no more than about 100% relative to the baseline CSF phospho-tau measurement. In some embodiments, the CSF phospho-tau measurement is decreased by 2.5%, 5%, 7.5%, 10%, 20%, 30%, 40%, 50%, 60%, 70%, 80%, 90%, or 100%, or by a range defined by any of the two aforementioned percentages.

In some embodiments, the measurement is a alpha-synuclein measurement. The alpha-synuclein measurement may include a cerebrospinal fluid (CSF) alpha-synuclein measurement. In some embodiments, the alpha-synuclein measurement includes a concentration or amount. The alpha-synuclein measurement may be performed on a biopsy. The alpha-synuclein measurement may be performed using a spinal tap (for example, when the alpha-synuclein measurement includes a CSF alpha-synuclein measurement). In some embodiments, the alpha-synuclein measurement is obtained by an assay such as an immunoassay. The alpha-synuclein measurement may be indicative of a treatment effect of the oligonucleotide on a neurodegenerative disease such as Parkinson's disease. The alpha-synuclein measurement may be indicative of a treatment effect of the oligonucleotide on dementia.

In some embodiments, the composition reduces the alpha-synuclein measurement relative to the baseline alpha-synuclein measurement. In some embodiments, the reduction is measured in a second sample obtained from the subject after administering the composition to the subject. In some embodiments, the alpha-synuclein measurement is decreased by about 2.5% or more, about 5% or more, or about 7.5% or more, relative to the baseline alpha-synuclein measurement. In some embodiments, the alpha-synuclein measurement is decreased by about 10% or more, relative to the baseline alpha-synuclein measurement. In some embodiments, the alpha-synuclein measurement is decreased by about 20% or more, about 30% or more, about 40% or more, about 50% or more, about 60% or more, about 70% or more, about 80% or more, about 90% or more, relative to the baseline alpha-synuclein measurement. In some embodiments, the alpha-synuclein measurement is decreased by no more than about 2.5%, no more than about 5%, or no more than about 7.5%, relative to the baseline alpha-synuclein measurement. In some embodiments, the alpha-synuclein measurement is decreased by no more than about 10%, relative to the baseline alpha-synuclein measurement. In some embodiments, the alpha-synuclein measurement is decreased by no more than about 20%, no more than about 30%, no more than about 40%, no more than about 50%, no more than about 60%, no more than about 70%, no more than about 80%, no more than about 90%, or no more than about 100% relative to the baseline alpha-synuclein measurement. In some embodiments, the alpha-synuclein measurement is decreased by 2.5%, 5%, 7.5%, 10%, 20%, 30%, 40%, 50%, 60%, 70%, 80%, 90%, or 100%, or by a range defined by any of the two aforementioned percentages.

In some embodiments, the measurement is a Lewy body measurement. The Lewy body measurement may include a central nervous system (CNS) Lewy body measurement. In some embodiments, the Lewy body measurement includes a concentration or amount. The Lewy body measurement may be performed using an imaging device. The imaging device may include a positron emission tomography (PET) device. The beta Lewy body measurement may be indicative of a treatment effect of the oligonucleotide on dementia.

In some embodiments, the composition reduces the Lewy body measurement relative to the baseline Lewy body measurement. In some embodiments, the reduction is measured in a second sample obtained from the subject after administering the composition to the subject. In some embodiments, the reduction is measured directly in the subject after administering the composition to the subject. In some embodiments, the Lewy body measurement is decreased by about 2.5% or more, about 5% or more, or about 7.5% or more, relative to the baseline Lewy body measurement. In some embodiments, the Lewy body measurement is decreased by about 10% or more, relative to the baseline Lewy body measurement. In some embodiments, the Lewy body measurement is decreased by about 20% or more, about 30% or more, about 40% or more, about 50% or more, about 60% or more, about 70% or more, about 80% or more, about 90% or more, relative to the baseline Lewy body measurement. In some embodiments, the Lewy body measurement is decreased by no more than about 2.5%, no more than about 5%, or no more than about 7.5%, relative to the baseline Lewy body measurement. In some embodiments, the Lewy body measurement is decreased by no more than about 10%, relative to the baseline Lewy body measurement. In some embodiments, the Lewy body measurement is decreased by no more than about 20%, no more than about 30%, no more than about 40%, no more than about 50%, no more than about 60%, no more than about 70%, no more than about 80%, no more than about 90%, or no more than about 100% relative to the baseline Lewy body measurement. In some embodiments, the Lewy body measurement is decreased by 2.5%, 5%, 7.5%, 10%, 20%, 30%, 40%, 0%, 60%, 70%, 80%, 90%, or 100%, or by a range defined by any of the two aforementioned percentages.

In some embodiments, the measurement is a beta-amyloid 42 measurement. The beta-amyloid 42 measurement may include a cerebrospinal fluid (CSF) beta-amyloid 42 measurement. In some embodiments, the beta-amyloid 42 measurement includes a concentration or amount. The beta-amyloid 42 measurement may be performed on a biopsy. The beta-amyloid 42 measurement may be performed using a spinal tap (for example, when the beta-amyloid 42 measurement includes a CSF beta-amyloid 42 measurement). In some embodiments, the beta-amyloid 42 measurement is obtained by an assay such as an immunoassay. The beta-amyloid 42 measurement may be indicative of a treatment effect of the oligonucleotide on a neurodegenerative disease such as Alzheimer's disease.

In some embodiments, the composition reduces the CSF beta-amyloid 42 measurement relative to the baseline beta-amyloid 42 measurement. In some embodiments, the reduction is measured in a second sample (for example, a CSF sample) obtained from the subject after administering the composition to the subject. In some embodiments, the CSF beta-amyloid 42 measurement is decreased by about 2.5% or more, about 5% or more, or about 7.5% or more, relative to the baseline CSF beta-amyloid 42 measurement. In some embodiments, the CSF beta-amyloid 42 measurement is decreased by about 10% or more, relative to the baseline CSF beta-amyloid 42 measurement. In some embodiments, the CSF beta-amyloid 42 measurement is decreased by about 20% or more, about 30% or more, about 40% or more, about 50% or more, about 60% or more, about 70% or more, about 80% or more, about 90% or more, relative to the baseline CSF beta-amyloid 42 measurement. In some embodiments, the CSF beta-amyloid 42 measurement is decreased by no more than about 2.5%, no more than about 5%, or no more than about 7.5%, relative to the baseline CSF beta-amyloid 42 measurement. In some embodiments, the CSF beta-amyloid 42 measurement is decreased by no more than about 10%, relative to the baseline CSF beta-amyloid 42 measurement. In some embodiments, the CSF beta-amyloid 42 measurement is decreased by no more than about 20%, no more than about 30%, no more than about 40%, no more than about 50%, no more than about 60%, no more than about 70%, no more than about 80%, no more than about 90%, or no more than about 100% relative to the baseline CSF beta-amyloid 42 measurement. In some embodiments, the CSF beta-amyloid 42 measurement is decreased by 2.5%, 5%, 7.5%, 10%, 20%, 30%, 40%, 50%, 60%, 70%, 80%, 90%, or 100%, or by a range defined by any of the two aforementioned percentages.

In some embodiments, the measurement is a headache measurement. some embodiments, the headache measurement is a headache sign or symptom measurement. In some embodiments, the headache measurement is a migraine (e.g., with aura or without aura) measurement. In some embodiments, the headache measurement is a frequency of a headache sign or symptom measurement. In some embodiments, the headache measurement is a severity of a headache sign or symptom measurement. In some embodiments, the headache measurement is a number of headache signs or symptoms. Exemplary signs and symptoms of headaches include pain (e.g., deep and constant) in the cheekbones, forehead, bridge of the nose, the cranium, or the back of the neck, aura, photophobia, phonophobia, and emesis. In some embodiments, the composition reduces the headache measurement relative to the baseline headache measurement. For example, the composition may reduce the headache measurement by 2.5%, 5%, 7.5%, 10%, 20%, 30%, 40%, 50%, 60%, 70%, 80%, 90%, or 100%, or by a range defined by any of the two aforementioned percentages.

In some embodiments, the measurement is a chronic pain measurement. In some embodiments, chronic pain measurement is a fibromyalgia measurement. In some embodiments, the chronic pain measurement is a chronic pain sign or symptom measurement. In some embodiments, the chronic pain measurement is a frequency of a chronic pain sign or symptom measurement. In some embodiments, the chronic pain measurement is a severity of a chronic pain sign or symptom measurement. In some embodiments, the chronic pain measurement is a number of chronic pain signs or symptoms. Exemplary signs and symptoms of fibromyalgia include muscular pain, fatigues, depression, anxiety, sleeplessness, headache, and difficulty concentrating. Exemplary chronic pain disorders include postsurgical pain, post-trauma pain, low back pain, cancer pain, arthritis pain, muscular pain, and neuropathic pain (e.g., diabetic neuropathy). In some embodiments, the composition reduces the chronic pain (e.g., fibromyalgia) measurement relative to the baseline chronic pain (e.g., fibromyalgia) measurement. For example, the composition may reduce the chronic pain (e.g., fibromyalgia) measurement by 2.5%, 5%, 7.5%, 10%, 20%, 30%, 40%, 50%, 60%, 70%, 80%, 90%, or 100%, or by a range defined by any of the two aforementioned percentages.

In some embodiments, the measurement is a chronic fatigue syndrome (also referred to as myalgic encephalomyelitis) measurement. In some embodiments, the chronic fatigue syndrome measurement is a chronic fatigue syndrome sign or symptom measurement. In some embodiments, the chronic fatigue syndrome measurement is a frequency of a headache sign or symptom measurement. In some embodiments, the chronic fatigue syndrome measurement is a severity of a chronic fatigue syndrome sign or symptom measurement. In some embodiments, the chronic fatigue syndrome n measurement is a number of chronic fatigue syndrome signs or symptoms. Exemplary signs and symptoms of chronic fatigue syndrome include extreme fatigue that lasts for extended periods of time (e.g., for at least six months) that cannot be fully explained by an underlying medical condition, fatigue that worsens with physical or mental activity, pain (e.g., joint or muscular), malaise, forgetfulness, anxiety, and depression. In some embodiments, the composition reduces the chronic fatigue syndrome measurement relative to the baseline chronic fatigue syndrome measurement. For example, the composition may reduce the chronic fatigue syndrome measurement by 2.5%, 5%, 7.5%, 10%, 20%, 30%, 40%, 50%, 60%, 70%, 80%, 90%, or 100%, or by a range defined by any of the two aforementioned percentages.

In some embodiments, the measurement is a motor neuron disease measurement. In some embodiments, the motor neuron disease measurement is an amyotrophic lateral sclerosis (ALS) measurement. In some embodiments, the motor neuron disease measurement is a motor neuron disease sign or symptom measurement. In some embodiments, the motor neuron disease measurement is a frequency of a motor neuron disease sign or symptom measurement. In some embodiments, the motor neuron disease measurement is a severity of a motor neuron disease sign or symptom measurement. In some embodiments, the motor neuron disease measurement is a number of motor neuron disease signs or symptoms. Exemplary forms of motor neuron diseases include progressive bulbar palsy (PBP), progressive muscular atrophy (PMA), ALS, and primary lateral sclerosis (PLS). Exemplary signs and symptoms of motor neuron diseases include motor control difficulties (e.g., difficulty walking or completing normal daily activities), muscular weakness, slurred speech, difficulty swallowing, and muscle cramps and twitching (e.g., in the arms, shoulders, or tongue). In some embodiments, the composition reduces the motor neuron disease (e.g., ALS) measurement relative to the baseline chronic fatigue syndrome measurement. For example, the composition may reduce the motor neuron disease (e.g., ALS) measurement by 2.5%, 5%, 7.5%, 10%, 20%, 30%, 40%, 50%, 60%, 70%, 80%, 90%, or 100%, or by a range defined by any of the two aforementioned percentages.

In some embodiments, the measurement is a fibrinogen measurement. In some embodiments, the measurement is a measurement of circulating fibrinogen. In some embodiments, the composition reduces the fibrinogen measurement relative to the baseline fibrinogen measurement. In some embodiments, the composition reduces the circulating fibrinogen measurement relative to the baseline circulating fibrinogen measurement. In some embodiments, the fibrinogen measurement is decreased by about 2.5% or more, about 5% or more, or about 7.5% or more, relative to the baseline fibrinogen measurement. In some embodiments, the fibrinogen measurement is decreased by about 10% or more, relative to the baseline fibrinogen measurement. In some embodiments, the fibrinogen measurement is decreased by about 20% or more, about 30% or more, about 40% or more, about 50% or more, about 60% or more, about 70% or more, about 80% or more, about 90% or more, or about 100%, relative to the baseline fibrinogen measurement. In some embodiments, the fibrinogen measurement is decreased by no more than about 2.5%, no more than about 5%, or no more than about 7.5%, relative to the baseline fibrinogen measurement. In some embodiments, the fibrinogen measurement is decreased by no more than about 10%, relative to the baseline fibrinogen measurement. In some embodiments, the fibrinogen measurement is decreased by no more than about 20%, no more than about 30%, no more than about 40%, no more than about 50%, no more than about 60%, no more than about 70%, no more than about 80%, no more than about 90%, or no more than about 100% relative to the baseline fibrinogen measurement. In some embodiments, the fibrinogen measurement is decreased by 2.5%, 5%, 7.5%, 19%, 20%, 30%, 40%, 50%, 60%, 70%, 80%, 90%, or 100%, or by a range defined by any of the two aforementioned percentages.

In some embodiments, the measurement is a clotting or coagulation measurement. In some embodiments, the clotting or coagulation measurement is a prothrombin time (PT). In some embodiments, the clotting or coagulation measurement is an International Normalized Ratio (INR). In some embodiments, the clotting or coagulation measurement is an activated partial thromboplastin time (aPTT). In some embodiments, the composition reduces the clotting or coagulation measurement relative to the baseline clotting or coagulation measurement. In some embodiments, the clotting or coagulation measurement is increased by about 2.5% or more, about 5% or more, or about 7.5% or more, relative to the baseline clotting or coagulation measurement. In some embodiments, the clotting or coagulation measurement is increased by about 10% or more, relative to the baseline clotting or coagulation measurement. In some embodiments, the clotting or coagulation measurement is increased by about 20% or more, about 30% or more, about 40% or more, about 50% or more, about 60% or more, about 70% or more, about 80% or more, about 90% or more, or about 100%, relative to the baseline clotting or coagulation measurement. In some embodiments, the clotting or coagulation measurement is increased by no more than about 2.5%, no more than about 5%, or no more than about 7.5%, relative to the baseline clotting or coagulation measurement. In some embodiments, the clotting or coagulation measurement is increased by no more than about 10%, relative to the baseline clotting or coagulation measurement. In some embodiments, the clotting or coagulation measurement is increased by no more than about 20%, no more than about 30%, no more than about 40%, no more than about 50%, no more than about 60%, no more than about 70%, no more than about 80%, no more than about 90%, or no more than about 100% relative to the baseline clotting or coagulation measurement. In some embodiments, the clotting or coagulation measurement is increased by 2.5%, 5%, 7.5%, 15%, 20%, 30%, 40%, 50%, 60%, 70%, 80%, 90%, or 100%, or by a range defined by any of the two aforementioned percentages. In some embodiments, the clotting or coagulation measurement is increased be no more than about 20%, no more than about 40%, no more than about 80%, no more than about 100%, no more than about 120%, no more than about 140%, no more than about 160%, no more than about 180%, no more than about 200%, no more than about 300%, no more than about 400%, no more than about 500%, no more than about 600%, no more than about 700%<no more than about 800%, no more than about 900%, or more than about 1000% relative to the baseline clotting or coagulation measurement.

In some embodiments, the measurement is an FGG protein measurement. In some embodiments, the FGG protein measurement comprises an FGG protein level. In some embodiments, the FGG protein level is a FGG protein level in the whole body. In some embodiments, the FGG protein level is a FGG protein level in the blood. In some embodiments, the FGG protein level is a FGG protein level in the brain. In some embodiments, the FGG protein level is a FGG protein level in the liver. In some embodiments, the FGG protein level is indicated as a mass or percentage of FGG protein per sample weight. In some embodiments, the FGG protein level is indicated as a mass or percentage of FGG protein per sample volume. In some embodiments, the FGG protein level is indicated as a mass or percentage of FGG protein per total protein within the sample. In some embodiments, the FGG protein measurement is a circulating FGG protein measurement. In some embodiments, the FGG protein measurement is obtained by an assay such as an immunoassay, a colorimetric assay, or a fluorescence assay.

In some embodiments, the composition reduces the FGG protein measurement relative to the baseline FGG protein measurement. In some embodiments, the composition reduces circulating FGG protein levels relative to the baseline FGG protein measurement. In some embodiments, the composition reduces tissue (e.g. brain, liver, blood, or whole body) FGG protein levels relative to the baseline FGG protein measurement. In some embodiments, the reduced FGG protein levels are measured in a second sample obtained from the subject after administering the composition to the subject. In some embodiments, the FGG protein measurement is decreased by about 2.5% or more, about 5% or more, or about 7.5% or more, relative to the baseline FGG protein measurement. In some embodiments, the FGG protein measurement is decreased by about 10% or more, relative to the baseline FGG protein measurement. In some embodiments, the FGG protein measurement is decreased by about 20% or more, about 30% or more, about 40% or more, about 50% or more, about 60% or more, about 70% or more, about 80% or more, about 90% or more, or about 100%, relative to the baseline FGG protein measurement. In some embodiments, the FGG protein measurement is decreased by no more than about 2.5%, no more than about 5%, or no more than about 7.5%, relative to the baseline FGG protein measurement. In some embodiments, the FGG protein measurement is decreased by no more than about 10%, relative to the baseline FGG protein measurement. In some embodiments, the FGG protein measurement is decreased by no more than about 20%, no more than about 30%, no more than about 40%, no more than about 50%, no more than about 60%, no more than about 70%, no more than about 80%, no more than about 90%, or no more than about 100% relative to the baseline FGG protein measurement. In some embodiments, the FGG protein measurement is decreased by 2.5%, 5%, 7.5%, 19%, 20%, 30%, 40%, 50%, 60%, 70%, 80%, 90%, or 100%, or by a range defined by any of the two aforementioned percentages.

In some embodiments, the measurement is an FGG mRNA measurement. In some embodiments, the FGG mRNA measurement comprises an FGG mRNA level. In some embodiments, the FGG mRNA level is measured in the liver. In some embodiments, the FGG mRNA level is indicated as an amount or percentage of FGG mRNA per sample weight. In some embodiments, the FGG mRNA level is indicated as an amount or percentage of FGG mRNA per sample volume. In some embodiments, the FGG mRNA level is indicated as an amount or percentage of FGG mRNA per total mRNA within the sample. In some embodiments, the FGG mRNA level is indicated as an amount or percentage of FGG mRNA per total nucleic acids within the sample. In some embodiments, the FGG mRNA level is indicated relative to another mRNA level, such as an mRNA level of a housekeeping gene, within the sample. In some embodiments, the FGG mRNA measurement is obtained by an assay such as a PCR assay. In some embodiments, the PCR comprises qPCR. In some embodiments, the PCR comprises reverse transcription of the FGG mRNA.

In some embodiments, the composition reduces the FGG mRNA measurement relative to the baseline FGG mRNA measurement. In some embodiments, the FGG mRNA measurement is obtained in a second sample obtained from the subject after administering the composition to the subject. In some embodiments, the composition reduces FGG mRNA levels relative to the baseline FGG mRNA levels. In some embodiments, the reduced FGG mRNA levels are measured in a second sample obtained from the subject after administering the composition to the subject. In some embodiments, the second sample is a liver sample. In some embodiments, the FGG mRNA measurement is reduced by about 2.5% or more, about 5% or more, or about 7.5% or more, relative to the baseline v mRNA measurement. In some embodiments, the FGG mRNA measurement is decreased by about 10% or more, relative to the baseline FGG mRNA measurement. In some embodiments, the FGG mRNA measurement is decreased by about 20% or more, about 30% or more, about 40% or more, about 50% or more, about 60% or more, about 70% or more, about 80% or more, about 90% or more, or about 100%, relative to the baseline FGG mRNA measurement. In some embodiments, the FGG mRNA measurement is decreased by no more than about 2.5%, no more than about 5%, or no more than about 7.5%, relative to the baseline FGG mRNA measurement. In some embodiments, the FGG mRNA measurement is decreased by no more than about 10%, relative to the baseline FGG mRNA measurement. In some embodiments, the FGG mRNA measurement is decreased by no more than about 20%, no more than about 30%, no more than about 40%, no more than about 50%, no more than about 60%, no more than about 70%, no more than about 80%, no more than about 90%, or no more than about 100%, relative to the baseline FGG mRNA measurement. In some embodiments, the FGG mRNA measurement is decreased by 2.5%, 5%, 7.5%, 10%, 20%, 30%, 40%, 50%, 60%, 70%, 80%, 90%, or 100% or by a range defined by any of the two aforementioned percentages.

### III. Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

The terms “subject,” and “patient” may be used interchangeably herein. A “subject” can be a biological entity containing expressed genetic materials. The biological entity can be a plant, animal, or microorganism, including, for example, bacteria, viruses, fungi, and protozoa. The subject can be a mammal. The mammal can be a human. The subject may be diagnosed or suspected of being at high risk for a disease. In some cases, the subject is not necessarily diagnosed or suspected of being at high risk for the disease.

As used herein, the term “about” a number refers to that number plus or minus 10% of that number. The term “about” a range refers to that range minus 10% of its lowest value and plus 10% of its greatest value.

As used herein, the terms “treatment” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the symptoms of a disease may undergo treatment, even though a diagnosis of this disease may not have been made.

Some embodiments refer to nucleic acid sequence information. It is contemplated that in some embodiments, thymine (T) may be interchanged with uracil (U), or vice versa. For example, some sequences in the sequence listing may recite Ts, but these may be replaced with Us in some embodiments. In some oligonucleotides with nucleic acid sequences that include uracil, the uracil may be replaced with thymine. Similarly, in some oligonucleotides with nucleic acid sequences that include thymine, the thymine may be replaced with uracil. In some embodiments, an oligonucleotide such as an siRNA comprises or consists of RNA. In some embodiments, the oligonucleotide may comprise or consist of DNA. For example, an ASO may include DNA.

The section headings used herein are for organizational purposes only and are not to be construed as limiting the subject matter described.

### VI. Examples

### Example 1: Functional Variants in FGG Demonstrate Protective Associations for Psychiatric and Neurodegenerative Diseases

Variants in FGG were evaluated for associations with psychiatric diseases and neurological diseases, and related traits in approximately 382,000 individuals with genotype data from the UK Biobank cohort. Variants evaluated included: (1) rs148685782, a rare (AAF=0.004) FGG missense variant (Ala108Gly; A108G), which has been experimentally characterized as a FGG ↓ pQTL and (2) rs6063, a rare (AAF=0.005) FGG missense variant (Gly191Arg; G191R), which may have a deleterious impact on the FGG protein. Both variants were considered hypomorphic or loss-of-function variants that result in a decrease in the abundance or activity of the FGG gene product. Also evaluated was an FGG gene burden test which aggregated rs148685782, rs6063 and several additional rare nonsynonymous variants in FGG.

The analyses presented used a logistic or linear regression model with age, sex and the first ten principal components of genetic ancestry as covariates. The analyses resulted in identification of associations for the individual FGG variants and the FGG gene burden (Tables 1A-1C and 2A-2B).

TABLE 1A
FGG psychiatric disease associations

| | | | | Major Depressive | |
|---|---|---|---|---|---|
| | | | | Disorder (n = 35,446) | |
| Variant | Gene | Function | AAF | P value | OR |
| rs148685782 | FGG | Missense A108G; | 0.004 | 0.004 | ↓0.861 |
| | | FGG IpQTL | | | |
| rs6063 | FGG | Missense G191R | 0.005 | 0.003 | ↓0.848 |
| Gene Burden | FGG | Aggregation of rare | 0.009 | 6.45E−06 | ↓0.857 |
| | | deleterious variants | | | |

TABLE 1B
FGG psychiatric disease associations

| | |
|---|---|
| SSRI Medication | Intentional Self Harm |
| (n = 40,721) | (n = 2,395) |

| Variant | P value | OR | P value | OR |
|---|---|---|---|---|
| rs148685782 | 0.01 | ↓0.906 | 8.14E−04 | ↓0.359 |
| rs6063 | 0.015 | ↓0.917 | 0.201 | ↓0.760 |
| Gene Burden | 1.64E−04 | ↓0.907 | 0.002 | ↓0.575 |

TABLE 1C
FGG psychiatric disease associations

| | Family History of Severe | | Post-Traumatic Stress | |
|---|---|---|---|---|
| | Depression (n = 49,923) | | Disorder (n = 672) | |
| Variant | P value | OR | P value | OR |
| rs148685782 | 0.135 | ↓0.920 | 0.072 | ↓0.509 |
| rs6063 | 0.063 | ↓0.907 | 0.032 | ↓0.454 |
| Gene Burden | 0.032 | ↓0.922 | 0.009 | ↓0.508 |

TABLE 2A
FGG neurological disease associations

| | | | | Alzheimer's | |
|---|---|---|---|---|---|
| | | | | Disease (n = 1,995) | |
| Variant | Gene | Function | AAF | P value | OR |
| rs148685782 | FGG | Missense A108G; | 0.004 | 0.031 | ↓0.562 |
| | | FGG IpQTL | | | |
| rs6063 | FGG | Missense G191R | 0.005 | 0.058 | ↓0.635 |
| Gene Burden | FGG | Aggregation of rare | 0.009 | 0.006 | ↓0.604 |
| | | deleterious variants | | | |

TABLE 2B
FGG neurological disease associations

| | Early-Onset | | | | | |
|---|---|---|---|---|---|---|
| | Alzheimer's | | | | | |
| | Disease | | Dementia | | Headache | |
| | (n = 190) | | (n = 4,283) | | (n = 25,534) | |
| Variant | P value | OR | P value | OR | P value | OR |
| rs148685782 | 0.275 | ↓0.513 | 0.034 | ↓0.739 | 3.03E−04 | ↓0.835 |
| rs6063 | 0.182 | ↓0.446 | 0.068 | ↓0.779 | 0.605 | ↓0.980 |
| Gene Burden | 0.026 | ↓0.229 | 0.012 | ↓0.783 | 0.004 | ↓0.911 |

The data demonstrated that there were protective associations with multiple psychiatric and depression-related traits (shown in Tables 1A-1C). The rs148685782 (A108G) variant, the rs6063 (G191R) variant, and the FGG gene burden were all associated with protection from major depressive disorder. Additionally, evaluated FGG variants were individually and collectively associated with decreased risk of SSRI medication-use, intentional self-harm, a family history of severe depression and post-traumatic stress disorder.

Additionally, there were protective associations with multiple neurological and dementia-related traits (shown in Table 2A-2B). The rs148685782 (A108G) variant, the rs6063 (G191R) variant and the FGG gene burden were individually and collectively associated with decreased risk of Alzheimer's Disease, early-onset Alzheimer's Disease, all-cause dementia and headache disorders.

These results indicate that loss-of-function of FGG resulted in protection from a range of psychiatric disorders, including depressive disorders, and from a range of neurological disorders, including Alzheimer's Disease; and suggest that therapeutic inhibition of FGG may result in similar disease-protective effects.

### Example 2: Bioinformatic Selection of Sequences in Order to Identify Therapeutic siRNAs to Downmodulate Expression of FGG mRNA

Screening sets were defined based on bioinformatic analysis. Therapeutic siRNAs were designed to target human FGG, and the FGG sequence of at least one toxicology-relevant species; in this case, non-human primates (NHP) including rhesus and cynomolgus monkeys. Drivers for the design of the screening set were predicted specificity of the siRNAs against the transcriptome of the relevant species as well as cross-reactivity between species. Predicted specificity in human, rhesus monkey, cynomolgus monkey, mouse, rat, rabbit, dog, gerbil, Syrian hamster, Chinese hamster, guinea pig, and naked mole rat was determined for sense (S) and antisense (AS) strands. These were assigned a “specificity score” which considered the likelihood of unintended downregulation of any other transcript by full or partial complementarity of an siRNA strand (up to 2 mismatches within positions 2-18) as well as the number and positions of mismatches. Thus, off-target(s) transcripts for antisense and sense strands of each siRNA were identified. In addition, the number of potential off-targets was used as an additional specificity factor in the specificity score. As identified, siRNAs with high specificity and a low number of predicted off-targets provide a benefit of increased targeting specificity.

In addition to selecting siRNA sequences with high sequence specificity to FGG mRNA, siRNA sequences within the seed region were analyzed for similarity to seed regions of known miRNAs. siRNAs can function in a miRNA like manner via base-pairing with complementary sequences within the 3′-UTR of mRNA molecules. The complementarity typically encompassed the 5′-bases at positions 2-7 of the miRNA (seed region). To circumvent siRNAs to act via functional miRNA binding sites, siRNA strands containing natural miRNA seed regions can be avoided. Seed regions identified in miRNAs from human, mouse, rat, rhesus monkey, dog, rabbit and pig are referred to as “conserved”. Combining the “specificity score” with miRNA seed analysis yielded a “specificity category”. This was divided into categories 1-4, with 1 having the highest specificity and 4 having the lowest specificity. Each strand of the siRNA was assigned to a specificity category.

Specificity and species cross-reactivity was assessed for human, rhesus monkey, cynomolgus monkey, mouse, rat, rabbit, dog, gerbil, Syrian hamster, Chinese hamster, guinea pig and naked mole rat FGG. The analysis was based on a canonical siRNA design using 19 bases and 17 bases (without considering positions 1 and 19) for cross-reactivity. Full match as well as single mismatch analyses were included.

Analysis of the Genome Aggregation Database (gnomAD, available at gnomad.broadinstitute.org/) to identify siRNAs targeting regions with known SNPs was also carried out to identify siRNAs that may be non-functional in individuals containing the SNP. Information regarding the positions of SNPs within the target sequence as well as minor allele frequency (MAF) in case data was obtained in this analysis.

Initial analysis of the relevant FGG mRNA sequence revealed few sequences that fulfil the specificity parameters and at the same time target FGG mRNA in all of the analyzed relevant species. Therefore, it was decided to design independent screening subsets for the therapeutic siRNAs.

The siRNAs in these subsets were selected based on the ability to recognize at least the human, cynomolgus monkey, rhesus monkey FGG sequences. Therefore, the siRNAs in these subsets may be used to target human FGG in a therapeutic setting.

The number of siRNA sequences derived from human FGG mRNA (ENST00000404648, SEQ ID NO: 3621) without consideration of specificity or species cross-reactivity was 1742 (sense and antisense strand sequences included in SEQ ID NOS: 1-3484).

Prioritizing sequences for target specificity, species cross-reactivity, miRNA seed region sequences and SNPs as described above yielded subset A. Subset A includes 319 siRNAs whose base sequences are shown in Table 3.

TABLE 3
Subset A siRNAs

| | Sense | | Antisense | |
|---|---|---|---|---|
| | strand | | strand | |
| siRNA | SEQ | Sense strand sequence | SEQ ID | Antisense strand sequence |
| Name | ID NO: | (5′-3′) | NO: | (5′-3′) |
| siRNA 224 | 224 | CCGGGCACTCAGACATCAT | 1966 | ATGATGTCTGAGTGCCCGG |
| siRNA 233 | 233 | CAGACATCATGAGTTGGTC | 1975 | GACCAACTCATGATGTCTG |
| siRNA 234 | 234 | AGACATCATGAGTTGGTCC | 1976 | GGACCAACTCATGATGTCT |
| siRNA 235 | 235 | GACATCATGAGTTGGTCCT | 1977 | AGGACCAACTCATGATGTC |
| siRNA 248 | 248 | GGTCCTTGCACCCCCGGAA | 1990 | TTCCGGGGGTGCAAGGACC |
| siRNA 249 | 249 | GTCCTTGCACCCCCGGAAT | 1991 | ATTCCGGGGGTGCAAGGAC |
| siRNA 250 | 250 | TCCTTGCACCCCCGGAATT | 1992 | AATTCCGGGGGTGCAAGGA |
| siRNA 251 | 251 | CCTTGCACCCCCGGAATTT | 1993 | AAATTCCGGGGGTGCAAGG |
| siRNA 253 | 253 | TTGCACCCCCGGAATTTAA | 1995 | TTAAATTCCGGGGGTGCAA |
| siRNA 254 | 254 | TGCACCCCCGGAATTTAAT | 1996 | ATTAAATTCCGGGGGTGCA |
| siRNA 255 | 255 | GCACCCCCGGAATTTAATT | 1997 | AATTAAATTCCGGGGGTGC |
| siRNA 258 | 258 | CCCCCGGAATTTAATTCTC | 2000 | GAGAATTAAATTCCGGGGG |
| siRNA 260 | 260 | CCCGGAATTTAATTCTCTA | 2002 | TAGAGAATTAAATTCCGGG |
| siRNA 261 | 261 | CCGGAATTTAATTCTCTAC | 2003 | GTAGAGAATTAAATTCCGG |
| siRNA 262 | 262 | CGGAATTTAATTCTCTACT | 2004 | AGTAGAGAATTAAATTCCG |
| siRNA 269 | 269 | TAATTCTCTACTTCTATGC | 2011 | GCATAGAAGTAGAGAATTA |
| siRNA 273 | 273 | TCTCTACTTCTATGCTCTT | 2015 | AAGAGCATAGAAGTAGAGA |
| siRNA 274 | 274 | CTCTACTTCTATGCTCTTT | 2016 | AAAGAGCATAGAAGTAGAG |
| siRNA 275 | 275 | TCTACTTCTATGCTCTTTT | 2017 | AAAAGAGCATAGAAGTAGA |
| siRNA 297 | 297 | TCTCTCTTCAACATGTGTA | 2039 | TACACATGTTGAAGAGAGA |
| siRNA 310 | 310 | TGTGTAGCATATGTTGCTA | 2052 | TAGCAACATATGCTACACA |
| siRNA 311 | 311 | GTGTAGCATATGTTGCTAC | 2053 | GTAGCAACATATGCTACAC |
| siRNA 319 | 319 | TATGTTGCTACCAGAGACA | 2061 | TGTCTCTGGTAGCAACATA |
| siRNA 322 | 322 | GTTGCTACCAGAGACAACT | 2064 | AGTTGTCTCTGGTAGCAAC |
| siRNA 323 | 323 | TTGCTACCAGAGACAACTG | 2065 | CAGTTGTCTCTGGTAGCAA |
| siRNA 324 | 324 | TGCTACCAGAGACAACTGC | 2066 | GCAGTTGTCTCTGGTAGCA |
| siRNA 329 | 329 | CCAGAGACAACTGCTGCAT | 2071 | ATGCAGCAGTTGTCTCTGG |
| siRNA 335 | 335 | ACAACTGCTGCATCTTAGA | 2077 | TCTAAGATGCAGCAGTTGT |
| siRNA 342 | 342 | CTGCATCTTAGATGAAAGA | 2084 | TCTTTCATCTAAGATGCAG |
| siRNA 343 | 343 | TGCATCTTAGATGAAAGAT | 2085 | ATCTTTCATCTAAGATGCA |
| siRNA 344 | 344 | GCATCTTAGATGAAAGATT | 2086 | AATCTTTCATCTAAGATGC |
| siRNA 347 | 347 | TCTTAGATGAAAGATTCGG | 2089 | CCGAATCTTTCATCTAAGA |
| siRNA 348 | 348 | CTTAGATGAAAGATTCGGT | 2090 | ACCGAATCTTTCATCTAAG |
| siRNA 349 | 349 | TTAGATGAAAGATTCGGTA | 2091 | TACCGAATCTTTCATCTAA |
| siRNA 350 | 350 | TAGATGAAAGATTCGGTAG | 2092 | CTACCGAATCTTTCATCTA |
| siRNA 351 | 351 | AGATGAAAGATTCGGTAGT | 2093 | ACTACCGAATCTTTCATCT |
| siRNA 352 | 352 | GATGAAAGATTCGGTAGTT | 2094 | AACTACCGAATCTTTCATC |
| siRNA 354 | 354 | TGAAAGATTCGGTAGTTAT | 2096 | ATAACTACCGAATCTTTCA |
| siRNA 355 | 355 | GAAAGATTCGGTAGTTATT | 2097 | AATAACTACCGAATCTTTC |
| siRNA 359 | 359 | GATTCGGTAGTTATTGTCC | 2101 | GGACAATAACTACCGAATC |
| siRNA 361 | 361 | TTCGGTAGTTATTGTCCAA | 2103 | TTGGACAATAACTACCGAA |
| siRNA 362 | 362 | TCGGTAGTTATTGTCCAAC | 2104 | GTTGGACAATAACTACCGA |
| siRNA 363 | 363 | CGGTAGTTATTGTCCAACT | 2105 | AGTTGGACAATAACTACCG |
| siRNA 364 | 364 | GGTAGTTATTGTCCAACTA | 2106 | TAGTTGGACAATAACTACC |
| siRNA 365 | 365 | GTAGTTATTGTCCAACTAC | 2107 | GTAGTTGGACAATAACTAC |
| siRNA 366 | 366 | TAGTTATTGTCCAACTACC | 2108 | GGTAGTTGGACAATAACTA |
| siRNA 367 | 367 | AGTTATTGTCCAACTACCT | 2109 | AGGTAGTTGGACAATAACT |
| siRNA 369 | 369 | TTATTGTCCAACTACCTGT | 2111 | ACAGGTAGTTGGACAATAA |
| siRNA 372 | 372 | TTGTCCAACTACCTGTGGC | 2114 | GCCACAGGTAGTTGGACAA |
| siRNA 373 | 373 | TGTCCAACTACCTGTGGCA | 2115 | TGCCACAGGTAGTTGGACA |
| siRNA 384 | 384 | CTGTGGCATTGCAGATTTC | 2126 | GAAATCTGCAATGCCACAG |
| siRNA 386 | 386 | GTGGCATTGCAGATTTCCT | 2128 | AGGAAATCTGCAATGCCAC |
| siRNA 392 | 392 | TTGCAGATTTCCTGTCTAC | 2134 | GTAGACAGGAAATCTGCAA |
| siRNA 397 | 397 | GATTTCCTGTCTACTTATC | 2139 | GATAAGTAGACAGGAAATC |
| siRNA 398 | 398 | ATTTCCTGTCTACTTATCA | 2140 | TGATAAGTAGACAGGAAAT |
| siRNA 399 | 399 | TTTCCTGTCTACTTATCAA | 2141 | TTGATAAGTAGACAGGAAA |
| siRNA 406 | 406 | TCTACTTATCAAACCAAAG | 2148 | CTTTGGTTTGATAAGTAGA |
| siRNA 411 | 411 | TTATCAAACCAAAGTAGAC | 2153 | GTCTACTTTGGTTTGATAA |
| siRNA 412 | 412 | TATCAAACCAAAGTAGACA | 2154 | TGTCTACTTTGGTTTGATA |
| siRNA 423 | 423 | AGTAGACAAGGATCTACAG | 2165 | CTGTAGATCCTTGTCTACT |
| siRNA 426 | 426 | AGACAAGGATCTACAGTCT | 2168 | AGACTGTAGATCCTTGTCT |
| siRNA 432 | 432 | GGATCTACAGTCTTTGGAA | 2174 | TTCCAAAGACTGTAGATCC |
| siRNA 434 | 434 | ATCTACAGTCTTTGGAAGA | 2176 | TCTTCCAAAGACTGTAGAT |
| siRNA 437 | 437 | TACAGTCTTTGGAAGACAT | 2179 | ATGTCTTCCAAAGACTGTA |
| siRNA 447 | 447 | GGAAGACATCTTACATCAA | 2189 | TTGATGTAAGATGTCTTCC |
| siRNA 449 | 449 | AAGACATCTTACATCAAGT | 2191 | ACTTGATGTAAGATGTCTT |
| siRNA 450 | 450 | AGACATCTTACATCAAGTT | 2192 | AACTTGATGTAAGATGTCT |
| siRNA 493 | 493 | CAGCTGATAAAAGCAATCC | 2235 | GGATTGCTTTTATCAGCTG |
| siRNA 494 | 494 | AGCTGATAAAAGCAATCCA | 2236 | TGGATTGCTTTTATCAGCT |
| siRNA 497 | 497 | TGATAAAAGCAATCCAACT | 2239 | AGTTGGATTGCTTTTATCA |
| siRNA 504 | 504 | AGCAATCCAACTCACTTAT | 2246 | ATAAGTGAGTTGGATTGCT |
| siRNA 505 | 505 | GCAATCCAACTCACTTATA | 2247 | TATAAGTGAGTTGGATTGC |
| siRNA 507 | 507 | AATCCAACTCACTTATAAT | 2249 | ATTATAAGTGAGTTGGATT |
| siRNA 508 | 508 | ATCCAACTCACTTATAATC | 2250 | GATTATAAGTGAGTTGGAT |
| siRNA 509 | 509 | TCCAACTCACTTATAATCC | 2251 | GGATTATAAGTGAGTTGGA |
| siRNA 510 | 510 | CCAACTCACTTATAATCCT | 2252 | AGGATTATAAGTGAGTTGG |
| siRNA 511 | 511 | CAACTCACTTATAATCCTG | 2253 | CAGGATTATAAGTGAGTTG |
| siRNA 514 | 514 | CTCACTTATAATCCTGATG | 2256 | CATCAGGATTATAAGTGAG |
| siRNA 515 | 515 | TCACTTATAATCCTGATGA | 2257 | TCATCAGGATTATAAGTGA |
| siRNA 522 | 522 | TAATCCTGATGAATCATCA | 2264 | TGATGATTCATCAGGATTA |
| siRNA 523 | 523 | AATCCTGATGAATCATCAA | 2265 | TTGATGATTCATCAGGATT |
| siRNA 528 | 528 | TGATGAATCATCAAAACCA | 2270 | TGGTTTTGATGATTCATCA |
| siRNA 539 | 539 | CAAAACCAAATATGATAGA | 2281 | TCTATCATATTTGGTTTTG |
| siRNA 541 | 541 | AAACCAAATATGATAGACG | 2283 | CGTCTATCATATTTGGTTT |
| siRNA 544 | 544 | CCAAATATGATAGACGCTG | 2286 | CAGCGTCTATCATATTTGG |
| siRNA 546 | 546 | AAATATGATAGACGCTGCT | 2288 | AGCAGCGTCTATCATATTT |
| siRNA 548 | 548 | ATATGATAGACGCTGCTAC | 2290 | GTAGCAGCGTCTATCATAT |
| siRNA 554 | 554 | TAGACGCTGCTACTTTGAA | 2296 | TTCAAAGTAGCAGCGTCTA |
| siRNA 556 | 556 | GACGCTGCTACTTTGAAGT | 2298 | ACTTCAAAGTAGCAGCGTC |
| siRNA 573 | 573 | GTCCAGGAAAATGTTAGAA | 2315 | TTCTAACATTTTCCTGGAC |
| siRNA 599 | 599 | TGAAATATGAAGCATCGAT | 2341 | ATCGATGCTTCATATTTCA |
| siRNA 600 | 600 | GAAATATGAAGCATCGATT | 2342 | AATCGATGCTTCATATTTC |
| siRNA 601 | 601 | AAATATGAAGCATCGATTT | 2343 | AAATCGATGCTTCATATTT |
| siRNA 604 | 604 | TATGAAGCATCGATTTTAA | 2346 | TTAAAATCGATGCTTCATA |
| siRNA 606 | 606 | TGAAGCATCGATTTTAACA | 2348 | TGTTAAAATCGATGCTTCA |
| siRNA 607 | 607 | GAAGCATCGATTTTAACAC | 2349 | GTGTTAAAATCGATGCTTC |
| siRNA 608 | 608 | AAGCATCGATTTTAACACA | 2350 | TGTGTTAAAATCGATGCTT |
| siRNA 609 | 609 | AGCATCGATTTTAACACAT | 2351 | ATGTGTTAAAATCGATGCT |
| siRNA 619 | 619 | TTAACACATGACTCAAGTA | 2361 | TACTTGAGTCATGTGTTAA |
| siRNA 624 | 624 | ACATGACTCAAGTATTCGA | 2366 | TCGAATACTTGAGTCATGT |
| siRNA 625 | 625 | CATGACTCAAGTATTCGAT | 2367 | ATCGAATACTTGAGTCATG |
| siRNA 633 | 633 | AAGTATTCGATATTTGCAG | 2375 | CTGCAAATATCGAATACTT |
| siRNA 634 | 634 | AGTATTCGATATTTGCAGG | 2376 | CCTGCAAATATCGAATACT |
| siRNA 661 | 661 | AATTCAAATAATCAAAAGA | 2403 | TCTTTTGATTATTTGAATT |
| siRNA 689 | 689 | TGAAAGAGAAGGTAGCCCA | 2431 | TGGGCTACCTTCTCTTTCA |
| siRNA 691 | 691 | AAAGAGAAGGTAGCCCAGC | 2433 | GCTGGGCTACCTTCTCTTT |
| siRNA 709 | 709 | CTTGAAGCACAGTGCCAGG | 2451 | CCTGGCACTGTGCTTCAAG |
| siRNA 716 | 716 | CACAGTGCCAGGAACCTTG | 2458 | CAAGGTTCCTGGCACTGTG |
| siRNA 720 | 720 | GTGCCAGGAACCTTGCAAA | 2462 | TTTGCAAGGTTCCTGGCAC |
| siRNA 724 | 724 | CAGGAACCTTGCAAAGACA | 2466 | TGTCTTTGCAAGGTTCCTG |
| siRNA 728 | 728 | AACCTTGCAAAGACACGGT | 2470 | ACCGTGTCTTTGCAAGGTT |
| siRNA 729 | 729 | ACCTTGCAAAGACACGGTG | 2471 | CACCGTGTCTTTGCAAGGT |
| siRNA 730 | 730 | CCTTGCAAAGACACGGTGC | 2472 | GCACCGTGTCTTTGCAAGG |
| siRNA 731 | 731 | CTTGCAAAGACACGGTGCA | 2473 | TGCACCGTGTCTTTGCAAG |
| siRNA 732 | 732 | TTGCAAAGACACGGTGCAA | 2474 | TTGCACCGTGTCTTTGCAA |
| siRNA 733 | 733 | TGCAAAGACACGGTGCAAA | 2475 | TTTGCACCGTGTCTTTGCA |
| siRNA 734 | 734 | GCAAAGACACGGTGCAAAT | 2476 | ATTTGCACCGTGTCTTTGC |
| siRNA 736 | 736 | AAAGACACGGTGCAAATCC | 2478 | GGATTTGCACCGTGTCTTT |
| siRNA 748 | 748 | CAAATCCATGATATCACTG | 2490 | CAGTGATATCATGGATTTG |
| siRNA 749 | 749 | AAATCCATGATATCACTGG | 2491 | CCAGTGATATCATGGATTT |
| siRNA 751 | 751 | ATCCATGATATCACTGGGA | 2493 | TCCCAGTGATATCATGGAT |
| siRNA 752 | 752 | TCCATGATATCACTGGGAA | 2494 | TTCCCAGTGATATCATGGA |
| siRNA 753 | 753 | CCATGATATCACTGGGAAA | 2495 | TTTCCCAGTGATATCATGG |
| siRNA 770 | 770 | AAGATTGTCAAGACATTGC | 2512 | GCAATGTCTTGACAATCTT |
| siRNA 778 | 778 | CAAGACATTGCCAATAAGG | 2520 | CCTTATTGGCAATGTCTTG |
| siRNA 780 | 780 | AGACATTGCCAATAAGGGA | 2522 | TCCCTTATTGGCAATGTCT |
| siRNA 783 | 783 | CATTGCCAATAAGGGAGCT | 2525 | AGCTCCCTTATTGGCAATG |
| siRNA 784 | 784 | ATTGCCAATAAGGGAGCTA | 2526 | TAGCTCCCTTATTGGCAAT |
| siRNA 785 | 785 | TTGCCAATAAGGGAGCTAA | 2527 | TTAGCTCCCTTATTGGCAA |
| siRNA 786 | 786 | TGCCAATAAGGGAGCTAAA | 2528 | TTTAGCTCCCTTATTGGCA |
| siRNA 791 | 791 | ATAAGGGAGCTAAACAGAG | 2533 | CTCTGTTTAGCTCCCTTAT |
| siRNA 794 | 794 | AGGGAGCTAAACAGAGCGG | 2536 | CCGCTCTGTTTAGCTCCCT |
| siRNA 795 | 795 | GGGAGCTAAACAGAGCGGG | 2537 | CCCGCTCTGTTTAGCTCCC |
| siRNA 796 | 796 | GGAGCTAAACAGAGCGGGC | 2538 | GCCCGCTCTGTTTAGCTCC |
| siRNA 797 | 797 | GAGCTAAACAGAGCGGGCT | 2539 | AGCCCGCTCTGTTTAGCTC |
| siRNA 800 | 800 | CTAAACAGAGCGGGCTTTA | 2542 | TAAAGCCCGCTCTGTTTAG |
| siRNA 802 | 802 | AAACAGAGCGGGCTTTACT | 2544 | AGTAAAGCCCGCTCTGTTT |
| siRNA 805 | 805 | CAGAGCGGGCTTTACTTTA | 2547 | TAAAGTAAAGCCCGCTCTG |
| siRNA 806 | 806 | AGAGCGGGCTTTACTTTAT | 2548 | ATAAAGTAAAGCCCGCTCT |
| siRNA 812 | 812 | GGCTTTACTTTATTAAACC | 2554 | GGTTTAATAAAGTAAAGCC |
| siRNA 821 | 821 | TTATTAAACCTCTGAAAGC | 2563 | GCTTTCAGAGGTTTAATAA |
| siRNA 822 | 822 | TATTAAACCTCTGAAAGCT | 2564 | AGCTTTCAGAGGTTTAATA |
| siRNA 825 | 825 | TAAACCTCTGAAAGCTAAC | 2567 | GTTAGCTTTCAGAGGTTTA |
| siRNA 826 | 826 | AAACCTCTGAAAGCTAACC | 2568 | GGTTAGCTTTCAGAGGTTT |
| siRNA 827 | 827 | AACCTCTGAAAGCTAACCA | 2569 | TGGTTAGCTTTCAGAGGTT |
| siRNA 828 | 828 | ACCTCTGAAAGCTAACCAG | 2570 | CTGGTTAGCTTTCAGAGGT |
| siRNA 833 | 833 | TGAAAGCTAACCAGCAATT | 2575 | AATTGCTGGTTAGCTTTCA |
| siRNA 841 | 841 | AACCAGCAATTCTTAGTCT | 2583 | AGACTAAGAATTGCTGGTT |
| siRNA 844 | 844 | CAGCAATTCTTAGTCTACT | 2586 | AGTAGACTAAGAATTGCTG |
| siRNA 850 | 850 | TTCTTAGTCTACTGTGAAA | 2592 | TTTCACAGTAGACTAAGAA |
| siRNA 860 | 860 | ACTGTGAAATCGATGGGTC | 2602 | GACCCATCGATTTCACAGT |
| siRNA 861 | 861 | CTGTGAAATCGATGGGTCT | 2603 | AGACCCATCGATTTCACAG |
| siRNA 865 | 865 | GAAATCGATGGGTCTGGAA | 2607 | TTCCAGACCCATCGATTTC |
| siRNA 866 | 866 | AAATCGATGGGTCTGGAAA | 2608 | TTTCCAGACCCATCGATTT |
| siRNA 877 | 877 | TCTGGAAATGGATGGACTG | 2619 | CAGTCCATCCATTTCCAGA |
| siRNA 893 | 893 | CTGTGTTTCAGAAGAGACT | 2635 | AGTCTCTTCTGAAACACAG |
| siRNA 894 | 894 | TGTGTTTCAGAAGAGACTT | 2636 | AAGTCTCTTCTGAAACACA |
| siRNA 904 | 904 | AAGAGACTTGATGGCAGTG | 2646 | CACTGCCATCAAGTCTCTT |
| siRNA 916 | 916 | GGCAGTGTAGATTTCAAGA | 2658 | TCTTGAAATCTACACTGCC |
| siRNA 929 | 929 | TCAAGAAAAACTGGATTCA | 2671 | TGAATCCAGTTTTTCTTGA |
| siRNA 932 | 932 | AGAAAAACTGGATTCAATA | 2674 | TATTGAATCCAGTTTTTCT |
| siRNA 955 | 955 | GAAGGATTTGGACATCTGT | 2697 | ACAGATGTCCAAATCCTTC |
| siRNA 956 | 956 | AAGGATTTGGACATCTGTC | 2698 | GACAGATGTCCAAATCCTT |
| siRNA 963 | 963 | TGGACATCTGTCTCCTACT | 2705 | AGTAGGAGACAGATGTCCA |
| siRNA 964 | 964 | GGACATCTGTCTCCTACTG | 2706 | CAGTAGGAGACAGATGTCC |
| siRNA 966 | 966 | ACATCTGTCTCCTACTGGC | 2708 | GCCAGTAGGAGACAGATGT |
| siRNA 969 | 969 | TCTGTCTCCTACTGGCACA | 2711 | TGTGCCAGTAGGAGACAGA |
| siRNA 972 | 972 | GTCTCCTACTGGCACAACA | 2714 | TGTTGTGCCAGTAGGAGAC |
| siRNA 973 | 973 | TCTCCTACTGGCACAACAG | 2715 | CTGTTGTGCCAGTAGGAGA |
| siRNA 983 | 983 | GCACAACAGAATTTTGGCT | 2725 | AGCCAAAATTCTGTTGTGC |
| siRNA 1003 | 1003 | GGAAATGAGAAGATTCATT | 2745 | AATGAATCTTCTCATTTCC |
| siRNA 1011 | 1011 | GAAGATTCATTTGATAAGC | 2753 | GCTTATCAAATGAATCTTC |
| siRNA 1013 | 1013 | AGATTCATTTGATAAGCAC | 2755 | GTGCTTATCAAATGAATCT |
| siRNA 1022 | 1022 | TGATAAGCACACAGTCTGC | 2764 | GCAGACTGTGTGCTTATCA |
| siRNA 1024 | 1024 | ATAAGCACACAGTCTGCCA | 2766 | TGGCAGACTGTGTGCTTAT |
| siRNA 1025 | 1025 | TAAGCACACAGTCTGCCAT | 2767 | ATGGCAGACTGTGTGCTTA |
| siRNA 1027 | 1027 | AGCACACAGTCTGCCATCC | 2769 | GGATGGCAGACTGTGTGCT |
| siRNA 1041 | 1041 | CATCCCATATGCATTAAGA | 2783 | TCTTAATGCATATGGGATG |
| siRNA 1046 | 1046 | CATATGCATTAAGAGTGGA | 2788 | TCCACTCTTAATGCATATG |
| siRNA 1047 | 1047 | ATATGCATTAAGAGTGGAA | 2789 | TTCCACTCTTAATGCATAT |
| siRNA 1052 | 1052 | CATTAAGAGTGGAACTGGA | 2794 | TCCAGTTCCACTCTTAATG |
| siRNA 1060 | 1060 | GTGGAACTGGAAGACTGGA | 2802 | TCCAGTCTTCCAGTTCCAC |
| siRNA 1071 | 1071 | AGACTGGAATGGCAGAACC | 2813 | GGTTCTGCCATTCCAGTCT |
| siRNA 1079 | 1079 | ATGGCAGAACCAGTACTGC | 2821 | GCAGTACTGGTTCTGCCAT |
| siRNA 1085 | 1085 | GAACCAGTACTGCAGACTA | 2827 | TAGTCTGCAGTACTGGTTC |
| siRNA 1088 | 1088 | CCAGTACTGCAGACTATGC | 2830 | GCATAGTCTGCAGTACTGG |
| siRNA 1089 | 1089 | CAGTACTGCAGACTATGCC | 2831 | GGCATAGTCTGCAGTACTG |
| siRNA 1091 | 1091 | GTACTGCAGACTATGCCAT | 2833 | ATGGCATAGTCTGCAGTAC |
| siRNA 1095 | 1095 | TGCAGACTATGCCATGTTC | 2837 | GAACATGGCATAGTCTGCA |
| siRNA 1110 | 1110 | GTTCAAGGTGGGACCTGAA | 2852 | TTCAGGTCCCACCTTGAAC |
| siRNA 1111 | 1111 | TTCAAGGTGGGACCTGAAG | 2853 | CTTCAGGTCCCACCTTGAA |
| siRNA 1120 | 1120 | GGACCTGAAGCTGACAAGT | 2862 | ACTTGTCAGCTTCAGGTCC |
| siRNA 1123 | 1123 | CCTGAAGCTGACAAGTACC | 2865 | GGTACTTGTCAGCTTCAGG |
| siRNA 1126 | 1126 | GAAGCTGACAAGTACCGCC | 2868 | GGCGGTACTTGTCAGCTTC |
| siRNA 1127 | 1127 | AAGCTGACAAGTACCGCCT | 2869 | AGGCGGTACTTGTCAGCTT |
| siRNA 1128 | 1128 | AGCTGACAAGTACCGCCTA | 2870 | TAGGCGGTACTTGTCAGCT |
| siRNA 1129 | 1129 | GCTGACAAGTACCGCCTAA | 2871 | TTAGGCGGTACTTGTCAGC |
| siRNA 1130 | 1130 | CTGACAAGTACCGCCTAAC | 2872 | GTTAGGCGGTACTTGTCAG |
| siRNA 1131 | 1131 | TGACAAGTACCGCCTAACA | 2873 | TGTTAGGCGGTACTTGTCA |
| siRNA 1132 | 1132 | GACAAGTACCGCCTAACAT | 2874 | ATGTTAGGCGGTACTTGTC |
| siRNA 1133 | 1133 | ACAAGTACCGCCTAACATA | 2875 | TATGTTAGGCGGTACTTGT |
| siRNA 1134 | 1134 | CAAGTACCGCCTAACATAT | 2876 | ATATGTTAGGCGGTACTTG |
| siRNA 1135 | 1135 | AAGTACCGCCTAACATATG | 2877 | CATATGTTAGGCGGTACTT |
| siRNA 1136 | 1136 | AGTACCGCCTAACATATGC | 2878 | GCATATGTTAGGCGGTACT |
| siRNA 1137 | 1137 | GTACCGCCTAACATATGCC | 2879 | GGCATATGTTAGGCGGTAC |
| siRNA 1138 | 1138 | TACCGCCTAACATATGCCT | 2880 | AGGCATATGTTAGGCGGTA |
| siRNA 1139 | 1139 | ACCGCCTAACATATGCCTA | 2881 | TAGGCATATGTTAGGCGGT |
| siRNA 1140 | 1140 | CCGCCTAACATATGCCTAC | 2882 | GTAGGCATATGTTAGGCGG |
| siRNA 1141 | 1141 | CGCCTAACATATGCCTACT | 2883 | AGTAGGCATATGTTAGGCG |
| siRNA 1143 | 1143 | CCTAACATATGCCTACTTC | 2885 | GAAGTAGGCATATGTTAGG |
| siRNA 1146 | 1146 | AACATATGCCTACTTCGCT | 2888 | AGCGAAGTAGGCATATGTT |
| siRNA 1147 | 1147 | ACATATGCCTACTTCGCTG | 2889 | CAGCGAAGTAGGCATATGT |
| siRNA 1148 | 1148 | CATATGCCTACTTCGCTGG | 2890 | CCAGCGAAGTAGGCATATG |
| siRNA 1149 | 1149 | ATATGCCTACTTCGCTGGT | 2891 | ACCAGCGAAGTAGGCATAT |
| siRNA 1151 | 1151 | ATGCCTACTTCGCTGGTGG | 2893 | CCACCAGCGAAGTAGGCAT |
| siRNA 1174 | 1174 | GCTGGAGATGCCTTTGATG | 2916 | CATCAAAGGCATCTCCAGC |
| siRNA 1178 | 1178 | GAGATGCCTTTGATGGCTT | 2920 | AAGCCATCAAAGGCATCTC |
| siRNA 1182 | 1182 | TGCCTTTGATGGCTTTGAT | 2924 | ATCAAAGCCATCAAAGGCA |
| siRNA 1197 | 1197 | TGATTTTGGCGATGATCCT | 2939 | AGGATCATCGCCAAAATCA |
| siRNA 1198 | 1198 | GATTTTGGCGATGATCCTA | 2940 | TAGGATCATCGCCAAAATC |
| siRNA 1203 | 1203 | TGGCGATGATCCTAGTGAC | 2945 | GTCACTAGGATCATCGCCA |
| siRNA 1205 | 1205 | GCGATGATCCTAGTGACAA | 2947 | TTGTCACTAGGATCATCGC |
| siRNA 1206 | 1206 | CGATGATCCTAGTGACAAG | 2948 | CTTGTCACTAGGATCATCG |
| siRNA 1215 | 1215 | TAGTGACAAGTTTTTCACA | 2957 | TGTGAAAAACTTGTCACTA |
| siRNA 1227 | 1227 | TTTCACATCCCATAATGGC | 2969 | GCCATTATGGGATGTGAAA |
| siRNA 1236 | 1236 | CCATAATGGCATGCAGTTC | 2978 | GAACTGCATGCCATTATGG |
| siRNA 1242 | 1242 | TGGCATGCAGTTCAGTACC | 2984 | GGTACTGAACTGCATGCCA |
| siRNA 1243 | 1243 | GGCATGCAGTTCAGTACCT | 2985 | AGGTACTGAACTGCATGCC |
| siRNA 1248 | 1248 | GCAGTTCAGTACCTGGGAC | 2990 | GTCCCAGGTACTGAACTGC |
| siRNA 1249 | 1249 | CAGTTCAGTACCTGGGACA | 2991 | TGTCCCAGGTACTGAACTG |
| siRNA 1264 | 1264 | GACAATGACAATGATAAGT | 3006 | ACTTATCATTGTCATTGTC |
| siRNA 1265 | 1265 | ACAATGACAATGATAAGTT | 3007 | AACTTATCATTGTCATTGT |
| siRNA 1278 | 1278 | TAAGTTTGAAGGCAACTGT | 3020 | ACAGTTGCCTTCAAACTTA |
| siRNA 1286 | 1286 | AAGGCAACTGTGCTGAACA | 3028 | TGTTCAGCACAGTTGCCTT |
| siRNA 1287 | 1287 | AGGCAACTGTGCTGAACAG | 3029 | CTGTTCAGCACAGTTGCCT |
| siRNA 1298 | 1298 | CTGAACAGGATGGATCTGG | 3040 | CCAGATCCATCCTGTTCAG |
| siRNA 1304 | 1304 | AGGATGGATCTGGTTGGTG | 3046 | CACCAACCAGATCCATCCT |
| siRNA 1311 | 1311 | ATCTGGTTGGTGGATGAAC | 3053 | GTTCATCCACCAACCAGAT |
| siRNA 1315 | 1315 | GGTTGGTGGATGAACAAGT | 3057 | ACTTGTTCATCCACCAACC |
| siRNA 1319 | 1319 | GGTGGATGAACAAGTGTCA | 3061 | TGACACTTGTTCATCCACC |
| siRNA 1320 | 1320 | GTGGATGAACAAGTGTCAC | 3062 | GTGACACTTGTTCATCCAC |
| siRNA 1323 | 1323 | GATGAACAAGTGTCACGCT | 3065 | AGCGTGACACTTGTTCATC |
| siRNA 1324 | 1324 | ATGAACAAGTGTCACGCTG | 3066 | CAGCGTGACACTTGTTCAT |
| siRNA 1325 | 1325 | TGAACAAGTGTCACGCTGG | 3067 | CCAGCGTGACACTTGTTCA |
| siRNA 1326 | 1326 | GAACAAGTGTCACGCTGGC | 3068 | GCCAGCGTGACACTTGTTC |
| siRNA 1339 | 1339 | GCTGGCCATCTCAATGGAG | 3081 | CTCCATTGAGATGGCCAGC |
| siRNA 1342 | 1342 | GGCCATCTCAATGGAGTTT | 3084 | AAACTCCATTGAGATGGCC |
| siRNA 1344 | 1344 | CCATCTCAATGGAGTTTAT | 3086 | ATAAACTCCATTGAGATGG |
| siRNA 1348 | 1348 | CTCAATGGAGTTTATTACC | 3090 | GGTAATAAACTCCATTGAG |
| siRNA 1349 | 1349 | TCAATGGAGTTTATTACCA | 3091 | TGGTAATAAACTCCATTGA |
| siRNA 1354 | 1354 | GGAGTTTATTACCAAGGTG | 3096 | CACCTTGGTAATAAACTCC |
| siRNA 1356 | 1356 | AGTTTATTACCAAGGTGGC | 3098 | GCCACCTTGGTAATAAACT |
| siRNA 1359 | 1359 | TTATTACCAAGGTGGCACT | 3101 | AGTGCCACCTTGGTAATAA |
| siRNA 1362 | 1362 | TTACCAAGGTGGCACTTAC | 3104 | GTAAGTGCCACCTTGGTAA |
| siRNA 1365 | 1365 | CCAAGGTGGCACTTACTCA | 3107 | TGAGTAAGTGCCACCTTGG |
| siRNA 1368 | 1368 | AGGTGGCACTTACTCAAAA | 3110 | TTTTGAGTAAGTGCCACCT |
| siRNA 1374 | 1374 | CACTTACTCAAAAGCATCT | 3116 | AGATGCTTTTGAGTAAGTG |
| siRNA 1383 | 1383 | AAAAGCATCTACTCCTAAT | 3125 | ATTAGGAGTAGATGCTTTT |
| siRNA 1384 | 1384 | AAAGCATCTACTCCTAATG | 3126 | CATTAGGAGTAGATGCTTT |
| siRNA 1385 | 1385 | AAGCATCTACTCCTAATGG | 3127 | CCATTAGGAGTAGATGCTT |
| siRNA 1386 | 1386 | AGCATCTACTCCTAATGGT | 3128 | ACCATTAGGAGTAGATGCT |
| siRNA 1387 | 1387 | GCATCTACTCCTAATGGTT | 3129 | AACCATTAGGAGTAGATGC |
| siRNA 1389 | 1389 | ATCTACTCCTAATGGTTAT | 3131 | ATAACCATTAGGAGTAGAT |
| siRNA 1390 | 1390 | TCTACTCCTAATGGTTATG | 3132 | CATAACCATTAGGAGTAGA |
| siRNA 1391 | 1391 | CTACTCCTAATGGTTATGA | 3133 | TCATAACCATTAGGAGTAG |
| siRNA 1394 | 1394 | CTCCTAATGGTTATGATAA | 3136 | TTATCATAACCATTAGGAG |
| siRNA 1395 | 1395 | TCCTAATGGTTATGATAAT | 3137 | ATTATCATAACCATTAGGA |
| siRNA 1396 | 1396 | CCTAATGGTTATGATAATG | 3138 | CATTATCATAACCATTAGG |
| siRNA 1401 | 1401 | TGGTTATGATAATGGCATT | 3143 | AATGCCATTATCATAACCA |
| siRNA 1404 | 1404 | TTATGATAATGGCATTATT | 3146 | AATAATGCCATTATCATAA |
| siRNA 1409 | 1409 | ATAATGGCATTATTTGGGC | 3151 | GCCCAAATAATGCCATTAT |
| siRNA 1410 | 1410 | TAATGGCATTATTTGGGCC | 3152 | GGCCCAAATAATGCCATTA |
| siRNA 1417 | 1417 | ATTATTTGGGCCACTTGGA | 3159 | TCCAAGTGGCCCAAATAAT |
| siRNA 1418 | 1418 | TTATTTGGGCCACTTGGAA | 3160 | TTCCAAGTGGCCCAAATAA |
| siRNA 1419 | 1419 | TATTTGGGCCACTTGGAAA | 3161 | TTTCCAAGTGGCCCAAATA |
| siRNA 1420 | 1420 | ATTTGGGCCACTTGGAAAA | 3162 | TTTTCCAAGTGGCCCAAAT |
| siRNA 1426 | 1426 | GCCACTTGGAAAACCCGGT | 3168 | ACCGGGTTTTCCAAGTGGC |
| siRNA 1427 | 1427 | CCACTTGGAAAACCCGGTG | 3169 | CACCGGGTTTTCCAAGTGG |
| siRNA 1428 | 1428 | CACTTGGAAAACCCGGTGG | 3170 | CCACCGGGTTTTCCAAGTG |
| siRNA 1431 | 1431 | TTGGAAAACCCGGTGGTAT | 3173 | ATACCACCGGGTTTTCCAA |
| siRNA 1432 | 1432 | TGGAAAACCCGGTGGTATT | 3174 | AATACCACCGGGTTTTCCA |
| siRNA 1434 | 1434 | GAAAACCCGGTGGTATTCC | 3176 | GGAATACCACCGGGTTTTC |
| siRNA 1435 | 1435 | AAAACCCGGTGGTATTCCA | 3177 | TGGAATACCACCGGGTTTT |
| siRNA 1440 | 1440 | CCGGTGGTATTCCATGAAG | 3182 | CTTCATGGAATACCACCGG |
| siRNA 1461 | 1461 | AACCACTATGAAGATAATC | 3203 | GATTATCTTCATAGTGGTT |
| siRNA 1462 | 1462 | ACCACTATGAAGATAATCC | 3204 | GGATTATCTTCATAGTGGT |
| siRNA 1466 | 1466 | CTATGAAGATAATCCCATT | 3208 | AATGGGATTATCTTCATAG |
| siRNA 1467 | 1467 | TATGAAGATAATCCCATTC | 3209 | GAATGGGATTATCTTCATA |
| siRNA 1477 | 1477 | ATCCCATTCAACAGACTCA | 3219 | TGAGTCTGTTGAATGGGAT |
| siRNA 1478 | 1478 | TCCCATTCAACAGACTCAC | 3220 | GTGAGTCTGTTGAATGGGA |
| siRNA 1482 | 1482 | ATTCAACAGACTCACAATT | 3224 | AATTGTGAGTCTGTTGAAT |
| siRNA 1485 | 1485 | CAACAGACTCACAATTGGA | 3227 | TCCAATTGTGAGTCTGTTG |
| siRNA 1487 | 1487 | ACAGACTCACAATTGGAGA | 3229 | TCTCCAATTGTGAGTCTGT |
| siRNA 1488 | 1488 | CAGACTCACAATTGGAGAA | 3230 | TTCTCCAATTGTGAGTCTG |
| siRNA 1519 | 1519 | CACCTGGGGGGAGCCAAAC | 3261 | GTTTGGCTCCCCCCAGGTG |
| siRNA 1533 | 1533 | CAAACAGGCTGGAGACGTT | 3275 | AACGTCTCCAGCCTGTTTG |
| siRNA 1540 | 1540 | GCTGGAGACGTTTAAAAGA | 3282 | TCTTTTAAACGTCTCCAGC |
| siRNA 1583 | 1583 | TTAAAGGACTTTATCTGAA | 3325 | TTCAGATAAAGTCCTTTAA |
| siRNA 1588 | 1588 | GGACTTTATCTGAACAGAG | 3330 | CTCTGTTCAGATAAAGTCC |
| siRNA 1592 | 1592 | TTTATCTGAACAGAGAGAT | 3334 | ATCTCTCTGTTCAGATAAA |
| siRNA 1597 | 1597 | CTGAACAGAGAGATATAAT | 3339 | ATTATATCTCTCTGTTCAG |
| siRNA 1615 | 1615 | TATTTTTCCTATTGGACAA | 3357 | TTGTCCAATAGGAAAAATA |
| siRNA 1617 | 1617 | TTTTTCCTATTGGACAATG | 3359 | CATTGTCCAATAGGAAAAA |
| siRNA 1622 | 1622 | CCTATTGGACAATGGACTT | 3364 | AAGTCCATTGTCCAATAGG |
| siRNA 1623 | 1623 | CTATTGGACAATGGACTTG | 3365 | CAAGTCCATTGTCCAATAG |
| siRNA 1626 | 1626 | TTGGACAATGGACTTGCAA | 3368 | TTGCAAGTCCATTGTCCAA |
| siRNA 1627 | 1627 | TGGACAATGGACTTGCAAA | 3369 | TTTGCAAGTCCATTGTCCA |
| siRNA 1647 | 1647 | CTTCACTTCATTTTAAGAG | 3389 | CTCTTAAAATGAAGTGAAG |
| siRNA 1648 | 1648 | TTCACTTCATTTTAAGAGC | 3390 | GCTCTTAAAATGAAGTGAA |
| siRNA 1649 | 1649 | TCACTTCATTTTAAGAGCA | 3391 | TGCTCTTAAAATGAAGTGA |
| siRNA 1664 | 1664 | AGCAAAAGACCCCATGTTG | 3406 | CAACATGGGGTCTTTTGCT |
| siRNA 1672 | 1672 | ACCCCATGTTGAAAACTCC | 3414 | GGAGTTTTCAACATGGGGT |
| siRNA 1678 | 1678 | TGTTGAAAACTCCATAACA | 3420 | TGTTATGGAGTTTTCAACA |
| siRNA 1679 | 1679 | GTTGAAAACTCCATAACAG | 3421 | CTGTTATGGAGTTTTCAAC |
| siRNA 1680 | 1680 | TTGAAAACTCCATAACAGT | 3422 | ACTGTTATGGAGTTTTCAA |
| siRNA 1691 | 1691 | ATAACAGTTTTATGCTGAT | 3433 | ATCAGCATAAAACTGTTAT |
| siRNA 1696 | 1696 | AGTTTTATGCTGATGATAA | 3438 | TTATCATCAGCATAAAACT |
| siRNA 1700 | 1700 | TTATGCTGATGATAATTTA | 3442 | TAAATTATCATCAGCATAA |
| siRNA 1710 | 1710 | GATAATTTATCTACATGCA | 3452 | TGCATGTAGATAAATTATC |

The siRNAs in subset A were selected to have the following characteristics:
Cross-reactivity: With 19mer in human FGG mRNA, with 17mer/19mer in NHP FGG
Specificity category: For human and NHP: AS2 or better, SS3 or better
miRNA seeds: AS+SS strand: seed region not conserved in human, mouse, and rat and not present in >4 species
Off-target frequency: ≤30 human off-targets matched with 2 mismatches in antisense strand
SNPs: siRNA target sites do not harbor SNPs with a MAF≥1% (pos. 2-18)

The siRNA sequences in subset A were selected for more stringent specificity to yield subset B. Subset B includes 318 siRNAs whose base sequences are shown in Table 4.

TABLE 4
Subset B siRNAs

| | Sense | | Antisense | |
|---|---|---|---|---|
| | strand | | strand | |
| siRNA | SEQ ID | Sense strand sequence | SEQ ID | Antisense strand sequence |
| Name | NO: | (5′-3′) | NO: | (5′-3′) |
| siRNA 224 | 224 | CCGGGCACTCAGACATCAT | 1966 | ATGATGTCTGAGTGCCCGG |
| siRNA 233 | 233 | CAGACATCATGAGTTGGTC | 1975 | GACCAACTCATGATGTCTG |
| siRNA 234 | 234 | AGACATCATGAGTTGGTCC | 1976 | GGACCAACTCATGATGTCT |
| siRNA 235 | 235 | GACATCATGAGTTGGTCCT | 1977 | AGGACCAACTCATGATGTC |
| siRNA 248 | 248 | GGTCCTTGCACCCCCGGAA | 1990 | TTCCGGGGGTGCAAGGACC |
| siRNA 249 | 249 | GTCCTTGCACCCCCGGAAT | 1991 | ATTCCGGGGGTGCAAGGAC |
| siRNA 250 | 250 | TCCTTGCACCCCCGGAATT | 1992 | AATTCCGGGGGTGCAAGGA |
| siRNA 251 | 251 | CCTTGCACCCCCGGAATTT | 1993 | AAATTCCGGGGGTGCAAGG |
| siRNA 253 | 253 | TTGCACCCCCGGAATTTAA | 1995 | TTAAATTCCGGGGGTGCAA |
| siRNA 254 | 254 | TGCACCCCCGGAATTTAAT | 1996 | ATTAAATTCCGGGGGTGCA |
| siRNA 255 | 255 | GCACCCCCGGAATTTAATT | 1997 | AATTAAATTCCGGGGGTGC |
| siRNA 258 | 258 | CCCCCGGAATTTAATTCTC | 2000 | GAGAATTAAATTCCGGGGG |
| siRNA 260 | 260 | CCCGGAATTTAATTCTCTA | 2002 | TAGAGAATTAAATTCCGGG |
| siRNA 261 | 261 | CCGGAATTTAATTCTCTAC | 2003 | GTAGAGAATTAAATTCCGG |
| siRNA 262 | 262 | CGGAATTTAATTCTCTACT | 2004 | AGTAGAGAATTAAATTCCG |
| siRNA 269 | 269 | TAATTCTCTACTTCTATGC | 2011 | GCATAGAAGTAGAGAATTA |
| siRNA 273 | 273 | TCTCTACTTCTATGCTCTT | 2015 | AAGAGCATAGAAGTAGAGA |
| siRNA 274 | 274 | CTCTACTTCTATGCTCTTT | 2016 | AAAGAGCATAGAAGTAGAG |
| siRNA 275 | 275 | TCTACTTCTATGCTCTTTT | 2017 | AAAAGAGCATAGAAGTAGA |
| siRNA 297 | 297 | TCTCTCTTCAACATGTGTA | 2039 | TACACATGTTGAAGAGAGA |
| siRNA 310 | 310 | TGTGTAGCATATGTTGCTA | 2052 | TAGCAACATATGCTACACA |
| siRNA 311 | 311 | GTGTAGCATATGTTGCTAC | 2053 | GTAGCAACATATGCTACAC |
| siRNA 319 | 319 | TATGTTGCTACCAGAGACA | 2061 | TGTCTCTGGTAGCAACATA |
| siRNA 322 | 322 | GTTGCTACCAGAGACAACT | 2064 | AGTTGTCTCTGGTAGCAAC |
| siRNA 323 | 323 | TTGCTACCAGAGACAACTG | 2065 | CAGTTGTCTCTGGTAGCAA |
| siRNA 324 | 324 | TGCTACCAGAGACAACTGC | 2066 | GCAGTIGTCTCTGGTAGCA |
| siRNA 329 | 329 | CCAGAGACAACTGCTGCAT | 2071 | ATGCAGCAGTTGTCTCTGG |
| siRNA 335 | 335 | ACAACTGCTGCATCTTAGA | 2077 | TCTAAGATGCAGCAGTTGT |
| siRNA 342 | 342 | CTGCATCTTAGATGAAAGA | 2084 | TCTTTCATCTAAGATGCAG |
| siRNA 343 | 343 | TGCATCTTAGATGAAAGAT | 2085 | ATCTTTCATCTAAGATGCA |
| siRNA 344 | 344 | GCATCTTAGATGAAAGATT | 2086 | AATCTTTCATCTAAGATGC |
| siRNA 347 | 347 | TCTTAGATGAAAGATTCGG | 2089 | CCGAATCTTTCATCTAAGA |
| siRNA 348 | 348 | CTTAGATGAAAGATTCGGT | 2090 | ACCGAATCTTTCATCTAAG |
| siRNA 349 | 349 | TTAGATGAAAGATTCGGTA | 2091 | TACCGAATCTTTCATCTAA |
| siRNA 350 | 350 | TAGATGAAAGATTCGGTAG | 2092 | CTACCGAATCTTTCATCTA |
| siRNA 351 | 351 | AGATGAAAGATTCGGTAGT | 2093 | ACTACCGAATCTTTCATCT |
| siRNA 352 | 352 | GATGAAAGATTCGGTAGTT | 2094 | AACTACCGAATCTTTCATC |
| siRNA 354 | 354 | TGAAAGATTCGGTAGTTAT | 2096 | ATAACTACCGAATCTTTCA |
| siRNA 355 | 355 | GAAAGATTCGGTAGTTATT | 2097 | AATAACTACCGAATCTTTC |
| siRNA 359 | 359 | GATTCGGTAGTTATTGTCC | 2101 | GGACAATAACTACCGAATC |
| siRNA 361 | 361 | TTCGGTAGTTATTGTCCAA | 2103 | TTGGACAATAACTACCGAA |
| siRNA 362 | 362 | TCGGTAGTTATTGTCCAAC | 2104 | GTTGGACAATAACTACCGA |
| siRNA 363 | 363 | CGGTAGTTATTGTCCAACT | 2105 | AGTTGGACAATAACTACCG |
| siRNA 364 | 364 | GGTAGTTATTGTCCAACTA | 2106 | TAGTTGGACAATAACTACC |
| siRNA 365 | 365 | GTAGTTATTGTCCAACTAC | 2107 | GTAGTTGGACAATAACTAC |
| siRNA 366 | 366 | TAGTTATTGTCCAACTACC | 2108 | GGTAGTTGGACAATAACTA |
| siRNA 367 | 367 | AGTTATTGTCCAACTACCT | 2109 | AGGTAGTTGGACAATAACT |
| siRNA 369 | 369 | TTATTGTCCAACTACCTGT | 2111 | ACAGGTAGTTGGACAATAA |
| siRNA 372 | 372 | TTGTCCAACTACCTGTGGC | 2114 | GCCACAGGTAGTTGGACAA |
| siRNA 373 | 373 | TGTCCAACTACCTGTGGCA | 2115 | TGCCACAGGTAGTTGGACA |
| siRNA 384 | 384 | CTGTGGCATTGCAGATTTC | 2126 | GAAATCTGCAATGCCACAG |
| siRNA 386 | 386 | GTGGCATTGCAGATTTCCT | 2128 | AGGAAATCTGCAATGCCAC |
| siRNA 392 | 392 | TTGCAGATTTCCTGTCTAC | 2134 | GTAGACAGGAAATCTGCAA |
| siRNA 397 | 397 | GATTTCCTGTCTACTTATC | 2139 | GATAAGTAGACAGGAAATC |
| siRNA 398 | 398 | ATTTCCTGTCTACTTATCA | 2140 | TGATAAGTAGACAGGAAAT |
| siRNA 399 | 399 | TTTCCTGTCTACTTATCAA | 2141 | TTGATAAGTAGACAGGAAA |
| siRNA 406 | 406 | TCTACTTATCAAACCAAAG | 2148 | CTTTGGTTTGATAAGTAGA |
| siRNA 411 | 411 | TTATCAAACCAAAGTAGAC | 2153 | GTCTACTTTGGTTTGATAA |
| siRNA 412 | 412 | TATCAAACCAAAGTAGACA | 2154 | TGTCTACTTTGGTTTGATA |
| siRNA 423 | 423 | AGTAGACAAGGATCTACAG | 2165 | CTGTAGATCCTTGTCTACT |
| siRNA 426 | 426 | AGACAAGGATCTACAGTCT | 2168 | AGACTGTAGATCCTTGTCT |
| siRNA 432 | 432 | GGATCTACAGTCTTTGGAA | 2174 | TTCCAAAGACTGTAGATCC |
| siRNA 434 | 434 | ATCTACAGTCTTTGGAAGA | 2176 | TCTTCCAAAGACTGTAGAT |
| siRNA 437 | 437 | TACAGTCTTTGGAAGACAT | 2179 | ATGTCTTCCAAAGACTGTA |
| siRNA 447 | 447 | GGAAGACATCTTACATCAA | 2189 | TTGATGTAAGATGTCTTCC |
| siRNA 449 | 449 | AAGACATCTTACATCAAGT | 2191 | ACTTGATGTAAGATGTCTT |
| siRNA 450 | 450 | AGACATCTTACATCAAGTT | 2192 | AACTTGATGTAAGATGTCT |
| siRNA 493 | 493 | CAGCTGATAAAAGCAATCC | 2235 | GGATTGCTTTTATCAGCTG |
| siRNA 494 | 494 | AGCTGATAAAAGCAATCCA | 2236 | TGGATTGCTTTTATCAGCT |
| siRNA 497 | 497 | TGATAAAAGCAATCCAACT | 2239 | AGTTGGATTGCTTTTATCA |
| siRNA 504 | 504 | AGCAATCCAACTCACTTAT | 2246 | ATAAGTGAGTTGGATTGCT |
| siRNA 505 | 505 | GCAATCCAACTCACTTATA | 2247 | TATAAGTGAGTTGGATTGC |
| siRNA 507 | 507 | AATCCAACTCACTTATAAT | 2249 | ATTATAAGTGAGTTGGATT |
| siRNA 508 | 508 | ATCCAACTCACTTATAATC | 2250 | GATTATAAGTGAGTTGGAT |
| siRNA 509 | 509 | TCCAACTCACTTATAATCC | 2251 | GGATTATAAGTGAGTTGGA |
| siRNA 510 | 510 | CCAACTCACTTATAATCCT | 2252 | AGGATTATAAGTGAGTTGG |
| siRNA 511 | 511 | CAACTCACTTATAATCCTG | 2253 | CAGGATTATAAGTGAGTTG |
| siRNA 514 | 514 | CTCACTTATAATCCTGATG | 2256 | CATCAGGATTATAAGTGAG |
| siRNA 515 | 515 | TCACTTATAATCCTGATGA | 2257 | TCATCAGGATTATAAGTGA |
| siRNA 522 | 522 | TAATCCTGATGAATCATCA | 2264 | TGATGATTCATCAGGATTA |
| siRNA 523 | 523 | AATCCTGATGAATCATCAA | 2265 | TTGATGATTCATCAGGATT |
| siRNA 528 | 528 | TGATGAATCATCAAAACCA | 2270 | TGGTTTTGATGATTCATCA |
| siRNA 539 | 539 | CAAAACCAAATATGATAGA | 2281 | TCTATCATATTTGGTTTTG |
| siRNA 541 | 541 | AAACCAAATATGATAGACG | 2283 | CGTCTATCATATTTGGTTT |
| siRNA 544 | 544 | CCAAATATGATAGACGCTG | 2286 | CAGCGTCTATCATATTTGG |
| siRNA 546 | 546 | AAATATGATAGACGCTGCT | 2288 | AGCAGCGTCTATCATATTT |
| siRNA 548 | 548 | ATATGATAGACGCTGCTAC | 2290 | GTAGCAGCGTCTATCATAT |
| siRNA 554 | 554 | TAGACGCTGCTACTTTGAA | 2296 | TTCAAAGTAGCAGCGTCTA |
| siRNA 556 | 556 | GACGCTGCTACTTTGAAGT | 2298 | ACTTCAAAGTAGCAGCGTC |
| siRNA 573 | 573 | GTCCAGGAAAATGTTAGAA | 2315 | TTCTAACATTTTCCTGGAC |
| siRNA 599 | 599 | TGAAATATGAAGCATCGAT | 2341 | ATCGATGCTTCATATTTCA |
| siRNA 600 | 600 | GAAATATGAAGCATCGATT | 2342 | AATCGATGCTTCATATTTC |
| siRNA 601 | 601 | AAATATGAAGCATCGATTT | 2343 | AAATCGATGCTTCATATTT |
| siRNA 604 | 604 | TATGAAGCATCGATTTTAA | 2346 | TTAAAATCGATGCTTCATA |
| siRNA 606 | 606 | TGAAGCATCGATTTTAACA | 2348 | TGTTAAAATCGATGCTTCA |
| siRNA 607 | 607 | GAAGCATCGATTTTAACAC | 2349 | GTGTTAAAATCGATGCTTC |
| siRNA 608 | 608 | AAGCATCGATTTTAACACA | 2350 | TGTGTTAAAATCGATGCTT |
| siRNA 609 | 609 | AGCATCGATTTTAACACAT | 2351 | ATGTGTTAAAATCGATGCT |
| siRNA 619 | 619 | TTAACACATGACTCAAGTA | 2361 | TACTTGAGTCATGTGTTAA |
| siRNA 624 | 624 | ACATGACTCAAGTATTCGA | 2366 | TCGAATACTTGAGTCATGT |
| siRNA 625 | 625 | CATGACTCAAGTATTCGAT | 2367 | ATCGAATACTTGAGTCATG |
| siRNA 633 | 633 | AAGTATTCGATATTTGCAG | 2375 | CTGCAAATATCGAATACTT |
| siRNA 634 | 634 | AGTATTCGATATTTGCAGG | 2376 | CCTGCAAATATCGAATACT |
| siRNA 689 | 689 | TGAAAGAGAAGGTAGCCCA | 2431 | TGGGCTACCTTCTCTTTCA |
| siRNA 691 | 691 | AAAGAGAAGGTAGCCCAGC | 2433 | GCTGGGCTACCTTCTCTTT |
| siRNA 709 | 709 | CTTGAAGCACAGTGCCAGG | 2451 | CCTGGCACTGTGCTTCAAG |
| siRNA 716 | 716 | CACAGTGCCAGGAACCTTG | 2458 | CAAGGTTCCTGGCACTGTG |
| siRNA 720 | 720 | GTGCCAGGAACCTTGCAAA | 2462 | TTTGCAAGGTTCCTGGCAC |
| siRNA 724 | 724 | CAGGAACCTTGCAAAGACA | 2466 | TGTCTTTGCAAGGTTCCTG |
| siRNA 728 | 728 | AACCTTGCAAAGACACGGT | 2470 | ACCGTGTCTTTGCAAGGTT |
| siRNA 729 | 729 | ACCTTGCAAAGACACGGTG | 2471 | CACCGTGTCTTTGCAAGGT |
| siRNA 730 | 730 | CCTTGCAAAGACACGGTGC | 2472 | GCACCGTGTCTTTGCAAGG |
| siRNA 731 | 731 | CTTGCAAAGACACGGTGCA | 2473 | TGCACCGTGTCTTTGCAAG |
| siRNA 732 | 732 | TTGCAAAGACACGGTGCAA | 2474 | TTGCACCGTGTCTTTGCAA |
| siRNA 733 | 733 | TGCAAAGACACGGTGCAAA | 2475 | TTTGCACCGTGTCTTTGCA |
| siRNA 734 | 734 | GCAAAGACACGGTGCAAAT | 2476 | ATTTGCACCGTGTCTTTGC |
| siRNA 736 | 736 | AAAGACACGGTGCAAATCC | 2478 | GGATTTGCACCGTGTCTTT |
| siRNA 748 | 748 | CAAATCCATGATATCACTG | 2490 | CAGTGATATCATGGATTTG |
| siRNA 749 | 749 | AAATCCATGATATCACTGG | 2491 | CCAGTGATATCATGGATTT |
| siRNA 751 | 751 | ATCCATGATATCACTGGGA | 2493 | TCCCAGTGATATCATGGAT |
| siRNA 752 | 752 | TCCATGATATCACTGGGAA | 2494 | TTCCCAGTGATATCATGGA |
| siRNA 753 | 753 | CCATGATATCACTGGGAAA | 2495 | TTTCCCAGTGATATCATGG |
| siRNA 770 | 770 | AAGATTGTCAAGACATTGC | 2512 | GCAATGTCTTGACAATCTT |
| siRNA 778 | 778 | CAAGACATTGCCAATAAGG | 2520 | CCTTATTGGCAATGTCTTG |
| siRNA 780 | 780 | AGACATTGCCAATAAGGGA | 2522 | TCCCTTATTGGCAATGTCT |
| siRNA 783 | 783 | CATTGCCAATAAGGGAGCT | 2525 | AGCTCCCTTATTGGCAATG |
| siRNA 784 | 784 | ATTGCCAATAAGGGAGCTA | 2526 | TAGCTCCCTTATTGGCAAT |
| siRNA 785 | 785 | TTGCCAATAAGGGAGCTAA | 2527 | TTAGCTCCCTTATTGGCAA |
| siRNA 786 | 786 | TGCCAATAAGGGAGCTAAA | 2528 | TTTAGCTCCCTTATTGGCA |
| siRNA 791 | 791 | ATAAGGGAGCTAAACAGAG | 2533 | CTCTGTTTAGCTCCCTTAT |
| siRNA 794 | 794 | AGGGAGCTAAACAGAGCGG | 2536 | CCGCTCTGTTTAGCTCCCT |
| siRNA 795 | 795 | GGGAGCTAAACAGAGCGGG | 2537 | CCCGCTCTGTTTAGCTCCC |
| siRNA 796 | 796 | GGAGCTAAACAGAGCGGGC | 2538 | GCCCGCTCTGTTTAGCTCC |
| siRNA 797 | 797 | GAGCTAAACAGAGCGGGCT | 2539 | AGCCCGCTCTGTTTAGCTC |
| siRNA 800 | 800 | CTAAACAGAGCGGGCTTTA | 2542 | TAAAGCCCGCTCTGTTTAG |
| siRNA 802 | 802 | AAACAGAGCGGGCTTTACT | 2544 | AGTAAAGCCCGCTCTGTTT |
| siRNA 805 | 805 | CAGAGCGGGCTTTACTTTA | 2547 | TAAAGTAAAGCCCGCTCTG |
| siRNA 806 | 806 | AGAGCGGGCTTTACTTTAT | 2548 | ATAAAGTAAAGCCCGCTCT |
| siRNA 812 | 812 | GGCTTTACTTTATTAAACC | 2554 | GGTTTAATAAAGTAAAGCC |
| siRNA 821 | 821 | TTATTAAACCTCTGAAAGC | 2563 | GCTTTCAGAGGTTTAATAA |
| siRNA 822 | 822 | TATTAAACCTCTGAAAGCT | 2564 | AGCTTTCAGAGGTTTAATA |
| siRNA 825 | 825 | TAAACCTCTGAAAGCTAAC | 2567 | GTTAGCTTTCAGAGGTTTA |
| siRNA 826 | 826 | AAACCTCTGAAAGCTAACC | 2568 | GGTTAGCTTTCAGAGGTTT |
| siRNA 827 | 827 | AACCTCTGAAAGCTAACCA | 2569 | TGGTTAGCTTTCAGAGGTT |
| siRNA 828 | 828 | ACCTCTGAAAGCTAACCAG | 2570 | CTGGTTAGCTTTCAGAGGT |
| siRNA 833 | 833 | TGAAAGCTAACCAGCAATT | 2575 | AATTGCTGGTTAGCTTTCA |
| siRNA 841 | 841 | AACCAGCAATTCTTAGTCT | 2583 | AGACTAAGAATTGCTGGTT |
| siRNA 844 | 844 | CAGCAATTCTTAGTCTACT | 2586 | AGTAGACTAAGAATTGCTG |
| siRNA 850 | 850 | TTCTTAGTCTACTGTGAAA | 2592 | TTTCACAGTAGACTAAGAA |
| siRNA 860 | 860 | ACTGTGAAATCGATGGGTC | 2602 | GACCCATCGATTTCACAGT |
| siRNA 861 | 861 | CTGTGAAATCGATGGGTCT | 2603 | AGACCCATCGATTTCACAG |
| siRNA 865 | 865 | GAAATCGATGGGTCTGGAA | 2607 | TTCCAGACCCATCGATTTC |
| siRNA 866 | 866 | AAATCGATGGGTCTGGAAA | 2608 | TTTCCAGACCCATCGATTT |
| siRNA 877 | 877 | TCTGGAAATGGATGGACTG | 2619 | CAGTCCATCCATTTCCAGA |
| siRNA 893 | 893 | CTGTGTTTCAGAAGAGACT | 2635 | AGTCTCTTCTGAAACACAG |
| siRNA 894 | 894 | TGTGTTTCAGAAGAGACTT | 2636 | AAGTCTCTTCTGAAACACA |
| siRNA 904 | 904 | AAGAGACTTGATGGCAGTG | 2646 | CACTGCCATCAAGTCTCTT |
| siRNA 916 | 916 | GGCAGTGTAGATTTCAAGA | 2658 | TCTTGAAATCTACACTGCC |
| siRNA 929 | 929 | TCAAGAAAAACTGGATTCA | 2671 | TGAATCCAGTTTTTCTTGA |
| siRNA 932 | 932 | AGAAAAACTGGATTCAATA | 2674 | TATTGAATCCAGTTTTTCT |
| siRNA 955 | 955 | GAAGGATTTGGACATCTGT | 2697 | ACAGATGTCCAAATCCTTC |
| siRNA 956 | 956 | AAGGATTTGGACATCTGTC | 2698 | GACAGATGTCCAAATCCTT |
| siRNA 963 | 963 | TGGACATCTGTCTCCTACT | 2705 | AGTAGGAGACAGATGTCCA |
| siRNA 964 | 964 | GGACATCTGTCTCCTACTG | 2706 | CAGTAGGAGACAGATGTCC |
| siRNA 966 | 966 | ACATCTGTCTCCTACTGGC | 2708 | GCCAGTAGGAGACAGATGT |
| siRNA 969 | 969 | TCTGTCTCCTACTGGCACA | 2711 | TGTGCCAGTAGGAGACAGA |
| siRNA 972 | 972 | GTCTCCTACTGGCACAACA | 2714 | TGTTGTGCCAGTAGGAGAC |
| siRNA 973 | 973 | TCTCCTACTGGCACAACAG | 2715 | CTGTTGTGCCAGTAGGAGA |
| siRNA 983 | 983 | GCACAACAGAATTTTGGCT | 2725 | AGCCAAAATTCTGTIGTGC |
| siRNA 1003 | 1003 | GGAAATGAGAAGATTCATT | 2745 | AATGAATCTTCTCATTTCC |
| siRNA 1011 | 1011 | GAAGATTCATTTGATAAGC | 2753 | GCTTATCAAATGAATCTTC |
| siRNA 1013 | 1013 | AGATTCATTTGATAAGCAC | 2755 | GTGCTTATCAAATGAATCT |
| siRNA 1022 | 1022 | TGATAAGCACACAGTCTGC | 2764 | GCAGACTGTGTGCTTATCA |
| siRNA 1024 | 1024 | ATAAGCACACAGTCTGCCA | 2766 | TGGCAGACTGTGTGCTTAT |
| siRNA 1025 | 1025 | TAAGCACACAGTCTGCCAT | 2767 | ATGGCAGACTGTGTGCTTA |
| siRNA 1027 | 1027 | AGCACACAGTCTGCCATCC | 2769 | GGATGGCAGACTGTGTGCT |
| siRNA 1041 | 1041 | CATCCCATATGCATTAAGA | 2783 | TCTTAATGCATATGGGATG |
| siRNA 1046 | 1046 | CATATGCATTAAGAGTGGA | 2788 | TCCACTCTTAATGCATATG |
| siRNA 1047 | 1047 | ATATGCATTAAGAGTGGAA | 2789 | TTCCACTCTTAATGCATAT |
| siRNA 1052 | 1052 | CATTAAGAGTGGAACTGGA | 2794 | TCCAGTTCCACTCTTAATG |
| siRNA 1060 | 1060 | GTGGAACTGGAAGACTGGA | 2802 | TCCAGTCTTCCAGTTCCAC |
| siRNA 1071 | 1071 | AGACTGGAATGGCAGAACC | 2813 | GGTTCTGCCATTCCAGTCT |
| siRNA 1079 | 1079 | ATGGCAGAACCAGTACTGC | 2821 | GCAGTACTGGTTCTGCCAT |
| siRNA 1085 | 1085 | GAACCAGTACTGCAGACTA | 2827 | TAGTCTGCAGTACTGGTTC |
| siRNA 1088 | 1088 | CCAGTACTGCAGACTATGC | 2830 | GCATAGTCTGCAGTACTGG |
| siRNA 1089 | 1089 | CAGTACTGCAGACTATGCC | 2831 | GGCATAGTCTGCAGTACTG |
| siRNA 1091 | 1091 | GTACTGCAGACTATGCCAT | 2833 | ATGGCATAGTCTGCAGTAC |
| siRNA 1095 | 1095 | TGCAGACTATGCCATGTTC | 2837 | GAACATGGCATAGTCTGCA |
| siRNA 1110 | 1110 | GTTCAAGGTGGGACCTGAA | 2852 | TTCAGGTCCCACCTTGAAC |
| siRNA 1111 | 1111 | TTCAAGGTGGGACCTGAAG | 2853 | CTTCAGGTCCCACCTTGAA |
| siRNA 1120 | 1120 | GGACCTGAAGCTGACAAGT | 2862 | ACTTGTCAGCTTCAGGTCC |
| siRNA 1123 | 1123 | CCTGAAGCTGACAAGTACC | 2865 | GGTACTTGTCAGCTTCAGG |
| siRNA 1126 | 1126 | GAAGCTGACAAGTACCGCC | 2868 | GGCGGTACTTGTCAGCTTC |
| siRNA 1127 | 1127 | AAGCTGACAAGTACCGCCT | 2869 | AGGCGGTACTTGTCAGCTT |
| siRNA 1128 | 1128 | AGCTGACAAGTACCGCCTA | 2870 | TAGGCGGTACTTGTCAGCT |
| siRNA 1129 | 1129 | GCTGACAAGTACCGCCTAA | 2871 | TTAGGCGGTACTTGTCAGC |
| siRNA 1130 | 1130 | CTGACAAGTACCGCCTAAC | 2872 | GTTAGGCGGTACTTGTCAG |
| siRNA 1131 | 1131 | TGACAAGTACCGCCTAACA | 2873 | TGTTAGGCGGTACTTGTCA |
| siRNA 1132 | 1132 | GACAAGTACCGCCTAACAT | 2874 | ATGTTAGGCGGTACTTGTC |
| siRNA 1133 | 1133 | ACAAGTACCGCCTAACATA | 2875 | TATGTTAGGCGGTACTTGT |
| siRNA 1134 | 1134 | CAAGTACCGCCTAACATAT | 2876 | ATATGTTAGGCGGTACTTG |
| siRNA 1135 | 1135 | AAGTACCGCCTAACATATG | 2877 | CATATGTTAGGCGGTACTT |
| siRNA 1136 | 1136 | AGTACCGCCTAACATATGC | 2878 | GCATATGTTAGGCGGTACT |
| siRNA 1137 | 1137 | GTACCGCCTAACATATGCC | 2879 | GGCATATGTTAGGCGGTAC |
| siRNA 1138 | 1138 | TACCGCCTAACATATGCCT | 2880 | AGGCATATGTTAGGCGGTA |
| siRNA 1139 | 1139 | ACCGCCTAACATATGCCTA | 2881 | TAGGCATATGTTAGGCGGT |
| siRNA 1140 | 1140 | CCGCCTAACATATGCCTAC | 2882 | GTAGGCATATGTTAGGCGG |
| siRNA 1141 | 1141 | CGCCTAACATATGCCTACT | 2883 | AGTAGGCATATGTTAGGCG |
| siRNA 1143 | 1143 | CCTAACATATGCCTACTTC | 2885 | GAAGTAGGCATATGTTAGG |
| siRNA 1146 | 1146 | AACATATGCCTACTTCGCT | 2888 | AGCGAAGTAGGCATATGTT |
| siRNA 1147 | 1147 | ACATATGCCTACTTCGCTG | 2889 | CAGCGAAGTAGGCATATGT |
| siRNA 1148 | 1148 | CATATGCCTACTTCGCTGG | 2890 | CCAGCGAAGTAGGCATATG |
| siRNA 1149 | 1149 | ATATGCCTACTTCGCTGGT | 2891 | ACCAGCGAAGTAGGCATAT |
| siRNA 1151 | 1151 | ATGCCTACTTCGCTGGTGG | 2893 | CCACCAGCGAAGTAGGCAT |
| siRNA 1174 | 1174 | GCTGGAGATGCCTTTGATG | 2916 | CATCAAAGGCATCTCCAGC |
| siRNA 1178 | 1178 | GAGATGCCTTTGATGGCTT | 2920 | AAGCCATCAAAGGCATCTC |
| siRNA 1182 | 1182 | TGCCTTTGATGGCTTTGAT | 2924 | ATCAAAGCCATCAAAGGCA |
| siRNA 1197 | 1197 | TGATTTTGGCGATGATCCT | 2939 | AGGATCATCGCCAAAATCA |
| siRNA 1198 | 1198 | GATTTTGGCGATGATCCTA | 2940 | TAGGATCATCGCCAAAATC |
| siRNA 1203 | 1203 | TGGCGATGATCCTAGTGAC | 2945 | GTCACTAGGATCATCGCCA |
| siRNA 1205 | 1205 | GCGATGATCCTAGTGACAA | 2947 | TTGTCACTAGGATCATCGC |
| siRNA 1206 | 1206 | CGATGATCCTAGTGACAAG | 2948 | CTTGTCACTAGGATCATCG |
| siRNA 1215 | 1215 | TAGTGACAAGTTTTTCACA | 2957 | TGTGAAAAACTTGTCACTA |
| siRNA 1227 | 1227 | TTTCACATCCCATAATGGC | 2969 | GCCATTATGGGATGTGAAA |
| siRNA 1236 | 1236 | CCATAATGGCATGCAGTTC | 2978 | GAACTGCATGCCATTATGG |
| siRNA 1242 | 1242 | TGGCATGCAGTTCAGTACC | 2984 | GGTACTGAACTGCATGCCA |
| siRNA 1243 | 1243 | GGCATGCAGTTCAGTACCT | 2985 | AGGTACTGAACTGCATGCC |
| siRNA 1248 | 1248 | GCAGTTCAGTACCTGGGAC | 2990 | GTCCCAGGTACTGAACTGC |
| siRNA 1249 | 1249 | CAGTTCAGTACCTGGGACA | 2991 | TGTCCCAGGTACTGAACTG |
| siRNA 1264 | 1264 | GACAATGACAATGATAAGT | 3006 | ACTTATCATTGTCATTGTC |
| siRNA 1265 | 1265 | ACAATGACAATGATAAGTT | 3007 | AACTTATCATTGTCATTGT |
| siRNA 1278 | 1278 | TAAGTTTGAAGGCAACTGT | 3020 | ACAGTTGCCTTCAAACTTA |
| siRNA 1286 | 1286 | AAGGCAACTGTGCTGAACA | 3028 | TGTTCAGCACAGTTGCCTT |
| siRNA 1287 | 1287 | AGGCAACTGTGCTGAACAG | 3029 | CTGTTCAGCACAGTTGCCT |
| siRNA 1298 | 1298 | CTGAACAGGATGGATCTGG | 3040 | CCAGATCCATCCTGTTCAG |
| siRNA 1304 | 1304 | AGGATGGATCTGGTTGGTG | 3046 | CACCAACCAGATCCATCCT |
| siRNA 1311 | 1311 | ATCTGGTTGGTGGATGAAC | 3053 | GTTCATCCACCAACCAGAT |
| siRNA 1315 | 1315 | GGTTGGTGGATGAACAAGT | 3057 | ACTTGTTCATCCACCAACC |
| siRNA 1319 | 1319 | GGTGGATGAACAAGTGTCA | 3061 | TGACACTTGTTCATCCACC |
| siRNA 1320 | 1320 | GTGGATGAACAAGTGTCAC | 3062 | GTGACACTTGTTCATCCAC |
| siRNA 1323 | 1323 | GATGAACAAGTGTCACGCT | 3065 | AGCGTGACACTTGTTCATC |
| siRNA 1324 | 1324 | ATGAACAAGTGTCACGCTG | 3066 | CAGCGTGACACTTGTTCAT |
| siRNA 1325 | 1325 | TGAACAAGTGTCACGCTGG | 3067 | CCAGCGTGACACTTGTTCA |
| siRNA 1326 | 1326 | GAACAAGTGTCACGCTGGC | 3068 | GCCAGCGTGACACTTGTTC |
| siRNA 1339 | 1339 | GCTGGCCATCTCAATGGAG | 3081 | CTCCATTGAGATGGCCAGC |
| siRNA 1342 | 1342 | GGCCATCTCAATGGAGTTT | 3084 | AAACTCCATTGAGATGGCC |
| siRNA 1344 | 1344 | CCATCTCAATGGAGTTTAT | 3086 | ATAAACTCCATTGAGATGG |
| siRNA 1348 | 1348 | CTCAATGGAGTTTATTACC | 3090 | GGTAATAAACTCCATTGAG |
| siRNA 1349 | 1349 | TCAATGGAGTTTATTACCA | 3091 | TGGTAATAAACTCCATTGA |
| siRNA 1354 | 1354 | GGAGTTTATTACCAAGGTG | 3096 | CACCTTGGTAATAAACTCC |
| siRNA 1356 | 1356 | AGTTTATTACCAAGGTGGC | 3098 | GCCACCTTGGTAATAAACT |
| siRNA 1359 | 1359 | TTATTACCAAGGTGGCACT | 3101 | AGTGCCACCTTGGTAATAA |
| siRNA 1362 | 1362 | TTACCAAGGTGGCACTTAC | 3104 | GTAAGTGCCACCTTGGTAA |
| siRNA 1365 | 1365 | CCAAGGTGGCACTTACTCA | 3107 | TGAGTAAGTGCCACCTTGG |
| siRNA 1368 | 1368 | AGGTGGCACTTACTCAAAA | 3110 | TTTTGAGTAAGTGCCACCT |
| siRNA 1374 | 1374 | CACTTACTCAAAAGCATCT | 3116 | AGATGCTTTTGAGTAAGTG |
| siRNA 1383 | 1383 | AAAAGCATCTACTCCTAAT | 3125 | ATTAGGAGTAGATGCTTTT |
| siRNA 1384 | 1384 | AAAGCATCTACTCCTAATG | 3126 | CATTAGGAGTAGATGCTTT |
| siRNA 1385 | 1385 | AAGCATCTACTCCTAATGG | 3127 | CCATTAGGAGTAGATGCTT |
| siRNA 1386 | 1386 | AGCATCTACTCCTAATGGT | 3128 | ACCATTAGGAGTAGATGCT |
| siRNA 1387 | 1387 | GCATCTACTCCTAATGGTT | 3129 | AACCATTAGGAGTAGATGC |
| siRNA 1389 | 1389 | ATCTACTCCTAATGGTTAT | 3131 | ATAACCATTAGGAGTAGAT |
| siRNA 1390 | 1390 | TCTACTCCTAATGGTTATG | 3132 | CATAACCATTAGGAGTAGA |
| siRNA 1391 | 1391 | CTACTCCTAATGGTTATGA | 3133 | TCATAACCATTAGGAGTAG |
| siRNA 1394 | 1394 | CTCCTAATGGTTATGATAA | 3136 | TTATCATAACCATTAGGAG |
| siRNA 1395 | 1395 | TCCTAATGGTTATGATAAT | 3137 | ATTATCATAACCATTAGGA |
| siRNA 1396 | 1396 | CCTAATGGTTATGATAATG | 3138 | CATTATCATAACCATTAGG |
| siRNA 1401 | 1401 | TGGTTATGATAATGGCATT | 3143 | AATGCCATTATCATAACCA |
| siRNA 1404 | 1404 | TTATGATAATGGCATTATT | 3146 | AATAATGCCATTATCATAA |
| siRNA 1409 | 1409 | ATAATGGCATTATTTGGGC | 3151 | GCCCAAATAATGCCATTAT |
| siRNA 1410 | 1410 | TAATGGCATTATTTGGGCC | 3152 | GGCCCAAATAATGCCATTA |
| siRNA 1417 | 1417 | ATTATTTGGGCCACTTGGA | 3159 | TCCAAGTGGCCCAAATAAT |
| siRNA 1418 | 1418 | TTATTTGGGCCACTTGGAA | 3160 | TTCCAAGTGGCCCAAATAA |
| siRNA 1419 | 1419 | TATTTGGGCCACTTGGAAA | 3161 | TTTCCAAGTGGCCCAAATA |
| siRNA 1420 | 1420 | ATTTGGGCCACTTGGAAAA | 3162 | TTTTCCAAGTGGCCCAAAT |
| siRNA 1426 | 1426 | GCCACTTGGAAAACCCGGT | 3168 | ACCGGGTTTTCCAAGTGGC |
| siRNA 1427 | 1427 | CCACTTGGAAAACCCGGTG | 3169 | CACCGGGTTTTCCAAGTGG |
| siRNA 1428 | 1428 | CACTTGGAAAACCCGGTGG | 3170 | CCACCGGGTTTTCCAAGTG |
| siRNA 1431 | 1431 | TTGGAAAACCCGGTGGTAT | 3173 | ATACCACCGGGTTTTCCAA |
| siRNA 1432 | 1432 | TGGAAAACCCGGTGGTATT | 3174 | AATACCACCGGGTTTTCCA |
| siRNA 1434 | 1434 | GAAAACCCGGTGGTATTCC | 3176 | GGAATACCACCGGGTTTTC |
| siRNA 1435 | 1435 | AAAACCCGGTGGTATTCCA | 3177 | TGGAATACCACCGGGTTTT |
| siRNA 1440 | 1440 | CCGGTGGTATTCCATGAAG | 3182 | CTTCATGGAATACCACCGG |
| siRNA 1461 | 1461 | AACCACTATGAAGATAATC | 3203 | GATTATCTTCATAGTGGTT |
| siRNA 1462 | 1462 | ACCACTATGAAGATAATCC | 3204 | GGATTATCTTCATAGTGGT |
| siRNA 1466 | 1466 | CTATGAAGATAATCCCATT | 3208 | AATGGGATTATCTTCATAG |
| siRNA 1467 | 1467 | TATGAAGATAATCCCATTC | 3209 | GAATGGGATTATCTTCATA |
| siRNA 1477 | 1477 | ATCCCATTCAACAGACTCA | 3219 | TGAGTCTGTTGAATGGGAT |
| siRNA 1478 | 1478 | TCCCATTCAACAGACTCAC | 3220 | GTGAGTCTGTTGAATGGGA |
| siRNA 1482 | 1482 | ATTCAACAGACTCACAATT | 3224 | AATTGTGAGTCTGTTGAAT |
| siRNA 1485 | 1485 | CAACAGACTCACAATTGGA | 3227 | TCCAATTGTGAGTCTGTTG |
| siRNA 1487 | 1487 | ACAGACTCACAATTGGAGA | 3229 | TCTCCAATTGTGAGTCTGT |
| siRNA 1488 | 1488 | CAGACTCACAATTGGAGAA | 3230 | TTCTCCAATTGTGAGTCTG |
| siRNA 1519 | 1519 | CACCTGGGGGGAGCCAAAC | 3261 | GTTTGGCTCCCCCCAGGTG |
| siRNA 1533 | 1533 | CAAACAGGCTGGAGACGTT | 3275 | AACGTCTCCAGCCTGTTTG |
| siRNA 1540 | 1540 | GCTGGAGACGTTTAAAAGA | 3282 | TCTTTTAAACGTCTCCAGC |
| siRNA 1583 | 1583 | TTAAAGGACTTTATCTGAA | 3325 | TTCAGATAAAGTCCTTTAA |
| siRNA 1588 | 1588 | GGACTTTATCTGAACAGAG | 3330 | CTCTGTTCAGATAAAGTCC |
| siRNA 1592 | 1592 | TTTATCTGAACAGAGAGAT | 3334 | ATCTCTCTGTTCAGATAAA |
| siRNA 1597 | 1597 | CTGAACAGAGAGATATAAT | 3339 | ATTATATCTCTCTGTTCAG |
| siRNA 1615 | 1615 | TATTTTTCCTATTGGACAA | 3357 | TTGTCCAATAGGAAAAATA |
| siRNA 1617 | 1617 | TTTTTCCTATTGGACAATG | 3359 | CATTGTCCAATAGGAAAAA |
| siRNA 1622 | 1622 | CCTATTGGACAATGGACTT | 3364 | AAGTCCATTGTCCAATAGG |
| siRNA 1623 | 1623 | CTATTGGACAATGGACTTG | 3365 | CAAGTCCATTGTCCAATAG |
| siRNA 1626 | 1626 | TTGGACAATGGACTTGCAA | 3368 | TTGCAAGTCCATTGTCCAA |
| siRNA 1627 | 1627 | TGGACAATGGACTTGCAAA | 3369 | TTTGCAAGTCCATTGTCCA |
| siRNA 1647 | 1647 | CTTCACTTCATTTTAAGAG | 3389 | CTCTTAAAATGAAGTGAAG |
| siRNA 1648 | 1648 | TTCACTTCATTTTAAGAGC | 3390 | GCTCTTAAAATGAAGTGAA |
| siRNA 1649 | 1649 | TCACTTCATTTTAAGAGCA | 3391 | TGCTCTTAAAATGAAGTGA |
| siRNA 1664 | 1664 | AGCAAAAGACCCCATGTTG | 3406 | CAACATGGGGTCTTTTGCT |
| siRNA 1672 | 1672 | ACCCCATGTTGAAAACTCC | 3414 | GGAGTTTTCAACATGGGGT |
| siRNA 1678 | 1678 | TGTTGAAAACTCCATAACA | 3420 | TGTTATGGAGTTTTCAACA |
| siRNA 1679 | 1679 | GTTGAAAACTCCATAACAG | 3421 | CTGTTATGGAGTTTTCAAC |
| siRNA 1680 | 1680 | TTGAAAACTCCATAACAGT | 3422 | ACTGTTATGGAGTTTTCAA |
| siRNA 1691 | 1691 | ATAACAGTTTTATGCTGAT | 3433 | ATCAGCATAAAACTGTTAT |
| siRNA 1696 | 1696 | AGTTTTATGCTGATGATAA | 3438 | TTATCATCAGCATAAAACT |
| siRNA 1700 | 1700 | TTATGCTGATGATAATTTA | 3442 | TAAATTATCATCAGCATAA |
| siRNA 1710 | 1710 | GATAATTTATCTACATGCA | 3452 | TGCATGTAGATAAATTATC |

The siRNAs in subset B were selected to have the following characteristics:
Cross-reactivity: With 19mer in human FGG mRNA, with 17mer/19mer in NHP FGG
Specificity category: For human and NHP: AS2 or better, SS3 or better
miRNA seeds: AS±SS strand: seed region not conserved in human, mouse, and rat and not present in >4 species
Off-target frequency: ≤20 human off-targets matched with 2 mismatches in antisense strand
SNPs: siRNA target sites do not harbor SNPs with a MAF≥1% (pos. 2-18)

The siRNA sequences in subset B were further selected for absence of seed regions in the AS strand that are identical to a seed region of known human miRNA to yield subset C. Subset C includes 221 siRNAs whose base sequences are shown in Table 5.

TABLE 5
Subset C siRNAs

| | Sense | | Antisense | |
|---|---|---|---|---|
| | strand | | strand | |
| | SEQ | Sense strand sequence | SEQ | Antisense strand sequence |
| siRNA Name | ID NO: | (5′-3′) | ID NO: | (5′-3′) |
| siRNA 224 | 224 | CCGGGCACTCAGACATCAT | 1966 | ATGATGTCTGAGTGCCCGG |
| siRNA 233 | 233 | CAGACATCATGAGTTGGTC | 1975 | GACCAACTCATGATGTCTG |
| siRNA 234 | 234 | AGACATCATGAGTTGGTCC | 1976 | GGACCAACTCATGATGTCT |
| siRNA 250 | 250 | TCCTTGCACCCCCGGAATT | 1992 | AATTCCGGGGGTGCAAGGA |
| siRNA 251 | 251 | CCTTGCACCCCCGGAATTT | 1993 | AAATTCCGGGGGTGCAAGG |
| siRNA 253 | 253 | TTGCACCCCCGGAATTTAA | 1995 | TTAAATTCCGGGGGTGCAA |
| siRNA 254 | 254 | TGCACCCCCGGAATTTAAT | 1996 | ATTAAATTCCGGGGGTGCA |
| siRNA 255 | 255 | GCACCCCCGGAATTTAATT | 1997 | AATTAAATTCCGGGGGTGC |
| siRNA 258 | 258 | CCCCCGGAATTTAATTCTC | 2000 | GAGAATTAAATTCCGGGGG |
| siRNA 261 | 261 | CCGGAATTTAATTCTCTAC | 2003 | GTAGAGAATTAAATTCCGG |
| siRNA 269 | 269 | TAATTCTCTACTTCTATGC | 2011 | GCATAGAAGTAGAGAATTA |
| siRNA 274 | 274 | CTCTACTTCTATGCTCTTT | 2016 | AAAGAGCATAGAAGTAGAG |
| siRNA 310 | 310 | TGTGTAGCATATGTTGCTA | 2052 | TAGCAACATATGCTACACA |
| siRNA 323 | 323 | TTGCTACCAGAGACAACTG | 2065 | CAGTTGTCTCTGGTAGCAA |
| siRNA 324 | 324 | TGCTACCAGAGACAACTGC | 2066 | GCAGTTGTCTCTGGTAGCA |
| siRNA 335 | 335 | ACAACTGCTGCATCTTAGA | 2077 | TCTAAGATGCAGCAGTTGT |
| siRNA 342 | 342 | CTGCATCTTAGATGAAAGA | 2084 | TCTTTCATCTAAGATGCAG |
| siRNA 343 | 343 | TGCATCTTAGATGAAAGAT | 2085 | ATCTTTCATCTAAGATGCA |
| siRNA 344 | 344 | GCATCTTAGATGAAAGATT | 2086 | AATCTTTCATCTAAGATGC |
| siRNA 347 | 347 | TCTTAGATGAAAGATTCGG | 2089 | CCGAATCTTTCATCTAAGA |
| siRNA 348 | 348 | CTTAGATGAAAGATTCGGT | 2090 | ACCGAATCTTTCATCTAAG |
| siRNA 349 | 349 | TTAGATGAAAGATTCGGTA | 2091 | TACCGAATCTTTCATCTAA |
| siRNA 350 | 350 | TAGATGAAAGATTCGGTAG | 2092 | CTACCGAATCTTTCATCTA |
| siRNA 351 | 351 | AGATGAAAGATTCGGTAGT | 2093 | ACTACCGAATCTTTCATCT |
| siRNA 352 | 352 | GATGAAAGATTCGGTAGTT | 2094 | AACTACCGAATCTTTCATC |
| siRNA 354 | 354 | TGAAAGATTCGGTAGTTAT | 2096 | ATAACTACCGAATCTTTCA |
| siRNA 355 | 355 | GAAAGATTCGGTAGTTATT | 2097 | AATAACTACCGAATCTTTC |
| siRNA 359 | 359 | GATTCGGTAGTTATTGTCC | 2101 | GGACAATAACTACCGAATC |
| siRNA 363 | 363 | CGGTAGTTATTGTCCAACT | 2105 | AGTTGGACAATAACTACCG |
| siRNA 365 | 365 | GTAGTTATTGTCCAACTAC | 2107 | GTAGTTGGACAATAACTAC |
| siRNA 367 | 367 | AGTTATTGTCCAACTACCT | 2109 | AGGTAGTTGGACAATAACT |
| siRNA 369 | 369 | TTATTGTCCAACTACCTGT | 2111 | ACAGGTAGTTGGACAATAA |
| siRNA 372 | 372 | TTGTCCAACTACCTGTGGC | 2114 | GCCACAGGTAGTTGGACAA |
| siRNA 373 | 373 | TGTCCAACTACCTGTGGCA | 2115 | TGCCACAGGTAGTTGGACA |
| siRNA 384 | 384 | CTGTGGCATTGCAGATTTC | 2126 | GAAATCTGCAATGCCACAG |
| siRNA 392 | 392 | TTGCAGATTTCCTGTCTAC | 2134 | GTAGACAGGAAATCTGCAA |
| siRNA 397 | 397 | GATTTCCTGTCTACTTATC | 2139 | GATAAGTAGACAGGAAATC |
| siRNA 398 | 398 | ATTTCCTGTCTACTTATCA | 2140 | TGATAAGTAGACAGGAAAT |
| siRNA 406 | 406 | TCTACTTATCAAACCAAAG | 2148 | CTTTGGTTTGATAAGTAGA |
| siRNA 411 | 411 | TTATCAAACCAAAGTAGAC | 2153 | GTCTACTTTGGTTTGATAA |
| siRNA 423 | 423 | AGTAGACAAGGATCTACAG | 2165 | CTGTAGATCCTTGTCTACT |
| siRNA 426 | 426 | AGACAAGGATCTACAGTCT | 2168 | AGACTGTAGATCCTTGTCT |
| siRNA 432 | 432 | GGATCTACAGTCTTTGGAA | 2174 | TTCCAAAGACTGTAGATCC |
| siRNA 434 | 434 | ATCTACAGTCTTTGGAAGA | 2176 | TCTTCCAAAGACTGTAGAT |
| siRNA 447 | 447 | GGAAGACATCTTACATCAA | 2189 | TTGATGTAAGATGTCTTCC |
| siRNA 449 | 449 | AAGACATCTTACATCAAGT | 2191 | ACTTGATGTAAGATGTCTT |
| siRNA 450 | 450 | AGACATCTTACATCAAGTT | 2192 | AACTTGATGTAAGATGTCT |
| siRNA 493 | 493 | CAGCTGATAAAAGCAATCC | 2235 | GGATTGCTTTTATCAGCTG |
| siRNA 494 | 494 | AGCTGATAAAAGCAATCCA | 2236 | TGGATTGCTTTTATCAGCT |
| siRNA 497 | 497 | TGATAAAAGCAATCCAACT | 2239 | AGTTGGATTGCTTTTATCA |
| siRNA 504 | 504 | AGCAATCCAACTCACTTAT | 2246 | ATAAGTGAGTTGGATTGCT |
| siRNA 505 | 505 | GCAATCCAACTCACTTATA | 2247 | TATAAGTGAGTTGGATTGC |
| siRNA 507 | 507 | AATCCAACTCACTTATAAT | 2249 | ATTATAAGTGAGTTGGATT |
| siRNA 508 | 508 | ATCCAACTCACTTATAATC | 2250 | GATTATAAGTGAGTTGGAT |
| siRNA 509 | 509 | TCCAACTCACTTATAATCC | 2251 | GGATTATAAGTGAGTTGGA |
| siRNA 510 | 510 | CCAACTCACTTATAATCCT | 2252 | AGGATTATAAGTGAGTTGG |
| siRNA 514 | 514 | CTCACTTATAATCCTGATG | 2256 | CATCAGGATTATAAGTGAG |
| siRNA 515 | 515 | TCACTTATAATCCTGATGA | 2257 | TCATCAGGATTATAAGTGA |
| siRNA 523 | 523 | AATCCTGATGAATCATCAA | 2265 | TTGATGATTCATCAGGATT |
| siRNA 528 | 528 | TGATGAATCATCAAAACCA | 2270 | TGGTTTTGATGATTCATCA |
| siRNA 539 | 539 | CAAAACCAAATATGATAGA | 2281 | TCTATCATATTTGGTTTTG |
| siRNA 544 | 544 | CCAAATATGATAGACGCTG | 2286 | CAGCGTCTATCATATTTGG |
| siRNA 546 | 546 | AAATATGATAGACGCTGCT | 2288 | AGCAGCGTCTATCATATTT |
| siRNA 554 | 554 | TAGACGCTGCTACTTTGAA | 2296 | TTCAAAGTAGCAGCGTCTA |
| siRNA 573 | 573 | GTCCAGGAAAATGTTAGAA | 2315 | TTCTAACATTTTCCTGGAC |
| siRNA 599 | 599 | TGAAATATGAAGCATCGAT | 2341 | ATCGATGCTTCATATTTCA |
| siRNA 600 | 600 | GAAATATGAAGCATCGATT | 2342 | AATCGATGCTTCATATTTC |
| siRNA 601 | 601 | AAATATGAAGCATCGATTT | 2343 | AAATCGATGCTTCATATTT |
| siRNA 604 | 604 | TATGAAGCATCGATTTTAA | 2346 | TTAAAATCGATGCTTCATA |
| siRNA 606 | 606 | TGAAGCATCGATTTTAACA | 2348 | TGTTAAAATCGATGCTTCA |
| siRNA 609 | 609 | AGCATCGATTTTAACACAT | 2351 | ATGTGTTAAAATCGATGCT |
| siRNA 619 | 619 | TTAACACATGACTCAAGTA | 2361 | TACTTGAGTCATGTGTTAA |
| siRNA 624 | 624 | ACATGACTCAAGTATTCGA | 2366 | TCGAATACTTGAGTCATGT |
| siRNA 625 | 625 | CATGACTCAAGTATTCGAT | 2367 | ATCGAATACTTGAGTCATG |
| siRNA 661 | 661 | AATTCAAATAATCAAAAGA | 2403 | TCTTTTGATTATTTGAATT |
| siRNA 716 | 716 | CACAGTGCCAGGAACCTTG | 2458 | CAAGGTTCCTGGCACTGTG |
| siRNA 720 | 720 | GTGCCAGGAACCTTGCAAA | 2462 | TTTGCAAGGTTCCTGGCAC |
| siRNA 728 | 728 | AACCTTGCAAAGACACGGT | 2470 | ACCGTGTCTTTGCAAGGTT |
| siRNA 729 | 729 | ACCTTGCAAAGACACGGTG | 2471 | CACCGTGTCTTTGCAAGGT |
| siRNA 730 | 730 | CCTTGCAAAGACACGGTGC | 2472 | GCACCGTGTCTTTGCAAGG |
| siRNA 731 | 731 | CTTGCAAAGACACGGTGCA | 2473 | TGCACCGTGTCTTTGCAAG |
| siRNA 733 | 733 | TGCAAAGACACGGTGCAAA | 2475 | TTTGCACCGTGTCTTTGCA |
| siRNA 736 | 736 | AAAGACACGGTGCAAATCC | 2478 | GGATTTGCACCGTGTCTTT |
| siRNA 751 | 751 | ATCCATGATATCACTGGGA | 2493 | TCCCAGTGATATCATGGAT |
| siRNA 753 | 753 | CCATGATATCACTGGGAAA | 2495 | TTTCCCAGTGATATCATGG |
| siRNA 770 | 770 | AAGATTGTCAAGACATTGC | 2512 | GCAATGTCTTGACAATCTT |
| siRNA 778 | 778 | CAAGACATTGCCAATAAGG | 2520 | CCTTATTGGCAATGTCTTG |
| siRNA 780 | 780 | AGACATTGCCAATAAGGGA | 2522 | TCCCTTATTGGCAATGTCT |
| siRNA 784 | 784 | ATTGCCAATAAGGGAGCTA | 2526 | TAGCTCCCTTATTGGCAAT |
| siRNA 785 | 785 | TTGCCAATAAGGGAGCTAA | 2527 | TTAGCTCCCTTATTGGCAA |
| siRNA 786 | 786 | TGCCAATAAGGGAGCTAAA | 2528 | TTTAGCTCCCTTATTGGCA |
| siRNA 791 | 791 | ATAAGGGAGCTAAACAGAG | 2533 | CTCTGTTTAGCTCCCTTAT |
| siRNA 795 | 795 | GGGAGCTAAACAGAGCGGG | 2537 | CCCGCTCTGTTTAGCTCCC |
| siRNA 796 | 796 | GGAGCTAAACAGAGCGGGC | 2538 | GCCCGCTCTGTTTAGCTCC |
| siRNA 800 | 800 | CTAAACAGAGCGGGCTTTA | 2542 | TAAAGCCCGCTCTGTTTAG |
| siRNA 802 | 802 | AAACAGAGCGGGCTTTACT | 2544 | AGTAAAGCCCGCTCTGTTT |
| siRNA 806 | 806 | AGAGCGGGCTTTACTTTAT | 2548 | ATAAAGTAAAGCCCGCTCT |
| siRNA 812 | 812 | GGCTTTACTTTATTAAACC | 2554 | GGTTTAATAAAGTAAAGCC |
| siRNA 821 | 821 | TTATTAAACCTCTGAAAGC | 2563 | GCTTTCAGAGGTTTAATAA |
| siRNA 825 | 825 | TAAACCTCTGAAAGCTAAC | 2567 | GTTAGCTTTCAGAGGTTTA |
| siRNA 826 | 826 | AAACCTCTGAAAGCTAACC | 2568 | GGTTAGCTTTCAGAGGTTT |
| siRNA 827 | 827 | AACCTCTGAAAGCTAACCA | 2569 | TGGTTAGCTTTCAGAGGTT |
| siRNA 833 | 833 | TGAAAGCTAACCAGCAATT | 2575 | AATTGCTGGTTAGCTTTCA |
| siRNA 844 | 844 | CAGCAATTCTTAGTCTACT | 2586 | AGTAGACTAAGAATTGCTG |
| siRNA 850 | 850 | TTCTTAGTCTACTGTGAAA | 2592 | TTTCACAGTAGACTAAGAA |
| siRNA 877 | 877 | TCTGGAAATGGATGGACTG | 2619 | CAGTCCATCCATTTCCAGA |
| siRNA 894 | 894 | TGTGTTTCAGAAGAGACTT | 2636 | AAGTCTCTTCTGAAACACA |
| siRNA 904 | 904 | AAGAGACTTGATGGCAGTG | 2646 | CACTGCCATCAAGTCTCTT |
| siRNA 929 | 929 | TCAAGAAAAACTGGATTCA | 2671 | TGAATCCAGTTTTTCTTGA |
| siRNA 932 | 932 | AGAAAAACTGGATTCAATA | 2674 | TATTGAATCCAGTTTTTCT |
| siRNA 964 | 964 | GGACATCTGTCTCCTACTG | 2706 | CAGTAGGAGACAGATGTCC |
| siRNA 972 | 972 | GTCTCCTACTGGCACAACA | 2714 | TGTTGTGCCAGTAGGAGAC |
| siRNA 973 | 973 | TCTCCTACTGGCACAACAG | 2715 | CTGTTGTGCCAGTAGGAGA |
| siRNA 983 | 983 | GCACAACAGAATTTTGGCT | 2725 | AGCCAAAATTCTGTTGTGC |
| siRNA 1003 | 1003 | GGAAATGAGAAGATTCATT | 2745 | AATGAATCTTCTCATTTCC |
| siRNA 1011 | 1011 | GAAGATTCATTTGATAAGC | 2753 | GCTTATCAAATGAATCTTC |
| siRNA 1013 | 1013 | AGATTCATTTGATAAGCAC | 2755 | GTGCTTATCAAATGAATCT |
| siRNA 1024 | 1024 | ATAAGCACACAGTCTGCCA | 2766 | TGGCAGACTGTGTGCTTAT |
| siRNA 1027 | 1027 | AGCACACAGTCTGCCATCC | 2769 | GGATGGCAGACTGTGTGCT |
| siRNA 1041 | 1041 | CATCCCATATGCATTAAGA | 2783 | TCTTAATGCATATGGGATG |
| siRNA 1052 | 1052 | CATTAAGAGTGGAACTGGA | 2794 | TCCAGTTCCACTCTTAATG |
| siRNA 1060 | 1060 | GTGGAACTGGAAGACTGGA | 2802 | TCCAGTCTTCCAGTTCCAC |
| siRNA 1071 | 1071 | AGACTGGAATGGCAGAACC | 2813 | GGTTCTGCCATTCCAGTCT |
| siRNA 1079 | 1079 | ATGGCAGAACCAGTACTGC | 2821 | GCAGTACTGGTTCTGCCAT |
| siRNA 1085 | 1085 | GAACCAGTACTGCAGACTA | 2827 | TAGTCTGCAGTACTGGTTC |
| siRNA 1088 | 1088 | CCAGTACTGCAGACTATGC | 2830 | GCATAGTCTGCAGTACTGG |
| siRNA 1089 | 1089 | CAGTACTGCAGACTATGCC | 2831 | GGCATAGTCTGCAGTACTG |
| siRNA 1095 | 1095 | TGCAGACTATGCCATGTTC | 2837 | GAACATGGCATAGTCTGCA |
| siRNA 1111 | 1111 | TTCAAGGTGGGACCTGAAG | 2853 | CTTCAGGTCCCACCTTGAA |
| siRNA 1123 | 1123 | CCTGAAGCTGACAAGTACC | 2865 | GGTACTTGTCAGCTTCAGG |
| siRNA 1126 | 1126 | GAAGCTGACAAGTACCGCC | 2868 | GGCGGTACTTGTCAGCTTC |
| siRNA 1127 | 1127 | AAGCTGACAAGTACCGCCT | 2869 | AGGCGGTACTTGTCAGCTT |
| siRNA 1128 | 1128 | AGCTGACAAGTACCGCCTA | 2870 | TAGGCGGTACTTGTCAGCT |
| siRNA 1129 | 1129 | GCTGACAAGTACCGCCTAA | 2871 | TTAGGCGGTACTTGTCAGC |
| siRNA 1130 | 1130 | CTGACAAGTACCGCCTAAC | 2872 | GTTAGGCGGTACTTGTCAG |
| siRNA 1132 | 1132 | GACAAGTACCGCCTAACAT | 2874 | ATGTTAGGCGGTACTTGTC |
| siRNA 1133 | 1133 | ACAAGTACCGCCTAACATA | 2875 | TATGTTAGGCGGTACTTGT |
| siRNA 1134 | 1134 | CAAGTACCGCCTAACATAT | 2876 | ATATGTTAGGCGGTACTTG |
| siRNA 1135 | 1135 | AAGTACCGCCTAACATATG | 2877 | CATATGTTAGGCGGTACTT |
| siRNA 1136 | 1136 | AGTACCGCCTAACATATGC | 2878 | GCATATGTTAGGCGGTACT |
| siRNA 1137 | 1137 | GTACCGCCTAACATATGCC | 2879 | GGCATATGTTAGGCGGTAC |
| siRNA 1138 | 1138 | TACCGCCTAACATATGCCT | 2880 | AGGCATATGTTAGGCGGTA |
| siRNA 1139 | 1139 | ACCGCCTAACATATGCCTA | 2881 | TAGGCATATGTTAGGCGGT |
| siRNA 1146 | 1146 | AACATATGCCTACTTCGCT | 2888 | AGCGAAGTAGGCATATGTT |
| siRNA 1147 | 1147 | ACATATGCCTACTTCGCTG | 2889 | CAGCGAAGTAGGCATATGT |
| siRNA 1148 | 1148 | CATATGCCTACTTCGCTGG | 2890 | CCAGCGAAGTAGGCATATG |
| siRNA 1174 | 1174 | GCTGGAGATGCCTTTGATG | 2916 | CATCAAAGGCATCTCCAGC |
| siRNA 1178 | 1178 | GAGATGCCTTTGATGGCTT | 2920 | AAGCCATCAAAGGCATCTC |
| siRNA 1182 | 1182 | TGCCTTTGATGGCTTTGAT | 2924 | ATCAAAGCCATCAAAGGCA |
| siRNA 1197 | 1197 | TGATTTTGGCGATGATCCT | 2939 | AGGATCATCGCCAAAATCA |
| siRNA 1203 | 1203 | TGGCGATGATCCTAGTGAC | 2945 | GTCACTAGGATCATCGCCA |
| siRNA 1205 | 1205 | GCGATGATCCTAGTGACAA | 2947 | TTGTCACTAGGATCATCGC |
| siRNA 1206 | 1206 | CGATGATCCTAGTGACAAG | 2948 | CTTGTCACTAGGATCATCG |
| siRNA 1215 | 1215 | TAGTGACAAGTTTTTCACA | 2957 | TGTGAAAAACTTGTCACTA |
| siRNA 1236 | 1236 | CCATAATGGCATGCAGTTC | 2978 | GAACTGCATGCCATTATGG |
| siRNA 1242 | 1242 | TGGCATGCAGTTCAGTACC | 2984 | GGTACTGAACTGCATGCCA |
| siRNA 1243 | 1243 | GGCATGCAGTTCAGTACCT | 2985 | AGGTACTGAACTGCATGCC |
| siRNA 1264 | 1264 | GACAATGACAATGATAAGT | 3006 | ACTTATCATTGTCATTGTC |
| siRNA 1265 | 1265 | ACAATGACAATGATAAGTT | 3007 | AACTTATCATTGTCATTGT |
| siRNA 1278 | 1278 | TAAGTTTGAAGGCAACTGT | 3020 | ACAGTTGCCTTCAAACTTA |
| siRNA 1286 | 1286 | AAGGCAACTGTGCTGAACA | 3028 | TGTTCAGCACAGTTGCCTT |
| siRNA 1287 | 1287 | AGGCAACTGTGCTGAACAG | 3029 | CTGTTCAGCACAGTTGCCT |
| siRNA 1304 | 1304 | AGGATGGATCTGGTTGGTG | 3046 | CACCAACCAGATCCATCCT |
| siRNA 1311 | 1311 | ATCTGGTTGGTGGATGAAC | 3053 | GTTCATCCACCAACCAGAT |
| siRNA 1315 | 1315 | GGTTGGTGGATGAACAAGT | 3057 | ACTTGTTCATCCACCAACC |
| siRNA 1320 | 1320 | GTGGATGAACAAGTGTCAC | 3062 | GTGACACTTGTTCATCCAC |
| siRNA 1323 | 1323 | GATGAACAAGTGTCACGCT | 3065 | AGCGTGACACTTGTTCATC |
| siRNA 1324 | 1324 | ATGAACAAGTGTCACGCTG | 3066 | CAGCGTGACACTTGTTCAT |
| siRNA 1325 | 1325 | TGAACAAGTGTCACGCTGG | 3067 | CCAGCGTGACACTTGTTCA |
| siRNA 1339 | 1339 | GCTGGCCATCTCAATGGAG | 3081 | CTCCATTGAGATGGCCAGC |
| siRNA 1342 | 1342 | GGCCATCTCAATGGAGTTT | 3084 | AAACTCCATTGAGATGGCC |
| siRNA 1344 | 1344 | CCATCTCAATGGAGTTTAT | 3086 | ATAAACTCCATTGAGATGG |
| siRNA 1348 | 1348 | CTCAATGGAGTTTATTACC | 3090 | GGTAATAAACTCCATTGAG |
| siRNA 1349 | 1349 | TCAATGGAGTTTATTACCA | 3091 | TGGTAATAAACTCCATTGA |
| siRNA 1356 | 1356 | AGTTTATTACCAAGGTGGC | 3098 | GCCACCTTGGTAATAAACT |
| siRNA 1362 | 1362 | TTACCAAGGTGGCACTTAC | 3104 | GTAAGTGCCACCTTGGTAA |
| siRNA 1365 | 1365 | CCAAGGTGGCACTTACTCA | 3107 | TGAGTAAGTGCCACCTTGG |
| siRNA 1374 | 1374 | CACTTACTCAAAAGCATCT | 3116 | AGATGCTTTTGAGTAAGTG |
| siRNA 1384 | 1384 | AAAGCATCTACTCCTAATG | 3126 | CATTAGGAGTAGATGCTTT |
| siRNA 1385 | 1385 | AAGCATCTACTCCTAATGG | 3127 | CCATTAGGAGTAGATGCTT |
| siRNA 1389 | 1389 | ATCTACTCCTAATGGTTAT | 3131 | ATAACCATTAGGAGTAGAT |
| siRNA 1390 | 1390 | TCTACTCCTAATGGTTATG | 3132 | CATAACCATTAGGAGTAGA |
| siRNA 1391 | 1391 | CTACTCCTAATGGTTATGA | 3133 | TCATAACCATTAGGAGTAG |
| siRNA 1394 | 1394 | CTCCTAATGGTTATGATAA | 3136 | TTATCATAACCATTAGGAG |
| siRNA 1396 | 1396 | CCTAATGGTTATGATAATG | 3138 | CATTATCATAACCATTAGG |
| siRNA 1401 | 1401 | TGGTTATGATAATGGCATT | 3143 | AATGCCATTATCATAACCA |
| siRNA 1404 | 1404 | TTATGATAATGGCATTATT | 3146 | AATAATGCCATTATCATAA |
| siRNA 1410 | 1410 | TAATGGCATTATTTGGGCC | 3152 | GGCCCAAATAATGCCATTA |
| siRNA 1418 | 1418 | TTATTTGGGCCACTTGGAA | 3160 | TTCCAAGTGGCCCAAATAA |
| siRNA 1419 | 1419 | TATTTGGGCCACTTGGAAA | 3161 | TTTCCAAGTGGCCCAAATA |
| siRNA 1420 | 1420 | ATTTGGGCCACTTGGAAAA | 3162 | TTTTCCAAGTGGCCCAAAT |
| siRNA 1426 | 1426 | GCCACTTGGAAAACCCGGT | 3168 | ACCGGGTTTTCCAAGTGGC |
| siRNA 1431 | 1431 | TTGGAAAACCCGGTGGTAT | 3173 | ATACCACCGGGTTTTCCAA |
| siRNA 1432 | 1432 | TGGAAAACCCGGTGGTATT | 3174 | AATACCACCGGGTTTTCCA |
| siRNA 1434 | 1434 | GAAAACCCGGTGGTATTCC | 3176 | GGAATACCACCGGGTTTTC |
| siRNA 1435 | 1435 | AAAACCCGGTGGTATTCCA | 3177 | TGGAATACCACCGGGTTTT |
| siRNA 1440 | 1440 | CCGGTGGTATTCCATGAAG | 3182 | CTTCATGGAATACCACCGG |
| siRNA 1461 | 1461 | AACCACTATGAAGATAATC | 3203 | GATTATCTTCATAGTGGTT |
| siRNA 1462 | 1462 | ACCACTATGAAGATAATCC | 3204 | GGATTATCTTCATAGTGGT |
| siRNA 1482 | 1482 | ATTCAACAGACTCACAATT | 3224 | AATTGTGAGTCTGTTGAAT |
| siRNA 1485 | 1485 | CAACAGACTCACAATTGGA | 3227 | TCCAATTGTGAGTCTGTTG |
| siRNA 1519 | 1519 | CACCTGGGGGGAGCCAAAC | 3261 | GTTTGGCTCCCCCCAGGTG |
| siRNA 1533 | 1533 | CAAACAGGCTGGAGACGTT | 3275 | AACGTCTCCAGCCTGTTTG |
| siRNA 1540 | 1540 | GCTGGAGACGTTTAAAAGA | 3282 | TCTTTTAAACGTCTCCAGC |
| siRNA 1588 | 1588 | GGACTTTATCTGAACAGAG | 3330 | CTCTGTTCAGATAAAGTCC |
| siRNA 1617 | 1617 | TTTTTCCTATTGGACAATG | 3359 | CATTGTCCAATAGGAAAAA |
| siRNA 1622 | 1622 | CCTATTGGACAATGGACTT | 3364 | AAGTCCATTGTCCAATAGG |
| siRNA 1623 | 1623 | CTATTGGACAATGGACTTG | 3365 | CAAGTCCATTGTCCAATAG |
| siRNA 1626 | 1626 | TTGGACAATGGACTTGCAA | 3368 | TTGCAAGTCCATTGTCCAA |
| siRNA 1627 | 1627 | TGGACAATGGACTTGCAAA | 3369 | TTTGCAAGTCCATTGTCCA |
| siRNA 1647 | 1647 | CTTCACTTCATTTTAAGAG | 3389 | CTCTTAAAATGAAGTGAAG |
| siRNA 1648 | 1648 | TTCACTTCATTTTAAGAGC | 3390 | GCTCTTAAAATGAAGTGAA |
| siRNA 1649 | 1649 | TCACTTCATTTTAAGAGCA | 3391 | TGCTCTTAAAATGAAGTGA |
| siRNA 1664 | 1664 | AGCAAAAGACCCCATGTTG | 3406 | CAACATGGGGTCTTTTGCT |
| siRNA 1672 | 1672 | ACCCCATGTTGAAAACTCC | 3414 | GGAGTTTTCAACATGGGGT |
| siRNA 1678 | 1678 | TGTTGAAAACTCCATAACA | 3420 | TGTTATGGAGTTTTCAACA |
| siRNA 1679 | 1679 | GTTGAAAACTCCATAACAG | 3421 | CTGTTATGGAGTTTTCAAC |
| siRNA 1680 | 1680 | TTGAAAACTCCATAACAGT | 3422 | ACTGTTATGGAGTTTTCAA |
| siRNA 1696 | 1696 | AGTTTTATGCTGATGATAA | 3438 | TTATCATCAGCATAAAACT |
| siRNA 1700 | 1700 | TTATGCTGATGATAATTTA | 3442 | TAAATTATCATCAGCATAA |
| siRNA 1710 | 1710 | GATAATTTATCTACATGCA | 3452 | TGCATGTAGATAAATTATC |

The siRNAs in subset C have the following characteristics:
Cross-reactivity: With 19mer in human FGG mRNA, with 17mer/19mer in NHP FGG
Specificity category: For human and NHP: AS2 or better, SS3 or better
miRNA seeds: AS+SS strand: seed region not conserved in human, mouse, and rat and not present in >4 species. AS strand: seed region not identical to seed region of known human miRNA
Off-target frequency: ≤30 human off-targets matched with 2 mismatches by antisense strand
SNPs: siRNA target sites do not harbor SNPs with a MAF≥1% (pos. 2-18)

The siRNA sequences in subset C were also selected for absence of seed regions in the AS or S strands that are identical to a seed region of known human miRNA to yield subset D. Subset D includes 147 siRNAs whose base sequences are shown in Table 6.

TABLE 6
Subset D siRNAs

| | Sense | | Antisense | |
|---|---|---|---|---|
| | strand | | strand | |
| | SEQ | Sense strand sequence | SEQ ID | Antisense strand sequence |
| siRNA Name | ID NO: | (5′-3′) | NO: | (5′-3′) |
| siRNA 233 | 233 | CAGACATCATGAGTTGGTC | 1975 | GACCAACTCATGATGTCTG |
| siRNA 251 | 251 | CCTTGCACCCCCGGAATTT | 1993 | AAATTCCGGGGGTGCAAGG |
| siRNA 254 | 254 | TGCACCCCCGGAATTTAAT | 1996 | ATTAAATTCCGGGGGTGCA |
| siRNA 255 | 255 | GCACCCCCGGAATTTAATT | 1997 | AATTAAATTCCGGGGGTGC |
| siRNA 261 | 261 | CCGGAATTTAATTCTCTAC | 2003 | GTAGAGAATTAAATTCCGG |
| siRNA 274 | 274 | CTCTACTTCTATGCTCTTT | 2016 | AAAGAGCATAGAAGTAGAG |
| siRNA 310 | 310 | TGTGTAGCATATGTTGCTA | 2052 | TAGCAACATATGCTACACA |
| siRNA 324 | 324 | TGCTACCAGAGACAACTGC | 2066 | GCAGTTGTCTCTGGTAGCA |
| siRNA 335 | 335 | ACAACTGCTGCATCTTAGA | 2077 | TCTAAGATGCAGCAGTTGT |
| siRNA 342 | 342 | CTGCATCTTAGATGAAAGA | 2084 | TCTTTCATCTAAGATGCAG |
| siRNA 343 | 343 | TGCATCTTAGATGAAAGAT | 2085 | ATCTTTCATCTAAGATGCA |
| siRNA 344 | 344 | GCATCTTAGATGAAAGATT | 2086 | AATCTTTCATCTAAGATGC |
| siRNA 347 | 347 | TCTTAGATGAAAGATTCGG | 2089 | CCGAATCTTTCATCTAAGA |
| siRNA 349 | 349 | TTAGATGAAAGATTCGGTA | 2091 | TACCGAATCTTTCATCTAA |
| siRNA 350 | 350 | TAGATGAAAGATTCGGTAG | 2092 | CTACCGAATCTTTCATCTA |
| siRNA 351 | 351 | AGATGAAAGATTCGGTAGT | 2093 | ACTACCGAATCTTTCATCT |
| siRNA 352 | 352 | GATGAAAGATTCGGTAGTT | 2094 | AACTACCGAATCTTTCATC |
| siRNA 354 | 354 | TGAAAGATTCGGTAGTTAT | 2096 | ATAACTACCGAATCTTTCA |
| siRNA 355 | 355 | GAAAGATTCGGTAGTTATT | 2097 | AATAACTACCGAATCTTTC |
| siRNA 359 | 359 | GATTCGGTAGTTATTGTCC | 2101 | GGACAATAACTACCGAATC |
| siRNA 363 | 363 | CGGTAGTTATTGTCCAACT | 2105 | AGTTGGACAATAACTACCG |
| siRNA 365 | 365 | GTAGTTATTGTCCAACTAC | 2107 | GTAGTTGGACAATAACTAC |
| siRNA 367 | 367 | AGTTATTGTCCAACTACCT | 2109 | AGGTAGTTGGACAATAACT |
| siRNA 373 | 373 | TGTCCAACTACCTGTGGCA | 2115 | TGCCACAGGTAGTTGGACA |
| siRNA 397 | 397 | GATTTCCTGTCTACTTATC | 2139 | GATAAGTAGACAGGAAATC |
| siRNA 398 | 398 | ATTTCCTGTCTACTTATCA | 2140 | TGATAAGTAGACAGGAAAT |
| siRNA 411 | 411 | TTATCAAACCAAAGTAGAC | 2153 | GTCTACTTTGGTTTGATAA |
| siRNA 423 | 423 | AGTAGACAAGGATCTACAG | 2165 | CTGTAGATCCTTGTCTACT |
| siRNA 426 | 426 | AGACAAGGATCTACAGTCT | 2168 | AGACTGTAGATCCTTGTCT |
| siRNA 432 | 432 | GGATCTACAGTCTTTGGAA | 2174 | TTCCAAAGACTGTAGATCC |
| siRNA 434 | 434 | ATCTACAGTCTTTGGAAGA | 2176 | TCTTCCAAAGACTGTAGAT |
| siRNA 447 | 447 | GGAAGACATCTTACATCAA | 2189 | TTGATGTAAGATGTCTTCC |
| siRNA 449 | 449 | AAGACATCTTACATCAAGT | 2191 | ACTTGATGTAAGATGTCTT |
| siRNA 493 | 493 | CAGCTGATAAAAGCAATCC | 2235 | GGATTGCTTTTATCAGCTG |
| siRNA 497 | 497 | TGATAAAAGCAATCCAACT | 2239 | AGTTGGATTGCTTTTATCA |
| siRNA 504 | 504 | AGCAATCCAACTCACTTAT | 2246 | ATAAGTGAGTTGGATTGCT |
| siRNA 505 | 505 | GCAATCCAACTCACTTATA | 2247 | TATAAGTGAGTTGGATTGC |
| siRNA 507 | 507 | AATCCAACTCACTTATAAT | 2249 | ATTATAAGTGAGTTGGATT |
| siRNA 508 | 508 | ATCCAACTCACTTATAATC | 2250 | GATTATAAGTGAGTTGGAT |
| siRNA 509 | 509 | TCCAACTCACTTATAATCC | 2251 | GGATTATAAGTGAGTTGGA |
| siRNA 510 | 510 | CCAACTCACTTATAATCCT | 2252 | AGGATTATAAGTGAGTTGG |
| siRNA 514 | 514 | CTCACTTATAATCCTGATG | 2256 | CATCAGGATTATAAGTGAG |
| siRNA 515 | 515 | TCACTTATAATCCTGATGA | 2257 | TCATCAGGATTATAAGTGA |
| siRNA 523 | 523 | AATCCTGATGAATCATCAA | 2265 | TTGATGATTCATCAGGATT |
| siRNA 528 | 528 | TGATGAATCATCAAAACCA | 2270 | TGGTTTTGATGATTCATCA |
| siRNA 544 | 544 | CCAAATATGATAGACGCTG | 2286 | CAGCGTCTATCATATTTGG |
| siRNA 554 | 554 | TAGACGCTGCTACTTTGAA | 2296 | TTCAAAGTAGCAGCGTCTA |
| siRNA 573 | 573 | GTCCAGGAAAATGTTAGAA | 2315 | TTCTAACATTTTCCTGGAC |
| siRNA 599 | 599 | TGAAATATGAAGCATCGAT | 2341 | ATCGATGCTTCATATTTCA |
| siRNA 600 | 600 | GAAATATGAAGCATCGATT | 2342 | AATCGATGCTTCATATTTC |
| siRNA 604 | 604 | TATGAAGCATCGATTTTAA | 2346 | TTAAAATCGATGCTTCATA |
| siRNA 606 | 606 | TGAAGCATCGATTTTAACA | 2348 | TGTTAAAATCGATGCTTCA |
| siRNA 609 | 609 | AGCATCGATTTTAACACAT | 2351 | ATGTGTTAAAATCGATGCT |
| siRNA 619 | 619 | TTAACACATGACTCAAGTA | 2361 | TACTTGAGTCATGTGTTAA |
| siRNA 624 | 624 | ACATGACTCAAGTATTCGA | 2366 | TCGAATACTTGAGTCATGT |
| siRNA 625 | 625 | CATGACTCAAGTATTCGAT | 2367 | ATCGAATACTTGAGTCATG |
| siRNA 716 | 716 | CACAGTGCCAGGAACCTTG | 2458 | CAAGGTTCCTGGCACTGTG |
| siRNA 730 | 730 | CCTTGCAAAGACACGGTGC | 2472 | GCACCGTGTCTTTGCAAGG |
| siRNA 731 | 731 | CTTGCAAAGACACGGTGCA | 2473 | TGCACCGTGTCTTTGCAAG |
| siRNA 733 | 733 | TGCAAAGACACGGTGCAAA | 2475 | TTTGCACCGTGTCTTTGCA |
| siRNA 753 | 753 | CCATGATATCACTGGGAAA | 2495 | TTTCCCAGTGATATCATGG |
| siRNA 770 | 770 | AAGATTGTCAAGACATTGC | 2512 | GCAATGTCTTGACAATCTT |
| siRNA 780 | 780 | AGACATTGCCAATAAGGGA | 2522 | TCCCTTATTGGCAATGTCT |
| siRNA 786 | 786 | TGCCAATAAGGGAGCTAAA | 2528 | TTTAGCTCCCTTATTGGCA |
| siRNA 800 | 800 | CTAAACAGAGCGGGCTTTA | 2542 | TAAAGCCCGCTCTGTTTAG |
| siRNA 802 | 802 | AAACAGAGCGGGCTTTACT | 2544 | AGTAAAGCCCGCTCTGTTT |
| siRNA 806 | 806 | AGAGCGGGCTTTACTTTAT | 2548 | ATAAAGTAAAGCCCGCTCT |
| siRNA 812 | 812 | GGCTTTACTTTATTAAACC | 2554 | GGTTTAATAAAGTAAAGCC |
| siRNA 825 | 825 | TAAACCTCTGAAAGCTAAC | 2567 | GTTAGCTTTCAGAGGTTTA |
| siRNA 833 | 833 | TGAAAGCTAACCAGCAATT | 2575 | AATTGCTGGTTAGCTTTCA |
| siRNA 850 | 850 | TTCTTAGTCTACTGTGAAA | 2592 | TTTCACAGTAGACTAAGAA |
| siRNA 894 | 894 | TGTGTTTCAGAAGAGACTT | 2636 | AAGTCTCTTCTGAAACACA |
| siRNA 904 | 904 | AAGAGACTTGATGGCAGTG | 2646 | CACTGCCATCAAGTCTCTT |
| siRNA 929 | 929 | TCAAGAAAAACTGGATTCA | 2671 | TGAATCCAGTTTTTCTTGA |
| siRNA 932 | 932 | AGAAAAACTGGATTCAATA | 2674 | TATTGAATCCAGTTTTTCT |
| siRNA 973 | 973 | TCTCCTACTGGCACAACAG | 2715 | CTGTTGTGCCAGTAGGAGA |
| siRNA 1003 | 1003 | GGAAATGAGAAGATTCATT | 2745 | AATGAATCTTCTCATTTCC |
| siRNA 1011 | 1011 | GAAGATTCATTTGATAAGC | 2753 | GCTTATCAAATGAATCTTC |
| siRNA 1013 | 1013 | AGATTCATTTGATAAGCAC | 2755 | GTGCTTATCAAATGAATCT |
| siRNA 1052 | 1052 | CATTAAGAGTGGAACTGGA | 2794 | TCCAGTTCCACTCTTAATG |
| siRNA 1060 | 1060 | GTGGAACTGGAAGACTGGA | 2802 | TCCAGTCTTCCAGTTCCAC |
| siRNA 1071 | 1071 | AGACTGGAATGGCAGAACC | 2813 | GGTTCTGCCATTCCAGTCT |
| siRNA 1085 | 1085 | GAACCAGTACTGCAGACTA | 2827 | TAGTCTGCAGTACTGGTTC |
| siRNA 1088 | 1088 | CCAGTACTGCAGACTATGC | 2830 | GCATAGTCTGCAGTACTGG |
| siRNA 1123 | 1123 | CCTGAAGCTGACAAGTACC | 2865 | GGTACTTGTCAGCTTCAGG |
| siRNA 1127 | 1127 | AAGCTGACAAGTACCGCCT | 2869 | AGGCGGTACTTGTCAGCTT |
| siRNA 1128 | 1128 | AGCTGACAAGTACCGCCTA | 2870 | TAGGCGGTACTTGTCAGCT |
| siRNA 1130 | 1130 | CTGACAAGTACCGCCTAAC | 2872 | GTTAGGCGGTACTTGTCAG |
| siRNA 1134 | 1134 | CAAGTACCGCCTAACATAT | 2876 | ATATGTTAGGCGGTACTTG |
| siRNA 1136 | 1136 | AGTACCGCCTAACATATGC | 2878 | GCATATGTTAGGCGGTACT |
| siRNA 1137 | 1137 | GTACCGCCTAACATATGCC | 2879 | GGCATATGTTAGGCGGTAC |
| siRNA 1138 | 1138 | TACCGCCTAACATATGCCT | 2880 | AGGCATATGTTAGGCGGTA |
| siRNA 1147 | 1147 | ACATATGCCTACTTCGCTG | 2889 | CAGCGAAGTAGGCATATGT |
| siRNA 1148 | 1148 | CATATGCCTACTTCGCTGG | 2890 | CCAGCGAAGTAGGCATATG |
| siRNA 1174 | 1174 | GCTGGAGATGCCTTTGATG | 2916 | CATCAAAGGCATCTCCAGC |
| siRNA 1178 | 1178 | GAGATGCCTTTGATGGCTT | 2920 | AAGCCATCAAAGGCATCTC |
| siRNA 1182 | 1182 | TGCCTTTGATGGCTTTGAT | 2924 | ATCAAAGCCATCAAAGGCA |
| siRNA 1197 | 1197 | TGATTTTGGCGATGATCCT | 2939 | AGGATCATCGCCAAAATCA |
| siRNA 1205 | 1205 | GCGATGATCCTAGTGACAA | 2947 | TTGTCACTAGGATCATCGC |
| siRNA 1215 | 1215 | TAGTGACAAGTTTTTCACA | 2957 | TGTGAAAAACTTGTCACTA |
| siRNA 1236 | 1236 | CCATAATGGCATGCAGTTC | 2978 | GAACTGCATGCCATTATGG |
| siRNA 1243 | 1243 | GGCATGCAGTTCAGTACCT | 2985 | AGGTACTGAACTGCATGCC |
| siRNA 1265 | 1265 | ACAATGACAATGATAAGTT | 3007 | AACTTATCATTGTCATTGT |
| siRNA 1278 | 1278 | TAAGTTTGAAGGCAACTGT | 3020 | ACAGTTGCCTTCAAACTTA |
| siRNA 1286 | 1286 | AAGGCAACTGTGCTGAACA | 3028 | TGTTCAGCACAGTTGCCTT |
| siRNA 1311 | 1311 | ATCTGGTTGGTGGATGAAC | 3053 | GTTCATCCACCAACCAGAT |
| siRNA 1315 | 1315 | GGTTGGTGGATGAACAAGT | 3057 | ACTTGTTCATCCACCAACC |
| siRNA 1323 | 1323 | GATGAACAAGTGTCACGCT | 3065 | AGCGTGACACTTGTTCATC |
| siRNA 1324 | 1324 | ATGAACAAGTGTCACGCTG | 3066 | CAGCGTGACACTTGTTCAT |
| siRNA 1325 | 1325 | TGAACAAGTGTCACGCTGG | 3067 | CCAGCGTGACACTTGTTCA |
| siRNA 1342 | 1342 | GGCCATCTCAATGGAGTTT | 3084 | AAACTCCATTGAGATGGCC |
| siRNA 1344 | 1344 | CCATCTCAATGGAGTTTAT | 3086 | ATAAACTCCATTGAGATGG |
| siRNA 1348 | 1348 | CTCAATGGAGTTTATTACC | 3090 | GGTAATAAACTCCATTGAG |
| siRNA 1349 | 1349 | TCAATGGAGTTTATTACCA | 3091 | TGGTAATAAACTCCATTGA |
| siRNA 1356 | 1356 | AGTTTATTACCAAGGTGGC | 3098 | GCCACCTTGGTAATAAACT |
| siRNA 1362 | 1362 | TTACCAAGGTGGCACTTAC | 3104 | GTAAGTGCCACCTTGGTAA |
| siRNA 1384 | 1384 | AAAGCATCTACTCCTAATG | 3126 | CATTAGGAGTAGATGCTTT |
| siRNA 1385 | 1385 | AAGCATCTACTCCTAATGG | 3127 | CCATTAGGAGTAGATGCTT |
| siRNA 1389 | 1389 | ATCTACTCCTAATGGTTAT | 3131 | ATAACCATTAGGAGTAGAT |
| siRNA 1390 | 1390 | TCTACTCCTAATGGTTATG | 3132 | CATAACCATTAGGAGTAGA |
| siRNA 1391 | 1391 | CTACTCCTAATGGTTATGA | 3133 | TCATAACCATTAGGAGTAG |
| siRNA 1394 | 1394 | CTCCTAATGGTTATGATAA | 3136 | TTATCATAACCATTAGGAG |
| siRNA 1396 | 1396 | CCTAATGGTTATGATAATG | 3138 | CATTATCATAACCATTAGG |
| siRNA 1401 | 1401 | TGGTTATGATAATGGCATT | 3143 | AATGCCATTATCATAACCA |
| siRNA 1404 | 1404 | TTATGATAATGGCATTATT | 3146 | AATAATGCCATTATCATAA |
| siRNA 1418 | 1418 | TTATTTGGGCCACTTGGAA | 3160 | TTCCAAGTGGCCCAAATAA |
| siRNA 1420 | 1420 | ATTTGGGCCACTTGGAAAA | 3162 | TTTTCCAAGTGGCCCAAAT |
| siRNA 1426 | 1426 | GCCACTTGGAAAACCCGGT | 3168 | ACCGGGTTTTCCAAGTGGC |
| siRNA 1462 | 1462 | ACCACTATGAAGATAATCC | 3204 | GGATTATCTTCATAGTGGT |
| siRNA 1482 | 1482 | ATTCAACAGACTCACAATT | 3224 | AATTGTGAGTCTGTTGAAT |
| siRNA 1485 | 1485 | CAACAGACTCACAATTGGA | 3227 | TCCAATTGTGAGTCTGTTG |
| siRNA 1533 | 1533 | CAAACAGGCTGGAGACGTT | 3275 | AACGTCTCCAGCCTGTTTG |
| siRNA 1540 | 1540 | GCTGGAGACGTTTAAAAGA | 3282 | TCTTTTAAACGTCTCCAGC |
| siRNA 1588 | 1588 | GGACTTTATCTGAACAGAG | 3330 | CTCTGTTCAGATAAAGTCC |
| siRNA 1617 | 1617 | TTTTTCCTATTGGACAATG | 3359 | CATTGTCCAATAGGAAAAA |
| siRNA 1622 | 1622 | CCTATTGGACAATGGACTT | 3364 | AAGTCCATTGTCCAATAGG |
| siRNA 1623 | 1623 | CTATTGGACAATGGACTTG | 3365 | CAAGTCCATTGTCCAATAG |
| siRNA 1627 | 1627 | TGGACAATGGACTTGCAAA | 3369 | TTTGCAAGTCCATTGTCCA |
| siRNA 1647 | 1647 | CTTCACTTCATTTTAAGAG | 3389 | CTCTTAAAATGAAGTGAAG |
| siRNA 1648 | 1648 | TTCACTTCATTTTAAGAGC | 3390 | GCTCTTAAAATGAAGTGAA |
| siRNA 1649 | 1649 | TCACTTCATTTTAAGAGCA | 3391 | TGCTCTTAAAATGAAGTGA |
| siRNA 1672 | 1672 | ACCCCATGTTGAAAACTCC | 3414 | GGAGTTTTCAACATGGGGT |
| siRNA 1678 | 1678 | TGTTGAAAACTCCATAACA | 3420 | TGTTATGGAGTTTTCAACA |
| siRNA 1680 | 1680 | TTGAAAACTCCATAACAGT | 3422 | ACTGTTATGGAGTTTTCAA |
| siRNA 1696 | 1696 | AGTTTTATGCTGATGATAA | 3438 | TTATCATCAGCATAAAACT |
| siRNA 1700 | 1700 | TTATGCTGATGATAATTTA | 3442 | TAAATTATCATCAGCATAA |
| siRNA 1710 | 1710 | GATAATTTATCTACATGCA | 3452 | TGCATGTAGATAAATTATC |

The siRNAs in subset D were selected to have the following characteristics:
Cross-reactivity: With 19mer in human FGG mRNA, with 17mer/19mer in NHP FGG
Specificity category: For human and NHP: AS2 or better, SS3 or better
miRNA seeds: AS+SS strand: seed region not conserved in human, mouse, and rat and not present in >4 species. AS+SS strand: seed region not identical to seed region of known human miRNA
Off-target frequency: ≤20 human off-targets matched with 2 mismatches by antisense strand
SNPs: siRNA target sites do not harbor SNPs with a MAF≥100 (pos. 2-18)

Subset E includes 53 siRNAs. The siRNAs in subset E include siRNAs from subset A and additional siRNAs that were tested in vitro (see, e.g., Table 7).

TABLE 7
Subset E siRNAs

| | Sense | | Antisense | |
|---|---|---|---|---|
| | strand | | strand | |
| | SEQ | Sense strand sequence | SEQ | Antisense strand sequence |
| siRNA Name | ID NO: | (5′-3′) | ID NO: | (5′-3′) |
| siRNA 233 | 233 | CAGACAUCAUGAGUUGGUC | 1975 | GACCAACUCAUGAUGUCUG |
| siRNA 261 | 261 | CCGGAAUUUAAUUCUCUAC | 2003 | GUAGAGAAUUAAAUUCCGG |
| siRNA 274 | 274 | CUCUACUUCUAUGCUCUUU | 2016 | AAAGAGCAUAGAAGUAGAG |
| siRNA 342 | 342 | CUGCAUCUUAGAUGAAAGA | 2084 | UCUUUCAUCUAAGAUGCAG |
| siRNA 343 | 343 | UGCAUCUUAGAUGAAAGAU | 2085 | AUCUUUCAUCUAAGAUGCA |
| siRNA 344 | 344 | GCAUCUUAGAUGAAAGAUU | 2086 | AAUCUUUCAUCUAAGAUGC |
| siRNA 347 | 347 | UCUUAGAUGAAAGAUUCGG | 2089 | CCGAAUCUUUCAUCUAAGA |
| siRNA 349 | 349 | UUAGAUGAAAGAUUCGGUA | 2091 | UACCGAAUCUUUCAUCUAA |
| siRNA 350 | 350 | UAGAUGAAAGAUUCGGUAG | 2092 | CUACCGAAUCUUUCAUCUA |
| siRNA 351 | 351 | AGAUGAAAGAUUCGGUAGU | 2093 | ACUACCGAAUCUUUCAUCU |
| siRNA 352 | 352 | GAUGAAAGAUUCGGUAGUU | 2094 | AACUACCGAAUCUUUCAUC |
| siRNA 354 | 354 | UGAAAGAUUCGGUAGUUAU | 2096 | AUAACUACCGAAUCUUUCA |
| siRNA 355 | 355 | GAAAGAUUCGGUAGUUAUU | 2097 | AAUAACUACCGAAUCUUUC |
| siRNA 359 | 359 | GAUUCGGUAGUUAUUGUCC | 2101 | GGACAAUAACUACCGAAUC |
| siRNA 363 | 363 | CGGUAGUUAUUGUCCAACU | 2105 | AGUUGGACAAUAACUACCG |
| siRNA 365 | 365 | GUAGUUAUUGUCCAACUAC | 2107 | GUAGUUGGACAAUAACUAC |
| siRNA 367 | 367 | AGUUAUUGUCCAACUACCU | 2109 | AGGUAGUUGGACAAUAACU |
| siRNA 397 | 397 | GAUUUCCUGUCUACUUAUC | 2139 | GAUAAGUAGACAGGAAAUC |
| siRNA 398 | 398 | AUUUCCUGUCUACUUAUCA | 2140 | UGAUAAGUAGACAGGAAAU |
| siRNA 423 | 423 | AGUAGACAAGGAUCUACAG | 2165 | CUGUAGAUCCUUGUCUACU |
| siRNA 432 | 432 | GGAUCUACAGUCUUUGGAA | 2174 | UUCCAAAGACUGUAGAUCC |
| siRNA 434 | 434 | AUCUACAGUCUUUGGAAGA | 2176 | UCUUCCAAAGACUGUAGAU |
| siRNA 447 | 447 | GGAAGACAUCUUACAUCAA | 2189 | UUGAUGUAAGAUGUCUUCC |
| siRNA 449 | 449 | AAGACAUCUUACAUCAAGU | 2191 | ACUUGAUGUAAGAUGUCUU |
| siRNA 493 | 493 | CAGCUGAUAAAAGCAAUCC | 2235 | GGAUUGCUUUUAUCAGCUG |
| siRNA 497 | 497 | UGAUAAAAGCAAUCCAACU | 2239 | AGUUGGAUUGCUUUUAUCA |
| siRNA 523 | 523 | AAUCCUGAUGAAUCAUCAA | 2265 | UUGAUGAUUCAUCAGGAUU |
| siRNA 528 | 528 | UGAUGAAUCAUCAAAACCA | 2270 | UGGUUUUGAUGAUUCAUCA |
| siRNA 573 | 573 | GUCCAGGAAAAUGUUAGAA | 2315 | UUCUAACAUUUUCCUGGAC |
| siRNA 753 | 753 | CCAUGAUAUCACUGGGAAA | 2495 | UUUCCCAGUGAUAUCAUGG |
| siRNA 770 | 770 | AAGAUUGUCAAGACAUUGC | 2512 | GCAAUGUCUUGACAAUCUU |
| siRNA 806 | 806 | AGAGCGGGCUUUACUUUAU | 2548 | AUAAAGUAAAGCCCGCUCU |
| siRNA 825 | 825 | UAAACCUCUGAAAGCUAAC | 2567 | GUUAGCUUUCAGAGGUUUA |
| siRNA 929 | 929 | UCAAGAAAAACUGGAUUCA | 2671 | UGAAUCCAGUUUUUCUUGA |
| siRNA 932 | 932 | AGAAAAACUGGAUUCAAUA | 2674 | UAUUGAAUCCAGUUUUUCU |
| siRNA 1003 | 1003 | GGAAAUGAGAAGAUUCAUU | 2745 | AAUGAAUCUUCUCAUUUCC |
| siRNA 1011 | 1011 | GAAGAUUCAUUUGAUAAGC | 2753 | GCUUAUCAAAUGAAUCUUC |
| siRNA 1013 | 1013 | AGAUUCAUUUGAUAAGCAC | 2755 | GUGCUUAUCAAAUGAAUCU |
| siRNA 1052 | 1052 | CAUUAAGAGUGGAACUGGA | 2794 | UCCAGUUCCACUCUUAAUG |
| siRNA 1265 | 1265 | ACAAUGACAAUGAUAAGUU | 3007 | AACUUAUCAUUGUCAUUGU |
| siRNA 1278 | 1278 | UAAGUUUGAAGGCAACUGU | 3020 | ACAGUUGCCUUCAAACUUA |
| siRNA 1348 | 1348 | CUCAAUGGAGUUUAUUACC | 3090 | GGUAAUAAACUCCAUUGAG |
| siRNA 1349 | 1349 | UCAAUGGAGUUUAUUACCA | 3091 | UGGUAAUAAACUCCAUUGA |
| siRNA 1356 | 1356 | AGUUUAUUACCAAGGUGGC | 3098 | GCCACCUUGGUAAUAAACU |
| siRNA 1384 | 1384 | AAAGCAUCUACUCCUAAUG | 3126 | CAUUAGGAGUAGAUGCUUU |
| siRNA 1385 | 1385 | AAGCAUCUACUCCUAAUGG | 3127 | CCAUUAGGAGUAGAUGCUU |
| siRNA 1389 | 1389 | AUCUACUCCUAAUGGUUAU | 3131 | AUAACCAUUAGGAGUAGAU |
| siRNA 1390 | 1390 | UCUACUCCUAAUGGUUAUG | 3132 | CAUAACCAUUAGGAGUAGA |
| siRNA 1391 | 1391 | CUACUCCUAAUGGUUAUGA | 3133 | UCAUAACCAUUAGGAGUAG |
| siRNA 1394 | 1394 | CUCCUAAUGGUUAUGAUAA | 3136 | UUAUCAUAACCAUUAGGAG |
| siRNA 1396 | 1396 | CCUAAUGGUUAUGAUAAUG | 3138 | CAUUAUCAUAACCAUUAGG |
| siRNA 1418 | 1418 | UUAUUUGGGCCACUUGGAA | 3160 | UUCCAAGUGGCCCAAAUAA |
| siRNA 1462 | 1462 | ACCACUAUGAAGAUAAUCC | 3204 | GGAUUAUCUUCAUAGUGGU |

In some cases, the sense strand of any of the siRNAs of subset E comprises siRNA with a particular modification pattern. In this example modification pattern, position 9 counting from the 5′ end of the of the sense strand is has the 2′F modification. Where a “2′F modification” is denoted, it is intended to mean that a 2′F is included. In this example modification pattern, when position 9 of the sense strand is a pyrimidine, then all purines in the sense strand have the 2′Ome modification. Where a “2′Ome modification” is denoted, it is intended to mean that a 2′Ome is included. In this example modification pattern, when position 9 is the only pyrimidine between positions 5 and 11 of the sense stand, then position 9 is the only position with the 2′F modification in the sense strand. In this example modification pattern, when position 9 and only one other base between positions 5 and 11 of the sense strand are pyrimidines, then both of these pyrimidines are the only two positions with the 2′F modification in the sense strand. In this example modification pattern, when position 9 and only two other bases between positions 5 and 11 of the sense strand are pyrimidines, and those two other pyrimidines 're in adjacent positions so that there would be not three 2′F modifications in a row, then any combination of 2′F modifications can be made that give three 2′F modifications in total. In this example modification pattern, when there are >2 pyrimidines between positions 5 and 11 of the sense strand, then all combinations of pyrimidines having the 2′F modification are allowed that have three to five 2′F modifications in total, provided that the sense strand does not have three 2′F modifications in a row.

In this example modification pattern, when position 9 of the sense strand is a purine, then all purines in the sense strand have the 2′Ome modification. In this example modification pattern, when position 9 is the only purine between positions 5 and 11 of the sense stand, then position 9 is the only position with the 2′F modification in the sense strand. In this example modification pattern, when position 9 and only one other base between positions 5 and 11 of the sense strand are purines, then both of these purines are the only two positions with the 2′F modification in the sense strand. In this example modification pattern, when position 9 and only two other bases between positions 5 and 11 of the sense strand are purines, and those two other purines are in adjacent positions so that there would be not three 2′F modifications in a row, then any combination of 2′F modifications can be made that give three 2′F modifications in total. In this example modification pattern, when there are >2 purines between positions 5 and 11 of the sense strand, then all combinations of purines having the 2′F modification are allowed that have three to five 2′F modifications in total, provided that the sense strand does not have three 2′F modifications in a row. In some cases, the sense strand of any of the siRNAs of subset E comprises a modification pattern which conforms to these sense strand rules (Table 8A).

In some cases, the antisense strand of any of the siRNAs of subset E comprises modification pattern 9AS (Table 8A). The siRNAs in subset E may comprise any other modification pattern(s).

TABLE 8A
Modified siRNA sequences

| | Sense | | Antisense | |
|---|---|---|---|---|
| | strand | | strand | |
| siRNA | SEQ ID | Sense strand sequence | SEQ | Antisense strand sequence |
| Name | NO: | (5′-3′) | ID NO: | (5′-3′) |
| ETD01663 | 3485 | cagacAfucAfuGfaguugguasusu | 3538 | uAfcCfaAfcUfcAfuGfaUfgUfcUfgsusu |
| ETD01664 | 3486 | ccggaaUfuUfaauucucuaasusu | 3539 | uUfaGfaGfaAfuUfaAfaUfuCfcGfgsusu |
| ETD01665 | 3487 | cucuaCfUfUfCfUfaugcucuuasusu | 3540 | uAfaGfaGfcAfuAfgAfaGfuAfgAfgsusu |
| ETD01666 | 3488 | cugcaUfCfUfUfagaugaaagasusu | 3541 | uCfuUfuCfaUfcUfaAfgAfuGfcAfgsusu |
| ETD01667 | 3489 | ugcaucuuAfGfaugaaagaasusu | 3542 | uUfcUfuUfcAfuCfuAfaGfaUfgCfasusu |
| ETD01668 | 3490 | gcaucuuAfGfaugaaagauasusu | 3543 | uAfuCfuUfuCfaUfcUfaAfgAfuGfcsusu |
| ETD01669 | 3491 | ucuuAfGfauGfaAfagauucgasusu | 3544 | uCfgAfaUfcUfuUfcAfuCfuAfaGfasusu |
| ETD01670 | 3492 | uuagAfuGfAfAfAfgauucgguasusu | 3545 | uAfcCfgAfaUfcUfuUfcAfuCfuAfasusu |
| ETD01671 | 3493 | uagauGfAfAfAfgAfuucgguaasusu | 3546 | uUfaCfcGfaAfuCfuUfuCfaUfcUfasusu |
| ETD01672 | 3494 | agauGfAfAfAfGfauucgguagasusu | 3547 | uCfuAfcCfgAfaUfcUfuUfcAfuCfususu |
| ETD01673 | 3495 | gaugAfAfAfGfAfuucgguaguasusu | 3548 | uAfcUfaCfcGfaAfuCfuUfuCfaUfcsusu |
| ETD01674 | 3496 | ugaaagaUfUfcgguaguuaasusu | 3549 | uUfaAfcUfaCfcGfaAfuCfuUfuCfasusu |
| ETD01675 | 3497 | gaaagaUfuCfgguaguuauasusu | 3550 | uAfuAfaCfuAfcCfgAfaUfcUfuUfcsusu |
| ETD01676 | 3498 | gauucGfGfuAfGfuuauugucasusu | 3551 | uGfaCfaAfuAfaCfuAfcCfgAfaUfcsusu |
| ETD01677 | 3499 | cgguAfGfuuAfuuguccaacasusu | 3552 | uGfuUfgGfaCfaAfuAfaCfuAfcCfgsusu |
| ETD01678 | 3500 | guagUfUfaUfUfgUfccaacuaasusu | 3553 | uUfaGfuUfgGfaCfaAfuAfaCfuAfcsusu |
| ETD01679 | 3501 | aguuaUfUfgUfcCfaacuaccasusu | 3554 | uGfgUfaGfuUfgGfaCfaAfuAfaCfususu |
| ETD01680 | 3502 | gauuuccuGfucuacuuauasusu | 3555 | uAfuAfaGfuAfgAfcAfgGfaAfaUfcsusu |
| ETD01681 | 3503 | auuuCfCfugUfcUfacuuaucasusu | 3556 | uGfaUfaAfgUfaGfaCfaGfgAfaAfususu |
| ETD01682 | 3504 | aguaGfAfcAfAfgGfaucuacaasusu | 3557 | uUfgUfaGfaUfcCfuUfgUfcUfaCfususu |
| ETD01683 | 3505 | ggaucuAfcAfGfucuuuggaasusu | 3558 | uUfcCfaAfaGfaCfuGfuAfgAfuCfcsusu |
| ETD01684 | 3506 | aucuaCfagUfcUfuuggaagasusu | 3559 | uCfuUfcCfaAfaGfaCfuGfuAfgAfususu |
| ETD01685 | 3507 | ggaagaCfaUfcUfuacaucaasusu | 3560 | uUfgAfuGfuAfaGfaUfgUfcUfuCfcsusu |
| ETD01686 | 3508 | aagaCfaUfCfUfUfacaucaagasusu | 3561 | uCfuUfgAfuGfuAfaGfaUfgUfcUfususu |
| ETD01687 | 3509 | cagcuGfAfuAfaAfagcaaucasusu | 3562 | uGfaUfuGfcUfuUfuAfuCfaGfcUfgsusu |
| ETD01688 | 3510 | ugauAfAfaAfGfcAfauccaacasusu | 3563 | uGfuUfgGfaUfuGfcUfuUfuAfuCfasusu |
| ETD01689 | 3511 | aaucCfUfgaUfgaaucaucaasusu | 3564 | uUfgAfuGfaUfuCfaUfcAfgGfaUfususu |
| ETD01690 | 3512 | ugaugaaUfCfaUfcaaaaccasusu | 3565 | uGfgUfuUfuGfaUfgAfuUfcAfuCfasusu |
| ETD01691 | 3513 | guccAfGfgAfAfaAfuguuagaasusu | 3566 | uUfcUfaAfcAfuUfuUfcCfuGfgAfcsusu |
| ETD01692 | 3514 | ccaugaUfaUfCfacugggaaasusu | 3567 | uUfuCfcCfaGfuGfaUfaUfcAfuGfgsusu |
| ETD01693 | 3515 | aagaUfUfgUfCfaagacauugasusu | 3568 | uCfaAfuGfuCfuUfgAfcAfaUfcUfususu |
| ETD01694 | 3516 | agagCfgggCfuUfuacuuuaasusu | 3569 | uUfaAfaGfuAfaAfgCfcCfgCfuCfususu |
| ETD01695 | 3517 | uaaaCfCfUfCfUfgaaagcuaaasusu | 3570 | uUfuAfgCfuUfuCfaGfaGfgUfuUfasusu |
| ETD01696 | 3518 | ucaaGfAfAfAfAfacuggauucasusu | 3571 | uGfaAfuCfcAfgUfuUfuUfcUfuGfasusu |
| ETD01697 | 3519 | agaaaaaCfUfggauucaauasusu | 3572 | uAfuUfgAfaUfcCfaGfuUfuUfuCfususu |
| ETD01698 | 3520 | ggaaAfuGfAfGfAfagauucauasusu | 3573 | uAfuGfaAfuCfuUfcUfcAfuUfuCfcsusu |
| ETD01699 | 3521 | gaagAfuucAfuuugauaagasusu | 3574 | uCfuUfaUfcAfaAfuGfaAfuCfuUfcsusu |
| ETD01700 | 3522 | agauUfCfaUfUfugauaagcaasusu | 3575 | uUfgCfuUfaUfcAfaAfuGfaAfuCfususu |
| ETD01701 | 3523 | cauuAfAfgAfGfuGfgaacuggasusu | 3576 | uCfcAfgUfuCfcAfcUfcUfuAfaUfgsusu |
| ETD01702 | 3524 | acaauGfAfcAfAfugauaaguasusu | 3577 | uAfcUfuAfuCfaUfuGfuCfaUfuGfususu |
| ETD01703 | 3525 | uaaguuuGfAfAfGfgcaacugasusu | 3578 | uCfaGfuUfgCfcUfuCfaAfaCfuUfasusu |
| ETD01704 | 3526 | cucaAfuGfGfAfGfuuuauuacasusu | 3579 | uGfuAfaUfaAfaCfuCfcAfuUfgAfgsusu |
| ETD01705 | 3527 | ucaauGfGfAfGfuuuauuaccasusu | 3580 | uGfgUfaAfuAfaAfcUfcCfaUfuGfasusu |
| ETD01706 | 3528 | aguuuAfuuAfccaagguggasusu | 3581 | uCfcAfcCfuUfgGfuAfaUfaAfaCfususu |
| ETD01707 | 3529 | aaagCfaUfcUfaCfuccuaauasusu | 3582 | uAfuUfaGfgAfgUfaGfaUfgCfuUfususu |
| ETD01708 | 3530 | aagcAfucuAfcuccuaaugasusu | 3583 | uCfaUfuAfgGfaGfuAfgAfuGfcUfususu |
| ETD01709 | 3531 | aucuaCfUfCfCfUfaaugguuaasusu | 3584 | uUfaAfcCfaUfuAfgGfaGfuAfgAfususu |
| ETD01710 | 3532 | ucuaCfUfCfCfUfaaugguuauasusu | 3585 | uAfuAfaCfcAfuUfaGfgAfgUfaGfasusu |
| ETD01711 | 3533 | cuacuccuAfAfugguuaugasusu | 3586 | uCfaUfaAfcCfaUfuAfgGfaGfuAfgsusu |
| ETD01712 | 3534 | cuccuAfAfuGfGfuuaugauaasusu | 3587 | uUfaUfcAfuAfaCfcAfuUfaGfgAfgsusu |
| ETD01713 | 3535 | ccuaaUfggUfUfaugauaauasusu | 3588 | uAfuUfaUfcAfuAfaCfcAfuUfaGfgsusu |
| ETD01714 | 3536 | uuauuuGfgGfccacuuggaasusu | 3589 | uUfcCfaAfgUfgGfcCfcAfaAfuAfasusu |
| ETD01715 | 3537 | accacuAfuGfaAfgauaaucasusu | 3590 | uGfaUfuAfuCfuUfcAfuAfgUfgGfususu |

In Table 8A, Nf (Af, Cf, Gf, Uf, or Tf) is a 2′ fluoro-modified nucleoside, a (a, c, g, u, or t) is a 2′ O-methyl modified nucleoside. and “s” is a phosphorothioate linkage.

Any siRNA among any of subsets A-E may comprise any modification pattern described herein. If a sequence is a different number of nucleotides in length than a modification pattern, the modification pattern may still be used with the appropriate number of additional nucleotides added 5′ or 3′ to match the number of nucleotides in the modification pattern. For example, if a sense or antisense strand of the siRNA among any of subsets A-E comprises 19 nucleotides, and a modification pattern comprises 21 nucleotides, UU may be added onto the 5′ end of the sense or antisense strand. Using a different algorithm for analyzing siRNA specificity, an additional bioinformatically selected set of siRNAs was generated. Prioritizing sequences for target specificity, species cross-reactivity, miRNA seed region sequences and SNPs as described above yields subset G. Subset G contains 131 siRNAs whose base sequences are shown in Table 8B.

TABLE 8B
Subset G siRNAs

| Sense | | Antisense | |
|---|---|---|---|
| strand | | strand | |
| SEQ | Sense strand sequence | SEQ ID | Antisense strand sequence |
| ID NO: | (5′-3′) | NO: | (5′-3′) |
| 236 | ACATCATGAGTTGGTCCTT | 1978 | AAGGACCAACTCATGATGT |
| 243 | GAGTTGGTCCTTGCACCCC | 1985 | GGGGTGCAAGGACCAACTC |
| 259 | CCCCGGAATTTAATTCTCT | 2001 | AGAGAATTAAATTCCGGGG |
| 264 | GAATTTAATTCTCTACTTC | 2006 | GAAGTAGAGAATTAAATTC |
| 276 | CTACTTCTATGCTCTTTTA | 2018 | TAAAAGAGCATAGAAGTAG |
| 305 | CAACATGTGTAGCATATGT | 2047 | ACATATGCTACACATGTTG |
| 307 | ACATGTGTAGCATATGTTG | 2049 | CAACATATGCTACACATGT |
| 312 | TGTAGCATATGTTGCTACC | 2054 | GGTAGCAACATATGCTACA |
| 321 | TGTTGCTACCAGAGACAAC | 2063 | GTTGTCTCTGGTAGCAACA |
| 325 | GCTACCAGAGACAACTGCT | 2067 | AGCAGTTGTCTCTGGTAGC |
| 326 | CTACCAGAGACAACTGCTG | 2068 | CAGCAGTTGTCTCTGGTAG |
| 330 | CAGAGACAACTGCTGCATC | 2072 | GATGCAGCAGTTGTCTCTG |
| 346 | ATCTTAGATGAAAGATTCG | 2088 | CGAATCTTTCATCTAAGAT |
| 357 | AAGATTCGGTAGTTATTGT | 2099 | ACAATAACTACCGAATCTT |
| 370 | TATTGTCCAACTACCTGTG | 2112 | CACAGGTAGTTGGACAATA |
| 371 | ATTGTCCAACTACCTGTGG | 2113 | CCACAGGTAGTTGGACAAT |
| 374 | GTCCAACTACCTGTGGCAT | 2116 | ATGCCACAGGTAGTTGGAC |
| 375 | TCCAACTACCTGTGGCATT | 2117 | AATGCCACAGGTAGTTGGA |
| 385 | TGTGGCATTGCAGATTTCC | 2127 | GGAAATCTGCAATGCCACA |
| 393 | TGCAGATTTCCTGTCTACT | 2135 | AGTAGACAGGAAATCTGCA |
| 408 | TACTTATCAAACCAAAGTA | 2150 | TACTTTGGTTTGATAAGTA |
| 413 | ATCAAACCAAAGTAGACAA | 2155 | TTGTCTACTTTGGTTTGAT |
| 425 | TAGACAAGGATCTACAGTC | 2167 | GACTGTAGATCCTTGTCTA |
| 443 | CTTTGGAAGACATCTTACA | 2185 | TGTAAGATGTCTTCCAAAG |
| 451 | GACATCTTACATCAAGTTG | 2193 | CAACTTGATGTAAGATGTC |
| 496 | CTGATAAAAGCAATCCAAC | 2238 | GTTGGATTGCTTTTATCAG |
| 498 | GATAAAAGCAATCCAACTC | 2240 | GAGTTGGATTGCTTTTATC |
| 503 | AAGCAATCCAACTCACTTA | 2245 | TAAGTGAGTTGGATTGCTT |
| 506 | CAATCCAACTCACTTATAA | 2248 | TTATAAGTGAGTTGGATTG |
| 545 | CAAATATGATAGACGCTGC | 2287 | GCAGCGTCTATCATATTTG |
| 553 | ATAGACGCTGCTACTTTGA | 2295 | TCAAAGTAGCAGCGTCTAT |
| 560 | CTGCTACTTTGAAGTCCAG | 2302 | CTGGACTTCAAAGTAGCAG |
| 565 | ACTTTGAAGTCCAGGAAAA | 2307 | TTTTCCTGGACTTCAAAGT |
| 602 | AATATGAAGCATCGATTTT | 2344 | AAAATCGATGCTTCATATT |
| 603 | ATATGAAGCATCGATTTTA | 2345 | TAAAATCGATGCTTCATAT |
| 626 | ATGACTCAAGTATTCGATA | 2368 | TATCGAATACTTGAGTCAT |
| 670 | AATCAAAAGATTGTTAACC | 2412 | GGTTAACAATCTTTTGATT |
| 674 | AAAAGATTGTTAACCTGAA | 2416 | TTCAGGTTAACAATCTTTT |
| 694 | GAGAAGGTAGCCCAGCTTG | 2436 | CAAGCTGGGCTACCTTCTC |
| 726 | GGAACCTTGCAAAGACACG | 2468 | CGTGTCTTTGCAAGGTTCC |
| 742 | ACGGTGCAAATCCATGATA | 2484 | TATCATGGATTTGCACCGT |
| 743 | CGGTGCAAATCCATGATAT | 2485 | ATATCATGGATTTGCACCG |
| 747 | GCAAATCCATGATATCACT | 2489 | AGTGATATCATGGATTTGC |
| 755 | ATGATATCACTGGGAAAGA | 2497 | TCTTTCCCAGTGATATCAT |
| 776 | GTCAAGACATTGCCAATAA | 2518 | TTATTGGCAATGTCTTGAC |
| 779 | AAGACATTGCCAATAAGGG | 2521 | CCCTTATTGGCAATGTCTT |
| 789 | CAATAAGGGAGCTAAACAG | 2531 | CTGTTTAGCTCCCTTATTG |
| 793 | AAGGGAGCTAAACAGAGCG | 2535 | CGCTCTGTTTAGCTCCCTT |
| 799 | GCTAAACAGAGCGGGCTTT | 2541 | AAAGCCCGCTCTGTTTAGC |
| 803 | AACAGAGCGGGCTTTACTT | 2545 | AAGTAAAGCCCGCTCTGTT |
| 836 | AAGCTAACCAGCAATTCTT | 2578 | AAGAATTGCTGGTTAGCTT |
| 856 | GTCTACTGTGAAATCGATG | 2598 | CATCGATTTCACAGTAGAC |
| 867 | AATCGATGGGTCTGGAAAT | 2609 | ATTTCCAGACCCATCGATT |
| 875 | GGTCTGGAAATGGATGGAC | 2617 | GTCCATCCATTTCCAGACC |
| 876 | GTCTGGAAATGGATGGACT | 2618 | AGTCCATCCATTTCCAGAC |
| 950 | ATAAAGAAGGATTTGGACA | 2692 | TGTCCAAATCCTTCTTTAT |
| 970 | CTGTCTCCTACTGGCACAA | 2712 | TTGTGCCAGTAGGAGACAG |
| 971 | TGTCTCCTACTGGCACAAC | 2713 | GTTGTGCCAGTAGGAGACA |
| 985 | ACAACAGAATTTTGGCTGG | 2727 | CCAGCCAAAATTCTGTTGT |
| 1000 | CTGGGAAATGAGAAGATTC | 2742 | GAATCTTCTCATTTCCCAG |
| 1008 | TGAGAAGATTCATTTGATA | 2750 | TATCAAATGAATCTTCTCA |
| 1009 | GAGAAGATTCATTTGATAA | 2751 | TTATCAAATGAATCTTCTC |
| 1020 | TTTGATAAGCACACAGTCT | 2762 | AGACTGTGTGCTTATCAAA |
| 1023 | GATAAGCACACAGTCTGCC | 2765 | GGCAGACTGTGTGCTTATC |
| 1033 | CAGTCTGCCATCCCATATG | 2775 | CATATGGGATGGCAGACTG |
| 1037 | CTGCCATCCCATATGCATT | 2779 | AATGCATATGGGATGGCAG |
| 1038 | TGCCATCCCATATGCATTA | 2780 | TAATGCATATGGGATGGCA |
| 1044 | CCCATATGCATTAAGAGTG | 2786 | CACTCTTAATGCATATGGG |
| 1045 | CCATATGCATTAAGAGTGG | 2787 | CCACTCTTAATGCATATGG |
| 1081 | GGCAGAACCAGTACTGCAG | 2823 | CTGCAGTACTGGTTCTGCC |
| 1090 | AGTACTGCAGACTATGCCA | 2832 | TGGCATAGTCTGCAGTACT |
| 1099 | GACTATGCCATGTTCAAGG | 2841 | CCTTGAACATGGCATAGTC |
| 1100 | ACTATGCCATGTTCAAGGT | 2842 | ACCTTGAACATGGCATAGT |
| 1114 | AAGGTGGGACCTGAAGCTG | 2856 | CAGCTTCAGGTCCCACCTT |
| 1152 | TGCCTACTTCGCTGGTGGG | 2894 | CCCACCAGCGAAGTAGGCA |
| 1156 | TACTTCGCTGGTGGGGATG | 2898 | CATCCCCACCAGCGAAGTA |
| 1196 | TTGATTTTGGCGATGATCC | 2938 | GGATCATCGCCAAAATCAA |
| 1199 | ATTTTGGCGATGATCCTAG | 2941 | CTAGGATCATCGCCAAAAT |
| 1211 | ATCCTAGTGACAAGTTTTT | 2953 | AAAAACTTGTCACTAGGAT |
| 1212 | TCCTAGTGACAAGTTTTTC | 2954 | GAAAAACTTGTCACTAGGA |
| 1213 | CCTAGTGACAAGTTTTTCA | 2955 | TGAAAAACTTGTCACTAGG |
| 1218 | TGACAAGTTTTTCACATCC | 2960 | GGATGTGAAAAACTTGTCA |
| 1219 | GACAAGTTTTTCACATCCC | 2961 | GGGATGTGAAAAACTTGTC |
| 1231 | ACATCCCATAATGGCATGC | 2973 | GCATGCCATTATGGGATGT |
| 1232 | CATCCCATAATGGCATGCA | 2974 | TGCATGCCATTATGGGATG |
| 1241 | ATGGCATGCAGTTCAGTAC | 2983 | GTACTGAACTGCATGCCAT |
| 1245 | CATGCAGTTCAGTACCTGG | 2987 | CCAGGTACTGAACTGCATG |
| 1250 | AGTTCAGTACCTGGGACAA | 2992 | TTGTCCCAGGTACTGAACT |
| 1252 | TTCAGTACCTGGGACAATG | 2994 | CATTGTCCCAGGTACTGAA |
| 1269 | TGACAATGATAAGTTTGAA | 3011 | TTCAAACTTATCATTGTCA |
| 1273 | AATGATAAGTTTGAAGGCA | 3015 | TGCCTTCAAACTTATCATT |
| 1279 | AAGTTTGAAGGCAACTGTG | 3021 | CACAGTTGCCTTCAAACTT |
| 1303 | CAGGATGGATCTGGTTGGT | 3045 | ACCAACCAGATCCATCCTG |
| 1305 | GGATGGATCTGGTTGGTGG | 3047 | CCACCAACCAGATCCATCC |
| 1327 | AACAAGTGTCACGCTGGCC | 3069 | GGCCAGCGTGACACTTGTT |
| 1329 | CAAGTGTCACGCTGGCCAT | 3071 | ATGGCCAGCGTGACACTTG |
| 1334 | GTCACGCTGGCCATCTCAA | 3076 | TTGAGATGGCCAGCGTGAC |
| 1355 | GAGTTTATTACCAAGGTGG | 3097 | CCACCTTGGTAATAAACTC |
| 1357 | GTTTATTACCAAGGTGGCA | 3099 | TGCCACCTTGGTAATAAAC |
| 1363 | TACCAAGGTGGCACTTACT | 3105 | AGTAAGTGCCACCTTGGTA |
| 1364 | ACCAAGGTGGCACTTACTC | 3106 | GAGTAAGTGCCACCTTGGT |
| 1367 | AAGGTGGCACTTACTCAAA | 3109 | TTTGAGTAAGTGCCACCTT |
| 1371 | TGGCACTTACTCAAAAGCA | 3113 | TGCTTTTGAGTAAGTGCCA |
| 1388 | CATCTACTCCTAATGGTTA | 3130 | TAACCATTAGGAGTAGATG |
| 1392 | TACTCCTAATGGTTATGAT | 3134 | ATCATAACCATTAGGAGTA |
| 1424 | GGGCCACTTGGAAAACCCG | 3166 | CGGGTTTTCCAAGTGGCCC |
| 1429 | ACTTGGAAAACCCGGTGGT | 3171 | ACCACCGGGTTTTCCAAGT |
| 1430 | CTTGGAAAACCCGGTGGTA | 3172 | TACCACCGGGTTTTCCAAG |
| 1433 | GGAAAACCCGGTGGTATTC | 3175 | GAATACCACCGGGTTTTCC |
| 1471 | AAGATAATCCCATTCAACA | 3213 | TGTTGAATGGGATTATCTT |
| 1472 | AGATAATCCCATTCAACAG | 3214 | CTGTTGAATGGGATTATCT |
| 1476 | AATCCCATTCAACAGACTC | 3218 | GAGTCTGTTGAATGGGATT |
| 1486 | AACAGACTCACAATTGGAG | 3228 | CTCCAATTGTGAGTCTGTT |
| 1489 | AGACTCACAATTGGAGAAG | 3231 | CTTCTCCAATTGTGAGTCT |
| 1516 | CACCACCTGGGGGGAGCCA | 3258 | TGGCTCCCCCCAGGTGGTG |
| 1518 | CCACCTGGGGGGAGCCAAA | 3260 | TTTGGCTCCCCCCAGGTGG |
| 1524 | GGGGGGAGCCAAACAGGCT | 3266 | AGCCTGTTTGGCTCCCCCC |
| 1525 | GGGGGAGCCAAACAGGCTG | 3267 | CAGCCTGTTTGGCTCCCCC |
| 1526 | GGGGAGCCAAACAGGCTGG | 3268 | CCAGCCTGTTTGGCTCCCC |
| 1534 | AAACAGGCTGGAGACGTTT | 3276 | AAACGTCTCCAGCCTGTTT |
| 1539 | GGCTGGAGACGTTTAAAAG | 3281 | CTTTTAAACGTCTCCAGCC |
| 1593 | TTATCTGAACAGAGAGATA | 3335 | TATCTCTCTGTTCAGATAA |
| 1594 | TATCTGAACAGAGAGATAT | 3336 | ATATCTCTCTGTTCAGATA |
| 1598 | TGAACAGAGAGATATAATA | 3340 | TATTATATCTCTCTGTTCA |
| 1600 | AACAGAGAGATATAATATT | 3342 | AATATTATATCTCTCTGTT |
| 1601 | ACAGAGAGATATAATATTT | 3343 | AAATATTATATCTCTCTGT |
| 1614 | ATATTTTTCCTATTGGACA | 3356 | TGTCCAATAGGAAAAATAT |
| 1616 | ATTTTTCCTATTGGACAAT | 3358 | ATTGTCCAATAGGAAAAAT |
| 1650 | CACTTCATTTTAAGAGCAA | 3392 | TTGCTCTTAAAATGAAGTG |
| 1693 | AACAGTTTTATGCTGATGA | 3435 | TCATCAGCATAAAACTGTT |
| 1703 | TGCTGATGATAATTTATCT | 3445 | AGATAAATTATCATCAGCA |
| 1044 | CCCATATGCATTAAGAGTG | 2786 | CACTCTTAATGCATATGGG |
| 1045 | CCATATGCATTAAGAGTGG | 2787 | CCACTCTTAATGCATATGG |
| 1081 | GGCAGAACCAGTACTGCAG | 2823 | CTGCAGTACTGGTTCTGCC |
| 1090 | AGTACTGCAGACTATGCCA | 2832 | TGGCATAGTCTGCAGTACT |
| 1099 | GACTATGCCATGTTCAAGG | 2841 | CCTTGAACATGGCATAGTC |
| 1100 | ACTATGCCATGTTCAAGGT | 2842 | ACCTTGAACATGGCATAGT |
| 1114 | AAGGTGGGACCTGAAGCTG | 2856 | CAGCTTCAGGTCCCACCTT |
| 1152 | TGCCTACTTCGCTGGTGGG | 2894 | CCCACCAGCGAAGTAGGCA |
| 1156 | TACTTCGCTGGTGGGGATG | 2898 | CATCCCCACCAGCGAAGTA |
| 1196 | TTGATTTTGGCGATGATCC | 2938 | GGATCATCGCCAAAATCAA |
| 1199 | ATTTTGGCGATGATCCTAG | 2941 | CTAGGATCATCGCCAAAAT |
| 1211 | ATCCTAGTGACAAGTTTTT | 2953 | AAAAACTTGTCACTAGGAT |
| 1212 | TCCTAGTGACAAGTTTTTC | 2954 | GAAAAACTTGTCACTAGGA |
| 1213 | CCTAGTGACAAGTTTTTCA | 2955 | TGAAAAACTTGTCACTAGG |
| 1218 | TGACAAGTTTTTCACATCC | 2960 | GGATGTGAAAAACTTGTCA |
| 1219 | GACAAGTTTTTCACATCCC | 2961 | GGGATGTGAAAAACTTGTC |
| 1231 | ACATCCCATAATGGCATGC | 2973 | GCATGCCATTATGGGATGT |
| 1232 | CATCCCATAATGGCATGCA | 2974 | TGCATGCCATTATGGGATG |
| 1241 | ATGGCATGCAGTTCAGTAC | 2983 | GTACTGAACTGCATGCCAT |
| 1245 | CATGCAGTTCAGTACCTGG | 2987 | CCAGGTACTGAACTGCATG |
| 1250 | AGTTCAGTACCTGGGACAA | 2992 | TTGTCCCAGGTACTGAACT |
| 1252 | TTCAGTACCTGGGACAATG | 2994 | CATTGTCCCAGGTACTGAA |
| 1269 | TGACAATGATAAGTTTGAA | 3011 | TTCAAACTTATCATTGTCA |
| 1273 | AATGATAAGTTTGAAGGCA | 3015 | TGCCTTCAAACTTATCATT |
| 1279 | AAGTTTGAAGGCAACTGTG | 3021 | CACAGTTGCCTTCAAACTT |
| 1303 | CAGGATGGATCTGGTTGGT | 3045 | ACCAACCAGATCCATCCTG |
| 1305 | GGATGGATCTGGTTGGTGG | 3047 | CCACCAACCAGATCCATCC |
| 1327 | AACAAGTGTCACGCTGGCC | 3069 | GGCCAGCGTGACACTTGTT |
| 1329 | CAAGTGTCACGCTGGCCAT | 3071 | ATGGCCAGCGTGACACTTG |
| 1334 | GTCACGCTGGCCATCTCAA | 3076 | TTGAGATGGCCAGCGTGAC |
| 1355 | GAGTTTATTACCAAGGTGG | 3097 | CCACCTTGGTAATAAACTC |
| 1357 | GTTTATTACCAAGGTGGCA | 3099 | TGCCACCTTGGTAATAAAC |
| 1363 | TACCAAGGTGGCACTTACT | 3105 | AGTAAGTGCCACCTTGGTA |
| 1364 | ACCAAGGTGGCACTTACTC | 3106 | GAGTAAGTGCCACCTTGGT |
| 1367 | AAGGTGGCACTTACTCAAA | 3109 | TTTGAGTAAGTGCCACCTT |
| 1371 | TGGCACTTACTCAAAAGCA | 3113 | TGCTTTTGAGTAAGTGCCA |
| 1388 | CATCTACTCCTAATGGTTA | 3130 | TAACCATTAGGAGTAGATG |
| 1392 | TACTCCTAATGGTTATGAT | 3134 | ATCATAACCATTAGGAGTA |
| 1424 | GGGCCACTTGGAAAACCCG | 3166 | CGGGTTTTCCAAGTGGCCC |
| 1429 | ACTTGGAAAACCCGGTGGT | 3171 | ACCACCGGGTTTTCCAAGT |
| 1430 | CTTGGAAAACCCGGTGGTA | 3172 | TACCACCGGGTTTTCCAAG |
| 1433 | GGAAAACCCGGTGGTATTC | 3175 | GAATACCACCGGGTTTTCC |
| 1471 | AAGATAATCCCATTCAACA | 3213 | TGTTGAATGGGATTATCTT |
| 1472 | AGATAATCCCATTCAACAG | 3214 | CTGTTGAATGGGATTATCT |
| 1476 | AATCCCATTCAACAGACTC | 3218 | GAGTCTGTTGAATGGGATT |
| 1486 | AACAGACTCACAATTGGAG | 3228 | CTCCAATTGTGAGTCTGTT |
| 1489 | AGACTCACAATTGGAGAAG | 3231 | CTTCTCCAATTGTGAGTCT |
| 1516 | CACCACCTGGGGGGAGCCA | 3258 | TGGCTCCCCCCAGGTGGTG |
| 1518 | CCACCTGGGGGGAGCCAAA | 3260 | TTTGGCTCCCCCCAGGTGG |
| 1524 | GGGGGGAGCCAAACAGGCT | 3266 | AGCCTGTTTGGCTCCCCCC |
| 1525 | GGGGGAGCCAAACAGGCTG | 3267 | CAGCCTGTTTGGCTCCCCC |
| 1526 | GGGGAGCCAAACAGGCTGG | 3268 | CCAGCCTGTTTGGCTCCCC |
| 1534 | AAACAGGCTGGAGACGTTT | 3276 | AAACGTCTCCAGCCTGTTT |
| 1539 | GGCTGGAGACGTTTAAAAG | 3281 | CTTTTAAACGTCTCCAGCC |
| 1593 | TTATCTGAACAGAGAGATA | 3335 | TATCTCTCTGTTCAGATAA |
| 1594 | TATCTGAACAGAGAGATAT | 3336 | ATATCTCTCTGTTCAGATA |
| 1598 | TGAACAGAGAGATATAATA | 3340 | TATTATATCTCTCTGTTCA |
| 1600 | AACAGAGAGATATAATATT | 3342 | AATATTATATCTCTCTGTT |
| 1601 | ACAGAGAGATATAATATTT | 3343 | AAATATTATATCTCTCTGT |
| 1614 | ATATTTTTCCTATTGGACA | 3356 | TGTCCAATAGGAAAAATAT |
| 1616 | ATTTTTCCTATTGGACAAT | 3358 | ATTGTCCAATAGGAAAAAT |
| 1650 | CACTTCATTTTAAGAGCAA | 3392 | TTGCTCTTAAAATGAAGTG |
| 1693 | AACAGTTTTATGCTGATGA | 3435 | TCATCAGCATAAAACTGTT |
| 1703 | TGCTGATGATAATTTATCT | 3445 | AGATAAATTATCATCAGCA |

The siRNAs in subset G have the following characteristics:
Cross-reactivity: With 19mer in human FGG mRNA, with 17mer/19mer in NHP FGG
Specificity category: For human and NHP: AS2 or better, SS3 or better
miRNA seeds: AS+SS strand: seed region not conserved in human, mouse, and rat and not present in >4 species
Off-target frequency: ≤30 human off-targets matched with 2 mismatches in antisense strand
SNPs: siRNA target sites do not harbor SNPs with a MAF≥100 (pos. 2-18)

### Example 3: Screening FGG siRNAs for Activity in Hep 3B2.1-7 Cells in Culture

Chemically modified FGG siRNAs cross reactive for at least human and non-human primates will be assayed for FGG mRNA knockdown activity in cells in culture. Hep 3B2.1-7 cells (ATCC R catalog #HB-8064) will be seeded in 96-well tissue culture plates at a cell density of 7,500 cells per well in EMEM media (VWR catalog #76000-922) supplemented with 10% fetal bovine serum and incubated overnight in a water-jacketed, humidified incubator at 37° C. in an atmosphere without supplemental carbon dioxide. The FGG siRNAs will be individually transfected into Hep 3B2.1-7 cells in duplicate wells at 1 nM and 10 nM final concentration using 0.3 μL Lipofectamine RNAiMax (Fisher, catalog #13778150) in 5 uL Opti-MEM (Thermo Fisher, catalog #31985070) per well. Silencer Select Negative Control #3 (ThermoFisher, Catalog #4392420 ID s51788) will be transfected at 1 nM and 10 nM final concentrations as a control. A positive control siRNA (ThermoFisher, Catalog #) will be transfected at 1 nM and 10 nM final concentrations. After incubation for 48 hours at 37° C., total RNA will be harvested from each well and cDNA prepared using TaqMan® Fast Advanced Cells-to-CT™ Kit (ThermoFisher, catalog #A35374) according to the manufacturer's instructions. The level of FGG mRNA from each well will be measured in triplicate by biplex real-time qPCR on a QuantStudio 6 Pro instrument (Applied Biosystems) using TaqMan Gene Expression Assay for human FGG (ThermoFisher, assay #Hs00241037_m1). The level of PPIA mRNA will be measured using TaqMan Gene Expression Assay (ThermoFisher, assay #Hs99999904_m1) and used to determine relative FGG mRNA levels in each well using the delta-delta Ct method. All data will be normalized to relative FGG mRNA levels in untreated Hep 3B2.1-7 cells. Identification of siRNAs targeting FGG that reduce FGG expression is anticipated.

### Example 4: Determining the IC50 of FGG siRNAs

The IC50 values for knockdown of FGG mRNA by select FGG siRNAs will be determined in Hep 3B2.1-7 cells. The siRNAs will be assayed individually in triplicate at 30 nM, 10 nM, 3 nM, 1 nM and 0.3 nM, 0.1 nM and 0.03 nM. Hep 3B2.1-7 cells (ATCC® catalog #HB-8064) will be seeded in 96-well tissue culture plates at a cell density of 7,500 cells per well in EMEM media (VWR catalog #76000-922) supplemented with 10% fetal bovine serum and incubated overnight in a water-jacketed, humidified incubator at 37° C. in an atmosphere without supplemental carbon dioxide. The FGG siRNAs will be individually transfected using 0.3 μL Lipofectamine RNAiMax (Fisher, catalog #13778150) in 5 uL Opti-MEM (Thermo Fisher, catalog #31985070) per well. After incubation for 48 hours at 37° C., total RNA will be harvested from each well and cDNA prepared using TaqMan® Fast Advanced Cells-to-CT™ Kit (ThermoFisher, Catalog #A35374) according to the manufacturer's instructions. The level of FGG mRNA from each well will be measured in triplicate by biplex real-time qPCR on a QuantStudio 6 Pro instrument (Applied Biosystems) using TaqMan Gene Expression Assay for human FGG (ThermoFisher, assay #Hs00241037_m1). The level of PPIA mRNA will be measured using TaqMan Gene Expression Assay (ThermoFisher, assay #Hs99999904_m1) and used to determine relative FGG mRNA levels in each well using the delta-delta Ct method. All data will be normalized to relative FGG mRNA levels in untreated Hep 3B2.1-7 cells. Curve fit will be accomplish using the [inhibitor] vs. response (three parameters) function in GraphPad Prism software.

### Example 5: ASO-Mediated Knockdown of FGG in HEPG2 Cell Line

ASOs targeted to the FGG mRNA that downregulate levels of FGG mRNA leading to a decrease in FGG secretion, when administered to the cultured human hepatocyte cell line, HepG2.

On Day 0, the HEPG2 cells are seeded at 150,000 cells/mL into a Falcon 24-well tissue culture plate (ThermoFisher Cat. No. 353047) at 0.5 mL per well.

On Day 1, the FGG ASO and negative control ASO master mixes are prepared. The FGG ASO master mix contains 350 μL of Opti-MEM (ThermoFisher Cat. No. 4427037—s1288 Lot No. AS02B02D) and 3.5 ul of a FGG ASO (10 uM stock). The negative control ASO master mix contains 350 μL of Opti-MEM and 3.5 ul of negative control ASO (ThermoFisher Cat. No. 4390843, 10 uM stock). Next, 3 μL of TransIT-X2 (Mirus Cat. No. MIR-6000) is added to each master mix. The mixes are incubated for 15 minutes to allow transfection complexes to form, then 51 ul of the appropriate master mix+TransIT-X2 is added to duplicate wells of HEPG2 cells with a final ASO concentration of 10 nM.

On Day 3, 48 hours post transfection, media is collected and mixed with protein lysis buffer containing protease and phosphatase inhibitors, and the cells are lysed using the Cells-to-Ct kit according to the manufacturer's protocol (ThermoFisher Cat. No. 4399002). For the Cells-to-Ct, cells are washed with 50 ul using cold 1×PBS and lysed by adding 49.5 ul of Lysis Solution and 0.5 ul Dnase I per well and pipetting up and down 5 times and incubating for 5 minutes at room temperature. The Stop Solution (5 ul/well) is added to each well and mixed by pipetting up and down five times and incubating at room temperature for 2 minutes. The reverse transcriptase reaction is performed using 22.5 ul of the lysate according to the manufacturer's protocol. Samples are stored at −80° C. until real-time qPCR is performed in triplicate using TaqMan Gene Expression Assays (Applied Biosystems FAM/FGG using a BioRad CFX96 Cat. No. 1855195). For the protein quantification, equivalent quantities (30-50 μg) of protein are separated by 10% SDS polyacrylamide gels and transferred to polyvinylidene fluoride membranes. Membranes are blocked with 5% nonfat milk and incubated overnight with the appropriate primary antibody at dilutions specified by the manufacturer. Next, the membranes are washed three times in TBST and incubated with the corresponding horseradish peroxidase conjugated secondary antibody at 1:5,000 dilution for 1 hr. Bound secondary antibody is detected using an enhanced chemiluminescence system. The primary immunoblotting antibody is an anti-FGG antibody (Abcam, Cambridge, UK).

A decrease in FGG mRNA expression in the HEPG2 cells is expected after transfection with the FGG ASO compared to FGG mRNA levels in HEPG2 cells transfected with the non-specific control ASO 48 hours after transfection. There is an expected decrease in the amount of FGG secreted protein, measured by quantifying the amount of FGG protein in media of HEPG2 cells transfected with the FGG ASO relative to the amount of FGG protein in media of HEPG2 cells transfected with a non-specific control ASO 48 hours after transfection. These results show that the FGG ASOs elicit knockdown of FGG mRNA in HEPG2 cells and that the decrease in FGG expression is correlated with a decrease in FGG protein secretion.

### Example 6: Determining the Activity of Species Cross-Reactive siRNAs Targeting FGG in Mice

Five groups (n=4/group) of 8 week old male ICR mice (Harlan) were utilized in this study. On Study Day −4, all animals were anesthetized and blood was collected via the submandibular vein and into tubes containing citrate for collection of plasma. Plasma fibrinogen levels were measured use the Clauss method (IDEXX Laboratories, Test #6308) and by ELISA according to the manufacturer's instructions (Molecular Innovations Catalog #MFBGNKT). On Study Day 0, Group 1 mice were injected subcutaneously with 100 μL of sterile PBS, Group 2 mice were subcutaneously injected with 200 μg of ETD01592 (sense strand SEQ ID NO: 3591; antisense strand SEQ ID NO: 3595) in 100 μL of sterile PBS, Group 3 mice were subcutaneously injected with 200 ug ETD01593 (sense strand SEQ ID NO: 3592; antisense strand SEQ ID NO: 3596) in 100 μL of sterile PBS, Group 4 mice were subcutaneously injected with 200 μg of ETD01594 (sense strand SEQ ID NO: 3593; antisense strand SEQ ID NO: 3597) in 100 uL PBS, and Group 5 mice were subcutaneously injected with 200 μg of ETD01595 (sense strand SEQ ID NO: 3594; antisense strand SEQ ID NO: 3598) in 100 uL PBS. On Study Day 10, the animals from all Groups were anesthetized, bled via cardiac puncture to collect serum and plasma, and then euthanized. A liver sample was collected from all animals and placed in RNAlater™ Stabilization Solution (Thermo Fisher, Catalog #AM7020). Serum clinical chemistry analyses were performed (IDEXX Laboratories, Test #60513) and plasma fibrinogen levels were measured as described for the Day −4 samples. The liver samples were processed in homogenization buffer (Maxwell RSC simplyRNA Tissue Kit) using Soft Tissue Homogenizing Kit CK14 (Bertin Instruments, catalog #P000933-LYSK0-A) in a Percellys 24 tissue homogenizer (Bertin Instruments) set at 5000 rpm for two 10 second cycles. Total RNA from the liver lysate was purified on a Maxwell RSC 48 platform (Promega Corporation) according to the manufacturer's recommendations. The relative level of FGG mRNA in each liver sample was assessed by RT-qPCR on a QuantStudio 6 Pro instrument (Applied Biosystems) using TaqMan assays for mouse FGG (ThermoFisher, assay #Mm00513575 ml) and the mouse housekeeping gene PPIA (ThermoFisher, assay #Mm02342430_g1), and then normalized to the mean value of the control mice (Group 1) using the delta-delta Ct method.

The results of the liver mRNA analyses are shown in Table 9. Animals treated ETD01592 (Group 2), ETD01593 (Group 3), ETD01594 (Group 4), or ETD01595 (Group 4) showed decreased liver FGG mRNA levels compared with mice injected with PBS (Group 1). The results of the plasma fibrinogen analyses are shown in Table 10. Animals treated with ETD01592 (Group 2), ETD01593 (Group 3), ETD01594 (Group 4), or ETD01595 (Group 5) showed decreased plasma fibrinogen levels as measured by the Clauss method or by ELISA compared with mice injected with PBS (Group 1). The results from the clinical chemistry indicated all the siRNAs were generally well tolerated (Table 11).

TABLE 9
Day 10 FGG mRNA liver levels in mice
treated with siRNAs targeting FGG

| | | | Relative Liver | |
|---|---|---|---|---|
| | | | FGG mRNA | |
| Group # | Treatment | Mouse # | Level | Mean |
| 1 | PBS | 1 | 0.933 | 1.00 |
| | | 2 | 1.229 | |
| | | 3 | 0.873 | |
| | | 4 | 0.998 | |
| 2 | ETD01592 | 5 | 0.098 | 0.13 |
| | | 6 | 0.165 | |
| | | 7 | 0.093 | |
| | | 8 | 0.166 | |
| 3 | ETD01593 | 9 | 0.201 | 0.22 |
| | | 10 | 0.124 | |
| | | 11 | 0.293 | |
| | | 12 | 0.316 | |
| 4 | ETD01594 | 13 | 0.148 | 0.14 |
| | | 14 | 0.121 | |
| | | 15 | 0.143 | |
| | | 16 | 0.132 | |
| 5 | ETD01595 | 17 | 0.095 | 0.16 |
| | | 18 | 0.158 | |
| | | 19 | 0.172 | |
| | | 20 | 0.27 | |

TABLE 10
Day 10 plasma fibrinogen levels in mice treated with siRNAs targeting FGG

| | | | Fibrinogen Plasma | Relative Plasma | |
|---|---|---|---|---|---|
| | | | Level (Clauss | Fibrinogen | |
| Group # | Treatment | Mouse # | method, mg/dL)* | Level (ELISA) | Mean |
| 1 | PBS | 1 | 2.840 | 1.21 | 1.00 |
| | | 2 | 3.250 | 1.04 | |
| | | 3 | 2.080 | 0.85 | |
| | | 4 | 2.550 | 0.90 | |
| 2 | ETD01592 | 5 | <LLOQ | 0.01 | 0.07 |
| | | 6 | <LLOQ | 0.10 | |
| | | 7 | <LLOQ | 0.09 | |
| | | 8 | <LLOQ | 0.07 | |
| 3 | ETD01593 | 9 | <LLOQ | 0.18 | 0.27 |
| | | 10 | <LLOQ | 0.11 | |
| | | 11 | <LLOQ | 0.52 | |
| | | 12 | 0.490 | 0.27 | |
| 4 | ETD01594 | 13 | <LLOQ | 0.13 | 0.14 |
| | | 14 | <LLOQ | 0.11 | |
| | | 15 | <LLOQ | 0.13 | |
| | | 16 | <LLOQ | 0.20 | |
| 5 | ETD01595 | 17 | <LLOQ | 0.20 | 0.23 |
| | | 18 | <LLOQ | 0.16 | |
| | | 19 | <LLOQ | 0.16 | |
| | | 20 | <LLOQ | 0.39 | |

*Clauss method LLOQ <0.5 mg/dL

TABLE 11
Clinical chemistry results after injection of mice with 200 ug of
ETD01592, ETD01593, ETD01594 or ETD01595

| Group | | Mouse | ALP | AST | ALT | BUN | CREAT |
|---|---|---|---|---|---|---|---|
| # | Treatment | # | (U/L) | (U/L) | (U/L) | (mg/dL) | (mg/dL) |
| 1 | PBS | 1 | 79 | 122 | 42 | 31 | 0.2 |
| | | 2 | 91 | 61 | 39 | 25 | 0.2 |
| | | 3 | 64 | 141 | 66 | 34 | 0.2 |
| | | 4 | 86 | 309 | 152 | 32 | 0 |
| 2 | ETD01592 | 5 | 69 | 157 | 47 | 27 | 0.2 |
| | | 6 | 74 | 90 | 76 | 29 | 0.2 |
| | | 7 | 51 | 83 | 38 | 26 | 0.2 |
| | | 8 | 102 | 91 | 40 | 27 | 0.2 |
| 3 | ETD01593 | 9 | 100 | 174 | 46 | 29 | 0.2 |
| | | 10 | 74 | 185 | 84 | 25 | 0.1 |
| | | 11 | 103 | 78 | 63 | 27 | 0.2 |
| | | 12 | 98 | 41 | 22 | 22 | 0.2 |
| 4 | ETD01594 | 13 | 111 | 333 | 177 | 32 | 0.1 |
| | | 14 | 61 | 53 | 22 | 27 | 0.2 |
| | | 15 | 57 | 102 | 48 | 32 | 0.2 |
| | | 16 | 105 | 486 | 107 | 28 | 0.2 |
| 5 | ETD01595 | 17 | 114 | 129 | 43 | 33 | 0.2 |
| | | 18 | 86 | 145 | 71 | 29 | 0.1 |
| | | 19 | 80 | 170 | 95 | 28 | 0 |
| | | 20 | 102 | 79 | 34 | 28 | 0.2 |

### Example 7: Determining the Activity of siRNAs Targeting FGG in Mice at Low Dose Levels

Nine groups (n=3/group) of 8 week old male ICR mice (Harlan) were utilized in this study. On Study Day 0, mice in Group 1 were injected subcutaneously with 100 μL of sterile PBS, mice in Groups 2 and 3 were subcutaneously injected with 20 μg or 60 μg of ETD01592, respectively (sense strand SEQ ID NO: 3591; antisense strand SEQ ID NO: 3595) in 100 μL of sterile PBS, mice in Groups 4 and 5 were subcutaneously injected with 20 μg or 60 μg of ETD01593, respectively (sense strand SEQ ID NO: 3592; antisense strand SEQ ID NO: 3596) in 100 μL of sterile PBS, mice in Groups 6 and 7 were subcutaneously injected with 20 μg or 60 μg of ETD01594, respectively (sense strand SEQ ID NO: 3593; antisense strand SEQ ID NO: 3597) in 100 uL PBS, and mice in Groups 8 and 9 were subcutaneously injected with 20 μg or 60 μg of ETD01595, respectively (sense strand SEQ ID NO: 3594; antisense strand SEQ ID NO: 3598) in 100 uL PBS. On Study Day 10, the animals from all groups were anesthetized, bled via cardiac puncture to collect serum and plasma, and then euthanized. A liver sample was collected from all animals and placed in RNAlater™ Stabilization Solution (Thermo Fisher, Catalog #AM7020). Plasma fibrinogen levels were measured by ELISA according to the manufacturer's instructions (Molecular Innovations Catalog #MFBGNKT). Plasma prothrombin time (PT) and activated partial thromboplastin time (aPTT) (IDEXX Laboratories, Test #6308) and serum clinical chemistry measurements were also performed (IDEXX Laboratories, Test #60513). The liver samples were processed in homogenization buffer (Maxwell RSC simplyRNA Tissue Kit) using Soft Tissue Homogenizing Kit CK14 (Bertin Instruments, catalog #P000933-LYSK0-A) in a Percellys 24 tissue homogenizer (Bertin Instruments) set at 5000 rpm for two 10 second cycles. Total RNA from the liver lysate was purified on a Maxwell RSC 48 platform (Promega Corporation) according to the manufacturer's recommendations. The relative level of FGG mRNA in each liver sample was assessed by RT-qPCR on a QuantStudio 6 Pro instrument (Applied Biosystems) using TaqMan assays for mouse FGG (ThermoFisher, assay #Mm00513575 ml) and the mouse housekeeping gene PPIA (ThermoFisher, assay #Mm02342430_g1), and then normalized to the mean value of the control mice (Group 1) using the delta-delta Ct method.

The results of the liver mRNA analyses are shown in Table 12. Animals treated with 20 ug ETD01592, ETD01593, ETD01594, or ETD01595 showed decreased liver FGG mRNA levels compared with mice injected with PBS. Animals treated with 60 ug ETD01592, ETD01593, ETD01594, or ETD1595 showed decreased liver FGG mRNA levels compared with mice injected with 20 ug of those siRNAs or with mice injected with PBS. The results of the plasma fibrinogen ELISA are shown in Table 13. Animals treated with 20 ug ETD01592, ETD01593, ETD01594, or ETD01595 showed decreased plasma fibrinogen protein levels compared with mice injected with PBS. Animals treated with 60 ug ETD01592, ETD01593, ETD01594, or ETD01595 showed decreased plasma fibrinogen protein levels compared with mice injected with 20 μg of those siRNAs or with mice injected with PBS. The results of the PT and aPTT measurements in animals treated with 20 ug and 60 ug ETD01592, ETD01593, ETD01594, or ETD01595 are shown in Table 14. The results from the clinical chemistry indicate that all the siRNAs were generally well tolerated at these dose levels (Table 15).

TABLE 12
FGG mRNA liver levels in mice treated with 20 ug or
60 ug of ETD01592, ETD01593, ETD01594 or ETD01595.

| | | | | Relative Liver | |
|---|---|---|---|---|---|
| | | Dose | | FGG mRNA | |
| Group # | Treatment | Level | Mouse # | Level | Mean |
| 1 | PBS | NA | 1 | 0.97 | 1.00 |
| | | | 2 | 1.07 | |
| | | | 3 | 0.97 | |
| 2 | ETD01592 | 20 ug | 4 | 0.90 | 0.80 |
| | | | 5 | 0.58 | |
| | | | 6 | 0.93 | |
| 3 | ETD01592 | 60 ug | 7 | 0.36 | 0.54 |
| | | | 8 | 0.57 | |
| | | | 9 | 0.70 | |
| 4 | ETD01593 | 20 ug | 10 | 1.10 | 1.12 |
| | | | 11 | 0.87 | |
| | | | 12 | 1.38 | |
| 5 | ETD01593 | 60 ug | 13 | 0.61 | 0.54 |
| | | | 14 | 0.42 | |
| | | | 15 | 0.58 | |
| 6 | ETD01594 | 20 ug | 16 | 0.69 | 0.78 |
| | | | 17 | 0.65 | |
| | | | 18 | 1.00 | |
| 7 | ETD01594 | 60 ug | 19 | 0.29 | 0.26 |
| | | | 20 | 0.36 | |
| | | | 21 | 0.11 | |
| 8 | ETD01595 | 20 ug | 22 | 0.64 | 0.86 |
| | | | 23 | 0.84 | |
| | | | 24 | 1.11 | |
| 9 | ETD01595 | 60 ug | 25 | 0.60 | 0.63 |
| | | | 26 | 0.58 | |
| | | | 27 | 0.71 | |

TABLE 13
Plasma fibrinogen levels in mice treated with 20 ug
or 60 ug of ETD01592, ETD01593, ETD01594 or ETD01595.

| | | | | Relative Plasma | |
|---|---|---|---|---|---|
| | | Dose | | Fibrinogen | |
| Group # | Treatment | Level | Mouse # | Level (ELISA) | Mean |
| 1 | PBS | NA | 1 | 1.38 | 1.00 |
| | | | 2 | 1.00 | |
| | | | 3 | 0.62 | |
| 2 | ETD01592 | 20 ug | 4 | 0.83 | 0.61 |
| | | | 5 | 0.51 | |
| | | | 6 | 0.49 | |
| 3 | ETD01592 | 60 ug | 7 | 0.13 | 0.20 |
| | | | 8 | 0.21 | |
| | | | 9 | 0.27 | |
| 4 | ETD01593 | 20 ug | 10 | 0.53 | 0.54 |
| | | | 11 | 0.54 | |
| | | | 12 | 0.55 | |
| 5 | ETD01593 | 60 ug | 13 | 0.32 | 0.30 |
| | | | 14 | 0.46 | |
| | | | 15 | 0.11 | |
| 6 | ETD01594 | 20 ug | 16 | 0.50 | 0.28 |
| | | | 17 | 0.29 | |
| | | | 18 | 0.04 | |
| 7 | ETD01594 | 60 ug | 19 | 0.23 | 0.15 |
| | | | 20 | 0.20 | |
| | | | 21 | 0.03 | |
| 8 | ETD01595 | 20 ug | 22 | 0.63 | 0.58 |
| | | | 23 | 0.64 | |
| | | | 24 | 0.47 | |
| 9 | ETD01595 | 60 ug | 25 | 0.45 | 0.45 |
| | | | 26 | 0.39 | |
| | | | 27 | 0.51 | |

TABLE 14
PTT and aPTT in mice treated with 20 ug or 60 ug of
ETD01592, ETD01593, ETD01594 or ETD01595.

| Group | | Dose | Mouse | PT | Mean | aPTT | Mean |
|---|---|---|---|---|---|---|---|
| # | Treatment | Level | # | (s) | (s) | (s) | (s) |
| 1 | PBS | NA | 1 | 11.6 | 11.4 | 23.9 | 28.0 |
| | | | 2 | 11.3 | | 33.6 | |
| | | | 3 | 11.3 | | 26.4 | |
| 2 | ETD01592 | 20 ug | 4 | 11.7 | 11.6 | 26.1 | 29.7 |
| | | | 5 | 11.5 | | 33.8 | |
| | | | 6 | 11.6 | | 29.2 | |
| 3 | ETD01592 | 60 ug | 7 | 14.1 | 13.4 | 22.5 | 24.8 |
| | | | 8 | 13.3 | | 25.9 | |
| | | | 9 | 12.8 | | 26 | |
| 4 | ETD01593 | 20 ug | 10 | 11.7 | 12.1 | 30.6 | 32.0 |
| | | | 11 | 12.1 | | 24.2 | |
| | | | 12 | 12.5 | | 41.3 | |
| 5 | ETD01593 | 60 ug | 13 | 11.4 | 12.8 | 20.3 | 33.3 |
| | | | 14 | 12.3 | | 50.9 | |
| | | | 15 | 14.8 | | 28.8 | |
| 6 | ETD01594 | 20 ug | 16 | 11.3 | 15.3 | 25.8 | 27.9 |
| | | | 17 | 12.2 | | 27.8 | |
| | | | 18 | 22.3 | | 30.1 | |
| 7 | ETD01594 | 60 ug | 19 | 17.6 | 19.8 | 23.9 | 47.2 |
| | | | 20 | 13.2 | | 26.4 | |
| | | | 21 | 28.5 | | 91.3 | |
| 8 | ETD01595 | 20 ug | 22 | 11.6 | 11.8 | 19.5 | 23.4 |
| | | | 23 | 12.7 | | 30.8 | |
| | | | 24 | 10.9 | | 19.9 | |
| 9 | ETD01595 | 60 ug | 25 | 11.7 | 11.9 | 21.4 | 25.8 |
| | | | 26 | 11.4 | | 29.4 | |
| | | | 27 | 12.5 | | 26.5 | |

TABLE 15
Clinical chemistry results after injection of mice with 20 ug or
60 ug of ETD01592, ETD01593, ETD01594 or ETD01595.

| | | | | | | | BUN | CREAT |
|---|---|---|---|---|---|---|---|---|
| Group | | Dose | Mouse | ALP | AST | ALT | (mg/ | (mg/ |
| # | Treatment | Level | # | (U/L) | (U/L) | (U/L) | dL) | dL) |
| 1 | PBS | NA | 1 | 77 | 83 | 32 | 22 | 0.1 |
| | | | 2 | 109 | 70 | 27 | 21 | 0.2 |
| | | | 3 | 118 | 65 | 30 | 22 | 0.1 |
| 2 | ETD01592 | 20 ug | 4 | 138 | 54 | 26 | 24 | 0.1 |
| | | | 5 | 93 | 55 | 26 | 25 | 0.1 |
| | | | 6 | 107 | 124 | 54 | 22 | 0.1 |
| 3 | ETD01592 | 60 ug | 7 | 100 | 67 | 25 | 18 | 0.1 |
| | | | 8 | 105 | 56 | 24 | 20 | 0.1 |
| | | | 9 | 69 | 64 | 23 | 19 | 0.1 |
| 4 | ETD01593 | 20 ug | 10 | 71 | 174 | 89 | 25 | 0.1 |
| | | | 11 | 99 | 67 | 31 | 22 | 0.1 |
| | | | 12 | 117 | 146 | 70 | 23 | 0.2 |
| 5 | ETD01593 | 60 ug | 13 | 110 | 69 | 30 | 27 | 0.2 |
| | | | 14 | 133 | 100 | 30 | 24 | 0.2 |
| | | | 15 | 119 | 46 | 26 | 31 | 0.1 |
| 6 | ETD01594 | 20 ug | 16 | 124 | 51 | 26 | 22 | 0.1 |
| | | | 17 | 126 | 235 | 158 | 24 | 0.1 |
| | | | 18 | 171 | 90 | 38 | 24 | 0.2 |
| 7 | ETD01594 | 60 ug | 19 | 72 | 54 | 32 | 20 | 0.1 |
| | | | 20 | 100 | 56 | 35 | 23 | 0.2 |
| | | | 21 | 114 | 135 | 50 | 31 | 0.2 |
| 8 | ETD01595 | 20 ug | 22 | 120 | 77 | 38 | 22 | 0.1 |
| | | | 23 | 93 | 387 | 102 | 28 | 0.2 |
| | | | 24 | 94 | 144 | 130 | 19 | 0.2 |
| 9 | ETD01595 | 60 ug | 25 | 93 | 72 | 23 | 21 | 0.1 |
| | | | 26 | 136 | 72 | 57 | 18 | 0.1 |
| | | | 27 | 135 | 51 | 22 | 25 | 0.2 |

### Example 8: Inhibition of FGG in a Mouse Model for Depression Using Modified FGG siRNAs or ASOs

In this experiment, a mouse model of depression is used to evaluate the effect of siRNA or ASO-mediated inhibition of FGG. To induce depression like symptoms the mice will be subjected to Chronic Social Defeat (CSD) by repeated social confrontations with an aggressive mouse for 15 consecutive days. Depression like symptoms are measured using Open Field Test, elevated T-maze and Tail Suspension Test.

Briefly, C57Bl/6J mice (Charles River, MA USA) are divided into six groups: Group 1—a group treated with non-targeting control siRNA, Group 2—a group treated with non-targeting control ASO, Group 3—a group treated with FGG siRNA, Group 4—a group treated with FGG ASO, Group 5—a group treated with vehicle, Group 6—a group not subjected to chronic social defeat, treated with vehicle. Each group contains 20 male mice.

Administration of siRNA or ASO is achieved with a 100 ul subcutaneous injection of naked siRNA or ASO resuspended at concentration of 10 mg/mL in PBS. On Study Days 0, 7 and 21, Group 1 mice will be injected subcutaneously with non-targeting control siRNA, Group 2 mice will be injected subcutaneously with non-targeting control ASO, Group 3 mice will be injected subcutaneously with siRNA targeting mouse FGG, Group 4 mice will be injected subcutaneously with ASO targeting mouse FGG, and Group 5 and Group 6 mice will be injected subcutaneously with PBS.

All mice from groups 1-5 are exposed to CD-1/ICR mice (Charles River, MA USA), that have been previously screened for exhibiting aggressive behavior, for 15 days total beginning on Study Day 14. The behavioral tests are performed in Groups 1-5, 8 days after the final injection (Study Day 29).

Mice are first evaluated using the open field paradigm (44×44×40 cm) in a sound-attenuated room. The total distance (cm) traveled by each mouse is recorded for 5 min by a video surveillance system (SMART; Panlab SL, Barcelona, Spain) and is used to quantify activity levels. The floor of the open-field apparatus is cleaned with 10% ethanol between tests.

The elevated T-maze is a behavioral test useful for screening potential antidepressant drugs and assessing other manipulations that are expected to affect anxiety related behaviors. Mice are placed individually in an apparatus that consists of three elevated arms, one enclosed and two open. Mice will be initially placed in the enclosed arm of the maze and the time taken to leave the enclosed arm in three consecutive trials is measured. The total time in enclosed is recorded as an index of anxiety-like behavior.

The tail suspension test is a behavioral test useful for screening potential antidepressant drugs and assessing other manipulations that are expected to affect depression related behaviors. Mice are suspended by their tail, without the ability to escape or reach the sides of the enclosure. During the duration of the test, 6 minutes, the mouse's escape-oriented behaviors will be quantified as well as time spend immobile. The total time spent attempting to escape versus time spent immobile is recorded as an index of depressive-like behavior.

24 hours after the behavioral assessment, the mice are sacrificed by cervical dislocation following an intraperitoneal injection of 0.3 ml Nembutal (5 mg/ml) (Sigma Cat. No. 1507002). A liver sample will be collected from all animals and placed in RNAlater™ Stabilization Solution (Thermo Fisher, Catalog #AM7020). The liver samples will be processed in homogenization buffer (Maxwell RSC simplyRNA Tissue Kit) using Soft Tissue Homogenizing Kit CK14 (Bertin Instruments, catalog #P000933-LYSK0-A) in a Percellys 24 tissue homogenizer (Bertin Instruments) set at 5000 rpm for two 10 second cycles. Total RNA from the liver lysate was purified on a Maxwell RSC 48 platform (Promega Corporation) according to the manufacturer's recommendations. The relative level of FGG mRNA in each liver sample was assessed by RT-qPCR on a QuantStudio 6 Pro instrument (Applied Biosystems) using TaqMan assays for mouse FGG (ThermoFisher, assay #Mm00513575_m1) and the mouse housekeeping gene PPIA (ThermoFisher, assay #Mm02342430_g1), and then normalized to the mean value of the control mice using the delta-delta Ct method. Plasma fibrinogen levels will be measured use the Clauss method or by ELISA according to the manufacturer's instructions (Molecular Innovations Catalog #MFBGNKT).

A decrease in FGG mRNA expression in the liver tissue from mice dosed with the FGG siRNA or ASO is expected compared to FGG mRNA levels in the liver tissue from mice dosed with the non-specific controls. Measurement of plasma fibrinogen levels is expected to show a decrease in fibrinogen in the mice dosed with the FGG siRNA or ASO compared to fibrinogen from the plasma from mice dosed with non-specific control. There is an expected decrease in the time before the mice leave the enclosed arm of the elevated T-maze as well in a decrease in time spent in the enclosed arm in mice that receive the FGG siRNA or ASO compared to mice that receive non-specific control. In addition, there is an expected decrease in total immobility time in the tail suspension test along with no change in locomotor activity in the open field test in mice that receive the FGG siRNA or ASO compared to mice that receive non-specific control.

### Example 9: Inhibition of FGG in a Mouse Model for Alzheimer's Disease Using FGG siRNAs or ASOs

In this experiment, a mouse model of Alzheimer's disease using 5×FAD mice which express human APP and PSEN1 transgenes with a total of five AD-linked mutations is used to evaluate the effect of siRNA or ASO inhibition of FGG. Cognitive function is measured using a contextual fear conditioning (CFC).

Briefly, 7-month-old 5×FAD mice are divided into five groups: Group 1—a group treated with non-targeting control siRNA, Group 2—a group treated with non-targeting control ASO, Group 3—a group treated with FGG siRNA1, Group 4—a group treated with FGG ASO1, Group 5—a group treated with vehicle. Each group contains 20 mice.

Administration of siRNA or ASO is achieved with a 100 ul subcutaneous injection of GalNAc-conjugated siRNA or ASO at concentration of 10 mg/mL in PBS. On Study Days 0, 7 and 14, Group 1 mice will be injected subcutaneously with non-targeting control siRNA, Group 2 mice will be injected subcutaneously with non-targeting control ASO, Group 3 mice will be injected subcutaneously with siRNA1 targeting mouse FGG, Group 4 mice will be injected subcutaneously with ASO1 targeting mouse FGG, and Group 5 mice will be injected subcutaneously with vehicle. The behavioral tests are performed 7 days after the final injection.

To rule out nonspecific motor effects that could influence the results of the cognitive function tests, the potential effect of siRNA or ASO treatment on locomotor activity is assessed. Mice are evaluated using the openfield paradigm (44×44×40 cm) in a sound-attenuated room. The total distance (cm) traveled by each mouse is recorded for 5 min by a video surveillance system (SMART; Panlab SL, Barcelona, Spain) and is used to quantify activity levels. The floor of the open-field apparatus is cleaned with 10% ethanol between tests.

Mice are then evaluated using the contextual fear conditioning (CFC) and active avoidance (AA) paradigms. Mice are subjected to repeated electric shock stimuli in a sound-attenuated room over multiple trials. The freezing and avoidance behaviors are recorded for each trial by a video surveillance system (SMART; Panlab SL, Barcelona, Spain) and are used to quantify freezing time and avoidance. The floor of the apparatus is cleaned with 10% ethanol between tests.

24 hours after the behavioral assessment, the mice are sacrificed by cervical dislocation following an intraperitoneal injection of 0.3 ml Nembutal (5 mg/ml) (Sigma Cat. No. 1507002). A liver sample will be collected from all animals and placed in RNAlater™ Stabilization Solution (Thermo Fisher, Catalog #AM7020). The liver samples will be processed in homogenization buffer (Maxwell RSC simplyRNA Tissue Kit) using Soft Tissue Homogenizing Kit CK14 (Bertin Instruments, catalog #P000933-LYSK0-A) in a Percellys 24 tissue homogenizer (Bertin Instruments) set at 5000 rpm for two 10 second cycles. Total RNA from the liver lysate was purified on a Maxwell RSC 48 platform (Promega Corporation) according to the manufacturer's recommendations. The relative level of FGG mRNA in each liver sample was assessed by RT-qPCR on a QuantStudio 6 Pro instrument (Applied Biosystems) using TaqMan assays for mouse FGG (ThermoFisher, assay #Mm00513575_m1) and the mouse housekeeping gene PPIA (ThermoFisher, assay #Mm02342430_g1), and then normalized to the mean value of the control mice using the delta-delta Ct method. Plasma fibrinogen levels will be measured use the Clauss method or by ELISA according to the manufacturer's instructions (Molecular Innovations Catalog #MFBGNKT).

A decrease in FGG mRNA expression in the liver tissue from mice dosed with the FGG siRNA or ASO is expected compared to FGG mRNA levels in the liver tissue from mice dosed with the non-specific controls. Measurement of plasma fibrinogen levels is expected to show a decrease in fibrinogen in the mice dosed with the FGG siRNA or ASO compared to fibrinogen from the plasma from mice dosed with non-specific control. There is an expected decrease in freezing time in the CFC and increase in the avoidance behaviors in the AA in mice that receive the FGG siRNA or ASO compared to mice that receive the non-specific controls along with no change between treatment groups in the locomotor activity test.

### Example 10: Screening FGG siRNAs for Activity in Huh7 Cells in Culture

Chemically modified FGG siRNAs cross-reactive for at least human and non-human primates were assayed for FGG mRNA knockdown activity in cells in culture. Huh7 cells (Xenotech catalog #JCRB0403) were seeded in 96-well tissue culture plates at a cell density of 20,000 cells per well in DMEM media (VWR catalog #02-0100-0500) supplemented with 10% fetal bovine serum and incubated overnight in a water-jacketed, humidified incubator at 37° C. in an atmosphere containing 5% carbon dioxide. The FGG siRNAs were individually transfected into Huh7 cells in duplicate wells at 1 nM and 10 nM final concentration using 0.2 μL Lipofectamine RNAiMax (Fisher, catalog #13778150) in 5 uL Opti-MEM (Thermo Fisher, catalog #31985070) per well. Silencer Select Negative Control #1 (ThermoFisher, catalog #4390843) was transfected at 1 nM and 10 nM final concentrations as a negative control. Positive control siRNAs targeting FGG (ThermoFisher, catalog #4392420, Assay IDs s5179, s5180) were transfected at 1 nM and 10 nM final concentrations. After incubation for 48 hours at 37° C., total RNA was harvested from each well using TaqMan® Fast Advanced Cells-to-CT™ Kit (ThermoFisher, catalog #A35374) according to the manufacturer's instructions. Preparation of cDNA was performed using Quanta qScript cDNA SuperMix (VWR, Catalog #95048-500) according to the manufacturer's instructions. The level of FGG mRNA from each well was measured in triplicate by biplex real-time qPCR on a QuantStudio 6 Pro instrument (Applied Biosystems) using TaqMan® Fast Advanced Master Mix (Fisher Scientific catalog #44-445-58), TaqMan Gene Expression Assay for human FGG (ThermoFisher, assay #Hs00241037_m1) and TaqMan Gene Expression Assay for human PPIA (ThermoFisher, assay #Hs99999904_m1). The relative FGG mRNA levels in each well was calculated using the delta-delta Ct method. All data were normalized to relative FGG mRNA levels in untreated Huh7 cells. Results are shown in Table 16.

TABLE 16
Knockdown activity of FGG-specific siRNAs
at 1 nM and 10 nM in Huh7 cells

| | | | Relative Activity | |
|---|---|---|---|---|
| Untreated Cells | — | — | 1.00 | |
| siRNA | Sense Strand | Antisense Strand | 10 nM | 1 nM |
| name | SEQ ID NO | SEQ ID NO | siRNA | siRNA |
| Neg. Ctrl siRNA | — | — | 0.96 | 1.00 |
| Pos. Ctrl siRNA-1 | — | — | 0.09 | 0.34 |
| Pos. Ctrl siRNA-2 | — | — | 0.13 | 0.58 |
| ETD01663 | 3485 | 3538 | 0.19 | 0.51 |
| ETD01664 | 3486 | 3539 | 0.21 | 0.56 |
| ETD01665 | 3487 | 3540 | 0.18 | 0.41 |
| ETD01666 | 3488 | 3541 | 0.15 | 0.66 |
| ETD01667 | 3489 | 3542 | 0.64 | 0.87 |
| ETD01668 | 3490 | 3543 | 0.32 | 0.66 |
| ETD01669 | 3491 | 3544 | 0.39 | 0.63 |
| ETD01670 | 3492 | 3545 | 0.10 | 0.47 |
| ETD01671 | 3493 | 3546 | 0.65 | 0.73 |
| ETD01672 | 3494 | 3547 | 0.36 | 0.98 |
| ETD01673 | 3495 | 3548 | 0.46 | 0.80 |
| ETD01674 | 3496 | 3549 | 0.26 | 0.92 |
| ETD01675 | 3497 | 3550 | 0.48 | 0.86 |
| ETD01676 | 3498 | 3551 | 0.49 | 0.92 |
| ETD01677 | 3499 | 3552 | 0.35 | 0.89 |
| ETD01678 | 3500 | 3553 | 0.36 | 0.69 |
| ETD01679 | 3501 | 3554 | 0.44 | 0.95 |
| ETD01680 | 3502 | 3555 | 0.31 | 0.96 |
| ETD01681 | 3503 | 3556 | 0.26 | 0.71 |
| ETD01682 | 3504 | 3557 | 0.70 | 0.97 |
| ETD01683 | 3505 | 3558 | 0.32 | 0.87 |
| ETD01684 | 3506 | 3559 | 0.52 | 0.87 |
| ETD01685 | 3507 | 3560 | 0.27 | 0.87 |
| ETD01686 | 3508 | 3561 | 0.14 | 0.51 |
| ETD01687 | 3509 | 3562 | 0.47 | 0.85 |
| ETD01688 | 3510 | 3563 | 0.45 | 0.94 |
| ETD01689 | 3511 | 3564 | 0.97 | 0.74 |
| ETD01690 | 3512 | 3565 | 0.32 | 0.90 |
| ETD01691 | 3513 | 3566 | 0.52 | 0.98 |
| ETD01692 | 3514 | 3567 | 0.47 | 0.89 |
| ETD01693 | 3515 | 3568 | 0.42 | 0.92 |
| ETD01694 | 3516 | 3569 | 0.05 | 0.50 |
| ETD01695 | 3517 | 3570 | 0.57 | 0.84 |
| ETD01696 | 3518 | 3571 | 0.27 | 0.97 |
| ETD01697 | 3519 | 3572 | 0.20 | 0.65 |
| ETD01698 | 3520 | 3573 | 0.25 | 1.12 |
| ETD01699 | 3521 | 3574 | 0.39 | 0.95 |
| ETD01700 | 3522 | 3575 | 0.34 | 0.89 |
| ETD01701 | 3523 | 3576 | 0.16 | 0.84 |
| ETD01702 | 3524 | 3577 | 0.33 | 0.63 |
| ETD01703 | 3525 | 3578 | 0.19 | 0.52 |
| ETD01704 | 3526 | 3579 | 0.25 | 0.88 |
| ETD01705 | 3527 | 3580 | 0.31 | 0.79 |
| ETD01706 | 3528 | 3581 | 0.99 | 1.02 |
| ETD01707 | 3529 | 3582 | 0.26 | 0.89 |
| ETD01708 | 3530 | 3583 | 0.80 | 0.75 |
| ETD01709 | 3531 | 3584 | 0.26 | 0.81 |
| ETD01710 | 3532 | 3585 | 0.17 | 0.64 |
| ETD01711 | 3533 | 3586 | 0.44 | 0.41 |
| ETD01712 | 3534 | 3587 | 0.46 | 0.64 |
| ETD01713 | 3535 | 3588 | 0.23 | 0.68 |
| ETD01714 | 3536 | 3589 | 0.45 | 0.80 |
| ETD01715 | 3537 | 3590 | 0.45 | 0.94 |

### Example 11: Determining the IC50 of FGG siRNAs in Huh7 Cells in Culture

The IC50 values for knockdown of FGG mRNA by select FGG siRNAs were determined in Huh7 cells. The siRNAs were assayed individually in triplicate at 30 nM, 10 nM, 3 nM, 1 nM and 0.3 nM, 0.1 nM and 0.03 nM. Huh7 cells (Xenotech catalog #JCRB0403) were seeded in 96-well tissue culture plates at a cell density of 20,000 cells per well in DMEM media (VWR catalog #02-0100-0500) supplemented with 10% fetal bovine serum and incubated overnight in a water-jacketed, humidified incubator at 37° C. in an atmosphere supplemented with 5% carbon dioxide. The FGG siRNAs will be individually transfected using 0.2 μL Lipofectamine RNAiMax (Fisher, catalog #13778150) in 5 uL Opti-MEM (Thermo Fisher, catalog #31985070) per well. The positive control siRNA targeting FGG (ThermoFisher, catalog #4392420, Assay ID s5179) was included as a comparator. After incubation for 48 hours at 37° C., total RNA was harvested from each well using TaqMan® Fast Advanced Cells-to-CT™ Kit (ThermoFisher, catalog #A35374) according to the manufacturer's instructions. Preparation of cDNA was performed using Quanta qScript cDNA SuperMix (VWR, Catalog #95048-500) according to the manufacturer's instructions. The level of FGG mRNA from each well was measured in triplicate by biplex real-time qPCR on a QuantStudio 6 Pro instrument (Applied Biosystems) using TaqMan® Fast Advanced Master Mix (Fisher Scientific catalog #44-445-58), TaqMan Gene Expression Assay for human FGG (ThermoFisher, assay #Hs00241037_m1) and TaqMan Gene Expression Assay for human PPIA (ThermoFisher, assay #Hs99999904_m1). The relative FGG mRNA levels in each well was calculated using the delta-delta Ct method. All data were normalized to relative FGG mRNA levels in untreated Huh7 cells. Curve fit was accomplish using the [inhibitor] vs. response (three parameters) function in GraphPad Prism software. Results are shown in Table 17.

TABLE 17
IC50 Values of FGG siRNAs in Human Huh7 Cells

| | | | Relative FGG | |
|---|---|---|---|---|
| Untreated Cells | | | mRNA Levels | |
| siRNA | [siRNA] | | 1.00 | IC50 (nM) |
| Pos. Ctrl siRNA-1 | 30 | nM | 0.16 | 5.6 |
| | 10 | nM | 0.35 | |
| | 3 | nM | 0.41 | |
| | 1 | nM | 0.81 | |
| | 0.3 | nM | 0.83 | |
| | nM | | 0.64 | |
| | 0.03 | nM | | |
| ETD01663 | 30 | nM | 0.49 | 20.3 |
| | 10 | nM | 0.87 | |
| | 3 | nM | 1.05 | |
| | 1 | nM | 1.34 | |
| | 0.3 | nM | 1.42 | |
| | 0.1 | nM | 1.10 | |
| | 0.03 | nM | 1.32 | |
| ETD01665 | 30 | nM | 0.43 | 1.9 |
| | 10 | nM | 0.53 | |
| | 3 | nM | 0.77 | |
| | 1 | nM | 0.99 | |
| | 0.3 | nM | 1.16 | |
| | 0.1 | nM | 1.04 | |
| | 0.03 | nM | 1.49 | |
| ETD01666 | 30 | nM | 0.28 | 17.0 |
| | 10 | nM | 0.68 | |
| | 3 | nM | 1.02 | |
| | 1 | nM | 1.23 | |
| | 0.3 | nM | 1.29 | |
| | 0.1 | nM | 0.84 | |
| | 0.03 | nM | 1.54 | |
| ETD01670 | 30 | nM | 0.33 | 15.6 |
| | 10 | nM | 0.87 | |
| | 3 | nM | 1.11 | |
| | 1 | nM | 1.15 | |
| | 0.3 | nM | 1.41 | |
| | 0.1 | nM | 1.33 | |
| | 0.03 | nM | 1.60 | |
| ETD01686 | 30 | nM | 0.29 | 4.9 |
| | 10 | nM | 0.72 | |
| | 3 | nM | 1.07 | |
| | 1 | nM | 1.44 | |
| | 0.3 | nM | 1.72 | |
| | 0.1 | nM | 1.85 | |
| | 0.03 | nM | 1.59 | |
| ETD01694 | 30 | nM | 0.47 | 21.1 |
| | 10 | nM | 1.10 | |
| | 3 | nM | 1.31 | |
| | 1 | nM | 1.63 | |
| | 0.3 | nM | 1.88 | |
| | 0.1 | nM | 1.64 | |
| | 0.03 | nM | 1.61 | |
| ETD01697 | 30 | nM | 0.39 | 6.6 |
| | 10 | nM | 1.14 | |
| | 3 | nM | 1.18 | |
| | 1 | nM | 1.81 | |
| | 0.3 | nM | 2.07 | |
| | 0.1 | nM | 2.04 | |
| | 0.03 | nM | 1.89 | |
| ETD01701 | 30 | nM | 0.45 | 30.4 |
| | 10 | nM | 1.12 | |
| | 3 | nM | 1.62 | |
| | 1 | nM | 1.76 | |
| | 0.3 | nM | 1.88 | |
| | 0.1 | nM | 1.68 | |
| | 0.03 | nM | 1.88 | |
| ETD01703 | 30 | nM | 0.29 | 25.8 |
| | 10 | nM | 0.66 | |
| | 3 | nM | 0.87 | |
| | 1 | nM | 1.45 | |
| | 0.3 | nM | 1.00 | |
| | 0.1 | nM | 1.00 | |
| | 0.03 | nM | 1.09 | |
| ETD01710 | 30 | nM | 0.17 | 4.2 |
| | 10 | nM | 0.44 | |
| | 3 | nM | 0.70 | |
| | 1 | nM | 0.91 | |
| | 0.3 | nM | 1.09 | |
| | 0.1 | nM | 1.16 | |
| | 0.03 | nM | 1.16 | |

### Example 12. Optimization of siRNAs Targeting Human, Cynomolgous Monkey, Rat and Mouse FGG in Mice

Several siRNAs designed to be cross-reactive with human, cynomolgous monkey and mouse FGG mRNA were tested for activity in mice. The siRNAs were attached to the GalNAc ligand ETL17. The siRNA sequences are shown in Table 18, where “Nf” is a 2′ fluoro-modified nucleoside, “n” is a 2′ O-methyl modified nucleoside, and “s” is a phosphorothioate linkage.

Six to eight week old female mice (strain ICR, n=4) were given a subcutaneous injection on Day 0 of a single 20 ug or 60 ug dose of a GalNAc-conjugated siRNA or PBS as vehicle control.

Mice were euthanized on Day 14 after injection and a liver sample from each was collected and placed in RNAlater (ThermoFisher Catalog #AM7020) until processing. Total liver RNA was prepared by homogenizing the liver tissue in homogenization buffer (Maxwell RSC simplyRNA Tissue Kit) using a Percellys 24 tissue homogenizer (Bertin Instruments) set at 5000 rpm for two 10 second cycles. Total RNA from the lysate was purified on a Maxwell RSC 48 platform (Promega Corporation) according to the manufacturer's recommendations. Preparation of cDNA was performed using Quanta qScript cDNA SuperMix (VWR, Catalog #95048-500) according to the manufacturer's instructions. The relative levels of liver FGG mRNA were assessed by RT-qPCR in triplicate on a QuantStudio™ 6 Pro Real-Time PCR System using TaqMan assays for mouse FGG (ThermoFisher, assay #Mm00513575 ml) and the mouse housekeeping gene PPIA (ThermoFisher, assay #Mm02342430_g1) and PerfeCTa® qPCR FastMix®, Low ROX™ (VWR, Catalog #101419-222). Data were normalized to the mean FGG mRNA level in animals receiving PBS. Results are shown in Table 19.

On Day 0 (prior to dosing), 7 and Day 14 blood was collected into tubes with 0.2 mL sodium citrate for collection of plasma. Plasma samples were analyzed for fibrinogen levels by ELISA (Molecular Innovations Mouse Fibrinogen Antigen ELISA kit, Cat #MFBGNKT). Results are shown in Table 20.

On Day 14 blood was collected into tubes with 0.2 mL sodium citrate for collection of plasma. Plasma samples were analyzed for PT and APTT at IDEXX Laboratories (IDEXX Laboratories, Test #6005). Results are shown in Table 21.

TABLE 18A
Example siRNA Sequences

| | SEQ | | SEQ | |
|---|---|---|---|---|
| | ID | Sense Strand Sequence (5′-3′) | ID | |
| ETD# | NO | with GalNAc moiety | NO | Antisense Strand Sequence (5′-3′) |
| ETD01818 | 3650 | [ETL17]sgaugAfAfaGfAfuucgguaguasusu | 3686 | usAfscUfaCfcGfaAfuCfuUfuCfaUfcsusu |
| ETD01839 | 3652 | [ETL17]sgaugAfAfaGfAfuucgguaguasusu | 3688 | usAfscuaCfcGfaAfuCfuUfuCfaUfcsusu |
| ETD01840 | 3653 | [ETL17]sgaugAfAfaGfAfuucgguaguasusu | 3689 | usAfscUfaccGfaAfuCfuUfuCfaUfcsusu |
| ETD01841 | 3654 | [ETL17]sgaugAfAfaGfAfuucgguaguasusu | 3690 | usAfscUfacCfgaAfuCfuUfuCfaUfcsusu |

TABLE 18B
Example siRNA BASE Sequences

| | SEQ | Sense Strand Base | SEQ | Antisense Strand Base |
|---|---|---|---|---|
| siRNA | ID | Sequence (5′ to 3′), | ID | Sequence (5′ to 3′), |
| Name | NO: | without 3′ overhangs | NO: | without 3′ overhangs |
| ETD01818 | 3722 | UACCAAGGTGGCACUUACA | 3758 | UACUACCGAAUCUUUCAUC |
| ETD01839 | 3724 | GAUGAAAGAUUCGGUAGUA | 3760 | UACUACCGAAUCUUUCAUC |
| ETD01840 | 3725 | UGACAAGUUUUUCACAUCA | 3761 | UACUACCGAAUCUUUCAUC |
| ETD01841 | 3726 | GAAGAUUCAUUUGAUAAGA | 3762 | UACUACCGAAUCUUUCAUC |

TABLE 19
Relative FGG mRNA Levels in Livers of Mice

| | | | Dose | Mean FGG mRNA |
|---|---|---|---|---|
| Group | n | Treatment | (ug) | (Normalized to Group 1, Day 14) |
| 1 | 4 | PBS | 0 | 1.00375 |
| 2 | 4 | ETD01818 | 20 | 0.90475 |
| 3 | 4 | ETD01818 | 60 | 0.41 |
| 4 | 4 | ETD01839 | 20 | 0.5715 |
| 5 | 4 | ETD01839 | 60 | 0.317 |
| 6 | 4 | ETD01840 | 20 | 0.70675 |
| 7 | 4 | ETD01840 | 60 | 0.2125 |
| 8 | 4 | ETD01841 | 20 | 0.4445 |
| 9 | 4 | ETD01841 | 60 | 0.246 |

TABLE 20
Fibrinogen Levels in Plasma of Mice
treated with siRNAs targeting FGG

| | | | | Mean Fibrinogen | | |
|---|---|---|---|---|---|---|
| | | | Dose | (Normalized, Day 0) | | |
| Group | n | Treatment | (ug) | Day 0 | Day 7 | Day 14 |
| 1 | 4 | PBS | 0 | 1 | 1.211 | 0.732 |
| 2 | 4 | ETD01818 | 20 | 1 | 0.832 | 0.531 |
| 3 | 4 | ETD01818 | 60 | 1 | 0.478 | 0.245 |
| 4 | 4 | ETD01839 | 20 | 1 | 0.436 | 0.395 |
| 5 | 4 | ETD01839 | 60 | 1 | 0.186 | 0.142 |
| 6 | 4 | ETD01840 | 20 | 1 | 0.555 | 0.645 |
| 7 | 4 | ETD01840 | 60 | 1 | 0.433 | 0.402 |
| 8 | 4 | ETD01841 | 20 | 1 | 0.541 | 0.371 |
| 9 | 4 | ETD01841 | 60 | 1 | 0.157 | 0.180 |

TABLE 21
PT and APTT Times in Mice treated with siRNAs targeting FGG

| | | | Dose | Mean PT (sec) | Mean APPT (sec) |
|---|---|---|---|---|---|
| Group | n | Treatment | (ug) | Day 14 | Day 14 |
| 1 | 4 | PBS | 0 | 17.4 | 47.6 |
| 2 | 4 | ETD01818 | 20 | 18.375 | 43.875 |
| 3 | 4 | ETD01818 | 60 | 24.875 | 64.8 |
| 4 | 4 | ETD01839 | 20 | 18.225 | 46.075 |
| 5 | 4 | ETD01839 | 60 | 20.425 | 45.325 |
| 6 | 4 | ETD01840 | 20 | 15.65 | 28.3 |
| 7 | 4 | ETD01840 | 60 | 19.625 | 50.725 |
| 8 | 4 | ETD01841 | 20 | 21.375 | 41.875 |
| 9 | 4 | ETD01841 | 60 | 31.575 | 47.233 |

### Example 13. Optimization of siRNAs from Position 1218 Targeting Human, Cynomolgous Monkey, Rat and Mouse FGG in Mice

Several siRNAs designed to be cross-reactive with human, cynomolgous monkey and mouse FGG mRNA were tested for activity in mice. The siRNAs were attached to the GalNAc ligand ETL17. The siRNA sequences are shown in Table 22A, where “Nf” is a 2′ fluoro-modified nucleoside, “n” is a 2′ O-methyl modified nucleoside, and “s” is a phosphorothioate linkage.

Six to eight week old female mice (strain ICR, n=4) were given a subcutaneous injection on Day 0 of a single 60 ug dose of a GalNAc-conjugated siRNA or PBS as vehicle control.

Mice were euthanized on Day 10 after injection and a liver sample from each was collected and placed in RNAlater (ThermoFisher Catalog #AM7020) until processing. Total liver RNA was prepared by homogenizing the liver tissue in homogenization buffer (Maxwell RSC simplyRNA Tissue Kit) using a Percellys 24 tissue homogenizer (Bertin Instruments) set at 5000 rpm for two 10 second cycles. Total RNA from the lysate was purified on a Maxwell RSC 48 platform (Promega Corporation) according to the manufacturer's recommendations. Preparation of cDNA was performed using Quanta qScript cDNA SuperMix (VWR, Catalog #95048-500) according to the manufacturer's instructions. The relative levels of liver FGG mRNA were assessed by RT-qPCR in triplicate on a QuantStudio™ 6 Pro Real-Time PCR System using TaqMan assays for mouse FGG (ThermoFisher, assay #Mm00513575 ml) and the mouse housekeeping gene PPIA (ThermoFisher, assay #Mm02342430_g1) and PerfeCTa® qPCR FastMix®, Low ROX™ (VWR, Catalog #101419-222). Data were normalized to the mean FGG mRNA level in animals receiving PBS. Results are shown in Table 23.

On Day 0 (prior to dosing), 7 and Day 10 blood was collected into tubes with 0.2 mL sodium citrate for collection of plasma. Plasma samples were analyzed for fibrinogen levels by ELISA (Molecular Innovations Mouse Fibrinogen Antigen ELISA kit, Cat #MFBGNKT). Results are shown in Table 24.

On Day 14 blood was collected into tubes with 0.2 mL sodium citrate for collection of plasma. Plasma samples were analyzed for PT and APTT at IDEXX Laboratories (IDEXX Laboratories, Test #6005). Results are shown in Table 25.

TABLE 22A
Example siRNA Sequences

| | SEQ | | SEQ | |
|---|---|---|---|---|
| | ID | Sense Strand Sequence (5′-3′) | ID | |
| ETD# | NO | with GalNAc moiety | NO | Antisense Strand Sequence (5′-3′) |
| ETD01841 | 3654 | [ETL17]sgaugAfAfaGfAfuucgguaguasusu | 369 | usAfscUfacCfgaAfuCfuUfuCfaUfcsusu |
| ETD01852 | 3658 | [ETL17]sugacaagUfUfUfUfucacaucasusu | 3694 | usGfsaUfgUfgAfaAfaAfcUfuGfuCfasusu |
| ETD01921 | 3670 | [ETL17]sugacaagUfUfuUfucacaucasusu | 3706 | usGfsaUfgUfgAfaAfaAfcUfuGfuCfasusu |
| ETD01922 | 3671 | [ETL17]sugacaaguUfuUfuCfacaucasusu | 3707 | usGfsaUfgUfgAfaAfaAfcUfuGfuCfasusu |
| ETD01923 | 3672 | [ETL17]sugacaagUfUfUfUfucacaucasusu | 3708 | usGfsaugUfgAfaAfaAfcUfuGfuCfasusu |
| ETD01924 | 3673 | [ETL17]sugacaagUfUfUfUfucacaucasusu | 3709 | usGfsaUfgUfgaaAfaAfcUfuGfuCfasusu |
| ETD01925 | 3674 | [ETL17]sugacaagUfUfUfUfucacaucasusu | 3710 | usGfsaUfguGfAfaAfaAfcUfuGfuCfasusu |
| ETD01926 | 3675 | [ETL17]sugacaagUfUfUfUfucacaucasusu | 3711 | usGfsaUfguGfaaaaAfcUfuGfuCfasusu |
| ETD01927 | 3676 | [ETL17]sugacaagUfUfUfUfucacaucasusu | 3712 | usGfsaugUfgAfaaaAfcUfuGfuCfasusu |

TABLE 22B
Example siRNA BASE Sequences

| | | Sense Strand | | Antisense Strand |
|---|---|---|---|---|
| | | Base Sequence | | Base Sequence |
| | SEQ | (5′ to 3′), | SEQ | (5′ to 3′), |
| siRNA | ID | without 3′ | ID | without 3′ |
| Name | NO: | overhangs | NO: | overhangs |
| ETD01841 | 3726 | GAAGAUUCAUUU | 3762 | UACUACCGAAUCUUUC |
| | | GAUAAGA | | AUC |
| ETD01852 | 3730 | UACCAAGGTGGC | 3766 | UGAUGUGAAAAACUUG |
| | | ACUUACA | | UCA |
| ETD01921 | 3742 | GAUGAAAGAUUC | 3778 | UGAUGUGAAAAACUUG |
| | | GGUAGUA | | UCA |
| ETD01922 | 3743 | GAUGAAAGAUUC | 3779 | UGAUGUGAAAAACUUG |
| | | GGUAGUA | | UCA |
| ETD01923 | 3744 | GAUGAAAGAUUC | 3780 | UGAUGUGAAAAACUUG |
| | | GGUAGUA | | UCA |
| ETD01924 | 3745 | GAUGAAAGAUUC | 3781 | UGAUGUGAAAAACUUG |
| | | GGUAGUA | | UCA |
| ETD01925 | 3746 | CUCAAUGGAGUU | 3782 | UGAUGUGAAAAACUUG |
| | | UAUUACA | | UCA |
| ETD01926 | 3747 | UGACAAGUUUUU | 3783 | UGAUGUGAAAAACUUG |
| | | CACAUCA | | UCA |
| ETD01927 | 3748 | UCAAUGGAGUUU | 3784 | UGAUGUGAAAAACUUG |
| | | AUUACCA | | UCA |

TABLE 23
Relative FGG mRNA Levels in Livers of Mice

| | | | Dose | Mean FGG mRNA |
|---|---|---|---|---|
| Group | n | Treatment | (ug) | (Normalized to Group 1, Day 10) |
| 1 | 4 | PBS | 0 | 1.01025 |
| 2 | 4 | ETD01841 | 60 | 0.436 |
| 3 | 4 | ETD01852 | 60 | 0.91125 |
| 4 | 4 | ETD01921 | 60 | 0.788 |
| 5 | 4 | ETD01922 | 60 | 1.384 |
| 6 | 4 | ETD01923 | 60 | 0.98975 |
| 7 | 4 | ETD01924 | 60 | 0.85675 |
| 8 | 4 | ETD01925 | 60 | 0.7335 |
| 9 | 4 | ETD01926 | 60 | 0.57025 |
| 10 | 4 | ETD01927 | 60 | 0.73425 |

TABLE 24
Fibrinogen Levels in Plasma of Mice
treated with siRNAs targeting FGG

| | | | | Mean Fibrinogen | | |
|---|---|---|---|---|---|---|
| | | | Dose | (Normalized, Day 0) | | |
| Group | n | Treatment | (ug) | Day 0 | Day 7 | Day 10 |
| 1 | 4 | PBS | 0 | 1 | 1.38 | 1.32 |
| 2 | 4 | ETD01841 | 60 | 1 | 0.65 | 0.52 |
| 3 | 4 | ETD01852 | 60 | 1 | 1.17 | 0.68 |
| 4 | 4 | ETD01921 | 60 | 1 | 0.95 | 0.69 |
| 5 | 4 | ETD01922 | 60 | 1 | 0.93 | 0.93 |
| 6 | 4 | ETD01923 | 60 | 1 | 1.08 | 0.97 |
| 7 | 4 | ETD01924 | 60 | 1 | 0.85 | 0.68 |
| 8 | 4 | ETD01925 | 60 | 1 | 0.82 | 0.60 |
| 9 | 4 | ETD01926 | 60 | 1 | 0.50 | 0.31 |
| 10 | 4 | ETD01927 | 60 | 1 | 0.51 | 0.58 |

TABLE 25
PT and APTT Times in Mice treated with siRNAs targeting FGG

| | | | Dose | Mean PT (sec) | Mean APPT (sec) |
|---|---|---|---|---|---|
| Group | n | Treatment | (ug) | Day 10 | Day 10 |
| 1 | 4 | PBS | 0 | 14.15 | 34.97 |
| 2 | 4 | ETD01841 | 60 | 31.85 | 37.07 |
| 3 | 4 | ETD01852 | 60 | 15.55 | 31.97 |
| 4 | 4 | ETD01921 | 60 | 15.825 | 42.83 |
| 5 | 4 | ETD01922 | 60 | 16.05 | 32.23 |
| 6 | 4 | ETD01923 | 60 | 15.55 | 46.4 |
| 7 | 4 | ETD01924 | 60 | 15.075 | 35.13 |
| 8 | 4 | ETD01925 | 60 | 16.57 | 34.03 |
| 9 | 4 | ETD01926 | 60 | 16.775 | 35.78 |
| 10 | 4 | ETD01927 | 60 | 16.075 | 45.6 |

### Example 14. Testing Differentially Modified GalNAc siRNAs Targeting Human, Cynomolgous Monkey, Rat and Mouse FGG in Mice

Several siRNAs designed to be cross-reactive with human, cynomolgous monkey and mouse FGG mRNA were tested for activity in mice. The siRNAs were attached to the GalNAc ligand ETL1 or ETL17. The siRNA sequences are shown in Table 26A, where “Nf” is a 2′ fluoro-modified nucleoside, “n” is a 2′ O-methyl modified nucleoside, and “s” is a phosphorothioate linkage.

Six to eight week old female mice (strain ICR, n=4) were given a subcutaneous injection on Day 0 of a single 60 ug or 120 ug dose of a GalNAc-conjugated siRNA or PBS as vehicle control.

Mice were euthanized on Day 14 after injection and a liver sample from each was collected and placed in RNAlater (ThermoFisher Catalog #AM7020) until processing. Total liver RNA was prepared by homogenizing the liver tissue in homogenization buffer (Maxwell RSC simplyRNA Tissue Kit) using a Percellys 24 tissue homogenizer (Bertin Instruments) set at 5000 rpm for two 10 second cycles. Total RNA from the lysate was purified on a Maxwell RSC 48 platform (Promega Corporation) according to the manufacturer's recommendations. Preparation of cDNA was performed using Quanta qScript cDNA SuperMix (VWR, Catalog #95048-500) according to the manufacturer's instructions. The relative levels of liver FGG mRNA were assessed by RT-qPCR in triplicate on a QuantStudio™ 6 Pro Real-Time PCR System using TaqMan assays for mouse FGG (ThermoFisher, assay #Mm00513575 ml) and the mouse housekeeping gene PPIA (ThermoFisher, assay #Mm02342430_g1) and PerfeCTa® qPCR FastMix®, Low ROX™ (VWR, Catalog #101419-222). Data were normalized to the mean FGG mRNA level in animals receiving PBS. Results are shown in Table 27.

On Day 0 (prior to dosing), 7 and Day 14 blood was collected into tubes with 0.2 mL sodium citrate for collection of plasma. Plasma samples were analyzed for fibrinogen levels by ELISA (Molecular Innovations Mouse Fibrinogen Antigen ELISA kit, Cat #MFBGNKT). Results are shown in Table 28.

On Day 14 blood was collected into tubes with 0.2 mL sodium citrate for collection of plasma. Plasma samples were analyzed for PT and APTT at IDEXX Laboratories (IDEXX Laboratories, Test #6005). Results are shown in Table 29. Mice injected with ETD01811, ETD01818, and ETD01819 had an increase in PT and APPT times on Day 7 and 14 relative to mice receiving PBS.

On Days 0, 7, and 14 blood was collected into tubes with no anti-coagulant serum collected. Clinical chemistry parameters containing ALT, ALP, TBIL, and BUN were analyzed at IDEXX Laboratories (IDEXX Laboratories, Test #62849). Results are shown in Table 30.

TABLE 26A
Example siRNA Sequences

| | | Sense Strand | | Antisense |
|---|---|---|---|---|
| | SEQ | Sequence | SEQ | Strand |
| | ID | (5′-3′) with | ID | Sequence |
| ETD# | NO | GalNAc moiety | NO | (5′-3′) |
| ETD01811 | 3649 | [ETL1]sgaugAf | 3685 | usAfscUfaCfc |
| | | AfaGfAfuucggu | | GfaAfuCfuUfu |
| | | aguasusu | | CfaUfcsusu |
| ETD01818 | 3650 | [ETL17]sgaugA | 3686 | usAfscUfaCfc |
| | | fAfaGfAfuucgg | | GfaAfuCfuUfu |
| | | uaguasusu | | CfaUfcsusu |
| ETD01819 | 3651 | [ETL17]sgaagA | 3687 | usCfsuUfaUfc |
| | | fuucAfuuugaua | | AfaAfuGfaAfu |
| | | agasusu | | CfuUfcsusu |

TABLE 26B
Example siRNA Base Sequences

| | | Sense Strand | | Antisense Strand |
|---|---|---|---|---|
| | | Base Sequence | | Base Sequence |
| | SEQ | (5′ to 3′), | SEQ | (5′ to 3′), |
| siRNA | ID | without | ID | without |
| Name | NO: | 3′ overhangs | NO: | 3′ overhangs |
| ETD01811 | 3721 | UGACAAGUUUUUC | 3757 | UACUACCGAAUCUUUCAUC |
| | | ACAUCA | | |
| ETD01818 | 3722 | UACCAAGGTGGCA | 3758 | UACUACCGAAUCUUUCAUC |
| | | CUUACA | | |
| ETD01819 | 3723 | UGACAAGUUUUUC | 3759 | UCUUAUCAAAUGAAUCUUC |
| | | ACAUCA | | |

TABLE 27
Relative FGG mRNA Levels in Livers of Mice

| | | | Dose | Mean FGG mRNA |
|---|---|---|---|---|
| Group | n | Treatment | (ug) | (Normalized to Group 1, Day 14) |
| 1 | 4 | PBS | 0 | 1.00 |
| 2 | 4 | ETD01811 | 60 | 0.23 |
| 3 | 4 | ETD01811 | 120 | 0.14 |
| 4 | 4 | ETD01818 | 60 | 0.17 |
| 5 | 4 | ETD01818 | 120 | 0.14 |
| 6 | 4 | ETD01819 | 120 | 0.16 |

TABLE 28
Fibrinogen Levels in Plasma of Mice
treated with siRNAs targeting FGG

| | | | | Mean Fibrinogen | | |
|---|---|---|---|---|---|---|
| | | | Dose | (Normalized, Day 0) | | |
| Group | n | Treatment | (ug) | Day 0 | Day 7 | Day 14 |
| 1 | 4 | PBS | 0 | 1 | 1.03 | 0.74 |
| 2 | 4 | ETD01811 | 60 | 1 | 0.20 | 0.18 |
| 3 | 4 | ETD01811 | 120 | 1 | 0.13 | 0.06 |
| 4 | 4 | ETD01818 | 60 | 1 | 0.16 | 0.12 |
| 5 | 4 | ETD01818 | 120 | 1 | 0.11 | 0.08 |
| 6 | 4 | ETD01819 | 120 | 1 | 0.23 | 0.14 |

TABLE 29
PT and APTT Times in Mice treated with siRNAs targeting FGG

| | | | Dose | Mean PT (sec) | Mean APPT (sec) |
|---|---|---|---|---|---|
| Group | n | Treatment | (ug) | Day 14 | Day 14 |
| 1 | 4 | PBS | 0 | 14 | 39.45 |
| 2 | 4 | ETD01811 | 60 | 18.4 | 57.675 |
| 3 | 4 | ETD01811 | 120 | 46.4 | 83.425 |
| 4 | 4 | ETD01818 | 60 | 21.3 | 61.825 |
| 5 | 4 | ETD01818 | 120 | 27 | 59.325 |
| 6 | 4 | ETD01819 | 120 | 18.675 | 43.525 |

TABLE 30
Clinical Chemistry in Mice treated with siRNAs targeting FGG

| | | | | ALT | ALP | TBILI | BUN |
|---|---|---|---|---|---|---|---|
| | | | Dose | (U/L) | (U/L) | (mg/dL) | (mg/dL) |
| Group | n | Treatment | (ug) | Day 14 | Day 14 | Day 14 | Day 14 |
| 1 | 4 | PBS | 0 | 98.25 | 91.75 | 0.1 | 21.25 |
| 2 | 4 | ETD01818 | 20 | 53.75 | 106.75 | 0.1 | 22.5 |
| 3 | 4 | ETD01818 | 60 | 72 | 60 | 0.1 | 21.5 |
| 4 | 4 | ETD01839 | 20 | 39.25 | 75 | 0.125 | 24 |
| 5 | 4 | ETD01839 | 60 | 25 | 90.25 | 0.1 | 19 |
| 6 | 4 | ETD01840 | 20 | 25.75 | 90 | 0.15 | 22.75 |

### Example 15. Screening siRNAs from Position 352 Targeting Human, Cynomolgous Monkey, Rat and Mouse FGG in Mice

Several siRNAs designed to be cross-reactive with human, cynomolgous monkey and mouse FGG mRNA were tested for activity in mice. The siRNAs were attached to the GalNAc ligand ETL17. The siRNA sequences are shown in Table 31A, where “Nf” is a 2′ fluoro-modified nucleoside, “n” is a 2′ O-methyl modified nucleoside, “d” is a deoxynucleoside, and “s” is a phosphorothioate linkage.

Six to eight week old female mice (strain ICR, n=3) were given a subcutaneous injection on Day 0 of a single 40 ug dose of a GalNAc-conjugated siRNA or PBS as vehicle control. Mice were euthanized on Day 14 after injection and a liver sample from each was collected and placed in RNAlater (ThermoFisher Catalog #AM7020) until processing. Total liver RNA was prepared by homogenizing the liver tissue in homogenization buffer (Maxwell RSC simplyRNA Tissue Kit) using a Percellys 24 tissue homogenizer (Bertin Instruments) set at 5000 rpm for two 10 second cycles. Total RNA from the lysate was purified on a Maxwell RSC 48 platform (Promega Corporation) according to the manufacturer's recommendations. Preparation of cDNA was performed using Quanta qScript cDNA SuperMix (VWR, Catalog #95048-500) according to the manufacturer's instructions. The relative levels of liver FGG mRNA were assessed by RT-qPCR in triplicate on a QuantStudio™ 6 Pro Real-Time PCR System using TaqMan assays for mouse FGG (ThermoFisher, assay #Mm00513575 ml) and the mouse housekeeping gene PPIA (ThermoFisher, assay #Mm02342430_g1) and PerfeCTa® qPCR FastMix®, Low ROX™ (VWR, Catalog #101419-222). Data were normalized to the mean FGG mRNA level in animals receiving PBS. Results are shown in Table 32.

TABLE 31A
Example siRNA Sequences

| | | Sense Strand | | Antisense |
|---|---|---|---|---|
| | SEQ | Sequence | SEQ | Strand |
| | ID | (5′-3′) with | ID | Sequence |
| ETD# | NO | GalNAc moiety | NO | (5′-3′) |
| ETD01818 | 3650 | [ETL17]sgaugA | 3686 | usAfscUfaCfcGfa |
| | | fAfaGfAfuucgg | | AfuCfuUfuCfaUfc |
| | | uaguasusu | | susu |
| ETD01918 | 3667 | [ETL17]sgaugA | 3703 | usAfscuaCfcgaAf |
| | | fAfaGfAfuucgg | | uCfuUfuCfaUfcsu |
| | | uaguasusu | | su |
| ETD01919 | 3668 | [ETL17]sgaugA | 3704 | dTsAfscuaCfcGfa |
| | | fAfaGfAfuucgg | | AfuCfuUfuCfaUfc |
| | | uaguasusu | | susu |
| ETD01920 | 3669 | [ETL17]sgaugA | 3705 | UfsAfscuaCfcGfa |
| | | fAfaGfAfuucgg | | AfuCfuUfuCfaUfc |
| | | uaguasusu | | susu |
| ETD01870 | 3664 | [ETL17]saggug | 3700 | usAfscdTadCcGfa |
| | | gCfaCfUfuacuc | | AfudCudTudCadTc |
| | | aaaasusu | | susu |
| ETD01869 | 3665 | [ETL17]sgaugA | 3701 | usAfscdTadCcGfa |
| | | fAfaGfAfuucgg | | AfudCuUfudCaUfc |
| | | uaguasusu | | susu |

TABLE 31B
Example siRNA Base Sequences

| | | | | Antisense |
|---|---|---|---|---|
| | | Sense Strand | | Strand |
| | | Base Sequence | | Base Sequence |
| | SEQ | (5′ to 3′), | SEQ | (5′ to 3′), |
| siRNA | ID | without | ID | without |
| Name | NO: | 3′ overhangs | NO: | 3′ overhangs |
| ETD01818 | 3722 | UACCAAGGTGGCA | 3758 | UACUACCGAAUCUU |
| | | CUUACA | | UCAUC |
| ETD01918 | 3739 | GAUGAAAGAUUCG | 3775 | UACUACCGAAUCUU |
| | | GUAGUA | | UCAUC |
| ETD01919 | 3740 | GAUGAAAGAUUCG | 3776 | TACUACCGAAUCUU |
| | | GUAGUA | | UCAUC |
| ETD01920 | 3741 | GAUGAAAGAUUCG | 3777 | UACUACCGAAUCUU |
| | | GUAGUA | | UCAUC |
| ETD01870 | 3738 | GAAGAUUCAUUUG | 3774 | UACTACCGAAUCUT |
| | | AUAAGA | | UCATC |
| ETD01869 | 3737 | GAUGAAAGAUUCG | 3773 | UACTACCGAAUCUU |
| | | GUAGUA | | UCAUC |

TABLE 32
Relative FGG mRNA Levels in Livers of Mice

| | | | Dose | Mean FGG mRNA |
|---|---|---|---|---|
| Group | n | Treatment | (ug) | (Normalized to Group 1, Day 14) |
| 1 | 3 | PBS | 0 | 1.00 |
| 2 | 3 | ETD01818 | 40 | 0.66 |
| 3 | 3 | ETD01918 | 40 | 0.40 |
| 4 | 3 | ETD01919 | 40 | 1.06 |
| 5 | 3 | ETD01920 | 40 | 1.10 |
| 6 | 3 | ETD01870 | 40 | 1.71 |
| 7 | 3 | ETD01869 | 40 | 0.71 |

### Example 16. Screening siRNAs from siRCHv2 Targeting Human, Cynomolgous Monkey, Rat and Mouse FGG in Mice

Several siRNAs designed to be cross-reactive with human, cynomolgous monkey and mouse FGG mRNA were tested for activity in mice. The siRNAs were attached to the GalNAc ligand ETL1 or ETL17. The siRNA sequences are shown in Table 33A, where “Nf” is a 2′ fluoro-modified nucleoside, “n” is a 2′ O-methyl modified nucleoside, “d” is a deoxynucleoside, and “s” is a phosphorothioate linkage.

Six to eight week old female mice (strain ICR, n=3) were given a subcutaneous injection on Day 0 of a single 60 ug dose of a GalNAc-conjugated siRNA or PBS as vehicle control.

Mice were euthanized on Day 10 after injection and a liver sample from each was collected and placed in RNAlater (ThermoFisher Catalog #AM7020) until processing. Total liver RNA was prepared by homogenizing the liver tissue in homogenization buffer (Maxwell RSC simplyRNA Tissue Kit) using a Percellys 24 tissue homogenizer (Bertin Instruments) set at 5000 rpm for two 10 second cycles. Total RNA from the lysate was purified on a Maxwell RSC 48 platform (Promega Corporation) according to the manufacturer's recommendations. Preparation of cDNA was performed using Quanta qScript cDNA SuperMix (VWR, Catalog #95048-500) according to the manufacturer's instructions. The relative levels of liver FGG mRNA were assessed by RT-qPCR in triplicate on a QuantStudio™ 6 Pro Real-Time PCR System using TaqMan assays for mouse FGG (ThermoFisher, assay #Mm00513575 ml) and the mouse housekeeping gene PPIA (ThermoFisher, assay #Mm02342430_g1) and PerfeCTa® qPCR FastMix®, Low ROX™ (VWR, Catalog #101419-222). Data were normalized to the mean FGG mRNA level in animals receiving PBS. Results are shown in Table 34.

On Day 0 (prior to dosing), Day 10 blood was collected into tubes with 0.2 mL sodium citrate for collection of plasma. Plasma samples were analyzed for fibrinogen levels by ELISA (Molecular Innovations Mouse Fibrinogen Antigen ELISA kit, Cat #MFBGNKT). Results are shown in Table 35.

On Day 10 blood was collected into tubes with 0.2 mL sodium citrate for collection of plasma. Plasma samples were analyzed for PT and APTT at IDEXX Laboratories (IDEXX Laboratories, Test #6005). Results are shown in Table 36.

TABLE 33A
Example siRNA Sequences

| | | Sense Strand | | Antisense |
|---|---|---|---|---|
| | SEQ | Sequence | SEQ | Strand |
| | ID | (5′-3′) with | ID | Sequence |
| ETD# | NO | GalNAc moiety | NO | (5′-3′) |
| ETD01818 | 3650 | [ETL17\|sgaugA | 3686 | usAfscUfaCfc |
| | | fAfaGfAfuucgg | | GfaAfuCfuUfu |
| | | uaguasusu | | CfaUfcsusu |
| ETD01808 | 3647 | [ETL1]scucaAf | 3683 | usGfsuAfaUfa |
| | | uGfGfAfGfuuua | | AfaCfuCfcAfu |
| | | uuacasusu | | UfgAfgsusu |
| ETD01809 | 3648 | [ETL1]sucaauG | 3684 | usGfsgUfaAfu |
| | | fGfAfGfuuuauu | | AfaAfcUfcCfa |
| | | accasusu | | UfuGfasusu |
| ETD01849 | 3655 | [ETL17]sagaca | 3691 | usGfsaCfcAfa |
| | | UfCfaUfgaguug | | CfuCfaUfgAfu |
| | | gucasusu | | GfuCfususu |
| ETD01851 | 3656 | [ETL17]sagacu | 3692 | usGfsuUfcUfg |
| | | GfGfAfAfuGfgc | | CfcAfuUfcCfa |
| | | agaacasusu | | GfuCfususu |
| ETD01852 | 3657 | [ETL17]sugaca | 3693 | usGfsaUfgUfg |
| | | agUfUfUfUfuca | | AfaAfaAfcUfu |
| | | caucasusu | | GfuCfasusu |
| ETD01853 | 3658 | [ETL17]sugaca | 3694 | usGfsgAfuGfu |
| | | agUfUfUfUfuca | | GfaAfaAfaCfu |
| | | caucasusu | | UfgUfcsusu |
| ETD01855 | 3659 | [ETL17]sgacaa | 3695 | usCfsaCfcUfu |
| | | gUfUfUfUfUfca | | GfgUfaAfuAfa |
| | | cauccasusu | | AfcUfcsusu |
| ETD01856 | 3660 | [ETL17]sgaguU | 3696 | usGfsuAfaGfu |
| | | fUfaUfUfaCfca | | GfcCfaCfcUfu |
| | | aggugasusu | | GfgUfasusu |
| ETD01857 | 3661 | [ETL17]suaccA | 3697 | usAfsgUfaAfg |
| | | faGfGfdTggcac | | UfgCfcAfcCfu |
| | | uuacasusu | | UfgGfususu |
| ETD01858 | 3662 | [ETL17]saccaA | 3698 | usUfsuGfaGfu |
| | | fGfguGfGfcacu | | AfaGfuGfcCfa |
| | | uacuasusu | | CfcUfususu |
| ETD01859 | 3663 | [ETL17]saaggu | 3699 | usUfsuUfgAfg |
| | | GfGfcAfcuuacu | | UfaAfgUfgCfc |
| | | caaasusu | | AfcCfususu |

TABLE 33B
Example siRNA Base Sequences

| | | | | Antisense |
|---|---|---|---|---|
| | | Sense Strand | | Strand Base |
| | | Base Sequence | | Sequence |
| | SEQ | (5′ to 3′), | SEQ | (5′ to 3′), |
| siRNA | ID | without 3′ | ID | without 3′ |
| Name | NO: | overhangs | NO: | overhangs |
| ETD01818 | 3722 | UACCAAGGTGGCA | 3758 | UACUACCGAAUCU |
| | | CUUACA | | UUCAUC |
| ETD01808 | 3719 | UGACAAGUUUUUC | 3755 | UGUAAUAAACUCC |
| | | ACAUCA | | AUUGAG |
| ETD01809 | 3720 | UGACAAGUUUUUC | 3756 | UGGUAAUAAACUC |
| | | ACAUCA | | CAUUGA |
| ETD01849 | 3727 | GAUGAAAGAUUCG | 3763 | UGACCAACUCAUG |
| | | GUAGUA | | AUGUCU |
| ETD01851 | 3728 | AGACAUCAUGAGU | 3764 | UGUUCUGCCAUUC |
| | | UGGUCA | | CAGUCU |
| ETD01852 | 3729 | GACAAGUUUUUCA | 3765 | UGAUGUGAAAAAC |
| | | CAUCCA | | UUGUCA |
| ETD01853 | 3730 | UACCAAGGTGGCA | 3766 | UGGAUGUGAAAAA |
| | | CUUACA | | CUUGUC |
| ETD01855 | 3731 | ACCAAGGUGGCAC | 3767 | UCACCUUGGUAAU |
| | | UUACUA | | AAACUC |
| ETD01856 | 3732 | UGACAAGUUUUUC | 3768 | UGUAAGUGCCACC |
| | | ACAUCA | | UUGGUA |
| ETD01857 | 3733 | GAUGAAAGAUUCG | 3769 | UAGUAAGUGCCAC |
| | | GUAGUA | | CUUGGU |
| ETD01858 | 3734 | GAUGAAAGAUUCG | 3770 | UUUGAGUAAGUGC |
| | | GUAGUA | | CACCUU |
| ETD01859 | 3735 | UGACAAGUUUUUC | 3771 | UUUUGAGUAAGUG |
| | | ACAUCA | | CCACCU |

TABLE 34
Relative FGG mRNA Levels in Livers of Mice

| | | | Dose | Mean FGG mRNA |
|---|---|---|---|---|
| Group | n | Treatment | (ug) | (Normalized to Group 1, Day 10) |
| 1 | 3 | PBS | 0 | 1.00 |
| 2 | 3 | ETD01818 | 60 | 0.22 |
| 3 | 3 | ETD01808 | 60 | 1.12 |
| 4 | 3 | ETD01809 | 60 | 1.00 |
| 5 | 3 | ETD01849 | 60 | 0.64 |
| 6 | 3 | ETD01851 | 60 | 0.53 |
| 7 | 3 | ETD01852 | 60 | 0.33 |
| 8 | 3 | ETD01853 | 60 | 0.69 |
| 9 | 3 | ETD01855 | 60 | 0.96 |
| 10 | 3 | ETD01856 | 60 | 0.77 |
| 11 | 3 | ETD01857 | 60 | 0.71 |
| 12 | 3 | ETD01858 | 60 | 0.91 |
| 13 | 3 | ETD01859 | 60 | 1.00 |

TABLE 35
Fibrinogen Levels in Plasma of Mice
treated with siRNAs targeting FGG

| | |
|---|---|
| | Mean Fibrinogen |
| Dose | (Normalized, Day 0) |

| Group | n | Treatment | (ug) | Day 0 | Day 10 |
|---|---|---|---|---|---|
| 1 | 3 | PBS | 0 | 1.00 | 0.96 |
| 2 | 3 | ETD01818 | 60 | 1.00 | 0.34 |
| 3 | 3 | ETD01808 | 60 | 1.00 | 0.38 |
| 4 | 3 | ETD01809 | 60 | 1.00 | 0.53 |
| 5 | 3 | ETD01849 | 60 | 1.00 | 0.42 |
| 6 | 3 | ETD01851 | 60 | 1.00 | 0.80 |
| 7 | 3 | ETD01852 | 60 | 1.00 | 0.96 |
| 8 | 3 | ETD01853 | 60 | 1.00 | 0.34 |
| 9 | 3 | ETD01855 | 60 | 1.00 | 0.38 |
| 10 | 3 | ETD01856 | 60 | 1.00 | 0.53 |
| 11 | 3 | ETD01857 | 60 | 1.00 | 0.42 |
| 12 | 3 | ETD01858 | 60 | 1.00 | 0.80 |
| 13 | 3 | ETD01859 | 60 | 1.00 | 0.96 |

TABLE 36
PT and APTT Times in Mice treated with siRNAs targeting FGG

| | | | | Mean PT | Mean APPT |
|---|---|---|---|---|---|
| | | | Dose | (sec) | (sec) |
| Group | n | Treatment | (ug) | Day 14 | Day 14 |
| 1 | 3 | PBS | 0 | 16.17 | 35.10 |
| 2 | 3 | ETD01818 | 60 | 18.40 | 32.33 |
| 3 | 3 | ETD01808 | 60 | 17.30 | 47.60 |
| 4 | 3 | ETD01809 | 60 | 17.83 | 49.57 |
| 5 | 3 | ETD01849 | 60 | 21.27 | 44.50 |
| 6 | 3 | ETD01851 | 60 | 43.73 | 18.97 |
| 7 | 3 | ETD01852 | 60 | 48.83 | 18.10 |
| 8 | 3 | ETD01853 | 60 | 34.15 | 52.45 |
| 9 | 3 | ETD01855 | 60 | 18.20 | 45.50 |
| 10 | 3 | ETD01856 | 60 | 16.00 | 43.93 |
| 11 | 3 | ETD01857 | 60 | 15.90 | 48.67 |
| 12 | 3 | ETD01858 | 60 | 24.60 | 55.85 |
| 13 | 3 | ETD01859 | 60 | 17.43 | 39.13 |

### Example 17. Screening of siRNAs from Positions 352 and 1218 Targeting Human FGG mRNA in Mice Transfected with AAV8-TBG-h-FGG

Several siRNAs designed to be cross-reactive with human, cynomolgus monkey, rat and mouse FGG mRNA were tested for activity in mice following transfection with an adeno-associated viral vector. The siRNAs were attached to the GalNAc ligand ETL17. The siRNA sequences are shown in Table 37A, where “Nf” is a 2′ fluoro-modified nucleoside, “n” is a 2′ O-methyl modified nucleoside, “d” is a deoxynucleoside, and “s” is a phosphorothioate linkage.

Six to eight week old female mice (C57Bl/6) were injected with 10 μL of a recombinant adeno-associated virus 8 (AAV8) vector (2.1×10E13 genome copies/mL) by the retroorbital route on Day −14. The recombinant AAV8 contains the open reading frame and a portion of the 5′ and 3′UTRs of the human FGG sequence (ENST00000404648) under the control of the human thyroxine binding globulin promoter in an AAV2 backbone packaged in AAV8 capsid (AAV8-TBG-h-FGG). On Day 0, infected mice (n=3) were given a subcutaneous injection of a single 60 ug dose of a GalNAc-conjugated siRNA or PBS as vehicle control.

Mice were euthanized on Day 14 after subcutaneous injection and a liver sample from each was collected and placed in RNAlater (ThermoFisher Catalog #AM7020) until processing. Total liver RNA was prepared by homogenizing the liver tissue in homogenization buffer (Maxwell RSC simplyRNA Tissue Kit) using a Percellys 24 tissue homogenizer (Bertin Instruments) set at 5000 rpm for two 10 second cycles. Total RNA from the lysate was purified on a Maxwell RSC 48 platform (Promega Corporation) according to the manufacturer's recommendations. Preparation of cDNA was performed using Quanta qScript cDNA SuperMix (VWR, Catalog #95048-500) according to the manufacturer's instructions. The relative levels of liver MTRES1 mRNA were assessed by RT-qPCR in triplicate on a QuantStudio™ 6 Pro Real-Time PCR System using TaqMan assays for human FGG (ThermoFisher, assay #Hs00241038 ml), or mouse FGG (ThermoFisher, assay #Mm00513575_m1) and the mouse housekeeping gene PPIA (ThermoFisher, assay #Mm02342430_g1) and PerfeCTa® qPCR FastMix®, Low ROX™ (VWR, Catalog #101419-222). Data were normalized to the mean FGG mRNA level in animals receiving PBS. Mice injected with ETD01592, ETD01594, ETD01745, ETD01747, ETD01748, and ETD01750 had substantial reductions in mean liver mouse FGG mRNA on Day 14 relative to mice receiving PBS. Results are shown in Table 38. Mice injected with ETD01592, ETD01594, ETD01745, ETD01747, ETD01748, and ETD01750 had substantial reductions in mean liver human FGG mRNA on Day 14 relative to mice receiving PBS. Results are shown in Table 39.

On Day 0 (prior to dosing), 7 and Day 10 blood was collected into tubes with 0.2 mL sodium citrate for collection of plasma. Plasma samples were analyzed for fibrinogen levels by ELISA (Molecular Innovations Mouse Fibrinogen Antigen ELISA kit, Cat #MFBGNKT). Results are shown in Table 40.

On Day 14 blood was collected into tubes with 0.2 mL sodium citrate for collection of plasma. Plasma samples were analyzed for PT and APTT at IDEXX Laboratories (IDEXX Laboratories, Test #6005). Results are shown in Table 41. On average mice injected with ETD01592, ETD01594, ETD01745, ETD01747, ETD01748, and ETD01750 had no change in PT and APPT times on Day 14 relative to mice receiving PBS.

TABLE 37A
Example siRNA Sequences

| | | Sense Strand | | Antisense |
|---|---|---|---|---|
| | SEQ | Sequence | SEQ | Strand |
| | ID | (5′-3′) with | ID | Sequence |
| ETD# | NO | GalNAc moiety | NO | (5′-3′) |
| ETD01592 | 3641 | [ETL1]gsasugA | 3677 | usAfscUfaCfc |
| | | fAfaGfAfuucgg | | GfaAfuCfuUfu |
| | | uaguasusu | | CfaUfcsusu |
| ETD01594 | 3642 | [ETL1]gsasagA | 3678 | usCfsuUfaUfc |
| | | fuucAfuuugaua | | AfaAfuGfaAfu |
| | | agasusu | | CfuUfcsusu |
| ETD01745 | 3643 | [ETL1]sgaugAf | 3679 | usAfscUfaCfc |
| | | AfaGfAfuucgGf | | GfaAfuCfuUfu |
| | | uaGfuasusu | | CfaUfcsusu |
| ETD01747 | 3644 | [ETL1]sgaugAf | 3680 | usAfscUfaCfc |
| | | AfaGfdAuUfCfg | | GfaAfuCfuUfu |
| | | gUfaguasusu | | CfaUfcsusu |
| ETD01748 | 3645 | [ETL1]sgaAfGf | 3681 | usCfsuUfaUfc |
| | | auucAfuuugAfu | | AfaAfuGfaAfu |
| | | AfAfgasusu | | CfuUfcsusu |
| ETD01750 | 3646 | [ETL1]sgaAfgA | 3682 | usCfsuUfaUfc |
| | | fuUfCfdAuUfUf | | AfaAfuGfaAfu |
| | | gaUfaagasusu | | CfuUfcsusu |

TABLE 37B
Example siRNA Base Sequences

| | | Sense | | Antisense |
|---|---|---|---|---|
| | | Strand | | Strand |
| | | Base | | Base |
| | | Sequence | | Sequence |
| | SEQ | (5′ to 3′), | SEQ | (5′ to 3′), |
| siRNA | ID | without 3′ | ID | without 3′ |
| Name | NO: | overhangs | NO: | overhangs |
| ETD01592 | 3713 | GAUGAAAGAUUC | 3749 | UACUACCGAAUCU |
| | | GGUAGUA | | UUCAUC |
| ETD01594 | 3714 | GAUGAAAGAUUC | 3750 | UCUUAUCAAAUGA |
| | | GGUAGUA | | AUCUUC |
| ETD01745 | 3715 | GAUGAAAGAUUC | 3751 | UACUACCGAAUCU |
| | | GGUAGUA | | UUCAUC |
| ETD01747 | 3716 | GAUGAAAGAUUC | 3752 | UACUACCGAAUCU |
| | | GGUAGUA | | UUCAUC |
| ETD01748 | 3717 | GAUGAAAGAUUC | 3753 | UCUUAUCAAAUGA |
| | | GGUAGUA | | AUCUUC |
| ETD01750 | 3718 | UGACAAGUUUUU | 3754 | UCUUAUCAAAUGA |
| | | CACAUCA | | AUCUUC |

TABLE 38
Relative mouse FG mRNA Levels in Livers of Mice

| | | | Dose | Mean mouse MTRES1 mRNA |
|---|---|---|---|---|
| Group | n | Treatment | (ug) | (Normalized to Group 1, Day 14) |
| 1 | 3 | PBS | 0 | 1.00 |
| 2 | 3 | ETD01592 | 60 | 0.23 |
| 3 | 3 | ETD01594 | 60 | 0.35 |
| 4 | 3 | ETD01745 | 60 | 0.31 |
| 5 | 3 | ETD01747 | 60 | 0.27 |
| 6 | 3 | ETD01748 | 60 | 0.42 |
| 7 | 3 | ETD01750 | 60 | 0.49 |

TABLE 39
Relative human FGG mRNA Levels in Livers of Mice

| | | | Dose | Mean human MTRES1 mRNA |
|---|---|---|---|---|
| Group | n | Treatment | (ug) | (Normalized to Group 6, Day 14) |
| 1 | 3 | PBS | 0 | 1.00 |
| 2 | 3 | ETD01592 | 60 | 0.22 |
| 3 | 3 | ETD01594 | 60 | 0.43 |
| 4 | 3 | ETD01745 | 60 | 0.49 |
| 5 | 3 | ETD01747 | 60 | 0.24 |
| 6 | 3 | ETD01748 | 60 | 0.42 |
| 7 | 3 | ETD01750 | 60 | 0.51 |

TABLE 40
Fibrinogen Levels in Plasma of Mice
treated with siRNAs targeting FGG

| | |
|---|---|
| | Mean Fibrinogen |
| Dose | (Normalized, Day 0) |

| Group | n | Treatment | (ug) | Day 0 | Day 14 |
|---|---|---|---|---|---|
| 1 | 3 | PBS | 0 | 1 | 0.445 |
| 2 | 3 | ETD01592 | 60 | 1 | 0.242 |
| 3 | 3 | ETD01594 | 60 | 1 | 0.303 |
| 4 | 3 | ETD01745 | 60 | 1 | 0.216 |
| 5 | 3 | ETD01747 | 60 | 1 | 0.074 |
| 6 | 3 | ETD01748 | 60 | 1 | 1.209 |
| 7 | 3 | ETD01750 | 60 | 1 | 0.468 |

TABLE 41
PT and APTT Times in Mice treated with siRNAs targeting FGG

| | | | | Mean PT | Mean APPT |
|---|---|---|---|---|---|
| | | | Dose | (sec) | (sec) |
| Group | n | Treatment | (ug) | Day 10 | Day 10 |
| 1 | 3 | PBS | 0 | 20.643 | 81.215 |
| 2 | 3 | ETD01592 | 60 | 17.058 | 35.874 |
| 3 | 3 | ETD01594 | 60 | 14.684 | 36.348 |
| 4 | 3 | ETD01745 | 60 | 15.639 | 33.771 |
| 5 | 3 | ETD01747 | 60 | 25.907 | 55.384 |
| 6 | 3 | ETD01748 | 60 | 15.052 | 34.784 |
| 7 | 3 | ETD01750 | 60 | 14.162 | 34.452 |

### Example 18. Screening of siRNAs Targeting Human FGG mRNA in Mice Transfected with AAV8-TBG-h-FGG

Several siRNAs designed to be cross-reactive with human, cynomolgus monkey, rat and mouse FGG mRNA were tested for activity in mice following transfection with an adeno-associated viral vector. The siRNAs were attached to the GalNAc ligand ETL17. The siRNA sequences are shown in Table 42A, where “Nf” is a 2′ fluoro-modified nucleoside, “n” is a 2′ O-methyl modified nucleoside, “d” is a deoxynucleoside, and “s” is a phosphorothioate linkage.

Six to eight week old female mice (C57Bl/6) were injected with 10 μL of a recombinant adeno-associated virus 8 (AAV8) vector (2.1×10E13 genome copies/mL) by the retroorbital route on Day 14. The recombinant AAV8 contains the open reading frame and a portion of the 5′ and 3′UTRs of the human FGG sequence (ENST00000404648) under the control of the human thyroxine binding globulin promoter in an AAV2 backbone packaged in AAV8 capsid (AAV8-TBG-h-FGG). On Day 0, infected mice (n=4) were given a subcutaneous injection of a single 60 ug dose of a GalNAc-conjugated siRNA or PBS as vehicle control.

Mice were euthanized on Day 10 after subcutaneous injection and a liver sample from each was collected and placed in RNAlater (ThermoFisher Catalog #AM7020) until processing. Total liver RNA was prepared by homogenizing the liver tissue in homogenization buffer (Maxwell RSC simplyRNA Tissue Kit) using a Percellys 24 tissue homogenizer (Bertin Instruments) set at 5000 rpm for two 10 second cycles. Total RNA from the lysate was purified on a Maxwell RSC 48 platform (Promega Corporation) according to the manufacturer's recommendations. Preparation of cDNA was performed using Quanta qScript cDNA SuperMix (VWR, Catalog #95048-500) according to the manufacturer's instructions. The relative levels of liver MTRES1 mRNA were assessed by RT-qPCR in triplicate on a QuantStudio™ 6 Pro Real-Time PCR System using TaqMan assays for human FGG (ThermoFisher, assay #Hs00241038 ml), or mouse FGG (ThermoFisher, assay #Mm00513575_m1) and the mouse housekeeping gene PPIA (ThermoFisher, assay #Mm02342430_g1) and PerfeCTa® qPCR FastMix®, Low ROX™ (VWR, Catalog #101419-222). Data were normalized to the mean FGG mRNA level in animals receiving PBS. Mice injected with ETD01818, ETD01839, ETD01841, ETD01849, ETD01852, had greatest reductions in mean liver mouse FGG mRNA on Day 10 relative to mice receiving PBS. Results are shown in Table 43. Mice injected with ETD01818, ETD01839, ETD01841, ETD01849, and ETD01856 had greatest reductions in mean liver human FGG mRNA on Day 10 relative to mice receiving PBS. Results are shown in Table 44.

On Day 0 (prior to dosing), 7 and Day 10 blood was collected into tubes with 0.2 mL sodium citrate for collection of plasma. Plasma samples were analyzed for fibrinogen levels by ELISA (Molecular Innovations Mouse Fibrinogen Antigen ELISA kit, Cat #MFBGNKT). Results are shown in Table 45.

On Day 14 blood was collected into tubes with 0.2 mL sodium citrate for collection of plasma. Plasma samples were analyzed for PT and APTT at IDEXX Laboratories (IDEXX Laboratories, Test #6005). Results are shown in Table 46. Mice injected with ETD01818, ETD01839, ETD01840, and ETD01841 had an increase in PT and APPT times on Day 7 and 10 relative to mice receiving PBS.

TABLE 42A
Example siRNA Sequences

| | | Sense Strand | | Antisense |
|---|---|---|---|---|
| | SEQ | Sequence | SEQ | Strand |
| | ID | (5′-3′) with | ID | Sequence |
| ETD# | NO | GalNAc moiety | NO | (5′-3′) |
| ETD01818 | 3650 | [ETL17]sgaugA | 3686 | usAfscUfaCfc |
| | | fAfaGfAfuucgg | | GfaAfuCfuUfu |
| | | uaguasusu | | CfaUfcsusu |
| ETD01839 | 3652 | [ETL17]sgaugA | 3688 | usAfscuaCfcG |
| | | fAfaGfAfuucgg | | faAfuCfuUfuC |
| | | uaguasusu | | faUfcsusu |
| ETD01841 | 3654 | [ETL17]sgaugA | 3690 | usAfscUfacCf |
| | | fAfaGfAfuucgg | | gaAfuCfuUfuC |
| | | uaguasusu | | faUfcsusu |
| ETD01849 | 3655 | [ETL17]sagaca | 3691 | usGfsaCfcAfa |
| | | UfCfaUfgaguug | | CfuCfaUfgAfu |
| | | gucasusu | | GfuCfususu |
| ETD01851 | 3656 | [ETL17]sagacu | 3692 | usGfsuUfcUfg |
| | | GfGfAfAfuGfgc | | CfcAfuUfcCfa |
| | | agaacasusu | | GfuCfususu |
| ETD01852 | 3657 | [ETL17]sugaca | 3693 | usGfsaUfgUfg |
| | | agUfUfUfUfuca | | AfaAfaAfcUfu |
| | | caucasusu | | GfuCfasusu |
| ETD01853 | 3658 | [ETL17]sugaca | 3694 | usGfsgAfuGfu |
| | | agUfUfUfUfuca | | GfaAfaAfaCfu |
| | | caucasusu | | UfgUfcsusu |
| ETD01856 | 3659 | [ETL17]sgacaa | 3695 | usGfsuAfaGfu |
| | | gUfUfUfUfUfca | | GfcCfaCfcUfu |
| | | cauccasusu | | GfgUfasusu |
| ETD01857 | 3660 | [ETL17]sgaguU | 3696 | usAfsgUfaAfg |
| | | fUfaUfUfaCfca | | UfgCfcAfcCfu |
| | | aggugasusu | | UfgGfususu |

TABLE 42B
Example siRNA Base Sequences

| | | Sense | | Antisense |
|---|---|---|---|---|
| | | Strand | | Strand |
| | | Base | | Base |
| | | Sequence | | Sequence |
| | SEQ | (5′ to 3′), | SEQ | (5′ to 3′), |
| siRNA | ID | without 3′ | ID | without 3′ |
| Name | NO: | overhangs | NO: | overhangs |
| ETD01818 | 3722 | UACCAAGGTGG | 3758 | UACUACCGAAU |
| | | CACUUACA | | CUUUCAUC |
| ETD01839 | 3724 | GAUGAAAGAUU | 3760 | UACUACCGAAU |
| | | CGGUAGUA | | CUUUCAUC |
| ETD01841 | 3726 | GAAGAUUCAUU | 3762 | UACUACCGAAU |
| | | UGAUAAGA | | CUUUCAUC |
| ETD01849 | 3727 | GAUGAAAGAUU | 3763 | UGACCAACUCA |
| | | CGGUAGUA | | UGAUGUCU |
| ETD01851 | 3728 | AGACAUCAUGA | 3764 | UGUUCUGCCAU |
| | | GUUGGUCA | | UCCAGUCU |
| ETD01852 | 3729 | GACAAGUUUUU | 3765 | UGAUGUGAAAA |
| | | CACAUCCA | | ACUUGUCA |
| ETD01853 | 3730 | UACCAAGGTGG | 3766 | UGGAUGUGAAA |
| | | CACUUACA | | AACUUGUC |
| ETD01856 | 3731 | ACCAAGGUGGC | 3767 | UGUAAGUGCCA |
| | | ACUUACUA | | CCUUGGUA |
| ETD01857 | 3732 | UGACAAGUUUU | 3768 | UAGUAAGUGCC |
| | | UCACAUCA | | ACCUUGGU |

TABLE 43
Relative mouse FGG mRNA Levels in Livers of Mice

| | | | Dose | Mean mouse MTRES1 mRNA |
|---|---|---|---|---|
| Group | n | Treatment | (ug) | (Normalized to Group 1, Day 10) |
| 1 | 4 | PBS | 0 | 1.00 |
| 2 | 4 | ETD01818 | 60 | 0.21 |
| 3 | 4 | ETD01839 | 60 | 0.25 |
| 4 | 4 | ETD01841 | 60 | 0.21 |
| 5 | 4 | ETD01849 | 60 | 0.44 |
| 6 | 4 | ETD01851 | 60 | 0.76 |
| 7 | 4 | ETD01852 | 60 | 0.53 |
| 8 | 4 | ETD01853 | 60 | 0.73 |
| 9 | 4 | ETD01856 | 60 | 0.75 |
| 10 | 4 | ETD01857 | 60 | 0.83 |

TABLE 44
Relative human FGG mRNA Levels in Livers of Mice

| | | | Dose | Mean human MTRES1 mRNA |
|---|---|---|---|---|
| Group | n | Treatment | (ug) | (Normalized to Group 6, Day 10) |
| 1 | 4 | PBS | 0 | N/A |
| 2 | 4 | ETD01818 | 60 | 0.43 |
| 3 | 4 | ETD01839 | 60 | 0.52 |
| 4 | 4 | ETD01841 | 60 | 0.61 |
| 5 | 4 | ETD01849 | 60 | 0.26 |
| 6 | 4 | ETD01851 | 60 | 1.00 |
| 7 | 4 | ETD01852 | 60 | 0.76 |
| 8 | 4 | ETD01853 | 60 | 0.72 |
| 9 | 4 | ETD01856 | 60 | 0.43 |
| 10 | 4 | ETD01857 | 60 | 0.59 |

TABLE 45
Fibrinogen Levels in Plasma of Mice
treated with siRNAs targeting FGG

| | | | | Mean Fibrinogen | | |
|---|---|---|---|---|---|---|
| | | | Dose | (Normalized, Day 0) | | |
| Group | n | Treatment | (ug) | Day 0 | Day 4 | Day 10 |
| 1 | 4 | PBS | 0 | 1 | 0.843 | 0.630 |
| 2 | 4 | ETD01818 | 60 | 1 | 0.452 | 0.367 |
| 3 | 4 | ETD01839 | 60 | 1 | 0.516 | 0.178 |
| 4 | 4 | ETD01841 | 60 | 1 | 0.415 | 0.220 |
| 5 | 4 | ETD01849 | 60 | 1 | 0.724 | 0.619 |
| 6 | 4 | ETD01851 | 60 | 1 | 0.498 | 0.839 |
| 7 | 4 | ETD01852 | 60 | 1 | 0.671 | 0.565 |
| 8 | 4 | ETD01853 | 60 | 1 | 2.111 | 2.185 |
| 9 | 4 | ETD01856 | 60 | 1 | 1.436 | 1.343 |
| 10 | 4 | ETD01857 | 60 | 1 | 0.985 | 0.884 |

TABLE 46
PT and APTT Times in Mice treated with siRNAs targeting FGG

| | | | | Mean PT | Mean APPT |
|---|---|---|---|---|---|
| | | | Dose | (sec) | (sec) |
| Group | n | Treatment | (ug) | Day 10 | Day 10 |
| 1 | 4 | PBS | 0 | 27.8 | 53.825 |
| 2 | 4 | ETD01818 | 60 | 21.975 | 44.85 |
| 3 | 4 | ETD01839 | 60 | 38.525 | 79.275 |
| 4 | 4 | ETD01841 | 60 | 29.425 | 71.725 |
| 5 | 4 | ETD01849 | 60 | 20.6 | 64.875 |
| 6 | 4 | ETD01851 | 60 | 20.025 | 63.375 |
| 7 | 4 | ETD01852 | 60 | 23.325 | 82.3 |
| 8 | 4 | ETD01853 | 60 | 20.475 | 70.1 |
| 9 | 4 | ETD01856 | 60 | 21.5 | 65.2 |
| 10 | 4 | ETD01857 | 60 | 22.75 | 66.75 |

### Example 19. Screening of siRNAs Targeting Human FGG mRNA in Mice Transfected with AAV8-TBG-h-FGG

Several siRNAs designed to be cross-reactive with human, cynomolgus monkey, rat and mouse FGG mRNA were tested for activity in mice following transfection with an adeno-associated viral vector. The siRNAs were attached to the GalNAc ligand ETL17. The siRNA sequences are shown in Table 47A, where “Nf” is a 2′ fluoro-modified nucleoside, “n” is a 2′ O-methyl modified nucleoside, and “s” is a phosphorothioate linkage.

Six to eight week old female mice (C57Bl/6) were injected with 10 μL of a recombinant adeno-associated virus 8 (AAV8) vector (2.4×10E13 genome copies/mL) by the retroorbital route on Day −14. The recombinant AAV8 contains the open reading frame and a portion of the 5′ and 3′UTRs of the human FGG sequence (ENST00000404648) under the control of the human thyroxine binding globulin promoter in an AAV2 backbone packaged in AAV8 capsid (AAV8-TBG-h-FGG). On Day 0, infected mice (n=5) were given a subcutaneous injection of a single 60 μg or 100 ug dose of a GalNAc-conjugated siRNA or PBS as vehicle control.

Mice were euthanized on Day 10 after subcutaneous injection and a liver sample from each was collected and placed in RNAlater (ThermoFisher Catalog #AM7020) until processing. Total liver RNA was prepared by homogenizing the liver tissue in homogenization buffer (Maxwell RSC simplyRNA Tissue Kit) using a Percellys 24 tissue homogenizer (Bertin Instruments) set at 5000 rpm for two 10 second cycles. Total RNA from the lysate was purified on a Maxwell RSC 48 platform (Promega Corporation) according to the manufacturer's recommendations. Preparation of cDNA was performed using Quanta qScript cDNA SuperMix (VWR, Catalog #95048-500) according to the manufacturer's instructions. The relative levels of liver MTRESJ mRNA were assessed by RT-qPCR in triplicate on a QuantStudio™ 6 Pro Real-Time PCR System using TaqMan assays for human FGG (ThermoFisher, assay #Hs00241038 ml), or mouse FGG (ThermoFisher, assay #Mm00513575_m1) and the mouse housekeeping gene PPIA (ThermoFisher, assay #Mm02342430_g1) and PerfeCTa® qPCR FastMix®, Low ROX™ (VWR, Catalog #101419-222). Data were normalized to the mean FGG mRNA level in animals receiving PBS. Mice injected with ETD01818, ETD01839, and ETD01841 had substantial reductions in mean liver mouse FGG mRNA on Day 10 relative to mice receiving PBS at both 60 ug and 100 ug doses. Results are shown in Table 48. Mice injected with ETD01818, ETD01839, and ETD01841 had substantial reductions in mean liver human FGG mRNA on Day 10 relative to mice receiving PBS at both 60 ug and 100 ug doses. Results are shown in Table 49.

On Day 0 (prior to dosing), 7 and Day 10 blood was collected into tubes with 0.2 mL sodium citrate for collection of plasma. Plasma samples were analyzed for fibrinogen levels by ELISA (Molecular Innovations Mouse Fibrinogen Antigen ELISA kit, Cat #MFBGNKT). Results are shown in Table 50.

On Day 14 blood was collected into tubes with 0.2 mL sodium citrate for collection of plasma. Plasma samples were analyzed for PT and APTT at IDEXX Laboratories (IDEXX Laboratories, Test #6005). Results are shown in Table 51. Mice injected with ETD01818, ETD01839, and ETD01841 had dose dependent increase in PT and APPT times on Day 7 and 10 relative to mice receiving PBS.

TABLE 47A
Example siRNA Sequences

| | | Sense Strand | | Antisense |
|---|---|---|---|---|
| | SEQ | Sequence | SEQ | Strand |
| | ID | (5′-3′) with | ID | Sequence |
| ETD# | NO | GalNAc moiety | NO | (5′-3′) |
| ETD01818 | 3650 | [ETL17]sgaugAf | 3686 | usAfscUfaCfcG |
| | | AfaGfAfuucggua | | faAfuCfuUfuCf |
| | | guasusu | | aUfcsusu |
| ETD01839 | 3652 | [ETL17]sgaugAf | 3688 | usAfscuaCfcGf |
| | | AfaGfAfuucggua | | aAfuCfuUfuCfa |
| | | guasusu | | Ufcsusu |
| ETD01841 | 3654 | [ETL17]sgaugAf | 3690 | usAfscUfacCfg |
| | | AfaGfAfuucggua | | aAfuCfuUfuCfa |
| | | guasusu | | Ufcsusu |

TABLE 47B
Example siRNA BASE Sequences

| | | Sense | | Antisense |
|---|---|---|---|---|
| | | Strand | | Strand |
| | | Base | | Base |
| | | Sequence | | Sequence |
| | SEQ | (5′ to 3′), | SEQ | (5′ to 3′), |
| siRNA | ID | without 3′ | ID | without 3′ |
| Name | NO: | overhangs | NO: | overhangs |
| ETD01818 | 3722 | UACCAAGGTGG | 3758 | UACUACCGAAUC |
| | | CACUUACA | | UUUCAUC |
| ETD01839 | 3724 | GAUGAAAGAUU | 3760 | UACUACCGAAUC |
| | | CGGUAGUA | | UUUCAUC |
| ETD01841 | 3726 | GAAGAUUCAUU | 3762 | UACUACCGAAUC |
| | | UGAUAAGA | | UUUCAUC |

TABLE 48
Relative mouse FGG mRNA Levels in Livers of Mice

| | | | Dose | Mean mouse MTRES1 mRNA |
|---|---|---|---|---|
| Group | n | Treatment | (ug) | (Normalized to Group 1, Day 10) |
| 1 | 5 | PBS | 0 | 1.00 |
| 2 | 5 | ETD01818 | 60 | 0.36 |
| 3 | 5 | ETD01818 | 100 | 0.19 |
| 4 | 5 | ETD01839 | 60 | 0.24 |
| 5 | 5 | ETD01839 | 100 | 0.11 |
| 6 | 5 | ETD01841 | 60 | 0.17 |
| 7 | 5 | ETD01841 | 100 | 0.10 |

TABLE 49
Relative human FGG mRNA Levels in Livers of Mice

| | | | Dose | Mean human MTRES1 mRNA |
|---|---|---|---|---|
| Group | n | Treatment | (ug) | (Normalized to Group 6, Day 10) |
| 1 | 5 | PBS | 0 | 1.00 |
| 2 | 5 | ETD01818 | 60 | 0.56 |
| 3 | 5 | ETD01818 | 100 | 0.54 |
| 4 | 5 | ETD01839 | 60 | 0.49 |
| 5 | 5 | ETD01839 | 100 | 0.47 |
| 6 | 5 | ETD01841 | 60 | 0.71 |
| 7 | 5 | ETD01841 | 100 | 0.61 |

TABLE 50
Fibrinogen Levels in Plasma of Mice
treated with siRNAs targeting FGG

| | | | | Mean Fibrinogen | | |
|---|---|---|---|---|---|---|
| | | | Dose | (Normalized, Day 0) | | |
| Group | n | Treatment | (ug) | Day 0 | Day 4 | Day 10 |
| 1 | 5 | PBS | 0 | 1 | 0.901 | 0.914 |
| 2 | 5 | ETD01818 | 60 | 1 | 0.646 | 0.373 |
| 3 | 5 | ETD01818 | 100 | 1 | 0.520 | 0.205 |
| 4 | 5 | ETD01839 | 60 | 1 | 0.486 | 0.200 |
| 5 | 5 | ETD01839 | 100 | 1 | 0.370 | 0.090 |
| 6 | 5 | ETD01841 | 60 | 1 | 0.402 | 0.132 |
| 7 | 5 | ETD01841 | 100 | 1 | 0.436 | 0.100 |

TABLE 51
PT and APTT Times in Mice treated with siRNAs targeting FGG

| | | | | Mean PT | Mean APPT |
|---|---|---|---|---|---|
| | | | Dose | (sec) | (sec) |
| Group | n | Treatment | (ug) | Day 10 | Day 10 |
| 1 | 5 | PBS | 0 | 14.44 | 40.1 |
| 2 | 5 | ETD01818 | 60 | 16.9 | 39.08 |
| 3 | 5 | ETD01818 | 100 | 21.06 | 39.64 |
| 4 | 5 | ETD01839 | 60 | 20.64 | 50.52 |
| 5 | 5 | ETD01839 | 100 | 28.68 | 52.62 |
| 6 | 5 | ETD01841 | 60 | 19.32 | 36.18 |
| 7 | 5 | ETD01841 | 100 | 30.72 | 61.8 |

### Example 20. Determining the Activity of siRNAs Targeting FGG in Non-Human Primates

This study was conducted at Pharmalegacy Laboratories, Inc. on behalf of Empirico. Three groups (n=3/group) of 4-7 year old male cynomolgus monkeys (Zhaoqing Chuangyao Biotechnology Co., Ltd and Guangzhou Xianngguan Biotechnology Co., Ltd) were utilized for this study.

On Study Day 0, Group 1 cynomolgus monkeys were injected with 2 mg/kg ETD01839 (sense strand SEQ ID NO: 3652; antisense strand SEQ ID NO: 3688) at a concentration of 10 mg/mL, Group 2 cynomolgus monkeys were injected with 2 mg/kg ETD01841 (sense strand SEQ ID NO: 3654; antisense strand SEQ ID NO: 3690) at a concentration of 10 mg/mL, Group 3 cynomolgus monkeys were injected with 2 mg/kg ETD01926 (sense strand SEQ ID NO: 3675; antisense strand SEQ ID NO: 3711) at a concentration of 10 mg/mL. All animals had no abnormal clinical symptoms and well tolerated with single subcutaneous dose at 2 mg/kg of ETD01839, ETD01841 and ETD01926.

On Study Days −8, −2, 7, 14, 21 and Day 28 body weights were recorded. Results are shown in Table 52.

TABLE 52
Body Weights in Cynomolgus Monkeys treated
with siRNAs targeting FGG (kg)

| Treatment | Animal | | Body Weight (kg) | | | | | |
|---|---|---|---|---|---|---|---|---|
| group | No. | Gender | −8 | −2 | 7 | 14 | 21 | 28 |
| G1: | 101 | Male | 5.0 | 5.3 | 5.3 | 5.3 | 5.2 | 5.3 |
| ETD01839 | 102 | Male | 4.8 | 5.6 | 5.6 | 5.6 | 4.7 | 4.7 |
| | 103 | Male | 4.0 | 4.3 | 4.3 | 4.4 | 4.1 | 4.2 |
| G2: | 201 | Male | 5.2 | 5.7 | 5.7 | 5.6 | 5.2 | 5.3 |
| ETD01841 | 202 | Male | 4.5 | 5.1 | 5.1 | 5.2 | 4.2 | 4.3 |
| | 203 | Male | 5.1 | 5.4 | 5.4 | 5.3 | 5.4 | 5.3 |
| G3: | 301 | Male | 5.9 | 6.2 | 6.2 | 6.1 | 5.6 | 5.7 |
| ETD01926 | 302 | Male | 6.5 | 6.8 | 6.8 | 6.8 | 6.8 | 6.6 |
| | 303 | Male | 4.0 | 4.8 | 4.8 | 4.7 | 4.3 | 4.3 |

On Study Day −2 and Day 28, the animals were anesthetized with Zoletil (1.5-5.0 mg/kg, i.m.) and xylazine (0.5-2.0 mg/kg, i.m.) and 3-4 mg liver biopsy was collected. The biopsy was then placed in 10 v/v RNAlater in 20 seconds and stored for 24 hrs at 4° C., the RNAlater™ Stabilization Solution (Thermo Fisher, Catalog #AM7020) was then removed and the liver tissue was stored in freezer until they were shipped to Empirico. There were no abnormal clinical observations for all animals after liver biopsy collection on Day −2 or Day 28. The liver samples were processed in homogenization buffer (Maxwell RSC simplyRNA Tissue Kit) using Soft Tissue Homogenizing Kit CK14 (Bertin Instruments, catalog #P000933-LYSK0-A) in a Percellys 24 tissue homogenizer (Bertin Instruments) set at 5000 rpm for two 10 second cycles. Total RNA from the liver lysate was purified on a Maxwell RSC 48 platform (Promega Corporation) according to the manufacturer's recommendations. Preparation of cDNA was performed using Quanta qScript cDNA SuperMix (VWR, Catalog #95048-500) according to the manufacturer's instructions. The relative level of FGG mRNA in each Study Day 28 liver biopsy sample was assessed by RT-qPCR on a QuantStudio 6 Pro instrument (Applied Biosystems) using TaqMan assays for cyno FGG (ThermoFisher, assay #Mf02793821_m1) and the cyno housekeeping gene ACTB (ThermoFisher, assay #Mf04354341_g1), and then normalized to the mean value of the Study Day −2 pre-dose liver biopsy using the delta-delta Ct method. Animals treated with ETD01839, ETD01841 or ETD01926 showed decreased liver FGG mRNA levels on Study Day 28 compared to liver biopsies obtained from the same animals on Study Day −2. Results are shown in Table 53.

TABLE 53
Day 28 FGG mRNA liver levels in Cynomolgus Monkeys
treated with siRNAs targeting FGG

| | | | Relative Liver | | | |
|---|---|---|---|---|---|---|
| | | | FGG mRNA Level | | | |
| Treatment | Animal | | Day | Day −2 | Day | Day 28 |
| group | No. | Gender | −2 | Mean | 28 | Mean |
| G1: | 101 | Male | 1.961 | 1.00 | 0.533 | 0.65 |
| ETD01839 | 102 | Male | 0.322 | | 0.812 | |
| | 103 | Male | 1.586 | | 0.649 | |
| G2: | 201 | Male | 2.125 | 1.00 | 0.38 | 0.34 |
| ETD01841 | 202 | Male | 0.379 | | 0.383 | |
| | 203 | Male | 1.242 | | 0.274 | |
| G3: | 301 | Male | 1.62 | 1.00 | 0.265 | 0.23 |
| ETD01926 | 302 | Male | 3.462 | | 0.214 | |
| | 303 | Male | 0.178 | | 0.21 | |

On Study Days −2, −8, 7, 14, 21, and Day 28 blood was collected into tubes with 0.2 mL sodium citrate for collection of plasma. Plasma samples were analyzed for PT and APTT at Pharmalegacy Laboratories, Inc. and the remaining plasma samples were stored in a freezer until they were shipped to Empirico. Plasma sample were then transferred to IDEXX Laboratories and plasma fbrinogen levels were measured by the Clauss method (IDEXX Laboratories, Test #6308). Results are shown in Table 54-55. Animals treated with ETD01839, ETD01841 or ETD01926 showed a decrease in plasma fibrinogen starting on Study Day 7 though Study Day 28 when compared to Study Day −8 and Study Day −2, prior to treatment. Results are shown in Table 56.

TABLE 54
Prothrombin time in Cynomolgus
Monkeys treated with siRNAs targeting FGG

| Treatment | Animal | | PT (sec) | | | | | |
|---|---|---|---|---|---|---|---|---|
| group | No. | Gender | −8 | −2 | 7 | 14 | 21 | 28 |
| G1: | 101 | Male | 9.6 | 9.8 | 10.1 | 11.9 | 11.6 | 11.6 |
| ETD01839 | 102 | Male | 10.3 | 10.2 | 10.3 | 11.3 | 10.2 | 11.7 |
| | 103 | Male | 9.7 | 10.3 | 9.8 | 10.9 | 11 | 10.7 |
| G2: | 201 | Male | 9.3 | 9.9 | 10.5 | 12.3 | 12 | 12.4 |
| ETD01841 | 202 | Male | 9.2 | 9.9 | 10.4 | 11.9 | 12 | 11.8 |
| | 203 | Male | 9.7 | 9.5 | 9.3 | 9.9 | 6.6 | 10.2 |
| G3: | 301 | Male | 9.5 | 10 | 9.7 | 10.6 | 10.7 | 11.1 |
| ETD01926 | 302 | Male | 9.1 | 10 | 10.2 | 11.1 | 11.3 | 11.9 |
| | 303 | Male | 9.8 | 10.6 | 10.3 | 11.3 | 11.6 | 11.4 |

TABLE 55
Activated Partial Thromboplastin time in Cynomolgus
Monkeys treated with siRNAs targeting FGG

| Treatment | Animal | | APTT (sec) | | | | | |
|---|---|---|---|---|---|---|---|---|
| group | No. | Gender | −8 | −2 | 7 | 14 | 21 | 28 |
| G1: | 101 | Male | 18.7 | 20.5 | 19.6 | 19.2 | 19.2 | 17.8 |
| ETD01839 | 102 | Male | 19.4 | 20.2 | 19.7 | 20.1 | 19.8 | 17.9 |
| | 103 | Male | 18.7 | 20.8 | 19.5 | 19.4 | 20.1 | 18.6 |
| G2: | 201 | Male | 18.5 | 18.8 | 18.9 | 18.4 | 19.5 | 17.7 |
| ETD01841 | 202 | Male | 18.4 | 20.5 | 18.4 | 17.8 | 18.6 | 17.4 |
| | 203 | Male | 22.5 | 20.6 | 19.2 | 18.6 | 19.2 | 18.7 |
| G3: | 301 | Male | 19.3 | 20.6 | 19.4 | 20.1 | 19.7 | 18.5 |
| ETD01926 | 302 | Male | 15.5 | 18.2 | 16.8 | 16.9 | 16.6 | 15.5 |
| | 303 | Male | 21.7 | 22.4 | 22.4 | 23.4 | 21.2 | 20.6 |

TABLE 56
Plasma fibrinogen levels in Cynomolgus Monkeys
treated with siRNAs targeting FGG

| | | | Fibrinogen Plasma | | | | | |
|---|---|---|---|---|---|---|---|---|
| Treatment | Animal | | Level (Clauss method, mg/dL) | | | | | |
| group | No. | Gender | −8 | −2 | 7 | 14 | 21 | 28 |
| G1: | 101 | Male | N/A | 373 | 253 | 148 | 151 | 81 |
| ETD01839 | 102 | Male | 478 | 391 | 259 | 124 | 222 | 203 |
| | 103 | Male | 343 | 419 | 248 | 188 | 80 | 208 |
| G2: | 201 | Male | 277 | 316 | 180 | 117 | 155 | 120 |
| ETD01841 | 202 | Male | 581 | 272 | 169 | 126 | 148 | 125 |
| | 203 | Male | 674 | 519 | 286 | 295 | 82 | 318 |
| G3: | 301 | Male | 598 | 418 | 464 | 234 | 234 | 227 |
| ETD01926 | 302 | Male | 663 | 390 | 479 | 154 | 140 | 129 |
| | 303 | Male | 339 | 311 | 241 | 188 | 174 | 158 |

On Study Days −8, −2, 7, 14, 21, and Day 28 blood was collected into tubes with no anti-coagulant and serum collected. Clinical chemistry parameters containing ALT, AST, ALP, DBIL, TBIL, GLU, UREA, CREA, TP and CGT were analyzed at Pharmalegacy Laboratories, Inc.

Results are shown in Table 57-66.

TABLE 57
Clinical Chemistry ALT results of Cynomolgus Monkeys
treated with siRNAs targeting FGG

| Treatment | Animal | | ALT (U/L) | | | | | |
|---|---|---|---|---|---|---|---|---|
| group | No. | Gender | −8 | −2 | 7 | 14 | 21 | 28 |
| G1: | 101 | Male | 60 | 43 | 52 | 50 | 67 | 41 |
| ETD01839 | 102 | Male | 40 | 29 | 28 | 32 | 33 | 49 |
| | 103 | Male | 55 | 42 | 46 | 39 | 41 | 40 |
| G2: | 201 | Male | 68 | 63 | 59 | 51 | 59 | 35 |
| ETD01841 | 202 | Male | 159 | 88 | 81 | 55 | 52 | 48 |
| | 203 | Male | 55 | 39 | 46 | 39 | 37 | 64 |
| G3: | 301 | Male | 66 | 42 | 41 | 40 | 40 | 43 |
| ETD01926 | 302 | Male | 71 | 55 | 106 | 70 | 57 | 25 |
| | 303 | Male | 44 | 40 | 59 | 52 | 43 | 59 |

TABLE 58
Clinical Chemistry AST results of Cynomolgus
Monkeys treated with siRNAs targeting FGG

| Treatment | Animal | | AST (U/L) | | | | | |
|---|---|---|---|---|---|---|---|---|
| group | No. | Gender | −8 | −2 | 7 | 14 | 21 | 28 |
| G1: | 101 | Male | 61 | 48 | 48 | 55 | 99 | 43 |
| ETD01839 | 102 | Male | 32 | 35 | 35 | 33 | 41 | 41 |
| | 103 | Male | 50 | 62 | 61 | 53 | 69 | 43 |
| G2: | 201 | Male | 51 | 54 | 56 | 52 | 53 | 30 |
| ETD01841 | 202 | Male | 54 | 56 | 54 | 44 | 51 | 49 |
| | 203 | Male | 36 | 32 | 34 | 31 | 39 | 48 |
| G3: | 301 | Male | 44 | 51 | 43 | 46 | 43 | 56 |
| ETD01926 | 302 | Male | 38 | 62 | 50 | 46 | 44 | 30 |
| | 303 | Male | 32 | 33 | 41 | 37 | 38 | 61 |

TABLE 59
Clinical Chemistry ALP results of Cynomolgus
Monkeys treated with siRNAs targeting FGG

| Treatment | Animal | | ALP(U/L) | | | | | |
|---|---|---|---|---|---|---|---|---|
| group | No. | Gender | −8 | −2 | 7 | 14 | 21 | 28 |
| G1: | 101 | Male | 580 | 593 | 597 | 612 | 664 | 586 |
| ETD01839 | 102 | Male | 292 | 326 | 318 | 292 | 335 | 457 |
| | 103 | Male | 389 | 390 | 397 | 349 | 406 | 687 |
| G2: | 201 | Male | 590 | 667 | 591 | 572 | 610 | 289 |
| ETD01841 | 202 | Male | 509 | 523 | 533 | 553 | 535 | 619 |
| | 203 | Male | 263 | 288 | 308 | 266 | 289 | 618 |
| G3: | 301 | Male | 678 | 692 | 663 | 606 | 659 | 373 |
| ETD01926 | 302 | Male | 431 | 469 | 895 | 652 | 490 | 271 |
| | 303 | Male | 481 | 495 | 543 | 546 | 569 | 636 |

TABLE 60
Clinical Chemistry DBIL results of Cynomolgus
Monkeys treated with siRNAs targeting FGG

| Treatment | Animal | | DBIL (μmol/L) | | | | | |
|---|---|---|---|---|---|---|---|---|
| group | No. | Gender | −8 | −2 | 7 | 14 | 21 | 28 |
| G1: | 101 | Male | 0.4 | 0.4 | 0.7 | 0.7 | 1.1 | 0.6 |
| ETD01839 | 102 | Male | 0.4 | 0.4 | 0.7 | 0.7 | 0.8 | 0.5 |
| | 103 | Male | 0.9 | 0.5 | 0.7 | 0.7 | 0.7 | 0.7 |
| G2: | 201 | Male | 0.2 | 0.8 | 1.1 | 0.8 | 0.7 | 0.8 |
| ETD01841 | 202 | Male | 0.5 | 0.6 | 0.7 | 0.8 | 0.6 | 0.6 |
| | 203 | Male | 0.4 | 0.5 | 0.9 | 0.7 | 0.8 | 0.8 |
| G3: | 301 | Male | 0.6 | 0.7 | 0.7 | 0.7 | 0.8 | 0.4 |
| ETD01926 | 302 | Male | 0.5 | 0.8 | 1.1 | 0.5 | 0.9 | 0.7 |
| | 303 | Male | 0.4 | 0.5 | 1.2 | 1.3 | 1 | 0.8 |

TABLE 61
Clinical Chemistry TBIL results of Cynomolgus
Monkeys treated with siRNAs targeting FGG

| Treatment | Animal | | TBIL (μmol/L) | | | | | |
|---|---|---|---|---|---|---|---|---|
| group | No. | Gender | −8 | −2 | 7 | 14 | 21 | 28 |
| G1: | 101 | Male | 1 | 0.3 | 1.1 | 1.3 | 1.6 | 1.6 |
| ETD01839 | 102 | Male | 0.2 | 0 | 1 | 1.1 | 1.5 | 1.5 |
| | 103 | Male | 0.3 | 0.1 | 0.7 | 1.2 | 0.8 | 1.4 |
| G2: | 201 | Male | 0.8 | 1 | 1.6 | 1.5 | 1.3 | 1.4 |
| ETD01841 | 202 | Male | 1.2 | 0.7 | 0.9 | 1.2 | 0.8 | 0.9 |
| | 203 | Male | 0.1 | 0.5 | 1 | 0.8 | 1.1 | 2.1 |
| G3: | 301 | Male | 0.8 | 0.4 | 0.7 | 1.4 | 0.9 | 0.8 |
| ETD01926 | 302 | Male | 0.6 | 1.5 | 1.9 | 1.9 | 1.2 | 1.5 |
| | 303 | Male | 0.1 | 1.1 | 1.9 | 2.2 | 1.7 | 2.3 |

TABLE 62
Clinical Chemistry GLU results of Cynomolgus
Monkeys treated with siRNAs targeting FGG

| Treatment | Animal | | GLU (mmol/L) | | | | | |
|---|---|---|---|---|---|---|---|---|
| group | No. | Gender | −8 | −2 | 7 | 14 | 21 | 28 |
| G1: | 101 | Male | 5.8 | 3.1 | 5.7 | 5.2 | 2.3 | 5.5 |
| ETD01839 | 102 | Male | 5.1 | 3.4 | 4.3 | 4.3 | 2.7 | 3.8 |
| | 103 | Male | 4 | 2.6 | 3.8 | 3.1 | 2.9 | 3.1 |
| G2: | 201 | Male | 4.2 | 3.1 | 5.7 | 4.6 | 3.8 | 3.8 |
| ETD01841 | 202 | Male | 7.3 | 4.1 | 5.4 | 4.3 | 3.8 | 4.7 |
| | 203 | Male | 3.7 | 2.7 | 4.5 | 3.2 | 2.6 | 5.3 |
| G3: | 301 | Male | 5.5 | 2.8 | 2.8 | 3.2 | 3.3 | 3.2 |
| ETD01926 | 302 | Male | 4.5 | 2.5 | 4 | 3.2 | 3.3 | 5 |
| | 303 | Male | 5.1 | 3.6 | 4.1 | 3.8 | 3.3 | 4.1 |

TABLE 63
Clinical Chemistry UREA results of Cynomolgus
Monkeys treated with siRNAs targeting FGG

| Treatment | Animal | | UREA (mmol/L) | | | | | |
|---|---|---|---|---|---|---|---|---|
| group | No. | Gender | −8 | −2 | 7 | 14 | 21 | 28 |
| G1: | 101 | Male | 6.04 | 4.93 | 5.19 | 6.38 | 6.62 | 6.15 |
| ETD01839 | 102 | Male | 6.26 | 5.58 | 5.73 | 5.54 | 6.36 | 6.57 |
| | 103 | Male | 7.32 | 6.82 | 8.03 | 7.17 | 7.21 | 8.39 |
| G2: | 201 | Male | 6.12 | 7.14 | 6.39 | 6.6 | 6.33 | 7.84 |
| ETD01841 | 202 | Male | 5.87 | 5.48 | 6.46 | 5.87 | 6.2 | 5.75 |
| | 203 | Male | 8.72 | 7.07 | 8.37 | 8.59 | 9.2 | 6.22 |
| G3: | 301 | Male | 8.18 | 7.28 | 8.57 | 8.59 | 7.53 | 6.62 |
| ETD01926 | 302 | Male | 5.8 | 6.1 | 6.15 | 6.6 | 7.05 | 5.77 |
| | 303 | Male | 6.17 | 5.69 | 6.53 | 7.07 | 5.98 | 6.86 |

TABLE 64
Clinical Chemistry CREA results of Cynomolgus
Monkeys treated with siRNAs targeting FGG

| Treatment | Animal | | CREA (μmol/L) | | | | | |
|---|---|---|---|---|---|---|---|---|
| group | No. | Gender | −8 | −2 | 7 | 14 | 21 | 28 |
| G1: | 101 | Male | 56 | 52 | 58 | 57 | 59 | 65 |
| ETD01839 | 102 | Male | 63 | 62 | 66 | 67 | 62 | 92 |
| | 103 | Male | 69 | 68 | 69 | 67 | 67 | 79 |
| G2: | 201 | Male | 63 | 60 | 60 | 61 | 60 | 76 |
| ETD01841 | 202 | Male | 66 | 60 | 62 | 65 | 63 | 65 |
| | 203 | Male | 73 | 67 | 82 | 75 | 77 | 60 |
| G3: | 301 | Male | 72 | 71 | 83 | 77 | 70 | 66 |
| ETD01926 | 302 | Male | 93 | 89 | 87 | 87 | 86 | 66 |
| | 303 | Male | 61 | 56 | 63 | 60 | 58 | 57 |

TABLE 65
Clinical Chemistry TP results of Cynomolgus
Monkeys treated with siRNAs targeting FGG

| Treatment | Animal | | TP (g/L) | | | | | |
|---|---|---|---|---|---|---|---|---|
| group | No. | Gender | −8 | −2 | 7 | 14 | 21 | 28 |
| G1: | 101 | Male | 76.9 | 73.3 | 79 | 71 | 70.3 | 75.1 |
| ETD01839 | 102 | Male | 66.8 | 68.3 | 68.1 | 63.9 | 68.1 | 81.3 |
| | 103 | Male | 80.3 | 78.2 | 79.2 | 69.5 | 77.5 | 76.5 |
| G2: | 201 | Male | 74 | 78.3 | 75.6 | 67.8 | 73.3 | 73.8 |
| ETD01841 | 202 | Male | 77.9 | 80.5 | 82.1 | 80.1 | 75.2 | 84.7 |
| | 203 | Male | 72.3 | 73.4 | 78.9 | 69.8 | 70.4 | 74.8 |
| G3: | 301 | Male | 71.6 | 75.1 | 77.4 | 70.2 | 68.5 | 76.9 |
| ETD01926 | 302 | Male | 79.9 | 76.5 | 83.9 | 81.3 | 75 | 71 |
| | 303 | Male | 71.1 | 70.7 | 74.3 | 71.8 | 70.1 | 75 |

TABLE 66
Clinical Chemistry CGT results of Cynomolgus
Monkeys treated with siRNAs targeting FGG

| Treatment | Animal | | CGT (U/L) | | | | | |
|---|---|---|---|---|---|---|---|---|
| group | No. | Gender | −8 | −2 | 7 | 14 | 21 | 28 |
| G1: | 101 | Male | 81 | 77 | 81 | 81 | 80 | 70 |
| ETD01839 | 102 | Male | 83 | 81 | 97 | 90 | 100 | 88 |
| | 103 | Male | 111 | 111 | 104 | 97 | 109 | 118 |
| G2: | 201 | Male | 86 | 94 | 89 | 85 | 89 | 59 |
| ETD01841 | 202 | Male | 123 | 127 | 117 | 123 | 117 | 139 |
| | 203 | Male | 51 | 55 | 59 | 51 | 50 | 90 |
| G3: | 301 | Male | 109 | 115 | 107 | 103 | 106 | 107 |
| ETD01926 | 302 | Male | 60 | 56 | 135 | 112 | 93 | 94 |
| | 303 | Male | 59 | 61 | 65 | 64 | 65 | 79 |

### Example 21. Discovery Toxicity siRNAs Targeting Human, Cynomolgous Monkey, Rat and Mouse FGG in Mice

Several siRNAs designed to be cross-reactive with human, cynomolgous monkey and mouse FGG mRNA were tested for toxicity in mice. The siRNAs were attached to the GalNAc ligand ETL17. The siRNA sequences are shown in Table 66A, where Nf is a 2′ fluoro-modified nucleoside, n is a 2′ O-methyl modified nucleoside, and “s” is a phosphorothioate linkage.

Six to eight week old female mice (strain ICR, n=4) were given a subcutaneous injection on Day 0, 7, and Day 14 of a 200 ug dose of a GalNAc-conjugated siRNA or PBS as vehicle control.

Mice were euthanized on Day 14 after injection and a liver sample from each was collected and placed in RNAlater (ThermoFisher Catalog #AM7020) until processing. Total liver RNA was prepared by homogenizing the liver tissue in homogenization buffer (Maxwell RSC simplyRNA Tissue Kit) using a Percellys 24 tissue homogenizer (Bertin Instruments) set at 5000 rpm for two 10 second cycles. Total RNA from the lysate was purified on a Maxwell RSC 48 platform (Promega Corporation) according to the manufacturer's recommendations. Preparation of cDNA was performed using Quanta qScript cDNA SuperMix (VWR, Catalog #95048-500) according to the manufacturer's instructions. The relative levels of liver FGG mRNA were assessed by RT-qPCR in triplicate on a QuantStudio™ 6 Pro Real-Time PCR System using TaqMan assays for mouse FGG (ThermoFisher, assay #Mm00513575_m1) and the mouse housekeeping gene PPIA (ThermoFisher, assay #Mm02342430_g1) and PerfeCea® qPCR FastMix®, Low ROX™ (VWR, Catalog #101419-222). Data were normalized to the mean EGG mRNA level in animals receiving PBS. Results are shown in Table 67

On Day 0 (prior to dosing) and Day 21 blood was collected into tubes with 0.2 mL sodium citrate for collection of plasma. Plasma samples were analyzed for fibrinogen levels by ELISA (Molecular Innovations Mouse Fibrinogen Antigen ELISA kit, Cat #MFBGNKT). Results are shown in Table 68.

On Day 2, 9, and Day 21 blood was collected into tubes with no anti-coagulant abd serum collected. Clinical chemistry parameters containing ALT, AST, ALP, TBLL, GLU, BUN, and CREAT were analyzed at IDEXX Laboratories (IDEXX Laboratories, Test #62849). Results are shown in Table 69-74.

TABLE 66A
Example siRNA Sequences

| | | Sense Strand | | Antisense |
|---|---|---|---|---|
| | SEQ | Sequence | SEQ | Strand |
| | ID | (5′-3′) with | ID | Sequence |
| ETD# | NO | GalNAc moiety | NO | (5′-3′) |
| ETD01818 | 3641 | [ETL1]gsasugAf | 3677 | usAfscUfaCfcGfa |
| | | AfaGfAfuucggua | | AfuCfuUfuCfaUfc |
| | | guasusu | | susu |
| ETD01839 | 3642 | [ETL1]gsasagAf | 3678 | usAfscuaCfcGfaA |
| | | uucAfuuugauaag | | fuCfuUfuCfaUfcs |
| | | asusu | | usu |
| ETD01841 | 3643 | [ETL1]sgaugAfA | 3679 | usAfscUfacCfgaA |
| | | faGfAfuucgGfua | | fuCfuUfuCfaUfcs |
| | | Gfuasusu | | usu |
| ETD01852 | 3644 | [ETL1]sgaugAfA | 3680 | usGfsaUfgUfgAfa |
| | | faGfdAuUfCfggU | | AfaAfcUfuGfuCfa |
| | | faguasusu | | susu |
| ETD01926 | 3645 | [ETL1]sgaAfGfa | 3681 | usGfsaUfguGfaaa |
| | | uucAfuuugAfuAf | | aAfcUfuGfuCfasu |
| | | Afgasusu | | su |

TABLE 66B
Example siRNA BASE Sequences

| | | Sense Strand | | Antisense |
|---|---|---|---|---|
| | | Base | | Strand Base |
| | | Sequence | | Sequence |
| | SEQ | (5′ to 3′), | SEQ | (5′ to 3′), |
| siRNA | ID | without 3′ | ID | without 3′ |
| Name | NO: | overhangs | NO: | overhangs |
| ETD0181 | 3641 | GAUGAAAGAUUCG | 3677 | UACUACCGAAUCU |
| 8 | | GUAGUAUU | | UUCAUCUU |
| ETD0183 | 3642 | GAUGAAAGAUUCG | 3678 | UACUACCGAAUCU |
| 9 | | GUAGUAUU | | UUCAUCUU |
| ETD0184 | 3643 | GAUGAAAGAUUCG | 3679 | UACUACCGAAUCU |
| 1 | | GUAGUAUU | | UUCAUCUU |
| ETD0185 | 3644 | UGACAAGUUUUUC | 3680 | UGAUGUGAAAAAC |
| 2 | | ACAUCAUU | | UUGUCAUU |
| ETD0192 | 3645 | UGACAAGUUUUUC | 3681 | UGAUGUGAAAAAC |
| 6 | | ACAUCAUU | | UUGUCAUU |

TABLE 67
Relative FGG mRNA Levels in Livers of Mice

| | | | | Mean FGG mRNA |
|---|---|---|---|---|
| | | | Dose | (Normalized to |
| Group | n | Treatment | (ug) | Group 1, Day 14) |
| 1 | 4 | PBS | 0 | 1.00 |
| 2 | 4 | ETD01818 | 200 ug × 3 | 0.10 |
| 3 | 4 | ETD01839 | 200 ug × 3 | 0.04 |
| 4 | 4 | ETD01841 | 200 ug × 3 | 0.05 |
| 5 | 4 | ETD01852 | 200 ug × 3 | 0.10 |
| 6 | 4 | ETD01926 | 200 ug × 3 | 0.05 |

TABLE 68
Fibrinogen Levels in Plasma of Mice
treated with siRNAs targeting FGG

| | | | | Mean Fibrinogen | |
|---|---|---|---|---|---|
| | | | Dose | (Normalized, Day 0) | |
| Group | n | Treatment | (ug) | Day 0 | Day 7 |
| 1 | 4 | PBS | 0 | 1.00 | 0.63 |
| 2 | 4 | ETD01818 | 200 ug × 3 | 1.00 | 0.03 |
| 3 | 4 | ETD01839 | 200 ug × 3 | 1.00 | 0.01 |
| 4 | 4 | ETD01841 | 200 ug × 3 | 1.00 | 0.01 |
| 5 | 4 | ETD01852 | 200 ug × 3 | 1.00 | 0.15 |
| 6 | 4 | ETD01926 | 200 ug × 3 | 1.00 | 0.14 |

TABLE 69
Clinical Chemistry ALT in Mice
treated with siRNAs targeting FGG

| | | | Dose | Mean ALT (U/L) | | |
|---|---|---|---|---|---|---|
| Group | n | Treatment | (ug) | Day 2 | Day 9 | Day 21 |
| 1 | 4 | PBS | 0 | 23.25 | 24 | 27 |
| 2 | 4 | ETD01818 | 200 ug × 3 | 93.25 | 23 | 22.5 |
| 3 | 4 | ETD01839 | 200 ug × 3 | 85 | 30.75 | 27 |
| 4 | 4 | ETD01841 | 200 ug × 3 | 24.75 | 24 | 26.75 |
| 5 | 4 | ETD01852 | 200 ug × 3 | 24.25 | 33 | 30 |
| 6 | 4 | ETD01926 | 200 ug × 3 | 25.5 | 26.75 | 26.75 |

TABLE 70
Clinical Chemistry AST in Mice
treated with siRNAs targeting FGG

| | | | Dose | Mean AST (U/L) | | |
|---|---|---|---|---|---|---|
| Group | n | Treatment | (ug) | Day 2 | Day 9 | Day 21 |
| 1 | 4 | PBS | 0 | 60.5 | 53 | 64 |
| 2 | 4 | ETD01818 | 200 ug × 3 | 126 | 54.25 | 49.5 |
| 3 | 4 | ETD01839 | 200 ug × 3 | 122.25 | 56.75 | 55 |
| 4 | 4 | ETD01841 | 200 ug × 3 | 55.25 | 53 | 64 |
| 5 | 4 | ETD01852 | 200 ug × 3 | 56.25 | 60.75 | 59.25 |
| 6 | 4 | ETD01926 | 200 ug × 3 | 59.25 | 62.25 | 62 |

TABLE 71
Clinical Chemistry ALP in Mice
treated with siRNAs targeting FGG

| | | | Dose | Mean ALP (U/L) | | |
|---|---|---|---|---|---|---|
| Group | n | Treatment | (ug) | Day 2 | Day 9 | Day 21 |
| 1 | 4 | PBS | 0 | 129.75 | 118.5 | 94.25 |
| 2 | 4 | ETD01818 | 200 ug × 3 | 123.75 | 107 | 84 |
| 3 | 4 | ETD01839 | 200 ug × 3 | 115.5 | 96.75 | 109.75 |
| 4 | 4 | ETD01841 | 200 ug × 3 | 95.25 | 81 | 82 |
| 5 | 4 | ETD01852 | 200 ug × 3 | 120.25 | 118 | 101.25 |
| 6 | 4 | ETD01926 | 200 ug × 3 | 140.75 | 126.25 | 110.25 |

TABLE 72
Clinical Chemistry TBILI in Mice
treated with siRNAs targeting FGG

| | | | Dose | Mean TBILI (mg/dL) | | |
|---|---|---|---|---|---|---|
| Group | n | Treatment | (ug) | Day 2 | Day 9 | Day 21 |
| 1 | 4 | PBS | 0 | 0.175 | 0.2 | 0.2 |
| 2 | 4 | ETD01818 | 200 ug × 3 | 0.125 | 0.15 | 0.1 |
| 3 | 4 | ETD01839 | 200 ug × 3 | 0.15 | 0.2 | 0.125 |
| 4 | 4 | ETD01841 | 200 ug × 3 | 0.15 | 0.2 | 0.125 |
| 5 | 4 | ETD01852 | 200 ug × 3 | 0.1 | 0.15 | 0.15 |
| 6 | 4 | ETD01926 | 200 ug × 3 | 0.125 | 0.2 | 0.175 |

TABLE 73
Clinical Chemistry BUN in Mice
treated with siRNAs targeting FGG

| | | | Dose | Mean BUN (mg/dL) | | |
|---|---|---|---|---|---|---|
| Group | n | Treatment | (ug) | Day 2 | Day 9 | Day 21 |
| 1 | 4 | PBS | 0 | 25 | 25.5 | 23.5 |
| 2 | 4 | ETD01818 | 200 ug × 3 | 24.25 | 25 | 21 |
| 3 | 4 | ETD01839 | 200 ug × 3 | 22.5 | 26.5 | 20.25 |
| 4 | 4 | ETD01841 | 200 ug × 3 | 25.75 | 24.5 | 24 |
| 5 | 4 | ETD01852 | 200 ug × 3 | 26 | 25.75 | 23.75 |
| 6 | 4 | ETD01926 | 200 ug × 3 | 24 | 22 | 23 |

TABLE 74
Clinical Chemistry BUN in Mice
treated with siRNAs targeting FGG

| | | | Dose | Mean CREAT (mg/dL) | | |
|---|---|---|---|---|---|---|
| Group | n | Treatment | (ug) | Day 2 | Day 9 | Day 21 |
| 1 | 4 | PBS | 0 | 0.075 | 0 | 0.075 |
| 2 | 4 | ETD01818 | 200 ug × 3 | 0.1 | 0.025 | 0.025 |
| 3 | 4 | ETD01839 | 200 ug × 3 | 0.1 | 0.05 | 0.075 |
| 4 | 4 | ETD01841 | 200 ug × 3 | 0.175 | 0.025 | 0.05 |
| 5 | 4 | ETD01852 | 200 ug × 3 | 0.1 | 0.025 | 0.075 |
| 6 | 4 | ETD01926 | 200 ug × 3 | 0.1 | 0 | 0.075 |

### Example 22: Determining the Activity of Species Cross-Reactive siRNAs Targeting FGG in Mice

3 groups (n=4/group) of 8-week-old male ICR mice (Invigo) were utilized in this study. On Study Day 0, Group 1 mice were injected subcutaneously with 100 μL of sterile PBS, Group 2 mice were subcutaneously injected with 60 μg of ETD01811 in 100 μL of sterile PBS, and Group 3 mice were subcutaneously injected with 200 ug ETD01818 in 100 μL of sterile PBS. On Study Day 14, the animals from all Groups were anesthetized and then euthanized. A liver sample was collected from all animals and placed in RNAlater™ Stabilization Solution (Thermo Fisher, Catalog #AM7020). The liver samples were processed in homogenization buffer (Maxwell RSC simplyRNA Tissue Kit) using Soft Tissue Homogenizing Kit CK14 (Bertin Instruments, catalog #P00093 3-LYSK0-A) in a Percellys 24 tissue homogenizer (Bertin Instruments) set at 5000 rpm for two 10 second cycles. Total RNA from the liver lysate was purified on a Maxwell RSC 48 platform (Promega Corporation) according to the manufacturer's recommendations. The relative level of FGG mRNA in each liver sample was assessed by RT-qPCR on a QuantStudio 6 Pro instrument (Applied Biosystems) using TaqMan assays for mouse FGG (ThermoFisher, assay #Mm00513575 ml) and the mouse housekeeping gene PPIA (ThermoFisher, assay #Mm02342430 g1), and then normalized to the mean value of the control mice (Group 1) using the delta-delta Ct method.

The results of the liver mRNA analyses are shown in Table 75 below. Animals treated with ETL1-targeted siRNA (ETD01811, Group 2) had 78% relative knockdown while ETL17-targeted siRNA (ETD01818, Group 3) had 83% knockdown of liver FGG mRNA levels compared with mice injected with PBS (Group 1).

TABLE 75
Day 14 FGG mRNA liver levels in mice
treated with siRNAs targeting FGG

| Group # | Treatment | Mean |
|---|---|---|
| 1 | PBS | 1.00 |
| 2 | ETD01811 | 0.22 |
| 3 | ETD01818 | 0.17 |

### Example 23: Oligonucleotide Synthesis

Oligonucleotides such as siRNAs may be synthesized according to phosphoramidite technology on a solid phase. For example, a K&A oligonucleotide synthesizer may be used. Syntheses may be performed on a solid support made of controlled pore glass (CPG, 500 Å or 600 Å, obtained from AM Chemicals, Oceanside, CA, USA). All 2′-Ome and 2′-F phosphoramidites may be purchased from Hongene Biotech (Union City, CA, USA). All phosphoramidites may be dissolved in anhydrous acetonitrile (100 mM) and molecular sieves (3 Å) may be added. 5-Benzylthio-1H-tetrazole (BTT, 250 mM in acetonitrile) or 5-Ethylthio-1H-tetrazole (ETT, 250 mM in acetonitrile) may be used as activator solution. Coupling times may be 9-18 min (e.g. with a GalNAc such as ETL17), 6 min (e.g. with 2′Ome and 2′F). In order to introduce phosphorothioate linkages, a 100 mM solution of 3-phenyl 1,2,4-dithiazoline-5-one (POS, obtained from PolyOrg, Inc., Leominster, Mass., USA) in anhydrous acetonitrile may be employed.

After solid phase synthesis, the dried solid support may be treated with a 1:1 volume solution of 40 wt. % methylamine in water and 28% ammonium hydroxide solution (Aldrich) for two hours at 30° C. The solution may be evaporated and the solid residue may be reconstituted in water and purified by anionic exchange HPLC using a TKSgel SuperQ-5PW 13u column. Buffer A may be 20 mM Tris, 5 mM EDTA, pH 9.0 and contained 20% Acetonitrile and buffer B may be the same as buffer A with the addition of 1 M sodium chloride. UV traces at 260 nm may be recorded. Appropriate fractions may be pooled then desalted using Sephadex G-25 medium.

Equimolar amounts of sense and antisense strand may be combined to prepare a duplex. The duplex solution may be prepared in 0.1×PBS (Phosphate-Buffered Saline, 1×, Gibco). The duplex solution may be annealed at 95° C. for 5 min, and cooled to room temperature slowly. Duplex concentration may be determined by measuring the solution absorbance on a UV-Vis spectrometer at 260 nm in 0.1×PBS. For some experiments, a conversion factor may be calculated from an experimentally determined extinction coefficient.

### Example 24: GalNAc Ligand for Hepatocyte Targeting of Oligonucleotides

Without limiting the disclosure to these individual methods, there are at least two general methods for attachment of multivalent N-acetylgalactosamine (GalNAc) ligands to oligonucleotides: solid or solution-phase conjugations. GalNAc ligands may be attached to solid phase resin for 3′ conjugation or at the 5′ terminus using GalNAc phosphoramidite reagents. GalNAc phosphoramidites may be coupled on solid phase as for other nucleosides in the oligonucleotide sequence at any position in the sequence. Reagents for GalNAc conjugation to oligonucleotides are shown in Table 76.

TABLE 76
GalNAc Conjugation Reagents

| Type of | |
|---|---|
| conjugation | Structure |
| Solid phase 3′ attachment where squiggly line is rest of oligonucleotide chain and right-most OH is where attachment′ to solid phase is. | 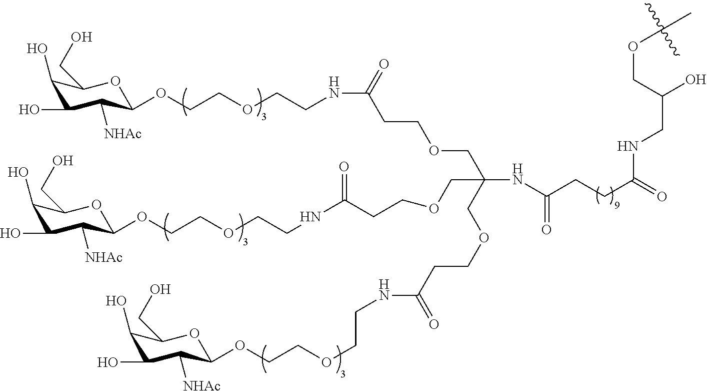 |
| | This GalNAc ligand may be referred to as “GalNAc23” or “GalNAc#23.” |
| Solid phase 5′ attachment phosphoramidite | 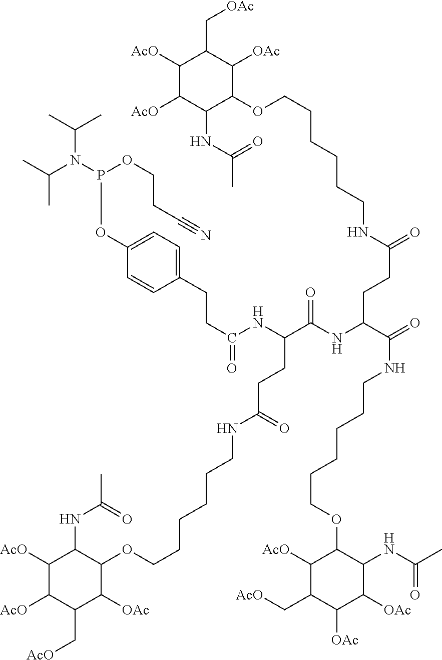 [SMILES: CC(=O)NC1C(OCCCCCCNC(=O)CCC(NC(=O)CCc2ccc(OP(OCCC#N)N(C(C)C)C(C)C)cc2)C(=O)NC(CCC(=O)NCCCCCCOC2C(NC(C)=O)C(OC(C)=O)C(OC(C)=O)C(COC(C)=O)C2OC(C)=O)C(=O)NCCCCCCOC2C(NC(C)=O)C(OC(C)=O)C(OC(C)=O)C(COC(C)=O)C2OC(C)=O)C(OC(C)=O)C(COC(C)=O)C(OC(C)=O)C1OC(C)=O] |
| Solid phase 5′ attachment Phosphoramidite | 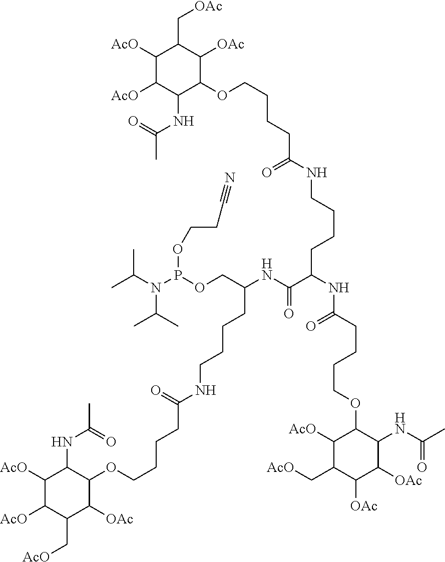 [SMILES: CC(=O)NC1C(OCCCCC(=O)NCCCCC(COP(OCCC#N)N(C(C)C)C(C)C)NC(=O)C(CCCCNC(=O)CCCCOC2C(NC(C)=O)C(OC(C)=O)C(OC(C)=O)C(COC(C)=O)C2OC(C)=O)NC(=O)CCCCOC2C(NC(C)=O)C(OC(C)=O)C(OC(C)=O)C(COC(C)=O)C2OC(C)=O)C(OC(C)=O)C(COC(C)=O)C(OC(C)=O)C1OC(C)=O] |
| Solution phase Carboxylic acid for amide coupling anywhere on oligonucleotide | 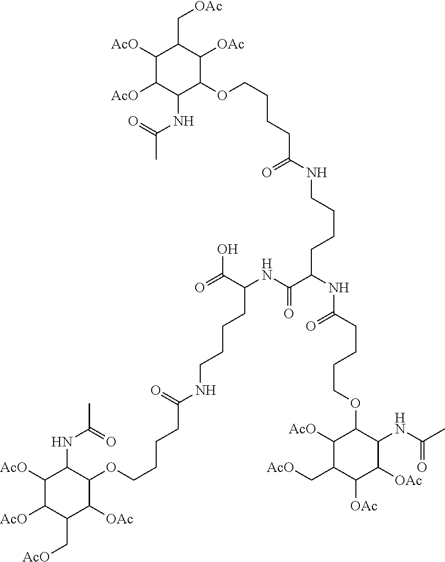 [SMILES: CC(=O)NC1C(OCCCCC(=O)NCCCCC(NC(=O)C(CCCCNC(=O)CCCCOC2C(NC(C)=O)C(OC(C)=O)C(OC(C)=O)C(COC(C)=O)C2OC(C)=O)NC(=O)CCCCOC2C(NC(C)=O)C(OC(C)=O)C(OC(C)=O)C(COC(C)=O)C2OC(C)=O)C(=O)O)C(OC(C)=O)C(COC(C)=O)C(OC(C)=O)C1OC(C)=O] |
| | Where Ac is an acetyl group or other hydroxyl protecting group that can be removed under basic, acid or reducing conditions. |

In solution phase conjugation, the oligonucleotide sequence—including a reactive conjugation site—is formed on the resin. The oligonucleotide is then removed from the resin and GalNAc is conjugated to the reactive site.

The carboxy GalNAc derivatives may be coupled to amino-modified oligonucleotides. The peptide coupling conditions are known to the skilled in the art using a carbodiimide coupling agent like DCC (N,N′-Dicyclohexylcarbodiimide), EDC (N-(3-dimethylaminopropyl)-N′-ethylcarbodiimide) or EDC·HCl (N-(3-dimethylaminopropyl)-N′-ethylcarbodiimide hydrochloride and an additive like HOBt (1-hydroxybenztriazole), HOSu (N-hydroxysuccinimide), TBTU (N,N,N′,N′-Tetramethyl-O-(benzotriazol-1-yl)uronium tetrafluoroborate, HBTU (2-(1H-benzotriazol-1-yl)-1,1,3,3-tetramethyluronium hexafluorophosphate) or HOAt (1-Hydroxy-7-azabenzotriazole and common combinations thereof such as TBTU/HOBt or HBTU/HOAt to form activated amine-reactive esters.

Amine groups may be incorporated into oligonucleotides using a number of known, commercially available reagents at the 5′ terminus, 3′ terminus or anywhere in between.

Non-limiting examples of reagents for oligonucleotide synthesis to incorporate an amino group include:
5′ attachment:
6-(4-Monomethoxytritylamino)hexyl-(2-cyanoethyl)-(N,N-diisopropyl)-phosphoramidite CAS Number: 114616-27-2
5′-Amino-Modifier TEG CE-Phosphoramidite
10-(O-trifluoroacetamido-N-ethyl)-triethyleneglycol-1-[(2-cyanoethyl)-(N,N-diisopropyl)]-phosphoramidite
3′ attachment:
3′-Amino-Modifier Serinol CPG
3-Dimethoxytrityloxy-2-(3-(fluorenylmethoxycarbonylamino)propanamido)propyl-1-O-succinyl-long chain alkylamino-CPG (where CPG stands for controlled-pore glass and is the solid support)
Amino-Modifier Serinol Phosphoramidite
3-Dimethoxytrityloxy-2-(3-(fluorenylmethoxycarbonylamino)propanamido)propyl-1-O-(2-cyanoethyl)-(N,N-diisopropyl)-phosphoramidite

Internal (base modified):
Amino-Modifier C6 dT
5′-Dimethoxytrityl-5-[N-(trifluoroacetylaminohexyl)-3-acrylimido]-2′-deoxyUridine,3′-[(2-cyanoethyl)-(N,N-diisopropyl)]-phosphoramidite. CAS Number: 178925-21-8

Solution phase conjugations may occur after oligonucleotide synthesis via reactions between non-nucleosidic nucleophilic functional groups that are attached to the oligonucleotide and electrophilic GalNAc reagents. Examples of nucleophilic groups include amines and thiols, and examples of electrophilic reagents include activated esters (e.g. N-hydroxysuccinimide, pentafluorophenyl) and maleimides.

### Example 25: GalNAc Ligands for Hepatocyte Targeting of Oligonucleotides

Without limiting the disclosure to these individual methods, there are at least two general methods for attachment of multivalent N-acetylgalactosamine (GalNAc) ligands to oligonucleotides: solid or solution-phase conjugations. GalNAc ligands may be attached to solid phase resin for 3′ conjugation or at the 5′ terminus using GalNAc phosphoramidite reagents. GalNAc phosphoramidites may be coupled on solid phase as for other nucleosides in the oligonucleotide sequence at any position in the sequence. A non-limiting example of a phosphoramidite reagent for GalNAc conjugation to a 5′ end oligonucleotide is shown in Table 77.

TABLE 77
GalNAc Conjugation Reagent

| Type of | |
|---|---|
| conjugation | Structure |
| Solid phase 5′ attachment phosphoramidite | 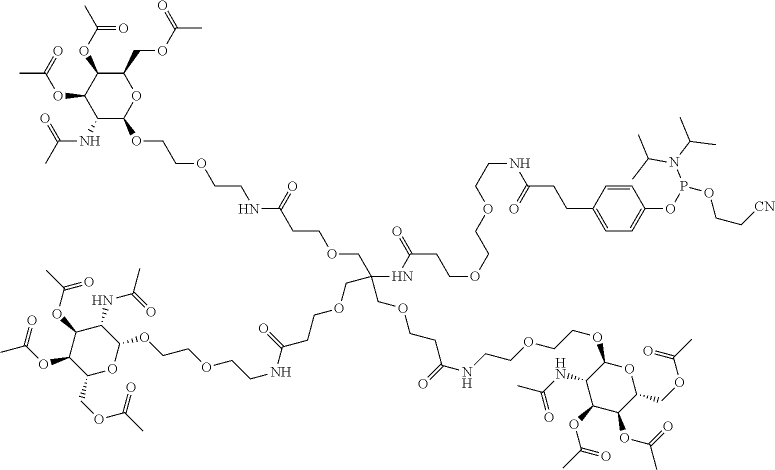 [SMILES: CC(=O)N[C@@H]1[C@H](OCCOCCNC(=O)CCOCC(COCCC(=O)NCCOCCO[C@H]2O[C@H](COC(C)=O)[C@@H](OC(C)=O)[C@@H](OC(C)=O)[C@@H]2NC(C)=O)(COCCC(=O)NCCOCCO[C@@H]2O[C@H](COC(C)=O)[C@H](OC(C)=O)[C@H](OC(C)=O)[C@H]2NC(C)=O)NC(=O)CCOCCOCCNC(=O)CCc2ccc(OP(OCCC#N)N(C(C)C)C(C)C)cc2)O[C@H](COC(C)=O)[C@@H](OC(C)=O)[C@H]1OC(C)=O] |

The following includes examples of synthesis reactions used to create a GalNAc moiety:

Scheme for the preparation of Nacegal-Linker-TMSOTf

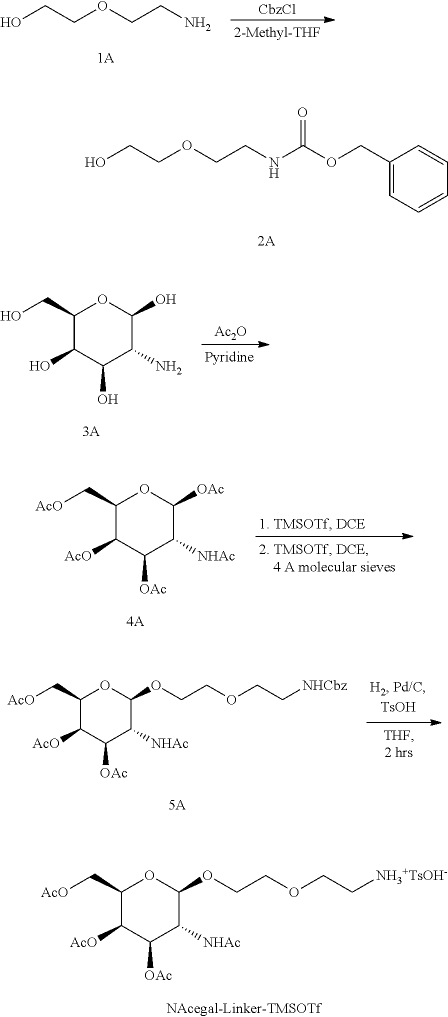

### General Procedure for Preparation of Compound 2A

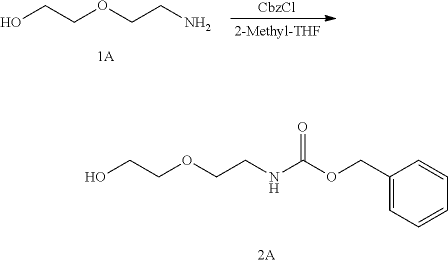

To a solution of Compound 1A (500 g, 4.76 mol, 476 mL) in 2-Methyl-THF (2.00 L) is added CbzCl (406 g, 2.38 mol, 338 mL) in 2-Methyl-THF (750 mL) dropwise at 0° C. The mixture is stirred at 25° C. for 2 hrs under N₂ atmosphere. TLC (DCM:MeOH=20:1, PMA) may indicate CbzCl is consumed completely and one new spot (R_{f}=0.43) formed. The reaction mixture is added HCl/EtOAc (1 N, 180 mL) and stirred for 30 mins, white solid is removed by filtration through celite, the filtrate is concentrated under vacuum to give Compound 2A (540 g, 2.26 mol, 47.5% yield) as a pale yellow oil and used into the next step without further purification. ¹H NMR: δ 7.28-7.41 (m, 5H), 5.55 (br s, 1H), 5.01-5.22 (m, 2H), 3.63-3.80 (m, 2H), 3.46-3.59 (m, 4H), 3.29-3.44 (m, 2H), 2.83-3.02 (m, 1H).

### General Procedure for Preparation of Compound 4A

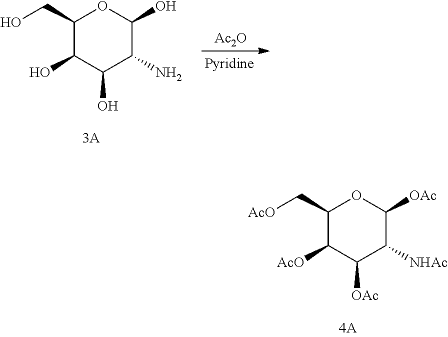 [SMILES: "Ac2O" ; N[C@@H]1[C@@H](O)[C@@H](O)[C@@H](CO)O[C@H]1O ; CC(=O)N[C@H]1[C@H](OC(C)=O)O[C@H](COC(C)=O)[C@H](OC(C)=O)[C@@H]1OC(C)=O]

To a solution of Compound 3A (1.00 kg, 4.64 mol, HCl) in pyridine (5.00 L) is added acetyl acetate (4.73 kg, 46.4 mol, 4.34 L) dropwise at 0° C. under N₂ atmosphere. The mixture is stirred at 25° C. for 16 hrs under N₂ atmosphere. TLC (DCM:MeOH=20:1, PMA) indicated Compound 3A is consumed completely and two new spots (R_{f}=0.35) formed. The reaction mixture is added to cold water (30.0 L) and stirred at 0° C. for 0.5 hr, white solid formed, filtered and dried to give Compound 4A (1.55 kg, 3.98 mol, 85.8% yield) as a white solid and used in the next step without further purification. ¹H NMR: δ 7.90 (d, J=9.29 Hz, 1H), 5.64 (d, J=8.78 Hz, 1H), 5.26 (d, J=3.01 Hz, 1H), 5.06 (dd, J=11.29, 3.26 Hz, 1H), 4.22 (t, J=6.15 Hz, 1H), 3.95-4.16 (m, 3H), 2.12 (s, 3H), 2.03 (s, 3H), 1.99 (s, 3H), 1.90 (s, 3H), 1.78 (s, 3H).

### General Procedure for Preparation of Compound 5A

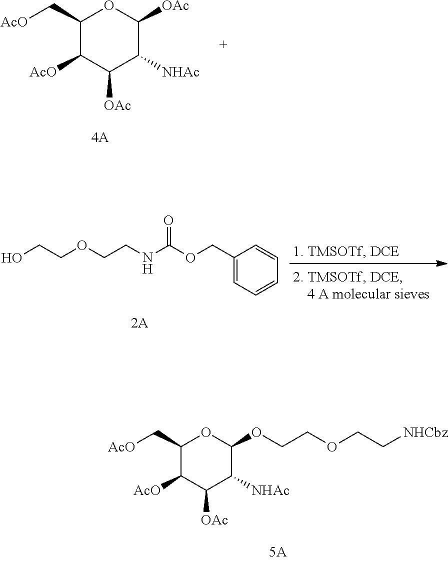

To a solution of Compound 4A (300 g, 771 mmol) in DCE (1.50 L) is added TMSOTf (257 g, 1.16 mol, 209 mL) and stirred for 2 hrs at 60° C., and then stirred for 1 hr at 25° C. Compound 2A (203 g, 848 mmol) is dissolved in DCE (1.50 L) and added 4 powder molecular sieves (150 g) stirring for 30 mins under N₂ atmosphere. Then the solution of Compound 4A in DCE is added dropwise to the mixture at 0° C. The mixture is stirred at 25° C. for 16 hrs under N₂ atmosphere. TLC (DCM:MeOH=25:1, PMA) indicated Compound 4A is consumed completely and new spot (R_{f}=0.24) formed. The reaction mixture is filtered and washed with sat. NaHCO₃ (2.00 L), water (2.00 L) and sat. brine (2.00 L). The organic layer is dried over anhydrous Na₂SO₄, filtered and concentrated under reduced pressure to give a residue. The residue is triturated with 2-Me-THE/heptane (5/3, v/v, 1.80 L) for 2 hrs, filtered and dried to give Compound 5A (225 g, 389 mmol, 50.3% yield, 98.4% purity) as a white solid. ¹H NMR: δ 7.81 (d, J=9.29 Hz, 1H), 7.20-7.42 (m, 6H), 5.21 (d, J=3.26 Hz, 1H), 4.92-5.05 (m, 3H), 4.55 (d, J=8.28 Hz, 1H), 3.98-4.07 (m, 3H), 3.82-3.93 (m, 1H), 3.71-3.81 (m, 1H), 3.55-3.62 (m, 1H), 3.43-3.53 (m, 2H), 3.37-3.43 (m, 2H), 3.14 (q, J=5.77 Hz, 2H), 2.10 (s, 3H), 1.99 (s, 3H), 1.89 (s, 3H), 1.77 (s, 3H).

### General Procedure for Preparation of Nacegal-Linker-Tosylate Salt

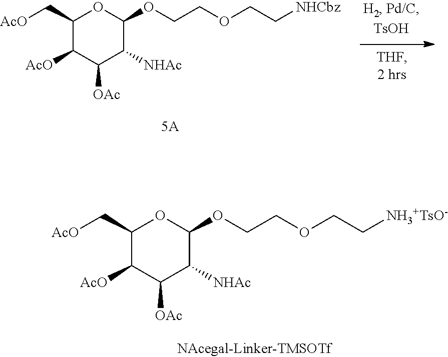

To a solution of Compound 5A (200 g, 352 mmol) in THF (1.0 L) is added dry Pd/C (15.0 g, 10% purity) and TsOH (60.6 g, 352 mmol) under N₂ atmosphere. The suspension is degassed under vacuum and purged with H₂ several times. The mixture is stirred at 25° C. for 3 hrs under H₂ (45 psi) atmosphere. TLC (DCM:MeOH=10:1, PMA) indicated Compound 5A is consumed completely and one new spot (R_{f}=0.04) is formed. The reaction mixture is filtered and concentrated (≤40° C.) under reduced pressure to give a residue. Diluted with anhydrous DCM (500 mL, dried overnight with 4 Å molecular sieves (dried at 300° C. for 12 hrs)) and concentrate to give a residue and run Karl Fisher (KF) to check for water content. This is repeated 3 times with anhydrous DCM (500 mL) dilutions and concentration to give Nacegal-Linker-TMSOTf (205 g, 95.8% yield, TsOH salt) as a foamy white solid. ¹H NMR: δ 7.91 (d, J=9.03 Hz, 1H), 7.53-7.86 (m, 2H), 7.49 (d, J=8.03 Hz, 2H), 7.13 (d, J=8.03 Hz, 2H), 5.22 (d, J=3.26 Hz, 1H), 4.98 (dd, J=11.29, 3.26 Hz, 1H), 4.57 (d, J=8.53 Hz, 1H), 3.99-4.05 (m, 3H), 3.87-3.94 (m, 1H), 3.79-3.85 (m, 1H), 3.51-3.62 (m, 5H), 2.96 (br t, J=5.14 Hz, 2H), 2.29 (s, 3H), 2.10 (s, 3H), 2.00 (s, 3H), 1.89 (s, 3H), 1.78 (s, 3H).

### Scheme for the Preparation of TRIS-PEG2-CBZ

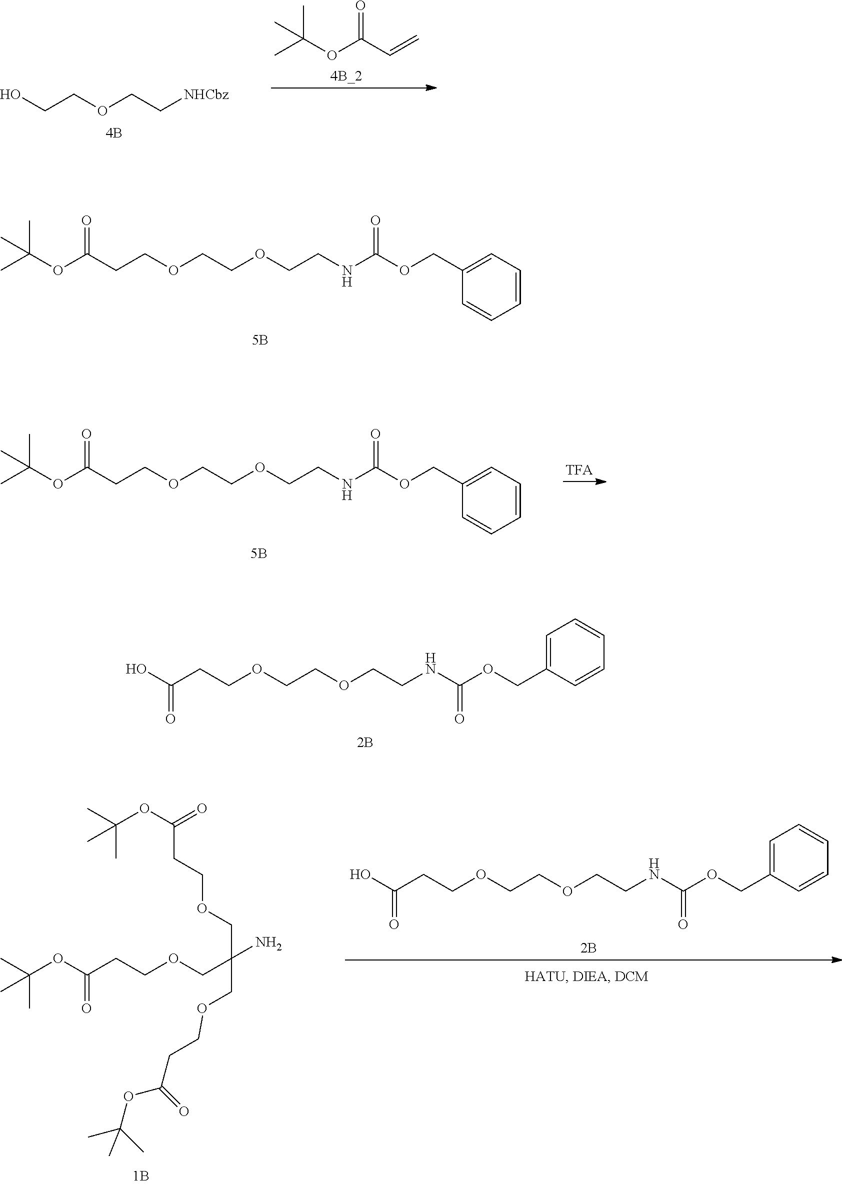

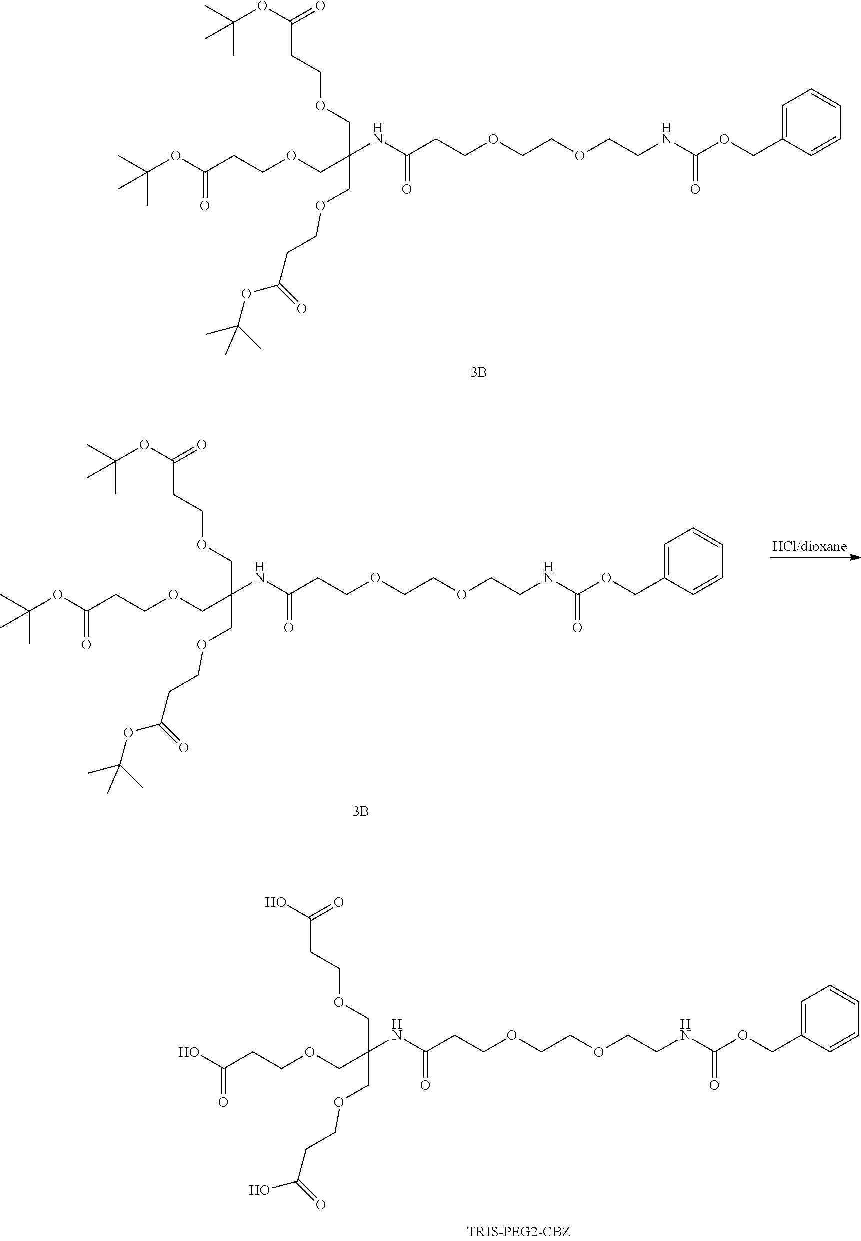 [SMILES: CC(C)(C)OC(=O)CCOCC(COCCC(=O)OC(C)(C)C)(COCCC(=O)OC(C)(C)C)NC(=O)CCOCCOCCNC(=O)OCc1ccccc1 ; CC(C)(C)OC(=O)CCOCC(COCCC(=O)OC(C)(C)C)(COCCC(=O)OC(C)(C)C)NC(=O)CCOCCOCCNC(=O)OCc1ccccc1 ; O=C(O)CCOCC(COCCC(=O)O)(COCCC(=O)O)NC(=O)CCOCCOCCNC(=O)OCc1ccccc1]

### General Procedure for Preparation of Compound 5B

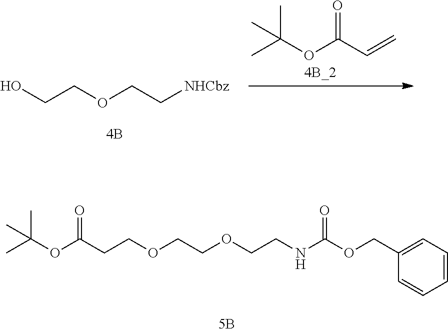

To a solution of Compound 4B (400 g, 1.67 mol, 1.00 eq) and NaOH (10 M, 16.7 mL, 0.10 eq) in THF (2.00 L) is added Compound 4B_2 (1.07 kg, 8.36 mol, 1.20 L, 5.00 eq), the mixture is stirred at 30° C. for 2 hrs. LCMS showed the desired MS is given. Five batches of solution are combined to one batch, then the mixture is diluted with water (6.00 L), extracted with ethyl acetate (3.00 L*3), the combined organic layer is washed with brine (3.00 L), dried over Na₂SO₄, filtered and concentrated under vacuum. The crude is purified by column chromatography (SiO₂, petroleum ether:ethyl acetate=100:1-10:1, R_{f}=0.5) to give Compound 5B (2.36 kg, 6.43 mol, 76.9% yield) as light yellow oil. HNMR: δ 7.31-7.36 (m, 5H), 5.38 (s, 1H), 5.11-5.16 (m, 2H), 3.75 (t, J=6.4 Hz), 3.54-3.62 (m, 6H), 3.39 (d, J=5.2 Hz), 2.61 (t, J=6.0 Hz).

### General procedure for preparation of 3-oxo-1-phenyl-2,7,10-trioxa-4-azatridecan-13-oic acid (Compound 2B below)

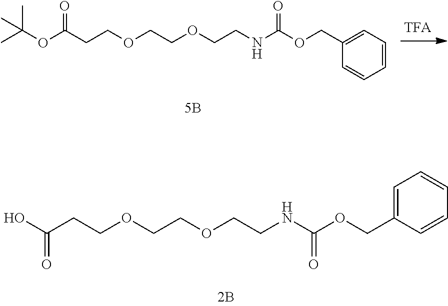 [SMILES: CC(C)(C)OC(=O)CCOCCOCCNC(=O)OCc1ccccc1 ; O=C(O)CCOCCOCCNC(=O)OCc1ccccc1]

To a solution of Compound 5B (741 g, 2.02 mol, 1.00 eq) in DCM (2.80 L) is added TFA (1.43 kg, 12.5 mol, 928 mL, 6.22 eq), the mixture is stirred at 25° C. for 3 hrs. LCMS showed the desired MS is given. The mixture is diluted with DCM (5.00 L), washed with water (3.00 L*3), brine (2.00 L), the combined organic layer is dried over Na₂SO₄, filtered and concentrated under vacuum to give Compound 2B (1800 g, crude) as light yellow oil. HNMR: δ 9.46 (s, 5H), 7.27-7.34 (m, 5H), 6.50-6.65 (m, 1H), 5.71 (s, 1H), 5.10-5.15 (m, 2H), 3.68-3.70 (m, 14H), 3.58-3.61 (m, 6H), 3.39 (s, 2H), 2.55 (s, 6H), 2.44 (s, 2H).

### General Procedure for Preparation of Compound 3B

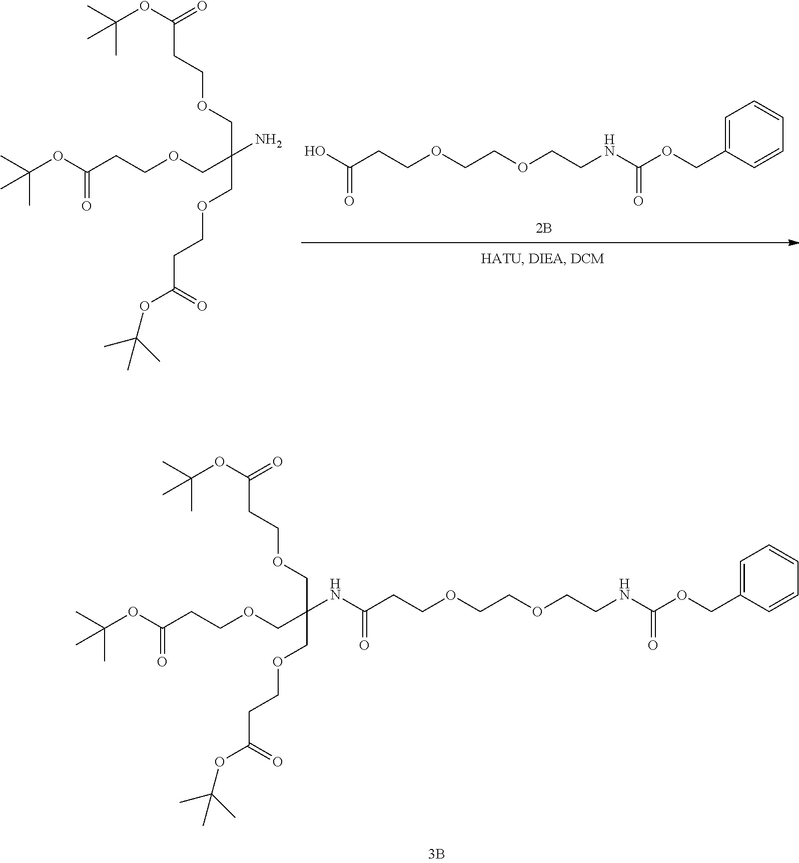 [SMILES: CC(C)(C)OC(=O)CCOCC(N)(COCCC(=O)OC(C)(C)C)COCCC(=O)OC(C)(C)C ; O=C(O)CCOCCOCCNC(=O)OCc1ccccc1 ; CC(C)(C)OC(=O)CCOCC(COCCC(=O)OC(C)(C)C)(COCCC(=O)OC(C)(C)C)NC(=O)CCOCCOCCNC(=O)OCc1ccccc1]

To a solution of Compound 2B (375 g, 999 mmol, 83.0% purity, 1.00 eq) in DCM (1.80 L) is added HATU (570 g, 1.50 mol, 1.50 eq) and DIEA (258 g, 2.00 mol, 348 mL, 2.00 eq) at 0° C., the mixture is stirred at 0° C. for 30 min, then Compound 1B (606 g, 1.20 mol, 1.20 eq) is added, the mixture is stirred at 25° C. for 1 hr. LCMS showed desired MS is given. The mixture is combined to one batch, then the mixture is diluted with DCM (5.00 L), washed with 1 N HCl aqueous solution (2.00 L*2), then the organic layer is washed with saturated Na₂CO₃ aqueous solution (2.00 L*2) and brine (2.00 L), the organic layer is dried over Na₂SO₄, filtered and concentrated under vacuum to give Compound 3B (3.88 kg, crude) as yellow oil.

### General Procedure for Preparation of TRIS-PEG2-CBZ.

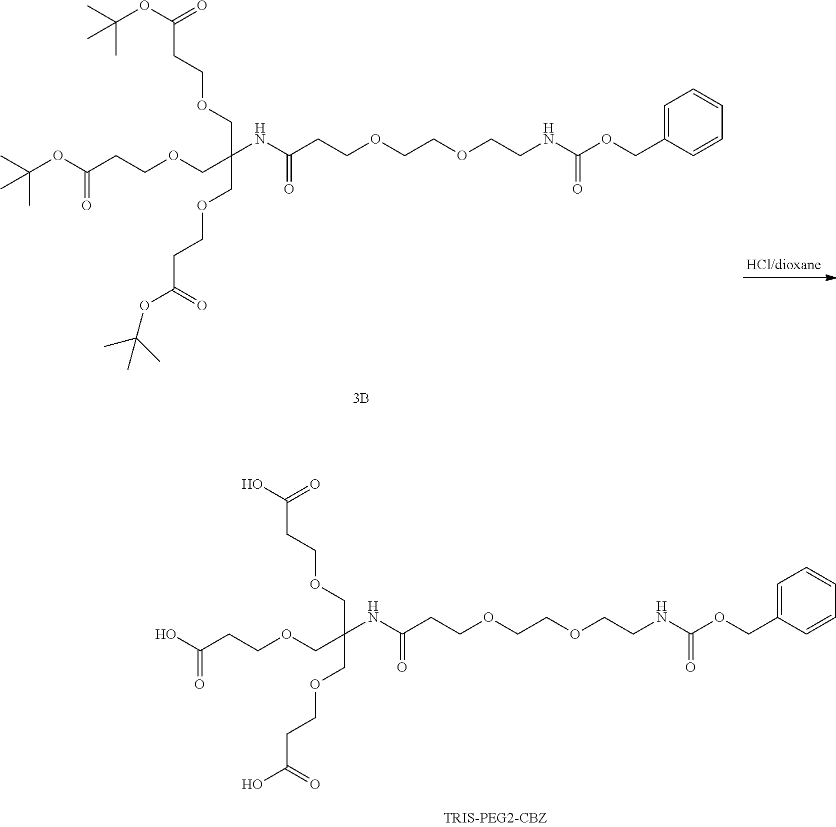 [SMILES: CC(C)(C)OC(=O)CCOCC(COCCC(=O)OC(C)(C)C)(COCCC(=O)OC(C)(C)C)NC(=O)CCOCCOCCNC(=O)OCc1ccccc1 ; O=C(O)CCOCC(COCCC(=O)O)(COCCC(=O)O)NC(=O)CCOCCOCCNC(=O)OCc1ccccc1]

A solution of Compound 3B (775 g, 487 mmol, 50.3% purity, 1.00 eq) in HCl/dioxane (4 M, 2.91 L, 23.8 eq) is stirred at 25° C. for 2 hrs. LCMS showed the desired MS is given. The mixture is concentrated under vacuum to give a residue. Then the combined residue is diluted with DCM (5.00 L), adjusted to pH=8 with 2.5 M NaOH aqueous solution, and separated. The aqueous phase is extracted with DCM (3.00 L) again, then the aqueous solution is adjusted to pH=3 with 1 N HCl aqueous solution, then extracted with DCM (5.00 L*2), the combined organic layer is washed with brine (3.00 L), dried over Na₂SO₄, filtered and concentrated under vacuum. The crude is purified by column chromatography (SiO₂, DCM:MeOH=0:1-12:1, 0.1% HOAc, R_{f}=0.4). The residue is diluted with DCM (5.00 L), adjusted to pH=8 with 2.5 M NaOH aqueous solution, separated, the aqueous solution is extracted with DCM (3.00 L) again, then the aqueous solution is adjusted to pH=3 with 6 N HCl aqueous solution, extracted with DCM:MeOH=10:1 (5.00 L*2), the combined organic layer is washed with brine (2.00 L), dried over Na₂SO₄, filtered and concentrated under vacuum to give a residue. Then the residue is diluted with MeCN (5.00 L), concentrated under vacuum, repeat this procedure twice to remove water to give TRIS-PEG2-CBZ (1.25 kg, 1.91 mol, 78.1% yield, 95.8% purity) as light yellow oil. ¹HNMR: 400 MHz, MeOD, δ 7.30-7.35 (5H), 5.07 (s, 2H), 3.65-3.70 (m, 16H), 3.59 (s, 4H), 3.45 (t, J=5.6 Hz), 2.51 (t, J=6.0 Hz), 2.43 (t, 6.4 Hz).

Scheme for the preparation of TriNGal-TRIS-Peg2-Phosph 8c

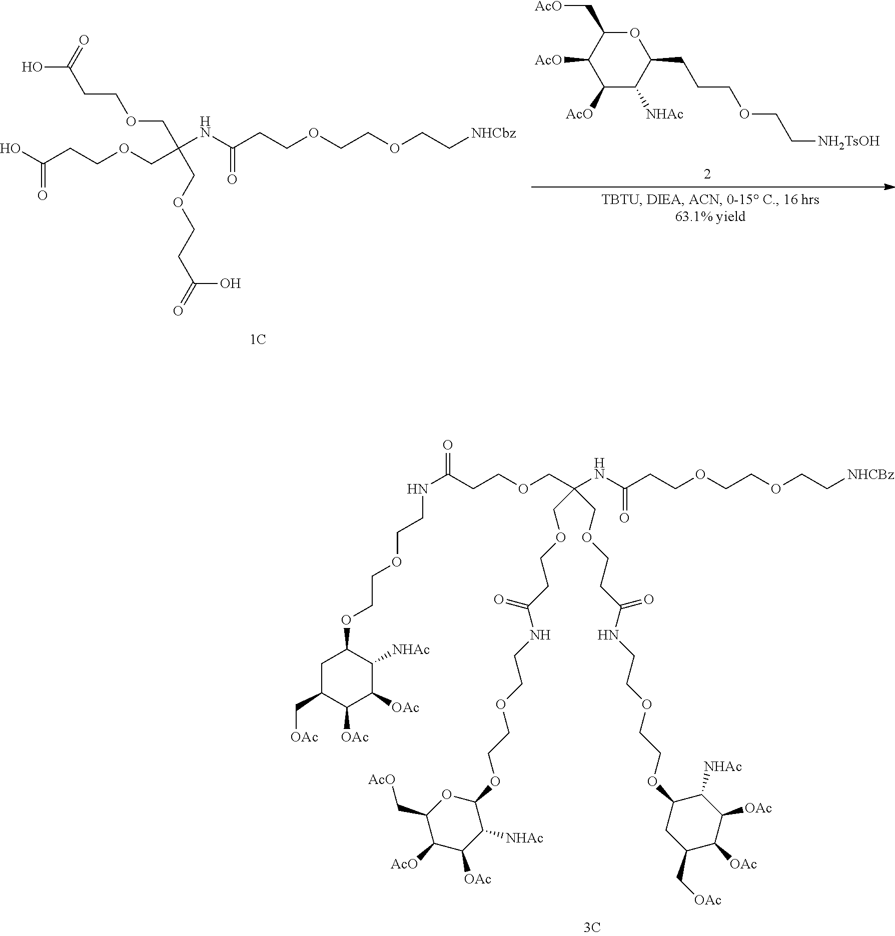

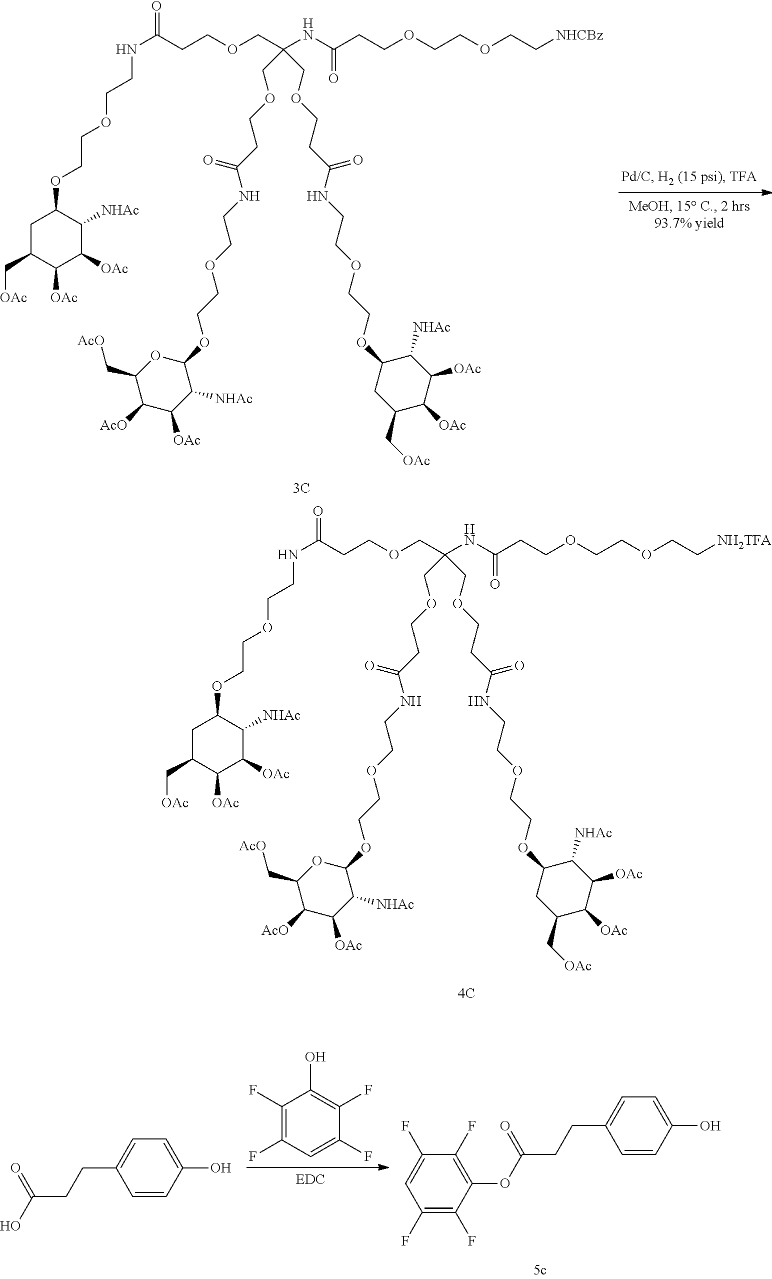

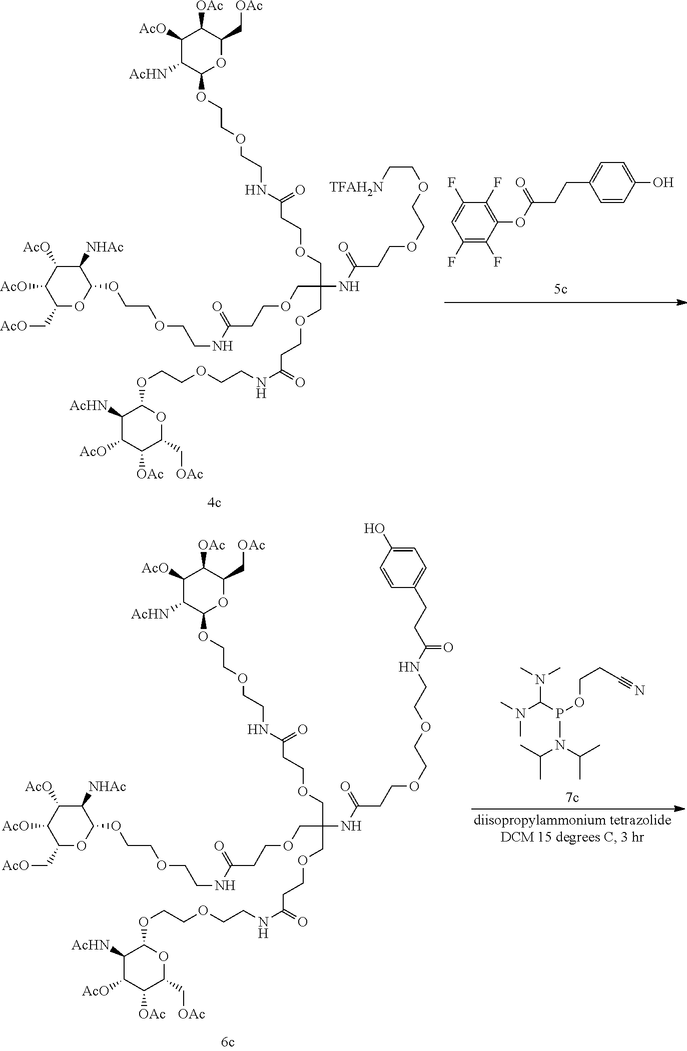

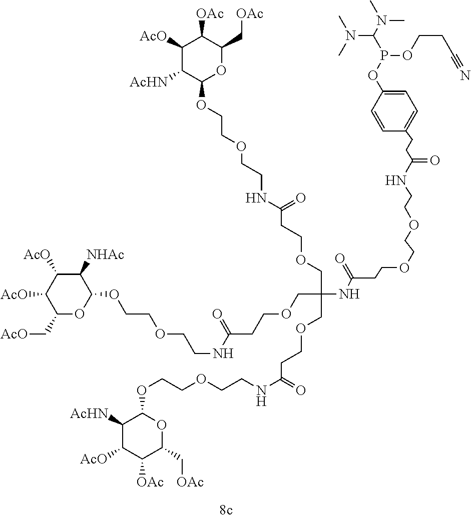 [SMILES: CC(=O)N[C@H]1[C@H](OCCOCCNC(=O)CCOCC(COCCC(=O)NCCOCCO[C@@H]2O[C@H](COC(C)=O)[C@H](OC(C)=O)[C@H](OC(C)=O)[C@H]2NC(C)=O)(COCCC(=O)NCCOCCO[C@@H]2O[C@H](COC(C)=O)[C@H](OC(C)=O)[C@H](OC(C)=O)[C@H]2NC(C)=O)NC(=O)CCOCCOCCNC(=O)CCc2ccc(OP(OCCC#N)C(N(C)C)N(C)C)cc2)O[C@H](COC(C)=O)[C@H](OC(C)=O)[C@@H]1OC(C)=O]

### TriGNal-TRIS-Peg2-Phosph 8c

### General Procedure for Preparation of Compound 3C

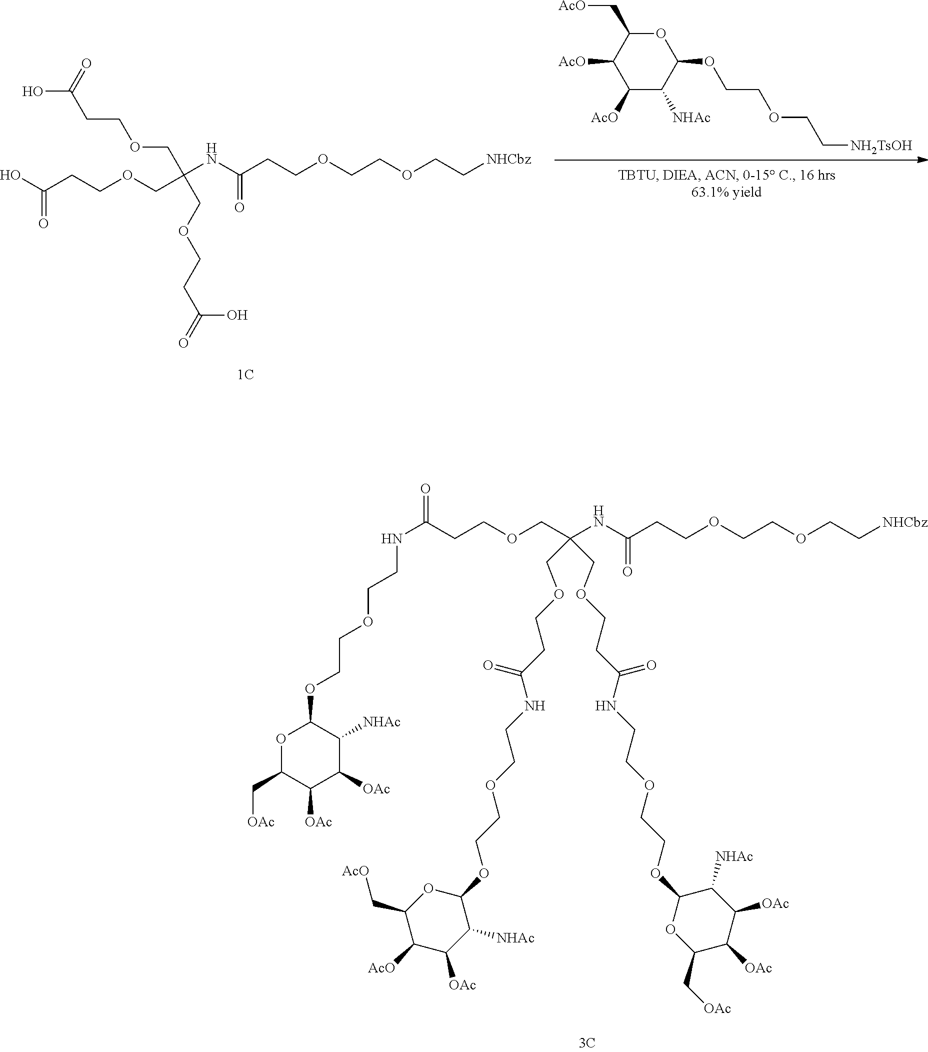

To a solution of Compound 1C (155 g, 245 mmol, 1.00 eq) in can (1500 mL) is added TBTU (260 g, 811 mmol, 3.30 eq), DIEA (209 g, 1.62 mol, 282 mL, 6.60 eq) and Compound 2C (492 g, 811 mmol, 3.30 eq, TsOH) at 0° C., the mixture is stirred at 15° C. for 16 hrs. LCMS showed the desired MS is given. The mixture is concentrated under vacuum to give a residue, then the mixture is diluted with DCM (2000 mL), washed with 1 N HCl aqueous solution (700 mL*2), then saturated NaHCO₃ aqueous solution (700 mL*2) and concentrated under vacuum. The crude is purified by column chromatography to give Compound 3C (304 g, 155 mmol, 63.1% yield, 96.0% purity) as a yellow solid.

### General Procedure for Preparation of Compound 4C

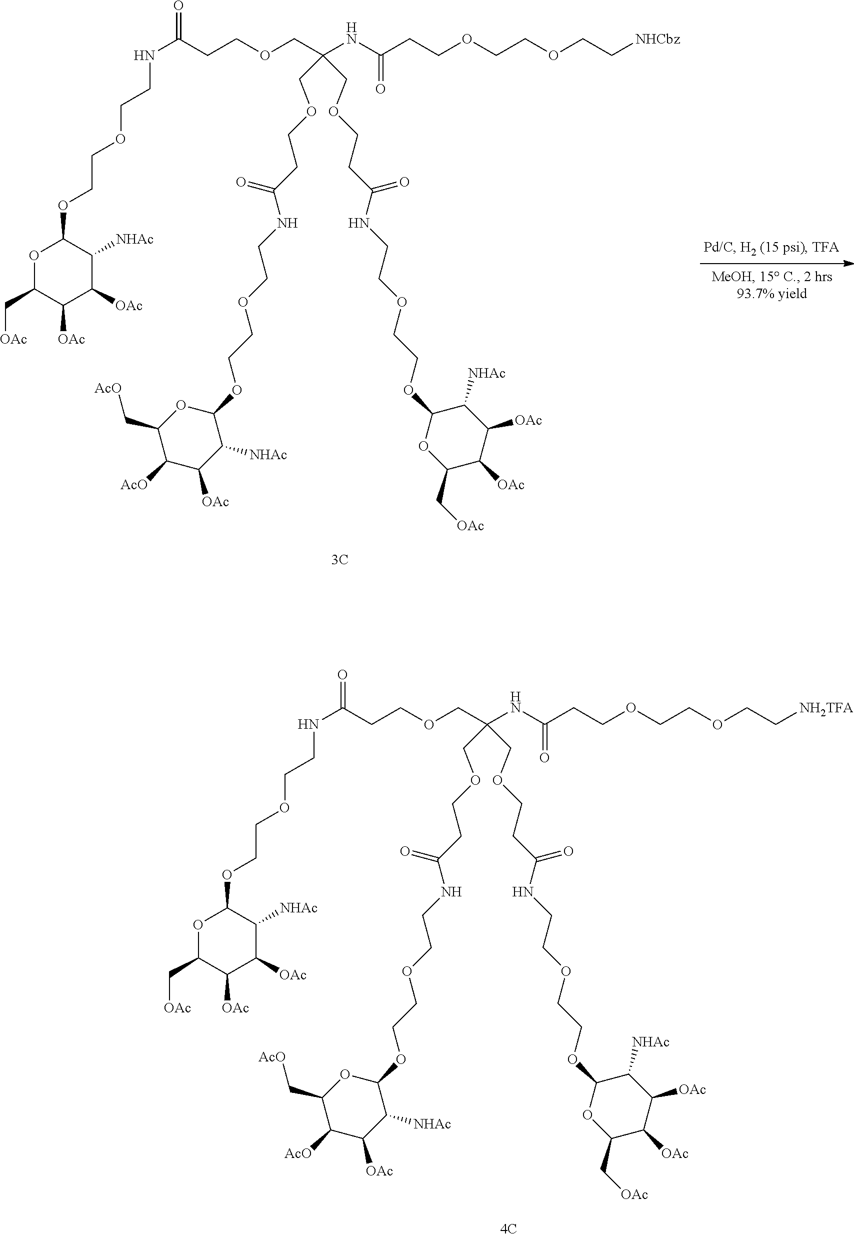

Two batches solution of Compound 3C (55.0 g, 29.2 mmol, 1.00 eq) in MeOH (1600 mL) is added Pd/C (6.60 g, 19.1 mmol, 10.0% purity) and TFA (3.34 g, 29.2 mmol, 2.17 mL, 1.00 eq), the mixture is degassed under vacuum and purged with H₂. The mixture is stirred under H₂ (15 psi) at 15° C. for 2 hours. LCMS showed the desired MS is given. The mixture is filtered and the filtrate is concentrated under vacuum to give Compound 4C (106 g, 54.8 mmol, 93.7% yield, 96.2% purity, TFA) as a white solid.

### General Procedure for Preparation of Compound 5C

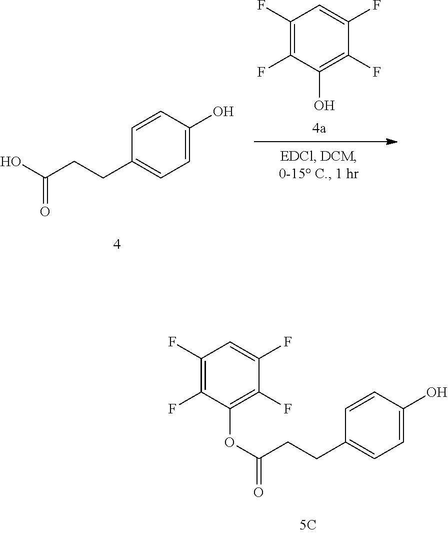 [SMILES: Oc1c(F)c(F)cc(F)c1F ; O=C(O)CCc1ccc(O)cc1 ; O=C(CCc1ccc(O)cc1)Oc1c(F)c(F)cc(F)c1F]

Two batches in parallel. To a solution of EDCI (28.8 g, 150 mmol, 1.00 eq) in DCM (125 mL) is added compound 4a (25.0 g, 150 mmol, 1.00 eq) dropwise at 0° C., then the mixture is added to compound 4 (25.0 g, 150 mmol, 1.00 eq) in DCM (125 mL) at 0° C., then the mixture is stirred at 25° C. for 1 hr. TLC (Petroleum ether:Ethyl acetate=3:1, R_{f}=0.45) showed the reactant is consumed and one new spot is formed. The reaction mixture is diluted with DCM (100 mL) then washed with aq.NaHCO₃ (250 mL*1) and brine (250 mL), dried over Na₂SO₄, filtered and concentrated under reduced pressure to give a residue. The residue is purified by column chromatography (SiO₂, Petroleum ether:Ethyl acetate=100:1 to 3:1), TLC (SiO₂, Petroleum ether:Ethyl acetate=3:1), R_{f}=0.45, then concentrated under reduced pressure to give a residue. Compound 5C (57.0 g, 176 mmol, 58.4% yield, 96.9% purity) is obtained as colorless oil and confirmed ¹HNMR: EW33072-2-P1A, 400 MHz, DMSO 6 9.21 (s, 1H), 7.07-7.09 (m, 2H), 6.67-6.70 (m, 2H), 3.02-3.04 (m, 2H), 2.86-2.90 (m, 2H)

### General Procedure for Preparation of Compound 6

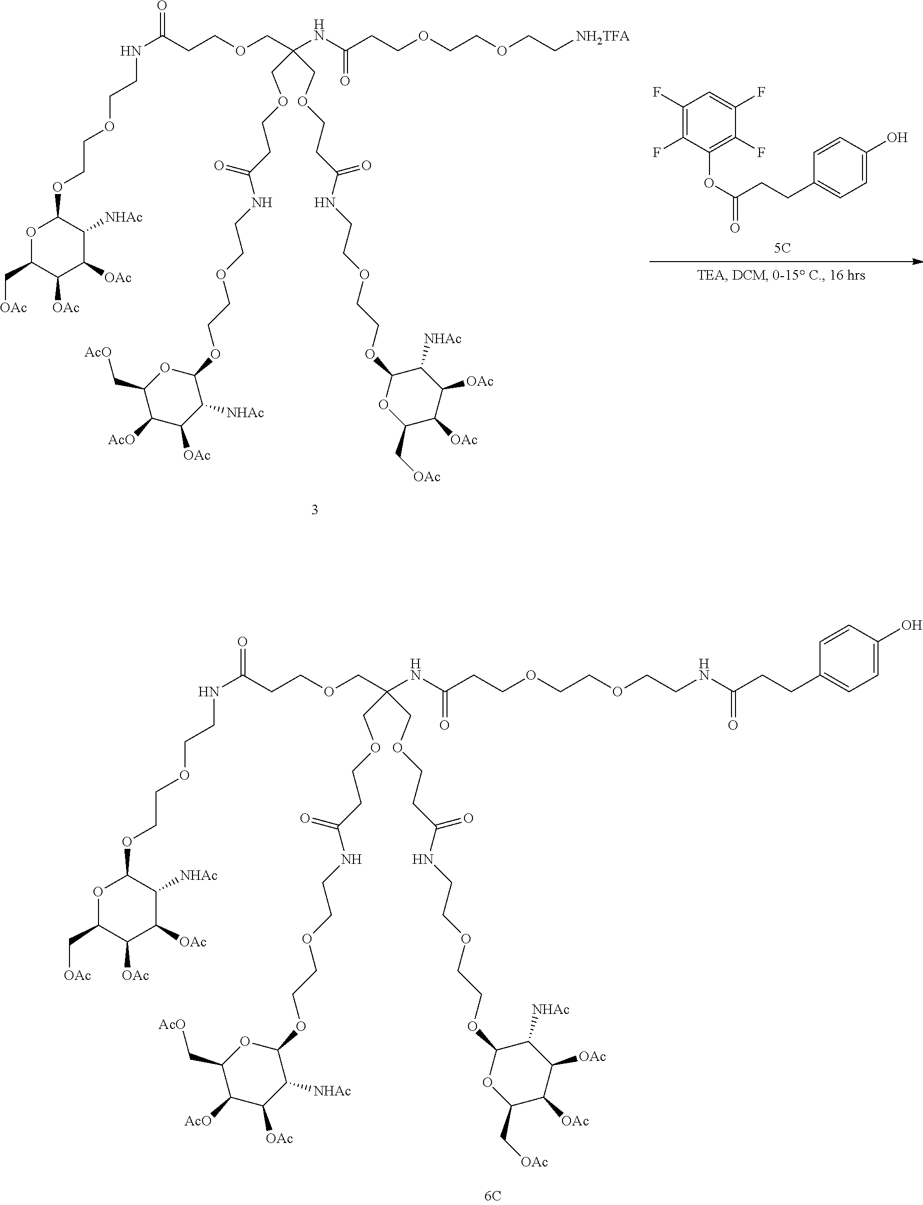

To a mixture of compound 3 (79.0 g, 41.0 mmol, 96.4% purity, 1.00 eq, TFA) and compound 6C (14.2 g, 43.8 mmol, 96.9% purity, 1.07 eq) in DCM (800 mL) is added TEA (16.6 g, 164 mmol, 22.8 mL, 4.00 eq) dropwise at 0° C., the mixture is stirred at 15° C. for 16 hrs. LCMS (EW33072-12-P1B, Rt=0.844 min) showed the desired mass is detected. The reaction mixture is diluted with DCM (400 mL) and washed with aq.NaHCO₃ (400 mL*1) and brine (400 mL*1), then the mixture is diluted with DCM (2.00 L) and washed with 0.7 M Na₂CO₃ (1000 mL*3) and brine (800 mL*3), dried over Na₂SO₄, filtered and concentrated under reduced pressure to give a residue. The residue is used to next step directly without purification. Compound 6 (80.0 g, crude) is obtained as white solid and confirmed via ¹HNMR: EW33072-12-P1A, 400 MHz, MeOD 6 7-2-7.04 (m, 2H), 6.-8-6.70 (m, 2H), 5.-4-5.35 (s, 3H), 5.-7-5.08 (d, J=4.00 Hz, 3H), 4.-2-4.64 (d, J=8.00 Hz, 3H), 3.-1-4.16 (m, 16H), 3.-1-3.70 (m, 44H), 2.-0-2.83 (m, 2H), 2.68 (m, 2H), 2.-6-2.47 (m, 10H), 2.14 (s, 9H), 2.03 (s, 9H), 1.-4-1.95 (d, J=4.00 Hz, 18H).

### General Procedure for Preparation of TriGNal-TRIS-Peg2-Phosph 8c

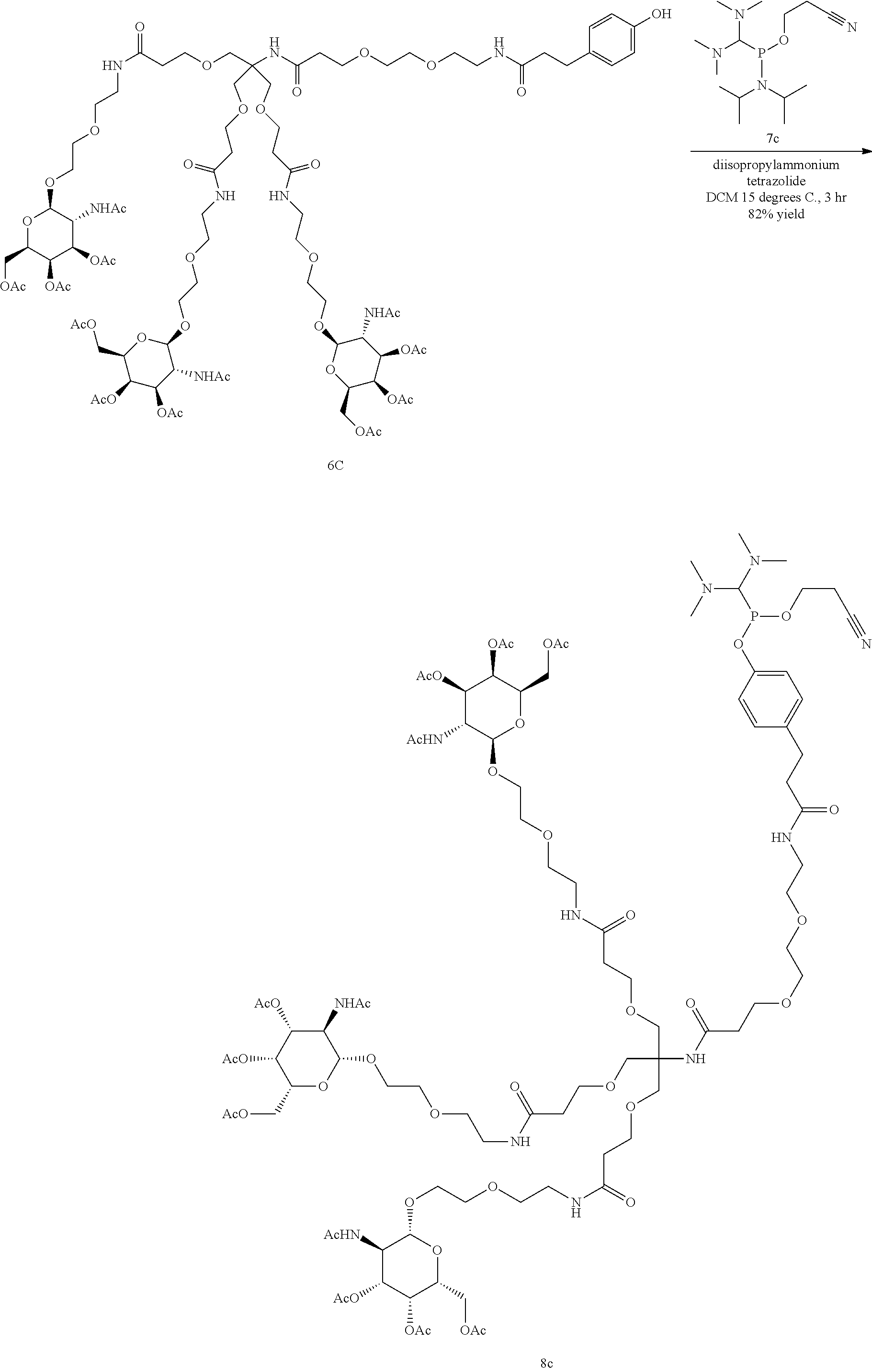

Two batches are synthesized in parallel. To a solution of compound 6C (40.0 g, 21.1 mmol, 1.00 eq in DCM (600 mL) is added diisopropylammonium tetrazolide (3.62 g, 21.1 mmol, 1.00 eq) and compound 7c (6.37 g, 21.1 mmol, 6.71 mL, 1.00 eq) in DCM (8.00 mL) drop-wise, the mixture is stirred at 30° C. for 1 hr, then added compound 7c (3.18 g, 10.6 mmol, 3.35 mL, 0.50 eq) in DCM (8.00 mL) drop-wise, the mixture is stirred at 30° C. for 30 mins, then added compound 7c (3.18 g, 10.6 mmol, 3.35 mL, 0.50 eq) in DCM (8.00 mL) drop-wise, the mixture is stirred at 30° C. for 1.5 hrs. LCMS (EW33072-17-P1C1, Rt=0.921 min) showed the desired MS+1 is detected. LCMS (EW33072-17-P1C2, Rt=0.919 min) showed the desired MS+1 is detected. Two batches are combined for work-up. The mixture is diluted with DCM (1.20 L), washed with saturated NaHCO₃ aqueous solution (1.60 L*2), 3% DMF in H₂O (1.60 L*2), H₂O (1.60 L*3), brine (1.60 L), dried over Na₂SO₄, filtered and concentrated under reduced pressure to give a residue. The residue is purified by column chromatography (SiO₂, DCM:MeOH:TEA=100:3:2) TLC (SiO₂, DCM:MeOH=10:1, R_{f}=0.45), then concentrated under reduced pressure to give a residue. Compound 8C (76.0 g, 34.8 mmol, 82.5% yield, 96.0% purity) is obtained as white solid and confirmed via ¹HNMR: EW33072-19-PIC, 400 MHz, MeOD

δ 7.13-7.15 (d, J=8.50 Hz, 2H), 6.95-6.97 (dd, J=8.38, 1.13 Hz, 2H), 5.34 (d, J=2.88 Hz, 3H), 0.09 (dd, J=11.26, 3.38 Hz, 3H), 4.64 (d, J=8.50 Hz, 3H), 3.-9-4.20 (m, 12H), 3.-8-3.98 (m, 5H), 3.-6-3.83 (m, 20H), 3.-1-3.65 (m, 17H), 3.-3-3.50 (m, 9H), 2.87 (t, J=7.63 Hz, 2H), 2.76 (t, J=5.94 Hz, 2H), 2.-2-2.50 (m, 10H), 2.14 (s, 9H), 2.03 (s, 9H), 1-4-1.95 (d, J=6.13 Hz, 18H), 1.24-1.26 (d, J=6.75 Hz, 6H), 1.18-1.20 (d, J=6.75 Hz, 6H)

### Example 26: Modification Motif 1

An example FGG siRNA includes a combination of the following modifications:
Position 9 (from 5′ to 3′) of the sense strand is 2′F.
If position 9 is a pyrimidine then all purines in the Sense Strand are 2′OMe, and 1-5 pyrimidines between positions 5 and 11 are 2′F provided that there are never three 2′F modifications in a row.
If position 9 is a purine then all pyrimidines in the Sense Strand are 2′OMe, and 1-5 purines between positions 5 and 11 are 2′F provided that there are never three 2′F modifications in a row.
Antisense strand odd-numbered positions are 2′OMe and even-numbered positions are a mixture of 2′F, 2′OMe and 2′deoxy.

### Example 27: Modification Motif 2

An example FGG siRNA includes a combination of the following modifications:
Position 9 (from 5′ to 3′) of the sense strand is 2′deoxy.
Sense strand positions 5, 7 and 8 are 2′F.
All pyrimidines in positions 10-21 are 2′OMe, and purines are a mixture of 2′OMe and 2′F. Alternatively, all purines in positions 10-21 are 2′OMe and all pyrimidines in positions 10-21 are a mixture of 2′OMe and 2′F.
Antisense strand odd-numbered positions are 2′OMe and even-numbered positions are a mixture of 2′F, 2′OMe and 2′deoxy.

### IV. Sequence Information

Some embodiments include one or more nucleic acid sequences in the following tables:

TABLE 78
Sequence information

| SEQ | |
|---|---|
| ID NO: | Description |
| 1-1742 | FGG siRNA sense strand sequences |
| 1743-3484 | FGG siRNA antisense strand sequences |
| 3485-3537 | Modified FGG siRNA sense strand sequences |
| 3538-3590 | Modified FGG siRNA antisense strand sequences |
| 3591-3594 | Modified FGG siRNA sense strand sequences |
| 3595-3598 | Modified FGG siRNA antisense strand sequences |
| 3599-3620 | Modified FGG siRNA sense strand sequences |
| 3621 | Full-length human FGG mRNA sequence (Ensembl Acc. No. |
| | ENST00000404648) |
| 3622-3630 | Modification pattern 1S to 9S |
| 3631-3639 | Modification pattern 1AS to 9AS |
| 3640 | Modification pattern ASO1 |
| 3641-3676 | Modified FGG siRNA sense sequences |
| 3677-3712 | Modified FGG siRNA antisense sequences |
| 3713-3748 | FGG siRNA sense strand sequences |
| 3749-3784 | FGG siRNA antisense sequences |
| 3785-3804 | Modification pattern 10S to 29S |
| 3805-3816 | Modification pattern 10AS to 21AS |

TABLE 79
siRNA sequences

| | SEQ | | SEQ | |
|---|---|---|---|---|
| siRNA | ID | Sense strand sequence | ID | Antisense strand sequence |
| Name | NO: | (5'-3') | NO: | (5'-3') |
| siRNA 1 | 1 | AAAAAGGAGGAGCTTCAAC | 1743 | GTTGAAGCTCCTCCTTTTT |
| siRNA 2 | 2 | AAAAGGAGGAGCTTCAACC | 1744 | GGTTGAAGCTCCTCCTTTT |
| siRNA 3 | 3 | AAAGGAGGAGCTTCAACCT | 1745 | AGGTTGAAGCTCCTCCTTT |
| siRNA 4 | 4 | AAGGAGGAGCTTCAACCTG | 1746 | CAGGTTGAAGCTCCTCCTT |
| siRNA 5 | 5 | AGGAGGAGCTTCAACCTGT | 1747 | ACAGGTTGAAGCTCCTCCT |
| siRNA 6 | 6 | GGAGGAGCTTCAACCTGTG | 1748 | CACAGGTTGAAGCTCCTCC |
| siRNA 7 | 7 | GAGGAGCTTCAACCTGTGT | 1749 | ACACAGGTTGAAGCTCCTC |
| siRNA 8 | 8 | AGGAGCTTCAACCTGTGTG | 1750 | CACACAGGTTGAAGCTCCT |
| siRNA 9 | 9 | GGAGCTTCAACCTGTGTGC | 1751 | GCACACAGGTTGAAGCTCC |
| siRNA 10 | 10 | GAGCTTCAACCTGTGTGCA | 1752 | TGCACACAGGTTGAAGCTC |
| siRNA 11 | 11 | AGCTTCAACCTGTGTGCAA | 1753 | TTGCACACAGGTTGAAGCT |
| siRNA 12 | 12 | GCTTCAACCTGTGTGCAAA | 1754 | TTTGCACACAGGTTGAAGC |
| siRNA 13 | 13 | CTTCAACCTGTGTGCAAAA | 1755 | TTTTGCACACAGGTTGAAG |
| siRNA 14 | 14 | TTCAACCTGTGTGCAAAAT | 1756 | ATTTTGCACACAGGTTGAA |
| siRNA 15 | 15 | TCAACCTGTGTGCAAAATC | 1757 | GATTTTGCACACAGGTTGA |
| siRNA 16 | 16 | CAACCTGTGTGCAAAATCT | 1758 | AGATTTTGCACACAGGTTG |
| siRNA 17 | 17 | AACCTGTGTGCAAAATCTG | 1759 | CAGATTTTGCACACAGGTT |
| siRNA 18 | 18 | ACCTGTGTGCAAAATCTGG | 1760 | CCAGATTTTGCACACAGGT |
| siRNA 19 | 19 | CCTGTGTGCAAAATCTGGG | 1761 | CCCAGATTTTGCACACAGG |
| siRNA 20 | 20 | CTGTGTGCAAAATCTGGGA | 1762 | TCCCAGATTTTGCACACAG |
| siRNA 21 | 21 | TGTGTGCAAAATCTGGGAA | 1763 | TTCCCAGATTTTGCACACA |
| siRNA 22 | 22 | GTGTGCAAAATCTGGGAAC | 1764 | GTTCCCAGATTTTGCACAC |
| siRNA 23 | 23 | TGTGCAAAATCTGGGAACC | 1765 | GGTTCCCAGATTTTGCACA |
| siRNA 24 | 24 | GTGCAAAATCTGGGAACCT | 1766 | AGGTTCCCAGATTTTGCAC |
| siRNA 25 | 25 | TGCAAAATCTGGGAACCTG | 1767 | CAGGTTCCCAGATTTTGCA |
| siRNA 26 | 26 | GCAAAATCTGGGAACCTGA | 1768 | TCAGGTTCCCAGATTTTGC |
| siRNA 27 | 27 | CAAAATCTGGGAACCTGAC | 1769 | GTCAGGTTCCCAGATTTTG |
| siRNA 28 | 28 | AAAATCTGGGAACCTGACA | 1770 | TGTCAGGTTCCCAGATTTT |
| siRNA 29 | 29 | AAATCTGGGAACCTGACAG | 1771 | CTGTCAGGTTCCCAGATTT |
| siRNA 30 | 30 | AATCTGGGAACCTGACAGT | 1772 | ACTGTCAGGTTCCCAGATT |
| siRNA 31 | 31 | ATCTGGGAACCTGACAGTA | 1773 | TACTGTCAGGTTCCCAGAT |
| siRNA 32 | 32 | TCTGGGAACCTGACAGTAT | 1774 | ATACTGTCAGGTTCCCAGA |
| siRNA 33 | 33 | CTGGGAACCTGACAGTATA | 1775 | TATACTGTCAGGTTCCCAG |
| siRNA 34 | 34 | TGGGAACCTGACAGTATAG | 1776 | CTATACTGTCAGGTTCCCA |
| siRNA 35 | 35 | GGGAACCTGACAGTATAGG | 1777 | CCTATACTGTCAGGTTCCC |
| siRNA 36 | 36 | GGAACCTGACAGTATAGGT | 1778 | ACCTATACTGTCAGGTTCC |
| siRNA 37 | 37 | GAACCTGACAGTATAGGTT | 1779 | AACCTATACTGTCAGGTTC |
| siRNA 38 | 38 | AACCTGACAGTATAGGTTG | 1780 | CAACCTATACTGTCAGGTT |
| siRNA 39 | 39 | ACCTGACAGTATAGGTTGG | 1781 | CCAACCTATACTGTCAGGT |
| siRNA 40 | 40 | CCTGACAGTATAGGTTGGG | 1782 | CCCAACCTATACTGTCAGG |
| siRNA 41 | 41 | CTGACAGTATAGGTTGGGG | 1783 | CCCCAACCTATACTGTCAG |
| siRNA 42 | 42 | TGACAGTATAGGTTGGGGG | 1784 | CCCCCAACCTATACTGTCA |
| siRNA 43 | 43 | GACAGTATAGGTTGGGGGC | 1785 | GCCCCCAACCTATACTGTC |
| siRNA 44 | 44 | ACAGTATAGGTTGGGGGCC | 1786 | GGCCCCCAACCTATACTGT |
| siRNA 45 | 45 | CAGTATAGGTTGGGGGCCA | 1787 | TGGCCCCCAACCTATACTG |
| siRNA 46 | 46 | AGTATAGGTTGGGGGCCAG | 1788 | CTGGCCCCCAACCTATACT |
| siRNA 47 | 47 | GTATAGGTTGGGGGCCAGG | 1789 | CCTGGCCCCCAACCTATAC |
| siRNA 48 | 48 | TATAGGTTGGGGGCCAGGA | 1790 | TCCTGGCCCCCAACCTATA |
| siRNA 49 | 49 | ATAGGTTGGGGGCCAGGAT | 1791 | ATCCTGGCCCCCAACCTAT |
| siRNA 50 | 50 | TAGGTTGGGGGCCAGGATG | 1792 | CATCCTGGCCCCCAACCTA |
| siRNA 51 | 51 | AGGTTGGGGGCCAGGATGA | 1793 | TCATCCTGGCCCCCAACCT |
| siRNA 52 | 52 | GGTTGGGGGCCAGGATGAG | 1794 | CTCATCCTGGCCCCCAACC |
| siRNA 53 | 53 | GTTGGGGGCCAGGATGAGG | 1795 | CCTCATCCTGGCCCCCAAC |
| siRNA 54 | 54 | TTGGGGGCCAGGATGAGGA | 1796 | TCCTCATCCTGGCCCCCAA |
| siRNA 55 | 55 | TGGGGGCCAGGATGAGGAA | 1797 | TTCCTCATCCTGGCCCCCA |
| siRNA 56 | 56 | GGGGGCCAGGATGAGGAAA | 1798 | TTTCCTCATCCTGGCCCCC |
| siRNA 57 | 57 | GGGGCCAGGATGAGGAAAA | 1799 | TTTTCCTCATCCTGGCCCC |
| siRNA 58 | 58 | GGGCCAGGATGAGGAAAAA | 1800 | TTTTTCCTCATCCTGGCCC |
| siRNA 59 | 59 | GGCCAGGATGAGGAAAAAG | 1801 | CTTTTTCCTCATCCTGGCC |
| siRNA 60 | 60 | GCCAGGATGAGGAAAAAGG | 1802 | CCTTTTTCCTCATCCTGGC |
| siRNA 61 | 61 | CCAGGATGAGGAAAAAGGA | 1803 | TCCTTTTTCCTCATCCTGG |
| siRNA 62 | 62 | CAGGATGAGGAAAAAGGAA | 1804 | TTCCTTTTTCCTCATCCTG |
| siRNA 63 | 63 | AGGATGAGGAAAAAGGAAC | 1805 | GTTCCTTTTTCCTCATCCT |
| siRNA 64 | 64 | GGATGAGGAAAAAGGAACG | 1806 | CGTTCCTTTTTCCTCATCC |
| siRNA 65 | 65 | GATGAGGAAAAAGGAACGG | 1807 | CCGTTCCTTTTTCCTCATC |
| siRNA 66 | 66 | ATGAGGAAAAAGGAACGGG | 1808 | CCCGTTCCTTTTTCCTCAT |
| siRNA 67 | 67 | TGAGGAAAAAGGAACGGGA | 1809 | TCCCGTTCCTTTTTCCTCA |
| siRNA 68 | 68 | GAGGAAAAAGGAACGGGAA | 1810 | TTCCCGTTCCTTTTTCCTC |
| siRNA 69 | 69 | AGGAAAAAGGAACGGGAAA | 1811 | TTTCCCGTTCCTTTTTCCT |
| siRNA 70 | 70 | GGAAAAAGGAACGGGAAAG | 1812 | CTTTCCCGTTCCTTTTTCC |
| siRNA 71 | 71 | GAAAAAGGAACGGGAAAGA | 1813 | TCTTTCCCGTTCCTTTTTC |
| siRNA 72 | 72 | AAAAAGGAACGGGAAAGAC | 1814 | GTCTTTCCCGTTCCTTTTT |
| siRNA 73 | 73 | AAAAGGAACGGGAAAGACC | 1815 | GGTCTTTCCCGTTCCTTTT |
| siRNA 74 | 74 | AAAGGAACGGGAAAGACCT | 1816 | AGGTCTTTCCCGTTCCTTT |
| siRNA 75 | 75 | AAGGAACGGGAAAGACCTG | 1817 | CAGGTCTTTCCCGTTCCTT |
| siRNA 76 | 76 | AGGAACGGGAAAGACCTGC | 1818 | GCAGGTCTTTCCCGTTCCT |
| siRNA 77 | 77 | GGAACGGGAAAGACCTGCC | 1819 | GGCAGGTCTTTCCCGTTCC |
| siRNA 78 | 78 | GAACGGGAAAGACCTGCCC | 1820 | GGGCAGGTCTTTCCCGTTC |
| siRNA 79 | 79 | AACGGGAAAGACCTGCCCA | 1821 | TGGGCAGGTCTTTCCCGTT |
| siRNA 80 | 80 | ACGGGAAAGACCTGCCCAC | 1822 | GTGGGCAGGTCTTTCCCGT |
| siRNA 81 | 81 | CGGGAAAGACCTGCCCACC | 1823 | GGTGGGCAGGTCTTTCCCG |
| siRNA 82 | 82 | GGGAAAGACCTGCCCACCC | 1824 | GGGTGGGCAGGTCTTTCCC |
| siRNA 83 | 83 | GGAAAGACCTGCCCACCCT | 1825 | AGGGTGGGCAGGTCTTTCC |
| siRNA 84 | 84 | GAAAGACCTGCCCACCCTT | 1826 | AAGGGTGGGCAGGTCTTTC |
| siRNA 85 | 85 | AAAGACCTGCCCACCCTTC | 1827 | GAAGGGTGGGCAGGTCTTT |
| siRNA 86 | 86 | AAGACCTGCCCACCCTTCT | 1828 | AGAAGGGTGGGCAGGTCTT |
| siRNA 87 | 87 | AGACCTGCCCACCCTTCTG | 1829 | CAGAAGGGTGGGCAGGTCT |
| siRNA 88 | 88 | GACCTGCCCACCCTTCTGG | 1830 | CCAGAAGGGTGGGCAGGTC |
| siRNA 89 | 89 | ACCTGCCCACCCTTCTGGT | 1831 | ACCAGAAGGGTGGGCAGGT |
| siRNA 90 | 90 | CCTGCCCACCCTTCTGGTA | 1832 | TACCAGAAGGGTGGGCAGG |
| siRNA 91 | 91 | CTGCCCACCCTTCTGGTAA | 1833 | TTACCAGAAGGGTGGGCAG |
| siRNA 92 | 92 | TGCCCACCCTTCTGGTAAG | 1834 | CTTACCAGAAGGGTGGGCA |
| siRNA 93 | 93 | GCCCACCCTTCTGGTAAGG | 1835 | CCTTACCAGAAGGGTGGGC |
| siRNA 94 | 94 | CCCACCCTTCTGGTAAGGA | 1836 | TCCTTACCAGAAGGGTGGG |
| siRNA 95 | 95 | CCACCCTTCTGGTAAGGAG | 1837 | CTCCTTACCAGAAGGGTGG |
| siRNA 96 | 96 | CACCCTTCTGGTAAGGAGG | 1838 | CCTCCTTACCAGAAGGGTG |
| siRNA 97 | 97 | ACCCTTCTGGTAAGGAGGC | 1839 | GCCTCCTTACCAGAAGGGT |
| siRNA 98 | 98 | CCCTTCTGGTAAGGAGGCC | 1840 | GGCCTCCTTACCAGAAGGG |
| siRNA 99 | 99 | CCTTCTGGTAAGGAGGCCC | 1841 | GGGCCTCCTTACCAGAAGG |
| siRNA 100 | 100 | CTTCTGGTAAGGAGGCCCC | 1842 | GGGGCCTCCTTACCAGAAG |
| siRNA 101 | 101 | TTCTGGTAAGGAGGCCCCG | 1843 | CGGGGCCTCCTTACCAGAA |
| siRNA 102 | 102 | TCTGGTAAGGAGGCCCCGT | 1844 | ACGGGGCCTCCTTACCAGA |
| siRNA 103 | 103 | CTGGTAAGGAGGCCCCGTG | 1845 | CACGGGGCCTCCTTACCAG |
| siRNA 104 | 104 | TGGTAAGGAGGCCCCGTGA | 1846 | TCACGGGGCCTCCTTACCA |
| siRNA 105 | 105 | GGTAAGGAGGCCCCGTGAT | 1847 | ATCACGGGGCCTCCTTACC |
| siRNA 106 | 106 | GTAAGGAGGCCCCGTGATC | 1848 | GATCACGGGGCCTCCTTAC |
| siRNA 107 | 107 | TAAGGAGGCCCCGTGATCA | 1849 | TGATCACGGGGCCTCCTTA |
| siRNA 108 | 108 | AAGGAGGCCCCGTGATCAG | 1850 | CTGATCACGGGGCCTCCTT |
| siRNA 109 | 109 | AGGAGGCCCCGTGATCAGC | 1851 | GCTGATCACGGGGCCTCCT |
| siRNA 110 | 110 | GGAGGCCCCGTGATCAGCT | 1852 | AGCTGATCACGGGGCCTCC |
| siRNA 111 | 111 | GAGGCCCCGTGATCAGCTC | 1853 | GAGCTGATCACGGGGCCTC |
| siRNA 112 | 112 | AGGCCCCGTGATCAGCTCC | 1854 | GGAGCTGATCACGGGGCCT |
| siRNA 113 | 113 | GGCCCCGTGATCAGCTCCA | 1855 | TGGAGCTGATCACGGGGCC |
| siRNA 114 | 114 | GCCCCGTGATCAGCTCCAG | 1856 | CTGGAGCTGATCACGGGGC |
| siRNA 115 | 115 | CCCCGTGATCAGCTCCAGC | 1857 | GCTGGAGCTGATCACGGGG |
| siRNA 116 | 116 | CCCGTGATCAGCTCCAGCC | 1858 | GGCTGGAGCTGATCACGGG |
| siRNA 117 | 117 | CCGTGATCAGCTCCAGCCA | 1859 | TGGCTGGAGCTGATCACGG |
| siRNA 118 | 118 | CGTGATCAGCTCCAGCCAT | 1860 | ATGGCTGGAGCTGATCACG |
| siRNA 119 | 119 | GTGATCAGCTCCAGCCATT | 1861 | AATGGCTGGAGCTGATCAC |
| siRNA 120 | 120 | TGATCAGCTCCAGCCATTT | 1862 | AAATGGCTGGAGCTGATCA |
| siRNA 121 | 121 | GATCAGCTCCAGCCATTTG | 1863 | CAAATGGCTGGAGCTGATC |
| siRNA 122 | 122 | ATCAGCTCCAGCCATTTGC | 1864 | GCAAATGGCTGGAGCTGAT |
| siRNA 123 | 123 | TCAGCTCCAGCCATTTGCA | 1865 | TGCAAATGGCTGGAGCTGA |
| siRNA 124 | 124 | CAGCTCCAGCCATTTGCAG | 1866 | CTGCAAATGGCTGGAGCTG |
| siRNA 125 | 125 | AGCTCCAGCCATTTGCAGT | 1867 | ACTGCAAATGGCTGGAGCT |
| siRNA 126 | 126 | GCTCCAGCCATTTGCAGTC | 1868 | GACTGCAAATGGCTGGAGC |
| siRNA 127 | 127 | CTCCAGCCATTTGCAGTCC | 1869 | GGACTGCAAATGGCTGGAG |
| siRNA 128 | 128 | TCCAGCCATTTGCAGTCCT | 1870 | AGGACTGCAAATGGCTGGA |
| siRNA 129 | 129 | CCAGCCATTTGCAGTCCTG | 1871 | CAGGACTGCAAATGGCTGG |
| siRNA 130 | 130 | CAGCCATTTGCAGTCCTGG | 1872 | CCAGGACTGCAAATGGCTG |
| siRNA 131 | 131 | AGCCATTTGCAGTCCTGGC | 1873 | GCCAGGACTGCAAATGGCT |
| siRNA 132 | 132 | GCCATTTGCAGTCCTGGCT | 1874 | AGCCAGGACTGCAAATGGC |
| siRNA 133 | 133 | CCATTTGCAGTCCTGGCTA | 1875 | TAGCCAGGACTGCAAATGG |
| siRNA 134 | 134 | CATTTGCAGTCCTGGCTAT | 1876 | ATAGCCAGGACTGCAAATG |
| siRNA 135 | 135 | ATTTGCAGTCCTGGCTATC | 1877 | GATAGCCAGGACTGCAAAT |
| siRNA 136 | 136 | TTTGCAGTCCTGGCTATCC | 1878 | GGATAGCCAGGACTGCAAA |
| siRNA 137 | 137 | TTGCAGTCCTGGCTATCCC | 1879 | GGGATAGCCAGGACTGCAA |
| siRNA 138 | 138 | TGCAGTCCTGGCTATCCCA | 1880 | TGGGATAGCCAGGACTGCA |
| siRNA 139 | 139 | GCAGTCCTGGCTATCCCAG | 1881 | CTGGGATAGCCAGGACTGC |
| siRNA 140 | 140 | CAGTCCTGGCTATCCCAGG | 1882 | CCTGGGATAGCCAGGACTG |
| siRNA 141 | 141 | AGTCCTGGCTATCCCAGGA | 1883 | TCCTGGGATAGCCAGGACT |
| siRNA 142 | 142 | GTCCTGGCTATCCCAGGAG | 1884 | CTCCTGGGATAGCCAGGAC |
| siRNA 143 | 143 | TCCTGGCTATCCCAGGAGC | 1885 | GCTCCTGGGATAGCCAGGA |
| siRNA 144 | 144 | CCTGGCTATCCCAGGAGCT | 1886 | AGCTCCTGGGATAGCCAGG |
| siRNA 145 | 145 | CTGGCTATCCCAGGAGCTT | 1887 | AAGCTCCTGGGATAGCCAG |
| siRNA 146 | 146 | TGGCTATCCCAGGAGCTTA | 1888 | TAAGCTCCTGGGATAGCCA |
| siRNA 147 | 147 | GGCTATCCCAGGAGCTTAC | 1889 | GTAAGCTCCTGGGATAGCC |
| siRNA 148 | 148 | GCTATCCCAGGAGCTTACA | 1890 | TGTAAGCTCCTGGGATAGC |
| siRNA 149 | 149 | CTATCCCAGGAGCTTACAT | 1891 | ATGTAAGCTCCTGGGATAG |
| siRNA 150 | 150 | TATCCCAGGAGCTTACATA | 1892 | TATGTAAGCTCCTGGGATA |
| siRNA 151 | 151 | ATCCCAGGAGCTTACATAA | 1893 | TTATGTAAGCTCCTGGGAT |
| siRNA 152 | 152 | TCCCAGGAGCTTACATAAA | 1894 | TTTATGTAAGCTCCTGGGA |
| siRNA 153 | 153 | CCCAGGAGCTTACATAAAG | 1895 | CTTTATGTAAGCTCCTGGG |
| siRNA 154 | 154 | CCAGGAGCTTACATAAAGG | 1896 | CCTTTATGTAAGCTCCTGG |
| siRNA 155 | 155 | CAGGAGCTTACATAAAGGG | 1897 | CCCTTTATGTAAGCTCCTG |
| siRNA 156 | 156 | AGGAGCTTACATAAAGGGA | 1898 | TCCCTTTATGTAAGCTCCT |
| siRNA 157 | 157 | GGAGCTTACATAAAGGGAC | 1899 | GTCCCTTTATGTAAGCTCC |
| siRNA 158 | 158 | GAGCTTACATAAAGGGACA | 1900 | TGTCCCTTTATGTAAGCTC |
| siRNA 159 | 159 | AGCTTACATAAAGGGACAA | 1901 | TTGTCCCTTTATGTAAGCT |
| siRNA 160 | 160 | GCTTACATAAAGGGACAAT | 1902 | ATTGTCCCTTTATGTAAGC |
| siRNA 161 | 161 | CTTACATAAAGGGACAATT | 1903 | AATTGTCCCTTTATGTAAG |
| siRNA 162 | 162 | TTACATAAAGGGACAATTG | 1904 | CAATTGTCCCTTTATGTAA |
| siRNA 163 | 163 | TACATAAAGGGACAATTGG | 1905 | CCAATTGTCCCTTTATGTA |
| siRNA 164 | 164 | ACATAAAGGGACAATTGGA | 1906 | TCCAATTGTCCCTTTATGT |
| siRNA 165 | 165 | CATAAAGGGACAATTGGAG | 1907 | CTCCAATTGTCCCTTTATG |
| siRNA 166 | 166 | ATAAAGGGACAATTGGAGC | 1908 | GCTCCAATTGTCCCTTTAT |
| siRNA 167 | 167 | TAAAGGGACAATTGGAGCC | 1909 | GGCTCCAATTGTCCCTTTA |
| siRNA 168 | 168 | AAAGGGACAATTGGAGCCT | 1910 | AGGCTCCAATTGTCCCTTT |
| siRNA 169 | 169 | AAGGGACAATTGGAGCCTG | 1911 | CAGGCTCCAATTGTCCCTT |
| siRNA 170 | 170 | AGGGACAATTGGAGCCTGA | 1912 | TCAGGCTCCAATTGTCCCT |
| siRNA 171 | 171 | GGGACAATTGGAGCCTGAG | 1913 | CTCAGGCTCCAATTGTCCC |
| siRNA 172 | 172 | GGACAATTGGAGCCTGAGA | 1914 | TCTCAGGCTCCAATTGTCC |
| siRNA 173 | 173 | GACAATTGGAGCCTGAGAG | 1915 | CTCTCAGGCTCCAATTGTC |
| siRNA 174 | 174 | ACAATTGGAGCCTGAGAGG | 1916 | CCTCTCAGGCTCCAATTGT |
| siRNA 175 | 175 | CAATTGGAGCCTGAGAGGT | 1917 | ACCTCTCAGGCTCCAATTG |
| siRNA 176 | 176 | AATTGGAGCCTGAGAGGTG | 1918 | CACCTCTCAGGCTCCAATT |
| siRNA 177 | 177 | ATTGGAGCCTGAGAGGTGA | 1919 | TCACCTCTCAGGCTCCAAT |
| siRNA 178 | 178 | TTGGAGCCTGAGAGGTGAC | 1920 | GTCACCTCTCAGGCTCCAA |
| siRNA 179 | 179 | TGGAGCCTGAGAGGTGACA | 1921 | TGTCACCTCTCAGGCTCCA |
| siRNA 180 | 180 | GGAGCCTGAGAGGTGACAG | 1922 | CTGTCACCTCTCAGGCTCC |
| siRNA 181 | 181 | GAGCCTGAGAGGTGACAGT | 1923 | ACTGTCACCTCTCAGGCTC |
| siRNA 182 | 182 | AGCCTGAGAGGTGACAGTG | 1924 | CACTGTCACCTCTCAGGCT |
| siRNA 183 | 183 | GCCTGAGAGGTGACAGTGC | 1925 | GCACTGTCACCTCTCAGGC |
| siRNA 184 | 184 | CCTGAGAGGTGACAGTGCT | 1926 | AGCACTGTCACCTCTCAGG |
| siRNA 185 | 185 | CTGAGAGGTGACAGTGCTG | 1927 | CAGCACTGTCACCTCTCAG |
| siRNA 186 | 186 | TGAGAGGTGACAGTGCTGA | 1928 | TCAGCACTGTCACCTCTCA |
| siRNA 187 | 187 | GAGAGGTGACAGTGCTGAC | 1929 | GTCAGCACTGTCACCTCTC |
| siRNA 188 | 188 | AGAGGTGACAGTGCTGACA | 1930 | TGTCAGCACTGTCACCTCT |
| siRNA 189 | 189 | GAGGTGACAGTGCTGACAC | 1931 | GTGTCAGCACTGTCACCTC |
| siRNA 190 | 190 | AGGTGACAGTGCTGACACT | 1932 | AGTGTCAGCACTGTCACCT |
| siRNA 191 | 191 | GGTGACAGTGCTGACACTA | 1933 | TAGTGTCAGCACTGTCACC |
| siRNA 192 | 192 | GTGACAGTGCTGACACTAC | 1934 | GTAGTGTCAGCACTGTCAC |
| siRNA 193 | 193 | TGACAGTGCTGACACTACA | 1935 | TGTAGTGTCAGCACTGTCA |
| siRNA 194 | 194 | GACAGTGCTGACACTACAA | 1936 | TTGTAGTGTCAGCACTGTC |
| siRNA 195 | 195 | ACAGTGCTGACACTACAAG | 1937 | CTTGTAGTGTCAGCACTGT |
| siRNA 196 | 196 | CAGTGCTGACACTACAAGG | 1938 | CCTTGTAGTGTCAGCACTG |
| siRNA 197 | 197 | AGTGCTGACACTACAAGGC | 1939 | GCCTTGTAGTGTCAGCACT |
| siRNA 198 | 198 | GTGCTGACACTACAAGGCT | 1940 | AGCCTTGTAGTGTCAGCAC |
| siRNA 199 | 199 | TGCTGACACTACAAGGCTC | 1941 | GAGCCTTGTAGTGTCAGCA |
| siRNA 200 | 200 | GCTGACACTACAAGGCTCG | 1942 | CGAGCCTTGTAGTGTCAGC |
| siRNA 201 | 201 | CTGACACTACAAGGCTCGG | 1943 | CCGAGCCTTGTAGTGTCAG |
| siRNA 202 | 202 | TGACACTACAAGGCTCGGA | 1944 | TCCGAGCCTTGTAGTGTCA |
| siRNA 203 | 203 | GACACTACAAGGCTCGGAG | 1945 | CTCCGAGCCTTGTAGTGTC |
| siRNA 204 | 204 | ACACTACAAGGCTCGGAGC | 1946 | GCTCCGAGCCTTGTAGTGT |
| siRNA 205 | 205 | CACTACAAGGCTCGGAGCT | 1947 | AGCTCCGAGCCTTGTAGTG |
| siRNA 206 | 206 | ACTACAAGGCTCGGAGCTC | 1948 | GAGCTCCGAGCCTTGTAGT |
| siRNA 207 | 207 | CTACAAGGCTCGGAGCTCC | 1949 | GGAGCTCCGAGCCTTGTAG |
| siRNA 208 | 208 | TACAAGGCTCGGAGCTCCG | 1950 | CGGAGCTCCGAGCCTTGTA |
| siRNA 209 | 209 | ACAAGGCTCGGAGCTCCGG | 1951 | CCGGAGCTCCGAGCCTTGT |
| siRNA 210 | 210 | CAAGGCTCGGAGCTCCGGG | 1952 | CCCGGAGCTCCGAGCCTTG |
| siRNA 211 | 211 | AAGGCTCGGAGCTCCGGGC | 1953 | GCCCGGAGCTCCGAGCCTT |
| siRNA 212 | 212 | AGGCTCGGAGCTCCGGGCA | 1954 | TGCCCGGAGCTCCGAGCCT |
| siRNA 213 | 213 | GGCTCGGAGCTCCGGGCAC | 1955 | GTGCCCGGAGCTCCGAGCC |
| siRNA 214 | 214 | GCTCGGAGCTCCGGGCACT | 1956 | AGTGCCCGGAGCTCCGAGC |
| siRNA 215 | 215 | CTCGGAGCTCCGGGCACTC | 1957 | GAGTGCCCGGAGCTCCGAG |
| siRNA 216 | 216 | TCGGAGCTCCGGGCACTCA | 1958 | TGAGTGCCCGGAGCTCCGA |
| siRNA 217 | 217 | CGGAGCTCCGGGCACTCAG | 1959 | CTGAGTGCCCGGAGCTCCG |
| siRNA 218 | 218 | GGAGCTCCGGGCACTCAGA | 1960 | TCTGAGTGCCCGGAGCTCC |
| siRNA 219 | 219 | GAGCTCCGGGCACTCAGAC | 1961 | GTCTGAGTGCCCGGAGCTC |
| siRNA 220 | 220 | AGCTCCGGGCACTCAGACA | 1962 | TGTCTGAGTGCCCGGAGCT |
| siRNA 221 | 221 | GCTCCGGGCACTCAGACAT | 1963 | ATGTCTGAGTGCCCGGAGC |
| siRNA 222 | 222 | CTCCGGGCACTCAGACATC | 1964 | GATGTCTGAGTGCCCGGAG |
| siRNA 223 | 223 | TCCGGGCACTCAGACATCA | 1965 | TGATGTCTGAGTGCCCGGA |
| siRNA 224 | 224 | CCGGGCACTCAGACATCAT | 1966 | ATGATGTCTGAGTGCCCGG |
| siRNA 225 | 225 | CGGGCACTCAGACATCATG | 1967 | CATGATGTCTGAGTGCCCG |
| siRNA 226 | 226 | GGGCACTCAGACATCATGA | 1968 | TCATGATGTCTGAGTGCCC |
| siRNA 227 | 227 | GGCACTCAGACATCATGAG | 1969 | CTCATGATGTCTGAGTGCC |
| siRNA 228 | 228 | GCACTCAGACATCATGAGT | 1970 | ACTCATGATGTCTGAGTGC |
| siRNA 229 | 229 | CACTCAGACATCATGAGTT | 1971 | AACTCATGATGTCTGAGTG |
| siRNA 230 | 230 | ACTCAGACATCATGAGTTG | 1972 | CAACTCATGATGTCTGAGT |
| siRNA 231 | 231 | CTCAGACATCATGAGTTGG | 1973 | CCAACTCATGATGTCTGAG |
| siRNA 232 | 232 | TCAGACATCATGAGTTGGT | 1974 | ACCAACTCATGATGTCTGA |
| siRNA 233 | 233 | CAGACATCATGAGTTGGTC | 1975 | GACCAACTCATGATGTCTG |
| siRNA 234 | 234 | AGACATCATGAGTTGGTCC | 1976 | GGACCAACTCATGATGTCT |
| siRNA 235 | 235 | GACATCATGAGTTGGTCCT | 1977 | AGGACCAACTCATGATGTC |
| siRNA 236 | 236 | ACATCATGAGTTGGTCCTT | 1978 | AAGGACCAACTCATGATGT |
| siRNA 237 | 237 | CATCATGAGTTGGTCCTTG | 1979 | CAAGGACCAACTCATGATG |
| siRNA 238 | 238 | ATCATGAGTTGGTCCTTGC | 1980 | GCAAGGACCAACTCATGAT |
| siRNA 239 | 239 | TCATGAGTTGGTCCTTGCA | 1981 | TGCAAGGACCAACTCATGA |
| siRNA 240 | 240 | CATGAGTTGGTCCTTGCAC | 1982 | GTGCAAGGACCAACTCATG |
| siRNA 241 | 241 | ATGAGTTGGTCCTTGCACC | 1983 | GGTGCAAGGACCAACTCAT |
| siRNA 242 | 242 | TGAGTTGGTCCTTGCACCC | 1984 | GGGTGCAAGGACCAACTCA |
| siRNA 243 | 243 | GAGTTGGTCCTTGCACCCC | 1985 | GGGGTGCAAGGACCAACTC |
| siRNA 244 | 244 | AGTTGGTCCTTGCACCCCC | 1986 | GGGGGTGCAAGGACCAACT |
| siRNA 245 | 245 | GTTGGTCCTTGCACCCCCG | 1987 | CGGGGGTGCAAGGACCAAC |
| siRNA 246 | 246 | TTGGTCCTTGCACCCCCGG | 1988 | CCGGGGGTGCAAGGACCAA |
| siRNA 247 | 247 | TGGTCCTTGCACCCCCGGA | 1989 | TCCGGGGGTGCAAGGACCA |
| siRNA 248 | 248 | GGTCCTTGCACCCCCGGAA | 1990 | TTCCGGGGGTGCAAGGACC |
| siRNA 249 | 249 | GTCCTTGCACCCCCGGAAT | 1991 | ATTCCGGGGGTGCAAGGAC |
| siRNA 250 | 250 | TCCTTGCACCCCCGGAATT | 1992 | AATTCCGGGGGTGCAAGGA |
| siRNA 251 | 251 | CCTTGCACCCCCGGAATTT | 1993 | AAATTCCGGGGGTGCAAGG |
| siRNA 252 | 252 | CTTGCACCCCCGGAATTTA | 1994 | TAAATTCCGGGGGTGCAAG |
| siRNA 253 | 253 | TTGCACCCCCGGAATTTAA | 1995 | TTAAATTCCGGGGGTGCAA |
| siRNA 254 | 254 | TGCACCCCCGGAATTTAAT | 1996 | ATTAAATTCCGGGGGTGCA |
| siRNA 255 | 255 | GCACCCCCGGAATTTAATT | 1997 | AATTAAATTCCGGGGGTGC |
| siRNA 256 | 256 | CACCCCCGGAATTTAATTC | 1998 | GAATTAAATTCCGGGGGTG |
| siRNA 257 | 257 | ACCCCCGGAATTTAATTCT | 1999 | AGAATTAAATTCCGGGGGT |
| siRNA 258 | 258 | CCCCCGGAATTTAATTCTC | 2000 | GAGAATTAAATTCCGGGGG |
| siRNA 259 | 259 | CCCCGGAATTTAATTCTCT | 2001 | AGAGAATTAAATTCCGGGG |
| siRNA 260 | 260 | CCCGGAATTTAATTCTCTA | 2002 | TAGAGAATTAAATTCCGGG |
| siRNA 261 | 261 | CCGGAATTTAATTCTCTAC | 2003 | GTAGAGAATTAAATTCCGG |
| siRNA 262 | 262 | CGGAATTTAATTCTCTACT | 2004 | AGTAGAGAATTAAATTCCG |
| siRNA 263 | 263 | GGAATTTAATTCTCTACTT | 2005 | AAGTAGAGAATTAAATTCC |
| siRNA 264 | 264 | GAATTTAATTCTCTACTTC | 2006 | GAAGTAGAGAATTAAATTC |
| siRNA 265 | 265 | AATTTAATTCTCTACTTCT | 2007 | AGAAGTAGAGAATTAAATT |
| siRNA 266 | 266 | ATTTAATTCTCTACTTCTA | 2008 | TAGAAGTAGAGAATTAAAT |
| siRNA 267 | 267 | TTTAATTCTCTACTTCTAT | 2009 | ATAGAAGTAGAGAATTAAA |
| siRNA 268 | 268 | TTAATTCTCTACTTCTATG | 2010 | CATAGAAGTAGAGAATTAA |
| siRNA 269 | 269 | TAATTCTCTACTTCTATGC | 2011 | GCATAGAAGTAGAGAATTA |
| siRNA 270 | 270 | AATTCTCTACTTCTATGCT | 2012 | AGCATAGAAGTAGAGAATT |
| siRNA 271 | 271 | ATTCTCTACTTCTATGCTC | 2013 | GAGCATAGAAGTAGAGAAT |
| siRNA 272 | 272 | TTCTCTACTTCTATGCTCT | 2014 | AGAGCATAGAAGTAGAGAA |
| siRNA 273 | 273 | TCTCTACTTCTATGCTCTT | 2015 | AAGAGCATAGAAGTAGAGA |
| siRNA 274 | 274 | CTCTACTTCTATGCTCTTT | 2016 | AAAGAGCATAGAAGTAGAG |
| siRNA 275 | 275 | TCTACTTCTATGCTCTTTT | 2017 | AAAAGAGCATAGAAGTAGA |
| siRNA 276 | 276 | CTACTTCTATGCTCTTTTA | 2018 | TAAAAGAGCATAGAAGTAG |
| siRNA 277 | 277 | TACTTCTATGCTCTTTTAT | 2019 | ATAAAAGAGCATAGAAGTA |
| siRNA 278 | 278 | ACTTCTATGCTCTTTTATT | 2020 | AATAAAAGAGCATAGAAGT |
| siRNA 279 | 279 | CTTCTATGCTCTTTTATTT | 2021 | AAATAAAAGAGCATAGAAG |
| siRNA 280 | 280 | TTCTATGCTCTTTTATTTC | 2022 | GAAATAAAAGAGCATAGAA |
| siRNA 281 | 281 | TCTATGCTCTTTTATTTCT | 2023 | AGAAATAAAAGAGCATAGA |
| siRNA 282 | 282 | CTATGCTCTTTTATTTCTC | 2024 | GAGAAATAAAAGAGCATAG |
| siRNA 283 | 283 | TATGCTCTTTTATTTCTCT | 2025 | AGAGAAATAAAAGAGCATA |
| siRNA 284 | 284 | ATGCTCTTTTATTTCTCTC | 2026 | GAGAGAAATAAAAGAGCAT |
| siRNA 285 | 285 | TGCTCTTTTATTTCTCTCT | 2027 | AGAGAGAAATAAAAGAGCA |
| siRNA 286 | 286 | GCTCTTTTATTTCTCTCTT | 2028 | AAGAGAGAAATAAAAGAGC |
| siRNA 287 | 287 | CTCTTTTATTTCTCTCTTC | 2029 | GAAGAGAGAAATAAAAGAG |
| siRNA 288 | 288 | TCTTTTATTTCTCTCTTCA | 2030 | TGAAGAGAGAAATAAAAGA |
| siRNA 289 | 289 | CTTTTATTTCTCTCTTCAA | 2031 | TTGAAGAGAGAAATAAAAG |
| siRNA 290 | 290 | TTTTATTTCTCTCTTCAAC | 2032 | GTTGAAGAGAGAAATAAAA |
| siRNA 291 | 291 | TTTATTTCTCTCTTCAACA | 2033 | TGTTGAAGAGAGAAATAAA |
| siRNA 292 | 292 | TTATTTCTCTCTTCAACAT | 2034 | ATGTTGAAGAGAGAAATAA |
| siRNA 293 | 293 | TATTTCTCTCTTCAACATG | 2035 | CATGTTGAAGAGAGAAATA |
| siRNA 294 | 294 | ATTTCTCTCTTCAACATGT | 2036 | ACATGTTGAAGAGAGAAAT |
| siRNA 295 | 295 | TTTCTCTCTTCAACATGTG | 2037 | CACATGTTGAAGAGAGAAA |
| siRNA 296 | 296 | TTCTCTCTTCAACATGTGT | 2038 | ACACATGTTGAAGAGAGAA |
| siRNA 297 | 297 | TCTCTCTTCAACATGTGTA | 2039 | TACACATGTTGAAGAGAGA |
| siRNA 298 | 298 | CTCTCTTCAACATGTGTAG | 2040 | CTACACATGTTGAAGAGAG |
| siRNA 299 | 299 | TCTCTTCAACATGTGTAGC | 2041 | GCTACACATGTTGAAGAGA |
| siRNA 300 | 300 | CTCTTCAACATGTGTAGCA | 2042 | TGCTACACATGTTGAAGAG |
| siRNA 301 | 301 | TCTTCAACATGTGTAGCAT | 2043 | ATGCTACACATGTTGAAGA |
| siRNA 302 | 302 | CTTCAACATGTGTAGCATA | 2044 | TATGCTACACATGTTGAAG |
| siRNA 303 | 303 | TTCAACATGTGTAGCATAT | 2045 | ATATGCTACACATGTTGAA |
| siRNA 304 | 304 | TCAACATGTGTAGCATATG | 2046 | CATATGCTACACATGTTGA |
| siRNA 305 | 305 | CAACATGTGTAGCATATGT | 2047 | ACATATGCTACACATGTTG |
| siRNA 306 | 306 | AACATGTGTAGCATATGTT | 2048 | AACATATGCTACACATGTT |
| siRNA 307 | 307 | ACATGTGTAGCATATGTTG | 2049 | CAACATATGCTACACATGT |
| siRNA 308 | 308 | CATGTGTAGCATATGTTGC | 2050 | GCAACATATGCTACACATG |
| siRNA 309 | 309 | ATGTGTAGCATATGTTGCT | 2051 | AGCAACATATGCTACACAT |
| siRNA 310 | 310 | TGTGTAGCATATGTTGCTA | 2052 | TAGCAACATATGCTACACA |
| siRNA 311 | 311 | GTGTAGCATATGTTGCTAC | 2053 | GTAGCAACATATGCTACAC |
| siRNA 312 | 312 | TGTAGCATATGTTGCTACC | 2054 | GGTAGCAACATATGCTACA |
| siRNA 313 | 313 | GTAGCATATGTTGCTACCA | 2055 | TGGTAGCAACATATGCTAC |
| siRNA 314 | 314 | TAGCATATGTTGCTACCAG | 2056 | CTGGTAGCAACATATGCTA |
| siRNA 315 | 315 | AGCATATGTTGCTACCAGA | 2057 | TCTGGTAGCAACATATGCT |
| siRNA 316 | 316 | GCATATGTTGCTACCAGAG | 2058 | CTCTGGTAGCAACATATGC |
| siRNA 317 | 317 | CATATGTTGCTACCAGAGA | 2059 | TCTCTGGTAGCAACATATG |
| siRNA 318 | 318 | ATATGTTGCTACCAGAGAC | 2060 | GTCTCTGGTAGCAACATAT |
| siRNA 319 | 319 | TATGTTGCTACCAGAGACA | 2061 | TGTCTCTGGTAGCAACATA |
| siRNA 320 | 320 | ATGTTGCTACCAGAGACAA | 2062 | TTGTCTCTGGTAGCAACAT |
| siRNA 321 | 321 | TGTTGCTACCAGAGACAAC | 2063 | GTTGTCTCTGGTAGCAACA |
| siRNA 322 | 322 | GTTGCTACCAGAGACAACT | 2064 | AGTTGTCTCTGGTAGCAAC |
| siRNA 323 | 323 | TTGCTACCAGAGACAACTG | 2065 | CAGTTGTCTCTGGTAGCAA |
| siRNA 324 | 324 | TGCTACCAGAGACAACTGC | 2066 | GCAGTTGTCTCTGGTAGCA |
| siRNA 325 | 325 | GCTACCAGAGACAACTGCT | 2067 | AGCAGTTGTCTCTGGTAGC |
| siRNA 326 | 326 | CTACCAGAGACAACTGCTG | 2068 | CAGCAGTTGTCTCTGGTAG |
| siRNA 327 | 327 | TACCAGAGACAACTGCTGC | 2069 | GCAGCAGTTGTCTCTGGTA |
| siRNA 328 | 328 | ACCAGAGACAACTGCTGCA | 2070 | TGCAGCAGTTGTCTCTGGT |
| siRNA 329 | 329 | CCAGAGACAACTGCTGCAT | 2071 | ATGCAGCAGTTGTCTCTGG |
| siRNA 330 | 330 | CAGAGACAACTGCTGCATC | 2072 | GATGCAGCAGTTGTCTCTG |
| siRNA 331 | 331 | AGAGACAACTGCTGCATCT | 2073 | AGATGCAGCAGTTGTCTCT |
| siRNA 332 | 332 | GAGACAACTGCTGCATCTT | 2074 | AAGATGCAGCAGTTGTCTC |
| siRNA 333 | 333 | AGACAACTGCTGCATCTTA | 2075 | TAAGATGCAGCAGTTGTCT |
| siRNA 334 | 334 | GACAACTGCTGCATCTTAG | 2076 | CTAAGATGCAGCAGTTGTC |
| siRNA 335 | 335 | ACAACTGCTGCATCTTAGA | 2077 | TCTAAGATGCAGCAGTTGT |
| siRNA 336 | 336 | CAACTGCTGCATCTTAGAT | 2078 | ATCTAAGATGCAGCAGTTG |
| siRNA 337 | 337 | AACTGCTGCATCTTAGATG | 2079 | CATCTAAGATGCAGCAGTT |
| siRNA 338 | 338 | ACTGCTGCATCTTAGATGA | 2080 | TCATCTAAGATGCAGCAGT |
| siRNA 339 | 339 | CTGCTGCATCTTAGATGAA | 2081 | TTCATCTAAGATGCAGCAG |
| siRNA 340 | 340 | TGCTGCATCTTAGATGAAA | 2082 | TTTCATCTAAGATGCAGCA |
| siRNA 341 | 341 | GCTGCATCTTAGATGAAAG | 2083 | CTTTCATCTAAGATGCAGC |
| siRNA 342 | 342 | CTGCATCTTAGATGAAAGA | 2084 | TCTTTCATCTAAGATGCAG |
| siRNA 343 | 343 | TGCATCTTAGATGAAAGAT | 2085 | ATCTTTCATCTAAGATGCA |
| siRNA 344 | 344 | GCATCTTAGATGAAAGATT | 2086 | AATCTTTCATCTAAGATGC |
| siRNA 345 | 345 | CATCTTAGATGAAAGATTC | 2087 | GAATCTTTCATCTAAGATG |
| siRNA 346 | 346 | ATCTTAGATGAAAGATTCG | 2088 | CGAATCTTTCATCTAAGAT |
| siRNA 347 | 347 | TCTTAGATGAAAGATTCGG | 2089 | CCGAATCTTTCATCTAAGA |
| siRNA 348 | 348 | CTTAGATGAAAGATTCGGT | 2090 | ACCGAATCTTTCATCTAAG |
| siRNA 349 | 349 | TTAGATGAAAGATTCGGTA | 2091 | TACCGAATCTTTCATCTAA |
| siRNA 350 | 350 | TAGATGAAAGATTCGGTAG | 2092 | CTACCGAATCTTTCATCTA |
| siRNA 351 | 351 | AGATGAAAGATTCGGTAGT | 2093 | ACTACCGAATCTTTCATCT |
| siRNA 352 | 352 | GATGAAAGATTCGGTAGTT | 2094 | AACTACCGAATCTTTCATC |
| siRNA 353 | 353 | ATGAAAGATTCGGTAGTTA | 2095 | TAACTACCGAATCTTTCAT |
| siRNA 354 | 354 | TGAAAGATTCGGTAGTTAT | 2096 | ATAACTACCGAATCTTTCA |
| siRNA 355 | 355 | GAAAGATTCGGTAGTTATT | 2097 | AATAACTACCGAATCTTTC |
| siRNA 356 | 356 | AAAGATTCGGTAGTTATTG | 2098 | CAATAACTACCGAATCTTT |
| siRNA 357 | 357 | AAGATTCGGTAGTTATTGT | 2099 | ACAATAACTACCGAATCTT |
| siRNA 358 | 358 | AGATTCGGTAGTTATTGTC | 2100 | GACAATAACTACCGAATCT |
| siRNA 359 | 359 | GATTCGGTAGTTATTGTCC | 2101 | GGACAATAACTACCGAATC |
| siRNA 360 | 360 | ATTCGGTAGTTATTGTCCA | 2102 | TGGACAATAACTACCGAAT |
| siRNA 361 | 361 | TTCGGTAGTTATTGTCCAA | 2103 | TTGGACAATAACTACCGAA |
| siRNA 362 | 362 | TCGGTAGTTATTGTCCAAC | 2104 | GTTGGACAATAACTACCGA |
| siRNA 363 | 363 | CGGTAGTTATTGTCCAACT | 2105 | AGTTGGACAATAACTACCG |
| siRNA 364 | 364 | GGTAGTTATTGTCCAACTA | 2106 | TAGTTGGACAATAACTACC |
| siRNA 365 | 365 | GTAGTTATTGTCCAACTAC | 2107 | GTAGTTGGACAATAACTAC |
| siRNA 366 | 366 | TAGTTATTGTCCAACTACC | 2108 | GGTAGTTGGACAATAACTA |
| siRNA 367 | 367 | AGTTATTGTCCAACTACCT | 2109 | AGGTAGTTGGACAATAACT |
| siRNA 368 | 368 | GTTATTGTCCAACTACCTG | 2110 | CAGGTAGTTGGACAATAAC |
| siRNA 369 | 369 | TTATTGTCCAACTACCTGT | 2111 | ACAGGTAGTTGGACAATAA |
| siRNA 370 | 370 | TATTGTCCAACTACCTGTG | 2112 | CACAGGTAGTTGGACAATA |
| siRNA 371 | 371 | ATTGTCCAACTACCTGTGG | 2113 | CCACAGGTAGTTGGACAAT |
| siRNA 372 | 372 | TTGTCCAACTACCTGTGGC | 2114 | GCCACAGGTAGTTGGACAA |
| siRNA 373 | 373 | TGTCCAACTACCTGTGGCA | 2115 | TGCCACAGGTAGTTGGACA |
| siRNA 374 | 374 | GTCCAACTACCTGTGGCAT | 2116 | ATGCCACAGGTAGTTGGAC |
| siRNA 375 | 375 | TCCAACTACCTGTGGCATT | 2117 | AATGCCACAGGTAGTTGGA |
| siRNA 376 | 376 | CCAACTACCTGTGGCATTG | 2118 | CAATGCCACAGGTAGTTGG |
| siRNA 377 | 377 | CAACTACCTGTGGCATTGC | 2119 | GCAATGCCACAGGTAGTTG |
| siRNA 378 | 378 | AACTACCTGTGGCATTGCA | 2120 | TGCAATGCCACAGGTAGTT |
| siRNA 379 | 379 | ACTACCTGTGGCATTGCAG | 2121 | CTGCAATGCCACAGGTAGT |
| siRNA 380 | 380 | CTACCTGTGGCATTGCAGA | 2122 | TCTGCAATGCCACAGGTAG |
| siRNA 381 | 381 | TACCTGTGGCATTGCAGAT | 2123 | ATCTGCAATGCCACAGGTA |
| siRNA 382 | 382 | ACCTGTGGCATTGCAGATT | 2124 | AATCTGCAATGCCACAGGT |
| siRNA 383 | 383 | CCTGTGGCATTGCAGATTT | 2125 | AAATCTGCAATGCCACAGG |
| siRNA 384 | 384 | CTGTGGCATTGCAGATTTC | 2126 | GAAATCTGCAATGCCACAG |
| siRNA 385 | 385 | TGTGGCATTGCAGATTTCC | 2127 | GGAAATCTGCAATGCCACA |
| siRNA 386 | 386 | GTGGCATTGCAGATTTCCT | 2128 | AGGAAATCTGCAATGCCAC |
| siRNA 387 | 387 | TGGCATTGCAGATTTCCTG | 2129 | CAGGAAATCTGCAATGCCA |
| siRNA 388 | 388 | GGCATTGCAGATTTCCTGT | 2130 | ACAGGAAATCTGCAATGCC |
| siRNA 389 | 389 | GCATTGCAGATTTCCTGTC | 2131 | GACAGGAAATCTGCAATGC |
| siRNA 390 | 390 | CATTGCAGATTTCCTGTCT | 2132 | AGACAGGAAATCTGCAATG |
| siRNA 391 | 391 | ATTGCAGATTTCCTGTCTA | 2133 | TAGACAGGAAATCTGCAAT |
| siRNA 392 | 392 | TTGCAGATTTCCTGTCTAC | 2134 | GTAGACAGGAAATCTGCAA |
| siRNA 393 | 393 | TGCAGATTTCCTGTCTACT | 2135 | AGTAGACAGGAAATCTGCA |
| siRNA 394 | 394 | GCAGATTTCCTGTCTACTT | 2136 | AAGTAGACAGGAAATCTGC |
| siRNA 395 | 395 | CAGATTTCCTGTCTACTTA | 2137 | TAAGTAGACAGGAAATCTG |
| siRNA 396 | 396 | AGATTTCCTGTCTACTTAT | 2138 | ATAAGTAGACAGGAAATCT |
| siRNA 397 | 397 | GATTTCCTGTCTACTTATC | 2139 | GATAAGTAGACAGGAAATC |
| siRNA 398 | 398 | ATTTCCTGTCTACTTATCA | 2140 | TGATAAGTAGACAGGAAAT |
| siRNA 399 | 399 | TTTCCTGTCTACTTATCAA | 2141 | TTGATAAGTAGACAGGAAA |
| siRNA 400 | 400 | TTCCTGTCTACTTATCAAA | 2142 | TTTGATAAGTAGACAGGAA |
| siRNA 401 | 401 | TCCTGTCTACTTATCAAAC | 2143 | GTTTGATAAGTAGACAGGA |
| siRNA 402 | 402 | CCTGTCTACTTATCAAACC | 2144 | GGTTTGATAAGTAGACAGG |
| siRNA 403 | 403 | CTGTCTACTTATCAAACCA | 2145 | TGGTTTGATAAGTAGACAG |
| siRNA 404 | 404 | TGTCTACTTATCAAACCAA | 2146 | TTGGTTTGATAAGTAGACA |
| siRNA 405 | 405 | GTCTACTTATCAAACCAAA | 2147 | TTTGGTTTGATAAGTAGAC |
| siRNA 406 | 406 | TCTACTTATCAAACCAAAG | 2148 | CTTTGGTTTGATAAGTAGA |
| siRNA 407 | 407 | CTACTTATCAAACCAAAGT | 2149 | ACTTTGGTTTGATAAGTAG |
| siRNA 408 | 408 | TACTTATCAAACCAAAGTA | 2150 | TACTTTGGTTTGATAAGTA |
| siRNA 409 | 409 | ACTTATCAAACCAAAGTAG | 2151 | CTACTTTGGTTTGATAAGT |
| siRNA 410 | 410 | CTTATCAAACCAAAGTAGA | 2152 | TCTACTTTGGTTTGATAAG |
| siRNA 411 | 411 | TTATCAAACCAAAGTAGAC | 2153 | GTCTACTTTGGTTTGATAA |
| siRNA 412 | 412 | TATCAAACCAAAGTAGACA | 2154 | TGTCTACTTTGGTTTGATA |
| siRNA 413 | 413 | ATCAAACCAAAGTAGACAA | 2155 | TTGTCTACTTTGGTTTGAT |
| siRNA 414 | 414 | TCAAACCAAAGTAGACAAG | 2156 | CTTGTCTACTTTGGTTTGA |
| siRNA 415 | 415 | CAAACCAAAGTAGACAAGG | 2157 | CCTTGTCTACTTTGGTTTG |
| siRNA 416 | 416 | AAACCAAAGTAGACAAGGA | 2158 | TCCTTGTCTACTTTGGTTT |
| siRNA 417 | 417 | AACCAAAGTAGACAAGGAT | 2159 | ATCCTTGTCTACTTTGGTT |
| siRNA 418 | 418 | ACCAAAGTAGACAAGGATC | 2160 | GATCCTTGTCTACTTTGGT |
| siRNA 419 | 419 | CCAAAGTAGACAAGGATCT | 2161 | AGATCCTTGTCTACTTTGG |
| siRNA 420 | 420 | CAAAGTAGACAAGGATCTA | 2162 | TAGATCCTTGTCTACTTTG |
| siRNA 421 | 421 | AAAGTAGACAAGGATCTAC | 2163 | GTAGATCCTTGTCTACTTT |
| siRNA 422 | 422 | AAGTAGACAAGGATCTACA | 2164 | TGTAGATCCTTGTCTACTT |
| siRNA 423 | 423 | AGTAGACAAGGATCTACAG | 2165 | CTGTAGATCCTTGTCTACT |
| siRNA 424 | 424 | GTAGACAAGGATCTACAGT | 2166 | ACTGTAGATCCTTGTCTAC |
| siRNA 425 | 425 | TAGACAAGGATCTACAGTC | 2167 | GACTGTAGATCCTTGTCTA |
| siRNA 426 | 426 | AGACAAGGATCTACAGTCT | 2168 | AGACTGTAGATCCTTGTCT |
| siRNA 427 | 427 | GACAAGGATCTACAGTCTT | 2169 | AAGACTGTAGATCCTTGTC |
| siRNA 428 | 428 | ACAAGGATCTACAGTCTTT | 2170 | AAAGACTGTAGATCCTTGT |
| siRNA 429 | 429 | CAAGGATCTACAGTCTTTG | 2171 | CAAAGACTGTAGATCCTTG |
| siRNA 430 | 430 | AAGGATCTACAGTCTTTGG | 2172 | CCAAAGACTGTAGATCCTT |
| siRNA 431 | 431 | AGGATCTACAGTCTTTGGA | 2173 | TCCAAAGACTGTAGATCCT |
| siRNA 432 | 432 | GGATCTACAGTCTTTGGAA | 2174 | TTCCAAAGACTGTAGATCC |
| siRNA 433 | 433 | GATCTACAGTCTTTGGAAG | 2175 | CTTCCAAAGACTGTAGATC |
| siRNA 434 | 434 | ATCTACAGTCTTTGGAAGA | 2176 | TCTTCCAAAGACTGTAGAT |
| siRNA 435 | 435 | TCTACAGTCTTTGGAAGAC | 2177 | GTCTTCCAAAGACTGTAGA |
| siRNA 436 | 436 | CTACAGTCTTTGGAAGACA | 2178 | TGTCTTCCAAAGACTGTAG |
| siRNA 437 | 437 | TACAGTCTTTGGAAGACAT | 2179 | ATGTCTTCCAAAGACTGTA |
| siRNA 438 | 438 | ACAGTCTTTGGAAGACATC | 2180 | GATGTCTTCCAAAGACTGT |
| siRNA 439 | 439 | CAGTCTTTGGAAGACATCT | 2181 | AGATGTCTTCCAAAGACTG |
| siRNA 440 | 440 | AGTCTTTGGAAGACATCTT | 2182 | AAGATGTCTTCCAAAGACT |
| siRNA 441 | 441 | GTCTTTGGAAGACATCTTA | 2183 | TAAGATGTCTTCCAAAGAC |
| siRNA 442 | 442 | TCTTTGGAAGACATCTTAC | 2184 | GTAAGATGTCTTCCAAAGA |
| siRNA 443 | 443 | CTTTGGAAGACATCTTACA | 2185 | TGTAAGATGTCTTCCAAAG |
| siRNA 444 | 444 | TTTGGAAGACATCTTACAT | 2186 | ATGTAAGATGTCTTCCAAA |
| siRNA 445 | 445 | TTGGAAGACATCTTACATC | 2187 | GATGTAAGATGTCTTCCAA |
| siRNA 446 | 446 | TGGAAGACATCTTACATCA | 2188 | TGATGTAAGATGTCTTCCA |
| siRNA 447 | 447 | GGAAGACATCTTACATCAA | 2189 | TTGATGTAAGATGTCTTCC |
| siRNA 448 | 448 | GAAGACATCTTACATCAAG | 2190 | CTTGATGTAAGATGTCTTC |
| siRNA 449 | 449 | AAGACATCTTACATCAAGT | 2191 | ACTTGATGTAAGATGTCTT |
| siRNA 450 | 450 | AGACATCTTACATCAAGTT | 2192 | AACTTGATGTAAGATGTCT |
| siRNA 451 | 451 | GACATCTTACATCAAGTTG | 2193 | CAACTTGATGTAAGATGTC |
| siRNA 452 | 452 | ACATCTTACATCAAGTTGA | 2194 | TCAACTTGATGTAAGATGT |
| siRNA 453 | 453 | CATCTTACATCAAGTTGAA | 2195 | TTCAACTTGATGTAAGATG |
| siRNA 454 | 454 | ATCTTACATCAAGTTGAAA | 2196 | TTTCAACTTGATGTAAGAT |
| siRNA 455 | 455 | TCTTACATCAAGTTGAAAA | 2197 | TTTTCAACTTGATGTAAGA |
| siRNA 456 | 456 | CTTACATCAAGTTGAAAAC | 2198 | GTTTTCAACTTGATGTAAG |
| siRNA 457 | 457 | TTACATCAAGTTGAAAACA | 2199 | TGTTTTCAACTTGATGTAA |
| siRNA 458 | 458 | TACATCAAGTTGAAAACAA | 2200 | TTGTTTTCAACTTGATGTA |
| siRNA 459 | 459 | ACATCAAGTTGAAAACAAA | 2201 | TTTGTTTTCAACTTGATGT |
| siRNA 460 | 460 | CATCAAGTTGAAAACAAAA | 2202 | TTTTGTTTTCAACTTGATG |
| siRNA 461 | 461 | ATCAAGTTGAAAACAAAAC | 2203 | GTTTTGTTTTCAACTTGAT |
| siRNA 462 | 462 | TCAAGTTGAAAACAAAACA | 2204 | TGTTTTGTTTTCAACTTGA |
| siRNA 463 | 463 | CAAGTTGAAAACAAAACAT | 2205 | ATGTTTTGTTTTCAACTTG |
| siRNA 464 | 464 | AAGTTGAAAACAAAACATC | 2206 | GATGTTTTGTTTTCAACTT |
| siRNA 465 | 465 | AGTTGAAAACAAAACATCA | 2207 | TGATGTTTTGTTTTCAACT |
| siRNA 466 | 466 | GTTGAAAACAAAACATCAG | 2208 | CTGATGTTTTGTTTTCAAC |
| siRNA 467 | 467 | TTGAAAACAAAACATCAGA | 2209 | TCTGATGTTTTGTTTTCAA |
| siRNA 468 | 468 | TGAAAACAAAACATCAGAA | 2210 | TTCTGATGTTTTGTTTTCA |
| siRNA 469 | 469 | GAAAACAAAACATCAGAAG | 2211 | CTTCTGATGTTTTGTTTTC |
| siRNA 470 | 470 | AAAACAAAACATCAGAAGT | 2212 | ACTTCTGATGTTTTGTTTT |
| siRNA 471 | 471 | AAACAAAACATCAGAAGTC | 2213 | GACTTCTGATGTTTTGTTT |
| siRNA 472 | 472 | AACAAAACATCAGAAGTCA | 2214 | TGACTTCTGATGTTTTGTT |
| siRNA 473 | 473 | ACAAAACATCAGAAGTCAA | 2215 | TTGACTTCTGATGTTTTGT |
| siRNA 474 | 474 | CAAAACATCAGAAGTCAAA | 2216 | TTTGACTTCTGATGTTTTG |
| siRNA 475 | 475 | AAAACATCAGAAGTCAAAC | 2217 | GTTTGACTTCTGATGTTTT |
| siRNA 476 | 476 | AAACATCAGAAGTCAAACA | 2218 | TGTTTGACTTCTGATGTTT |
| siRNA 477 | 477 | AACATCAGAAGTCAAACAG | 2219 | CTGTTTGACTTCTGATGTT |
| siRNA 478 | 478 | ACATCAGAAGTCAAACAGC | 2220 | GCTGTTTGACTTCTGATGT |
| siRNA 479 | 479 | CATCAGAAGTCAAACAGCT | 2221 | AGCTGTTTGACTTCTGATG |
| siRNA 480 | 480 | ATCAGAAGTCAAACAGCTG | 2222 | CAGCTGTTTGACTTCTGAT |
| siRNA 481 | 481 | TCAGAAGTCAAACAGCTGA | 2223 | TCAGCTGTTTGACTTCTGA |
| siRNA 482 | 482 | CAGAAGTCAAACAGCTGAT | 2224 | ATCAGCTGTTTGACTTCTG |
| siRNA 483 | 483 | AGAAGTCAAACAGCTGATA | 2225 | TATCAGCTGTTTGACTTCT |
| siRNA 484 | 484 | GAAGTCAAACAGCTGATAA | 2226 | TTATCAGCTGTTTGACTTC |
| siRNA 485 | 485 | AAGTCAAACAGCTGATAAA | 2227 | TTTATCAGCTGTTTGACTT |
| siRNA 486 | 486 | AGTCAAACAGCTGATAAAA | 2228 | TTTTATCAGCTGTTTGACT |
| siRNA 487 | 487 | GTCAAACAGCTGATAAAAG | 2229 | CTTTTATCAGCTGTTTGAC |
| siRNA 488 | 488 | TCAAACAGCTGATAAAAGC | 2230 | GCTTTTATCAGCTGTTTGA |
| siRNA 489 | 489 | CAAACAGCTGATAAAAGCA | 2231 | TGCTTTTATCAGCTGTTTG |
| siRNA 490 | 490 | AAACAGCTGATAAAAGCAA | 2232 | TTGCTTTTATCAGCTGTTT |
| siRNA 491 | 491 | AACAGCTGATAAAAGCAAT | 2233 | ATTGCTTTTATCAGCTGTT |
| siRNA 492 | 492 | ACAGCTGATAAAAGCAATC | 2234 | GATTGCTTTTATCAGCTGT |
| siRNA 493 | 493 | CAGCTGATAAAAGCAATCC | 2235 | GGATTGCTTTTATCAGCTG |
| siRNA 494 | 494 | AGCTGATAAAAGCAATCCA | 2236 | TGGATTGCTTTTATCAGCT |
| siRNA 495 | 495 | GCTGATAAAAGCAATCCAA | 2237 | TTGGATTGCTTTTATCAGC |
| siRNA 496 | 496 | CTGATAAAAGCAATCCAAC | 2238 | GTTGGATTGCTTTTATCAG |
| siRNA 497 | 497 | TGATAAAAGCAATCCAACT | 2239 | AGTTGGATTGCTTTTATCA |
| siRNA 498 | 498 | GATAAAAGCAATCCAACTC | 2240 | GAGTTGGATTGCTTTTATC |
| siRNA 499 | 499 | ATAAAAGCAATCCAACTCA | 2241 | TGAGTTGGATTGCTTTTAT |
| siRNA 500 | 500 | TAAAAGCAATCCAACTCAC | 2242 | GTGAGTTGGATTGCTTTTA |
| siRNA 501 | 501 | AAAAGCAATCCAACTCACT | 2243 | AGTGAGTTGGATTGCTTTT |
| siRNA 502 | 502 | AAAGCAATCCAACTCACTT | 2244 | AAGTGAGTTGGATTGCTTT |
| siRNA 503 | 503 | AAGCAATCCAACTCACTTA | 2245 | TAAGTGAGTTGGATTGCTT |
| siRNA 504 | 504 | AGCAATCCAACTCACTTAT | 2246 | ATAAGTGAGTTGGATTGCT |
| siRNA 505 | 505 | GCAATCCAACTCACTTATA | 2247 | TATAAGTGAGTTGGATTGC |
| siRNA 506 | 506 | CAATCCAACTCACTTATAA | 2248 | TTATAAGTGAGTTGGATTG |
| siRNA 507 | 507 | AATCCAACTCACTTATAAT | 2249 | ATTATAAGTGAGTTGGATT |
| siRNA 508 | 508 | ATCCAACTCACTTATAATC | 2250 | GATTATAAGTGAGTTGGAT |
| siRNA 509 | 509 | TCCAACTCACTTATAATCC | 2251 | GGATTATAAGTGAGTTGGA |
| siRNA 510 | 510 | CCAACTCACTTATAATCCT | 2252 | AGGATTATAAGTGAGTTGG |
| siRNA 511 | 511 | CAACTCACTTATAATCCTG | 2253 | CAGGATTATAAGTGAGTTG |
| siRNA 512 | 512 | AACTCACTTATAATCCTGA | 2254 | TCAGGATTATAAGTGAGTT |
| siRNA 513 | 513 | ACTCACTTATAATCCTGAT | 2255 | ATCAGGATTATAAGTGAGT |
| siRNA 514 | 514 | CTCACTTATAATCCTGATG | 2256 | CATCAGGATTATAAGTGAG |
| siRNA 515 | 515 | TCACTTATAATCCTGATGA | 2257 | TCATCAGGATTATAAGTGA |
| siRNA 516 | 516 | CACTTATAATCCTGATGAA | 2258 | TTCATCAGGATTATAAGTG |
| siRNA 517 | 517 | ACTTATAATCCTGATGAAT | 2259 | ATTCATCAGGATTATAAGT |
| siRNA 518 | 518 | CTTATAATCCTGATGAATC | 2260 | GATTCATCAGGATTATAAG |
| siRNA 519 | 519 | TTATAATCCTGATGAATCA | 2261 | TGATTCATCAGGATTATAA |
| siRNA 520 | 520 | TATAATCCTGATGAATCAT | 2262 | ATGATTCATCAGGATTATA |
| siRNA 521 | 521 | ATAATCCTGATGAATCATC | 2263 | GATGATTCATCAGGATTAT |
| siRNA 522 | 522 | TAATCCTGATGAATCATCA | 2264 | TGATGATTCATCAGGATTA |
| siRNA 523 | 523 | AATCCTGATGAATCATCAA | 2265 | TTGATGATTCATCAGGATT |
| siRNA 524 | 524 | ATCCTGATGAATCATCAAA | 2266 | TTTGATGATTCATCAGGAT |
| siRNA 525 | 525 | TCCTGATGAATCATCAAAA | 2267 | TTTTGATGATTCATCAGGA |
| siRNA 526 | 526 | CCTGATGAATCATCAAAAC | 2268 | GTTTTGATGATTCATCAGG |
| siRNA 527 | 527 | CTGATGAATCATCAAAACC | 2269 | GGTTTTGATGATTCATCAG |
| siRNA 528 | 528 | TGATGAATCATCAAAACCA | 2270 | TGGTTTTGATGATTCATCA |
| siRNA 529 | 529 | GATGAATCATCAAAACCAA | 2271 | TTGGTTTTGATGATTCATC |
| siRNA 530 | 530 | ATGAATCATCAAAACCAAA | 2272 | TTTGGTTTTGATGATTCAT |
| siRNA 531 | 531 | TGAATCATCAAAACCAAAT | 2273 | ATTTGGTTTTGATGATTCA |
| siRNA 532 | 532 | GAATCATCAAAACCAAATA | 2274 | TATTTGGTTTTGATGATTC |
| siRNA 533 | 533 | AATCATCAAAACCAAATAT | 2275 | ATATTTGGTTTTGATGATT |
| siRNA 534 | 534 | ATCATCAAAACCAAATATG | 2276 | CATATTTGGTTTTGATGAT |
| siRNA 535 | 535 | TCATCAAAACCAAATATGA | 2277 | TCATATTTGGTTTTGATGA |
| siRNA 536 | 536 | CATCAAAACCAAATATGAT | 2278 | ATCATATTTGGTTTTGATG |
| siRNA 537 | 537 | ATCAAAACCAAATATGATA | 2279 | TATCATATTTGGTTTTGAT |
| siRNA 538 | 538 | TCAAAACCAAATATGATAG | 2280 | CTATCATATTTGGTTTTGA |
| siRNA 539 | 539 | CAAAACCAAATATGATAGA | 2281 | TCTATCATATTTGGTTTTG |
| siRNA 540 | 540 | AAAACCAAATATGATAGAC | 2282 | GTCTATCATATTTGGTTTT |
| siRNA 541 | 541 | AAACCAAATATGATAGACG | 2283 | CGTCTATCATATTTGGTTT |
| siRNA 542 | 542 | AACCAAATATGATAGACGC | 2284 | GCGTCTATCATATTTGGTT |
| siRNA 543 | 543 | ACCAAATATGATAGACGCT | 2285 | AGCGTCTATCATATTTGGT |
| siRNA 544 | 544 | CCAAATATGATAGACGCTG | 2286 | CAGCGTCTATCATATTTGG |
| siRNA 545 | 545 | CAAATATGATAGACGCTGC | 2287 | GCAGCGTCTATCATATTTG |
| siRNA 546 | 546 | AAATATGATAGACGCTGCT | 2288 | AGCAGCGTCTATCATATTT |
| siRNA 547 | 547 | AATATGATAGACGCTGCTA | 2289 | TAGCAGCGTCTATCATATT |
| siRNA 548 | 548 | ATATGATAGACGCTGCTAC | 2290 | GTAGCAGCGTCTATCATAT |
| siRNA 549 | 549 | TATGATAGACGCTGCTACT | 2291 | AGTAGCAGCGTCTATCATA |
| siRNA 550 | 550 | ATGATAGACGCTGCTACTT | 2292 | AAGTAGCAGCGTCTATCAT |
| siRNA 551 | 551 | TGATAGACGCTGCTACTTT | 2293 | AAAGTAGCAGCGTCTATCA |
| siRNA 552 | 552 | GATAGACGCTGCTACTTTG | 2294 | CAAAGTAGCAGCGTCTATC |
| siRNA 553 | 553 | ATAGACGCTGCTACTTTGA | 2295 | TCAAAGTAGCAGCGTCTAT |
| siRNA 554 | 554 | TAGACGCTGCTACTTTGAA | 2296 | TTCAAAGTAGCAGCGTCTA |
| siRNA 555 | 555 | AGACGCTGCTACTTTGAAG | 2297 | CTTCAAAGTAGCAGCGTCT |
| siRNA 556 | 556 | GACGCTGCTACTTTGAAGT | 2298 | ACTTCAAAGTAGCAGCGTC |
| siRNA 557 | 557 | ACGCTGCTACTTTGAAGTC | 2299 | GACTTCAAAGTAGCAGCGT |
| siRNA 558 | 558 | CGCTGCTACTTTGAAGTCC | 2300 | GGACTTCAAAGTAGCAGCG |
| siRNA 559 | 559 | GCTGCTACTTTGAAGTCCA | 2301 | TGGACTTCAAAGTAGCAGC |
| siRNA 560 | 560 | CTGCTACTTTGAAGTCCAG | 2302 | CTGGACTTCAAAGTAGCAG |
| siRNA 561 | 561 | TGCTACTTTGAAGTCCAGG | 2303 | CCTGGACTTCAAAGTAGCA |
| siRNA 562 | 562 | GCTACTTTGAAGTCCAGGA | 2304 | TCCTGGACTTCAAAGTAGC |
| siRNA 563 | 563 | CTACTTTGAAGTCCAGGAA | 2305 | TTCCTGGACTTCAAAGTAG |
| siRNA 564 | 564 | TACTTTGAAGTCCAGGAAA | 2306 | TTTCCTGGACTTCAAAGTA |
| siRNA 565 | 565 | ACTTTGAAGTCCAGGAAAA | 2307 | TTTTCCTGGACTTCAAAGT |
| siRNA 566 | 566 | CTTTGAAGTCCAGGAAAAT | 2308 | ATTTTCCTGGACTTCAAAG |
| siRNA 567 | 567 | TTTGAAGTCCAGGAAAATG | 2309 | CATTTTCCTGGACTTCAAA |
| siRNA 568 | 568 | TTGAAGTCCAGGAAAATGT | 2310 | ACATTTTCCTGGACTTCAA |
| siRNA 569 | 569 | TGAAGTCCAGGAAAATGTT | 2311 | AACATTTTCCTGGACTTCA |
| siRNA 570 | 570 | GAAGTCCAGGAAAATGTTA | 2312 | TAACATTTTCCTGGACTTC |
| siRNA 571 | 571 | AAGTCCAGGAAAATGTTAG | 2313 | CTAACATTTTCCTGGACTT |
| siRNA 572 | 572 | AGTCCAGGAAAATGTTAGA | 2314 | TCTAACATTTTCCTGGACT |
| siRNA 573 | 573 | GTCCAGGAAAATGTTAGAA | 2315 | TTCTAACATTTTCCTGGAC |
| siRNA 574 | 574 | TCCAGGAAAATGTTAGAAG | 2316 | CTTCTAACATTTTCCTGGA |
| siRNA 575 | 575 | CCAGGAAAATGTTAGAAGA | 2317 | TCTTCTAACATTTTCCTGG |
| siRNA 576 | 576 | CAGGAAAATGTTAGAAGAA | 2318 | TTCTTCTAACATTTTCCTG |
| siRNA 577 | 577 | AGGAAAATGTTAGAAGAAA | 2319 | TTTCTTCTAACATTTTCCT |
| siRNA 578 | 578 | GGAAAATGTTAGAAGAAAT | 2320 | ATTTCTTCTAACATTTTCC |
| siRNA 579 | 579 | GAAAATGTTAGAAGAAATT | 2321 | AATTTCTTCTAACATTTTC |
| siRNA 580 | 580 | AAAATGTTAGAAGAAATTA | 2322 | TAATTTCTTCTAACATTTT |
| siRNA 581 | 581 | AAATGTTAGAAGAAATTAT | 2323 | ATAATTTCTTCTAACATTT |
| siRNA 582 | 582 | AATGTTAGAAGAAATTATG | 2324 | CATAATTTCTTCTAACATT |
| siRNA 583 | 583 | ATGTTAGAAGAAATTATGA | 2325 | TCATAATTTCTTCTAACAT |
| siRNA 584 | 584 | TGTTAGAAGAAATTATGAA | 2326 | TTCATAATTTCTTCTAACA |
| siRNA 585 | 585 | GTTAGAAGAAATTATGAAA | 2327 | TTTCATAATTTCTTCTAAC |
| siRNA 586 | 586 | TTAGAAGAAATTATGAAAT | 2328 | ATTTCATAATTTCTTCTAA |
| siRNA 587 | 587 | TAGAAGAAATTATGAAATA | 2329 | TATTTCATAATTTCTTCTA |
| siRNA 588 | 588 | AGAAGAAATTATGAAATAT | 2330 | ATATTTCATAATTTCTTCT |
| siRNA 589 | 589 | GAAGAAATTATGAAATATG | 2331 | CATATTTCATAATTTCTTC |
| siRNA 590 | 590 | AAGAAATTATGAAATATGA | 2332 | TCATATTTCATAATTTCTT |
| siRNA 591 | 591 | AGAAATTATGAAATATGAA | 2333 | TTCATATTTCATAATTTCT |
| siRNA 592 | 592 | GAAATTATGAAATATGAAG | 2334 | CTTCATATTTCATAATTTC |
| siRNA 593 | 593 | AAATTATGAAATATGAAGC | 2335 | GCTTCATATTTCATAATTT |
| siRNA 594 | 594 | AATTATGAAATATGAAGCA | 2336 | TGCTTCATATTTCATAATT |
| siRNA 595 | 595 | ATTATGAAATATGAAGCAT | 2337 | ATGCTTCATATTTCATAAT |
| siRNA 596 | 596 | TTATGAAATATGAAGCATC | 2338 | GATGCTTCATATTICATAA |
| siRNA 597 | 597 | TATGAAATATGAAGCATCG | 2339 | CGATGCTTCATATTTCATA |
| siRNA 598 | 598 | ATGAAATATGAAGCATCGA | 2340 | TCGATGCTTCATATTTCAT |
| siRNA 599 | 599 | TGAAATATGAAGCATCGAT | 2341 | ATCGATGCTTCATATTTCA |
| siRNA 600 | 600 | GAAATATGAAGCATCGATT | 2342 | AATCGATGCTTCATATTTC |
| siRNA 601 | 601 | AAATATGAAGCATCGATTT | 2343 | AAATCGATGCTTCATATTT |
| siRNA 602 | 602 | AATATGAAGCATCGATTTT | 2344 | AAAATCGATGCTTCATATT |
| siRNA 603 | 603 | ATATGAAGCATCGATTTTA | 2345 | TAAAATCGATGCTTCATAT |
| siRNA 604 | 604 | TATGAAGCATCGATTTTAA | 2346 | TTAAAATCGATGCTTCATA |
| siRNA 605 | 605 | ATGAAGCATCGATTTTAAC | 2347 | GTTAAAATCGATGCTTCAT |
| siRNA 606 | 606 | TGAAGCATCGATTTTAACA | 2348 | TGTTAAAATCGATGCTTCA |
| siRNA 607 | 607 | GAAGCATCGATTTTAACAC | 2349 | GTGTTAAAATCGATGCTTC |
| siRNA 608 | 608 | AAGCATCGATTTTAACACA | 2350 | TGTGTTAAAATCGATGCTT |
| siRNA 609 | 609 | AGCATCGATTTTAACACAT | 2351 | ATGTGTTAAAATCGATGCT |
| siRNA 610 | 610 | GCATCGATTTTAACACATG | 2352 | CATGTGTTAAAATCGATGC |
| siRNA 611 | 611 | CATCGATTTTAACACATGA | 2353 | TCATGTGTTAAAATCGATG |
| siRNA 612 | 612 | ATCGATTTTAACACATGAC | 2354 | GTCATGTGTTAAAATCGAT |
| siRNA 613 | 613 | TCGATTTTAACACATGACT | 2355 | AGTCATGTGTTAAAATCGA |
| siRNA 614 | 614 | CGATTTTAACACATGACTC | 2356 | GAGTCATGTGTTAAAATCG |
| siRNA 615 | 615 | GATTTTAACACATGACTCA | 2357 | TGAGTCATGTGTTAAAATC |
| siRNA 616 | 616 | ATTTTAACACATGACTCAA | 2358 | TTGAGTCATGTGTTAAAAT |
| siRNA 617 | 617 | TTTTAACACATGACTCAAG | 2359 | CTTGAGTCATGTGTTAAAA |
| siRNA 618 | 618 | TTTAACACATGACTCAAGT | 2360 | ACTTGAGTCATGTGTTAAA |
| siRNA 619 | 619 | TTAACACATGACTCAAGTA | 2361 | TACTTGAGTCATGTGTTAA |
| siRNA 620 | 620 | TAACACATGACTCAAGTAT | 2362 | ATACTTGAGTCATGTGTTA |
| siRNA 621 | 621 | AACACATGACTCAAGTATT | 2363 | AATACTTGAGTCATGTGTT |
| siRNA 622 | 622 | ACACATGACTCAAGTATTC | 2364 | GAATACTTGAGTCATGTGT |
| siRNA 623 | 623 | CACATGACTCAAGTATTCG | 2365 | CGAATACTTGAGTCATGTG |
| siRNA 624 | 624 | ACATGACTCAAGTATTCGA | 2366 | TCGAATACTTGAGTCATGT |
| siRNA 625 | 625 | CATGACTCAAGTATTCGAT | 2367 | ATCGAATACTTGAGTCATG |
| siRNA 626 | 626 | ATGACTCAAGTATTCGATA | 2368 | TATCGAATACTTGAGTCAT |
| siRNA 627 | 627 | TGACTCAAGTATTCGATAT | 2369 | ATATCGAATACTTGAGTCA |
| siRNA 628 | 628 | GACTCAAGTATTCGATATT | 2370 | AATATCGAATACTTGAGTC |
| siRNA 629 | 629 | ACTCAAGTATTCGATATTT | 2371 | AAATATCGAATACTTGAGT |
| siRNA 630 | 630 | CTCAAGTATTCGATATTTG | 2372 | CAAATATCGAATACTTGAG |
| siRNA 631 | 631 | TCAAGTATTCGATATTTGC | 2373 | GCAAATATCGAATACTTGA |
| siRNA 632 | 632 | CAAGTATTCGATATTTGCA | 2374 | TGCAAATATCGAATACTTG |
| siRNA 633 | 633 | AAGTATTCGATATTTGCAG | 2375 | CTGCAAATATCGAATACTT |
| siRNA 634 | 634 | AGTATTCGATATTTGCAGG | 2376 | CCTGCAAATATCGAATACT |
| siRNA 635 | 635 | GTATTCGATATTTGCAGGA | 2377 | TCCTGCAAATATCGAATAC |
| siRNA 636 | 636 | TATTCGATATTTGCAGGAA | 2378 | TTCCTGCAAATATCGAATA |
| siRNA 637 | 637 | ATTCGATATTTGCAGGAAA | 2379 | TTTCCTGCAAATATCGAAT |
| siRNA 638 | 638 | TTCGATATTTGCAGGAAAT | 2380 | ATTTCCTGCAAATATCGAA |
| siRNA 639 | 639 | TCGATATTTGCAGGAAATA | 2381 | TATTTCCTGCAAATATCGA |
| siRNA 640 | 640 | CGATATTTGCAGGAAATAT | 2382 | ATATTTCCTGCAAATATCG |
| siRNA 641 | 641 | GATATTTGCAGGAAATATA | 2383 | TATATTTCCTGCAAATATC |
| siRNA 642 | 642 | ATATTTGCAGGAAATATAT | 2384 | ATATATTTCCTGCAAATAT |
| siRNA 643 | 643 | TATTTGCAGGAAATATATA | 2385 | TATATATTTCCTGCAAATA |
| siRNA 644 | 644 | ATTTGCAGGAAATATATAA | 2386 | TTATATATTTCCTGCAAAT |
| siRNA 645 | 645 | TTTGCAGGAAATATATAAT | 2387 | ATTATATATTTCCTGCAAA |
| siRNA 646 | 646 | TTGCAGGAAATATATAATT | 2388 | AATTATATATTTCCTGCAA |
| siRNA 647 | 647 | TGCAGGAAATATATAATTC | 2389 | GAATTATATATTTCCTGCA |
| siRNA 648 | 648 | GCAGGAAATATATAATTCA | 2390 | TGAATTATATATTTCCTGC |
| siRNA 649 | 649 | CAGGAAATATATAATTCAA | 2391 | TTGAATTATATATTTCCTG |
| siRNA 650 | 650 | AGGAAATATATAATTCAAA | 2392 | TTTGAATTATATATTTCCT |
| siRNA 651 | 651 | GGAAATATATAATTCAAAT | 2393 | ATTTGAATTATATATTTCC |
| siRNA 652 | 652 | GAAATATATAATTCAAATA | 2394 | TATTTGAATTATATATTTC |
| siRNA 653 | 653 | AAATATATAATTCAAATAA | 2395 | TTATTTGAATTATATATTT |
| siRNA 654 | 654 | AATATATAATTCAAATAAT | 2396 | ATTATTTGAATTATATATT |
| siRNA 655 | 655 | ATATATAATTCAAATAATC | 2397 | GATTATTTGAATTATATAT |
| siRNA 656 | 656 | TATATAATTCAAATAATCA | 2398 | TGATTATTTGAATTATATA |
| siRNA 657 | 657 | ATATAATTCAAATAATCAA | 2399 | TTGATTATTTGAATTATAT |
| siRNA 658 | 658 | TATAATTCAAATAATCAAA | 2400 | TTTGATTATTTGAATTATA |
| siRNA 659 | 659 | ATAATTCAAATAATCAAAA | 2401 | TTTTGATTATTTGAATTAT |
| siRNA 660 | 660 | TAATTCAAATAATCAAAAG | 2402 | CTTTTGATTATTTGAATTA |
| siRNA 661 | 661 | AATTCAAATAATCAAAAGA | 2403 | TCTTTTGATTATTTGAATT |
| siRNA 662 | 662 | ATTCAAATAATCAAAAGAT | 2404 | ATCTTTTGATTATTTGAAT |
| siRNA 663 | 663 | TTCAAATAATCAAAAGATT | 2405 | AATCTTTTGATTATTTGAA |
| siRNA 664 | 664 | TCAAATAATCAAAAGATTG | 2406 | CAATCTTTTGATTATTTGA |
| siRNA 665 | 665 | CAAATAATCAAAAGATTGT | 2407 | ACAATCTTTTGATTATTTG |
| siRNA 666 | 666 | AAATAATCAAAAGATTGTT | 2408 | AACAATCTTTTGATTATTT |
| siRNA 667 | 667 | AATAATCAAAAGATTGTTA | 2409 | TAACAATCTTTTGATTATT |
| siRNA 668 | 668 | ATAATCAAAAGATTGTTAA | 2410 | TTAACAATCTTTTGATTAT |
| siRNA 669 | 669 | TAATCAAAAGATTGTTAAC | 2411 | GTTAACAATCTTTTGATTA |
| siRNA 670 | 670 | AATCAAAAGATTGTTAACC | 2412 | GGTTAACAATCTTTTGATT |
| siRNA 671 | 671 | ATCAAAAGATTGTTAACCT | 2413 | AGGTTAACAATCTTTTGAT |
| siRNA 672 | 672 | TCAAAAGATTGTTAACCTG | 2414 | CAGGTTAACAATCTTTTGA |
| siRNA 673 | 673 | CAAAAGATTGTTAACCTGA | 2415 | TCAGGTTAACAATCTTTTG |
| siRNA 674 | 674 | AAAAGATTGTTAACCTGAA | 2416 | TTCAGGTTAACAATCTTTT |
| siRNA 675 | 675 | AAAGATTGTTAACCTGAAA | 2417 | TTTCAGGTTAACAATCTTT |
| siRNA 676 | 676 | AAGATTGTTAACCTGAAAG | 2418 | CTTTCAGGTTAACAATCTT |
| siRNA 677 | 677 | AGATTGTTAACCTGAAAGA | 2419 | TCTTTCAGGTTAACAATCT |
| siRNA 678 | 678 | GATTGTTAACCTGAAAGAG | 2420 | CTCTTTCAGGTTAACAATC |
| siRNA 679 | 679 | ATTGTTAACCTGAAAGAGA | 2421 | TCTCTTTCAGGTTAACAAT |
| siRNA 680 | 680 | TTGTTAACCTGAAAGAGAA | 2422 | TTCTCTTTCAGGTTAACAA |
| siRNA 681 | 681 | TGTTAACCTGAAAGAGAAG | 2423 | CTTCTCTTTCAGGTTAACA |
| siRNA 682 | 682 | GTTAACCTGAAAGAGAAGG | 2424 | CCTTCTCTTTCAGGTTAAC |
| siRNA 683 | 683 | TTAACCTGAAAGAGAAGGT | 2425 | ACCTTCTCTTTCAGGTTAA |
| siRNA 684 | 684 | TAACCTGAAAGAGAAGGTA | 2426 | TACCTTCTCTTTCAGGTTA |
| siRNA 685 | 685 | AACCTGAAAGAGAAGGTAG | 2427 | CTACCTTCTCTTTCAGGTT |
| siRNA 686 | 686 | ACCTGAAAGAGAAGGTAGC | 2428 | GCTACCTTCTCTTTCAGGT |
| siRNA 687 | 687 | CCTGAAAGAGAAGGTAGCC | 2429 | GGCTACCTTCTCTTTCAGG |
| siRNA 688 | 688 | CTGAAAGAGAAGGTAGCCC | 2430 | GGGCTACCTTCTCTTTCAG |
| siRNA 689 | 689 | TGAAAGAGAAGGTAGCCCA | 2431 | TGGGCTACCTTCTCTTTCA |
| siRNA 690 | 690 | GAAAGAGAAGGTAGCCCAG | 2432 | CTGGGCTACCTTCTCTTTC |
| siRNA 691 | 691 | AAAGAGAAGGTAGCCCAGC | 2433 | GCTGGGCTACCTTCTCTTT |
| siRNA 692 | 692 | AAGAGAAGGTAGCCCAGCT | 2434 | AGCTGGGCTACCTTCTCTT |
| siRNA 693 | 693 | AGAGAAGGTAGCCCAGCTT | 2435 | AAGCTGGGCTACCTTCTCT |
| siRNA 694 | 694 | GAGAAGGTAGCCCAGCTTG | 2436 | CAAGCTGGGCTACCTTCTC |
| siRNA 695 | 695 | AGAAGGTAGCCCAGCTTGA | 2437 | TCAAGCTGGGCTACCTTCT |
| siRNA 696 | 696 | GAAGGTAGCCCAGCTTGAA | 2438 | TTCAAGCTGGGCTACCTTC |
| siRNA 697 | 697 | AAGGTAGCCCAGCTTGAAG | 2439 | CTTCAAGCTGGGCTACCTT |
| siRNA 698 | 698 | AGGTAGCCCAGCTTGAAGC | 2440 | GCTTCAAGCTGGGCTACCT |
| siRNA 699 | 699 | GGTAGCCCAGCTTGAAGCA | 2441 | TGCTTCAAGCTGGGCTACC |
| siRNA 700 | 700 | GTAGCCCAGCTTGAAGCAC | 2442 | GTGCTTCAAGCTGGGCTAC |
| siRNA 701 | 701 | TAGCCCAGCTTGAAGCACA | 2443 | TGTGCTTCAAGCTGGGCTA |
| siRNA 702 | 702 | AGCCCAGCTTGAAGCACAG | 2444 | CTGTGCTTCAAGCTGGGCT |
| siRNA 703 | 703 | GCCCAGCTTGAAGCACAGT | 2445 | ACTGTGCTTCAAGCTGGGC |
| siRNA 704 | 704 | CCCAGCTTGAAGCACAGTG | 2446 | CACTGTGCTTCAAGCTGGG |
| siRNA 705 | 705 | CCAGCTTGAAGCACAGTGC | 2447 | GCACTGTGCTTCAAGCTGG |
| siRNA 706 | 706 | CAGCTTGAAGCACAGTGCC | 2448 | GGCACTGTGCTTCAAGCTG |
| siRNA 707 | 707 | AGCTTGAAGCACAGTGCCA | 2449 | TGGCACTGTGCTTCAAGCT |
| siRNA 708 | 708 | GCTTGAAGCACAGTGCCAG | 2450 | CTGGCACTGTGCTTCAAGC |
| siRNA 709 | 709 | CTTGAAGCACAGTGCCAGG | 2451 | CCTGGCACTGTGCTTCAAG |
| siRNA 710 | 710 | TTGAAGCACAGTGCCAGGA | 2452 | TCCTGGCACTGTGCTTCAA |
| siRNA 711 | 711 | TGAAGCACAGTGCCAGGAA | 2453 | TTCCTGGCACTGTGCTTCA |
| siRNA 712 | 712 | GAAGCACAGTGCCAGGAAC | 2454 | GTTCCTGGCACTGTGCTTC |
| siRNA 713 | 713 | AAGCACAGTGCCAGGAACC | 2455 | GGTTCCTGGCACTGTGCTT |
| siRNA 714 | 714 | AGCACAGTGCCAGGAACCT | 2456 | AGGTTCCTGGCACTGTGCT |
| siRNA 715 | 715 | GCACAGTGCCAGGAACCTT | 2457 | AAGGTTCCTGGCACTGTGC |
| siRNA 716 | 716 | CACAGTGCCAGGAACCTTG | 2458 | CAAGGTTCCTGGCACTGTG |
| siRNA 717 | 717 | ACAGTGCCAGGAACCTTGC | 2459 | GCAAGGTTCCTGGCACTGT |
| siRNA 718 | 718 | CAGTGCCAGGAACCTTGCA | 2460 | TGCAAGGTTCCTGGCACTG |
| siRNA 719 | 719 | AGTGCCAGGAACCTTGCAA | 2461 | TTGCAAGGTTCCTGGCACT |
| siRNA 720 | 720 | GTGCCAGGAACCTTGCAAA | 2462 | TTTGCAAGGTTCCTGGCAC |
| siRNA 721 | 721 | TGCCAGGAACCTTGCAAAG | 2463 | CTTTGCAAGGTTCCTGGCA |
| siRNA 722 | 722 | GCCAGGAACCTTGCAAAGA | 2464 | TCTTTGCAAGGTTCCTGGC |
| siRNA 723 | 723 | CCAGGAACCTTGCAAAGAC | 2465 | GTCTTTGCAAGGTTCCTGG |
| siRNA 724 | 724 | CAGGAACCTTGCAAAGACA | 2466 | TGTCTTTGCAAGGTTCCTG |
| siRNA 725 | 725 | AGGAACCTTGCAAAGACAC | 2467 | GTGTCTTTGCAAGGTTCCT |
| siRNA 726 | 726 | GGAACCTTGCAAAGACACG | 2468 | CGTGTCTTTGCAAGGTTCC |
| siRNA 727 | 727 | GAACCTTGCAAAGACACGG | 2469 | CCGTGTCTTTGCAAGGTTC |
| siRNA 728 | 728 | AACCTTGCAAAGACACGGT | 2470 | ACCGTGTCTTTGCAAGGTT |
| siRNA 729 | 729 | ACCTTGCAAAGACACGGTG | 2471 | CACCGTGTCTTTGCAAGGT |
| siRNA 730 | 730 | CCTTGCAAAGACACGGTGC | 2472 | GCACCGTGTCTTTGCAAGG |
| siRNA 731 | 731 | CTTGCAAAGACACGGTGCA | 2473 | TGCACCGTGTCTTTGCAAG |
| siRNA 732 | 732 | TTGCAAAGACACGGTGCAA | 2474 | TTGCACCGTGTCTTTGCAA |
| siRNA 733 | 733 | TGCAAAGACACGGTGCAAA | 2475 | TTTGCACCGTGTCTTTGCA |
| siRNA 734 | 734 | GCAAAGACACGGTGCAAAT | 2476 | ATTTGCACCGTGTCTTTGC |
| siRNA 735 | 735 | CAAAGACACGGTGCAAATC | 2477 | GATTTGCACCGTGTCTTTG |
| siRNA 736 | 736 | AAAGACACGGTGCAAATCC | 2478 | GGATTTGCACCGTGTCTTT |
| siRNA 737 | 737 | AAGACACGGTGCAAATCCA | 2479 | TGGATTTGCACCGTGTCTT |
| siRNA 738 | 738 | AGACACGGTGCAAATCCAT | 2480 | ATGGATTTGCACCGTGTCT |
| siRNA 739 | 739 | GACACGGTGCAAATCCATG | 2481 | CATGGATTTGCACCGTGTC |
| siRNA 740 | 740 | ACACGGTGCAAATCCATGA | 2482 | TCATGGATTTGCACCGTGT |
| siRNA 741 | 741 | CACGGTGCAAATCCATGAT | 2483 | ATCATGGATTTGCACCGTG |
| siRNA 742 | 742 | ACGGTGCAAATCCATGATA | 2484 | TATCATGGATTTGCACCGT |
| siRNA 743 | 743 | CGGTGCAAATCCATGATAT | 2485 | ATATCATGGATTTGCACCG |
| siRNA 744 | 744 | GGTGCAAATCCATGATATC | 2486 | GATATCATGGATTTGCACC |
| siRNA 745 | 745 | GTGCAAATCCATGATATCA | 2487 | TGATATCATGGATTTGCAC |
| siRNA 746 | 746 | TGCAAATCCATGATATCAC | 2488 | GTGATATCATGGATTTGCA |
| siRNA 747 | 747 | GCAAATCCATGATATCACT | 2489 | AGTGATATCATGGATTTGC |
| siRNA 748 | 748 | CAAATCCATGATATCACTG | 2490 | CAGTGATATCATGGATTTG |
| siRNA 749 | 749 | AAATCCATGATATCACTGG | 2491 | CCAGTGATATCATGGATTT |
| siRNA 750 | 750 | AATCCATGATATCACTGGG | 2492 | CCCAGTGATATCATGGATT |
| siRNA 751 | 751 | ATCCATGATATCACTGGGA | 2493 | TCCCAGTGATATCATGGAT |
| siRNA 752 | 752 | TCCATGATATCACTGGGAA | 2494 | TTCCCAGTGATATCATGGA |
| siRNA 753 | 753 | CCATGATATCACTGGGAAA | 2495 | TTTCCCAGTGATATCATGG |
| siRNA 754 | 754 | CATGATATCACTGGGAAAG | 2496 | CTTTCCCAGTGATATCATG |
| siRNA 755 | 755 | ATGATATCACTGGGAAAGA | 2497 | TCTTTCCCAGTGATATCAT |
| siRNA 756 | 756 | TGATATCACTGGGAAAGAT | 2498 | ATCTTTCCCAGTGATATCA |
| siRNA 757 | 757 | GATATCACTGGGAAAGATT | 2499 | AATCTTTCCCAGTGATATC |
| siRNA 758 | 758 | ATATCACTGGGAAAGATTG | 2500 | CAATCTTTCCCAGTGATAT |
| siRNA 759 | 759 | TATCACTGGGAAAGATTGT | 2501 | ACAATCTTTCCCAGTGATA |
| siRNA 760 | 760 | ATCACTGGGAAAGATTGTC | 2502 | GACAATCTTTCCCAGTGAT |
| siRNA 761 | 761 | TCACTGGGAAAGATTGTCA | 2503 | TGACAATCTTTCCCAGTGA |
| siRNA 762 | 762 | CACTGGGAAAGATTGTCAA | 2504 | TTGACAATCTTTCCCAGTG |
| siRNA 763 | 763 | ACTGGGAAAGATTGTCAAG | 2505 | CTTGACAATCTTTCCCAGT |
| siRNA 764 | 764 | CTGGGAAAGATTGTCAAGA | 2506 | TCTTGACAATCTTTCCCAG |
| siRNA 765 | 765 | TGGGAAAGATTGTCAAGAC | 2507 | GTCTTGACAATCTTTCCCA |
| siRNA 766 | 766 | GGGAAAGATTGTCAAGACA | 2508 | TGTCTTGACAATCTTTCCC |
| siRNA 767 | 767 | GGAAAGATTGTCAAGACAT | 2509 | ATGTCTTGACAATCTTTCC |
| siRNA 768 | 768 | GAAAGATTGTCAAGACATT | 2510 | AATGTCTTGACAATCTTTC |
| siRNA 769 | 769 | AAAGATTGTCAAGACATTG | 2511 | CAATGTCTTGACAATCTTT |
| siRNA 770 | 770 | AAGATTGTCAAGACATTGC | 2512 | GCAATGTCTTGACAATCTT |
| siRNA 771 | 771 | AGATTGTCAAGACATTGCC | 2513 | GGCAATGTCTTGACAATCT |
| siRNA 772 | 772 | GATTGTCAAGACATTGCCA | 2514 | TGGCAATGTCTTGACAATC |
| siRNA 773 | 773 | ATTGTCAAGACATTGCCAA | 2515 | TTGGCAATGTCTTGACAAT |
| siRNA 774 | 774 | TTGTCAAGACATTGCCAAT | 2516 | ATTGGCAATGTCTTGACAA |
| siRNA 775 | 775 | TGTCAAGACATTGCCAATA | 2517 | TATTGGCAATGTCTTGACA |
| siRNA 776 | 776 | GTCAAGACATTGCCAATAA | 2518 | TTATTGGCAATGTCTTGAC |
| siRNA 777 | 777 | TCAAGACATTGCCAATAAG | 2519 | CTTATTGGCAATGTCTTGA |
| siRNA 778 | 778 | CAAGACATTGCCAATAAGG | 2520 | CCTTATTGGCAATGTCTTG |
| siRNA 779 | 779 | AAGACATTGCCAATAAGGG | 2521 | CCCTTATTGGCAATGTCTT |
| siRNA 780 | 780 | AGACATTGCCAATAAGGGA | 2522 | TCCCTTATTGGCAATGTCT |
| siRNA 781 | 781 | GACATTGCCAATAAGGGAG | 2523 | CTCCCTTATTGGCAATGTC |
| siRNA 782 | 782 | ACATTGCCAATAAGGGAGC | 2524 | GCTCCCTTATTGGCAATGT |
| siRNA 783 | 783 | CATTGCCAATAAGGGAGCT | 2525 | AGCTCCCTTATTGGCAATG |
| siRNA 784 | 784 | ATTGCCAATAAGGGAGCTA | 2526 | TAGCTCCCTTATTGGCAAT |
| siRNA 785 | 785 | TTGCCAATAAGGGAGCTAA | 2527 | TTAGCTCCCTTATTGGCAA |
| siRNA 786 | 786 | TGCCAATAAGGGAGCTAAA | 2528 | TTTAGCTCCCTTATTGGCA |
| siRNA 787 | 787 | GCCAATAAGGGAGCTAAAC | 2529 | GTTTAGCTCCCTTATTGGC |
| siRNA 788 | 788 | CCAATAAGGGAGCTAAACA | 2530 | TGTTTAGCTCCCTTATTGG |
| siRNA 789 | 789 | CAATAAGGGAGCTAAACAG | 2531 | CTGTTTAGCTCCCTTATTG |
| siRNA 790 | 790 | AATAAGGGAGCTAAACAGA | 2532 | TCTGTTTAGCTCCCTTATT |
| siRNA 791 | 791 | ATAAGGGAGCTAAACAGAG | 2533 | CTCTGTTTAGCTCCCTTAT |
| siRNA 792 | 792 | TAAGGGAGCTAAACAGAGC | 2534 | GCTCTGTTTAGCTCCCTTA |
| siRNA 793 | 793 | AAGGGAGCTAAACAGAGCG | 2535 | CGCTCTGTTTAGCTCCCTT |
| siRNA 794 | 794 | AGGGAGCTAAACAGAGCGG | 2536 | CCGCTCTGTTTAGCTCCCT |
| siRNA 795 | 795 | GGGAGCTAAACAGAGCGGG | 2537 | CCCGCTCTGTTTAGCTCCC |
| siRNA 796 | 796 | GGAGCTAAACAGAGCGGGC | 2538 | GCCCGCTCTGTTTAGCTCC |
| siRNA 797 | 797 | GAGCTAAACAGAGCGGGCT | 2539 | AGCCCGCTCTGTTTAGCTC |
| siRNA 798 | 798 | AGCTAAACAGAGCGGGCTT | 2540 | AAGCCCGCTCTGTTTAGCT |
| siRNA 799 | 799 | GCTAAACAGAGCGGGCTTT | 2541 | AAAGCCCGCTCTGTTTAGC |
| siRNA 800 | 800 | CTAAACAGAGCGGGCTTTA | 2542 | TAAAGCCCGCTCTGTTTAG |
| siRNA 801 | 801 | TAAACAGAGCGGGCTTTAC | 2543 | GTAAAGCCCGCTCTGTTTA |
| siRNA 802 | 802 | AAACAGAGCGGGCTTTACT | 2544 | AGTAAAGCCCGCTCTGTTT |
| siRNA 803 | 803 | AACAGAGCGGGCTTTACTT | 2545 | AAGTAAAGCCCGCTCTGTT |
| siRNA 804 | 804 | ACAGAGCGGGCTTTACTTT | 2546 | AAAGTAAAGCCCGCTCTGT |
| siRNA 805 | 805 | CAGAGCGGGCTTTACTTTA | 2547 | TAAAGTAAAGCCCGCTCTG |
| siRNA 806 | 806 | AGAGCGGGCTTTACTTTAT | 2548 | ATAAAGTAAAGCCCGCTCT |
| siRNA 807 | 807 | GAGCGGGCTTTACTTTATT | 2549 | AATAAAGTAAAGCCCGCTC |
| siRNA 808 | 808 | AGCGGGCTTTACTTTATTA | 2550 | TAATAAAGTAAAGCCCGCT |
| siRNA 809 | 809 | GCGGGCTTTACTTTATTAA | 2551 | TTAATAAAGTAAAGCCCGC |
| siRNA 810 | 810 | CGGGCTTTACTTTATTAAA | 2552 | TTTAATAAAGTAAAGCCCG |
| siRNA 811 | 811 | GGGCTTTACTTTATTAAAC | 2553 | GTTTAATAAAGTAAAGCCC |
| siRNA 812 | 812 | GGCTTTACTTTATTAAACC | 2554 | GGTTTAATAAAGTAAAGCC |
| siRNA 813 | 813 | GCTTTACTTTATTAAACCT | 2555 | AGGTTTAATAAAGTAAAGC |
| siRNA 814 | 814 | CTTTACTTTATTAAACCTC | 2556 | GAGGTTTAATAAAGTAAAG |
| siRNA 815 | 815 | TTTACTTTATTAAACCTCT | 2557 | AGAGGTTTAATAAAGTAAA |
| siRNA 816 | 816 | TTACTTTATTAAACCTCTG | 2558 | CAGAGGTTTAATAAAGTAA |
| siRNA 817 | 817 | TACTTTATTAAACCTCTGA | 2559 | TCAGAGGTTTAATAAAGTA |
| siRNA 818 | 818 | ACTTTATTAAACCTCTGAA | 2560 | TTCAGAGGTTTAATAAAGT |
| siRNA 819 | 819 | CTTTATTAAACCTCTGAAA | 2561 | TTTCAGAGGTTTAATAAAG |
| siRNA 820 | 820 | TTTATTAAACCTCTGAAAG | 2562 | CTTTCAGAGGTTTAATAAA |
| siRNA 821 | 821 | TTATTAAACCTCTGAAAGC | 2563 | GCTTTCAGAGGTTTAATAA |
| siRNA 822 | 822 | TATTAAACCTCTGAAAGCT | 2564 | AGCTTTCAGAGGTTTAATA |
| siRNA 823 | 823 | ATTAAACCTCTGAAAGCTA | 2565 | TAGCTTTCAGAGGTTTAAT |
| siRNA 824 | 824 | TTAAACCTCTGAAAGCTAA | 2566 | TTAGCTTTCAGAGGTTTAA |
| siRNA 825 | 825 | TAAACCTCTGAAAGCTAAC | 2567 | GTTAGCTTTCAGAGGTTTA |
| siRNA 826 | 826 | AAACCTCTGAAAGCTAACC | 2568 | GGTTAGCTTTCAGAGGTTT |
| siRNA 827 | 827 | AACCTCTGAAAGCTAACCA | 2569 | TGGTTAGCTTTCAGAGGTT |
| siRNA 828 | 828 | ACCTCTGAAAGCTAACCAG | 2570 | CTGGTTAGCTTTCAGAGGT |
| siRNA 829 | 829 | CCTCTGAAAGCTAACCAGC | 2571 | GCTGGTTAGCTTTCAGAGG |
| siRNA 830 | 830 | CTCTGAAAGCTAACCAGCA | 2572 | TGCTGGTTAGCTTTCAGAG |
| siRNA 831 | 831 | TCTGAAAGCTAACCAGCAA | 2573 | TTGCTGGTTAGCTTTCAGA |
| siRNA 832 | 832 | CTGAAAGCTAACCAGCAAT | 2574 | ATTGCTGGTTAGCTTTCAG |
| siRNA 833 | 833 | TGAAAGCTAACCAGCAATT | 2575 | AATTGCTGGTTAGCTTTCA |
| siRNA 834 | 834 | GAAAGCTAACCAGCAATTC | 2576 | GAATTGCTGGTTAGCTTTC |
| siRNA 835 | 835 | AAAGCTAACCAGCAATTCT | 2577 | AGAATTGCTGGTTAGCTTT |
| siRNA 836 | 836 | AAGCTAACCAGCAATTCTT | 2578 | AAGAATTGCTGGTTAGCTT |
| siRNA 837 | 837 | AGCTAACCAGCAATTCTTA | 2579 | TAAGAATTGCTGGTTAGCT |
| siRNA 838 | 838 | GCTAACCAGCAATTCTTAG | 2580 | CTAAGAATTGCTGGTTAGC |
| siRNA 839 | 839 | CTAACCAGCAATTCTTAGT | 2581 | ACTAAGAATTGCTGGTTAG |
| siRNA 840 | 840 | TAACCAGCAATTCTTAGTC | 2582 | GACTAAGAATTGCTGGTTA |
| siRNA 841 | 841 | AACCAGCAATTCTTAGTCT | 2583 | AGACTAAGAATTGCTGGTT |
| siRNA 842 | 842 | ACCAGCAATTCTTAGTCTA | 2584 | TAGACTAAGAATTGCTGGT |
| siRNA 843 | 843 | CCAGCAATTCTTAGTCTAC | 2585 | GTAGACTAAGAATTGCTGG |
| siRNA 844 | 844 | CAGCAATTCTTAGTCTACT | 2586 | AGTAGACTAAGAATTGCTG |
| siRNA 845 | 845 | AGCAATTCTTAGTCTACTG | 2587 | CAGTAGACTAAGAATTGCT |
| siRNA 846 | 846 | GCAATTCTTAGTCTACTGT | 2588 | ACAGTAGACTAAGAATTGC |
| siRNA 847 | 847 | CAATTCTTAGTCTACTGTG | 2589 | CACAGTAGACTAAGAATTG |
| siRNA 848 | 848 | AATTCTTAGTCTACTGTGA | 2590 | TCACAGTAGACTAAGAATT |
| siRNA 849 | 849 | ATTCTTAGTCTACTGTGAA | 2591 | TTCACAGTAGACTAAGAAT |
| siRNA 850 | 850 | TTCTTAGTCTACTGTGAAA | 2592 | TTTCACAGTAGACTAAGAA |
| siRNA 851 | 851 | TCTTAGTCTACTGTGAAAT | 2593 | ATTTCACAGTAGACTAAGA |
| siRNA 852 | 852 | CTTAGTCTACTGTGAAATC | 2594 | GATTTCACAGTAGACTAAG |
| siRNA 853 | 853 | TTAGTCTACTGTGAAATCG | 2595 | CGATTTCACAGTAGACTAA |
| siRNA 854 | 854 | TAGTCTACTGTGAAATCGA | 2596 | TCGATTTCACAGTAGACTA |
| siRNA 855 | 855 | AGTCTACTGTGAAATCGAT | 2597 | ATCGATTTCACAGTAGACT |
| siRNA 856 | 856 | GTCTACTGTGAAATCGATG | 2598 | CATCGATTTCACAGTAGAC |
| siRNA 857 | 857 | TCTACTGTGAAATCGATGG | 2599 | CCATCGATTTCACAGTAGA |
| siRNA 858 | 858 | CTACTGTGAAATCGATGGG | 2600 | CCCATCGATTTCACAGTAG |
| siRNA 859 | 859 | TACTGTGAAATCGATGGGT | 2601 | ACCCATCGATTTCACAGTA |
| siRNA 860 | 860 | ACTGTGAAATCGATGGGTC | 2602 | GACCCATCGATTTCACAGT |
| siRNA 861 | 861 | CTGTGAAATCGATGGGTCT | 2603 | AGACCCATCGATTTCACAG |
| siRNA 862 | 862 | TGTGAAATCGATGGGTCTG | 2604 | CAGACCCATCGATTTCACA |
| siRNA 863 | 863 | GTGAAATCGATGGGTCTGG | 2605 | CCAGACCCATCGATTTCAC |
| siRNA 864 | 864 | TGAAATCGATGGGTCTGGA | 2606 | TCCAGACCCATCGATTTCA |
| siRNA 865 | 865 | GAAATCGATGGGTCTGGAA | 2607 | TTCCAGACCCATCGATTTC |
| siRNA 866 | 866 | AAATCGATGGGTCTGGAAA | 2608 | TTTCCAGACCCATCGATTT |
| siRNA 867 | 867 | AATCGATGGGTCTGGAAAT | 2609 | ATTTCCAGACCCATCGATT |
| siRNA 868 | 868 | ATCGATGGGTCTGGAAATG | 2610 | CATTTCCAGACCCATCGAT |
| siRNA 869 | 869 | TCGATGGGTCTGGAAATGG | 2611 | CCATTTCCAGACCCATCGA |
| siRNA 870 | 870 | CGATGGGTCTGGAAATGGA | 2612 | TCCATTTCCAGACCCATCG |
| siRNA 871 | 871 | GATGGGTCTGGAAATGGAT | 2613 | ATCCATTTCCAGACCCATC |
| siRNA 872 | 872 | ATGGGTCTGGAAATGGATG | 2614 | CATCCATTTCCAGACCCAT |
| siRNA 873 | 873 | TGGGTCTGGAAATGGATGG | 2615 | CCATCCATTTCCAGACCCA |
| siRNA 874 | 874 | GGGTCTGGAAATGGATGGA | 2616 | TCCATCCATTTCCAGACCC |
| siRNA 875 | 875 | GGTCTGGAAATGGATGGAC | 2617 | GTCCATCCATTTCCAGACC |
| siRNA 876 | 876 | GTCTGGAAATGGATGGACT | 2618 | AGTCCATCCATTTCCAGAC |
| siRNA 877 | 877 | TCTGGAAATGGATGGACTG | 2619 | CAGTCCATCCATTTCCAGA |
| siRNA 878 | 878 | CTGGAAATGGATGGACTGT | 2620 | ACAGTCCATCCATTTCCAG |
| siRNA 879 | 879 | TGGAAATGGATGGACTGTG | 2621 | CACAGTCCATCCATTTCCA |
| siRNA 880 | 880 | GGAAATGGATGGACTGTGT | 2622 | ACACAGTCCATCCATTTCC |
| siRNA 881 | 881 | GAAATGGATGGACTGTGTT | 2623 | AACACAGTCCATCCATTTC |
| siRNA 882 | 882 | AAATGGATGGACTGTGTTT | 2624 | AAACACAGTCCATCCATTT |
| siRNA 883 | 883 | AATGGATGGACTGTGTTTC | 2625 | GAAACACAGTCCATCCATT |
| siRNA 884 | 884 | ATGGATGGACTGTGTTTCA | 2626 | TGAAACACAGTCCATCCAT |
| siRNA 885 | 885 | TGGATGGACTGTGTTTCAG | 2627 | CTGAAACACAGTCCATCCA |
| siRNA 886 | 886 | GGATGGACTGTGTTTCAGA | 2628 | TCTGAAACACAGTCCATCC |
| siRNA 887 | 887 | GATGGACTGTGTTTCAGAA | 2629 | TTCTGAAACACAGTCCATC |
| siRNA 888 | 888 | ATGGACTGTGTTTCAGAAG | 2630 | CTTCTGAAACACAGTCCAT |
| siRNA 889 | 889 | TGGACTGTGTTTCAGAAGA | 2631 | TCTTCTGAAACACAGTCCA |
| siRNA 890 | 890 | GGACTGTGTTTCAGAAGAG | 2632 | CTCTTCTGAAACACAGTCC |
| siRNA 891 | 891 | GACTGTGTTTCAGAAGAGA | 2633 | TCTCTTCTGAAACACAGTC |
| siRNA 892 | 892 | ACTGTGTTTCAGAAGAGAC | 2634 | GTCTCTTCTGAAACACAGT |
| siRNA 893 | 893 | CTGTGTTTCAGAAGAGACT | 2635 | AGTCTCTTCTGAAACACAG |
| siRNA 894 | 894 | TGTGTTTCAGAAGAGACTT | 2636 | AAGTCTCTTCTGAAACACA |
| siRNA 895 | 895 | GTGTTTCAGAAGAGACTTG | 2637 | CAAGTCTCTTCTGAAACAC |
| siRNA 896 | 896 | TGTTTCAGAAGAGACTTGA | 2638 | TCAAGTCTCTTCTGAAACA |
| siRNA 897 | 897 | GTTTCAGAAGAGACTTGAT | 2639 | ATCAAGTCTCTTCTGAAAC |
| siRNA 898 | 898 | TTTCAGAAGAGACTTGATG | 2640 | CATCAAGTCTCTTCTGAAA |
| siRNA 899 | 899 | TTCAGAAGAGACTTGATGG | 2641 | CCATCAAGTCTCTTCTGAA |
| siRNA 900 | 900 | TCAGAAGAGACTTGATGGC | 2642 | GCCATCAAGTCTCTTCTGA |
| siRNA 901 | 901 | CAGAAGAGACTTGATGGCA | 2643 | TGCCATCAAGTCTCTTCTG |
| siRNA 902 | 902 | AGAAGAGACTTGATGGCAG | 2644 | CTGCCATCAAGTCTCTTCT |
| siRNA 903 | 903 | GAAGAGACTTGATGGCAGT | 2645 | ACTGCCATCAAGTCTCTTC |
| siRNA 904 | 904 | AAGAGACTTGATGGCAGTG | 2646 | CACTGCCATCAAGTCTCTT |
| siRNA 905 | 905 | AGAGACTTGATGGCAGTGT | 2647 | ACACTGCCATCAAGTCTCT |
| siRNA 906 | 906 | GAGACTTGATGGCAGTGTA | 2648 | TACACTGCCATCAAGTCTC |
| siRNA 907 | 907 | AGACTTGATGGCAGTGTAG | 2649 | CTACACTGCCATCAAGTCT |
| siRNA 908 | 908 | GACTTGATGGCAGTGTAGA | 2650 | TCTACACTGCCATCAAGTC |
| siRNA 909 | 909 | ACTTGATGGCAGTGTAGAT | 2651 | ATCTACACTGCCATCAAGT |
| siRNA 910 | 910 | CTTGATGGCAGTGTAGATT | 2652 | AATCTACACTGCCATCAAG |
| siRNA 911 | 911 | TTGATGGCAGTGTAGATTT | 2653 | AAATCTACACTGCCATCAA |
| siRNA 912 | 912 | TGATGGCAGTGTAGATTTC | 2654 | GAAATCTACACTGCCATCA |
| siRNA 913 | 913 | GATGGCAGTGTAGATTTCA | 2655 | TGAAATCTACACTGCCATC |
| siRNA 914 | 914 | ATGGCAGTGTAGATTTCAA | 2656 | TTGAAATCTACACTGCCAT |
| siRNA 915 | 915 | TGGCAGTGTAGATTTCAAG | 2657 | CTTGAAATCTACACTGCCA |
| siRNA 916 | 916 | GGCAGTGTAGATTTCAAGA | 2658 | TCTTGAAATCTACACTGCC |
| siRNA 917 | 917 | GCAGTGTAGATTTCAAGAA | 2659 | TTCTTGAAATCTACACTGC |
| siRNA 918 | 918 | CAGTGTAGATTTCAAGAAA | 2660 | TTTCTTGAAATCTACACTG |
| siRNA 919 | 919 | AGTGTAGATTTCAAGAAAA | 2661 | TTTTCTTGAAATCTACACT |
| siRNA 920 | 920 | GTGTAGATTTCAAGAAAAA | 2662 | TTTTTCTTGAAATCTACAC |
| siRNA 921 | 921 | TGTAGATTTCAAGAAAAAC | 2663 | GTTTTTCTTGAAATCTACA |
| siRNA 922 | 922 | GTAGATTTCAAGAAAAACT | 2664 | AGTTTTTCTTGAAATCTAC |
| siRNA 923 | 923 | TAGATTTCAAGAAAAACTG | 2665 | CAGTTTTTCTTGAAATCTA |
| siRNA 924 | 924 | AGATTTCAAGAAAAACTGG | 2666 | CCAGTTTTTCTTGAAATCT |
| siRNA 925 | 925 | GATTTCAAGAAAAACTGGA | 2667 | TCCAGTTTTTCTTGAAATC |
| siRNA 926 | 926 | ATTTCAAGAAAAACTGGAT | 2668 | ATCCAGTTTTTCTTGAAAT |
| siRNA 927 | 927 | TTTCAAGAAAAACTGGATT | 2669 | AATCCAGTTTTTCTTGAAA |
| siRNA 928 | 928 | TTCAAGAAAAACTGGATTC | 2670 | GAATCCAGTTTTTCTTGAA |
| siRNA 929 | 929 | TCAAGAAAAACTGGATTCA | 2671 | TGAATCCAGTTTTTCTTGA |
| siRNA 930 | 930 | CAAGAAAAACTGGATTCAA | 2672 | TTGAATCCAGTTTTTCTTG |
| siRNA 931 | 931 | AAGAAAAACTGGATTCAAT | 2673 | ATTGAATCCAGTTTTTCTT |
| siRNA 932 | 932 | AGAAAAACTGGATTCAATA | 2674 | TATTGAATCCAGTTTTTCT |
| siRNA 933 | 933 | GAAAAACTGGATTCAATAT | 2675 | ATATTGAATCCAGTTTTTC |
| siRNA 934 | 934 | AAAAACTGGATTCAATATA | 2676 | TATATTGAATCCAGTTTTT |
| siRNA 935 | 935 | AAAACTGGATTCAATATAA | 2677 | TTATATTGAATCCAGTTTT |
| siRNA 936 | 936 | AAACTGGATTCAATATAAA | 2678 | TTTATATTGAATCCAGTTT |
| siRNA 937 | 937 | AACTGGATTCAATATAAAG | 2679 | CTTTATATTGAATCCAGTT |
| siRNA 938 | 938 | ACTGGATTCAATATAAAGA | 2680 | TCTTTATATTGAATCCAGT |
| siRNA 939 | 939 | CTGGATTCAATATAAAGAA | 2681 | TTCTTTATATTGAATCCAG |
| siRNA 940 | 940 | TGGATTCAATATAAAGAAG | 2682 | CTTCTTTATATTGAATCCA |
| siRNA 941 | 941 | GGATTCAATATAAAGAAGG | 2683 | CCTTCTTTATATTGAATCC |
| siRNA 942 | 942 | GATTCAATATAAAGAAGGA | 2684 | TCCTTCTTTATATTGAATC |
| siRNA 943 | 943 | ATTCAATATAAAGAAGGAT | 2685 | ATCCTTCTTTATATTGAAT |
| siRNA 944 | 944 | TTCAATATAAAGAAGGATT | 2686 | AATCCTTCTTTATATTGAA |
| siRNA 945 | 945 | TCAATATAAAGAAGGATTT | 2687 | AAATCCTTCTTTATATTGA |
| siRNA 946 | 946 | CAATATAAAGAAGGATTTG | 2688 | CAAATCCTTCTTTATATTG |
| siRNA 947 | 947 | AATATAAAGAAGGATTTGG | 2689 | CCAAATCCTTCTTTATATT |
| siRNA 948 | 948 | ATATAAAGAAGGATTTGGA | 2690 | TCCAAATCCTTCTTTATAT |
| siRNA 949 | 949 | TATAAAGAAGGATTTGGAC | 2691 | GTCCAAATCCTTCTTTATA |
| siRNA 950 | 950 | ATAAAGAAGGATTTGGACA | 2692 | TGTCCAAATCCTTCTTTAT |
| siRNA 951 | 951 | TAAAGAAGGATTTGGACAT | 2693 | ATGTCCAAATCCTTCTTTA |
| siRNA 952 | 952 | AAAGAAGGATTTGGACATC | 2694 | GATGTCCAAATCCTTCTTT |
| siRNA 953 | 953 | AAGAAGGATTTGGACATCT | 2695 | AGATGTCCAAATCCTTCTT |
| siRNA 954 | 954 | AGAAGGATTTGGACATCTG | 2696 | CAGATGTCCAAATCCTTCT |
| siRNA 955 | 955 | GAAGGATTTGGACATCTGT | 2697 | ACAGATGTCCAAATCCTTC |
| siRNA 956 | 956 | AAGGATTTGGACATCTGTC | 2698 | GACAGATGTCCAAATCCTT |
| siRNA 957 | 957 | AGGATTTGGACATCTGTCT | 2699 | AGACAGATGTCCAAATCCT |
| siRNA 958 | 958 | GGATTTGGACATCTGTCTC | 2700 | GAGACAGATGTCCAAATCC |
| siRNA 959 | 959 | GATTTGGACATCTGTCTCC | 2701 | GGAGACAGATGTCCAAATC |
| siRNA 960 | 960 | ATTTGGACATCTGTCTCCT | 2702 | AGGAGACAGATGTCCAAAT |
| siRNA 961 | 961 | TTTGGACATCTGTCTCCTA | 2703 | TAGGAGACAGATGTCCAAA |
| siRNA 962 | 962 | TTGGACATCTGTCTCCTAC | 2704 | GTAGGAGACAGATGTCCAA |
| siRNA 963 | 963 | TGGACATCTGTCTCCTACT | 2705 | AGTAGGAGACAGATGTCCA |
| siRNA 964 | 964 | GGACATCTGTCTCCTACTG | 2706 | CAGTAGGAGACAGATGTCC |
| siRNA 965 | 965 | GACATCTGTCTCCTACTGG | 2707 | CCAGTAGGAGACAGATGTC |
| siRNA 966 | 966 | ACATCTGTCTCCTACTGGC | 2708 | GCCAGTAGGAGACAGATGT |
| siRNA 967 | 967 | CATCTGTCTCCTACTGGCA | 2709 | TGCCAGTAGGAGACAGATG |
| siRNA 968 | 968 | ATCTGTCTCCTACTGGCAC | 2710 | GTGCCAGTAGGAGACAGAT |
| siRNA 969 | 969 | TCTGTCTCCTACTGGCACA | 2711 | TGTGCCAGTAGGAGACAGA |
| siRNA 970 | 970 | CTGTCTCCTACTGGCACAA | 2712 | TTGTGCCAGTAGGAGACAG |
| siRNA 971 | 971 | TGTCTCCTACTGGCACAAC | 2713 | GTTGTGCCAGTAGGAGACA |
| siRNA 972 | 972 | GTCTCCTACTGGCACAACA | 2714 | TGTTGTGCCAGTAGGAGAC |
| siRNA 973 | 973 | TCTCCTACTGGCACAACAG | 2715 | CTGTTGTGCCAGTAGGAGA |
| siRNA 974 | 974 | CTCCTACTGGCACAACAGA | 2716 | TCTGTTGTGCCAGTAGGAG |
| siRNA 975 | 975 | TCCTACTGGCACAACAGAA | 2717 | TTCTGTTGTGCCAGTAGGA |
| siRNA 976 | 976 | CCTACTGGCACAACAGAAT | 2718 | ATTCTGTTGTGCCAGTAGG |
| siRNA 977 | 977 | CTACTGGCACAACAGAATT | 2719 | AATTCTGTTGTGCCAGTAG |
| siRNA 978 | 978 | TACTGGCACAACAGAATTT | 2720 | AAATTCTGTTGTGCCAGTA |
| siRNA 979 | 979 | ACTGGCACAACAGAATTTT | 2721 | AAAATTCTGTTGTGCCAGT |
| siRNA 980 | 980 | CTGGCACAACAGAATTTTG | 2722 | CAAAATTCTGTTGTGCCAG |
| siRNA 981 | 981 | TGGCACAACAGAATTTTGG | 2723 | CCAAAATTCTGTTGTGCCA |
| siRNA 982 | 982 | GGCACAACAGAATTTTGGC | 2724 | GCCAAAATTCTGTTGTGCC |
| siRNA 983 | 983 | GCACAACAGAATTTTGGCT | 2725 | AGCCAAAATTCTGTTGTGC |
| siRNA 984 | 984 | CACAACAGAATTTTGGCTG | 2726 | CAGCCAAAATTCTGTTGTG |
| siRNA 985 | 985 | ACAACAGAATTTTGGCTGG | 2727 | CCAGCCAAAATTCTGTTGT |
| siRNA 986 | 986 | CAACAGAATTTTGGCTGGG | 2728 | CCCAGCCAAAATTCTGTTG |
| siRNA 987 | 987 | AACAGAATTTTGGCTGGGA | 2729 | TCCCAGCCAAAATTCTGTT |
| siRNA 988 | 988 | ACAGAATTTTGGCTGGGAA | 2730 | TTCCCAGCCAAAATTCTGT |
| siRNA 989 | 989 | CAGAATTTTGGCTGGGAAA | 2731 | TTTCCCAGCCAAAATTCTG |
| siRNA 990 | 990 | AGAATTTTGGCTGGGAAAT | 2732 | ATTTCCCAGCCAAAATTCT |
| siRNA 991 | 991 | GAATTTTGGCTGGGAAATG | 2733 | CATTTCCCAGCCAAAATTC |
| siRNA 992 | 992 | AATTTTGGCTGGGAAATGA | 2734 | TCATTTCCCAGCCAAAATT |
| siRNA 993 | 993 | ATTTTGGCTGGGAAATGAG | 2735 | CTCATTTCCCAGCCAAAAT |
| siRNA 994 | 994 | TTTTGGCTGGGAAATGAGA | 2736 | TCTCATTTCCCAGCCAAAA |
| siRNA 995 | 995 | TTTGGCTGGGAAATGAGAA | 2737 | TTCTCATTTCCCAGCCAAA |
| siRNA 996 | 996 | TTGGCTGGGAAATGAGAAG | 2738 | CTTCTCATTTCCCAGCCAA |
| siRNA 997 | 997 | TGGCTGGGAAATGAGAAGA | 2739 | TCTTCTCATTTCCCAGCCA |
| siRNA 998 | 998 | GGCTGGGAAATGAGAAGAT | 2740 | ATCTTCTCATTTCCCAGCC |
| siRNA 999 | 999 | GCTGGGAAATGAGAAGATT | 2741 | AATCTTCTCATTTCCCAGC |
| siRNA 1000 | 1000 | CTGGGAAATGAGAAGATTC | 2742 | GAATCTTCTCATTTCCCAG |
| siRNA 1001 | 1001 | TGGGAAATGAGAAGATTCA | 2743 | TGAATCTTCTCATTTCCCA |
| siRNA 1002 | 1002 | GGGAAATGAGAAGATTCAT | 2744 | ATGAATCTTCTCATTTCCC |
| siRNA 1003 | 1003 | GGAAATGAGAAGATTCATT | 2745 | AATGAATCTTCTCATTTCC |
| siRNA 1004 | 1004 | GAAATGAGAAGATTCATTT | 2746 | AAATGAATCTTCTCATTTC |
| siRNA 1005 | 1005 | AAATGAGAAGATTCATTTG | 2747 | CAAATGAATCTTCTCATTT |
| siRNA 1006 | 1006 | AATGAGAAGATTCATTTGA | 2748 | TCAAATGAATCTTCTCATT |
| siRNA 1007 | 1007 | ATGAGAAGATTCATTTGAT | 2749 | ATCAAATGAATCTTCTCAT |
| siRNA 1008 | 1008 | TGAGAAGATTCATTTGATA | 2750 | TATCAAATGAATCTTCTCA |
| siRNA 1009 | 1009 | GAGAAGATTCATTTGATAA | 2751 | TTATCAAATGAATCTTCTC |
| siRNA 1010 | 1010 | AGAAGATTCATTTGATAAG | 2752 | CTTATCAAATGAATCTTCT |
| siRNA 1011 | 1011 | GAAGATTCATTTGATAAGC | 2753 | GCTTATCAAATGAATCTTC |
| siRNA 1012 | 1012 | AAGATTCATTTGATAAGCA | 2754 | TGCTTATCAAATGAATCTT |
| siRNA 1013 | 1013 | AGATTCATTTGATAAGCAC | 2755 | GTGCTTATCAAATGAATCT |
| siRNA 1014 | 1014 | GATTCATTTGATAAGCACA | 2756 | TGTGCTTATCAAATGAATC |
| siRNA 1015 | 1015 | ATTCATTTGATAAGCACAC | 2757 | GTGTGCTTATCAAATGAAT |
| siRNA 1016 | 1016 | TTCATTTGATAAGCACACA | 2758 | TGTGTGCTTATCAAATGAA |
| siRNA 1017 | 1017 | TCATTTGATAAGCACACAG | 2759 | CTGTGTGCTTATCAAATGA |
| siRNA 1018 | 1018 | CATTTGATAAGCACACAGT | 2760 | ACTGTGTGCTTATCAAATG |
| siRNA 1019 | 1019 | ATTTGATAAGCACACAGTC | 2761 | GACTGTGTGCTTATCAAAT |
| siRNA 1020 | 1020 | TTTGATAAGCACACAGTCT | 2762 | AGACTGTGTGCTTATCAAA |
| siRNA 1021 | 1021 | TTGATAAGCACACAGTCTG | 2763 | CAGACTGTGTGCTTATCAA |
| siRNA 1022 | 1022 | TGATAAGCACACAGTCTGC | 2764 | GCAGACTGTGTGCTTATCA |
| siRNA 1023 | 1023 | GATAAGCACACAGTCTGCC | 2765 | GGCAGACTGTGTGCTTATC |
| siRNA 1024 | 1024 | ATAAGCACACAGTCTGCCA | 2766 | TGGCAGACTGTGTGCTTAT |
| siRNA 1025 | 1025 | TAAGCACACAGTCTGCCAT | 2767 | ATGGCAGACTGTGTGCTTA |
| siRNA 1026 | 1026 | AAGCACACAGTCTGCCATC | 2768 | GATGGCAGACTGTGTGCTT |
| siRNA 1027 | 1027 | AGCACACAGTCTGCCATCC | 2769 | GGATGGCAGACTGTGTGCT |
| siRNA 1028 | 1028 | GCACACAGTCTGCCATCCC | 2770 | GGGATGGCAGACTGTGTGC |
| siRNA 1029 | 1029 | CACACAGTCTGCCATCCCA | 2771 | TGGGATGGCAGACTGTGTG |
| siRNA 1030 | 1030 | ACACAGTCTGCCATCCCAT | 2772 | ATGGGATGGCAGACTGTGT |
| siRNA 1031 | 1031 | CACAGTCTGCCATCCCATA | 2773 | TATGGGATGGCAGACTGTG |
| siRNA 1032 | 1032 | ACAGTCTGCCATCCCATAT | 2774 | ATATGGGATGGCAGACTGT |
| siRNA 1033 | 1033 | CAGTCTGCCATCCCATATG | 2775 | CATATGGGATGGCAGACTG |
| siRNA 1034 | 1034 | AGTCTGCCATCCCATATGC | 2776 | GCATATGGGATGGCAGACT |
| siRNA 1035 | 1035 | GTCTGCCATCCCATATGCA | 2777 | TGCATATGGGATGGCAGAC |
| siRNA 1036 | 1036 | TCTGCCATCCCATATGCAT | 2778 | ATGCATATGGGATGGCAGA |
| siRNA 1037 | 1037 | CTGCCATCCCATATGCATT | 2779 | AATGCATATGGGATGGCAG |
| siRNA 1038 | 1038 | TGCCATCCCATATGCATTA | 2780 | TAATGCATATGGGATGGCA |
| siRNA 1039 | 1039 | GCCATCCCATATGCATTAA | 2781 | TTAATGCATATGGGATGGC |
| siRNA 1040 | 1040 | CCATCCCATATGCATTAAG | 2782 | CTTAATGCATATGGGATGG |
| siRNA 1041 | 1041 | CATCCCATATGCATTAAGA | 2783 | TCTTAATGCATATGGGATG |
| siRNA 1042 | 1042 | ATCCCATATGCATTAAGAG | 2784 | CTCTTAATGCATATGGGAT |
| siRNA 1043 | 1043 | TCCCATATGCATTAAGAGT | 2785 | ACTCTTAATGCATATGGGA |
| siRNA 1044 | 1044 | CCCATATGCATTAAGAGTG | 2786 | CACTCTTAATGCATATGGG |
| siRNA 1045 | 1045 | CCATATGCATTAAGAGTGG | 2787 | CCACTCTTAATGCATATGG |
| siRNA 1046 | 1046 | CATATGCATTAAGAGTGGA | 2788 | TCCACTCTTAATGCATATG |
| siRNA 1047 | 1047 | ATATGCATTAAGAGTGGAA | 2789 | TTCCACTCTTAATGCATAT |
| siRNA 1048 | 1048 | TATGCATTAAGAGTGGAAC | 2790 | GTTCCACTCTTAATGCATA |
| siRNA 1049 | 1049 | ATGCATTAAGAGTGGAACT | 2791 | AGTTCCACTCTTAATGCAT |
| siRNA 1050 | 1050 | TGCATTAAGAGTGGAACTG | 2792 | CAGTTCCACTCTTAATGCA |
| siRNA 1051 | 1051 | GCATTAAGAGTGGAACTGG | 2793 | CCAGTTCCACTCTTAATGC |
| siRNA 1052 | 1052 | CATTAAGAGTGGAACTGGA | 2794 | TCCAGTTCCACTCTTAATG |
| siRNA 1053 | 1053 | ATTAAGAGTGGAACTGGAA | 2795 | TTCCAGTTCCACTCTTAAT |
| siRNA 1054 | 1054 | TTAAGAGTGGAACTGGAAG | 2796 | CTTCCAGTTCCACTCTTAA |
| siRNA 1055 | 1055 | TAAGAGTGGAACTGGAAGA | 2797 | TCTTCCAGTTCCACTCTTA |
| siRNA 1056 | 1056 | AAGAGTGGAACTGGAAGAC | 2798 | GTCTTCCAGTTCCACTCTT |
| siRNA 1057 | 1057 | AGAGTGGAACTGGAAGACT | 2799 | AGTCTTCCAGTTCCACTCT |
| siRNA 1058 | 1058 | GAGTGGAACTGGAAGACTG | 2800 | CAGTCTTCCAGTTCCACTC |
| siRNA 1059 | 1059 | AGTGGAACTGGAAGACTGG | 2801 | CCAGTCTTCCAGTTCCACT |
| siRNA 1060 | 1060 | GTGGAACTGGAAGACTGGA | 2802 | TCCAGTCTTCCAGTTCCAC |
| siRNA 1061 | 1061 | TGGAACTGGAAGACTGGAA | 2803 | TTCCAGTCTTCCAGTTCCA |
| siRNA 1062 | 1062 | GGAACTGGAAGACTGGAAT | 2804 | ATTCCAGTCTTCCAGTTCC |
| siRNA 1063 | 1063 | GAACTGGAAGACTGGAATG | 2805 | CATTCCAGTCTTCCAGTTC |
| siRNA 1064 | 1064 | AACTGGAAGACTGGAATGG | 2806 | CCATTCCAGTCTTCCAGTT |
| siRNA 1065 | 1065 | ACTGGAAGACTGGAATGGC | 2807 | GCCATTCCAGTCTTCCAGT |
| siRNA 1066 | 1066 | CTGGAAGACTGGAATGGCA | 2808 | TGCCATTCCAGTCTTCCAG |
| siRNA 1067 | 1067 | TGGAAGACTGGAATGGCAG | 2809 | CTGCCATTCCAGTCTTCCA |
| siRNA 1068 | 1068 | GGAAGACTGGAATGGCAGA | 2810 | TCTGCCATTCCAGTCTTCC |
| siRNA 1069 | 1069 | GAAGACTGGAATGGCAGAA | 2811 | TTCTGCCATTCCAGTCTTC |
| siRNA 1070 | 1070 | AAGACTGGAATGGCAGAAC | 2812 | GTTCTGCCATTCCAGTCTT |
| siRNA 1071 | 1071 | AGACTGGAATGGCAGAACC | 2813 | GGTTCTGCCATTCCAGTCT |
| siRNA 1072 | 1072 | GACTGGAATGGCAGAACCA | 2814 | TGGTTCTGCCATTCCAGTC |
| siRNA 1073 | 1073 | ACTGGAATGGCAGAACCAG | 2815 | CTGGTTCTGCCATTCCAGT |
| siRNA 1074 | 1074 | CTGGAATGGCAGAACCAGT | 2816 | ACTGGTTCTGCCATTCCAG |
| siRNA 1075 | 1075 | TGGAATGGCAGAACCAGTA | 2817 | TACTGGTTCTGCCATTCCA |
| siRNA 1076 | 1076 | GGAATGGCAGAACCAGTAC | 2818 | GTACTGGTTCTGCCATTCC |
| siRNA 1077 | 1077 | GAATGGCAGAACCAGTACT | 2819 | AGTACTGGTTCTGCCATTC |
| siRNA 1078 | 1078 | AATGGCAGAACCAGTACTG | 2820 | CAGTACTGGTTCTGCCATT |
| siRNA 1079 | 1079 | ATGGCAGAACCAGTACTGC | 2821 | GCAGTACTGGTTCTGCCAT |
| siRNA 1080 | 1080 | TGGCAGAACCAGTACTGCA | 2822 | TGCAGTACTGGTTCTGCCA |
| siRNA 1081 | 1081 | GGCAGAACCAGTACTGCAG | 2823 | CTGCAGTACTGGTTCTGCC |
| siRNA 1082 | 1082 | GCAGAACCAGTACTGCAGA | 2824 | TCTGCAGTACTGGTTCTGC |
| siRNA 1083 | 1083 | CAGAACCAGTACTGCAGAC | 2825 | GTCTGCAGTACTGGTTCTG |
| siRNA 1084 | 1084 | AGAACCAGTACTGCAGACT | 2826 | AGTCTGCAGTACTGGTTCT |
| siRNA 1085 | 1085 | GAACCAGTACTGCAGACTA | 2827 | TAGTCTGCAGTACTGGTTC |
| siRNA 1086 | 1086 | AACCAGTACTGCAGACTAT | 2828 | ATAGTCTGCAGTACTGGTT |
| siRNA 1087 | 1087 | ACCAGTACTGCAGACTATG | 2829 | CATAGTCTGCAGTACTGGT |
| siRNA 1088 | 1088 | CCAGTACTGCAGACTATGC | 2830 | GCATAGTCTGCAGTACTGG |
| siRNA 1089 | 1089 | CAGTACTGCAGACTATGCC | 2831 | GGCATAGTCTGCAGTACTG |
| siRNA 1090 | 1090 | AGTACTGCAGACTATGCCA | 2832 | TGGCATAGTCTGCAGTACT |
| siRNA 1091 | 1091 | GTACTGCAGACTATGCCAT | 2833 | ATGGCATAGTCTGCAGTAC |
| siRNA 1092 | 1092 | TACTGCAGACTATGCCATG | 2834 | CATGGCATAGTCTGCAGTA |
| siRNA 1093 | 1093 | ACTGCAGACTATGCCATGT | 2835 | ACATGGCATAGTCTGCAGT |
| siRNA 1094 | 1094 | CTGCAGACTATGCCATGTT | 2836 | AACATGGCATAGTCTGCAG |
| siRNA 1095 | 1095 | TGCAGACTATGCCATGTTC | 2837 | GAACATGGCATAGTCTGCA |
| siRNA 1096 | 1096 | GCAGACTATGCCATGTTCA | 2838 | TGAACATGGCATAGTCTGC |
| siRNA 1097 | 1097 | CAGACTATGCCATGTTCAA | 2839 | TTGAACATGGCATAGTCTG |
| siRNA 1098 | 1098 | AGACTATGCCATGTTCAAG | 2840 | CTTGAACATGGCATAGTCT |
| siRNA 1099 | 1099 | GACTATGCCATGTTCAAGG | 2841 | CCTTGAACATGGCATAGTC |
| siRNA 1100 | 1100 | ACTATGCCATGTTCAAGGT | 2842 | ACCTTGAACATGGCATAGT |
| siRNA 1101 | 1101 | CTATGCCATGTTCAAGGTG | 2843 | CACCTTGAACATGGCATAG |
| siRNA 1102 | 1102 | TATGCCATGTTCAAGGTGG | 2844 | CCACCTTGAACATGGCATA |
| siRNA 1103 | 1103 | ATGCCATGTTCAAGGTGGG | 2845 | CCCACCTTGAACATGGCAT |
| siRNA 1104 | 1104 | TGCCATGTTCAAGGTGGGA | 2846 | TCCCACCTTGAACATGGCA |
| siRNA 1105 | 1105 | GCCATGTTCAAGGTGGGAC | 2847 | GTCCCACCTTGAACATGGC |
| siRNA 1106 | 1106 | CCATGTTCAAGGTGGGACC | 2848 | GGTCCCACCTTGAACATGG |
| siRNA 1107 | 1107 | CATGTTCAAGGTGGGACCT | 2849 | AGGTCCCACCTTGAACATG |
| siRNA 1108 | 1108 | ATGTTCAAGGTGGGACCTG | 2850 | CAGGTCCCACCTTGAACAT |
| siRNA 1109 | 1109 | TGTTCAAGGTGGGACCTGA | 2851 | TCAGGTCCCACCTTGAACA |
| siRNA 1110 | 1110 | GTTCAAGGTGGGACCTGAA | 2852 | TTCAGGTCCCACCTTGAAC |
| siRNA 1111 | 1111 | TTCAAGGTGGGACCTGAAG | 2853 | CTTCAGGTCCCACCTTGAA |
| siRNA 1112 | 1112 | TCAAGGTGGGACCTGAAGC | 2854 | GCTTCAGGTCCCACCTTGA |
| siRNA 1113 | 1113 | CAAGGTGGGACCTGAAGCT | 2855 | AGCTTCAGGTCCCACCTTG |
| siRNA 1114 | 1114 | AAGGTGGGACCTGAAGCTG | 2856 | CAGCTTCAGGTCCCACCTT |
| siRNA 1115 | 1115 | AGGTGGGACCTGAAGCTGA | 2857 | TCAGCTTCAGGTCCCACCT |
| siRNA 1116 | 1116 | GGTGGGACCTGAAGCTGAC | 2858 | GTCAGCTTCAGGTCCCACC |
| siRNA 1117 | 1117 | GTGGGACCTGAAGCTGACA | 2859 | TGTCAGCTTCAGGTCCCAC |
| siRNA 1118 | 1118 | TGGGACCTGAAGCTGACAA | 2860 | TTGTCAGCTTCAGGTCCCA |
| siRNA 1119 | 1119 | GGGACCTGAAGCTGACAAG | 2861 | CTTGTCAGCTTCAGGTCCC |
| siRNA 1120 | 1120 | GGACCTGAAGCTGACAAGT | 2862 | ACTTGTCAGCTTCAGGTCC |
| siRNA 1121 | 1121 | GACCTGAAGCTGACAAGTA | 2863 | TACTTGTCAGCTTCAGGTC |
| siRNA 1122 | 1122 | ACCTGAAGCTGACAAGTAC | 2864 | GTACTTGTCAGCTTCAGGT |
| siRNA 1123 | 1123 | CCTGAAGCTGACAAGTACC | 2865 | GGTACTTGTCAGCTTCAGG |
| siRNA 1124 | 1124 | CTGAAGCTGACAAGTACCG | 2866 | CGGTACTTGTCAGCTTCAG |
| siRNA 1125 | 1125 | TGAAGCTGACAAGTACCGC | 2867 | GCGGTACTTGTCAGCTTCA |
| siRNA 1126 | 1126 | GAAGCTGACAAGTACCGCC | 2868 | GGCGGTACTTGTCAGCTTC |
| siRNA 1127 | 1127 | AAGCTGACAAGTACCGCCT | 2869 | AGGCGGTACTTGTCAGCTT |
| siRNA 1128 | 1128 | AGCTGACAAGTACCGCCTA | 2870 | TAGGCGGTACTTGTCAGCT |
| siRNA 1129 | 1129 | GCTGACAAGTACCGCCTAA | 2871 | TTAGGCGGTACTTGTCAGC |
| siRNA 1130 | 1130 | CTGACAAGTACCGCCTAAC | 2872 | GTTAGGCGGTACTTGTCAG |
| siRNA 1131 | 1131 | TGACAAGTACCGCCTAACA | 2873 | TGTTAGGCGGTACTTGTCA |
| siRNA 1132 | 1132 | GACAAGTACCGCCTAACAT | 2874 | ATGTTAGGCGGTACTTGTC |
| siRNA 1133 | 1133 | ACAAGTACCGCCTAACATA | 2875 | TATGTTAGGCGGTACTTGT |
| siRNA 1134 | 1134 | CAAGTACCGCCTAACATAT | 2876 | ATATGTTAGGCGGTACTTG |
| siRNA 1135 | 1135 | AAGTACCGCCTAACATATG | 2877 | CATATGTTAGGCGGTACTT |
| siRNA 1136 | 1136 | AGTACCGCCTAACATATGC | 2878 | GCATATGTTAGGCGGTACT |
| siRNA 1137 | 1137 | GTACCGCCTAACATATGCC | 2879 | GGCATATGTTAGGCGGTAC |
| siRNA 1138 | 1138 | TACCGCCTAACATATGCCT | 2880 | AGGCATATGTTAGGCGGTA |
| siRNA 1139 | 1139 | ACCGCCTAACATATGCCTA | 2881 | TAGGCATATGTTAGGCGGT |
| siRNA 1140 | 1140 | CCGCCTAACATATGCCTAC | 2882 | GTAGGCATATGTTAGGCGG |
| siRNA 1141 | 1141 | CGCCTAACATATGCCTACT | 2883 | AGTAGGCATATGTTAGGCG |
| siRNA 1142 | 1142 | GCCTAACATATGCCTACTT | 2884 | AAGTAGGCATATGTTAGGC |
| siRNA 1143 | 1143 | CCTAACATATGCCTACTTC | 2885 | GAAGTAGGCATATGTTAGG |
| siRNA 1144 | 1144 | CTAACATATGCCTACTTCG | 2886 | CGAAGTAGGCATATGTTAG |
| siRNA 1145 | 1145 | TAACATATGCCTACTTCGC | 2887 | GCGAAGTAGGCATATGTTA |
| siRNA 1146 | 1146 | AACATATGCCTACTTCGCT | 2888 | AGCGAAGTAGGCATATGTT |
| siRNA 1147 | 1147 | ACATATGCCTACTTCGCTG | 2889 | CAGCGAAGTAGGCATATGT |
| siRNA 1148 | 1148 | CATATGCCTACTTCGCTGG | 2890 | CCAGCGAAGTAGGCATATG |
| siRNA 1149 | 1149 | ATATGCCTACTTCGCTGGT | 2891 | ACCAGCGAAGTAGGCATAT |
| siRNA 1150 | 1150 | TATGCCTACTTCGCTGGTG | 2892 | CACCAGCGAAGTAGGCATA |
| siRNA 1151 | 1151 | ATGCCTACTTCGCTGGTGG | 2893 | CCACCAGCGAAGTAGGCAT |
| siRNA 1152 | 1152 | TGCCTACTTCGCTGGTGGG | 2894 | CCCACCAGCGAAGTAGGCA |
| siRNA 1153 | 1153 | GCCTACTTCGCTGGTGGGG | 2895 | CCCCACCAGCGAAGTAGGC |
| siRNA 1154 | 1154 | CCTACTTCGCTGGTGGGGA | 2896 | TCCCCACCAGCGAAGTAGG |
| siRNA 1155 | 1155 | CTACTTCGCTGGTGGGGAT | 2897 | ATCCCCACCAGCGAAGTAG |
| siRNA 1156 | 1156 | TACTTCGCTGGTGGGGATG | 2898 | CATCCCCACCAGCGAAGTA |
| siRNA 1157 | 1157 | ACTTCGCTGGTGGGGATGC | 2899 | GCATCCCCACCAGCGAAGT |
| siRNA 1158 | 1158 | CTTCGCTGGTGGGGATGCT | 2900 | AGCATCCCCACCAGCGAAG |
| siRNA 1159 | 1159 | TTCGCTGGTGGGGATGCTG | 2901 | CAGCATCCCCACCAGCGAA |
| siRNA 1160 | 1160 | TCGCTGGTGGGGATGCTGG | 2902 | CCAGCATCCCCACCAGCGA |
| siRNA 1161 | 1161 | CGCTGGTGGGGATGCTGGA | 2903 | TCCAGCATCCCCACCAGCG |
| siRNA 1162 | 1162 | GCTGGTGGGGATGCTGGAG | 2904 | CTCCAGCATCCCCACCAGC |
| siRNA 1163 | 1163 | CTGGTGGGGATGCTGGAGA | 2905 | TCTCCAGCATCCCCACCAG |
| siRNA 1164 | 1164 | TGGTGGGGATGCTGGAGAT | 2906 | ATCTCCAGCATCCCCACCA |
| siRNA 1165 | 1165 | GGTGGGGATGCTGGAGATG | 2907 | CATCTCCAGCATCCCCACC |
| siRNA 1166 | 1166 | GTGGGGATGCTGGAGATGC | 2908 | GCATCTCCAGCATCCCCAC |
| siRNA 1167 | 1167 | TGGGGATGCTGGAGATGCC | 2909 | GGCATCTCCAGCATCCCCA |
| siRNA 1168 | 1168 | GGGGATGCTGGAGATGCCT | 2910 | AGGCATCTCCAGCATCCCC |
| siRNA 1169 | 1169 | GGGATGCTGGAGATGCCTT | 2911 | AAGGCATCTCCAGCATCCC |
| siRNA 1170 | 1170 | GGATGCTGGAGATGCCTTT | 2912 | AAAGGCATCTCCAGCATCC |
| siRNA 1171 | 1171 | GATGCTGGAGATGCCTTTG | 2913 | CAAAGGCATCTCCAGCATC |
| siRNA 1172 | 1172 | ATGCTGGAGATGCCTTTGA | 2914 | TCAAAGGCATCTCCAGCAT |
| siRNA 1173 | 1173 | TGCTGGAGATGCCTTTGAT | 2915 | ATCAAAGGCATCTCCAGCA |
| siRNA 1174 | 1174 | GCTGGAGATGCCTTTGATG | 2916 | CATCAAAGGCATCTCCAGC |
| siRNA 1175 | 1175 | CTGGAGATGCCTTTGATGG | 2917 | CCATCAAAGGCATCTCCAG |
| siRNA 1176 | 1176 | TGGAGATGCCTTTGATGGC | 2918 | GCCATCAAAGGCATCTCCA |
| siRNA 1177 | 1177 | GGAGATGCCTTTGATGGCT | 2919 | AGCCATCAAAGGCATCTCC |
| siRNA 1178 | 1178 | GAGATGCCTTTGATGGCTT | 2920 | AAGCCATCAAAGGCATCTC |
| siRNA 1179 | 1179 | AGATGCCTTTGATGGCTTT | 2921 | AAAGCCATCAAAGGCATCT |
| siRNA 1180 | 1180 | GATGCCTTTGATGGCTTTG | 2922 | CAAAGCCATCAAAGGCATC |
| siRNA 1181 | 1181 | ATGCCTTTGATGGCTTTGA | 2923 | TCAAAGCCATCAAAGGCAT |
| siRNA 1182 | 1182 | TGCCTTTGATGGCTTTGAT | 2924 | ATCAAAGCCATCAAAGGCA |
| siRNA 1183 | 1183 | GCCTTTGATGGCTTTGATT | 2925 | AATCAAAGCCATCAAAGGC |
| siRNA 1184 | 1184 | CCTTTGATGGCTTTGATTT | 2926 | AAATCAAAGCCATCAAAGG |
| siRNA 1185 | 1185 | CTTTGATGGCTTTGATTTT | 2927 | AAAATCAAAGCCATCAAAG |
| siRNA 1186 | 1186 | TTTGATGGCTTTGATTTTG | 2928 | CAAAATCAAAGCCATCAAA |
| siRNA 1187 | 1187 | TTGATGGCTTTGATTTTGG | 2929 | CCAAAATCAAAGCCATCAA |
| siRNA 1188 | 1188 | TGATGGCTTTGATTTTGGC | 2930 | GCCAAAATCAAAGCCATCA |
| siRNA 1189 | 1189 | GATGGCTTTGATTTTGGCG | 2931 | CGCCAAAATCAAAGCCATC |
| siRNA 1190 | 1190 | ATGGCTTTGATTTTGGCGA | 2932 | TCGCCAAAATCAAAGCCAT |
| siRNA 1191 | 1191 | TGGCTTTGATTTTGGCGAT | 2933 | ATCGCCAAAATCAAAGCCA |
| siRNA 1192 | 1192 | GGCTTTGATTTTGGCGATG | 2934 | CATCGCCAAAATCAAAGCC |
| siRNA 1193 | 1193 | GCTTTGATTTTGGCGATGA | 2935 | TCATCGCCAAAATCAAAGC |
| siRNA 1194 | 1194 | CTTTGATTTTGGCGATGAT | 2936 | ATCATCGCCAAAATCAAAG |
| siRNA 1195 | 1195 | TTTGATTTTGGCGATGATC | 2937 | GATCATCGCCAAAATCAAA |
| siRNA 1196 | 1196 | TTGATTTTGGCGATGATCC | 2938 | GGATCATCGCCAAAATCAA |
| siRNA 1197 | 1197 | TGATTTTGGCGATGATCCT | 2939 | AGGATCATCGCCAAAATCA |
| siRNA 1198 | 1198 | GATTTTGGCGATGATCCTA | 2940 | TAGGATCATCGCCAAAATC |
| siRNA 1199 | 1199 | ATTTTGGCGATGATCCTAG | 2941 | CTAGGATCATCGCCAAAAT |
| siRNA 1200 | 1200 | TTTTGGCGATGATCCTAGT | 2942 | ACTAGGATCATCGCCAAAA |
| siRNA 1201 | 1201 | TTTGGCGATGATCCTAGTG | 2943 | CACTAGGATCATCGCCAAA |
| siRNA 1202 | 1202 | TTGGCGATGATCCTAGTGA | 2944 | TCACTAGGATCATCGCCAA |
| siRNA 1203 | 1203 | TGGCGATGATCCTAGTGAC | 2945 | GTCACTAGGATCATCGCCA |
| siRNA 1204 | 1204 | GGCGATGATCCTAGTGACA | 2946 | TGTCACTAGGATCATCGCC |
| siRNA 1205 | 1205 | GCGATGATCCTAGTGACAA | 2947 | TTGTCACTAGGATCATCGC |
| siRNA 1206 | 1206 | CGATGATCCTAGTGACAAG | 2948 | CTTGTCACTAGGATCATCG |
| siRNA 1207 | 1207 | GATGATCCTAGTGACAAGT | 2949 | ACTTGTCACTAGGATCATC |
| siRNA 1208 | 1208 | ATGATCCTAGTGACAAGTT | 2950 | AACTTGTCACTAGGATCAT |
| siRNA 1209 | 1209 | TGATCCTAGTGACAAGTTT | 2951 | AAACTTGTCACTAGGATCA |
| siRNA 1210 | 1210 | GATCCTAGTGACAAGTTTT | 2952 | AAAACTTGTCACTAGGATC |
| siRNA 1211 | 1211 | ATCCTAGTGACAAGTTTTT | 2953 | AAAAACTTGTCACTAGGAT |
| siRNA 1212 | 1212 | TCCTAGTGACAAGTTTTTC | 2954 | GAAAAACTTGTCACTAGGA |
| siRNA 1213 | 1213 | CCTAGTGACAAGTTTTTCA | 2955 | TGAAAAACTTGTCACTAGG |
| siRNA 1214 | 1214 | CTAGTGACAAGTTTTTCAC | 2956 | GTGAAAAACTTGTCACTAG |
| siRNA 1215 | 1215 | TAGTGACAAGTTTTTCACA | 2957 | TGTGAAAAACTTGTCACTA |
| siRNA 1216 | 1216 | AGTGACAAGTTTTTCACAT | 2958 | ATGTGAAAAACTTGTCACT |
| siRNA 1217 | 1217 | GTGACAAGTTTTTCACATC | 2959 | GATGTGAAAAACTTGTCAC |
| siRNA 1218 | 1218 | TGACAAGTTTTTCACATCC | 2960 | GGATGTGAAAAACTTGTCA |
| siRNA 1219 | 1219 | GACAAGTTTTTCACATCCC | 2961 | GGGATGTGAAAAACTTGTC |
| siRNA 1220 | 1220 | ACAAGTTTTTCACATCCCA | 2962 | TGGGATGTGAAAAACTTGT |
| siRNA 1221 | 1221 | CAAGTTTTTCACATCCCAT | 2963 | ATGGGATGTGAAAAACTTG |
| siRNA 1222 | 1222 | AAGTTTTTCACATCCCATA | 2964 | TATGGGATGTGAAAAACTT |
| siRNA 1223 | 1223 | AGTTTTTCACATCCCATAA | 2965 | TTATGGGATGTGAAAAACT |
| siRNA 1224 | 1224 | GTTTTTCACATCCCATAAT | 2966 | ATTATGGGATGTGAAAAAC |
| siRNA 1225 | 1225 | TTTTTCACATCCCATAATG | 2967 | CATTATGGGATGTGAAAAA |
| siRNA 1226 | 1226 | TTTTCACATCCCATAATGG | 2968 | CCATTATGGGATGTGAAAA |
| siRNA 1227 | 1227 | TTTCACATCCCATAATGGC | 2969 | GCCATTATGGGATGTGAAA |
| siRNA 1228 | 1228 | TTCACATCCCATAATGGCA | 2970 | TGCCATTATGGGATGTGAA |
| siRNA 1229 | 1229 | TCACATCCCATAATGGCAT | 2971 | ATGCCATTATGGGATGTGA |
| siRNA 1230 | 1230 | CACATCCCATAATGGCATG | 2972 | CATGCCATTATGGGATGTG |
| siRNA 1231 | 1231 | ACATCCCATAATGGCATGC | 2973 | GCATGCCATTATGGGATGT |
| siRNA 1232 | 1232 | CATCCCATAATGGCATGCA | 2974 | TGCATGCCATTATGGGATG |
| siRNA 1233 | 1233 | ATCCCATAATGGCATGCAG | 2975 | CTGCATGCCATTATGGGAT |
| siRNA 1234 | 1234 | TCCCATAATGGCATGCAGT | 2976 | ACTGCATGCCATTATGGGA |
| siRNA 1235 | 1235 | CCCATAATGGCATGCAGTT | 2977 | AACTGCATGCCATTATGGG |
| siRNA 1236 | 1236 | CCATAATGGCATGCAGTTC | 2978 | GAACTGCATGCCATTATGG |
| siRNA 1237 | 1237 | CATAATGGCATGCAGTTCA | 2979 | TGAACTGCATGCCATTATG |
| siRNA 1238 | 1238 | ATAATGGCATGCAGTTCAG | 2980 | CTGAACTGCATGCCATTAT |
| siRNA 1239 | 1239 | TAATGGCATGCAGTTCAGT | 2981 | ACTGAACTGCATGCCATTA |
| siRNA 1240 | 1240 | AATGGCATGCAGTTCAGTA | 2982 | TACTGAACTGCATGCCATT |
| siRNA 1241 | 1241 | ATGGCATGCAGTTCAGTAC | 2983 | GTACTGAACTGCATGCCAT |
| siRNA 1242 | 1242 | TGGCATGCAGTTCAGTACC | 2984 | GGTACTGAACTGCATGCCA |
| siRNA 1243 | 1243 | GGCATGCAGTTCAGTACCT | 2985 | AGGTACTGAACTGCATGCC |
| siRNA 1244 | 1244 | GCATGCAGTTCAGTACCTG | 2986 | CAGGTACTGAACTGCATGC |
| siRNA 1245 | 1245 | CATGCAGTTCAGTACCTGG | 2987 | CCAGGTACTGAACTGCATG |
| siRNA 1246 | 1246 | ATGCAGTTCAGTACCTGGG | 2988 | CCCAGGTACTGAACTGCAT |
| siRNA 1247 | 1247 | TGCAGTTCAGTACCTGGGA | 2989 | TCCCAGGTACTGAACTGCA |
| siRNA 1248 | 1248 | GCAGTTCAGTACCTGGGAC | 2990 | GTCCCAGGTACTGAACTGC |
| siRNA 1249 | 1249 | CAGTTCAGTACCTGGGACA | 2991 | TGTCCCAGGTACTGAACTG |
| siRNA 1250 | 1250 | AGTTCAGTACCTGGGACAA | 2992 | TTGTCCCAGGTACTGAACT |
| siRNA 1251 | 1251 | GTTCAGTACCTGGGACAAT | 2993 | ATTGTCCCAGGTACTGAAC |
| siRNA 1252 | 1252 | TTCAGTACCTGGGACAATG | 2994 | CATTGTCCCAGGTACTGAA |
| siRNA 1253 | 1253 | TCAGTACCTGGGACAATGA | 2995 | TCATTGTCCCAGGTACTGA |
| siRNA 1254 | 1254 | CAGTACCTGGGACAATGAC | 2996 | GTCATTGTCCCAGGTACTG |
| siRNA 1255 | 1255 | AGTACCTGGGACAATGACA | 2997 | TGTCATTGTCCCAGGTACT |
| siRNA 1256 | 1256 | GTACCTGGGACAATGACAA | 2998 | TTGTCATTGTCCCAGGTAC |
| siRNA 1257 | 1257 | TACCTGGGACAATGACAAT | 2999 | ATTGTCATTGTCCCAGGTA |
| siRNA 1258 | 1258 | ACCTGGGACAATGACAATG | 3000 | CATTGTCATTGTCCCAGGT |
| siRNA 1259 | 1259 | CCTGGGACAATGACAATGA | 3001 | TCATTGTCATTGTCCCAGG |
| siRNA 1260 | 1260 | CTGGGACAATGACAATGAT | 3002 | ATCATTGTCATTGTCCCAG |
| siRNA 1261 | 1261 | TGGGACAATGACAATGATA | 3003 | TATCATTGTCATTGTCCCA |
| siRNA 1262 | 1262 | GGGACAATGACAATGATAA | 3004 | TTATCATTGTCATTGTCCC |
| siRNA 1263 | 1263 | GGACAATGACAATGATAAG | 3005 | CTTATCATTGTCATTGTCC |
| siRNA 1264 | 1264 | GACAATGACAATGATAAGT | 3006 | ACTTATCATTGTCATTGTC |
| siRNA 1265 | 1265 | ACAATGACAATGATAAGTT | 3007 | AACTTATCATTGTCATTGT |
| siRNA 1266 | 1266 | CAATGACAATGATAAGTTT | 3008 | AAACTTATCATTGTCATTG |
| siRNA 1267 | 1267 | AATGACAATGATAAGTTTG | 3009 | CAAACTTATCATTGTCATT |
| siRNA 1268 | 1268 | ATGACAATGATAAGTTTGA | 3010 | TCAAACTTATCATTGTCAT |
| siRNA 1269 | 1269 | TGACAATGATAAGTTTGAA | 3011 | TTCAAACTTATCATTGTCA |
| siRNA 1270 | 1270 | GACAATGATAAGTTTGAAG | 3012 | CTTCAAACTTATCATTGTC |
| siRNA 1271 | 1271 | ACAATGATAAGTTTGAAGG | 3013 | CCTTCAAACTTATCATTGT |
| siRNA 1272 | 1272 | CAATGATAAGTTTGAAGGC | 3014 | GCCTTCAAACTTATCATTG |
| siRNA 1273 | 1273 | AATGATAAGTTTGAAGGCA | 3015 | TGCCTTCAAACTTATCATT |
| siRNA 1274 | 1274 | ATGATAAGTTTGAAGGCAA | 3016 | TTGCCTTCAAACTTATCAT |
| siRNA 1275 | 1275 | TGATAAGTTTGAAGGCAAC | 3017 | GTTGCCTTCAAACTTATCA |
| siRNA 1276 | 1276 | GATAAGTTTGAAGGCAACT | 3018 | AGTTGCCTTCAAACTTATC |
| siRNA 1277 | 1277 | ATAAGTTTGAAGGCAACTG | 3019 | CAGTTGCCTTCAAACTTAT |
| siRNA 1278 | 1278 | TAAGTTTGAAGGCAACTGT | 3020 | ACAGTTGCCTTCAAACTTA |
| siRNA 1279 | 1279 | AAGTTTGAAGGCAACTGTG | 3021 | CACAGTTGCCTTCAAACTT |
| siRNA 1280 | 1280 | AGTTTGAAGGCAACTGTGC | 3022 | GCACAGTTGCCTTCAAACT |
| siRNA 1281 | 1281 | GTTTGAAGGCAACTGTGCT | 3023 | AGCACAGTTGCCTTCAAAC |
| siRNA 1282 | 1282 | TTTGAAGGCAACTGTGCTG | 3024 | CAGCACAGTTGCCTTCAAA |
| siRNA 1283 | 1283 | TTGAAGGCAACTGTGCTGA | 3025 | TCAGCACAGTTGCCTTCAA |
| siRNA 1284 | 1284 | TGAAGGCAACTGTGCTGAA | 3026 | TTCAGCACAGTTGCCTTCA |
| siRNA 1285 | 1285 | GAAGGCAACTGTGCTGAAC | 3027 | GTTCAGCACAGTTGCCTTC |
| siRNA 1286 | 1286 | AAGGCAACTGTGCTGAACA | 3028 | TGTTCAGCACAGTTGCCTT |
| siRNA 1287 | 1287 | AGGCAACTGTGCTGAACAG | 3029 | CTGTTCAGCACAGTTGCCT |
| siRNA 1288 | 1288 | GGCAACTGTGCTGAACAGG | 3030 | CCTGTTCAGCACAGTTGCC |
| siRNA 1289 | 1289 | GCAACTGTGCTGAACAGGA | 3031 | TCCTGTTCAGCACAGTTGC |
| siRNA 1290 | 1290 | CAACTGTGCTGAACAGGAT | 3032 | ATCCTGTTCAGCACAGTTG |
| siRNA 1291 | 1291 | AACTGTGCTGAACAGGATG | 3033 | CATCCTGTTCAGCACAGTT |
| siRNA 1292 | 1292 | ACTGTGCTGAACAGGATGG | 3034 | CCATCCTGTTCAGCACAGT |
| siRNA 1293 | 1293 | CTGTGCTGAACAGGATGGA | 3035 | TCCATCCTGTTCAGCACAG |
| siRNA 1294 | 1294 | TGTGCTGAACAGGATGGAT | 3036 | ATCCATCCTGTTCAGCACA |
| siRNA 1295 | 1295 | GTGCTGAACAGGATGGATC | 3037 | GATCCATCCTGTTCAGCAC |
| siRNA 1296 | 1296 | TGCTGAACAGGATGGATCT | 3038 | AGATCCATCCTGTTCAGCA |
| siRNA 1297 | 1297 | GCTGAACAGGATGGATCTG | 3039 | CAGATCCATCCTGTTCAGC |
| siRNA 1298 | 1298 | CTGAACAGGATGGATCTGG | 3040 | CCAGATCCATCCTGTTCAG |
| siRNA 1299 | 1299 | TGAACAGGATGGATCTGGT | 3041 | ACCAGATCCATCCTGTTCA |
| siRNA 1300 | 1300 | GAACAGGATGGATCTGGTT | 3042 | AACCAGATCCATCCTGTTC |
| siRNA 1301 | 1301 | AACAGGATGGATCTGGTTG | 3043 | CAACCAGATCCATCCTGTT |
| siRNA 1302 | 1302 | ACAGGATGGATCTGGTTGG | 3044 | CCAACCAGATCCATCCTGT |
| siRNA 1303 | 1303 | CAGGATGGATCTGGTTGGT | 3045 | ACCAACCAGATCCATCCTG |
| siRNA 1304 | 1304 | AGGATGGATCTGGTTGGTG | 3046 | CACCAACCAGATCCATCCT |
| siRNA 1305 | 1305 | GGATGGATCTGGTTGGTGG | 3047 | CCACCAACCAGATCCATCC |
| siRNA 1306 | 1306 | GATGGATCTGGTTGGTGGA | 3048 | TCCACCAACCAGATCCATC |
| siRNA 1307 | 1307 | ATGGATCTGGTTGGTGGAT | 3049 | ATCCACCAACCAGATCCAT |
| siRNA 1308 | 1308 | TGGATCTGGTTGGTGGATG | 3050 | CATCCACCAACCAGATCCA |
| siRNA 1309 | 1309 | GGATCTGGTTGGTGGATGA | 3051 | TCATCCACCAACCAGATCC |
| siRNA 1310 | 1310 | GATCTGGTTGGTGGATGAA | 3052 | TTCATCCACCAACCAGATC |
| siRNA 1311 | 1311 | ATCTGGTTGGTGGATGAAC | 3053 | GTTCATCCACCAACCAGAT |
| siRNA 1312 | 1312 | TCTGGTTGGTGGATGAACA | 3054 | TGTTCATCCACCAACCAGA |
| siRNA 1313 | 1313 | CTGGTTGGTGGATGAACAA | 3055 | TTGTTCATCCACCAACCAG |
| siRNA 1314 | 1314 | TGGTTGGTGGATGAACAAG | 3056 | CTTGTTCATCCACCAACCA |
| siRNA 1315 | 1315 | GGTTGGTGGATGAACAAGT | 3057 | ACTTGTTCATCCACCAACC |
| siRNA 1316 | 1316 | GTTGGTGGATGAACAAGTG | 3058 | CACTTGTTCATCCACCAAC |
| siRNA 1317 | 1317 | TTGGTGGATGAACAAGTGT | 3059 | ACACTTGTTCATCCACCAA |
| siRNA 1318 | 1318 | TGGTGGATGAACAAGTGTC | 3060 | GACACTTGTTCATCCACCA |
| siRNA 1319 | 1319 | GGTGGATGAACAAGTGTCA | 3061 | TGACACTTGTTCATCCACC |
| siRNA 1320 | 1320 | GTGGATGAACAAGTGTCAC | 3062 | GTGACACTTGTTCATCCAC |
| siRNA 1321 | 1321 | TGGATGAACAAGTGTCACG | 3063 | CGTGACACTTGTTCATCCA |
| siRNA 1322 | 1322 | GGATGAACAAGTGTCACGC | 3064 | GCGTGACACTTGTTCATCC |
| siRNA 1323 | 1323 | GATGAACAAGTGTCACGCT | 3065 | AGCGTGACACTTGTTCATC |
| siRNA 1324 | 1324 | ATGAACAAGTGTCACGCTG | 3066 | CAGCGTGACACTTGTTCAT |
| siRNA 1325 | 1325 | TGAACAAGTGTCACGCTGG | 3067 | CCAGCGTGACACTTGTTCA |
| siRNA 1326 | 1326 | GAACAAGTGTCACGCTGGC | 3068 | GCCAGCGTGACACTTGTTC |
| siRNA 1327 | 1327 | AACAAGTGTCACGCTGGCC | 3069 | GGCCAGCGTGACACTTGTT |
| siRNA 1328 | 1328 | ACAAGTGTCACGCTGGCCA | 3070 | TGGCCAGCGTGACACTTGT |
| siRNA 1329 | 1329 | CAAGTGTCACGCTGGCCAT | 3071 | ATGGCCAGCGTGACACTTG |
| siRNA 1330 | 1330 | AAGTGTCACGCTGGCCATC | 3072 | GATGGCCAGCGTGACACTT |
| siRNA 1331 | 1331 | AGTGTCACGCTGGCCATCT | 3073 | AGATGGCCAGCGTGACACT |
| siRNA 1332 | 1332 | GTGTCACGCTGGCCATCTC | 3074 | GAGATGGCCAGCGTGACAC |
| siRNA 1333 | 1333 | TGTCACGCTGGCCATCTCA | 3075 | TGAGATGGCCAGCGTGACA |
| siRNA 1334 | 1334 | GTCACGCTGGCCATCTCAA | 3076 | TTGAGATGGCCAGCGTGAC |
| siRNA 1335 | 1335 | TCACGCTGGCCATCTCAAT | 3077 | ATTGAGATGGCCAGCGTGA |
| siRNA 1336 | 1336 | CACGCTGGCCATCTCAATG | 3078 | CATTGAGATGGCCAGCGTG |
| siRNA 1337 | 1337 | ACGCTGGCCATCTCAATGG | 3079 | CCATTGAGATGGCCAGCGT |
| siRNA 1338 | 1338 | CGCTGGCCATCTCAATGGA | 3080 | TCCATTGAGATGGCCAGCG |
| siRNA 1339 | 1339 | GCTGGCCATCTCAATGGAG | 3081 | CTCCATTGAGATGGCCAGC |
| siRNA 1340 | 1340 | CTGGCCATCTCAATGGAGT | 3082 | ACTCCATTGAGATGGCCAG |
| siRNA 1341 | 1341 | TGGCCATCTCAATGGAGTT | 3083 | AACTCCATTGAGATGGCCA |
| siRNA 1342 | 1342 | GGCCATCTCAATGGAGTTT | 3084 | AAACTCCATTGAGATGGCC |
| siRNA 1343 | 1343 | GCCATCTCAATGGAGTTTA | 3085 | TAAACTCCATTGAGATGGC |
| siRNA 1344 | 1344 | CCATCTCAATGGAGTTTAT | 3086 | ATAAACTCCATTGAGATGG |
| siRNA 1345 | 1345 | CATCTCAATGGAGTTTATT | 3087 | AATAAACTCCATTGAGATG |
| siRNA 1346 | 1346 | ATCTCAATGGAGTTTATTA | 3088 | TAATAAACTCCATTGAGAT |
| siRNA 1347 | 1347 | TCTCAATGGAGTTTATTAC | 3089 | GTAATAAACTCCATTGAGA |
| siRNA 1348 | 1348 | CTCAATGGAGTTTATTACC | 3090 | GGTAATAAACTCCATTGAG |
| siRNA 1349 | 1349 | TCAATGGAGTTTATTACCA | 3091 | TGGTAATAAACTCCATTGA |
| siRNA 1350 | 1350 | CAATGGAGTTTATTACCAA | 3092 | TTGGTAATAAACTCCATTG |
| siRNA 1351 | 1351 | AATGGAGTTTATTACCAAG | 3093 | CTTGGTAATAAACTCCATT |
| siRNA 1352 | 1352 | ATGGAGTTTATTACCAAGG | 3094 | CCTTGGTAATAAACTCCAT |
| siRNA 1353 | 1353 | TGGAGTTTATTACCAAGGT | 3095 | ACCTTGGTAATAAACTCCA |
| siRNA 1354 | 1354 | GGAGTTTATTACCAAGGTG | 3096 | CACCTTGGTAATAAACTCC |
| siRNA 1355 | 1355 | GAGTTTATTACCAAGGTGG | 3097 | CCACCTTGGTAATAAACTC |
| siRNA 1356 | 1356 | AGTTTATTACCAAGGTGGC | 3098 | GCCACCTTGGTAATAAACT |
| siRNA 1357 | 1357 | GTTTATTACCAAGGTGGCA | 3099 | TGCCACCTTGGTAATAAAC |
| siRNA 1358 | 1358 | TTTATTACCAAGGTGGCAC | 3100 | GTGCCACCTTGGTAATAAA |
| siRNA 1359 | 1359 | TTATTACCAAGGTGGCACT | 3101 | AGTGCCACCTTGGTAATAA |
| siRNA 1360 | 1360 | TATTACCAAGGTGGCACTT | 3102 | AAGTGCCACCTTGGTAATA |
| siRNA 1361 | 1361 | ATTACCAAGGTGGCACTTA | 3103 | TAAGTGCCACCTTGGTAAT |
| siRNA 1362 | 1362 | TTACCAAGGTGGCACTTAC | 3104 | GTAAGTGCCACCTTGGTAA |
| siRNA 1363 | 1363 | TACCAAGGTGGCACTTACT | 3105 | AGTAAGTGCCACCTTGGTA |
| siRNA 1364 | 1364 | ACCAAGGTGGCACTTACTC | 3106 | GAGTAAGTGCCACCTTGGT |
| siRNA 1365 | 1365 | CCAAGGTGGCACTTACTCA | 3107 | TGAGTAAGTGCCACCTTGG |
| siRNA 1366 | 1366 | CAAGGTGGCACTTACTCAA | 3108 | TTGAGTAAGTGCCACCTTG |
| siRNA 1367 | 1367 | AAGGTGGCACTTACTCAAA | 3109 | TTTGAGTAAGTGCCACCTT |
| siRNA 1368 | 1368 | AGGTGGCACTTACTCAAAA | 3110 | TTTTGAGTAAGTGCCACCT |
| siRNA 1369 | 1369 | GGTGGCACTTACTCAAAAG | 3111 | CTTTTGAGTAAGTGCCACC |
| siRNA 1370 | 1370 | GTGGCACTTACTCAAAAGC | 3112 | GCTTTTGAGTAAGTGCCAC |
| siRNA 1371 | 1371 | TGGCACTTACTCAAAAGCA | 3113 | TGCTTTTGAGTAAGTGCCA |
| siRNA 1372 | 1372 | GGCACTTACTCAAAAGCAT | 3114 | ATGCTTTTGAGTAAGTGCC |
| siRNA 1373 | 1373 | GCACTTACTCAAAAGCATC | 3115 | GATGCTTTTGAGTAAGTGC |
| siRNA 1374 | 1374 | CACTTACTCAAAAGCATCT | 3116 | AGATGCTTTTGAGTAAGTG |
| siRNA 1375 | 1375 | ACTTACTCAAAAGCATCTA | 3117 | TAGATGCTTTTGAGTAAGT |
| siRNA 1376 | 1376 | CTTACTCAAAAGCATCTAC | 3118 | GTAGATGCTTTTGAGTAAG |
| siRNA 1377 | 1377 | TTACTCAAAAGCATCTACT | 3119 | AGTAGATGCTTTTGAGTAA |
| siRNA 1378 | 1378 | TACTCAAAAGCATCTACTC | 3120 | GAGTAGATGCTTTTGAGTA |
| siRNA 1379 | 1379 | ACTCAAAAGCATCTACTCC | 3121 | GGAGTAGATGCTTTTGAGT |
| siRNA 1380 | 1380 | CTCAAAAGCATCTACTCCT | 3122 | AGGAGTAGATGCTTTTGAG |
| siRNA 1381 | 1381 | TCAAAAGCATCTACTCCTA | 3123 | TAGGAGTAGATGCTTTTGA |
| siRNA 1382 | 1382 | CAAAAGCATCTACTCCTAA | 3124 | TTAGGAGTAGATGCTTTTG |
| siRNA 1383 | 1383 | AAAAGCATCTACTCCTAAT | 3125 | ATTAGGAGTAGATGCTTTT |
| siRNA 1384 | 1384 | AAAGCATCTACTCCTAATG | 3126 | CATTAGGAGTAGATGCTTT |
| siRNA 1385 | 1385 | AAGCATCTACTCCTAATGG | 3127 | CCATTAGGAGTAGATGCTT |
| siRNA 1386 | 1386 | AGCATCTACTCCTAATGGT | 3128 | ACCATTAGGAGTAGATGCT |
| siRNA 1387 | 1387 | GCATCTACTCCTAATGGTT | 3129 | AACCATTAGGAGTAGATGC |
| siRNA 1388 | 1388 | CATCTACTCCTAATGGTTA | 3130 | TAACCATTAGGAGTAGATG |
| siRNA 1389 | 1389 | ATCTACTCCTAATGGTTAT | 3131 | ATAACCATTAGGAGTAGAT |
| siRNA 1390 | 1390 | TCTACTCCTAATGGTTATG | 3132 | CATAACCATTAGGAGTAGA |
| siRNA 1391 | 1391 | CTACTCCTAATGGTTATGA | 3133 | TCATAACCATTAGGAGTAG |
| siRNA 1392 | 1392 | TACTCCTAATGGTTATGAT | 3134 | ATCATAACCATTAGGAGTA |
| siRNA 1393 | 1393 | ACTCCTAATGGTTATGATA | 3135 | TATCATAACCATTAGGAGT |
| siRNA 1394 | 1394 | CTCCTAATGGTTATGATAA | 3136 | TTATCATAACCATTAGGAG |
| siRNA 1395 | 1395 | TCCTAATGGTTATGATAAT | 3137 | ATTATCATAACCATTAGGA |
| siRNA 1396 | 1396 | CCTAATGGTTATGATAATG | 3138 | CATTATCATAACCATTAGG |
| siRNA 1397 | 1397 | CTAATGGTTATGATAATGG | 3139 | CCATTATCATAACCATTAG |
| siRNA 1398 | 1398 | TAATGGTTATGATAATGGC | 3140 | GCCATTATCATAACCATTA |
| siRNA 1399 | 1399 | AATGGTTATGATAATGGCA | 3141 | TGCCATTATCATAACCATT |
| siRNA 1400 | 1400 | ATGGTTATGATAATGGCAT | 3142 | ATGCCATTATCATAACCAT |
| siRNA 1401 | 1401 | TGGTTATGATAATGGCATT | 3143 | AATGCCATTATCATAACCA |
| siRNA 1402 | 1402 | GGTTATGATAATGGCATTA | 3144 | TAATGCCATTATCATAACC |
| siRNA 1403 | 1403 | GTTATGATAATGGCATTAT | 3145 | ATAATGCCATTATCATAAC |
| siRNA 1404 | 1404 | TTATGATAATGGCATTATT | 3146 | AATAATGCCATTATCATAA |
| siRNA 1405 | 1405 | TATGATAATGGCATTATTT | 3147 | AAATAATGCCATTATCATA |
| siRNA 1406 | 1406 | ATGATAATGGCATTATTTG | 3148 | CAAATAATGCCATTATCAT |
| siRNA 1407 | 1407 | TGATAATGGCATTATTTGG | 3149 | CCAAATAATGCCATTATCA |
| siRNA 1408 | 1408 | GATAATGGCATTATTTGGG | 3150 | CCCAAATAATGCCATTATC |
| siRNA 1409 | 1409 | ATAATGGCATTATTTGGGC | 3151 | GCCCAAATAATGCCATTAT |
| siRNA 1410 | 1410 | TAATGGCATTATTTGGGCC | 3152 | GGCCCAAATAATGCCATTA |
| siRNA 1411 | 1411 | AATGGCATTATTTGGGCCA | 3153 | TGGCCCAAATAATGCCATT |
| siRNA 1412 | 1412 | ATGGCATTATTTGGGCCAC | 3154 | GTGGCCCAAATAATGCCAT |
| siRNA 1413 | 1413 | TGGCATTATTTGGGCCACT | 3155 | AGTGGCCCAAATAATGCCA |
| siRNA 1414 | 1414 | GGCATTATTTGGGCCACTT | 3156 | AAGTGGCCCAAATAATGCC |
| siRNA 1415 | 1415 | GCATTATTTGGGCCACTTG | 3157 | CAAGTGGCCCAAATAATGC |
| siRNA 1416 | 1416 | CATTATTTGGGCCACTTGG | 3158 | CCAAGTGGCCCAAATAATG |
| siRNA 1417 | 1417 | ATTATTTGGGCCACTTGGA | 3159 | TCCAAGTGGCCCAAATAAT |
| siRNA 1418 | 1418 | TTATTTGGGCCACTTGGAA | 3160 | TTCCAAGTGGCCCAAATAA |
| siRNA 1419 | 1419 | TATTTGGGCCACTTGGAAA | 3161 | TTTCCAAGTGGCCCAAATA |
| siRNA 1420 | 1420 | ATTTGGGCCACTTGGAAAA | 3162 | TTTTCCAAGTGGCCCAAAT |
| siRNA 1421 | 1421 | TTTGGGCCACTTGGAAAAC | 3163 | GTTTTCCAAGTGGCCCAAA |
| siRNA 1422 | 1422 | TTGGGCCACTTGGAAAACC | 3164 | GGTTTTCCAAGTGGCCCAA |
| siRNA 1423 | 1423 | TGGGCCACTTGGAAAACCC | 3165 | GGGTTTTCCAAGTGGCCCA |
| siRNA 1424 | 1424 | GGGCCACTTGGAAAACCCG | 3166 | CGGGTTTTCCAAGTGGCCC |
| siRNA 1425 | 1425 | GGCCACTTGGAAAACCCGG | 3167 | CCGGGTTTTCCAAGTGGCC |
| siRNA 1426 | 1426 | GCCACTTGGAAAACCCGGT | 3168 | ACCGGGTTTTCCAAGTGGC |
| siRNA 1427 | 1427 | CCACTTGGAAAACCCGGTG | 3169 | CACCGGGTTTTCCAAGTGG |
| siRNA 1428 | 1428 | CACTTGGAAAACCCGGTGG | 3170 | CCACCGGGTTTTCCAAGTG |
| siRNA 1429 | 1429 | ACTTGGAAAACCCGGTGGT | 3171 | ACCACCGGGTTTTCCAAGT |
| siRNA 1430 | 1430 | CTTGGAAAACCCGGTGGTA | 3172 | TACCACCGGGTTTTCCAAG |
| siRNA 1431 | 1431 | TTGGAAAACCCGGTGGTAT | 3173 | ATACCACCGGGTTTTCCAA |
| siRNA 1432 | 1432 | TGGAAAACCCGGTGGTATT | 3174 | AATACCACCGGGTTTTCCA |
| siRNA 1433 | 1433 | GGAAAACCCGGTGGTATTC | 3175 | GAATACCACCGGGTTTTCC |
| siRNA 1434 | 1434 | GAAAACCCGGTGGTATTCC | 3176 | GGAATACCACCGGGTTTTC |
| siRNA 1435 | 1435 | AAAACCCGGTGGTATTCCA | 3177 | TGGAATACCACCGGGTTTT |
| siRNA 1436 | 1436 | AAACCCGGTGGTATTCCAT | 3178 | ATGGAATACCACCGGGTTT |
| siRNA 1437 | 1437 | AACCCGGTGGTATTCCATG | 3179 | CATGGAATACCACCGGGTT |
| siRNA 1438 | 1438 | ACCCGGTGGTATTCCATGA | 3180 | TCATGGAATACCACCGGGT |
| siRNA 1439 | 1439 | CCCGGTGGTATTCCATGAA | 3181 | TTCATGGAATACCACCGGG |
| siRNA 1440 | 1440 | CCGGTGGTATTCCATGAAG | 3182 | CTTCATGGAATACCACCGG |
| siRNA 1441 | 1441 | CGGTGGTATTCCATGAAGA | 3183 | TCTTCATGGAATACCACCG |
| siRNA 1442 | 1442 | GGTGGTATTCCATGAAGAA | 3184 | TTCTTCATGGAATACCACC |
| siRNA 1443 | 1443 | GTGGTATTCCATGAAGAAA | 3185 | TTTCTTCATGGAATACCAC |
| siRNA 1444 | 1444 | TGGTATTCCATGAAGAAAA | 3186 | TTTTCTTCATGGAATACCA |
| siRNA 1445 | 1445 | GGTATTCCATGAAGAAAAC | 3187 | GTTTTCTTCATGGAATACC |
| siRNA 1446 | 1446 | GTATTCCATGAAGAAAACC | 3188 | GGTTTTCTTCATGGAATAC |
| siRNA 1447 | 1447 | TATTCCATGAAGAAAACCA | 3189 | TGGTTTTCTTCATGGAATA |
| siRNA 1448 | 1448 | ATTCCATGAAGAAAACCAC | 3190 | GTGGTTTTCTTCATGGAAT |
| siRNA 1449 | 1449 | TTCCATGAAGAAAACCACT | 3191 | AGTGGTTTTCTTCATGGAA |
| siRNA 1450 | 1450 | TCCATGAAGAAAACCACTA | 3192 | TAGTGGTTTTCTTCATGGA |
| siRNA 1451 | 1451 | CCATGAAGAAAACCACTAT | 3193 | ATAGTGGTTTTCTTCATGG |
| siRNA 1452 | 1452 | CATGAAGAAAACCACTATG | 3194 | CATAGTGGTTTTCTTCATG |
| siRNA 1453 | 1453 | ATGAAGAAAACCACTATGA | 3195 | TCATAGTGGTTTTCTTCAT |
| siRNA 1454 | 1454 | TGAAGAAAACCACTATGAA | 3196 | TTCATAGTGGTTTTCTTCA |
| siRNA 1455 | 1455 | GAAGAAAACCACTATGAAG | 3197 | CTTCATAGTGGTTTTCTTC |
| siRNA 1456 | 1456 | AAGAAAACCACTATGAAGA | 3198 | TCTTCATAGTGGTTTTCTT |
| siRNA 1457 | 1457 | AGAAAACCACTATGAAGAT | 3199 | ATCTTCATAGTGGTTTTCT |
| siRNA 1458 | 1458 | GAAAACCACTATGAAGATA | 3200 | TATCTTCATAGTGGTTTTC |
| siRNA 1459 | 1459 | AAAACCACTATGAAGATAA | 3201 | TTATCTTCATAGTGGTTTT |
| siRNA 1460 | 1460 | AAACCACTATGAAGATAAT | 3202 | ATTATCTTCATAGTGGTTT |
| siRNA 1461 | 1461 | AACCACTATGAAGATAATC | 3203 | GATTATCTTCATAGTGGTT |
| siRNA 1462 | 1462 | ACCACTATGAAGATAATCC | 3204 | GGATTATCTTCATAGTGGT |
| siRNA 1463 | 1463 | CCACTATGAAGATAATCCC | 3205 | GGGATTATCTTCATAGTGG |
| siRNA 1464 | 1464 | CACTATGAAGATAATCCCA | 3206 | TGGGATTATCTTCATAGTG |
| siRNA 1465 | 1465 | ACTATGAAGATAATCCCAT | 3207 | ATGGGATTATCTTCATAGT |
| siRNA 1466 | 1466 | CTATGAAGATAATCCCATT | 3208 | AATGGGATTATCTTCATAG |
| siRNA 1467 | 1467 | TATGAAGATAATCCCATTC | 3209 | GAATGGGATTATCTTCATA |
| siRNA 1468 | 1468 | ATGAAGATAATCCCATTCA | 3210 | TGAATGGGATTATCTTCAT |
| siRNA 1469 | 1469 | TGAAGATAATCCCATTCAA | 3211 | TTGAATGGGATTATCTTCA |
| siRNA 1470 | 1470 | GAAGATAATCCCATTCAAC | 3212 | GTTGAATGGGATTATCTTC |
| siRNA 1471 | 1471 | AAGATAATCCCATTCAACA | 3213 | TGTTGAATGGGATTATCTT |
| siRNA 1472 | 1472 | AGATAATCCCATTCAACAG | 3214 | CTGTTGAATGGGATTATCT |
| siRNA 1473 | 1473 | GATAATCCCATTCAACAGA | 3215 | TCTGTTGAATGGGATTATC |
| siRNA 1474 | 1474 | ATAATCCCATTCAACAGAC | 3216 | GTCTGTTGAATGGGATTAT |
| siRNA 1475 | 1475 | TAATCCCATTCAACAGACT | 3217 | AGTCTGTTGAATGGGATTA |
| siRNA 1476 | 1476 | AATCCCATTCAACAGACTC | 3218 | GAGTCTGTTGAATGGGATT |
| siRNA 1477 | 1477 | ATCCCATTCAACAGACTCA | 3219 | TGAGTCTGTTGAATGGGAT |
| siRNA 1478 | 1478 | TCCCATTCAACAGACTCAC | 3220 | GTGAGTCTGTTGAATGGGA |
| siRNA 1479 | 1479 | CCCATTCAACAGACTCACA | 3221 | TGTGAGTCTGTTGAATGGG |
| siRNA 1480 | 1480 | CCATTCAACAGACTCACAA | 3222 | TTGTGAGTCTGTTGAATGG |
| siRNA 1481 | 1481 | CATTCAACAGACTCACAAT | 3223 | ATTGTGAGTCTGTTGAATG |
| siRNA 1482 | 1482 | ATTCAACAGACTCACAATT | 3224 | AATTGTGAGTCTGTTGAAT |
| siRNA 1483 | 1483 | TTCAACAGACTCACAATTG | 3225 | CAATTGTGAGTCTGTTGAA |
| siRNA 1484 | 1484 | TCAACAGACTCACAATTGG | 3226 | CCAATTGTGAGTCTGTTGA |
| siRNA 1485 | 1485 | CAACAGACTCACAATTGGA | 3227 | TCCAATTGTGAGTCTGTTG |
| siRNA 1486 | 1486 | AACAGACTCACAATTGGAG | 3228 | CTCCAATTGTGAGTCTGTT |
| siRNA 1487 | 1487 | ACAGACTCACAATTGGAGA | 3229 | TCTCCAATTGTGAGTCTGT |
| siRNA 1488 | 1488 | CAGACTCACAATTGGAGAA | 3230 | TTCTCCAATTGTGAGTCTG |
| siRNA 1489 | 1489 | AGACTCACAATTGGAGAAG | 3231 | CTTCTCCAATTGTGAGTCT |
| siRNA 1490 | 1490 | GACTCACAATTGGAGAAGG | 3232 | CCTTCTCCAATTGTGAGTC |
| siRNA 1491 | 1491 | ACTCACAATTGGAGAAGGA | 3233 | TCCTTCTCCAATTGTGAGT |
| siRNA 1492 | 1492 | CTCACAATTGGAGAAGGAC | 3234 | GTCCTTCTCCAATTGTGAG |
| siRNA 1493 | 1493 | TCACAATTGGAGAAGGACA | 3235 | TGTCCTTCTCCAATTGTGA |
| siRNA 1494 | 1494 | CACAATTGGAGAAGGACAG | 3236 | CTGTCCTTCTCCAATTGTG |
| siRNA 1495 | 1495 | ACAATTGGAGAAGGACAGC | 3237 | GCTGTCCTTCTCCAATTGT |
| siRNA 1496 | 1496 | CAATTGGAGAAGGACAGCA | 3238 | TGCTGTCCTTCTCCAATTG |
| siRNA 1497 | 1497 | AATTGGAGAAGGACAGCAA | 3239 | TTGCTGTCCTTCTCCAATT |
| siRNA 1498 | 1498 | ATTGGAGAAGGACAGCAAC | 3240 | GTTGCTGTCCTTCTCCAAT |
| siRNA 1499 | 1499 | TTGGAGAAGGACAGCAACA | 3241 | TGTTGCTGTCCTTCTCCAA |
| siRNA 1500 | 1500 | TGGAGAAGGACAGCAACAC | 3242 | GTGTTGCTGTCCTTCTCCA |
| siRNA 1501 | 1501 | GGAGAAGGACAGCAACACC | 3243 | GGTGTTGCTGTCCTTCTCC |
| siRNA 1502 | 1502 | GAGAAGGACAGCAACACCA | 3244 | TGGTGTTGCTGTCCTTCTC |
| siRNA 1503 | 1503 | AGAAGGACAGCAACACCAC | 3245 | GTGGTGTTGCTGTCCTTCT |
| siRNA 1504 | 1504 | GAAGGACAGCAACACCACC | 3246 | GGTGGTGTTGCTGTCCTTC |
| siRNA 1505 | 1505 | AAGGACAGCAACACCACCT | 3247 | AGGTGGTGTTGCTGTCCTT |
| siRNA 1506 | 1506 | AGGACAGCAACACCACCTG | 3248 | CAGGTGGTGTTGCTGTCCT |
| siRNA 1507 | 1507 | GGACAGCAACACCACCTGG | 3249 | CCAGGTGGTGTTGCTGTCC |
| siRNA 1508 | 1508 | GACAGCAACACCACCTGGG | 3250 | CCCAGGTGGTGTTGCTGTC |
| siRNA 1509 | 1509 | ACAGCAACACCACCTGGGG | 3251 | CCCCAGGTGGTGTTGCTGT |
| siRNA 1510 | 1510 | CAGCAACACCACCTGGGGG | 3252 | CCCCCAGGTGGTGTTGCTG |
| siRNA 1511 | 1511 | AGCAACACCACCTGGGGGG | 3253 | CCCCCCAGGTGGTGTTGCT |
| siRNA 1512 | 1512 | GCAACACCACCTGGGGGGA | 3254 | TCCCCCCAGGTGGTGTTGC |
| siRNA 1513 | 1513 | CAACACCACCTGGGGGGAG | 3255 | CTCCCCCCAGGTGGTGTTG |
| siRNA 1514 | 1514 | AACACCACCTGGGGGGAGC | 3256 | GCTCCCCCCAGGTGGTGTT |
| siRNA 1515 | 1515 | ACACCACCTGGGGGGAGCC | 3257 | GGCTCCCCCCAGGTGGTGT |
| siRNA 1516 | 1516 | CACCACCTGGGGGGAGCCA | 3258 | TGGCTCCCCCCAGGTGGTG |
| siRNA 1517 | 1517 | ACCACCTGGGGGGAGCCAA | 3259 | TTGGCTCCCCCCAGGTGGT |
| siRNA 1518 | 1518 | CCACCTGGGGGGAGCCAAA | 3260 | TTTGGCTCCCCCCAGGTGG |
| siRNA 1519 | 1519 | CACCTGGGGGGAGCCAAAC | 3261 | GTTTGGCTCCCCCCAGGTG |
| siRNA 1520 | 1520 | ACCTGGGGGGAGCCAAACA | 3262 | TGTTTGGCTCCCCCCAGGT |
| siRNA 1521 | 1521 | CCTGGGGGGAGCCAAACAG | 3263 | CTGTTTGGCTCCCCCCAGG |
| siRNA 1522 | 1522 | CTGGGGGGAGCCAAACAGG | 3264 | CCTGTTTGGCTCCCCCCAG |
| siRNA 1523 | 1523 | TGGGGGGAGCCAAACAGGC | 3265 | GCCTGTTTGGCTCCCCCCA |
| siRNA 1524 | 1524 | GGGGGGAGCCAAACAGGCT | 3266 | AGCCTGTTTGGCTCCCCCC |
| siRNA 1525 | 1525 | GGGGGAGCCAAACAGGCTG | 3267 | CAGCCTGTTTGGCTCCCCC |
| siRNA 1526 | 1526 | GGGGAGCCAAACAGGCTGG | 3268 | CCAGCCTGTTTGGCTCCCC |
| siRNA 1527 | 1527 | GGGAGCCAAACAGGCTGGA | 3269 | TCCAGCCTGTTTGGCTCCC |
| siRNA 1528 | 1528 | GGAGCCAAACAGGCTGGAG | 3270 | CTCCAGCCTGTTTGGCTCC |
| siRNA 1529 | 1529 | GAGCCAAACAGGCTGGAGA | 3271 | TCTCCAGCCTGTTTGGCTC |
| siRNA 1530 | 1530 | AGCCAAACAGGCTGGAGAC | 3272 | GTCTCCAGCCTGTTTGGCT |
| siRNA 1531 | 1531 | GCCAAACAGGCTGGAGACG | 3273 | CGTCTCCAGCCTGTTTGGC |
| siRNA 1532 | 1532 | CCAAACAGGCTGGAGACGT | 3274 | ACGTCTCCAGCCTGTTTGG |
| siRNA 1533 | 1533 | CAAACAGGCTGGAGACGTT | 3275 | AACGTCTCCAGCCTGTTTG |
| siRNA 1534 | 1534 | AAACAGGCTGGAGACGTTT | 3276 | AAACGTCTCCAGCCTGTTT |
| siRNA 1535 | 1535 | AACAGGCTGGAGACGTTTA | 3277 | TAAACGTCTCCAGCCTGTT |
| siRNA 1536 | 1536 | ACAGGCTGGAGACGTTTAA | 3278 | TTAAACGTCTCCAGCCTGT |
| siRNA 1537 | 1537 | CAGGCTGGAGACGTTTAAA | 3279 | TTTAAACGTCTCCAGCCTG |
| siRNA 1538 | 1538 | AGGCTGGAGACGTTTAAAA | 3280 | TTTTAAACGTCTCCAGCCT |
| siRNA 1539 | 1539 | GGCTGGAGACGTTTAAAAG | 3281 | CTTTTAAACGTCTCCAGCC |
| siRNA 1540 | 1540 | GCTGGAGACGTTTAAAAGA | 3282 | TCTTTTAAACGTCTCCAGC |
| siRNA 1541 | 1541 | CTGGAGACGTTTAAAAGAC | 3283 | GTCTTTTAAACGTCTCCAG |
| siRNA 1542 | 1542 | TGGAGACGTTTAAAAGACC | 3284 | GGTCTTTTAAACGTCTCCA |
| siRNA 1543 | 1543 | GGAGACGTTTAAAAGACCG | 3285 | CGGTCTTTTAAACGTCTCC |
| siRNA 1544 | 1544 | GAGACGTTTAAAAGACCGT | 3286 | ACGGTCTTTTAAACGTCTC |
| siRNA 1545 | 1545 | AGACGTTTAAAAGACCGTT | 3287 | AACGGTCTTTTAAACGTCT |
| siRNA 1546 | 1546 | GACGTTTAAAAGACCGTTT | 3288 | AAACGGTCTTTTAAACGTC |
| siRNA 1547 | 1547 | ACGTTTAAAAGACCGTTTC | 3289 | GAAACGGTCTTTTAAACGT |
| siRNA 1548 | 1548 | CGTTTAAAAGACCGTTTCA | 3290 | TGAAACGGTCTTTTAAACG |
| siRNA 1549 | 1549 | GTTTAAAAGACCGTTTCAA | 3291 | TTGAAACGGTCTTTTAAAC |
| siRNA 1550 | 1550 | TTTAAAAGACCGTTTCAAA | 3292 | TTTGAAACGGTCTTTTAAA |
| siRNA 1551 | 1551 | TTAAAAGACCGTTTCAAAA | 3293 | TTTTGAAACGGTCTTTTAA |
| siRNA 1552 | 1552 | TAAAAGACCGTTTCAAAAG | 3294 | CTTTTGAAACGGTCTTTTA |
| siRNA 1553 | 1553 | AAAAGACCGTTTCAAAAGA | 3295 | TCTTTTGAAACGGTCTTTT |
| siRNA 1554 | 1554 | AAAGACCGTTTCAAAAGAG | 3296 | CTCTTTTGAAACGGTCTTT |
| siRNA 1555 | 1555 | AAGACCGTTTCAAAAGAGA | 3297 | TCTCTTTTGAAACGGTCTT |
| siRNA 1556 | 1556 | AGACCGTTTCAAAAGAGAT | 3298 | ATCTCTTTTGAAACGGTCT |
| siRNA 1557 | 1557 | GACCGTTTCAAAAGAGATT | 3299 | AATCTCTTTTGAAACGGTC |
| siRNA 1558 | 1558 | ACCGTTTCAAAAGAGATTT | 3300 | AAATCTCTTTTGAAACGGT |
| siRNA 1559 | 1559 | CCGTTTCAAAAGAGATTTA | 3301 | TAAATCTCTTTTGAAACGG |
| siRNA 1560 | 1560 | CGTTTCAAAAGAGATTTAC | 3302 | GTAAATCTCTTTTGAAACG |
| siRNA 1561 | 1561 | GTTTCAAAAGAGATTTACT | 3303 | AGTAAATCTCTTTTGAAAC |
| siRNA 1562 | 1562 | TTTCAAAAGAGATTTACTT | 3304 | AAGTAAATCTCTTTTGAAA |
| siRNA 1563 | 1563 | TTCAAAAGAGATTTACTTT | 3305 | AAAGTAAATCTCTTTTGAA |
| siRNA 1564 | 1564 | TCAAAAGAGATTTACTTTT | 3306 | AAAAGTAAATCTCTTTTGA |
| siRNA 1565 | 1565 | CAAAAGAGATTTACTTTTT | 3307 | AAAAAGTAAATCTCTTTTG |
| siRNA 1566 | 1566 | AAAAGAGATTTACTTTTTT | 3308 | AAAAAAGTAAATCTCTTTT |
| siRNA 1567 | 1567 | AAAGAGATTTACTTTTTTA | 3309 | TAAAAAAGTAAATCTCTTT |
| siRNA 1568 | 1568 | AAGAGATTTACTTTTTTAA | 3310 | TTAAAAAAGTAAATCTCTT |
| siRNA 1569 | 1569 | AGAGATTTACTTTTTTAAA | 3311 | TTTAAAAAAGTAAATCTCT |
| siRNA 1570 | 1570 | GAGATTTACTTTTTTAAAG | 3312 | CTTTAAAAAAGTAAATCTC |
| siRNA 1571 | 1571 | AGATTTACTTTTTTAAAGG | 3313 | CCTTTAAAAAAGTAAATCT |
| siRNA 1572 | 1572 | GATTTACTTTTTTAAAGGA | 3314 | TCCTTTAAAAAAGTAAATC |
| siRNA 1573 | 1573 | ATTTACTTTTTTAAAGGAC | 3315 | GTCCTTTAAAAAAGTAAAT |
| siRNA 1574 | 1574 | TTTACTTTTTTAAAGGACT | 3316 | AGTCCTTTAAAAAAGTAAA |
| siRNA 1575 | 1575 | TTACTTTTTTAAAGGACTT | 3317 | AAGTCCTTTAAAAAAGTAA |
| siRNA 1576 | 1576 | TACTTTTTTAAAGGACTTT | 3318 | AAAGTCCTTTAAAAAAGTA |
| siRNA 1577 | 1577 | ACTTTTTTAAAGGACTTTA | 3319 | TAAAGTCCTTTAAAAAAGT |
| siRNA 1578 | 1578 | CTTTTTTAAAGGACTTTAT | 3320 | ATAAAGTCCTTTAAAAAAG |
| siRNA 1579 | 1579 | TTTTTTAAAGGACTTTATC | 3321 | GATAAAGTCCTTTAAAAAA |
| siRNA 1580 | 1580 | TTTTTAAAGGACTTTATCT | 3322 | AGATAAAGTCCTTTAAAAA |
| siRNA 1581 | 1581 | TTTTAAAGGACTTTATCTG | 3323 | CAGATAAAGTCCTTTAAAA |
| siRNA 1582 | 1582 | TTTAAAGGACTTTATCTGA | 3324 | TCAGATAAAGTCCTTTAAA |
| siRNA 1583 | 1583 | TTAAAGGACTTTATCTGAA | 3325 | TTCAGATAAAGTCCTTTAA |
| siRNA 1584 | 1584 | TAAAGGACTTTATCTGAAC | 3326 | GTTCAGATAAAGTCCTTTA |
| siRNA 1585 | 1585 | AAAGGACTTTATCTGAACA | 3327 | TGTTCAGATAAAGTCCTTT |
| siRNA 1586 | 1586 | AAGGACTTTATCTGAACAG | 3328 | CTGTTCAGATAAAGTCCTT |
| siRNA 1587 | 1587 | AGGACTTTATCTGAACAGA | 3329 | TCTGTTCAGATAAAGTCCT |
| siRNA 1588 | 1588 | GGACTTTATCTGAACAGAG | 3330 | CTCTGTTCAGATAAAGTCC |
| siRNA 1589 | 1589 | GACTTTATCTGAACAGAGA | 3331 | TCTCTGTTCAGATAAAGTC |
| siRNA 1590 | 1590 | ACTTTATCTGAACAGAGAG | 3332 | CTCTCTGTTCAGATAAAGT |
| siRNA 1591 | 1591 | CTTTATCTGAACAGAGAGA | 3333 | TCTCTCTGTTCAGATAAAG |
| siRNA 1592 | 1592 | TTTATCTGAACAGAGAGAT | 3334 | ATCTCTCTGTTCAGATAAA |
| siRNA 1593 | 1593 | TTATCTGAACAGAGAGATA | 3335 | TATCTCTCTGTTCAGATAA |
| siRNA 1594 | 1594 | TATCTGAACAGAGAGATAT | 3336 | ATATCTCTCTGTTCAGATA |
| siRNA 1595 | 1595 | ATCTGAACAGAGAGATATA | 3337 | TATATCTCTCTGTTCAGAT |
| siRNA 1596 | 1596 | TCTGAACAGAGAGATATAA | 3338 | TTATATCTCTCTGTTCAGA |
| siRNA 1597 | 1597 | CTGAACAGAGAGATATAAT | 3339 | ATTATATCTCTCTGTTCAG |
| siRNA 1598 | 1598 | TGAACAGAGAGATATAATA | 3340 | TATTATATCTCTCTGTTCA |
| siRNA 1599 | 1599 | GAACAGAGAGATATAATAT | 3341 | ATATTATATCTCTCTGTTC |
| siRNA 1600 | 1600 | AACAGAGAGATATAATATT | 3342 | AATATTATATCTCTCTGTT |
| siRNA 1601 | 1601 | ACAGAGAGATATAATATTT | 3343 | AAATATTATATCTCTCTGT |
| siRNA 1602 | 1602 | CAGAGAGATATAATATTTT | 3344 | AAAATATTATATCTCTCTG |
| siRNA 1603 | 1603 | AGAGAGATATAATATTTTT | 3345 | AAAAATATTATATCTCTCT |
| siRNA 1604 | 1604 | GAGAGATATAATATTTTTC | 3346 | GAAAAATATTATATCTCTC |
| siRNA 1605 | 1605 | AGAGATATAATATTTTTCC | 3347 | GGAAAAATATTATATCTCT |
| siRNA 1606 | 1606 | GAGATATAATATTTTTCCT | 3348 | AGGAAAAATATTATATCTC |
| siRNA 1607 | 1607 | AGATATAATATTTTTCCTA | 3349 | TAGGAAAAATATTATATCT |
| siRNA 1608 | 1608 | GATATAATATTTTTCCTAT | 3350 | ATAGGAAAAATATTATATC |
| siRNA 1609 | 1609 | ATATAATATTTTTCCTATT | 3351 | AATAGGAAAAATATTATAT |
| siRNA 1610 | 1610 | TATAATATTTTTCCTATTG | 3352 | CAATAGGAAAAATATTATA |
| siRNA 1611 | 1611 | ATAATATTTTTCCTATTGG | 3353 | CCAATAGGAAAAATATTAT |
| siRNA 1612 | 1612 | TAATATTTTTCCTATTGGA | 3354 | TCCAATAGGAAAAATATTA |
| siRNA 1613 | 1613 | AATATTTTTCCTATTGGAC | 3355 | GTCCAATAGGAAAAATATT |
| siRNA 1614 | 1614 | ATATTTTTCCTATTGGACA | 3356 | TGTCCAATAGGAAAAATAT |
| siRNA 1615 | 1615 | TATTTTTCCTATTGGACAA | 3357 | TTGTCCAATAGGAAAAATA |
| siRNA 1616 | 1616 | ATTTTTCCTATTGGACAAT | 3358 | ATTGTCCAATAGGAAAAAT |
| siRNA 1617 | 1617 | TTTTTCCTATTGGACAATG | 3359 | CATTGTCCAATAGGAAAAA |
| siRNA 1618 | 1618 | TTTTCCTATTGGACAATGG | 3360 | CCATTGTCCAATAGGAAAA |
| siRNA 1619 | 1619 | TTTCCTATTGGACAATGGA | 3361 | TCCATTGTCCAATAGGAAA |
| siRNA 1620 | 1620 | TTCCTATTGGACAATGGAC | 3362 | GTCCATTGTCCAATAGGAA |
| siRNA 1621 | 1621 | TCCTATTGGACAATGGACT | 3363 | AGTCCATTGTCCAATAGGA |
| siRNA 1622 | 1622 | CCTATTGGACAATGGACTT | 3364 | AAGTCCATTGTCCAATAGG |
| siRNA 1623 | 1623 | CTATTGGACAATGGACTTG | 3365 | CAAGTCCATTGTCCAATAG |
| siRNA 1624 | 1624 | TATTGGACAATGGACTTGC | 3366 | GCAAGTCCATTGTCCAATA |
| siRNA 1625 | 1625 | ATTGGACAATGGACTTGCA | 3367 | TGCAAGTCCATTGTCCAAT |
| siRNA 1626 | 1626 | TTGGACAATGGACTTGCAA | 3368 | TTGCAAGTCCATTGTCCAA |
| siRNA 1627 | 1627 | TGGACAATGGACTTGCAAA | 3369 | TTTGCAAGTCCATTGTCCA |
| siRNA 1628 | 1628 | GGACAATGGACTTGCAAAG | 3370 | CTTTGCAAGTCCATTGTCC |
| siRNA 1629 | 1629 | GACAATGGACTTGCAAAGC | 3371 | GCTTTGCAAGTCCATTGTC |
| siRNA 1630 | 1630 | ACAATGGACTTGCAAAGCT | 3372 | AGCTTTGCAAGTCCATTGT |
| siRNA 1631 | 1631 | CAATGGACTTGCAAAGCTT | 3373 | AAGCTTTGCAAGTCCATTG |
| siRNA 1632 | 1632 | AATGGACTTGCAAAGCTTC | 3374 | GAAGCTTTGCAAGTCCATT |
| siRNA 1633 | 1633 | ATGGACTTGCAAAGCTTCA | 3375 | TGAAGCTTTGCAAGTCCAT |
| siRNA 1634 | 1634 | TGGACTTGCAAAGCTTCAC | 3376 | GTGAAGCTTTGCAAGTCCA |
| siRNA 1635 | 1635 | GGACTTGCAAAGCTTCACT | 3377 | AGTGAAGCTTTGCAAGTCC |
| siRNA 1636 | 1636 | GACTTGCAAAGCTTCACTT | 3378 | AAGTGAAGCTTTGCAAGTC |
| siRNA 1637 | 1637 | ACTTGCAAAGCTTCACTTC | 3379 | GAAGTGAAGCTTTGCAAGT |
| siRNA 1638 | 1638 | CTTGCAAAGCTTCACTTCA | 3380 | TGAAGTGAAGCTTTGCAAG |
| siRNA 1639 | 1639 | TTGCAAAGCTTCACTTCAT | 3381 | ATGAAGTGAAGCTTTGCAA |
| siRNA 1640 | 1640 | TGCAAAGCTTCACTTCATT | 3382 | AATGAAGTGAAGCTTTGCA |
| siRNA 1641 | 1641 | GCAAAGCTTCACTTCATTT | 3383 | AAATGAAGTGAAGCTTTGC |
| siRNA 1642 | 1642 | CAAAGCTTCACTTCATTTT | 3384 | AAAATGAAGTGAAGCTTTG |
| siRNA 1643 | 1643 | AAAGCTTCACTTCATTTTA | 3385 | TAAAATGAAGTGAAGCTTT |
| siRNA 1644 | 1644 | AAGCTTCACTTCATTTTAA | 3386 | TTAAAATGAAGTGAAGCTT |
| siRNA 1645 | 1645 | AGCTTCACTTCATTTTAAG | 3387 | CTTAAAATGAAGTGAAGCT |
| siRNA 1646 | 1646 | GCTTCACTTCATTTTAAGA | 3388 | TCTTAAAATGAAGTGAAGC |
| siRNA 1647 | 1647 | CTTCACTTCATTTTAAGAG | 3389 | CTCTTAAAATGAAGTGAAG |
| siRNA 1648 | 1648 | TTCACTTCATTTTAAGAGC | 3390 | GCTCTTAAAATGAAGTGAA |
| siRNA 1649 | 1649 | TCACTTCATTTTAAGAGCA | 3391 | TGCTCTTAAAATGAAGTGA |
| siRNA 1650 | 1650 | CACTTCATTTTAAGAGCAA | 3392 | TTGCTCTTAAAATGAAGTG |
| siRNA 1651 | 1651 | ACTTCATTTTAAGAGCAAA | 3393 | TTTGCTCTTAAAATGAAGT |
| siRNA 1652 | 1652 | CTTCATTTTAAGAGCAAAA | 3394 | TTTTGCTCTTAAAATGAAG |
| siRNA 1653 | 1653 | TTCATTTTAAGAGCAAAAG | 3395 | CTTTTGCTCTTAAAATGAA |
| siRNA 1654 | 1654 | TCATTTTAAGAGCAAAAGA | 3396 | TCTTTTGCTCTTAAAATGA |
| siRNA 1655 | 1655 | CATTTTAAGAGCAAAAGAC | 3397 | GTCTTTTGCTCTTAAAATG |
| siRNA 1656 | 1656 | ATTTTAAGAGCAAAAGACC | 3398 | GGTCTTTTGCTCTTAAAAT |
| siRNA 1657 | 1657 | TTTTAAGAGCAAAAGACCC | 3399 | GGGTCTTTTGCTCTTAAAA |
| siRNA 1658 | 1658 | TTTAAGAGCAAAAGACCCC | 3400 | GGGGTCTTTTGCTCTTAAA |
| siRNA 1659 | 1659 | TTAAGAGCAAAAGACCCCA | 3401 | TGGGGTCTTTTGCTCTTAA |
| siRNA 1660 | 1660 | TAAGAGCAAAAGACCCCAT | 3402 | ATGGGGTCTTTTGCTCTTA |
| siRNA 1661 | 1661 | AAGAGCAAAAGACCCCATG | 3403 | CATGGGGTCTTTTGCTCTT |
| siRNA 1662 | 1662 | AGAGCAAAAGACCCCATGT | 3404 | ACATGGGGTCTTTTGCTCT |
| siRNA 1663 | 1663 | GAGCAAAAGACCCCATGTT | 3405 | AACATGGGGTCTTTTGCTC |
| siRNA 1664 | 1664 | AGCAAAAGACCCCATGTTG | 3406 | CAACATGGGGTCTTTTGCT |
| siRNA 1665 | 1665 | GCAAAAGACCCCATGTTGA | 3407 | TCAACATGGGGTCTTTTGC |
| siRNA 1666 | 1666 | CAAAAGACCCCATGTTGAA | 3408 | TTCAACATGGGGTCTTTTG |
| siRNA 1667 | 1667 | AAAAGACCCCATGTTGAAA | 3409 | TTTCAACATGGGGTCTTTT |
| siRNA 1668 | 1668 | AAAGACCCCATGTTGAAAA | 3410 | TTTTCAACATGGGGTCTTT |
| siRNA 1669 | 1669 | AAGACCCCATGTTGAAAAC | 3411 | GTTTTCAACATGGGGTCTT |
| siRNA 1670 | 1670 | AGACCCCATGTTGAAAACT | 3412 | AGTTTTCAACATGGGGTCT |
| siRNA 1671 | 1671 | GACCCCATGTTGAAAACTC | 3413 | GAGTTTTCAACATGGGGTC |
| siRNA 1672 | 1672 | ACCCCATGTTGAAAACTCC | 3414 | GGAGTTTTCAACATGGGGT |
| siRNA 1673 | 1673 | CCCCATGTTGAAAACTCCA | 3415 | TGGAGTTTTCAACATGGGG |
| siRNA 1674 | 1674 | CCCATGTTGAAAACTCCAT | 3416 | ATGGAGTTTTCAACATGGG |
| siRNA 1675 | 1675 | CCATGTTGAAAACTCCATA | 3417 | TATGGAGTTTTCAACATGG |
| siRNA 1676 | 1676 | CATGTTGAAAACTCCATAA | 3418 | TTATGGAGTTTTCAACATG |
| siRNA 1677 | 1677 | ATGTTGAAAACTCCATAAC | 3419 | GTTATGGAGTTTTCAACAT |
| siRNA 1678 | 1678 | TGTTGAAAACTCCATAACA | 3420 | TGTTATGGAGTTTTCAACA |
| siRNA 1679 | 1679 | GTTGAAAACTCCATAACAG | 3421 | CTGTTATGGAGTTTTCAAC |
| siRNA 1680 | 1680 | TTGAAAACTCCATAACAGT | 3422 | ACTGTTATGGAGTTTTCAA |
| siRNA 1681 | 1681 | TGAAAACTCCATAACAGTT | 3423 | AACTGTTATGGAGTTTTCA |
| siRNA 1682 | 1682 | GAAAACTCCATAACAGTTT | 3424 | AAACTGTTATGGAGTTTTC |
| siRNA 1683 | 1683 | AAAACTCCATAACAGTTTT | 3425 | AAAACTGTTATGGAGTTTT |
| siRNA 1684 | 1684 | AAACTCCATAACAGTTTTA | 3426 | TAAAACTGTTATGGAGTTT |
| siRNA 1685 | 1685 | AACTCCATAACAGTTTTAT | 3427 | ATAAAACTGTTATGGAGTT |
| siRNA 1686 | 1686 | ACTCCATAACAGTTTTATG | 3428 | CATAAAACTGTTATGGAGT |
| siRNA 1687 | 1687 | CTCCATAACAGTTTTATGC | 3429 | GCATAAAACTGTTATGGAG |
| siRNA 1688 | 1688 | TCCATAACAGTTTTATGCT | 3430 | AGCATAAAACTGTTATGGA |
| siRNA 1689 | 1689 | CCATAACAGTTTTATGCTG | 3431 | CAGCATAAAACTGTTATGG |
| siRNA 1690 | 1690 | CATAACAGTTTTATGCTGA | 3432 | TCAGCATAAAACTGTTATG |
| siRNA 1691 | 1691 | ATAACAGTTTTATGCTGAT | 3433 | ATCAGCATAAAACTGTTAT |
| siRNA 1692 | 1692 | TAACAGTTTTATGCTGATG | 3434 | CATCAGCATAAAACTGTTA |
| siRNA 1693 | 1693 | AACAGTTTTATGCTGATGA | 3435 | TCATCAGCATAAAACTGTT |
| siRNA 1694 | 1694 | ACAGTTTTATGCTGATGAT | 3436 | ATCATCAGCATAAAACTGT |
| siRNA 1695 | 1695 | CAGTTTTATGCTGATGATA | 3437 | TATCATCAGCATAAAACTG |
| siRNA 1696 | 1696 | AGTTTTATGCTGATGATAA | 3438 | TTATCATCAGCATAAAACT |
| siRNA 1697 | 1697 | GTTTTATGCTGATGATAAT | 3439 | ATTATCATCAGCATAAAAC |
| siRNA 1698 | 1698 | TTTTATGCTGATGATAATT | 3440 | AATTATCATCAGCATAAAA |
| siRNA 1699 | 1699 | TTTATGCTGATGATAATTT | 3441 | AAATTATCATCAGCATAAA |
| siRNA 1700 | 1700 | TTATGCTGATGATAATTTA | 3442 | TAAATTATCATCAGCATAA |
| siRNA 1701 | 1701 | TATGCTGATGATAATTTAT | 3443 | ATAAATTATCATCAGCATA |
| siRNA 1702 | 1702 | ATGCTGATGATAATTTATC | 3444 | GATAAATTATCATCAGCAT |
| siRNA 1703 | 1703 | TGCTGATGATAATTTATCT | 3445 | AGATAAATTATCATCAGCA |
| siRNA 1704 | 1704 | GCTGATGATAATTTATCTA | 3446 | TAGATAAATTATCATCAGC |
| siRNA 1705 | 1705 | CTGATGATAATTTATCTAC | 3447 | GTAGATAAATTATCATCAG |
| siRNA 1706 | 1706 | TGATGATAATTTATCTACA | 3448 | TGTAGATAAATTATCATCA |
| siRNA 1707 | 1707 | GATGATAATTTATCTACAT | 3449 | ATGTAGATAAATTATCATC |
| siRNA 1708 | 1708 | ATGATAATTTATCTACATG | 3450 | CATGTAGATAAATTATCAT |
| siRNA 1709 | 1709 | TGATAATTTATCTACATGC | 3451 | GCATGTAGATAAATTATCA |
| siRNA 1710 | 1710 | GATAATTTATCTACATGCA | 3452 | TGCATGTAGATAAATTATC |
| siRNA 1711 | 1711 | ATAATTTATCTACATGCAT | 3453 | ATGCATGTAGATAAATTAT |
| siRNA 1712 | 1712 | TAATTTATCTACATGCATT | 3454 | AATGCATGTAGATAAATTA |
| siRNA 1713 | 1713 | AATTTATCTACATGCATTT | 3455 | AAATGCATGTAGATAAATT |
| siRNA 1714 | 1714 | ATTTATCTACATGCATTTC | 3456 | GAAATGCATGTAGATAAAT |
| siRNA 1715 | 1715 | TTTATCTACATGCATTTCA | 3457 | TGAAATGCATGTAGATAAA |
| siRNA 1716 | 1716 | TTATCTACATGCATTTCAA | 3458 | TTGAAATGCATGTAGATAA |
| siRNA 1717 | 1717 | TATCTACATGCATTTCAAT | 3459 | ATTGAAATGCATGTAGATA |
| siRNA 1718 | 1718 | ATCTACATGCATTTCAATA | 3460 | TATTGAAATGCATGTAGAT |
| siRNA 1719 | 1719 | TCTACATGCATTTCAATAA | 3461 | TTATTGAAATGCATGTAGA |
| siRNA 1720 | 1720 | CTACATGCATTTCAATAAA | 3462 | TTTATTGAAATGCATGTAG |
| siRNA 1721 | 1721 | TACATGCATTTCAATAAAC | 3463 | GTTTATTGAAATGCATGTA |
| siRNA 1722 | 1722 | ACATGCATTTCAATAAACC | 3464 | GGTTTATTGAAATGCATGT |
| siRNA 1723 | 1723 | CATGCATTTCAATAAACCT | 3465 | AGGTTTATTGAAATGCATG |
| siRNA 1724 | 1724 | ATGCATTTCAATAAACCTT | 3466 | AAGGTTTATTGAAATGCAT |
| siRNA 1725 | 1725 | TGCATTTCAATAAACCTTT | 3467 | AAAGGTTTATTGAAATGCA |
| siRNA 1726 | 1726 | GCATTTCAATAAACCTTTT | 3468 | AAAAGGTTTATTGAAATGC |
| siRNA 1727 | 1727 | CATTTCAATAAACCTTTTG | 3469 | CAAAAGGTTTATTGAAATG |
| siRNA 1728 | 1728 | ATTTCAATAAACCTTTTGT | 3470 | ACAAAAGGTTTATTGAAAT |
| siRNA 1729 | 1729 | TTTCAATAAACCTTTTGTT | 3471 | AACAAAAGGTTTATTGAAA |
| siRNA 1730 | 1730 | TTCAATAAACCTTTTGTTT | 3472 | AAACAAAAGGTTTATTGAA |
| siRNA 1731 | 1731 | TCAATAAACCTTTTGTTTC | 3473 | GAAACAAAAGGTTTATTGA |
| siRNA 1732 | 1732 | CAATAAACCTTTTGTTTCC | 3474 | GGAAACAAAAGGTTTATTG |
| siRNA 1733 | 1733 | AATAAACCTTTTGTTTCCT | 3475 | AGGAAACAAAAGGTTTATT |
| siRNA 1734 | 1734 | ATAAACCTTTTGTTTCCTA | 3476 | TAGGAAACAAAAGGTTTAT |
| siRNA 1735 | 1735 | TAAACCTTTTGTTTCCTAA | 3477 | TTAGGAAACAAAAGGTTTA |
| siRNA 1736 | 1736 | AAACCTTTTGTTTCCTAAG | 3478 | CTTAGGAAACAAAAGGTTT |
| siRNA 1737 | 1737 | AACCTTTTGTTTCCTAAGA | 3479 | TCTTAGGAAACAAAAGGTT |
| siRNA 1738 | 1738 | ACCTTTTGTTTCCTAAGAC | 3480 | GTCTTAGGAAACAAAAGGT |
| siRNA 1739 | 1739 | CCTTTTGTTTCCTAAGACT | 3481 | AGTCTTAGGAAACAAAAGG |
| siRNA 1740 | 1740 | CTTTTGTTTCCTAAGACTA | 3482 | TAGTCTTAGGAAACAAAAG |
| siRNA 1741 | 1741 | TTTTGTTTCCTAAGACTAG | 3483 | CTAGTCTTAGGAAACAAAA |
| siRNA 1742 | 1742 | TTTGTTTCCTAAGACTAGA | 3484 | TCTAGTCTTAGGAAACAAA |

TABLE 80
Additional Sequences

| SEQ ID | |
|---|---|
| NO: | 5′ to 3′ Sequence |
| 3621 | AAAAAGGAGGAGCTTCAACCTGTGTGCAAAATCTGGGAACCTGACAGTATAGGTTG |
| | GGGGCCAGGATGAGGAAAAAGGAACGGGAAAGACCTGCCCACCCTTCTGGTAAGG |
| | AGGCCCCGTGATCAGCTCCAGCCATTTGCAGTCCTGGCTATCCCAGGAGCTTACATA |
| | AAGGGACAATTGGAGCCTGAGAGGTGACAGTGCTGACACTACAAGGCTCGGAGCT |
| | CCGGGCACTCAGACATCATGAGTTGGTCCTTGCACCCCCGGAATTTAATTCTCTACT |
| | TCTATGCTCTTTTATTTCTCTCTTCAACATGTGTAGCATATGTTGCTACCAGAGACAA |
| | CTGCTGCATCTTAGATGAAAGATTCGGTAGTTATTGTCCAACTACCTGTGGCATTGC |
| | AGATTTCCTGTCTACTTATCAAACCAAAGTAGACAAGGATCTACAGTCTTTGGAAG |
| | ACATCTTACATCAAGTTGAAAACAAAACATCAGAAGTCAAACAGCTGATAAAAGC |
| | AATCCAACTCACTTATAATCCTGATGAATCATCAAAACCAAATATGATAGACGCTG |
| | CTACTTTGAAGTCCAGGAAAATGTTAGAAGAAATTATGAAATATGAAGCATCGATT |
| | TTAACACATGACTCAAGTATTCGATATTTGCAGGAAATATATAATTCAAATAATCA |
| | AAAGATTGTTAACCTGAAAGAGAAGGTAGCCCAGCTTGAAGCACAGTGCCAGGAA |
| | CCTTGCAAAGACACGGTGCAAATCCATGATATCACTGGGAAAGATTGTCAAGACAT |
| | TGCCAATAAGGGAGCTAAACAGAGCGGGCTTTACTTTATTAAACCTCTGAAAGCTA |
| | ACCAGCAATTCTTAGTCTACTGTGAAATCGATGGGTCTGGAAATGGATGGACTGTG |
| | TTTCAGAAGAGACTTGATGGCAGTGTAGATTTCAAGAAAAACTGGATTCAATATAA |
| | AGAAGGATTTGGACATCTGTCTCCTACTGGCACAACAGAATTTTGGCTGGGAAATG |
| | AGAAGATTCATTTGATAAGCACACAGTCTGCCATCCCATATGCATTAAGAGTGGAA |
| | CTGGAAGACTGGAATGGCAGAACCAGTACTGCAGACTATGCCATGTTCAAGGTGGG |
| | ACCTGAAGCTGACAAGTACCGCCTAACATATGCCTACTTCGCTGGTGGGGATGCTG |
| | GAGATGCCTTTGATGGCTTTGATTTTGGCGATGATCCTAGTGACAAGTTTTTCACAT |
| | CCCATAATGGCATGCAGTTCAGTACCTGGGACAATGACAATGATAAGTTTGAAGGC |
| | AACTGTGCTGAACAGGATGGATCTGGTTGGTGGATGAACAAGTGTCACGCTGGCCA |
| | TCTCAATGGAGTTTATTACCAAGGTGGCACTTACTCAAAAGCATCTACTCCTAATGG |
| | TTATGATAATGGCATTATTTGGGCCACTTGGAAAACCCGGTGGTATTCCATGAAGA |
| | AAACCACTATGAAGATAATCCCATTCAACAGACTCACAATTGGAGAAGGACAGCA |
| | ACACCACCTGGGGGGAGCCAAACAGGCTGGAGACGTTTAAAAGACCGTTTCAAAA |
| | GAGATTTACTTTTTTAAAGGACTTTATCTGAACAGAGAGATATAATATTTTTCCTAT |
| | TGGACAATGGACTTGCAAAGCTTCACTTCATTTTAAGAGCAAAAGACCCCATGTTG |
| | AAAACTCCATAACAGTTTTATGCTGATGATAATTTATCTACATGCATTTCAATAAAC |
| | CTTTTGTTTCCTAAGACTAGA |
| 3622 | NfsnsNfnNfnNfNfNfnNfnNfnNfnNfnNfsnsn |
| 3623 | nsnsnnNfnNfNfNfnnnnnnnnnnsnsn |
| 3624 | nsnsnnNfnNfnNfnnnnnnnnnnsnsn |
| 3625 | NfsnsNfnNfnNfNfNfnNfnNfnNfnNfnNfsnsnN-moiety (e.g. a sugar moiety) |
| 3626 | nsnsnnNfnNfNfNfnnnnnnnnnnsnsnN-moiety (e.g. sugar moiety) |
| 3627 | NfsnsNfnNfnNfnNfnNfnNfnNfnNfnNfsnsn |
| 3628 | nsnsnnNfNfNfNfNfnnnnnnnnnnsnsn |
| 3629 | nsnsnnnNfNfNfNfnnnnnnnnnnsnsn |
| 3630 | nsnsnnnnNfNfNfNfnnnnnnnnnsnsn |
| 3631 | nsNfsnNfnNfnNfnNfnnnNfnNfnNfnsnsn |
| 3632 | nsNfsnnnNfnNfNfnnnnNfnNfnnnsnsn |
| 3633 | nsNfsnnnNfnnnnnnnNfnNfnnnsnsn |
| 3634 | nsNfsnNfnNfnnnnnnnNfnNfnnnsnsn |
| 3635 | nsNfsnnnnnnnnnnnNfnNfnnnsnsn |
| 3636 | nsNfsnnnNfnnNfnnnnNfnNfnnnsnsn |
| 3637 | nsNfsnNfnNfnNfnNfnNfnNfnNfnNfnsnsn |
| 3638 | nsNfsnnnnnnnnnnnNfnnnnnsnsn |
| 3639 | nNfnNfnNfnNfnNfnNfnNfnNfnNfnsnsn |
| 3640 | nsnsnsnsnsdNsdNsdNsdNsdNsdNsdNsdNsdNsdNsnsnsnsnsn |

TABLE 81
Modified siRNA Sequences

| | SEQ | | SEQ | |
|---|---|---|---|---|
| siRNA | ID | | ID | |
| Name | NO: | Sense strand sequence (5′-3′) | NO: | Antisense strand sequence (5′-3′) |
| siRNA 3599 | 3599 | [ETL1]gsasugaAfaGfAfuucgguaguasusu | 3595 | usAfscUfaCfcGfaAfuCfuUfuCfaUfcsusu |
| siRNA 3600 | 3600 | [ETL1]gsasugaAfAfgAfuucgguaguasusu | 3595 | usAfscUfaCfcGfaAfuCfuUfuCfaUfcsusu |
| siRNA 3601 | 3601 | [ETL1]gsasugaAfAfGfAfuucgguaguasusu | 3595 | usAfscUfaCfcGfaAfuCfuUfuCfaUfcsusu |
| siRNA 3602 | 3602 | [ETL1]gsasugAfaaGfAfuucgguaguasusu | 3595 | usAfscUfaCfcGfaAfuCfuUfuCfaUfcsusu |
| siRNA 3603 | 3603 | [ETL1]gsasugAfaAfgAfuucgguaguasusu | 3595 | usAfscUfaCfcGfaAfuCfuUfuCfaUfcsusu |
| siRNA 3604 | 3604 | [ETL1]gsasugAfAfagAfuucgguaguasusu | 3595 | usAfscUfaCfcGfaAfuCfuUfuCfaUfcsusu |
| ETD01592 | 3591 | [ETL1]gsasugAfAfaGfAfuucgguaguasusu | 3595 | usAfscUfaCfcGfaAfuCfuUfuCfaUfcsusu |
| siRNA 3605 | 3605 | [ETL1]gsasugAfAfAfGfAfuucgguaguasusu | 3595 | usAfscUfaCfcGfaAfuCfuUfuCfaUfcsusu |
| siRNA 3606 | 3606 | [ETL1]gsgsaaaugAfGfaAfgauucauasusu | 3596 | usAfsuGfaAfuCfuUfcUfcAfuUfuCfcsusu |
| siRNA 3607 | 3607 | [ETL1]gsgsaaaugAfGfAfAfgauucauasusu | 3596 | usAfsuGfaAfuCfuUfcUfcAfuUfuCfcsusu |
| siRNA 3608 | 3608 | [ETL1]gsgsaaauGfaGfaAfgauucauasusu | 3596 | usAfsuGfaAfuCfuUfcUfcAfuUfuCfcsusu |
| ETD01593 | 3592 | [ETL1]gsgsaaauGfaGfAfagauucauasusu | 3596 | usAfsuGfaAfuCfuUfcUfcAfuUfuCfcsusu |
| siRNA 3609 | 3609 | [ETL1]gsgsaaauGfAfGfAfagauucauasusu | 3596 | usAfsuGfaAfuCfuUfcUfcAfuUfuCfcsusu |
| siRNA 3610 | 3610 | [ETL1]gsgsaaauGfAfGfAfAfgauucauasusu | 3596 | usAfsuGfaAfuCfuUfcUfcAfuUfuCfcsusu |
| siRNA 3611 | 3611 | [ETL1]gsgsaaAfugaGfaAfgauucauasusu | 3596 | usAfsuGfaAfuCfuUfcUfcAfuUfuCfcsusu |
| siRNA 3612 | 3612 | [ETL1]gsgsaaAfugaGfAfagauucauasusu | 3596 | usAfsuGfaAfuCfuUfcUfcAfuUfuCfcsusu |
| siRNA 3613 | 3613 | [ETL1]gsgsaaAfugAfGfaagauucauasusu | 3596 | usAfsuGfaAfuCfuUfcUfcAfuUfuCfcsusu |
| siRNA 3614 | 3614 | [ETL1]gsgsaaAfugAfGfaAfgauucauasusu | 3596 | usAfsuGfaAfuCfuUfcUfcAfuUfuCfcsusu |
| siRNA 3615 | 3615 | [ETL1]gsgsaaAfugAfGfAfAfgauucauasusu | 3596 | usAfsuGfaAfuCfuUfcUfcAfuUfuCfcsusu |
| siRNA 3616 | 3616 | [ETL1]gsgsaaAfuGfaGfaagauucauasusu | 3596 | usAfsuGfaAfuCfuUfcUfcAfuUfuCfcsusu |
| siRNA 3617 | 3617 | [ETL1]gsgsaaAfuGfaGfaAfgauucauasusu | 3596 | usAfsuGfaAfuCfuUfcUfcAfuUfuCfcsusu |
| siRNA 3618 | 3618 | [ETL1]gsgsaaAfuGfaGfAfagauucauasusu | 3596 | usAfsuGfaAfuCfuUfcUfcAfuUfuCfcsusu |
| siRNA 3619 | 3619 | [ETL1]gsgsaaAfuGfAfGfAfagauucauasusu | 3596 | usAfsuGfaAfuCfuUfcUfcAfuUfuCfcsusu |
| ETD01594 | 3593 | [ETL1]gsasagAfuucAfuuugauaagasusu | 3597 | usCfsuUfaUfcAfaAfuGfaAfuCfuUfcsusu |
| SIRNA 3620 | 3620 | [ETL1]usasaguuuGfAfaGfgcaacugasusu | 3598 | usCfsaGfuUfgCfcUfuCfaAfaCfuUfasusu |
| ETD01595 | 3594 | [ETL1]usasaguuuGfAfAfGfgcaacugasusu | 3598 | usCfsaGfuUfgCfcUfuCfaAfaCfuUfasusu |

[ETL1] = GalNAc#1 (shown connected 5′ to the sense strand)

What is claimed is:

1. A composition comprising an oligonucleotide that targets FGG and when administered to a subject in an effective amount improves a mental disorder measurement in the subject, relative to a baseline mental disorder measurement.

2. The composition of claim 1, wherein the mental disorder comprises a psychiatric disorder.

3. The composition of claim 2, wherein the psychiatric disorder comprises a depressive disorder, post-traumatic stress disorder, mood disorder, anxiety disorder, eating disorder, substance-use disorder, bipolar disorder, personality disorder, schizophrenia, or schizoaffective disorder.

4. The composition of claim 1, wherein the mental disorder measurement comprises a Montgomery-Asberg Depression Rating Scale score, a Hamilton Depression Rating Scale score, or a measurement of an anxiety disorder, depressive disorder, eating disorder, substance-use disorder, post-traumatic stress disorder, bipolar disorder, schizophrenia, or psychosis sign or symptom.

5. The composition of claim 1, wherein the mental disorder comprises a neurological disorder.

6. The composition of claim 5, wherein the neurological disorder comprises Alzheimer's disease, dementia, delirium, cognitive decline, vascular dementia, headache, chronic pain, chronic fatigue syndrome, or motor neuron disease.

7. The composition of claim 6, wherein the mental disorder measurement comprises a measurement of cognitive function, CNS amyloid plaques, CNS tau accumulation, CSF beta-amyloid 42, CSF tau, CSF phospho-tau, Lewy bodies, CSF alpha-synuclein, or a sign or symptom of headache, migraine, chronic pain, fibromyalgia, chronic fatigue syndrome, or motor neuron disease.

8. A composition comprising an oligonucleotide that targets FGG and when administered to a subject in an effective amount decreases fibrinogen.

9. The composition of claim 8, wherein the composition decreases circulating fibrinogen.

10. The composition of claim 8, wherein the subject has a clotting or coagulation disorder.

11. The composition of claim 8, wherein the subject has a thrombophilia.

12. The composition of claim 8, wherein the subject has a venous thromboembolism.

13. The composition of any one of the preceding claims, wherein the oligonucleotide comprises a modified internucleoside linkage.

14. The composition of claim 13, wherein the modified internucleoside linkage comprises alkylphosphonate, phosphorothioate, methylphosphonate, phosphorodithioate, alkylphosphonothioate, phosphoramidate, carbamate, carbonate, phosphate triester, acetamidate, or carboxymethyl ester, or a combination thereof.

15. The composition of claim 13, wherein the modified internucleoside linkage comprises one or more phosphorothioate linkages.

16. The composition of any one of the preceding claims, wherein the oligonucleotide comprises 1, 2, 3, 4, 5, 6, 7, 8, 9, 10, 11, 12, 13, 14, 15, 16, 17, 18, 19, or 20 modified internucleoside linkages.

17. The composition of any one of the preceding claims, wherein the oligonucleotide comprises a modified nucleoside.

18. The composition of claim 17, wherein the modified nucleoside comprises a locked nucleic acid (LNA), hexitol nucleic acid (HLA), cyclohexene nucleic acid (CeNA)′ 2′-methoxyethyl′ 2′-O-alkyl′ 2′-O-allyl′ 2′-O-allyl′ 2′-fluoro, o′ 2′-deoxy, or a combination thereof.

19. The composition of claim 17, wherein the modified nucleoside comprises a LNA.

20. The composition of claim 17, wherein the modified nucleoside comprises a 2′,4′ constrained ethyl nucleic acid.

21. The composition of claim 17, wherein the modified nucleoside comprises ′2′-O-methyl nucleoside′ 2′-deoxyfluoro nucleoside′ 2′-O—N-methylacetamido′(2′-O-NMA) nucleoside, ′ 2′-O-dimethylaminoethoxyethyl′(2′-O-DMAEOE) nucleoside′ 2′-O-aminopropyl′(2′-O-AP) nucleoside, o′ 2′-ara-F, or a combination thereof.

22. The composition of claim 17, wherein the modified nucleoside comprises one or more 2′fluoro modified nucleosides.

23. The composition of claim 17, wherein the modified nucleoside comprises ′ 2′ O-alkyl modified nucleoside.

24. The composition of any one of the preceding claims, wherein the oligonucleotide comprises 1, 2, 3, 4, 5, 6, 7, 8, 9, 10, 11, 12, 13, 14, 15, 16, 17, 18, 19, 20, or 21 modified nucleosides.

25. The composition of any one of the preceding claims, wherein the oligonucleotide comprises a sugar moiety attached at a 3′ or 5′ terminus of the oligonucleotide.

26. The composition of claim 25, wherein the sugar comprises N-acetylgalactosamine (GalNAc), N-acetylglucosamine (GlcNAc), or mannose.

27. The composition of claim 25, wherein the sugar comprises GalNAc.

28. The composition of claim 27, wherein the sugar moiety comprises ETL17.

29. The composition of any one of the preceding claims, wherein the oligonucleotide comprises a small interfering RNA (siRNA) comprising a sense strand and an antisense strand.

30. The composition of claim 29, wherein the sense strand is 12-30 nucleosides in length.

31. The composition of claim 29, wherein the antisense strand is 12-30 nucleosides in length.

32. A composition comprising an oligonucleotide that inhibits the expression of FGG, wherein the oligonucleotide comprises an siRNA comprising a sense strand and an antisense strand, each strand is independently about 12-30 nucleosides in length, and at least one of the sense strand and the antisense strand comprises a nucleoside sequence comprising about 12-30 contiguous nucleosides of SEQ ID NO: 3621.

33. The composition of claim 29, wherein any one of the following is true with regard to the sense strand:
all purines comprise 2′ fluoro modified purines, and all pyrimidines comprise a mixture of 2′ fluoro and 2′-O-methyl modified pyrimidines;
all purines comprise 2′-O-methyl modified purines, and all pyrimidines comprise a mixture of 2′ fluoro and 2′-O-methyl modified pyrimidines;
all purines comprise 2′ fluoro modified purines, and all pyrimidines comprise 2′-O-methyl modified pyrimidines;
all pyrimidines comprise 2′ fluoro modified pyrimidines, and all purines comprise a mixture of 2′ fluoro and 2′-O-methyl modified purines;
all pyrimidines comprise 2′-O-methyl modified pyrimidines, and all purines comprise a mixture of 2′ fluoro and 2′-O-methyl modified purines; or
all pyrimidines comprise 2′ fluoro modified pyrimidines, and all purines comprise 2′-O-methyl modified purines.

34. The composition of claim 29, wherein the antisense strand comprises a mixture of 2′ fluoro and 2′-O-methyl modified nucleosides.

35. The composition of claim 1 or 8, wherein the oligonucleotide comprises an antisense oligonucleotide (ASO).

36. The composition of claim 30, wherein the ASO is 12-30 nucleosides in length.

37. A composition comprising an oligonucleotide that inhibits the expression of FGG, wherein the oligonucleotide comprises an ASO about 12-30 nucleosides in length and a nucleoside sequence complementary to about 12-30 contiguous nucleosides of SEQ ID NO: 3621.

38. The composition of any one of claims 1-37, further comprising a pharmaceutically acceptable carrier.

39. A method of treating a subject having a psychiatric disorder or a neurological disorder, comprising administering an effective amount of the composition of claim 38 to the subject.

40. The method of claim 39, wherein the psychiatric disorder comprises a depressive disorder, persistent depressive disorder, treatment resistant depression, a sign or symptom of depression, post-traumatic stress disorder, mood disorder, anxiety disorders, eating disorder, substance-use disorder, bipolar disorder, personality disorder, schizophrenia, or a schizoaffective disorder.

41. The method of claim 39, wherein the neurological disorder comprises Alzheimer's disease, dementia, delirium, cognitive decline, vascular dementia, headache, chronic pain, chronic fatigue syndrome, and motor neuron disease.

42. A method of treating a subject having a clotting or coagulation disorder or a thrombophilia, comprising administering an effective amount of the composition of claim 38 to the subject.

43. The method of claim 42, wherein the prothrombin time, International Normalized Ratio, or the activated partial thromboplastin time is increased compared to a baseline.
